(19) United States
(12) Patent Application Publication
(10) Pub. No.: US 2026/0306614 A1
(43) Pub. Date: Oct. 8, 2026

(54) VACCINE COMPOSITIONS

(71) Applicant: SAIL BIOMEDICINES, INC., Cambridge, MA (US)

(72) Inventors: Emad ARAFA, Cambridge, MA (US); Munir MOSAHEB, Cambridge, MA (US); Siddharth PATEL, Cambridge, MA (US); Alaina HOWE, Cambridge, MA (US); Dominick SALERNO, Cambridge, MA (US)

(21) Appl. No.: 19/476,020

(22) PCT Filed: Apr. 18, 2024
(86) PCT No.: PCT/US2024/025270

§ 371 Date: Oct. 16, 2025

Related U.S. Application Data
(60) Provisional application No. 63/460,389, filed on Apr. 19, 2023.
(60) Provisional application No. 63/514,497, filed on Jul. 19, 2023.
(60) Provisional application No. 63/520,809, filed on Aug. 21, 2023.
(60) Provisional application No. 63/596,901, filed on Nov. 7, 2023.

(51) Int. Cl. A61K 39/215; A61K 9/51; A61K 39/00; A61P 37/04

(52) U.S. Cl. CPC A61K 39/215; A61K 9/5123; A61P 37/04; A61K 2039/53

(57) ABSTRACT

Disclosed herein are nucleic acid vaccine compositions including one or more polynucleotides encoding one or more antigenic polypeptide, formulated within a lipid reconstructed natural messenger packs (LNMPs) comprising natural lipids and an ionizable lipid. The disclosure also includes a method for making a nucleic acid vaccine, comprising reconstituting a film comprising purified NMP lipids in the presence of an ionizable lipid to produce a LNMP comprising the ionizable lipid, and loading into the LNMPs with one or more polynucleotides encoding one or more antigenic polypeptides.

### CROSS-REFERENCE TO RELATED APPLICATIONS

This application claims benefit of priority to U.S. Provisional Application No. 63/460,389, filed Apr. 19, 2023; U.S. Provisional Application No. 63/514,497, filed Jul. 19, 2023; U.S. Provisional Application No. 63/520,809, filed Aug. 21, 2023; and U.S. Provisional Application No. 63/596,901, filed Nov. 7, 2023, all of which are herein incorporated by reference in their entirety.

### BACKGROUND

Newly emerging acute respiratory virus infections caused by novel coronavirus is a significant public health concern. The pandemic disease that the SARS-CoV-2 virus causes has been named by the World Health Organization (WHO) as COVID-19 (Coronavirus Disease 2019). The public health crisis caused by SARS-CoV-2 reinforces the importance of rapidly developing effective, easily scalable, and stable vaccine delivery against these viruses.

RNA vaccines have recently been showing great promise. A need therefore exists for developing an enhanced RNA delivery system for a more effective, easily scalable, and stable vaccine delivery.

### SUMMARY OF THE INVENTION

In one aspect, provided herein is a nucleic acid vaccine, comprising one or more polynucleotides encoding one or more antigenic polypeptides derived from an infectious agent that causes an infectious disease, disorder, or condition. The one or more polynucleotides are formulated within a complex lipid particle (CLP), such as a lipid reconstructed natural messenger packs (LNMPs) comprising natural lipids and an ionizable lipid. The ionizable lipid has two or more of the characteristics listed below:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heterorganic group separated by a chain of at least two atoms; and
(v) an N:P ratio of at least 3.

In another aspect, provided herein is a nucleic acid vaccine, comprising one or more polynucleotides encoding one or more antigenic polypeptides derived from an infectious agent that causes an infectious disease, disorder, or condition, formulated within a CLP such as a lipid reconstructed natural messenger pack (LNMP) comprising natural lipids and an ionizable lipid.

In another aspect, provided herein is a method for making a nucleic acid vaccine. The method comprises reconstituting a film comprising purified natural lipids in the presence of an ionizable lipid to produce a lipid reconstructed natural messenger pack (LNMP) comprising the ionizable lipid described herein. The method further comprises loading into the LNMPs with one or more polynucleotides encoding one or more antigenic polypeptides derived from an infectious agent that causes an infectious disease, disorder, or condition.

In these aspects of the invention, the ionizable lipid may be selected from one of the following groups of compounds:
i) a compound of formula

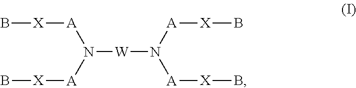

a pharmaceutically acceptable salt thereof, or a stereoisomer of any of the foregoing, wherein:
each A is independently C₁-C₁₆ branched or unbranched alkyl or C₁-C₁₆ branched or unbranched alkenyl, optionally substituted with heteroatom or substituted with OH, SH, or halogen;
each B is independently C₁-C₁₆ branched or unbranched alkyl or C₁-C₁₆ branched or unbranched alkenyl, optionally substituted with heteroatom or substituted with OH, SH, or halogen;
each X is independently a biodegradable moiety; and
W is

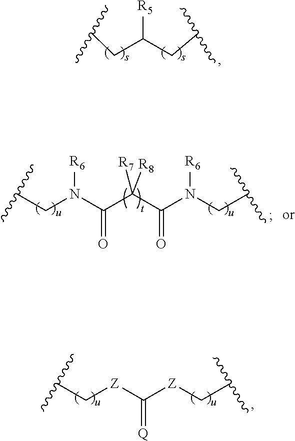

wherein:
   R₅ is OH, SH, or NR₁₀R₁₁;
   each R₆ is independently H, C₁-C₃ branched or unbranched alkyl, C₂-C₃ branched or unbranched alkenyl, or cycloalkyl;
   each R₇ and each R₈ is independently H, C₁-C₃ branched or unbranched alkyl, C₂-C₃ branched or unbranched alkenyl, halogen, OH, SH, or NR₁₀R₁₁, wherein each R₁₀ and R₁₁ is independently H, C₁-C₃ alkyl, or R₁₀ and R₁₁ are taken together to form a heterocyclic ring; R₇ and R₈ are taken together to form a ring;
   each s is independently 1, 2, 3, 4, or 5;
   each u is independently 1, 2, 3, 4, or 5;
   t is 1, 2, 3, 4 or 5;
   each Z is independently absent, O, S, or NR₁₂, wherein R₁₂ is H, C₁-C₇ branched or unbranched alkyl, or C₂-C₇ branched or unbranched alkenyl, and
   Q is O, S, or NR₁₃, wherein each R₁₃ is H, or C₁-C₅ alkyl;
ii) a compound of formula

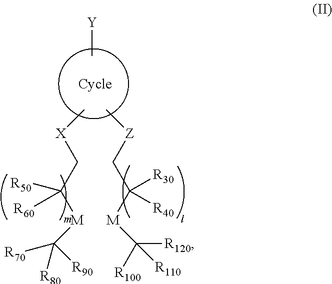

a pharmaceutically acceptable salt thereof, or a stereoisomer of any of the foregoing, wherein:

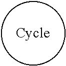

is a cyclic or heterocyclic moiety;
Y is alkyl, hydroxy, hydroxyalkyl or

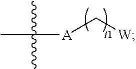

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each of X and Z is independently absent, —O—, —CO—, —N(R⁷)—, —O-alkylene-; -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, hydroxyalkyl, or aminoalkyl;
each M is independently a biodegradable moiety;
each of R₃₀, R₄₀, R₅₀, R₆₀, R₇₀, R₈₀, R₉₀, R₁₀₀, R₁₁₀, and R₁₂₀ is independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
each of l and m is an integer from 1 to 10;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl; and
iii) a compound of formula

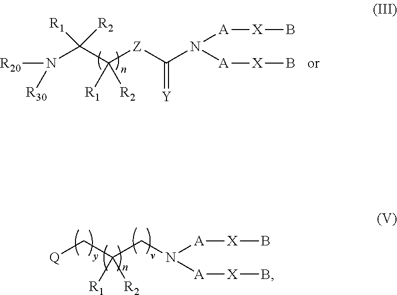

pharmaceutically acceptable salts thereof, and stereoisomers of any of the foregoing, wherein:
R₂₀ and R₃₀ are each independently H, C₁-C₅ branched or unbranched alkyl, or C₂-C₅ branched or unbranched alkenyl, or R₂₀ and R₃₀ together with the adjacent N atom form a 3 to 7 membered cyclic ring, optionally substituted with R^{a};
R^{a} is H, C₁-C₃ branched or unbranched alkyl, C₂-C₃ branched or unbranched alkenyl, halogen, OH, or SH;
each R₁ and each R₂ is independently H, C₁-C₃ branched or unbranched alkyl, C₂-C₃ branched or unbranched alkenyl, OH, halogen, SH, or NR₁₀R₁₁, or
R₁ and R₂ are taken together to form a cyclic ring;
each R₁₀ and R₁₁ is independently H, C₁-C₃ branched or unbranched alkyl, C₂-C₃ branched or unbranched alkenyl, or R₁₀ and R₁₁ are taken together to form a heterocyclic ring;
n is 0, 1, 2, 3 or 4;
Y is O or S;
Z is absent, O, S, or N(R₁₂), wherein each R₁₂ is independently H, C₁-C₇ branched or unbranched alkyl, or C₂-C₇ branched or unbranched alkenyl, provided that when Z is not absent, the adjacent R₁ and R₂ cannot be OH, NR₁₀R₁₁, or SH;
v is 0, 1, 2, 3, or 4;
y is 0, 1, 2, 3, or 4;
each A is each independently C₁-C₁₆ branched or unbranched alkyl, or C₂-C₁₆ branched or unbranched alkenyl, optionally interrupted with one or more heteroatoms or optionally substituted with OH, SH, or halogen;
each B is each independently C₁-C₁₆ branched or unbranched alkyl, or C₂-C₁₆ branched or unbranched alkenyl, optionally interrupted with one or more heteroatoms or optionally substituted with OH, SH, or halogen; and
each X is independently a biodegradable moiety; and
iv) a lipid comprising at least one head group and at least one tail group of formula (TI) or (TI′)

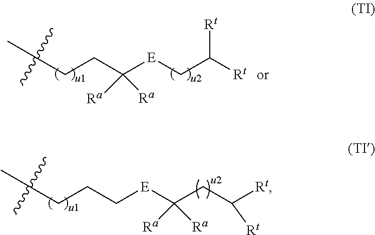

a pharmaceutically acceptable salt thereof, or a stereoisomer of any of the foregoing,
wherein:
E is each independently —OC(O)—, —C(O)O—, —N(R⁷)C(O)—, —C(O)N(R⁷)—, —C(O—R₁₃)—O—, —C(O)O(CH₂)ᵣ—, —C(O)N(R⁷)(CH₂)ᵣ—, —S—S—, or —C(O—R₁₃)—O—(CH₂)ᵣ—, wherein each R⁷ is independently H, alkyl, alkenyl, cycloalkyl, hydroxyalkyl, or aminoalkyl;
R₁₃ is branched or unbranched C₃-C₁₀ alkyl;
r is 1, 2, 3, 4, or 5;
R^{a} is each independently C₁-C₅ alkyl, C₂-C₅ alkenyl, or C₂-C₅ alkynyl;
u1 and u2 are each independently 0, 1, 2, 3, 4, 5, 6, or 7;
R^{t} is each independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
[symbol] represents the bond connecting the tail group to the head group; and
wherein the lipid has a pKa from about 4 to about 8.

In some embodiments, the ionizable lipid is a compound of group i), represented by a formula of

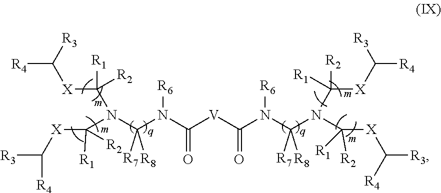

pharmaceutically acceptable salts thereof, and stereoisomers of any of the foregoing, wherein:
each R₁ and each R₂ is independently H, C₁-C₃ branched or unbranched alkyl, OH, halogen, SH, or NR₁₀R₁₁, or
each R₁ and each R₂ are independently taken together with the carbon atom(s) to which they are attached to form a cyclic ring;
each R₁₀ and R₁₁ is independently H, C₁-C₃ branched or unbranched alkyl, or R₁₀ and R₁₁ are taken together to form a heterocyclic ring;
each R₃ and each R₄ is independently H, C₂-C₁₄ branched or unbranched alkyl (e.g., C₃-C₁₀ branched or unbranched alkyl), or C₃-C₁₀ branched or unbranched alkenyl, provided that at least one of R₃ and R₄ is not H;
each X is independently a biodegradable moiety;
each q is independently 2, 3, 4, or 5;
V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups;
each R₆ is independently H, C₁-C₃ branched or unbranched alkyl, C₂-C₃ branched or unbranched alkenyl, or cycloalkyl;
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein each v is independently 0, 1, 2, 3, 4, or 5, and R₁₇ is OH, SH, or N(CH₃)₂; and
each m is independently 1, 2, 3, 4, 5, 6, 7, 8, 9 or 10. In some embodiments, V is a branched or unbranched C₂-C₃ alkylene, and each R₆ is independently H or methyl.

In some embodiments, the ionizable lipid is a compound of group i), represented by a formula of

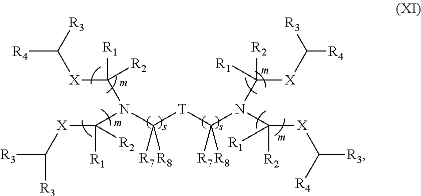

pharmaceutically acceptable salts thereof, and stereoisomers of any of the foregoing, wherein:
each R₁ and each R₂ is independently H, C₁-C₃ branched or unbranched alkyl, OH, halogen, SH, or NR₁₀R₁₁, or
each R₁ and each R₂ are independently taken together with the carbon atom(s) to which they are attached to form a cyclic ring;
each R₁₀ and R₁₁ is independently H, C₁-C₃ branched or unbranched alkyl, or R₁₀ and R₁₁ are taken together to form a heterocyclic ring;
each R₃ and each R₄ is independently H, C₂-C₁₄ branched or unbranched alkyl (e.g., C₃-C₁₀ branched or unbranched alkyl), or C₃-C₁₀ branched or unbranched alkenyl, provided that at least one of R₃ and R₄ is not H;
each X is independently a biodegradable moiety;
each s is independently 1, 2, 3, 4, or 5;
T is —NHC(O)O—, —OC(O)NH—, or a divalent heterocyclic optionally substituted with one or more —(CH₂)ᵥOH, —(CH₂)ᵥSH, —(CH₂)ᵥ-halogen groups,
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein R₁₇ is OH, SH, or N(CH₃)₂;
each v is independently 0, 1, 2, 3, 4, or 5; and
each m is independently 1, 2, 3, 4, 5, 6, 7, 8, 9 or 10. In some embodiments, T is a divalent piperazine or a divalent dioxopiperazine.

In some embodiments, in the above formulas for group i), X is —OCO—, —COO—, —NHCO—, or —CONH—.

In some embodiments, the ionizable lipid is a compound of group ii), represented by one of the following formulas:

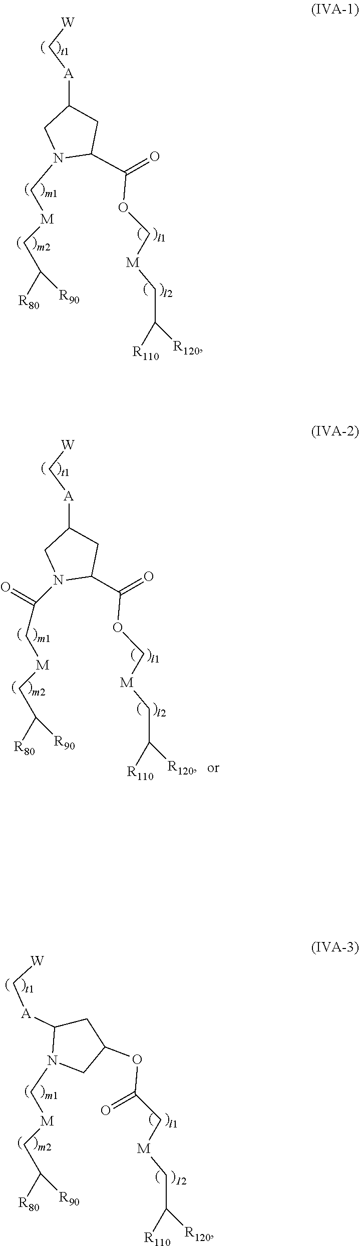

wherein:
A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each R⁷ is independently H, alkyl, alkenyl, cycloalkyl, hydroxy, hydroxyalkyl, or aminoalkyl;
t1 is an integer from 0 to 10;
W is hydroxyl, substituted or unsubstituted hydroxyalkyl, substituted or unsubstituted amino, substituted or unsubstituted aminocarbonyl, or substituted or unsubstituted heterocyclyl or heteroaryl;
each M is independently a biodegradable moiety;
each m1 is independently an integer from 3 to 6,
each l1 is independently an integer from 4 to 8,
m2 and l2 are each independently an integer from 0 to 3,
R₈₀ and R₉₀ are each independently unsubstituted C₅-C₈ alkyl or alkenyl; or R₈₀ is H or unsubstituted C₁-C₄ alkyl or alkenyl, and R₉₀ is unsubstituted C₅-C₁₁ alkyl or alkenyl; and
R₁₁₀ and R₁₂₀ are each independently unsubstituted C₅-C₈ alkyl or alkenyl; or R₁₁₀ is H or unsubstituted C₁-C₄ alkyl or alkenyl, and R₁₂₀ is unsubstituted C₅-C₁₁ alkyl or alkenyl. In some embodiments, M is —OC(O)— or —C(O)O—;

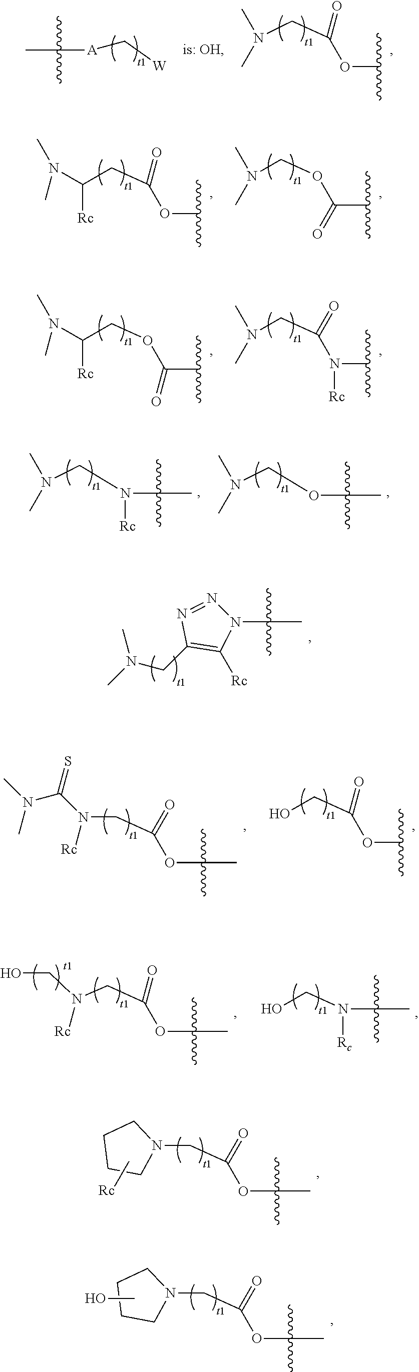

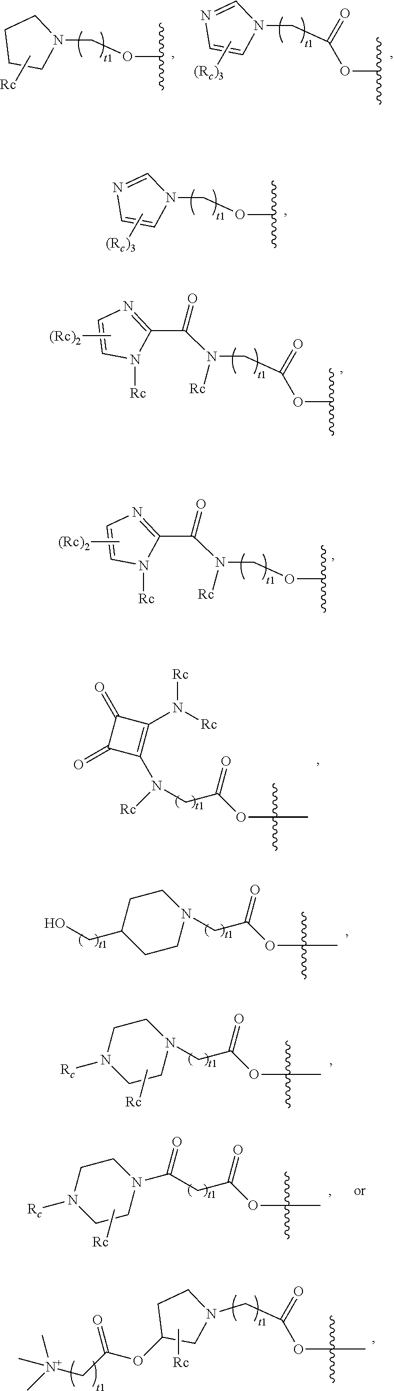

each R^{c} is independently H or C₁-C₃ alkyl; and
each t1 is independently 1, 2, 3, or 4.

In some embodiments, the ionizable lipid is a compound of group iii), wherein R₁ and R₂ are each H, or each R₁ is H, and one of the R₂ variables is OH; and X is —OC(O)— or —C(O)O—. In some embodiments, the ionizable lipid is a compound of group iii), represented by formula III), wherein R₂₀ and R₃₀ are each independently H or C₁-C₃ branched or unbranched alkyl; or R₂₀ and R₃₀ together with the adjacent N atom form a 3 to 7 membered cyclic ring, optionally substituted with R^{a}; R^{a} is H or OH; Z is absent, S, O, or NH; and n is 0, 1, or 2. In some embodiments, the ionizable lipid is a compound of group iii), represented by formula V),

In some embodiments, the ionizable lipid is a compound of group iv), wherein the lipid comprises at least one head group and at least one tail group, wherein:

the tail group has a structure of formula (TI) or formula TI′

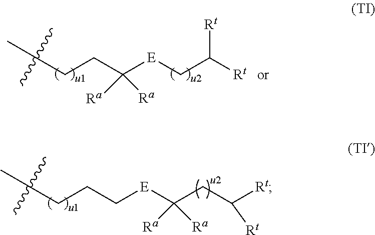

and the head group has a structure of one of the following formulas:

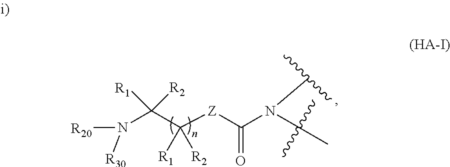

wherein:
   R₂₀ and R₃₀ are each independently H, C₁-C₅ branched or unbranched alkyl, or C₂-C₅ branched or unbranched alkenyl, optionally interrupted with one or more heteroatoms or substituted with OH, SH, halogen, or cycloalkyl groups; or
   R₂₀ and R₃₀, together with the adjacent N atom, form a 3 to 7 membered heterocyclic or heteroaromatic ring containing one or more heteroatoms, optionally substituted with one or more OH, SH, halogen, alkyl, or cycloalkyl groups;
   each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ together form a cyclic ring;
   each of R₁₀ and R₁₁ is independently H, C₁-C₃ branched or unbranched alkyl, C₂-C₃ branched or unbranched alkenyl; or R₁₀ and R₁₁ together form a heterocyclic ring;
   n is 0, 1, 2, 3 or 4; and
   Z is absent, O, S, or NR₁₂, wherein R₁₂ is H or C₁-C₇ branched or unbranched alkyl; provided that when Z is not absent, the adjacent R₁ and R₂ cannot be OH, NR₁₀R₁₁, or SH;

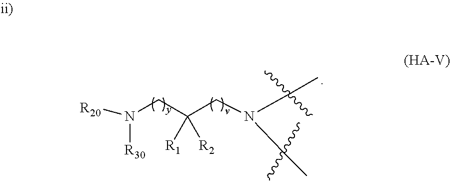

wherein:
   R₁ is H, C₁-C₃ alkyl, OH, halogen, SH, or NR₁₀R₁₁;
   R₂ is OH, halogen, SH, or NR₁₀R₁₁; or R₁ and R₂ can be taken together to form a cyclic ring;
   R₁₀ and R₁₁ are each independently H or C₁-C₃ alkyl; or R₁₀ and R₁₁ can be taken together to form a heterocyclic ring;
   R₂₀ and R₃₀ are each independently H, C₁-C₅ branched or unbranched alkyl, C₂-C₅ branched or unbranched alkenyl; or R₂₀ and R₃₀ can be taken together to form a cyclic ring; and
   each of v and y is independently 1, 2, 3, or 4;

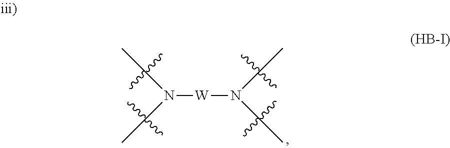

wherein W is

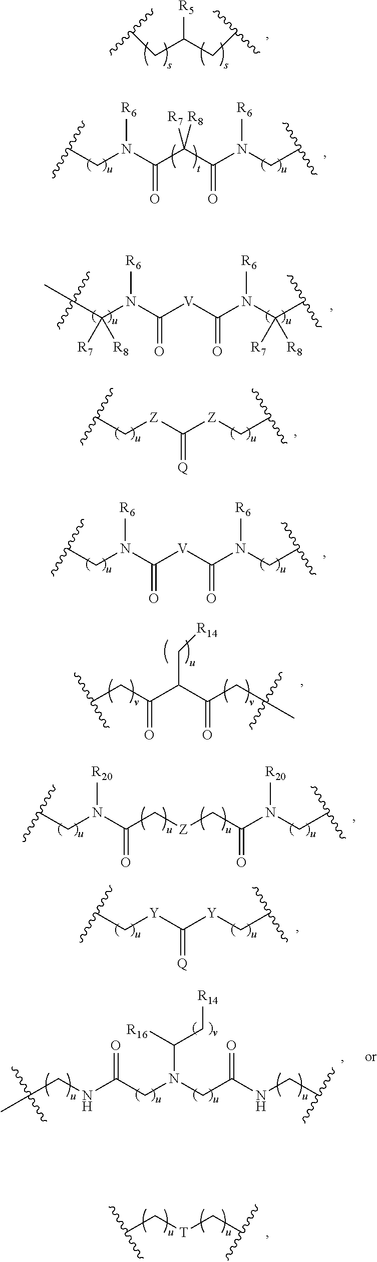

wherein
   R₅ is OH, SH, (CH₂)ₛOH, or NR₁₀R₁₁;
   each R₆ is independently H, C₁-C₃ branched or unbranched alkyl, C₂-C₃ branched or unbranched alkenyl, or cycloalkyl;
   each R₇ and R₈ are independently H, C₁-C₃ branched or unbranched alkyl, C₂-C₃ branched or unbranched alkenyl, halogen, (CH₂)ᵥOH, (CH₂)ᵥSH, (CH₂)ₛN(CH₃)₂, or NR₁₀R₁₁, wherein each R₁₀ and R₁₁ is independently H or C₁-C₃ alkyl, or R₁₀ and R₁₁ are taken together to form a heterocyclic ring; or R₇ and R₈ are taken together to form a ring;
   each R₂₀ is independently H, or C₁-C₃ branched or unbranched alkyl;
   R₁₄ is a heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H, C₁-C₃ alkyl, C₃-C₇ cycloalkyl, C₃-C₇ cycloalkenyl, optionally substituted with one or more NH and/or oxo groups, or R₁₀ and R₁₁ are taken together to form a heterocyclic ring;
   R₁₆ is H, ═O, ═S, or CN;
   each of s, u, and t is independently 1, 2, 3, 4, or 5;
   each v is independently 0, 1, 2, 3, 4, or 5;
   each Y is a divalent heterocyclic;
   each Z is independently absent, O, S, or NR₁₂, wherein R₁₂ is H, C₁-C₇ branched or unbranched alkyl, or C₂-C₇ branched or unbranched alkenyl;
   Q is O, S, CH₂, or NR₁₃, wherein each R₁₃ is H, or C₁-C₅ alkyl;
   V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups; and
   T is —NHC(O)O—, —OC(O)NH—, or a divalent heterocyclic; and

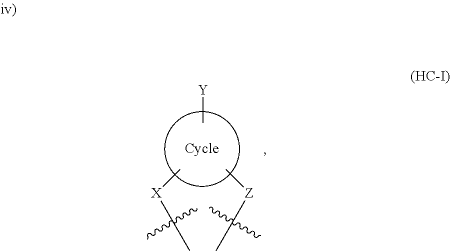

wherein:

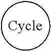

is cyclic or heterocyclic moiety;
   Y is alkyl, hydroxy, hydroxyalkyl,

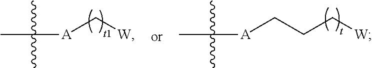

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, or —S—S—;
each of X and Z is independently absent, —O—, —C(O)—, —N(R⁷)—, —O-alkylene-, -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, alkoxy, hydroxyalkyl, alkylamino, alkylaminoalkyl, or aminoalkyl;
t is 0, 1, 2, or 3;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl; and
wherein the lipid has a pKa from about 4 to about 8.

In some embodiments, the ionizable lipid is a compound of group iv), and wherein at least one tail group of the lipid has one of the following formulas:

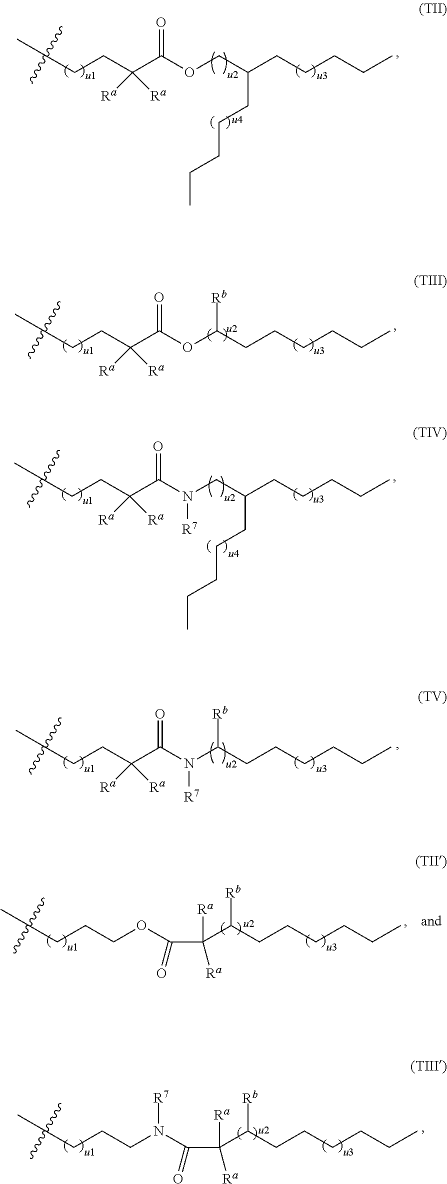

wherein:
R⁷ is each independently H or methyl;
R^{b} is in each occasion independently H or C₁-C₄ alkyl;
u1 and u2 are each independently 0, 1, 2, 3, 4, 5, 6, or 7; and
u3 and u4 are each independently 0, 1, 2, 3, 4, 5, 6, or 7; and
the head group has a structure of one of the following formulas:

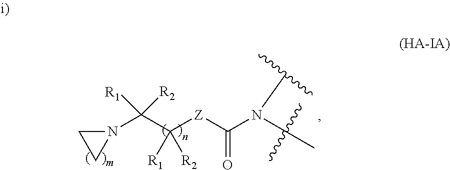

wherein m is 1, 2, 3, 4, 5, 6, 7 or 8;

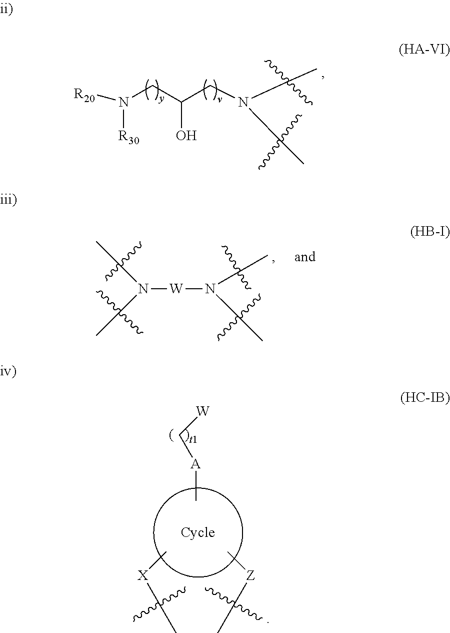

In some embodiments, at least one tail group has the structure of formula (TII), (TIII), (TIV), (TV), (TII′), and/or (TIII′), wherein u1 is 3-5, u2 is 0-3, u3 and u4 are each independently 1-7, and R^{a} is each independently methyl.

In some embodiments, the tail group has the structure of formula (TII) or formula (TIII), wherein each R^{a} is methyl; u1 is 3-5, u2 is 0-3; and u3 and u4 are each independently 1-4.

In some embodiments, the head group has the structure of one of the following formulas

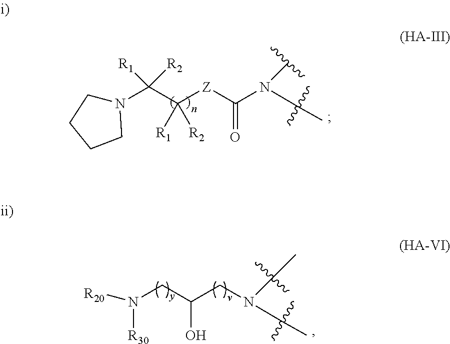

wherein each R₂₀ and R₃₀ are independently C₁-C₃ alkyl.

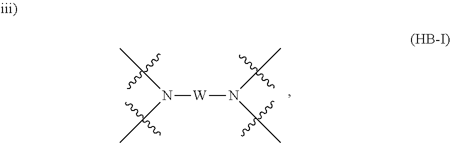

wherein:
W is

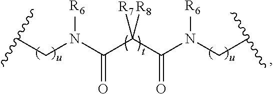

wherein:
   each R₆, R₇, and R₈ are independently H or methyl; and
   each of u and t is independently 1, 2, or 3; or
W is

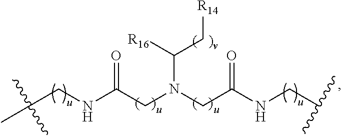

wherein:
   R₁₆ is H or ═O;
   R₁₄ is a nitrogen-containing 5- or 6-membered heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H or C₁-C₃ alkyl; and
   each of u and v is independently 1, 2, or 3; or
W is

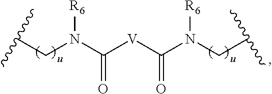

wherein:
   each R₆ is independently H or methyl;
   each u is independently 1, 2, or 3; and
   V is C₂-C₆ alkylene or C₂-C₆ alkenylene; or
W is

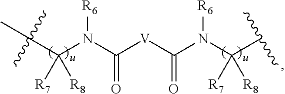

wherein:
   each R₆ is independently H or methyl;
   each R₇ is independently H;
   each R₈ is methyl;
   each u is independently 1, 2, or 3; and
   V is C₂-C₆ alkylene or C₂-C₆ alkenylene; or
W is

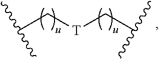

wherein:
   each u is independently 1, 2, or 3; and
   T is a divalent nitrogen-containing 5- or 6-membered heterocyclic; or
W is

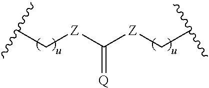

wherein:
each u is independently 1, 2, or 3;
Q is O;
each Z is independently NR₁₂; and
R₁₂ is H or C₁-C₃ alkyl; and

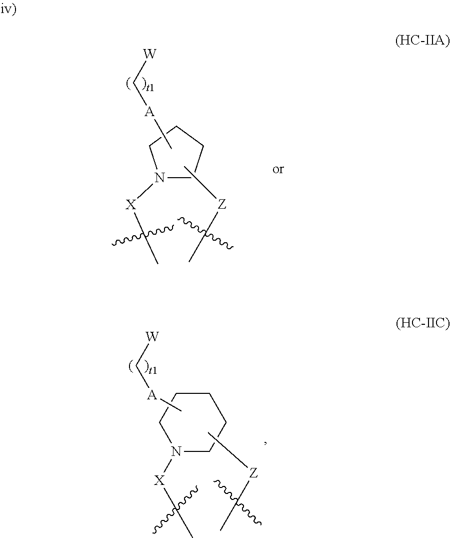

wherein:
W is hydroxyl, substituted or unsubstituted hydroxyalkyl, one of the following moieties:

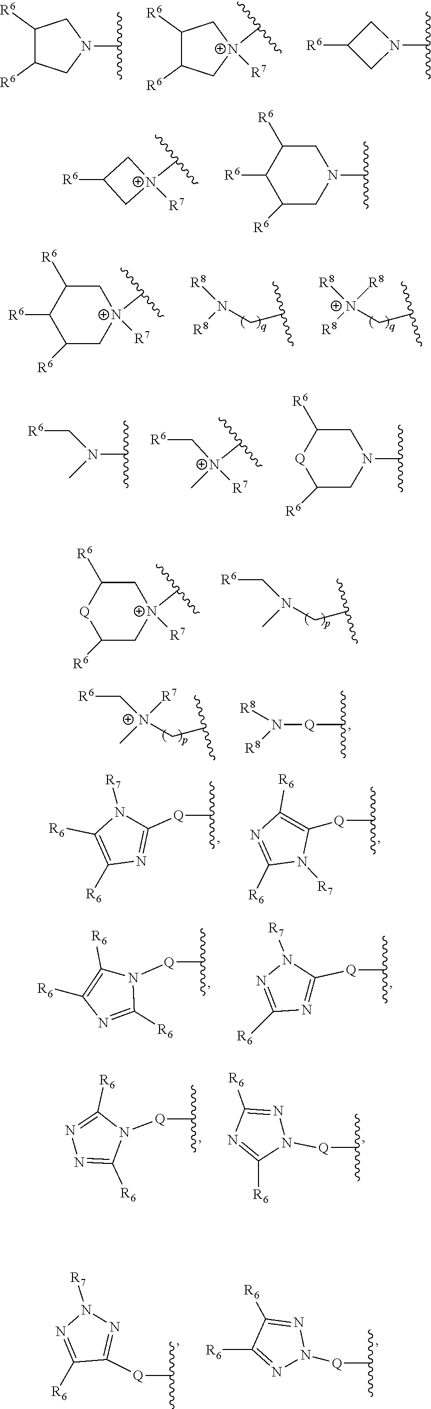

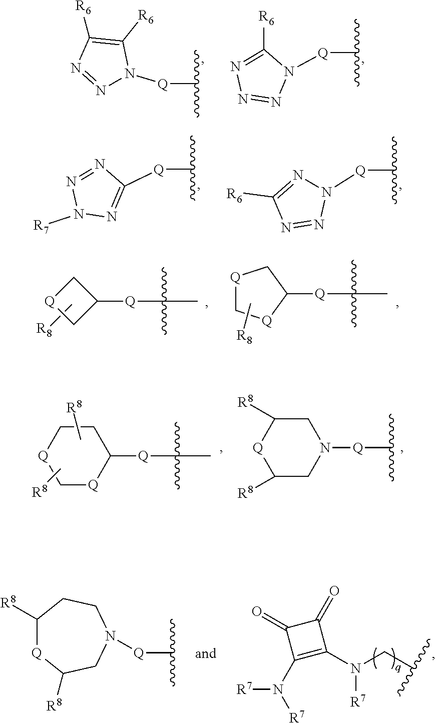

wherein
   each Q is independently absent, —O—, —C(O)—, —C(S)—, —C(O)O—, —(CH₂)_{q}—C(R⁷)₂—, —C(O)N(R⁷)—, —C(S)N(R⁷)—;
   R⁶ is independently H, alkyl, hydroxyl, hydroxyalkyl, alkoxy, —O-alkylene-O-alkyl,
—O-alkylene-N(R⁷)₂, amino, alkylamino, aminoalkyl, thiol, thiolalkyl, or N⁺(R⁷)₃-alkylene-Q-;
   each R⁸ is independently H, alkyl, hydroxyalkyl, amino, aminoalkyl, alkylamino, thiol, thiolalkyl, heterocyclyl, heteroaryl; or two R⁸ together with the nitrogen atom form a ring, optionally substituted with one or more alkyl, hydroxy, hydroxyalkyl, alkoxy, alkylaminoalkyl, alkylamino, or aminoalkyl;
   q is 0, 1, 2, 3, 4, or 5; and
   p is 0, 1, 2, 3, 4, or 5.

In some embodiments, the ionizable lipid is a compound in Table I, Table II, Table III, or Table IV.

In some embodiments, the ionizable lipid is Lipid No. 2275, 2272, 2248, 2308, 2335, 2425, or 2356.

In the CLP or LNMP formulations, more than one ionizable lipid can be used for the ionizable lipid component: one or more of the ionizable lipids from the compounds of formulas in groups i)-iv) can be used alone or in combination with a different ionizable lipid from the compounds of formulas in groups i)-iv).

In all these aspects of the invention, in some embodiments, the CLP is LNMP, and the CLP formulation encapsulating the one or more polynucleotides is a LNMP. Thus, all the embodiments below describing the features relating to LNMP and LNMP formulation are applicable to CLP and CLP formulation.

In some embodiments, the polynucleotides are polynucleotide constructs, which encode one or more wild type or engineered antigens (or an antibody to an antigen). The antigen may be derived from an infectious agent. In some embodiments, the infectious agent is a virus, e.g., a virus selected from the group consisting of an influenza virus, a corona virus, a mosquito-borne virus, a hepatitis virus, and an HIV virus. In some embodiments, the infectious agent is a virus, e.g., a respiratory syncytial virus, a rhinovirus, an adenovirus, or a parainfluenza virus. For instance, the infectious agent may be one or more strains of the viruses.

In some embodiments, the antigenic polypeptide encoded by the polynucleotide is a corona virus, or a fragment or subunit thereof. In some embodiments, the antigenic polypeptide is spike protein (S) of a MERS virus (MERS-CoV), a SARS virus (SARS-CoV), or a fragment or subunit thereof.

In some embodiments, the antigenic polypeptide is a SARS virus, or a fragment or subunit thereof. The antigenic polypeptide may be a SARS-CoV-2 spike protein or a SARS-CoV-2 spike glycoprotein. In one embodiment, the antigenic polypeptide is a wild-type SARS-CoV-2 spike glycoprotein.

In some embodiments, the polynucleotide may be a mRNA, an siRNA or siRNA precursor, a microRNA (miRNA) or miRNA precursor, a plasmid, a Dicer substrate small interfering RNA (dsiRNA), a short hairpin RNA (shRNA), an asymmetric interfering RNA (aiRNA), a peptide nucleic acid (PNA), a morpholino, a locked nucleic acid (LNA), a piwi-interacting RNA (piRNA), a ribozyme, a deoxyribozyme (DNAzyme), an aptamer, a circular RNA (circRNA), a guide RNA (gRNA), or a DNA molecule encoding any of these RNAs. In one embodiment, the polynucleotide is an mRNA. In one embodiment, the polynucleotide is a circRNA.

In some embodiments, the mRNA is derived from (a) a DNA molecule, or (b) an RNA molecule. In the mRNA, T is optionally substituted with U.

In some embodiments, the mRNA is derived from a DNA molecule. The DNA molecule can further comprise a promoter. In some embodiments, the promoter is a T7 promoter, a T3 promoter, or an SP6 promoter. In some embodiments, the promoter is located at the 5′ UTR.

In some embodiments, the mRNA is an RNA molecule. The RNA molecule may be a self-replicating RNA molecule.

In some embodiments, the mRNA is an RNA molecule. The RNA molecule may further comprise a 5′ cap. The 5′ cap can have a Cap 1 structure, a Cap 1 (m6A) structure, a Cap 2 structure, a Cap 3 structure, a Cap 0 structure, or any combination thereof.

In some embodiments, the mRNA comprises an open reading frame (ORF) that encodes a SARS-CoV-2 spike (S) glycoprotein having a double proline stabilizing mutation. In one embodiment, the double proline stabilizing mutation is at positions corresponding to K986 and V987 of a wild-type SARS-CoV-2 S glycoprotein.

In some embodiments, the mRNA comprises a 5′ untranslated region (UTR) and/or a 3′ UTR.

In some embodiments, the mRNA comprises a 5′ UTR. The 5′ UTR may comprise a Kozak sequence.

In some embodiments, the mRNA comprises a 3′ UTR. In some embodiments, the 3′ UTR comprises one or more sequences derived from an amino-terminal enhancer of split (AES). In some embodiments, the 3′ UTR comprises a sequence derived from mitochondrially encoded 12S rRNA (mtRNRI).

In some embodiments, the mRNA comprises a poly(A) sequence. In one embodiment, the poly(A) sequence is a 110-nucleotide sequence consisting of a sequence of 30 adenosine residues, a 10-nucleotide linker sequence, and a sequence of 70 adenosine residues.

In some embodiments, the polynucleotide comprises a circRNA represented by SEQ ID NO: 26 or a fragment thereof. In some embodiments, the polynucleotide comprises a linear RNA (a linear version of the circRNA represented by SEQ ID NO: 26) represented by SEQ ID NO: 27 or a fragment thereof.

In some embodiments, the polynucleotide is encapsulated by the lipid reconstructed natural messenger packs (LNMPs). In some embodiments, the polynucleotide is encapsulated by the lipid reconstructed plant messenger packs (LPMPs). In some embodiments, the polynucleotide is embedded on the surface of the LNMPs. In some embodiments, the polynucleotide is conjugated to the surface of the LNMPs.

In some embodiments, the LNMP is produced by a method comprising lipid extrusion. In some embodiments, the LNMP is produced by a method comprising processing a solution comprising a lipid extract of the NMPs in a microfluidics device comprising an aqueous phase, thereby producing the LNMPs. In some embodiments, the aqueous phase comprises the polynucleotides.

In some embodiments, the natural lipids of the LNMPs are extracted from a plant source, such as lemon or algae. In some embodiments, the natural lipids are soy-derived lipids. In some embodiments, the soy-derived lipids comprise soy PC, soy PE, soy PI, soy PA, lyso PC (soy LPC), lyso PI (soy LPI), soy PG, soyl PL (phospholipid) mixture, soy PS, soy LPS, soy polar, or a combination thereof. In some embodiments, the natural lipids are extracted from a bacteria source, such as E. coli or Salmonella typhimurium.

In the LNMP formulations, for the ionizable lipid component, the ionizable lipids from the compounds of formulas in groups i)-iv) can be used in combination with one or more other ionizable lipids. For instance, one or more other ionizable lipids can include 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315. In one embodiment, the additional ionizable lipid included is C12-200.

In some embodiments, the additional ionizable lipid included is

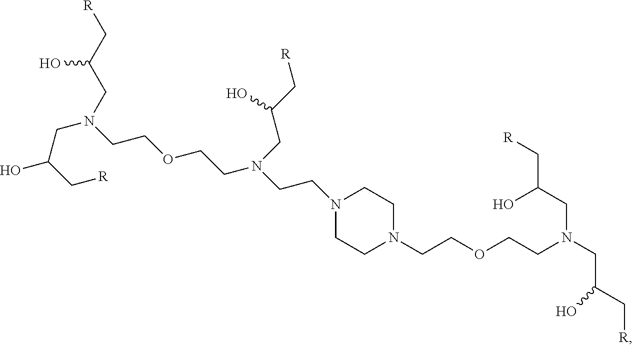

wherein R is C8-C14 alkyl group.

In some embodiments, the reconstitution is performed in the presence of a sterol, thereby producing a LNMP that comprises natural lipids, an ionizable lipid, and a sterol. In some embodiments, the sterol is cholesterol or sitosterol.

In some embodiments, the reconstitution is performed in the presence of a PEGylated lipid (or a PEG-lipid conjugate), thereby producing a LNMP that comprises natural lipids, the ionizable lipid, and a PEG-lipid conjugate. In some embodiments, the LNMPs further comprise a sterol and a polyethylene glycol (PEG)-lipid conjugate.

In some embodiments, the PEG-lipid conjugate is C14-PEG2k, C18-PEG2k, or DMPE-PEG2k. In some embodiments, the PEG-lipid conjugate is PEG-DMG or PEG-PE. In some embodiments, the PEG-DMG is PEG2000-DMG or PEG2000-PE.

In some embodiments, the LNMP comprises:
about 20 mol % to about 50 mol % of the ionizable lipid,
about 5 mol % to about 60 mol % of the natural lipids, and optionally a neutral lipid,
about 7 mol % to about 50 mol % of the sterol, and
about 0.5 mol % to about 3 mol % of the polyethylene glycol (PEG)-lipid conjugate.

In one embodiment, the LNMPs comprise the ionizable lipid:natural lipids:sterol:PEG-lipid at a molar ratio of about 35:50:12.5:2.5. In one embodiment, the LNMPs comprise the ionizable lipid:natural lipids:sterol:PEG-lipid at a molar ratio of about 35:20:42.5:2.5. In one embodiment, the LNMPs comprise the ionizable lipid:natural lipids:sterol:PEG-lipid at a molar ratio of about 45:20:33.5:2.5. In one embodiment, the LNMPs comprise the ionizable lipid:natural lipids:sterol:PEG-lipid at a molar ratio of about 45:10:43.5:1.5.

In some embodiments, the LNMP may further comprise a neutral lipid as a helper lipid. In some embodiments, the natural lipids may be used in combination with a neutral lipid as a structural lipid component. The neutral lipid may be used in a molar ratio of neutral lipid:natural lipid of 10:1 to 1:10, or 3:1 to 1:3, e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10. Non-limiting examples of neutral lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. In one embodiment, the structural lipid component in the LNMP may comprise a natural lipid+DOPE, or a natural lipid+DSPC.

In these embodiments, the LNMP thus comprises the ionizable lipids described herein, a structural lipid comprising natural lipids and a neutral lipid, a sterol and/or a PEG-lipid.

In some embodiments, the LNMP comprises:
about 20 mol % to about 50 mol % of the ionizable lipid,
about 5 mol % to about 60 mol % of a structural lipid component (i.e., the natural lipids and the neutral lipid),
about 7 mol % to about 50 mol % of the sterol, and
about 0.5 mol % to about 3 mol % of the polyethylene glycol (PEG)-lipid conjugate.

In one embodiment, the LNMPs comprise the ionizable lipid:(natural lipids+ neutral lipid):sterol:PEG-lipid at a molar ratio of about 35:50:12.5:2.5. In one embodiment, the LNMPs comprise the ionizable lipid:(natural lipids+ neutral lipid):sterol:PEG-lipid at a molar ratio of about 45:20:33.5:2.5. In one embodiment, the LNMPs comprise the ionizable lipid:(natural lipids+ neutral lipid):sterol:PEG-lipid at a molar ratio of about 45:10:43.5:1.5. In one embodiment, the LNMPs comprise the ionizable lipid:(natural lipids+ neutral lipid):sterol:PEG-lipid at a molar ratio of about 35:20:42.5:2.5. For instance, the LNMPs may comprise the ionizable lipid:(natural lipids+ neutral lipid):sterol:PEG-lipid at a molar ratio of about 35:(10+10):42.5:2.5. In one embodiment, the LNMPs comprise ionizable lipid:(natural lipids+ neutral lipid):sterol:PEG-lipid at a molar ratio of about 50:20:28.5:1.5. For instance, the LNMPs may comprise the ionizable lipid:(natural lipids+ neutral lipid):sterol:PEG-lipid at a molar ratio of about 50:(10+10):28.5:1.5.

In some embodiments, the LNMPs comprise:
natural lipids extracted from a plant source (such as soy, lemon, or algae), and optionally a neutral lipid,
the ionizable lipid from Table I, Table II, Table III, or Table IV,
cholesterol, and
DMG-PEG.

In some embodiments, the LNMPs comprise:
natural lipids extracted from lemon or algae, and optionally a neutral lipid,
the ionizable lipid from Table I, Table II, Table III, or Table IV,
cholesterol, and
DMPE-PEG2k.

In one embodiment, the LNMPs comprise:
natural lipids extracted from lemon, and optionally a neutral lipid,
the ionizable lipid from Table I, Table II, Table III, or Table IV,
cholesterol, and
DMG-PEG or DMPE-PEG2k. The LNMPs may comprise the ionizable lipid:lemon lipids:cholesterol:DMPE-PEG2 k at a molar ratio of about 35:50:12.5:2.5, about 35:20:42.5:2.5, about 50:20:28.5:1.5, about 45:20:33.5:2.5, or about 45:10:43.5:1.5. The LNMPs may comprise the ionizable lipid:(lemon lipids+ neutral lipid):cholesterol:DMPE-PEG2 k at a molar ratio of about 35:50:12.5:2.5, about 35:20:42.5:2.5, about 50:20:28.5:1.5, about 45:20:33.5:2.5, or about 45:10:43.5:1.5. The LNMPs may comprise the ionizable lipid:lemon lipids:cholesterol:DMG-PEG at a molar ratio of about 35:50:12.5:2.5, about 35:20:42.5:2.5, about 50:20:28.5:1.5, about 45:20:33.5:2.5, or about 45:10:43.5:1.5. The LNMPs may comprise the ionizable lipid:(lemon lipids+ neutral lipid):cholesterol:DMG-PEG at a molar ratio of about 35:50:12.5:2.5, about 35:20:42.5:2.5, about 50:20:28.5:1.5, about 45:20:33.5:2.5, or about 45:10:43.5:1.5.

In one embodiment, the LNMPs comprise:
natural lipids extracted from algae, and optionally a neutral lipid,
the ionizable lipid from Table I, Table II, Table III, or Table IV,
cholesterol, and
DMPE-PEG2k. The LNMPs may comprise the ionizable lipid:algae lipids:cholesterol:DMPE-PEG2 k at a molar ratio of about 35:20:42.5:2.5, about 35:20:42.5:2.5, about 50:20:28.5:1.5, about 45:20:33.5:2.5, or about 45:10:43.5:1.5. The LNMPs may comprise the ionizable lipid:(algae lipids+ neutral lipid):cholesterol:DMPE-PEG2 k at a molar ratio of about 35:20:42.5:2.5, about 35:20:42.5:2.5, about 50:20:28.5:1.5, about 45:20:33.5:2.5, or about 45:10:43.5:1.5.

In one embodiment, the LNMPs comprise:
natural lipids extracted from algae, and optionally a neutral lipid,
the ionizable lipid from Table I, Table II, Table III, or Table IV,
cholesterol, and
DMG-PEG. The LNMPs may comprise the ionizable lipid:algae lipids:cholesterol:DMG-PEG at a molar ratio of about 35:20:42.5:2.5, about 35:20:42.5:2.5, about 50:20:28.5:1.5, about 45:20:33.5:2.5, or about 45:10:43.5:1.5. The LNMPs may comprise the ionizable lipid:(algae lipids+ neutral lipid):cholesterol:DMG-PEG at a molar ratio of about 35:20:42.5:2.5, about 35:20:42.5:2.5, about 50:20:28.5:1.5, about 45:20:33.5:2.5, or about 45:10:43.5:1.5.

In one embodiment, the LNMPs comprise:
natural lipids that are soy-derived lipids, and optionally a neutral lipid,
the ionizable lipid from Table I, Table II, Table III, or Table IV,
cholesterol, and
DMPE-PEG2k. The LNMPs may comprise the ionizable lipid:soy-derived lipids:cholesterol:DMPE-PEG2 k at a molar ratio of about 35:20:42.5:2.5, about 35:20:42.5:2.5, about 50:20:28.5:1.5, about 45:20:33.5:2.5, or about 45:10:43.5:1.5. The LNMPs may comprise the ionizable lipid:(soy-derived lipids+ neutral lipid):cholesterol:DMPE-PEG2 k at a molar ratio of about 35:20:42.5:2.5, about 35:20:42.5:2.5, about 50:20:28.5:1.5, about 45:20:33.5:2.5, or about 45:10:43.5:1.5.

In one embodiment, the LNMPs comprise:
natural lipids that are soy-derived lipids, and optionally a neutral lipid,
the ionizable lipid from Table I, Table II, Table III, or Table IV,
cholesterol, and
DMG-PEG. The LNMPs may comprise the ionizable lipid:soy-derived lipids:cholesterol:DMG-PEG at a molar ratio of about 35:20:42.5:2.5, about 35:20:42.5:2.5, or about 50:20:28.5:1.5. The LNMPs may comprise the ionizable lipid:(soy-derived lipids+ neutral lipid):cholesterol:DMG-PEG at a molar ratio of about 35:20:42.5:2.5, about 35:20:42.5:2.5, about 50:20:28.5:1.5, about 45:20:33.5:2.5, or about 45:10:43.5:1.5.

In some embodiments, the LNMPs comprise:
natural lipids extracted from E. coli or Salmonella typhimurium, and optionally a neutral lipid,
the ionizable lipid from Table I, Table II, Table III, or Table IV,
cholesterol, and
DMPE-PEG2k.

In one embodiment, the LNMPs comprise:
natural lipids extracted from E. coli, and optionally a neutral lipid,
the ionizable lipid from Table I, Table II, Table III, or Table IV,
cholesterol, and
DMPE-PEG2k. The LNMPs may comprise the ionizable lipid: E. coli lipids:cholesterol:DMPE-PEG2 k at a molar ratio of about 35:50:12.5:2.5, about 35:20:42.5:2.5, about 50:20:28.5:1.5, about 45:20:33.5:2.5, or about 45:10:43.5:1.5. The LNMPs may comprise ionizable lipid:(E. coli lipids+ neutral lipid):cholesterol:DMPE-PEG2 k at a molar ratio of about 35:50:12.5:2.5, about 35:20:42.5:2.5, about 50:20:28.5:1.5, about 45:20:33.5:2.5, or about 45:10:43.5:1.5.

In one embodiment, the LNMPs comprise:
natural lipids extracted from Salmonella typhimurium, and optionally a neutral lipid,
the ionizable lipid from Table I, Table II, Table III, or Table IV,
cholesterol, and
DMPE-PEG2k. The LNMPs may comprise the ionizable lipid: Salmonella typhimurium lipids:cholesterol:DMPE-PEG2 k at a molar ratio of about 35:20:42.5:2.5, about 35:20:42.5:2.5, about 50:20:28.5:1.5, about 45:20:33.5:2.5, or about 45:10:43.5:1.5. The LNMPs may comprise ionizable lipid:(Salmonella typhimurium lipids+ neutral lipid):cholesterol:DMPE-PEG2 k at a molar ratio of about 35:20:42.5:2.5, about 35:20:42.5:2.5, about 50:20:28.5:1.5, about 45:20:33.5:2.5, or about 45:10:43.5:1.5.

In some embodiments, the LNMPs comprise:
natural lipids extracted from E. coli or Salmonella typhimurium, and optionally a neutral lipid,
the ionizable lipid from Table I, Table II, Table III, or Table IV,
cholesterol, and
DMG-PEG.

In one embodiment, the LNMPs comprise:
natural lipids extracted from E. coli, and optionally a neutral lipid,
the ionizable lipid from Table I, Table II, Table III, or Table IV,
cholesterol, and
DMG-PEG. The LNMPs may comprise the ionizable lipid: E. coli lipids:cholesterol:DMG-PEG at a molar ratio of about 35:50:12.5:2.5, about 35:20:42.5:2.5, about 50:20:28.5:1.5, about 45:20:33.5:2.5, or about 45:10:43.5:1.5. The LNMPs may comprise ionizable lipid:(E. coli lipids+ neutral lipid):cholesterol:DMG-PEG at a molar ratio of about 35:50:12.5:2.5, about 35:20:42.5:2.5, about 50:20:28.5:1.5, about 45:20:33.5:2.5, or about 45:10:43.5:1.5.

In one embodiment, the LNMPs comprise:
natural lipids extracted from Salmonella typhimurium, and optionally a neutral lipid,
the ionizable lipid from Table I, Table II, Table III, or Table IV,
cholesterol, and
DMG-PEG. The LNMPs may comprise the ionizable lipid: Salmonella typhimurium lipids:cholesterol:DMG-PEG at a molar ratio of about 35:20:42.5:2.5, about 35:20:42.5:2.5, about 50:20:28.5:1.5, about 45:20:33.5:2.5, or about 45:10:43.5:1.5. The LNMPs may comprise ionizable lipid:(Salmonella typhimurium lipids+ neutral lipid):cholesterol:DMG-PEG at a molar ratio of about 35:20:42.5:2.5, about 35:20:42.5:2.5, about 50:20:28.5:1.5, about 45:20:33.5:2.5, or about 45:10:43.5:1.5.

In some embodiments, the LNMP is a lipophilic moiety selected from the group consisting of a lipoplex, a liposome, a lipid nanoparticle, a polymer-based carrier, an exosome, a lamellar body, a micelle, and an emulsion. In one embodiment, the LNMP is a liposome selected from the group consisting of a cationic liposome, a nanoliposome, a proteoliposome, a unilamellar liposome, a multilamellar liposome, a ceramide-containing nanoliposome, and a multivesicular liposome. In one embodiment, the LNMP is a lipid nanoparticle.

In some embodiments, the LNMP has a size of less than about 200 nm. In one embodiment, the LNMP has a size of less than about 150 nm. In one embodiment, the LNMP has a size of less than about 100 nm. In one embodiment, the LNMP has a size of about 80 nm to about 100 nm. In one embodiment, the LNMP has a size of about 55 nm to about 80 nm.

In some embodiments, the LNMP has an N:P ratio of at least 3, for instance, an N:P ratio of 3 to 100, 3 to 50, 3 to 30, 3 to 20, 3 to 15, 3 to 12, 6 to 30, 6 to 20, 6 to 15, or 6 to 12.

In some embodiments, the nucleic acid vaccine has a total lipid:polynucleotide weight ratio of about 50:1 to about 10:1. In one embodiment, the nucleic acid vaccine has a total lipid:polynucleotide weight ratio of about 44:1 to about 24:1. In one embodiment, the nucleic acid vaccine has a total lipid:polynucleotide weight ratio of about 40:1 to about 28:1. In one embodiment, the nucleic acid vaccine has a total lipid:polynucleotide weight ratio of about 38:1 to about 30:1. In one embodiment, the nucleic acid vaccine has a total lipid:polynucleotide weight ratio of about 37:1 to about 33:1.

In some embodiments, the nucleic acid vaccine, e.g., the aqueous phase further comprises a HEPES or TRIS buffer. The HEPES or TRIS buffer may have a pH of about 7.0 to about 8.5. The HEPES or TRIS buffer can be at a concentration of about 7 mg/mL to about 15 mg/mL. The aqueous phase may further comprise about 2.0 mg/mL to about 4.0 mg/mL of NaCl.

In some embodiments, the nucleic acid vaccine, e.g., the aqueous phase comprises water, PBS, or a citrate buffer. In one embodiment, the aqueous phase comprises a citrate buffer having a pH of about 3.2.

In some embodiments, the aqueous phase and the lipid solution are mixed at a 3:1 volumetric ratio.

In some embodiments, the nucleic acid vaccine further comprises one or more cryoprotectants. The one or more cryoprotectants may be sucrose, glycerol, or a combination thereof. In one embodiment, the nucleic acid vaccine comprises a combination of sucrose at a concentration of about 70 mg/mL to about 110 mg/mL and glycerol at a concentration of about 50 mg/mL to about 70 mg/mL.

In some embodiments, the nucleic acid vaccine is a lyophilized composition. The lyophilized nucleic acid vaccine may comprise one or more lyoprotectants. The lyophilized nucleic acid vaccine may comprise a poloxamer, potassium sorbate, sucrose, or any combination thereof. In one embodiment, the lyophilized nucleic acid vaccine comprises a poloxamer, e.g., poloxamer 188.

In some embodiments, the nucleic acid vaccine is a lyophilized composition. In one embodiment, the lyophilized nucleic acid vaccine comprises about 0.01 to about 1.0% w/w of the polynucleotides. In one embodiment, the lyophilized nucleic acid vaccine comprises about 1.0 to about 5.0% w/w lipids. In one embodiment, the lyophilized nucleic acid vaccine comprises about 0.5 to about 2.5% w/w of TRIS buffer. In one embodiment, the lyophilized nucleic acid vaccine comprises about 0.75 to about 2.75% w/w of NaCl. In one embodiment, the lyophilized nucleic acid vaccine comprises about 85 to about 95% w/w of a sugar, e.g., sucrose. In one embodiment, the lyophilized nucleic acid vaccine comprises about 0.01 to about 1.0% w/w of a poloxamer, e.g., poloxamer 188. In one embodiment, the lyophilized nucleic acid vaccine comprises about 1.0 to about 5.0% w/w of potassium sorbate.

In some embodiments, the nucleic acid vaccine induces germinal center formation. In some embodiments, the nucleic acid vaccine induces B cell class switching.

In some embodiments, the nucleic acid vaccine has lower systemic exposure. In certain embodiments, the vaccinated host exhibits lower liver expression of the encoded RNA (e.g., mRNA or circRNA). In some embodiments, the nucleic acid vaccine induces at least 2%, 5%, 10%, 15%, 20%, 25%, 30%, 35%, or 40% lower liver expression than a control lipid nanoparticle composition that (a) does not contain natural lipids and/or (b) does not contain the ionizable lipid selected from the compounds of formulas in groups i)-iv) (e.g., not the ionizable lipid selected from Table I, Table II, Table III, or Table IV). In other embodiments, the vaccinated host with the nucleic acid vaccine maintains spleen expression. In some embodiments, the nucleic acid vaccine maintains a spleen expression at a level comparable to or higher than a control lipid nanoparticle composition that (a) does not contain natural lipids and/or (b) does not contain the ionizable lipid selected from the compounds of formulas in groups i)-iv) (e.g., not the ionizable lipid selected from Table I, Table II, Table III, or Table IV). In some embodiments, the nucleic acid vaccine induces a number of antigen-specific T cells in spleen cells at least comparable to, or 2%, 5%, 10%, 15%, 20%, 25%, 30%, 35%, 40%, 45%, 50%, 55%, 60%, 65%, 70%, 75%, 80%, 85%, 90%, 95%, or 100% higher than a lipid nanoparticle composition that (a) does not contain natural lipids and/or (b) does not contain the ionizable lipid selected from the compounds of formulas in groups i)-iv) (e.g., not the ionizable lipid selected from Table I, Table II, Table III, or Table IV).

In some embodiments, the nucleic acid vaccine reduces detectable infectious particles in the nares compared to an unvaccinated control. In other embodiments, the nucleic acid vaccine reduces detectable infectious particles in the lungs compared to an unvaccinated control. In certain embodiments, the detectable infectious particles are reduced 2-fold, 3-fold, 5-fold, 10-fold, 20-fold, 30-fold, or more compared to an unvaccinated control.

Aspects of the invention also provide for methods of preventing or reducing the transmission of an infectious disease, disorder, or condition. The method comprises administering to a subject the nucleic acid vaccine described in the above aspects of the invention, thereby preventing or reducing the transmission of an infectious disease, disorder, or condition. In some embodiments, the method reduces the transmission of an infectious disease, disorder, or condition by at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%). Alternatively, the method comprises administering to a subject the nucleic acid vaccine described in the above aspects of the invention, thereby reducing the transmission level of an infectious disease, disorder, or condition to another subject, to less than 10% (e.g., less than 5%, less than 4%, less than 3%, less than 2%, less than 1%, less than 0.5%, less than 0.4%, less than 0.3%, less than 0.2%, or less than 0.1%). In some embodiments, the method prevents or reduces the transmission of the infectious agent from a vaccinated host to an unvaccinated host. In some embodiments, the method prevents or reduces the transmission of the infectious agent from a vaccinated host to a vaccinated host.

### Definitions

As used herein, the term “effective amount,” “effective concentration,” or “concentration effective to” refers to an amount of a LNMP (e.g., LPMP), or nucleic acid composition, sufficient to effect the recited result or to reach a target level (e.g., a predetermined or threshold level) in or on a target organism.

As used herein, the term “therapeutic agent” refers to an agent that can act on an animal, e.g., a mammal (e.g., a human), an animal pathogen, or a pathogen vector, such as an antifungal agent, an antibacterial agent, a virucidal agent, an anti-viral agent, an insecticidal agent, a nematicidal agent, an antiparasitic agent, or an insect repellent.

As defined herein, the term “nucleic acid” and “polynucleotide” are interchangeable and refer to RNA or DNA that is linear or branched, single or double stranded, or a hybrid thereof, regardless of length (e.g., at least 2, 3, 4, 5, 6, 7, 8, 9, 10, 20, 30, 40, 50, 100, 150, 200, 250, 500, 1000, or more nucleic acids). The term also encompasses RNA/DNA hybrids. Nucleotides are typically linked in a nucleic acid by phosphodiester bonds, although the term “nucleic acid” also encompasses nucleic acid analogs having other types of linkages or backbones (e.g., phosphoramide, phosphorothioate, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, among others). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases (including, e.g., hypoxanthine, xanthine, 7-methylguanine, 5,6-dihydrouracil, 5-methylcytosine, and 5 hydroxymethylcytosine).

As used herein, the terms “circRNA,” “circular polyribonucleotide,” “circular RNA,” and “circular polyribonucleotide molecule” are used interchangeably and mean a polyribonucleotide molecule that has a structure having no free ends (i.e., no free 3′ and/or 5′ ends), for example a polyribonucleotide molecule that forms a circular or end-less structure through covalent (e.g., covalently closed) or non-covalent bonds. The circular polyribonucleotide may be, e.g., a covalently closed polyribonucleotide.

As used herein, the term “expression sequence” is a nucleic acid sequence that encodes a product, e.g., a polypeptide or a regulatory nucleic acid. An exemplary expression sequence that codes for a polypeptide can comprise a plurality of nucleotide triads, each of which can code for an amino acid and is termed as a “codon”.

As used herein, the terms “linear RNA,” “linear polyribonucleotide,” and “linear polyribonucleotide molecule” are used interchangeably and mean a monoribonucleotide molecule or polyribonucleotide molecule having a 5′ and 3′ end. One or both of the 5′ and 3′ ends may be free ends or joined to another moiety. In some embodiments, the linear RNA has a 5′ end or 3′ end that is modified or protected from degradation (e.g., by a 5′ end protectant or a 3′ end protectant). In some embodiments, the linear RNA has non-covalently linked 5′ or 3′ ends. A linear RNA can be used as a starting material for circularization through, for example, splint ligation, or chemical, enzymatic, ribozyme- or splicing-catalyzed circularization methods.

As used herein, the term “polyribonucleotide cargo” herein includes any sequence including at least one polyribonucleotide. In embodiments, the polyribonucleotide cargo includes one or multiple expression sequences, wherein each expression sequence encodes a polypeptide. In embodiments, the polyribonucleotide cargo includes one or multiple noncoding sequences, such as a polyribonucleotide having regulatory or catalytic functions. In embodiments, the polyribonucleotide cargo includes a combination of expression and noncoding sequences. In embodiments, the polyribonucleotide cargo includes one or more polyribonucleotide sequence described herein, such as one or multiple regulatory elements, internal ribosomal entry site (IRES) elements, or spacer sequences.

As used herein, the elements of a nucleic acid are “operably connected” or “operably linked” if they are positioned on the vector such that they can be transcribed to form a linear RNA that can then be circularized into a circular RNA using the methods provided herein.

As used herein, a “spacer” or “spacer sequence” refers to any contiguous, non-coding nucleotide sequence (e.g., of one or more nucleotides) that provides distance or flexibility between two adjacent polynucleotide regions. Exemplary spacer sequences include, but are not limited to, poly(X) sequences as described herein, repetitive or random non-coding DNA or RNA sequences located 3′ or 5′ to open reading frames, or 3′ or 5′ untranslated regions. Any spacer sequence deemed appropriate by the skilled artisan for the polyribonucleotides described herein are contemplated by this disclosure.

As used interchangeably herein, the terms “poly(X)” and “poly(X) sequence” refer to an untranslated, contiguous region of any nucleic acid molecule of at least 5 nucleotides in length and consisting of individual adenine (A), thymine (T), cytosine (C), guanine (G), or uracil (U) residues, or some combination thereof. For example, in some embodiments, a poly(A) sequence may be sequence of adenine residues. In other embodiments, a poly(A-T) sequence is a combination of adenine and thymine residues. In other embodiments, a poly(A-U) sequence may be a combination of adenine and uracil residues. In some embodiments, a poly(A-G) sequence is a combination of adenine and guanine residues. In some embodiments, a poly(G-C) sequence is a combination of guanine and cytosine residues. In some embodiments, a poly(X) sequence may be at least about 50 nucleotides to about 700 nucleotides in length, at least about 60 nucleotides to about 600 nucleotides in length, at least about 70 nucleotides to about 500 nucleotides in length, at least about 80 nucleotides to about 400 nucleotides in length, at least about 90 nucleotides to about 300 nucleotides in length, at least about 100 nucleotides to about 200 nucleotides in length. In some embodiments, the poly(X) sequence may be at least about 50, at least about 100, at least about 200, at least about 300, at least about 400, at least about 500, at least about 600, or at least about 700 nucleotides in length. In some embodiments, a poly(X) sequence may be located 3′ to (e.g., downstream of) an open reading frame (e.g., an open reading frame encoding a polypeptide), and the poly(X) sequence may be 3′ to a termination element (e.g., a stop codon) such that the poly(X) sequence is not translated. In some embodiments, a poly(X) sequence may be located 3′ to a termination element and a 3′ spacer sequence.

As used herein, the terms “nicked RNA,” “nicked linear polyribonucleotide,” and “nicked linear polyribonucleotide molecule” are used interchangeably and mean a polyribonucleotide molecule having a 5′ and 3′ end that results from nicking or degradation of a circular RNA.

As used herein, the term “peptide,” “protein,” or “polypeptide” encompasses any chain of naturally or non-naturally occurring amino acids (either D- or L-amino acids), regardless of length (e.g., at least 2, 3, 4, 5, 6, 7, 10, 12, 14, 16, 18, 20, 25, 30, 40, 50, 100, 150, 200, 250, 300, 350, 400, 450, 500, 550, 600, 650, 700, 750, 800, 850, 900, 950, 1000, or more than 1000 amino acids), the presence or absence of post-translational modifications (e.g., glycosylation or phosphorylation), or the presence of, e.g., one or more non-amino acyl groups (for example, sugar, lipid, etc.) covalently linked to the peptide, and includes, for example, natural proteins, synthetic, or recombinant polypeptides and peptides, hybrid molecules, peptoids, or peptidomimetics. The polypeptide may be, e.g., at least 0.1, at least 1, at least 5, at least 10, at least 15, at least 20, at least 30, at least 40, at least 50, or more than 50 kD in size. The polypeptide may be a full-length protein. Alternatively, the polypeptide may comprise one or more domains of a protein.

As used herein, the term “animal” refers to humans and non-human animals (including for example, dogs, cats, horses, rabbits, zoo animals, cows, pigs, sheep, chickens, and non-human primates).

As used herein, the term “infection” refers to the presence or colonization of a pathogen in an animal (e.g., in one or more parts of the animal), on an animal (e.g., on one or more parts of the animal), or in the habitat surrounding an animal, particularly where the infection decreases the fitness of the animal, e.g., by causing a disease, disease symptoms, or an immune (e.g., inflammatory) response.

As used herein the term “pathogen” refers to an organism, such as a microorganism or an invertebrate, which causes disease or disease symptoms in an animal by, e.g., (i) directly infecting the animal, (ii) producing agents that causes disease or disease symptoms in an animal (e.g., bacteria that produce pathogenic toxins and the like), and/or (iii) by eliciting an immune (e.g., inflammatory response) in animals (e.g., biting insects, e.g., bedbugs). As used herein, pathogens include, but are not limited to, bacteria, protozoa, parasites, fungi, nematodes, insects, viroids and viruses, or any combination thereof, wherein each pathogen is capable, either by itself or in concert with another pathogen, of eliciting disease or symptoms in humans.

As used herein, the term “antibody” encompasses an immunoglobulin, whether natural or partly or wholly synthetically produced, and fragments thereof, capable of specifically binding to an antigen. The term also covers any protein having a binding domain which is homologous to an immunoglobulin binding domain. These proteins can be derived from natural sources, or partly or wholly synthetically produced. “Antibody” further includes a polypeptide comprising a framework region from an immunoglobulin gene or fragments thereof that specifically binds and recognizes an antigen. Use of the term “antibody” is meant to include whole antibodies; polyclonal, monoclonal and recombinant antibodies; fragments thereof; and further includes single-chain antibodies (nanobodies); humanized antibodies; murine antibodies; chimeric, mouse-human, mouse-primate, primate-human monoclonal antibodies; anti-idiotype antibodies; antibody fragments, such as, e.g., scFv, (scFv)2, Fab, Fab′, and F(ab′)2, F(ab1)2, Fv, dAb, and Fd fragments; diabodies; and antibody-related polypeptides. “Antibody” further includes bispecific antibodies and multispecific antibodies.

As used herein, the term “heterologous” refers to an agent (e.g., a polypeptide) that is either (1) exogenous to the plant (e.g., originating from a source that is not the plant or plant part from which the PMP is produced) (e.g., an agent which is added to the PMP using loading approaches described herein) or (2) endogenous to the plant cell or tissue from which the PMP is produced, but present in the PMP (e.g., added to the PMP using loading approaches described herein, genetic engineering, as well as in vitro or in vivo approaches) at a concentration that is higher than that found in nature (e.g., higher than a concentration found in a naturally-occurring plant extracellular vesicle).

As used herein, “percent identity” between two sequences is determined by the BLAST 2.0 algorithm, which is described in Altschul et al., (1990) J. Mol. Biol. 215:403-410. Software for performing BLAST analyses is publicly available through the National Center for Biotechnology Information.

As used herein, the term “modified NMPs” or “modified LNMPs” refers to a composition including a plurality of NMPs or LNMPs that include one or more heterologous agents (e.g., one or more exogenous lipids, such as a ionizable lipids, e.g., a NMP or LNMP comprising an ionizable lipid and a sterol and/or a PEGylated lipid) capable of increasing cell uptake (e.g., animal cell uptake, plant cell uptake, bacterial cell uptake, or fungal cell uptake) of the NMP or LNMP, or a portion or component thereof, relative to an unmodified NMP or LNMP; capable of enabling or increasing delivery of a heterologous functional agent (e.g., an agricultural or therapeutic agent) by the NMP or LNMP to a cell, and/or capable of enabling or increasing loading (e.g., loading efficiency or loading capacity) of a heterologous functional agent (e.g., an agricultural or therapeutic agent). The NMPs or LNMPs may be modified in vitro or in vivo.

As used herein, the term “unmodified NMPs” or “unmodified LNMPs” refers to a composition including a plurality of NMPs or LNMPs that lack a heterologous cell uptake agent capable of increasing cell uptake (e.g., animal cell uptake, plant cell uptake, bacterial cell uptake, or fungal cell uptake) of the NMP.

As used herein, the term “modified PMPs” or “modified LPMPs” refers to a composition including a plurality of PMPs or LPMPs that include one or more heterologous agents (e.g., one or more exogenous lipids, such as a ionizable lipids, e.g., a PMP or LPMP comprising an ionizable lipid and a sterol and/or a PEGylated lipid) capable of increasing cell uptake (e.g., animal cell uptake, plant cell uptake, bacterial cell uptake, or fungal cell uptake) of the PMP or LPMP, or a portion or component thereof, relative to an unmodified PMP or LPMP; capable of enabling or increasing delivery of a heterologous functional agent (e.g., an agricultural or therapeutic agent) by the PMP or LPMP to a cell, and/or capable of enabling or increasing loading (e.g., loading efficiency or loading capacity) of a heterologous functional agent (e.g., an agricultural or therapeutic agent). The PMPs or LPMPs may be modified in vitro or in vivo.

As used herein, the term “unmodified PMPs” or “unmodified LPMPs” refers to a composition including a plurality of PMPs or LPMPs that lack a heterologous cell uptake agent capable of increasing cell uptake (e.g., animal cell uptake, plant cell uptake, bacterial cell uptake, or fungal cell uptake) of the PMP.

As used herein, the term “cell uptake” refers to uptake of a NMP or LNMP or a portion or component thereof (e.g., a polynucleotide carried by the NMP or LNMP) by a cell, such as an animal cell, a plant cell, bacterial cell, or fungal cell. For example, uptake can involve transfer of the NMP (e.g., LNMP) or a portion of component thereof from the extracellular environment into or across the cell membrane, the cell wall, the extracellular matrix, or into the intracellular environment of the cell). Cell uptake of NMPs (e.g., LNMPs) may occur via active or passive cellular mechanisms. Cell uptake includes aspects in which the entire NMP (e.g., LNMP) is taken up by a cell, e.g., taken up by endocytosis. In some embodiments, one or more polynucleotides are exposed to the cytoplasm of the target cell following endocytosis and endosomal escape. In some embodiments, a modified LNMP (e.g., a LNMP comprising an ionizable lipid, e.g., a LNMP comprising an ionizable lipid and a sterol and/or a PEGylated lipid) has an increased rate of endosomal escape relative to an unmodified LNMP. Cell uptake also includes aspects in which the NMP (e.g., LNMP) fuses with the membrane of the target cell. In some embodiments, one or more polynucleotides are exposed to the cytoplasm of the target cell following membrane fusion. In some embodiments, a LNMPs has an increased rate of fusion with the membrane of the target cell (e.g., is more fusogenic) relative to an unmodified LNMP.

As used herein, the term “cell-penetrating agent” refers to agents that alter properties (e.g., permeability) of the cell wall, extracellular matrix, or cell membrane of a cell (e.g., an animal cell, a plant cell, a bacterial cell, or a fungal cell) in a manner that promotes increased cell uptake relative to a cell that has not been contacted with the agent.

As used herein, the term “plant” refers to whole plants, plant organs, plant tissues, seeds, plant cells, seeds, and progeny of the same. Plant cells include, without limitation, cells from seeds, suspension cultures, embryos, meristematic regions, callus tissue, leaves, roots, shoots, gametophytes, sporophytes, pollen, and microspores. Plant parts include differentiated and undifferentiated tissues including, but not limited to the following: roots, stems, shoots, leaves, pollen, seeds, fruit, harvested produce, tumor tissue, sap (e.g., xylem sap and phloem sap), and various forms of cells and culture (e.g., single cells, protoplasts, embryos, and callus tissue). The plant tissue may be in a plant or in a plant organ, tissue, or cell culture. In addition, a plant may be genetically engineered to produce a heterologous protein or RNA.

As used herein, the term “Bacteria” refers to whole bacteria or parts of bacteria. Further divisions of bacteria can be classified as coccals, bacillus, spirillum, or vibrio, and varying phylums include but are not limited to Proteobacteria, Firmicutes, Bacteroids, sphingobacteria, Flavobacteria, Fusobacteria, Spirochaetes, Chlorobia, Cyanobacteria, Thermomicrobia, Xenobacteria, or Aquificae. Examples of specific bacteria species include Staphylococcus aureus, Escherichia coli, Salmonella typhimurium, Streptococcus pneumoniae, and Pseudomonas aeruginosa. Parts of bacteria include cellular components such as peptidoglycan, outer membranes, inner membranes, cell walls, RNA polymerase, metabolic products, polypeptides, proteins. Flagella, pili, ribosomes, mesosome, cytoplasm, or chromosome. Bacteria may be genetically engineered to produce a heterologous protein or RNA, or may be genetically engineered to not produce an endogenous protein or RNA.

As used herein, the term “Arthropod” refers to any animal within the phylum Arthropoda, or any animal section, part, organ, tissue, egg, cell, or progeny of the same. Example animals include insects, spiders, and crustaceans. Arthropod cells include, without limitation, cells from eggs, suspension cultures, embryos, tissue, organs, exoskeleton, segments, and appendages. Arthropod parts include body segments, appendages, exoskeleton, eggs, organs, embryos, and various forms of cells and culture. Arthropod tissue may be in an arthropod or in an organ, tissue, or cell culture. An arthropod may be genetically engineered to produce a heterologous protein or RNA. An arthropod may be genetically engineered to not produce an endogenous protein or RNA.

As used herein, the term “Fungi” refers to whole fungi, fungi organs, fungi tissue, spores, fungi cells, and progeny of the same. Example fungi include yeasts, mushrooms, molds, and mildews. Fungi cells include without limitation cells from spores, suspension cultures, mycelium, hyphae, thallus, cell walls, tissue, gametophytes, sporophytes, and organs. Fungal tissue may be in a fungus or in an organ, tissue, or cell culture. A fungus may be genetically engineered to produce a heterologous protein or RNA. A fungus may be genetically engineered to not produce an endogenous protein or RNA.

As used herein, the term “Archaea” refers to whole archaea or parts of archaea. Example archaea include euryarchaeota, crenarchaeota, and koraarchaeota. Parts of archaea include cellular components such as RNA polymerases, glycerol-ether lipids, membranes, cell walls, polypeptides, proteins, and metabolic products. An archaea may be genetically engineered to produce a heterologous protein or RNA, or may be genetically engineered to not produce an endogenous protein or RNA.

As used herein, the term “extracellular vesicle” or “EV” refers to an enclosed lipid-bilayer structure naturally occurring in an organism or cell. Optionally, the EV includes one or more EV markers. As used herein, the term “EV marker” refers to a component that is naturally associated with a specific organism, such as a protein, a nucleic acid, a small molecule, a lipid, or a combination thereof. In some instances, the EV marker is an identifying marker of an EV but is not a pesticidal agent. In some instances, the EV marker is an identifying marker of an EV and also a pesticidal agent (e.g., either associated with or encapsulated by the plurality of NMPs, or not directly associated with or encapsulated by the plurality of NMPs).

As used herein, the term “plant extracellular vesicle”, “plant EV”, or “EV” refers to an enclosed lipid-bilayer structure naturally occurring in a plant. Optionally, the plant EV includes one or more plant EV markers. As used herein, the term “plant EV marker” refers to a component that is naturally associated with a plant, such as a plant protein, a plant nucleic acid, a plant small molecule, a plant lipid, or a combination thereof, including but not limited to any of the plant EV markers listed in the Appendix. In some instances, the plant EV marker is an identifying marker of a plant EV but is not a pesticidal agent. In some instances, the plant EV marker is an identifying marker of a plant EV and also a pesticidal agent (e.g., either associated with or encapsulated by the plurality of PMPs or LPMPs, or not directly associated with or encapsulated by the plurality of PMPs or LPMPs).

As used herein, the term “natural messenger pack” or “NMP” refers to a lipid structure (e.g., a lipid bilayer, unilamellar, multilamellar structure; e.g., a vesicular lipid structure), that is about 5-2000 nm (e.g., at least 5-1000 nm, at least 5-500 nm, at least 400-500 nm, at least 25-250 nm, at least 50-150 nm, or at least 70-120 nm) in diameter that is derived from (e.g., enriched, isolated or purified from) a natural source or segment, portion, or extract thereof, including lipid or non-lipid components (e.g., peptides, nucleic acids, or small molecules) associated therewith and that has been enriched, isolated or purified from a natural source, a part of a natural source, or a cell of a natural source, the enrichment or isolation removing one or more contaminants or undesired components from the source. NMPs may be highly purified preparations of naturally occurring EVs. Preferably, at least 1% of contaminants or undesired components from the natural source are removed (e.g., at least 2%, 5%, 10%, 15%, 20%, 25%, 30%, 40%, 45%, 50%, 55%, 60%, 70%, 80%, 90%, 95%, 96%, 98%, 99%, or 100%) of one or more contaminants or undesired components from the source, e.g., source cell wall components; pectin; organelles (e.g., mitochondria; plastids such as chloroplasts, leucoplasts or amyloplasts; and nuclei); chromatin (e.g., a chromosome); or molecular aggregates (e.g., protein aggregates, protein-nucleic acid aggregates, lipoprotein aggregates, or lipido-proteic structures). Preferably, a NMP is at least 30% pure (e.g., at least 40% pure, at least 50% pure, at least 60% pure, at least 70% pure, at least 80% pure, at least 90% pure, at least 99% pure, or 100% pure) relative to the one or more contaminants or undesired components from the natural source as measured by weight (w/w), spectral imaging (% transmittance), or conductivity (S/m).

As used herein, the term “plant messenger pack” or “PMP” refers to a lipid structure (e.g., a lipid bilayer, unilamellar, multilamellar structure; e.g., a vesicular lipid structure), that is about 5-2000 nm (e.g., at least 5-1000 nm, at least 5-500 nm, at least 400-500 nm, at least 25-250 nm, at least 50-150 nm, or at least 70-120 nm) in diameter that is derived from (e.g., enriched, isolated or purified from) a plant source or segment, portion, or extract thereof, including lipid or non-lipid components (e.g., peptides, nucleic acids, or small molecules) associated therewith and that has been enriched, isolated or purified from a plant, a plant part, or a plant cell, the enrichment or isolation removing one or more contaminants or undesired components from the source plant. PMPs may be highly purified preparations of naturally occurring EVs. Preferably, at least 1% of contaminants or undesired components from the source plant are removed (e.g., at least 2%, 5%, 10%, 15%, 20%, 25%, 30%, 40%, 45%, 50%, 55%, 60%, 70%, 80%, 90%, 95%, 96%, 98%, 99%, or 100%) of one or more contaminants or undesired components from the source plant, e.g., plant cell wall components; pectin; plant organelles (e.g., mitochondria; plastids such as chloroplasts, leucoplasts or amyloplasts; and nuclei); plant chromatin (e.g., a plant chromosome); or plant molecular aggregates (e.g., protein aggregates, protein-nucleic acid aggregates, lipoprotein aggregates, or lipido-proteic structures). Preferably, a PMP is at least 30% pure (e.g., at least 40% pure, at least 50% pure, at least 60% pure, at least 70% pure, at least 80% pure, at least 90% pure, at least 99% pure, or 100% pure) relative to the one or more contaminants or undesired components from the source plant as measured by weight (w/w), spectral imaging (% transmittance), or conductivity (S/m).

A lipid reconstructed NMP (LNMP) is used herein. For instance, a lipid reconstructed PMP (LPMP) is used herein. The terms “lipid reconstructed NMP” and “LNMP” refer to a NMP that has been derived from a lipid structure (e.g., a lipid bilayer, unilamellar, multilamellar structure; e.g., a vesicular lipid structure) derived from (e.g., enriched, isolated or purified from) a natural source, wherein the lipid structure is disrupted (e.g., disrupted by lipid extraction) and reassembled or reconstituted in a liquid phase (e.g., a liquid phase containing a cargo) using standard methods, e.g., reconstituted by a method comprising lipid film hydration and/or solvent injection, to produce the LNMP, as is described herein. The method may, if desired, further comprise sonication, freeze/thaw treatment, and/or lipid extrusion, e.g., to reduce the size of the reconstituted NMPs. Alternatively, LNMPs may be produced using a microfluidic device (such as a NanoAssemblr® IGNITE™ microfluidic instrument (Precision NanoSystems)). The terms “lipid reconstructed PMP” and “LPMP” are defined in the same manner as “lipid reconstructed NMP” and “LNMP,” when the natural source is a plant source.

As used herein, the term “pure” refers to a PMP preparation in which at least a portion (e.g., at least 20%, 25%, 30%, 40%, 45%, 50%, 55%, 60%, 70%, 80%, 90%, 95%, 96%, 98%, 99%, or 100%) of plant cell wall components, plant organelles (e.g., mitochondria, chloroplasts, and nuclei), or plant molecule aggregates (protein aggregates, protein-nucleic acid aggregates, lipoprotein aggregates, or lipido-proteic structures) have been removed relative to the initial sample isolated from a plant, or part thereof.

As used herein, the term “complex lipid particle” refers to a lipid particle that has a complexity characterized by comprising a wide variety of lipids, including structural lipids extracted from one or more natural sources (such as plants or bacteria), and optionally at least one exogenous ionizable lipid. The complex lipid particle may comprise between 10% w/w and 99% w/w structural lipids derived from a lipid structure from one or more natural sources, e.g., it may contain at least 10% w/w, at least 20% w/w, at least 30% w/w, at least 40% w/w, at least 50% w/w, at least 60% w/w, at least 70% w/w, at least 80% w/w, at least 90% w/w, at least 95% w/w, or about 99% w/w lipids derived from a lipid structure from one or more natural sources. In some instances, a complex lipid particle incorporating natural lipid extracts may also be referred to as a natural messenger pack (NMP). For instance, a complex lipid particle incorporating plant lipid extracts may also be referred to as a plant messenger pack (PMP). In some instances, a complex lipid particle incorporating natural lipid extracts and at least one exogenous ionizable lipid may also be referred to as a lipid reconstructed natural messenger pack (LNMP). For instance, a complex lipid particle incorporating plant lipid extracts and at least one exogenous ionizable lipid may also be referred to as a lipid reconstructed plant messenger pack (LPMP). Thus, any disclosure herein describing the features relating to LNMP and LNMP formulation are applicable to CLP and CLP formulation.

The complex lipid particle may contain 3-1000 lipids extracted from one or more natural (e.g., plant, bacteria) sources. The complex lipid particle may contain natural (e.g., plant, bacteria) lipids from at least 1, at least 2, at least 3, at least 4, at least 5, at least 6, at least 7, at least 8, at least 9, or at least 10 different classes or sub-classes of lipids from the natural (e.g., plant, bacteria) source. The complex lipid particle may comprise all or a fraction of the lipid species present in the lipid structure from the natural (e.g., plant, bacteria) source, e.g., it may contain at least 10%, at least 20%, at least 30%, at least 40%, at least 50%, at least 60%, at least 70%, at least 80%, at least 90%, or virtually 100% of the lipid species present in the lipid structure from the natural source. The complex lipid particle may comprise all or a fraction of the lipid species present in the lipid structure from a particular natural source. For instance, it may contain at least 10%, at least 20%, at least 30%, at least 40%, at least 50%, at least 60%, at least 70%, at least 80%, at least 90%, or virtually 100% of the lipid species present in the lipid structure from a plant source or from a bacteria source.

The complex lipid particle may comprise reduced or minimized protein matter endogenous to the one or more natural (i.e. plant, bacteria) sources, e.g., it may contain 0% w/w, less than 1% w/w, less than 5% w/w, less than 10% w/w, less than 15% w/w, less than 20% w/w, less than 30% w/w, less than 40% w/w, or less than 50% w/w of the protein matter endogenous to the one or more natural (e.g., plant, bacteria) sources. In some instances, the lipid bilayer of the complex lipid particle does not contain proteins.

The complex lipid particle may also include synthetic structural lipids such as neutral lipids as the structural lipid component. The structural lipid component of the complex lipid particle may comprise between 10% w/w and 99% w/w structural lipids derived from a synthetic lipid structure (as opposed to the lipids extracted from a natural source), e.g., it may contain at least 10% w/w, at least 20% w/w, at least 30% w/w, at least 40% w/w, at least 50% w/w, at least 60% w/w, at least 70% w/w, at least 80% w/w, at least 90% w/w, at least 95% w/w, or about 99% w/w lipids derived from a synthetic lipid structure.

The complex lipid particle may further comprise at least two exogenous lipids. The complex lipid particle may include at least 1% w/w, at least 2% w/w, at least 5% w/w, at least 10% w/w, at least 15% w/w, at least 20% w/w, at least 25% w/w, at least 30% w/w, at least 40% w/w, at least 50% w/w, at least 60% w/w, at least 70% w/w, at least 80% w/w, or about 90% w/w exogenous lipids. Exemplary exogenous lipids include sterols and PEG-lipid conjugate. The complex lipid particle may be used to encapsulate one or more exogenous nucleic acids or polynucleotides encoding one or more peptides, polypeptides, or proteins, to enable delivery of the exogenous nucleic acids or polynucleotides to a target cell or tissue.

As used herein, the term “exogenous lipid” refers to a lipid that is exogenous to the natural source (e.g., plant, bacteria), i.e., a lipid originates from a source that is not the natural source from which the lipids are extracted (e.g., a lipid that is added to the complex lipid particle formulation using method described herein). The term “exogenous lipid” does not exclude a natural-derived lipid (such as a plant-derived sterol). That is to say, an exogenous lipid can be a natural-derived lipid (such as a plant-derived sterol that is exogenous to the plant source from which the lipids are extracted, e.g., an exogenous lipid can be a plant derived sterol that is added to the complex lipid particle formulation). As another example, an exogenous lipid can be a natural-derived lipid that is exogenous to the particular natural source from which the lipids are extracted (e.g., a bacteria-derived lipid that is exogenous to the plant source from which the lipids are extracted, or vice versa). An exogenous lipid may be a cell-penetrating agent, may be capable of increasing delivery of one or more polynucleotides by the complex lipid formulation to a cell, and/or may be capable of increasing loading (e.g., loading efficiency or loading capacity) of a polynucleotide. In some embodiments, the exogenous lipid may be a stabilizing lipid. In some embodiments, the exogenous lipid may be a structural lipid (e.g., a synthetic structural lipid). Exemplary exogenous lipids include ionizable lipids, synthetic structural lipids, sterols, and PEGylated lipids.

As used herein, the term “cationic lipid” refers to an amphiphilic molecule (e.g., a lipid or a lipidoid) that is positively charged, containing a cationic group (e.g., a cationic head group).

As used herein, the term “ionizable lipid” refers to an amphiphilic molecule (e.g., a lipid or a lipidoid, e.g., a synthetic lipid or lipidoid) containing a group (e.g., a head group) that can be ionized, e.g., dissociated to produce one or more electrically charged species, under a given condition (e.g., pH).

It has been surprisingly found that ionizable lipids comprising alkyl chains with multiple sites of unsaturation, e.g., at least two or three sites of unsaturation are particularly useful for forming lipid particles with increased membrane fluidity. A number of ionizable lipids and related analogs, suitable for use herein, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes.

In some embodiments, ionizable lipids are ionizable such that they can dissociate to exist in a positively charged form depending on pH. The ionization of an ionizable lipid affects the surface charge of a lipid nanoparticle comprising the ionizable lipid under different pH conditions. The surface charge of the lipid nanoparticle in turn can influence its plasma protein absorption, blood clearance, and tissue distribution (Semple, S. C., et al., Adv. Drug Deliv Rev 32:3-17 (1998)) as well as its ability to form endosomolytic non-bilayer structures (Hafez, I. M., et al., Gene Ther 8: 1188-1196 (2001)) that can influence the intracellular delivery of nucleic acids.

In some embodiments, ionizable lipids are those that are generally neutral, e.g., at physiological pH (e.g., pH about 7), but can carry net charge(s) at an acidic pH or basic pH. In one embodiment, ionizable lipids are those that are generally neutral at pH about 7, but can carry net charge(s) at an acidic pH. In one embodiment, ionizable lipids are those that are generally neutral at pH about 7, but can carry net charge(s) at a basic pH.

In some embodiments, ionizable lipids do not include those cationic lipids or anionic lipids that generally carry net charge(s) at physiological pH (e.g., pH about 7).

As used herein, the term “lipidoid” refers to a molecule having one or more characteristics of a lipid.

As used herein, the term “stable LNMP formulation” or “stable CLP formulation” refers to a CLP formulation or a LNMP composition that over a period of time (e.g., at least 24 hours, at least 48 hours, at least 1 week, at least 2 weeks, at least 3 weeks, at least 4 weeks, at least 30 days, at least 60 days, or at least 90 days) retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of the initial number of CLPs or LNMPs (e.g., CLPs or LNMPs per mL of solution) relative to the number of CLPs or LNMPs in the CLP formulation or LNMP formulation (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)); or retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of its activity (e.g., cell wall penetrating activity and/or activity of the RNA formulated within the CLP or LNMP) relative to the initial activity of the CLP or LNMP (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

Alternatively, the expression refers to a CLP formulation or LNMP composition that over a period of time (e.g., at least 24 hours, at least 48 hours, at least 1 week, at least 2 weeks, at least 3 weeks, at least 4 weeks, at least 30 days, at least 60 days, or at least 90 days) retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of its activity relative to the initial activity of the CLP formulation or LNMP formulation (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

Alternatively, the expression refers to a CLP formulation or a LNMP formulation that over a period of time (e.g., at least 24 hours, at least 48 hours, at least 1 week, at least 2 weeks, at least 3 weeks, at least 4 weeks, at least 30 days, at least 60 days, or at least 90 days) retains their particle size, i.e., the particle size does not increase, or has an increase of no more than 5% (e.g., no more than 5%, 10%, 15%, 20%, 25%, 30%, 35%, 40%, 45%, 50%, 55%, 60%, 65%, 70%, 75%, 80%, 85%, 90%, 95%, 100%, 150%, 2-fold, 2.5-fold, or 3-fold) relative to the initial size of the CLPs or LNMPs (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

In some embodiments, the stable CLP or LNMP formulation continues to encapsulate or remains associated with an exogenous peptide, polypeptide, or protein with which the CLP or LNMP formulation has been loaded, e.g., continues to encapsulate or remains associated with an exogenous peptide, polypeptide, or protein for at least 24 hours, at least 48 hours, at least 1 week, at least 2 weeks, at least 3 weeks, at least 4 weeks, at least 30 days, at least 60 days, at least 90 days, or 90 or more days.

As used herein, the term “treatment” refers to administering a pharmaceutical composition to an animal for prophylactic and/or therapeutic purposes. To “prevent an infection” refers to prophylactic treatment of an animal that does not yet have a disease or condition, but which is susceptible to, or otherwise at risk of, a particular disease or condition. To “treat an infection” refers to administering treatment to an animal already suffering from a disease to improve or stabilize the animal's condition.

As used herein, the term “treat an infection” refers to administering treatment to an individual (e.g., an animal) already having a disease to improve or stabilize the individual's condition. This may involve reducing colonization of a pathogen in, on, or around an animal by one or more pathogens (e.g., by about 1%, 2%, 5%, 10%, 20%, 30%, 40%, 50%, 60%, 70%, 80%, 90%, or 100%) relative to a starting amount and/or allow benefit to the individual (e.g., reducing colonization in an amount sufficient to resolve symptoms). In such instances, a treated infection may manifest as a decrease in symptoms (e.g., by about 1%, 2%, 5%, 10%, 20%, 30%, 40%, 50%, 60%, 70%, 80%, 90%, or 100%). In some instances, a treated infection is effective to increase the likelihood of survival of an individual (e.g., an increase in likelihood of survival by about 1%, 2%, 5%, 10%, 20%, 30%, 40%, 50%, 60%, 70%, 80%, 90%, or 100%) or increase the overall survival of a population (e.g., an increase in likelihood of survival by about 1%, 2%, 5%, 10%, 20%, 30%, 40%, 50%, 60%, 70%, 80%, 90%, or 100%). For example, the compositions and methods may be effective to “substantially eliminate” an infection, which refers to a decrease in the infection in an amount sufficient to sustainably resolve symptoms (e.g., for at least 1, 2, 3, 4, 5, 6, 7, 8, 9, 10, 11, or 12 months) in the animal.

As used herein, the term “prevent an infection” refers to preventing an increase in colonization in, on, or around an animal by one or more pathogens (e.g., by about 1%, 2%, 5%, 10%, 20%, 30%, 40%, 50%, 60%, 70%, 80%, 90%, 100%, or more than 100% relative to an untreated animal) in an amount sufficient to maintain an initial pathogen population (e.g., approximately the amount found in a healthy individual), prevent the onset of an infection, and/or prevent symptoms or conditions associated with infection. For example, an individual (e.g., an animal, e.g., a human) may receive prophylaxis treatment to prevent a fungal infection while being prepared for an invasive medical procedure (e.g., preparing for surgery, such as receiving a transplant, stem cell therapy, a graft, a prosthesis, receiving long-term or frequent intravenous catheterization, or receiving treatment in an intensive care unit), in immunocompromised individuals (e.g., individuals with cancer, with HIV/AIDS, or taking immunosuppressive agents), or in individuals undergoing long term antibiotic therapy.

### BRIEF DESCRIPTION OF THE DRAWINGS

FIG. 1A shows the levels of antibody (IgG) specific to the receptor binding domain (RBD) of SARS-CoV-2 in the blood of mice after 14 days, after a single dose intramuscular delivery of the various LNMP formulations employing the novel ionizable lipids disclosed herein (S mRNA 10 μg, see the LNMP formulations in Table 2). N=5/group. Controls were PBS and C12-200 LNP. N=5/group. FIG. 1B shows the number of SARS-CoV-2 S-specific T cells producing cytokine IFNγ per 10⁶ splenocytes in mice at 14 days, after a single dose intramuscular delivery of the various LNMP formulations employing the novel ionizable lipids disclosed herein (S mRNA 10 μg, see the LNMP formulations in Table 2). N=5/group. Controls were PBS and C12-200 LNP. N=5/group.

FIG. 2A shows the levels of antibody (IgG) specific to the receptor binding domain (RBD) of SARS-CoV-2 in the blood of mice after 14 days, after a single dose intramuscular delivery of the various LNMP formulations employing the novel ionizable lipids disclosed herein (S mRNA 10 μg, see the LNMP formulations in Table 2). N=5/group. Controls were PBS and C12-200 LNP. N=5/group. FIG. 2B shows the number of SARS-CoV-2 S-specific T cells producing cytokine IFNγ per 10⁶ splenocytes in mice at 14 days, after a single dose intramuscular delivery of the various LNMP formulations employing the novel ionizable lipids disclosed herein (S mRNA 10 μg, see the LNMP formulations in Table 2). N=5/group. Controls were PBS and C12-200 LNP. N=5/group.

FIGS. 3A and 3B show the levels of S-specific IgA in the blood of hamsters after one or two intramuscular doses of various LNMP formulations (S mRNA 20 μg, see the LNMP formulations in Table 4) on D23 (after one dose, FIG. 3A) and D42 (after two doses, FIG. 3B), respectively. N=12/group. Controls were the buffer solution and a commercial bivalent vaccine. N=12/group.

FIGS. 4A and 4B show the levels of RBD-specific IgG in the blood of hamsters after one or two intramuscular doses of various LNMP formulations (S mRNA 20 μg, see the LNMP formulations in Table 4) on D23 (after one dose, FIG. 4A) and D42 (after two doses, FIG. 4B), respectively. N=12/group. Controls were the buffer solution and a commercial bivalent vaccine. N=12/group.

FIG. 5A shows the percentage body weight change as mean across all groups of hamsters after two intramuscular doses of various LNMP formulations (S mRNA 20 μg, see the LNMP formulations in Table 4), plotted against days after a SARS-CoV-2 challenge. Controls were the buffer solution and a commercial bivalent vaccine. FIG. 5B shows the infectious viral titers (TCID50) in nares across all groups of hamsters after two intramuscular doses of various LNMP formulations (S mRNA 20 μg, see the LNMP formulations in Table 4) and 4 days after a SARS-CoV-2 challenge. Controls were the buffer solution and a commercial bivalent vaccine. N=6/group. FIG. 5C shows the infectious viral titers (TCID50) in lungs across all groups of hamsters after two intramuscular doses of various LNMP formulations (S mRNA 20 μg, see the LNMP formulations in Table 4) and 4 days after a SARS-CoV-2 challenge. Controls were the buffer solution and a commercial bivalent vaccine. N=6/group. Measured values that produced less TCID50 than the lower limit of detection (LLOD) were assigned the LLOD value.

FIG. 6A shows the levels of neutralizing antibody titer against the SARS-CoV-2 original strain in the blood of the hamsters at 23 days, after a single intramuscular vaccination of the various LNMP formulations (S mRNA 20 μg, see the LNMP formulations in Table 4). N=12/group. Controls were a buffer solution and a commercial bivalent vaccine. N=12/group. FIG. 6B shows the levels of neutralizing antibody titer against the SARS-CoV-2 original strain in the blood of the hamsters at 42 days, after two doses of intramuscular vaccination of the various LNMP formulations (S mRNA 20 μg, see the LNMP formulations in Table 4). N=12/group. Controls were a buffer solution and a commercial bivalent vaccine. N=12/group. FIG. 6C shows the levels of neutralizing antibody titer against the SARS-CoV-2 original strain in the blood of the hamsters at 50 days, after two doses of intramuscular vaccination of the various LNMP formulations (S mRNA 20 μg, see the LNMP formulations in Table 4) and 7 days post-challenge. N=12/group. Controls were a buffer solution and a commercial bivalent vaccine. N=12/group.

FIG. 7A shows the level of S-specific IgG in the blood of hamsters after one intramuscular dose of 2425 LPMP 2/SARS-CoV-2 (see soy LPMP in Table 4, containing S mRNA, 10 ug) or 2425 LPMP 2/SARS-CoV-2 (see soy LPMP in Table 4, containing S mRNA, 1 ug) on Day 21 post-dose. N=14/group. Controls were the monovalent vaccine (1 ug, n=14) and pooled naïve hamsters. FIG. 7B shows the level of RBD-specific IgG in the blood of hamsters after one intramuscular dose of 2425 LPMP 2/SARS-CoV-2 (see soy LPMP in Table 4, containing S mRNA, 10 ug) or 2425 LPMP 2/SARS-CoV-2 (see soy LPMP in Table 4, containing S mRNA, 1 ug) on Day 21 post-dose. N=14/group. Controls were the monovalent vaccine (1 ug, n=14) and pooled naïve hamsters.

FIG. 8A shows the amount of S-specific IgG in the blood of hamsters after one intramuscular dose of 2425 LPMP 2/SARS-CoV-2 (see soy LPMP in Table 4, containing S mRNA, 10 ug) or 2425 LPMP 2/SARS-CoV-2 (see soy LPMP in Table 4, containing S mRNA, 1 ug) on Day 21 post-dose. N=14/group. Controls were the monovalent vaccine (1 ug, n=14) and pooled naïve hamsters. FIG. 8B shows the amount of RBD-specific IgG in the blood of hamsters after one intramuscular dose of 2425 LPMP 2/SARS-CoV-2 (see soy LPMP in Table 4, containing S mRNA, 10 ug) or 2425 LPMP 2/SARS-CoV-2 (see soy LPMP in Table 4, containing S mRNA, 1 ug) on Day 21 post-dose. N=14/group. Controls were the monovalent vaccine (1 ug, n=14) and pooled naïve hamsters.

FIG. 9 shows the percentage of inhibition in hamsters after one intramuscular dose of 2425 LPMP 2/SARS-CoV-2 (see soy LPMP in Table 4, containing S mRNA, 10 ug) or 2425 LPMP 2/SARS-CoV-2 (see soy LPMP in Table 4, containing S mRNA, 1 ug) on Day 21 post-dose. N=14/group. Controls were the monovalent vaccine (1 ug, n=14).

FIG. 10A shows the absolute number of germinal center B cells in pooled draining lymph nodes seven or ten days after one intramuscular dose of various LNMP formulations employing the novel ionizable lipids disclosed herein (S mRNA 10 μg/40 μL, see the LNMP formulations in Table 6). N=6/group. Controls were the LNP formulation (see Table 6) or naïve mice. FIG. 10B shows the absolute number of T follicular helper cells in pooled draining lymph nodes seven or ten days after one intramuscular dose of various LNMP formulations employing the novel ionizable lipids disclosed herein (S mRNA 10 μg/40 μL, see the LNMP formulations in Table 6). N=6/group. Controls were the LNP formulation (see Table 6) or naïve mice. FIG. 10C shows the frequency of germinal center B cells among B cells in pooled lymph nodes seven or ten days after one intramuscular dose of various LNMP formulations employing the novel ionizable lipids disclosed herein (S mRNA 10 μg/40 μL, see the LNMP formulations in Table 6). N=6/group. Controls were the LNP formulation (see Table 6) or naïve mice. FIG. 10D shows the frequency of T follicular helper cells among CD4 T cells in pooled lymph nodes seven or ten days after one intramuscular dose of various LNMP formulations employing the novel ionizable lipids disclosed herein (S mRNA 10 μg/40 μL, see the LNMP formulations in Table 6). N=6/group. Controls were the LNP formulation (see Table 6) or naïve mice. FIG. 10E shows the absolute number of class switched B cells in pooled lymph nodes seven or ten days after one intramuscular dose of various LNMP formulations employing the novel ionizable lipids disclosed herein (S mRNA 10 μg/40 μL, see the LNMP formulations in Table 6). N=6/group. Controls were the LNP formulation (see Table 6) or naïve mice.

FIG. 11A shows the whole body radiance (average radiance p/s/cm²/sr) at 4 hours post-dose of intramuscular administration of various exemplary LNMPs and LPMPs formulations employing the novel ionizable lipids disclosed herein (see Table 7) encapsulating 1:1 mRNA FLuc:hEPO, dosed at 10 ug/40 uL, in mice. N=3/group. Positive controls used were LNP 1 and LNP 2 (n=3/group), and negative control was mice dosed with PBS (n=2). FIGS. 11B and 11C show the liver (FIG. 11B) and spleen (FIG. 11C) radiance (average radiance p/s/cm²/sr) at 4 hours post-dose of intramuscular administration of various exemplary LNMPs and LPMPs formulations employing the novel ionizable lipids disclosed herein (see Table 7) encapsulating 1:1 mRNA Fluc:hEPO, dosed at 10 ug/40 uL, in mice. N=3/group. Positive controls used were LNP 1 and LNP 2 (n=3/group), and negative control was mice dosed with PBS (n=2). FIG. 11D shows the lung and quad muscle (injection site) radiance (average radiance p/s/cm²/sr) at 4 hours post-dose of intramuscular administration of various exemplary LNMPs and LPMPs formulations employing the novel ionizable lipids disclosed herein (see Table 7) encapsulating 1:1 mRNA Fluc:hEPO, dosed at 10 ug/40 uL, in mice. N=3/group. Positive controls used were LNP 1 and LNP 2 (n=3/group), and negative control was mice dosed with PBS (n=2). FIG. 11E shows the right and left side lymph nodes radiance (average radiance p/s/cm²/sr) at 4 hours post-dose of intramuscular administration of various exemplary LNMPs and LPMPs formulations employing the novel ionizable lipids disclosed herein (see Table 7) encapsulating 1:1 mRNA Fluc:hEPO, dosed at 10 ug/40 uL, in mice. N=3/group. Positive controls used were LNP 1 and LNP 2 (n=3/group), and negative control was mice dosed with PBS (n=2).

FIG. 12A shows the levels of RBD-specific IgG in the blood of mice at 21 days, after a single intramuscular vaccination of the LNMP formulations employing an exemplary novel ionizable lipid 2425 (S mRNA 5 μg, see the LNMP formulations in Table 8). N=5/group. FIG. 12B shows the levels of RBD-specific IgG in the blood of mice at 42 days, after two doses of intramuscular vaccination of the various LNMP formulations employing an exemplary novel ionizable lipid 2425 (S mRNA 5 μg, see the LNMP formulations in Table 8). N=5/group. FIG. 12C shows the number of SARS-CoV-2 S-specific T cells producing cytokine IFNγ per 10⁶ splenocytes in mice at 42 days, after two doses of intramuscular vaccination of the various LNMP formulations (S mRNA 5 μg, see the LNMP formulations in Table 8). N=5/group. FIG. 12D shows the levels of neutralizing antibody titer against the SARS-CoV-2 original strain in the blood of the mice at 42 days, after two doses of intramuscular vaccination of the various LNMP formulations (S mRNA 5 μg, see the LNMP formulations in Table 8). N=5/group. Controls in these figures were a buffer solution, LNP 1 containing Lipid 8 as ionizable lipid, 2425 LNP containing novel ionizable lipid 2425, and a commercial bivalent vaccine. N=5/group.

FIG. 13A shows the levels of RBD-specific IgG in the blood of mice at 21 days, after a single intramuscular vaccination of various LNMP formulations employing an exemplary novel ionizable lipid 2356 (S mRNA 5 μg, see the LNMP formulations in Table 10). N=5/group. FIG. 13B shows the levels of RBD-specific IgG in the blood of mice at 42 days, after two doses of intramuscular vaccination of various LNMP formulations employing an exemplary novel ionizable lipid 2356 (S mRNA 5 μg, see the LNMP formulations in Table 10). N=5/group. FIG. 13C shows the number of SARS-CoV-2 S-specific T cells producing cytokine IFNγ per 10⁶ splenocytes in mice at 42 days, after two doses of intramuscular vaccination of various LNMP formulations employing an exemplary novel ionizable lipid 2356 (S mRNA 5 μg, see the LNMP formulations in Table 10). N=5/group. FIG. 13D shows the levels of neutralizing antibody titer against the SARS-CoV-2 original strain in the blood of the mice at 42 days, after two doses of intramuscular vaccination of various LNMP formulations employing an exemplary novel ionizable lipid 2356 (S mRNA 5 μg, see the LNMP formulations in Table 10). N=5/group. Controls in these figures were a buffer solution, 2356 LNP containing novel ionizable lipid 2356, and a commercial bivalent vaccine. N=5/group.

FIG. 14A shows the levels of RBD-specific IgG in the blood of mice at 14 days, after a single intramuscular vaccination of various LNMP formulations employing an exemplary novel ionizable lipid 2425 (S mRNA 0.4 μg, 1 ug, or 10 ug, see the LNMP formulations in Table 12). N=5/group. FIG. 14B shows the levels of RBD-specific IgG in the blood of mice at 28 days, after a single dose of intramuscular vaccination of various LNMP formulations employing an exemplary novel ionizable lipid 2425 (S mRNA 0.4 μg, 1 ug, or 10 ug, see the LNMP formulations in Table 12). N=5/group. FIG. 14C shows the number of SARS-CoV-2 S-specific T cells producing cytokine IFNγ per 10⁶ splenocytes in mice at 28 days, after a single intramuscular vaccination of the various LNMP formulations employing an exemplary novel ionizable lipid 2425 (S mRNA 0.4 μg, 1 ug, or 10 ug, see the LNMP formulations in Table 12). N=5/group. Controls in these figures were a buffer solution, LNP 1 containing Lipid 8 as ionizable lipid, 2425 LNP containing novel ionizable lipid 2425, and a commercial bivalent vaccine. N=5/group.

### DETAILED DESCRIPTION

Featured herein are nucleic acid vaccine compositions (e.g., immunizing/immunogenic compositions), that elicit potent neutralizing antibodies against an antigen of an infectious disease, disorder, or condition (e.g., coronavirus antigens such as a SARS-CoV-2 antigen). These nucleic acid vaccine compositions include one or more polynucleotides (e.g., RNA such as mRNA or circRNA) encoding one or more antigenic polypeptides, formulated within a complex lipid particle (CLP). In some embodiments, the CLP is a lipid reconstructed natural messenger packs (LNMPs) comprising lipids extracted from one or more natural sources (i.e., natural lipids) and an ionizable lipid. The antigenic polypeptides are derived from an infectious agent that causes an infectious disease, disorder, or condition. NMPs are lipid assemblies produced wholly or in part from natural extracellular vesicles (EVs), or segments, portions, or extracts thereof. PMPs are lipid assemblies produced wholly or in part from plant extracellular vesicles (EVs), or segments, portions, or extracts thereof. LNMPs are NMPs derived from a lipid structure wherein the lipid structure is disrupted and reassembled or reconstituted in a liquid phase. LPMPs are PMPs derived from a lipid structure wherein the lipid structure is disrupted and reassembled or reconstituted in a liquid phase.

The disclosure also includes a method for making a nucleic acid vaccine, comprising reconstituting a film comprising purified NMP lipids in the presence of an ionizable lipid to produce a LNMP comprising the ionizable lipid, and loading into the LNMPs with one or more polynucleotides encoding one or more antigenic polypeptide-modified natural messenger packs (NMPs).

### Complex Lipid Particles and Lipid Reconstructed Natural Messenger Packs (LNMPs)

### Complex Lipid Particles

Complex lipid particles (CLPs) described herein comprise a wide variety of lipids, including structural lipids extracted from one or more natural sources (such as plants or bacteria). In some embodiments, a complex lipid particle is a natural messenger pack (NMP) incorporating natural lipid extracts. In some embodiments, a complex lipid particle is a lipid reconstructed natural messenger pack (LNMP) incorporating natural lipid extracts and at least one exogenous ionizable lipid.

The complex lipid particles may also comprise at least exogenous ionizable lipid. The ionizable lipid has two or more of the characteristics listed below:
(i) at least 2 ionizable amines;
(ii) at least 3 lipid tails; wherein each of the lipid tails is at least 6 carbon atoms in length;
(iii) a pKa of about 4.5 to about 7.5;
(iv) an ionizable amine and a heterorganic group separated by a chain of at least two atoms; and
(v) an N:P ratio of at least 3.

The complex lipid particle may comprise between 10% w/w and 99% w/w structural lipids derived from a lipid structure from one or more natural sources, e.g., it may contain at least 10% w/w, at least 20% w/w, at least 30% w/w, at least 40% w/w, at least 50% w/w, at least 60% w/w, at least 70% w/w, at least 80% w/w, at least 90% w/w, at least 95% w/w, or about 99% w/w lipids derived from a lipid structure from one or more natural sources.

In some embodiments, the complex lipid particle comprises about 10-95% w/w of the natural (e.g., plant, bacteria) lipids. For instance, the complex lipid particle comprises about 25-95% w/w, about 30-95% w/w, about 35-95% w/w, about 40-95% w/w, about 45-95% w/w, about 50-95% w/w, about 55-95% w/w, about 60-95% w/w, about 65-95% w/w, about 70-95% w/w, about 75-95% w/w, about 80-95% w/w, or about 85-95% w/w of the natural (e.g., plant, bacteria) lipids based on the amounts of total lipids in the complex lipid formulation.

The complex lipid particle may contain 3-1000 lipids extracted from one or more natural (e.g., plant, bacteria) sources. In some embodiments, the natural source is a plant, plant extract, or fragment or part of a plant. In some embodiments, the natural source is a bacteria, bacteria fragment or part of a bacteria. In some embodiments, the natural source is lemon. In some embodiments the natural source is soy. In other embodiments, the natural source is E. coli.

In some embodiments, the complex lipid particle contains at least 10 natural lipids belonging to one or more of the classes selected from the group consisting of fatty acyls (FA), fatty acyl conjugates, phospholipids, glycerolipids, glycolipids, glycerophospholipids, sphingolipids, waxes, and sterol. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, or at least 800 natural lipids belonging to one or more of the classes selected from the group consisting of fatty acyls (FA), fatty acyl conjugates, phospholipids, glycerolipids, glycolipids, glycerophospholipids, sphingolipids, waxes, and sterol. In some embodiments, the complex lipid particle contains lipids from at least two or at least three of these different classes.

In some embodiments, the complex lipid particle contains at least 10 natural lipids belonging to one or more of the classes selected from the group consisting of glycerolipid, sphingolipid, and sterol. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, or at least 800 natural lipids belonging to one or more of the classes selected from the group consisting of glycerolipid, sphingolipid, and sterol. In some embodiments, the complex lipid particle contains lipids from at least two or at least three of these different classes.

In some embodiments, the complex lipid particle may contain one or more glycerolipids (GL) or glycerophospholipids (GP), which may also include glycolipids.

In some embodiments, the complex lipid particle may contain one or more glycerolipids selected from the group consisting of phospholipids (PL), galactolipids, triacylglycerols (TG), and sulfolipids (SL). In some embodiments, the CLPs contains one or more glycerophospholipids (GP) selected from the group consisting of phosphatidylcholines (PC), phosphatidylethanolamines (PE), phosphatidylserines (PS), and phosphatidylinositols (PI). In some embodiments, the complex lipid particle contains one or more sphingolipids (SP) selected from the group consisting of sulfolipids (SL), glycosyl inositol phosphoryl ceramides (GIPC), glucosylceramides (GCer), ceramides (Cer), and free long-chain bases (LCB). In some embodiments, the complex lipid particle contains one or more phytosterols selected from the group consisting of campesterol, stigmasterol, β-sitosterol, Δ5-avenasterol, brassicasterol, avenasterol, 4-desmethyl sterol, 4α-monomethyl sterol, Δ5-sterol, Δ7-sterol, α-spinasterol, Δ5,Δ7-sterol, phytostanol, and sitosterol.

The CLP may contain one or more natural lipids belonging to one or more classes or sub-classes selected from the group consisting of fatty acids, fatty esters, fatty aldehydes, fatty amides, acyclic oxylipins, cyclic oxylipins, glycerolipids, monoradylglycerols, diradylglycerols, triradylglycerols, estolides, glycosylmonoacylglycerols, sulfoquinovosylmonoacylglycerols, monogalactosylmonoacylglycerol, digalactosylmonoacylglycerol, sulfoquinovosyldiacylglycerols, monogalactosyldiacylglycerol, digalactosyldiacylglycerol, glycosyldiacylglycerols, glyceropphospholipids, phospholipids, lysophospholipids, phosphatidylinositol phosphates, n-modified phospholipids, oxygenated/oxidized phospholipids, shingolipids, sphingoid bases, ceramides, phosphocereamides, glycophingolipids, sterols, cholesterol, cholesteryl ester, steryl esters, bile acids, sterylglycosides, and acylsterylglycosides. The complex lipid particle may contain one or more natural lipids belonging to one or more of the classes or sub-classes selected from the group consisting of the classes or sub-classes listed above. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, or at least 800 natural lipids belonging to one or more of the classes or sub-classes selected from the group consisting of the classes or sub-classes listed above.

In some embodiments, the CLP contains one or more natural lipids belonging to one or more of the sub-classes selected from the group consisting of acyl diacylglyceryl glucuronides, acylhexosylceramides, acylsterylglycosides, bile acids, acyl carnitines, cholesteryl esters, ceramides, cardiolipins, coenzyme Qs, diacylglycerols, digalactosyldiacylglycerols, diacylglyceryl glucuronides, dilysocardiolipins, fatty acids, fatty acid esters of hydroxyl fatty acids, hemibismonoacylglycerophosphates, hexosylceramides, lysophosphatidic acids, lysophophatidylcholines, lysophosphatidylethanolamines, N-acyl-lysophosphatidylethanolamines, lysophosphatidylglycerols, lysophosphatidylinositols, lysophosphatidylserines, monogalactosyldiacylglycerols, lysocardiolipins, N-acyl ethanolaminess, N-acyl glycines, N-acyl glycyl serines, phosphatidic acids, phosphatidylcholines, phosphatidylethanolamines, phosphatidylethanols, phosphatidylglycerols, phosphatidylinositols, ceramide phosphoinositols, phosphatidylmethanols, phosphatidylserines, steryl esters, stigmasterols, sulfatides, sulfonolipids, sphingomyelins, sulfoquinovosyl diacylglycerosl, sterols, and triacylglycerols. In some embodiments, the complex lipid particle contains at least 10 natural (e.g., plant, bacteria) lipids belonging to one or more of the sub-classes selected from the group consisting of the sub-classes listed above. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, or at least 800 natural lipids belonging to one or more of the sub-classes selected from the group consisting of the sub-classes listed above.

The complex lipid particle may contain 10 or more natural lipids belonging to one or more of the sub-classes selected from the group consisting of acylsterylglycosides, ceramides, digalactosyldiacylglycerols, diacylglyceryl glucuronides, hemibismonoacylglycerophosphates, hexosylceramides, lysophophatidylcholines, lysophosphatidylethanolamines, monogalactosyldiacylglycerols, phosphatidylcholines, phosphatidylethanolamines, phosphatidylethanols, phosphatidylglycerols, phosphatidylinositols, sulfoquinovosyl diacylglycerosl, and sterols. For instance, the complex lipid particle contains at least 15, at least 20, at least 25, at least 30, at least 35, at least 40, at least 45, at least 50, at least 60, at least 70, at least 80, at least 90, at least 100, at least 150, at least 200, at least 250, at least 300, at least 400, at least 400, at least 500, at least 600, at least 700, or at least 800 natural lipids belonging to one or more of the sub-classes selected from the group consisting of the sub-classes listed above.

The complex lipid particle may contain natural lipids from at least 5, at least 6, at least 7, at least 8, at least 9, or at least 10 different classes or sub-classes of lipids from the natural sources. In some embodiments, the complex lipid particle contains natural lipids from at least 5, at least 6, at least 7, at least 8, at least 9, or at least 10 different classes or sub-classes of lipids from a single natural source (e.g., from only the plant source, or from only the bacteria source). In some embodiments, the CLP may contain natural lipids from only one class or only one sub-class of lipids from the natural sources.

The identity (and class and subclass) and the amounts of the lipids extracted from the natural source(s) can be analyzed by lipidomics analysis by solubilizing the lipid extracts or complex lipid particles in compatible solvents and analyzing by a mass spectrometry (e.g., MS/MS). Other known methods, such as charged aerosol detection (CAD) (e.g., HPLC-CAD, normal-phase high-performance liquid chromatography (NP-HPLC-CAD), or reversed-phase high-performance liquid chromatography (RP-HPLC-CAD)), may also be used.

The complex lipid particle may comprise all or a fraction of the lipid species present in the lipid structure from the particular natural source(s), e.g., it may contain at least 10%, at least 20%, at least 30%, at least 40%, at least 50%, at least 60%, at least 70%, at least 80%, at least 90%, or virtually 100% of the lipid species present in the lipid structure from the particular natural source(s).

The complex lipid particle may comprise reduced or minimized protein matter endogenous to the one or more natural sources. For instance, the complex lipid particle may contain less than 50% w/w, less than 45% w/w, less than 40% w/w, less than 35% w/w, less than 30% w/w, less than 25% w/w, less than 20% w/w, less than 15% w/w, less than 10% w/w, less than 9% w/w, less than 8% w/w, less than 7% w/w, less than 6% w/w, less than 5% w/w, less than 4% w/w, less than 3% w/w, less than 2% w/w, less than 1% w/w, less than 0.5% w/w, less than 0.1% w/w, or essentially free of protein matter endogenous to the one or more natural sources. In some instances, the lipid bilayer of the complex lipid particle does not contain proteins. To calculate % w/w of residual protein matter endogenous to the one or more natural sources, protein concentration is divided by the concentration of the natural lipid extract and then multiplied by 100. Alternatively, % w/w is calculated as the percent of the mass of total protein endogenous to the one or more natural sources based on the mass of the total lipid extract.

The complex lipid particle may comprise reduced or minimized residual dsDNA matter endogenous to the one or more natural sources. For instance, the complex lipid particle may contain less than 15% w/w, less than 10% w/w, less than 5% w/w, less than 1% w/w, less than 0.5% w/w, less than 0.1% w/w, less than 0.05% w/w, less than 0.01% w/w, less than 0.005% w/w, less than 0.001% w/w, or essentially free of residual dsDNA matter endogenous to the one or more natural sources. In some instances, the lipid bilayer of the complex lipid particle does not contain residual dsDNA. To calculate % w/w of residual dsDNA matter endogenous to the one or more natural sources, total adjusted dsDNA concentration is divided by the concentration of the natural lipid extract and then multiplied by 100. Alternatively, % w/w is calculated as the percent of the mass of total residual dsDNA endogenous to the one or more natural sources based on the mass of the total lipid extract.

In some embodiments, the complex lipid particle further incorporates a synthetic structural lipid such as a neutral lipid. In some embodiments, the structural lipid component of the complex lipid particle may comprise between 10% w/w and 99% w/w structural lipids derived from a synthetic lipid structure, e.g., it may contain at least 10% w/w, at least 20% w/w, at least 30% w/w, at least 40% w/w, at least 50% w/w, at least 60% w/w, at least 70% w/w, at least 80% w/w, at least 90% w/w, at least 95% w/w, or about 99% w/w lipids derived from a synthetic lipid structure.

In addition to the exogenous ionizable lipid, the complex lipid particle may further comprise at least two other exogenous lipids. The complex lipid particle may include at least 1% w/w, at least 2% w/w, at least 5% w/w, at least 10% w/w, at least 15% w/w, at least 20% w/w, at least 25% w/w, at least 30% w/w, at least 40% w/w, at least 50% w/w, at least 60% w/w, at least 70% w/w, at least 80% w/w, at least 90% w/w, or about 95% w/w exogenous lipids. Exemplary exogenous lipids include ionizable lipids, synthetic structural lipids, sterols, and PEG-lipid conjugate. The complex lipid particle may further comprise at least two exogenous lipids. In some embodiments, the complex lipid particle contains an ionizable lipid, a sterol, and PEG-lipid conjugate. Additional exogenous lipids suitable for being included in the complex lipid particle are described herein below.

In some embodiments, the CLPs contain natural lipids comprising fatty acid-derived tails, said fatty acid-derived tails of the natural lipids being:
about 5 to 20% of fatty acid 16:0
about 0 to 10% of fatty acid 18:1 (C9)
about 0 to 10% of fatty acid 18:1 (C7)
about 5 to 30% of fatty acid 18:2
about 2 to 20% of fatty acid 18:3.

In some embodiments, the CLPs contain phosphatidylcholine (PC) lipids comprising fatty acid-derived tails, said fatty acid-derived tails of the PC lipids being:
about 10 to 20% of fatty acid 16:0
about 2 to 5% of fatty acid 18:0
about 7 to 15% of fatty acid 18:1
about 50 to 75% of fatty acid 18:2
about 2 to 10% of fatty acid 18:3.

In some embodiments, the CLPs contain phosphatidylethanolamines (PE) lipids comprising fatty acid-derived tails, said fatty acid-derived tails of the PE lipids being:
about 0.25 to 5% of fatty acid 14:0
about 25 to 45% of fatty acid 16:0
about 5 to 15% of fatty acid 16:1
about 10 to 25% of fatty acid 17:0
about 25 to 45% of fatty acid 18:1
about 2 to 7% of fatty acid 19:0.

In some embodiments, the CLPs contain natural lipids belonging to the sub-classes of phosphatidylethanolamines, phosphatidylglycerol, and cardiolipin, and comprising:
about 50 to 75 wt/wt % of phosphatidylethanolamines (PE)
about 15 to 30 wt/wt % of phosphatidylglycerol (PG)
about 5 to 15 wt/wt % of cardiolipin (CL).

In some embodiments, the CLPs contain natural lipids comprising
about 10 to 50 wt/wt % of phosphatidylcholines (PC)
about 5 to 50 wt/wt % of phosphatidylethanolamines (PE)
about 0 to 15 wt/wt % of triacylglycerol (TG)
about 5 to 35 wt/wt % of hexosylceramides (HexCer)
about 0 to 5 wt/wt % of phosphatidylglycerol (PG)
about 0 to 7 wt/wt % of phosphatidylserines (PS)
about 0 to 10 wt/wt % of phosphatidylinositols (PI)
about 0 to 5 wt/wt % of cardiolipin (CL).

In some embodiments, the complex lipid particle contains less than 12% w/w of chloroplast endogenous to the one or more natural sources. In some embodiments, the complex lipid particle contains less than 20% w/w, less than 15% w/w, less than 10% w/w, less than 5% w/w, less than 1% w/w, less than 0.5% w/w, or less than 0.1% w/w of chloroplast endogenous to the one or more natural sources.

In some embodiments, the complex lipid particle contains less than 5% w/w of exogenous antioxidant.

In some embodiments, the CLPs contain natural lipids comprising about 0 to 20 wt/wt % of cardiolipin (CL).

### Natural Messenger Packs (NMPs)

A plurality of NMPs in a modified NMP formulation may be loaded with the exogenous peptide, polypeptide, or protein such that at least 5%, at least 10%, at least 15%, at least 25%, at least 50%, at least 75%, at least 90%, or at least 95% of NMPs in the plurality of NMPs encapsulate the exogenous peptide, polypeptide, or protein. In some embodiments, the NMP is derived from an arthropod, fungi, archaea, plant, or bacteria. For instance, one example of NMP derived from a plant source is a plant NMP, which may be referred to as a PMP, which is a lipid (e.g., lipid bilayer, unilamellar, or multilamellar structure) structure that includes a plant EV, or segment, portion, or extract (e.g., lipid extract) thereof. Additional descriptions about PMPs can be found in the section “Plant Messenger Pack (PMPs),” in PCT Application No. PCT/US22/47107, filed on Oct. 19, 2022, which is incorporated herein by reference in its entirety.

NMPs can include Arthropod, Plant, Fungi, Archaea, or Bacteria EVs, or segments, portions, or extracts, thereof, in which the EVs are about 5-2000 nm in diameter. For example, the NMP can include an EV, or segment, portion, or extract thereof, that has a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1250 nm, about 1250-1500 nm, about 1500-1750 nm, or about 1750-2000 nm. In some instances, the NMP includes a Arthropod, Plant, Fungi, Archaea, or Bacteria EV, or segment, portion, or extract thereof, that has a mean diameter of about 5-1400 nm, 5-950 nm, about 5-900 nm, about 5-850 nm, about 5-800 nm, about 5-750 nm, about 5-700 nm, about 5-650 nm, about 5-600 nm, about 5-550 nm, about 5-500 nm, about 5-450 nm, about 5-400 nm, about 5-350 nm, about 5-300 nm, about 5-250 nm, about 5-200 nm, about 5-150 nm, about 5-100 nm, about 5-50 nm, or about 5-25 nm. In certain instances, the Arthropod, Plant, Fungi, Archaea, or Bacteria EV, or segment, portion, or extract thereof, has a mean diameter of about 50-200 nm. In certain instances, the EV, or segment, portion, or extract thereof, has a mean diameter of about 50-300 nm. In certain instances, the EV, or segment, portion, or extract thereof, has a mean diameter of about 200-500 nm. In certain instances, the EV, or segment, portion, or extract thereof, has a mean diameter of about 30-150 nm.

In some instances, the NMP may include a Arthropod, Plant, Fungi, Archaea, or Bacteria EV, or segment, portion, or extract thereof, that has a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1000 nm, or at least 1300. In some instances, the NMP includes a Arthropod, Fungi, Archaea, or Bacteria EV, or segment, portion, or extract thereof, that has a mean diameter less than 1400 nm, less than 1000 nm, less than 950 nm, less than 900 nm, less than 850 nm, less than 800 nm, less than 750 nm, less than 700 nm, less than 650 nm, less than 600 nm, less than 550 nm, less than 500 nm, less than 450 nm, less than 400 nm, less than 350 nm, less than 300 nm, less than 250 nm, less than 200 nm, less than 150 nm, less than 100 nm, or less than 50 nm. A variety of methods (e.g., a dynamic light scattering method) standard in the art can be used to measure the particle diameter of the EVs, or segment, portion, or extract thereof.

In some instances, the NMP may include an Arthropod, Plant, Fungi, Archaea, or Bacteria EV, or segment, portion, or extract thereof, that has a mean surface area of 77 nm² to 3.2×10⁶ nm² (e.g., 77-100 nm², 100-1000 nm², 1000-1×10⁴ nm², 1×10⁴-1×10⁵ nm², 1×10⁵-1×10⁶ nm², or 1×10⁶-3.2×10⁶ nm²). In some instances, the NMP may include a Arthropod, Fungi, Archaea, or Bacteria EV, or segment, portion, or extract thereof, that has a mean volume of 65 nm³ to 5.3×10⁸ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-5.3×10⁸ nm³). In some instances, the NMP may include a Arthropod, Plant, Fungi, Archaea, or Bacteria EV, or segment, portion, or extract thereof, that has a mean surface area of at least 77 nm², (e.g., at least 77 nm², at least 100 nm², at least 1000 nm², at least 1×10⁴ nm², at least 1×10⁵ nm², at least 1×10⁶ nm², or at least 2×10⁶ nm²). In some instances, the NMP may include a Arthropod, Fungi, Archaea, or Bacteria EV, or segment, portion, or extract thereof, that has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 2×10⁸ nm³, at least 3×10⁸ nm³, at least 4×10⁸ nm³, or at least 5×10⁸ nm³.

In some instances, the NMP can have the same size as the Arthropod, Plant, Fungi, Archaea, or Bacteria EV or segment, extract, or portion thereof. Alternatively, the NMP may have a different size than the initial EV from which the NMP is produced. For example, the NMP may have a diameter of about 5-2000 nm in diameter. For example, the NMP can have a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1200 nm, about 1200-1400 nm, about 1400-1600 nm, about 1600-1800 nm, or about 1800-2000 nm. In some instances, the NMP may have a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1000 nm, at least 1200 nm, at least 1400 nm, at least 1600 nm, at least 1800 nm, or about 2000 nm. A variety of methods (e.g., a dynamic light scattering method) standard in the art can be used to measure the particle diameter of the NMPs. In some instances, the size of the NMP is determined following loading of heterologous functional agents, or following other modifications to the NMPs.

In some instances, the NMP may have a mean surface area of 77 nm² to 1.3×10⁷ nm² (e.g., 77-100 nm², 100-1000 nm², 1000-1×10⁴ nm², 1×10⁴-1×10⁵ nm², 1×10⁵-1×10⁶ nm², or 1×10⁶-1.3×10⁷ nm²). In some instances, the NMP may have a mean volume of 65 nm³ to 4.2×10⁹ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-1×10⁹ nm³, or 1×10⁹-4.2×10⁹ nm³). In some instances, the NMP has a mean surface area of at least 77 nm², (e.g., at least 77 nm², at least 100 nm², at least 1000 nm², at least 1×10⁴ nm², at least 1×10⁵ nm², at least 1×10⁶ nm², or at least 1×10⁷ nm²). In some instances, the NMP has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 1×10⁹ nm³, at least 2×10⁹ nm³, at least 3×10⁹ nm³, or at least 4×10⁹ nm³).

In some instances, the NMP may include an intact Arthropod, Plant, Fungi, Archaea, or Bacteria EV. In some embodiments, the NMP may include a non-plant natural source such as bacterial or animal-derived organs EV, or segment, portion, or extract thereof. Alternatively, the NMP may include a segment, portion, or extract of the full surface area of the vesicle (e.g., a segment, portion, or extract including less than 100% (e.g., less than 90%, less than 80%, less than 70%, less than 60%, less than 50%, less than 40%, less than 30%, less than 20%, less than 10%, less than 10%, less than 5%, or less than 1%) of the full surface area of the vesicle) of a EV. The segment, portion, or extract may be any shape, such as a circumferential segment, spherical segment (e.g., hemisphere), curvilinear segment, linear segment, or flat segment. In instances where the segment is a spherical segment of the vesicle, the spherical segment may represent one that arises from the splitting of a spherical vesicle along a pair of parallel lines, or one that arises from the splitting of a spherical vesicle along a pair of non-parallel lines. Accordingly, the plurality of NMPs can include a plurality of intact EVs, a plurality of EV segments, portions, or extracts, or a mixture of intact and segments of EVs. One skilled in the art will appreciate that the ratio of intact to segmented EVs will depend on the particular isolation method used. For example, grinding or blending an Arthropod, Fungi, Plant, Archaea, or Bacteria, or part thereof, may produce NMPs that contain a higher percentage of EV segments, portions, or extracts than a non-destructive extraction method, such as vacuum-infiltration.

In instances where, the NMP includes a segment, portion, or extract of a Arthropod, Fungi, Archaea, or Bacteria EV, the EV segment, portion, or extract may have a mean surface area less than that of an intact vesicle, e.g., a mean surface area less than 77 nm², 100 nm², 1000 nm², 1×10⁴ nm², 1×10⁵ nm², 1×10⁶ nm², or 3.2×10⁶ nm²). In some instances, the EV segment, portion, or extract has a surface area of less than 70 nm², 60 nm², 50 nm², 40 nm², 30 nm², 20 nm², or 10 nm²). In some instances, the NMP may include a Arthropod, Fungi, Archaea, or Bacteria EV, or segment, portion, or extract thereof, that has a mean volume less than that of an intact vesicle, e.g., a mean volume of less than 65 nm³, 100 nm³, 1000 nm³, 1×10⁴ nm³, 1×10⁵ nm³, 1×10⁶ nm³, 1×10⁷ nm³, 1×10⁸ nm³, or 5.3×10⁸ nm³).

In instances where the NMP includes an extract of a Arthropod, Plant, Fungi, Archaea, or Bacteria EV, e.g., in instances where the NMP includes lipids extracted (e.g., with chloroform or ethanol) from a EV, the NMP may include at least 1%, 2%, 5%, 10%, 20%, 30%, 40%, 50%, 60%, 70%, 80%, 90%, 95%, 99%, or more than 99% of lipids extracted (e.g., with chloroform or with ethanol) from a Arthropod, Fungi, Archaea, or Bacteria EV. The NMPs in the plurality may include Arthropod, Plant, Fungi, Archaea, or Bacteria EV segments and/or EV-extracted lipids or a mixture thereof.

### Production of NMPs

NMPs may be produced from Arthropod, Fungi, Plant, Archaea, or Bacteria EVs, or a segment, portion or extract (e.g., lipid extract) thereof, that occur naturally in Arthropod, Fungi, Plant, Archaea, or Bacteria, or parts thereof, including tissues or cells. In some embodiments, the NMP may include a non-plant natural source such as bacterial or animal-derived organs EV, or segment, portion, or extract thereof. An exemplary method for producing NMPs includes (a) providing an initial sample from a source, or a part thereof, wherein the source or part thereof comprises EVs; and (b) isolating a crude NMP fraction from the initial sample, wherein the crude NMP fraction has a decreased level of at least one contaminant or undesired component from the source or part thereof relative to the level in the initial sample. The method can further include an additional step (c) comprising purifying the crude NMP fraction, thereby producing a plurality of pure NMPs, wherein the plurality of pure NMPs have a decreased level of at least one contaminant or undesired component from the Arthropod, Fungi, Archaea, or Bacteria or part thereof relative to the level in the crude EV fraction. Each production step is discussed in further detail, below.

For instance, PMPs may be produced from plant EVs, or a segment, portion or extract (e.g., lipid extract) thereof, that occur naturally in plants, or parts thereof, including plant tissues or plant cells. An exemplary method for producing PMPs includes (a) providing an initial sample from a plant, or a part thereof, wherein the plant or part thereof comprises EVs; and (b) isolating a crude PMP fraction from the initial sample, wherein the crude PMP fraction has a decreased level of at least one contaminant or undesired component from the plant or part thereof relative to the level in the initial sample. The method can further include an additional step (c) comprising purifying the crude PMP fraction, thereby producing a plurality of pure PMPs, wherein the plurality of pure PMPs have a decreased level of at least one contaminant or undesired component from the plant or part thereof relative to the level in the crude EV fraction. Each production step is discussed in further detail, below. Exemplary methods regarding the isolation and purification of NMPs (e.g., plant NMPs, PMPs) are found, for example, in Rutter and Innes, Plant Physiol. 173(1): 728-741, 2017; Rutter et al, Bio. Protoc. 7(17): e2533, 2017; Regente et al, J of Exp. Biol. 68(20): 5485-5496, 2017; Mu et al, Mol. Nutr. Food Res., 58, 1561-1573, 2014, and Regente et al, FEBS Letters. 583: 3363-3366, 2009, each of which is herein incorporated by reference. Additional descriptions about the production of PMPs can be found in the section “Production of PMPs,” in PCT Application No. PCT/US22/47107, filed on Oct. 19, 2022, which is incorporated herein by reference in its entirety.

For example, a plurality of NMPs may be isolated from a Arthropod, Fungi, Plant, Archaea, or Bacteria by a process which includes the steps of: (a) providing an initial sample from a source, or a part thereof, wherein the source or part thereof comprises EVs; (b) isolating a crude NMP fraction from the initial sample, wherein the crude NMP fraction has a decreased level of at least one contaminant or undesired component from the Arthropod, Fungi, Plant, Archaea, or Bacteria or part thereof relative to the level in the initial sample (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%); and (c) purifying the crude NMP fraction, thereby producing a plurality of pure NMPs, wherein the plurality of pure NMPs have a decreased level of at least one contaminant or undesired component from the source or part thereof relative to the level in the crude EV fraction (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%).

The NMPs provided herein can include an Arthropod, Fungi, Archaea, or Bacteria EV, or segment, portion, or extract thereof, isolated from a variety of sources.

For instance, the plant NMPs, PMPs, can include a plant EV, or segment, portion, or extract thereof, produced from a variety of plants. PMPs may be produced from any genera of plants (vascular or nonvascular), including but not limited to angiosperms (monocotyledonous and dicotyledonous plants), gymnosperms, ferns, selaginellas, horsetails, psilophytes, lycophytes, algae (e.g., unicellular or multicellular, e.g., archaeplastida), or bryophytes. In certain instances, PMPs can be produced using a vascular plant, for example monocotyledons or dicotyledons or gymnosperms. For example, PMPs can be produced using alfalfa, apple, Arabidopsis, banana, barley, a Brassica species (e.g., Arabidopsis thaliana or Brassica napus), canola, castor bean, chicory, chrysanthemum, clover, cocoa, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat or vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes, kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, or wheat.

PMPs (i.e., plant NMPs) may be produced using a whole plant (e.g., a whole rosettes or seedlings) or alternatively from one or more plant parts (e.g., leaf, seed, root, fruit, vegetable, pollen, phloem sap, or xylem sap). For example, PMPs can be produced using shoot vegetative organs/structures (e.g., leaves, stems, or tubers), roots, flowers and floral organs/structures (e.g., pollen, bracts, sepals, petals, stamens, carpels, anthers, or ovules), seed (including embryo, endosperm, or seed coat), fruit (the mature ovary), sap (e.g., phloem or xylem sap), plant tissue (e.g., vascular tissue, ground tissue, tumor tissue, or the like), and cells (e.g., single cells, protoplasts, embryos, callus tissue, guard cells, egg cells, or the like), or progeny of same. For instance, the isolation step may involve (a) providing a plant, or a part thereof. In some examples, the plant part is an Arabidopsis leaf. The plant may be at any stage of development. For example, the PMPs can be produced using seedlings, e.g., 1 week, 2 week, 3 week, 4 week, 5 week, 6 week, 7 week, or 8 week old seedlings (e.g., Arabidopsis seedlings). Other exemplary PMPs can include PMPs produced using roots (e.g., ginger roots), fruit juice (e.g., grapefruit juice), vegetables (e.g., broccoli), pollen (e.g., olive pollen), phloem sap (e.g., Arabidopsis phloem sap), or xylem sap (e.g., tomato plant xylem sap). In some embodiments, the PMPs are produced from algae or lemon.

NMPs may be isolated from any genera of Arthropod, Fungi, Archaea, or Bacteria, including but not limited to crabs, crawfish, shrimp, spiders, scorpions, crickets, grasshoppers, beetles, millipedes, ticks, mites, centipedes, ants, wasps, dragonflies, flies, gnats, other insects and crustaceans, yeast, mushrooms, puffballs, stinkhorns, boletes, smuts, bunts, bracket fungi, jelly fungi, toadstools, molds, rusts, earth stars, chanterelles, ergot, pyrolobus, picrophilus, methanogens, crenarchaeota, nanoarchaeota, ignicoccus, cenarchaeum, halophiles, Escherichia, Acinetobacter, Agrobacterium, Anabaena, Anaplasma, Aquifex, Azoarcus, Azospirillum, Azotobacter, Bartonella, Bordetella, Bradyrhizobium, Brucella, Buchnera, Burkholderia, Candidatus, Chromobacterium, Coxiella, Crocosphaera, Dechloromonas, Desulfitobacterium, Desulfotalea, Erwinia, Francisella, Fusobacterium, Gloeobacter, Gluconobacter, Helicobacter, Legionella, Magnetospirillum, Mesorhizobium, Methylobacterium, Methylococcus, Neisseria, Nitrosomonas, Nostoc, Photobacterium, Photorhabdus, Phyllobacterium, Polaromonas, Prochlorococcus, Pseudomonas, Psychrobacter, Ralstonia, Rubrivivax, Salmonella, Shewanella, Shigella, Sinorhizobium, Synechococcus, Synechocystis, Thermosynechococcus, Thermotoga, Thermus, Thiobacillus, Trichodesmium, Vibrio, Wigglesworthia, Wolinella, Xanthomonas, Xylella, Yersinia, Bacillus, Bifidobacterium, Clostridium, Corynebacterium, Deinococcus, Enterococcus, Exiguobacterium, Geobacillus, Lactobacillus, Listeria, Leuconostoc, Moorella, Oceanobacillus, Rhizobium, Rickettsia, Staphylococcus, Streptococcus, Symbiobacterium, or Thermoanaerobacter.

NMPs may be produced from a whole Arthropod, Fungi, Archaea, or Bacteria (e.g., a whole insect, spider, crustacean, fungi, or single-cell of archaea or bacteria) or alternatively from one or more source parts (e.g., segments, organs, eggs, spores, mycelium, tissue, membrane or cell wall). For example, NMPs can be produced from organs/structures/tissues/cell cultures (e.g., body segments, appendages, organs, eggs, exoskeleton, embryos, spores, mycelium, hyphae, thallus, suspension cultures, cell walls, inner or outer membranes, gametophytes, sporophytes, polymerases, glycerol-ether lipids, metabolic products, flagella, pili, ribosomes or organelles) or progeny of same. The source may be at any stage of development. In some embodiments, the NMP is produced from an insect or fungi, (e.g. cricket, yeast, or mushroom). In some embodiments, the NMP is produced from a bacteria or archaea (e.g. E. coli). In some embodiments, the NMP is produced from algae (e.g. kelp or chlorella). In some embodiments, the NMP is produced from an animal organ (e.g. brain or blood).

NMPs can be produced from a plant, Arthropod, Fungi, Archaea, or Bacteria, or part thereof, by a variety of methods. Any method that allows release of the EV-containing fraction of a source, or an otherwise extracellular fraction that contains NMPs comprising secreted EVs (e.g., cell culture media) is suitable in the present methods. EVs can be separated from the source or source part by either destructive (e.g., grinding or blending) or non-destructive (washing or vacuum infiltration) methods. For instance, the plant, Arthropod, Fungi, Archaea, or Bacteria, or part thereof, can be vacuum-infiltrated, ground, blended, or a combination thereof to isolate EVs from the source or source part, thereby producing NMPs. For instance, the isolating step may involve (b) isolating a crude NMP fraction from the initial sample (e.g., a plant, Arthropod, Fungi, Archaea, or Bacteria or part, or a sample derived from a plant, Arthropod, Fungi, Archaea, or Bacteria or part), wherein the crude NMP fraction has a decreased level of at least one contaminant or undesired component from the source or part thereof relative to the level in the initial sample; wherein the isolating step involves vacuum infiltrating the plant, Arthropod, Fungi, Archaea, or Bacteria (e.g., with a vesicle isolation buffer) to release and collect the desired fraction. Alternatively, the isolating step may involve (b) grinding or blending the source to release the EVs, thereby producing NMPs.

Upon isolating the plant, Arthropod, Fungi, Archaea, or Bacteria EVs, thereby producing NMPs, the NMPs can be separated or collected into a crude NMP fraction. For instance, the separating step may involve separating the plurality of NMPs into a crude NMP fraction using centrifugation (e.g., differential centrifugation or ultracentrifugation) and/or filtration to separate the NMP-containing fraction from large contaminants, including tissue debris, cells, or cell organelles. As such, the crude NMP fraction will have a decreased number of large contaminants, including, for example, tissue debris, cells, or cell organelles (e.g., nuclei, mitochondria, etc.), as compared to the initial sample from the source or source part.

The crude NMP fraction can be further purified by additional purification methods to produce a plurality of pure NMPs. For example, the crude NMP fraction can be separated from other source components by ultracentrifugation, e.g., using a density gradient (iodixanol or sucrose), size-exclusion, and/or use of other approaches to remove aggregated components (e.g., precipitation or size-exclusion chromatography). The resulting pure NMPs may have a decreased level of contaminants or undesired components from the source (e.g., one or more non-NMP components, such as protein aggregates, nucleic acid aggregates, protein-nucleic acid aggregates, free lipoproteins, lipido-proteic structures), nuclei, cell wall components, cell organelles, or a combination thereof) relative to one or more fractions generated during the earlier separation steps, or relative to a pre-established threshold level, e.g., a commercial release specification. For example, the pure NMPs may have a decreased level (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) of source organelles or cell wall components relative to the level in the initial sample. In some instances, the pure NMPs are substantially free (e.g., have undetectable levels) of one or more non-NMP components, such as protein aggregates, nucleic acid aggregates, protein-nucleic acid aggregates, free lipoproteins, lipido-proteic structures), nuclei, cell wall components, cell organelles, or a combination thereof. Further examples of the releasing and separation steps can be found in WO 2021/041301. The NMPs may be at a concentration of, e.g., 1×10⁹, 5×10⁹, 1×10¹⁰, 5×10¹⁰, 5×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, or more than 1×10¹³ NMPs/mL.

For example, protein aggregates may be removed from isolated NMPs. For example, the isolated NMP solution can be taken through a range of pHs (e.g., as measured using a pH probe) to precipitate out protein aggregates in solution. The pH can be adjusted to, e.g., pH 3, pH 5, pH 7, pH 9, or pH 11 with the addition of, e.g., sodium hydroxide or hydrochloric acid. Once the solution is at the specified pH, it can be filtered to remove particulates. Alternatively, the isolated NMP solution can be flocculated using the addition of charged polymers, such as Polymin-P or Praestol 2640. Briefly, Polymin-P or Praestol 2640 is added to the solution and mixed with an impeller. The solution can then be filtered to remove particulates. Alternatively, aggregates can be solubilized by increasing salt concentration. For example, NaCl can be added to the isolated NMP solution until it is at, e.g., 1 mol/L. The solution can then be filtered to isolate the NMPs. Alternatively, aggregates are solubilized by increasing the temperature. For example, the isolated NMPs can be heated under mixing until the solution has reached a uniform temperature of, e.g., 50° C. for 5 minutes. The NMP mixture can then be filtered to isolate the NMPs. Alternatively, soluble contaminants from NMP solutions can be separated by size-exclusion chromatography column according to standard procedures, where NMPs elute in the first fractions, whereas proteins, ribonucleoproteins, and some lipoproteins are eluted later. The efficiency of protein aggregate removal can be determined by measuring and comparing the protein concentration before and after removal of protein aggregates via BCA/Bradford protein quantification. In some embodiments, protein aggregates are removed before the exogenous peptide, polypeptide, or protein is encapsulated by the NMP. In other embodiments, protein aggregates are removed after the exogenous peptide, polypeptide, or protein is encapsulated by the NMP.

In some embodiments, the preparation of NMPs from natural sources is through an ethanol extraction method. In some aspects, a 3:2 ethyl acetate:ethanol solvent aids in extraction. In some embodiments, the preparation of NMPs from natural sources is through a modified Matyash extraction method. In some aspects, a 1:2 MeOH:MTBE solvent aids in extraction.

Any of the production methods described herein can be supplemented with any quantitative or qualitative methods known in the art to characterize or identify the NMPs at any step of the production process. NMPs may be characterized by a variety of analysis methods to estimate NMP yield, NMP concentration, NMP purity, NMP composition, or NMP sizes. NMPs can be evaluated by a number of methods known in the art that enable visualization, quantitation, or qualitative characterization (e.g., identification of the composition) of the NMPs, such as microscopy (e.g., transmission electron microscopy), dynamic light scattering, nanoparticle tracking, spectroscopy (e.g., Fourier transform infrared analysis), or mass spectrometry (protein and lipid analysis). In certain instances, methods (e.g., mass spectroscopy) may be used to identify EV markers present on the NMP. To aid in analysis and characterization, of the NMP fraction, the NMPs can additionally be labelled or stained. For example, the NMPs can be stained with 3,3′-dihexyloxacarbocyanine iodide (DIOC₆), a fluorescent lipophilic dye, PKH67 (Sigma Aldrich); Alexa Fluor®488 (Thermo Fisher Scientific), or DyLight™ 800 (Thermo Fisher). In the absence of sophisticated forms of nanoparticle tracking, this relatively simple approach quantifies the total membrane content and can be used to indirectly measure the concentration of NMPs (Rutter and Innes, Plant Physiol. 173(1): 728-741, 2017; Rutter et al, Bio. Protoc. 7(17): e2533, 2017). For more precise measurements, and to assess the size distributions of NMPs, nanoparticle tracking, nano flow cytometry, or Tunable Resistive Pulse Sensing can be used.

During the production process, the NMPs can optionally be prepared such that the NMPs are at an increased concentration (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) relative to the EV level in a control or initial sample. The isolated NMPs may make up about 0.1% to about 100% of the NMP composition, such as any one of about 0.01% to about 100%, about 1% to about 99.9%, about 0.1% to about 10%, about 1% to about 25%, about 10% to about 50%, about 50% to about 99%. In some instances, the composition described herein includes at least any of 0.1%, 0.5%, 1%, 5%, 10%, 20%, 30%, 40%, 50%, 60%, 70%, 80%, 90%, 95%, or more NMPs, e.g., as measured by wt/vol, percent NMP protein composition, and/or percent lipid composition (e.g., by measuring fluorescently labelled lipids)). In some instances, the concentrated agents are used as commercial products, e.g., the final user may use diluted agents, which have a substantially lower concentration of active ingredient. In some embodiments, the composition described herein is formulated as a NMP concentrate formulation, e.g., an ultra-low-volume concentrate formulation. In some embodiments, the NMPs in the composition are at a concentration effective to increase the fitness of an organism, e.g., a plant, an animal, an insect, a bacterium, or a fungus. In other aspects, the NMPs in the composition are at a concentration effective to decrease the fitness of an organism, e.g., a plant, an animal, an insect, a bacterium, or a fungus.

NMPs can be produced from a variety of Arthropod, Fungi, Plant, Archaea, or Bacteria, or one or more parts thereof (e.g., segments, organs, eggs, spores, mycelium, tissue, membrane or cell wall). For example, NMPs can be produced from organs/structures/tissues/cell cultures (e.g., body segments, appendages, organs, eggs, exoskeleton, embryos, spores, mycelium, hyphae, thallus, suspension cultures, cell walls, inner or outer membranes, gametophytes, sporophytes, polymerases, glycerol-ether lipids, metabolic products, flagella, pili, ribosomes or organelles) or progeny of same. The source may be at any stage of development. In some embodiments, the NMP is produced from an insect or fungi, (e.g. cricket, yeast, or mushroom). In some embodiments, the NMP is produced from a bacteria or archaea (e.g. E. coli). In some embodiments, the NMP is produced from algae (e.g. kelp or chlorella). In some embodiments, the NMP is produced from an animal organ (e.g. brain or blood).

NMPs can be produced and purified by a variety of methods, for example, by using a density gradient (iodixanol or sucrose) in conjunction with ultracentrifugation and/or methods to remove aggregated contaminants, e.g., precipitation or size-exclusion chromatography.

In some instances, the NMPs of the present compositions and methods can be isolated from an Arthropod, Fungi, Archaea, or Bacteria, or part thereof, and used without further modification to the NMP. In other instances, the NMP can be modified prior to use, as outlined further herein. In some instances, the NMPs are PMPs. In some instances, the NMPs of the present compositions and methods can be isolated from a plant, or part thereof, and used without further modification to the NMP. In other instances, the NMP can be modified prior to use, as outlined further herein.

### Lipid Reconstructed Natural Messenger Packs (LNMPs)

A lipid reconstructed NMP (LNMP) is used herein. LNMP refers to a NMP that has been derived from a lipid structure (e.g., a lipid bilayer, unilamellar, multilamellar structure; e.g., a vesicular lipid structure) derived from (e.g., enriched, isolated or purified from) a natural source, wherein the lipid structure is disrupted (e.g., disrupted by lipid extraction) and reassembled or reconstituted in a liquid phase (e.g., a liquid phase containing a cargo) using standard methods, e.g., reconstituted by a method comprising lipid film hydration and/or solvent injection, to produce the LNMP, as is described herein. For instance, a plant LNMP can be referred to as a LPMP, a lipid reconstructed plant messenger pack, which is derived from a plant source.

The method for a lipid reconstructing NMP (e.g., LPMP) may, if desired, further comprise sonication, freeze/thaw treatment, and/or lipid extrusion, e.g., to reduce the size of the reconstituted LNMPs. Alternatively, LNMPs (e.g., LPMPs) may be produced using a microfluidic device (such as a NanoAssemblr® IGNITE™ microfluidic instrument (Precision NanoSystems)).

In some embodiments, the LNMPs (e.g., LPMPs) are produced by a process which comprises the steps of (a) providing a plurality of purified NMPs (e.g., purified PMPs); (b) processing the plurality of NMPs (e.g., PMPs) to produce a lipid film; (c) reconstituting the lipid film in an organic solvent or solvent combination, thereby producing a lipid solution; and (d) processing the lipid solution of step (c) in a microfluidics device comprising an aqueous phase, thereby producing the LNMPs (e.g., LPMPs).

In some instances, processing the plurality of NMPs (e.g., PMPs) to produce a lipid film includes extracting lipids from the plurality of NMPs, e.g., extracting lipids using the Bligh-Dyer method (Bligh and Dyer, J Biolchem Physiol, 37: 911-917, 1959). The extracted lipids may be provided as a stock solution, e.g., a solution in chloroform:methanol. Producing the lipid film may comprise, e.g., evaporation of the solvent with a stream of inert gas (e.g., nitrogen).

### Natural Lipids

A LNMP may comprise between 10% and 100% lipids derived from the lipid structure from the natural source (e.g., lemon or algae), e.g., may contain at least 10%, at least 20%, at least 30%, at least 40%, at least 50%, at least 60%, at least 70%, at least 80%, at least 90%, at least 95%, or 100% lipids derived from the lipid structure from the natural source. A LNMP may comprise all or a fraction of the lipid species present in the lipid structure from the source (e.g., lemon or algae), e.g., it may contain at least 10%, at least 20%, at least 30%, at least 40%, at least 50%, at least 60%, at least 70%, at least 80%, at least 90%, or 100% of the lipid species present in the lipid structure from the source. A LNMP may comprise none, a fraction, or all of the protein species present in the lipid structure from the source (e.g., lemon or algae), e.g., may contain 0%, less than 1%, less than 5%, less than 10%, less than 15%, less than 20%, less than 30%, less than 40%, less than 50%, less than 60%, less than 70%, less than 80%, less than 90%, less than 100%, or 100% of the protein species present in the lipid structure from the natural source (e.g., lemon or algae). In some instances, the lipid bilayer of the LNMP does not contain proteins. In some instances, the lipid structure of the LNMP contains a reduced amount of proteins relative to the lipid structure from the natural source.

In some embodiments, the natural lipids of the LNMPs are extracted from a plant source, such as lemon or algae. In some embodiments, the natural lipids are soy-derived lipids. In some embodiments, the soy-derived lipids comprise soy PC, soy PE, soy PI, soy PA, lyso PC (soy LPC), lyso PI (soy LPI), soy PG, soyl PL (phospholipid) mixture, soy PS, soy LPS, soy polar, or a combination thereof. In some embodiments, the natural lipids of the LNMPs are extracted from a bacteria source, such as Escherichia or Salmonella.

### Exogenous Lipids

The LNMPs may be modified to contain a heterologous agent (e.g., a cell-penetrating agent) that is capable of increasing cell uptake (e.g., animal cell uptake (e.g., mammalian cell uptake, e.g., human cell uptake), plant cell uptake, bacterial cell uptake, or fungal cell uptake) relative to an unmodified LNMP. For example, the modified LNMPs may include (e.g., be loaded with, e.g., encapsulate or be conjugated to) or be formulated with (e.g., be suspended or resuspended in a solution comprising) a cell-penetrating agent, such as an ionizable lipid. Each of the modified LNMPs may comprise at least 1%, 5%, 10%, 20%, 30%, 40%, 50%, 60%, 70%, 80%, 90%, or more than 90% ionizable lipid.

LNMPs may include one or more exogenous lipids, e.g., lipids that are exogenous to the source (e.g., originating from a source that is not the natural source or natural part from which the LNMP is produced). The lipid composition of the LNMP may include 0%, less than 1%, or at least 1%, 2%, 5%, 10%, 15%, 20%, 25%, 30%, 40%, 50%, 60%, 70%, 80%, 90%, 95%, or more than 95% exogenous lipid. In some examples, the exogenous lipid (e.g., ionizable lipid) is added to amount to 25% or 40% (w/w) of total lipids in the preparation. In some examples, the exogenous lipid is added to the preparation prior to step (b), e.g., mixed with extracted NMP lipids prior to step (b).

Exemplary exogenous lipids include ionizable lipids. The ionizable lipids in the LNMP compositions herein include one or more from the compounds of groups i)-iv) as described herein.

Exogenous lipids may also include cationic lipids.

In some instances, the exogenous lipid may also include an ionizable lipid or cationic lipid chosen from 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), DLin-MC3-DMA (MC3), dioleoyl-3-trimethylammonium propane (DODAP), DC-cholesterol, DOTAP, Ethyl PC, GL67, DLin-KC2-DMA (KC2), MD1 (cKK-E12), OF2, EPC, ZA3-Ep10, TT3, LP01, 5A2-SC8, Lipid 5 (Moderna), a cationic sulfonamide amino lipid, an amphiphilic zwitterionic amino lipid, DODAC, DOBAQ, YSK05, DOBAT, DOBAQ, DOPAT, DOMPAQ, DOAAQ, DMAP-BLP, DLinDMA, DODMA, DOTMA, DSDMA, DOSPA, DODAC, DOBAQ, DMRIE, DOTAP-cholesterol, GL67A, and 98N12-5 or a combination thereof.

In some embodiments, the exogenous lipid may also include an ionizable lipid or cationic lipid chosen from C12-200, MC3, DODAP, DC-cholesterol, DOTAP, Ethyl PC, GL67, KC2, MD1, OF2, EPC, ZA3-Ep10, TT3, LP01, 5A2-SC8, Lipid 5 (Moderna), a cationic sulfonamide amino lipid, and an amphiphilic zwitterionic amino lipid or a combination thereof. In some embodiments, the ionizable lipid is chosen from C12-200, MC3, DODAP, and DC-cholesterol or combinations thereof. In some instances, the ionizable lipid is an ionizable lipid. In some embodiments, the ionizable lipid is 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200) or (6Z,9Z,28Z,31Z)-Heptatriaconta-6,9,28,31-tetraen-19-yl 4-(dimethylamino)butanoate, DLin-MC3-DMA (MC3). In some instances, the exogenous lipid is a cationic lipid. In some embodiments, the cationic lipid is DC-cholesterol or dioleoyl-3-trimethylammonium propane (DOTAP).

In some instances, the LNMPs comprise at least 1%, 5%, 10%, 20%, 30%, 40%, 50%, 60%, 70%, 80%, 90%, or more than 90% ionizable lipid.

In some instances, the LNMPs comprise a molar ratio of least 0.1%, 1%, 5%, 10%, 15%, 20%, 25%, 30%, 35%, 40%, 45%, 50%, 55%, 60%, 65%, 70%, 75% 80%, 85%, 90%, or more than 90% ionizable lipid, e.g., 1%-10%, 10%-20%, 20%-30%, 30%-40%, 40%-50%, 50%-60%, 60%-70%, 70%-80%, or 80%-90% ionizable lipid, e.g., about 30%-75% ionizable lipid (e.g., about 30%-75% ionizable lipid). In some embodiments, the LNMP comprises 25% C12-200. In some embodiments, the LNMP comprises a molar ratio of 35% C12-200. In some embodiments, the LNMP comprises a molar ratio of 50% C12-200. In some embodiments, the LNMP comprises 40% MC3. In some embodiments, the LNMP comprises a molar ratio of 50% C12-200. In some embodiments, the LNMP comprises 20% or 40% DC-cholesterol. In some embodiments, the LNMP comprises 25% or 40% DOTAP.

The agent may increase uptake of the LNMP as a whole or may increase uptake of a portion or component of the LNMP (e.g., the nucleic acid vaccine) carried by the LNMP. The degree to which cell uptake is increased may vary depending on the natural source or natural source part to which the composition described herein is delivered, the LNMP formulation, and other modifications made to the LNMP, For example, the modified LNMPs may have an increased cell uptake (e.g., animal cell uptake, plant cell uptake, bacterial cell uptake, or fungal cell uptake) of at least 1%, 2%, 5%, 10%, 15%, 20%, 30%, 40%, 50%, 60%, 70%, 80%, 90%, or 100% relative to an unmodified LNMP. In some instances, the increased cell uptake is an increased cell uptake of at least 2×-fold, 4×-fold, 5×-fold, 10×-fold, 100×-fold, or 1000×-fold relative to an unmodified LNMP.

In some embodiments, a LNMP that has been modified with an ionizable lipid more efficiently encapsulates a negatively charged a polynucleotide than a LNMP that has not been modified with an ionizable lipid. In some aspects, a LNMP that has been modified with an ionizable lipid has altered biodistribution relative to a LNMP that has not been modified with an ionizable lipid. In some aspects, a LNMP that has been modified with an ionizable lipid has altered (e.g., increased) fusion with an endosomal membrane of a target cell relative to a LNMP that has not been modified with an ionizable lipid.

### Ionizable Lipids

In some embodiments, the ionizable lipid has at least one (e.g., one, two, three, four or all five) of the characteristics listed below:
(i) at least 2 ionizable amines (e.g., at least 2, at least 3, at least 4, at least 5, at least 6, or more than 6 ionizable amines, e.g., 2, 3, 4, 5, 6, 7, 8, 9, 10, 11, 12, or more than 12 ionizable amines);
(ii) at least 3 lipid tails (e.g., at least 3, at least 4, at least 5, at least 6, or more than 6 lipid tails, e.g., 2, 3, 4, 5, 6, 7, 8, 9, 10, 11, 12, or more than 12 lipid tails), wherein each of the lipid tails is independently at least 6 carbon atoms in length (e.g., at least 6, at least 7, at least 8, at least 9, at least 10, at least 11, at least 12, at least 13, at least 14, at least 15, at least 16, at least 17, at least 18, or more than 18 carbon atoms in length, e.g., 6, 7, 8, 9, 10, 11, 12, 13, 14, 15, 16, 17, 18, 19, 20, 21, 22, 23, 24, 25, or more than 25 carbon atoms in length);
(iii) an acid dissociation constant (pKa) of from about 4.5 to about 7.5 (e.g., a pKa of about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5 (e.g., a pKa of from about 6.5 and about 7.5 (e.g., a pKa of about 6.5, 6.6, 6.7, 6.8, 6.9, 7.0, 7.1, 7.2, 7.3, 7.4, or 7.5));
(iv) an ionizable amine and a heterorganic group; and
(v) an N:P (amines of ionizable lipid:phosphates of mRNA or circRNA) ratio of at least 3 (or at least 4);

In some embodiments, the ionizable lipid is an ionizable amine and a heterorganic group. In some embodiments, the heterorganic group is hydroxyl. In some embodiments, the heterorganic group comprises a hydrogen bond donor. In some embodiments, the heterorganic group comprises a hydrogen bond acceptor. In some embodiments, the heterorganic group is —OH, —SH, —(CO)H, —CO₂H, —NH₂, —CONH₂, optionally substituted C₁-C₆ alkoxy, or fluorine.

In some embodiments, the ionizable lipid is an ionizable amine and a heterorganic group separated by a chain of at least two atoms.

The ionizable lipid in the LNMP compositions included one of the compounds from group i) to group iv) as discussed below.

### Ionizable Lipid Compounds i)

In some embodiments, the ionizable lipid is represented by the following formula I:

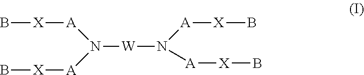

a pharmaceutically acceptable salt thereof, or a stereoisomer of any of the foregoing,
wherein
each A is independently C₁-C₁₆ branched or unbranched alkyl or C₁-C₁₆ branched or unbranched alkenyl, optionally substituted with heteroatom or substituted with OH, SH, or halogen;
each B is independently C₁-C₁₆ branched or unbranched alkyl or C₁-C₁₆ branched or unbranched alkenyl, optionally substituted with heteroatom or substituted with OH, SH, or halogen;
each X is independently a biodegradable moiety; and
W is

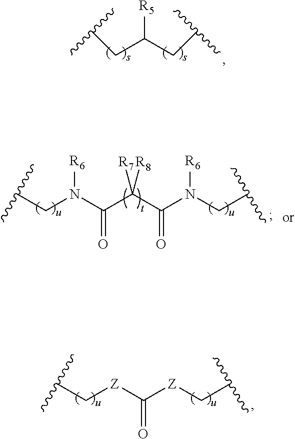

wherein:
   R₅ is OH, SH, or NR₁₀R₁₁;
   each R₆ is independently H, C₁-C₃ branched or unbranched alkyl, C₂-C₃ branched or unbranched alkenyl, or cycloalkyl;
   each R₇ and each R₈ is independently H, C₁-C₃ branched or unbranched alkyl, C₂-C₃ branched or unbranched alkenyl, halogen, OH, SH, or NR₁₀R₁₁, wherein each R₁₀ and R₁₁ is independently H, C₁-C₃ alkyl, or R₁₀ and R₁₁ are taken together to form a heterocyclic ring; R₇ and R₈ are taken together to form a ring;
   each s is independently 1, 2, 3, 4, or 5;
   each u is independently 1, 2, 3, 4, or 5;
   t is 1, 2, 3, 4 or 5;
   each Z is independently absent, O, S, or NR₁₂, wherein R₁₂ is H, C₁-C₇ branched or unbranched alkyl, or C₂-C₇ branched or unbranched alkenyl, and
   Q is O, S, or NR₁₃, wherein each R₁₃ is H, or C₁-C₅ alkyl.

In some embodiments, B is C₃-C₂₀ alkyl.

In some embodiments, W in formula (I) may alternatively be

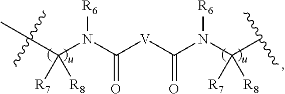

wherein:
V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups;
each R₆ is independently H, C₁-C₃ branched or unbranched alkyl, C₂-C₃ branched or unbranched alkenyl, or cycloalkyl;
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein each R₁₀ and R₁₁ is independently H, C₁-C₃ alkyl, or R₁₀ and R₁₁ are taken together to form a heterocyclic ring;
each v is independently 0, 1, 2, 3, 4, or 5;
R₁₇ is OH, SH, or N(CH₃)₂; and
each u is independently 1, 2, 3, 4, or 5.

In some embodiments, W in formula (I) may alternatively be

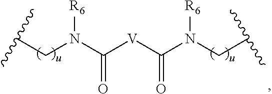

wherein:
V is C₂-C₁₀ alkenylene, C₂-C₁₀ alkynylene, or C₂-C₁₀ heteroalkylene;
each R₆ is independently H, C₁-C₃ branched or unbranched alkyl, C₂-C₃ branched or unbranched alkenyl, or cycloalkyl; and
each u is independently 1, 2, 3, 4, or 5.

In some embodiments, W in formula (I) may alternatively be

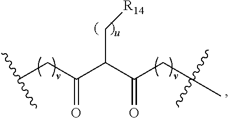

wherein:
R₁₄ is a heterocyclic;
each v is independently 0, 1, 2, 3, 4, or 5; and
each u is independently 1, 2, 3, 4, or 5.

In some embodiments, W in formula (I) may alternatively be

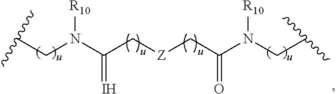

wherein:
Z is O, S, —C((CH₂)ᵥN(R₁₅)₂)—, or N(R₁₅), wherein R₁₅ is H, C₁-C₄ branched or unbranched alkyl, and v is 0, 1, 2, 3, 4, or 5;
each R₁₀ is independently H, or C₁-C₃ alkyl; and
each u is independently 0, 1, 2, 3, 4, or 5.

In some embodiments, W in formula (I) may alternatively be

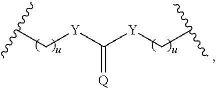

wherein:
each Y is a divalent heterocyclic;
Q is O, S, or NH; and
each u is independently 1, 2, 3, 4, or 5.

In some embodiments, W in formula (I) may alternatively be

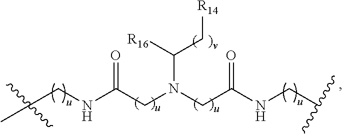

wherein:
R₁₄ is a heterocyclic, NR₁₀R₁₁, C(O)NR₁₀R₁₁, or C(S)NR₁₀R₁₁, wherein each R₁₀ and R₁₁ is independently H, C₁-C₃ alkyl, C₃-C₇ cycloalkyl, C₃-C₇ cycloalkenyl, optionally substituted with one or more NH and/or oxo groups, or R₁₀ and R₁₁ are taken together to form a heterocyclic ring;
R₁₆ is H, ═O, ═S, or CN;
each v is independently 0, 1, 2, 3, 4, or 5; and
each u is independently 1, 2, 3, 4, or 5.

In some embodiments, W in formula (I) may alternatively be

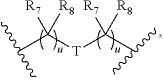

wherein:
T is —NHC(O)O—, —OC(O)NH—, or a divalent heterocyclic optionally substituted with one or more —(CH₂)ᵥOH, —(CH₂)ᵥSH, and/or —(CH₂)ᵥ-halogen groups;
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein each R₁₀ and R₁₁ is independently H, C₁-C₃ alkyl, or R₁₀ and R₁₁ are taken together to form a heterocyclic ring;
R₁₇ is OH, SH, or N(CH₃)₂;
each v is independently 0, 1, 2, 3, 4, or 5; and
each u is independently 1, 2, 3, 4, or 5.

In some embodiments, W in formula (I) may alternatively be

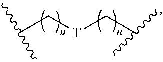

wherein:
T is —NHC(O)O—, —OC(O)NH—, or a divalent heterocyclic; and
each u is independently 1, 2, 3, 4, or 5.

In some embodiments, when Z is not absent, the adjacent R₁ and R₂ cannot be OH, NR₁₀R₁₁, or SH.

In some embodiments, the heterocyclic is a piperazine, piperazine dione, piperazine-2,5-dione, piperidine, pyrrolidine, piperidinol, dioxopiperazine, bis-piperazine, aromatic or heteroaromatic.

In some embodiments, the ionizable lipid is represented by formula (IX):

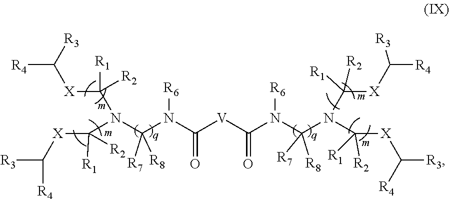

pharmaceutically acceptable salts thereof, and stereoisomers of any of the foregoing, wherein
each R₁ and each R₂ is independently H, C₁-C₃ branched or unbranched alkyl, OH, halogen, SH, or NR₁₀R₁₁, or
each R₁ and each R₂ are independently taken together with the carbon atom(s) to which they are attached to form a cyclic ring;
each R₁₀ and R₁₁ is independently H, C₁-C₃ branched or unbranched alkyl, or R₁₀ and R₁₁ are taken together to form a heterocyclic ring;
each R₃ and each R₄ is independently H, C₂-C₁₄ branched or unbranched alkyl (e.g., C₃-C₁₀ branched or unbranched alkyl), or C₃-C₁₀ branched or unbranched alkenyl, provided that at least one of R₃ and R₄ is not H;
each X is independently a biodegradable moiety;
each q is independently 2, 3, 4, or 5;
V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups;
each R₆ is independently H, C₁-C₃ branched or unbranched alkyl, C₂-C₃ branched or unbranched alkenyl, or cycloalkyl;
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein each v is independently 0, 1, 2, 3, 4, or 5, and R₁₇ is OH, SH, or N(CH₃)₂; and
each m is independently 1, 2, 3, 4, 5, 6, 7, 8, 9 or 10.

In some embodiments, V is a branched or unbranched C₂-C₃ alkylene. In some embodiments, V is a C₂-C₃ alkylene substituted with OH. In some embodiments, V is a branched or unbranched C₂-C₃ alkenylene. In some embodiments, each R₆ is independently H or methyl.

In some embodiments, the ionizable lipid is represented by one of the following formulas

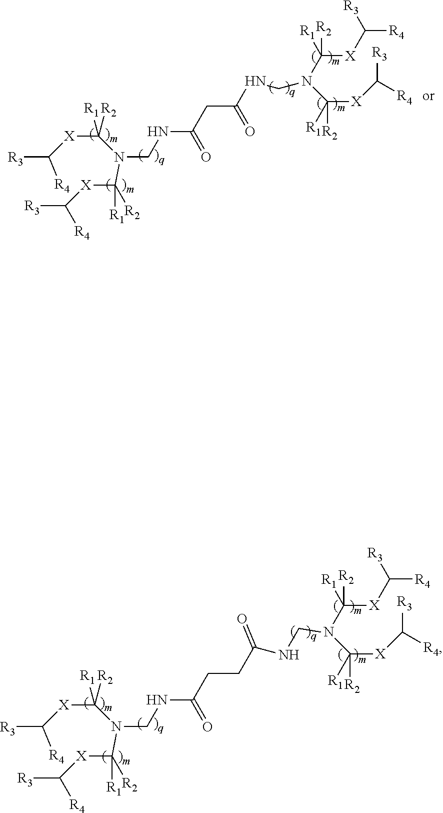

wherein the definition for the variables are the same as those in formula (X).

In some embodiments, the disclosure relates to ionizable lipids of Formula (XI):

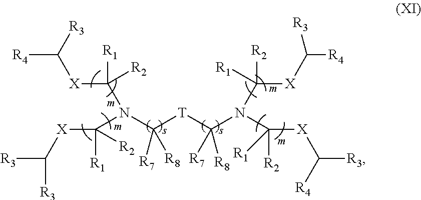

pharmaceutically acceptable salts thereof, and stereoisomers of any of the foregoing, wherein
each R₁ and each R₂ is independently H, C₁-C₃ branched or unbranched alkyl, OH, halogen, SH, or NR₁₀R₁₁, or
each R₁ and each R₂ are independently taken together with the carbon atom(s) to which they are attached to form a cyclic ring;
each R₁₀ and R₁₁ is independently H, C₁-C₃ branched or unbranched alkyl, or R₁₀ and R₁₁ are taken together to form a heterocyclic ring;
each R₃ and each R₄ is independently H, C₂-C₁₄ branched or unbranched alkyl (e.g., C₃-C₁₀ branched or unbranched alkyl), or C₃-C₁₀ branched or unbranched alkenyl, provided that at least one of R₃ and R₄ is not H;
each X is independently a biodegradable moiety;
each s is independently 1, 2, 3, 4, or 5;
T is —NHC(O)O—, —OC(O)NH—, or a divalent heterocyclic optionally substituted with one or more —(CH₂)ᵥOH, —(CH₂)ᵥSH, —(CH₂)ᵥ-halogen groups, each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein R₁₇ is OH, SH, or N(CH₃)₂;
each v is independently 0, 1, 2, 3, 4, or 5; and
each m is independently 1, 2, 3, 4, 5, 6, 7, 8, 9 or 10.

In some embodiments, T is a divalent heterocyclic (e.g., a divalent piperazine, or a divalent dioxopiperazine) optionally substituted with —(CH₂)ᵥOH, wherein v is independently 0, 1, or 2.

In some embodiments, in each of the above formulas, X is —OC(O)—, —C(O)O—, —SS—, —N(R¹⁸)C(O)—, —C(O)N(R¹⁸)—, —C(O—R₁₃)—O—, —C(O)O(CH₂)ₐ—, —OC(O)(CH₂)ₐ—, —C(O)N(R¹⁸)(CH₂)ₐ—, —N(R¹⁸)C(O)(CH₂)ₐ—, —C(O—R₁₃)—O—(CH₂)ₐ—, wherein each R¹⁸ is independently H, alkyl, alkenyl, cycloalkyl, hydroxyalkyl, or aminoalkyl, each R₁₃ is independently C₃-C₁₀ alkyl, and each a is independently 0-16. In one embodiment, each X is independently —OCO—, —COO—, —NHCO—, or —CONH—. In one embodiment, at least one X is —SS—.

More embodiments of the ionizable lipid of formula (I), in the Ionizable lipid compounds group i), may be found in PCT Application No. PCT/US22/50725, filed on Nov. 22, 2022, the content of which is incorporated herein by reference in its entirety. In particular, all the ionizable lipids of formulas (I)—(XII) of PCT Application No. PCT/US22/50725 are suitable for use as the ionizable lipids in this disclosure, and are incorporated herein by reference in its entirety.

Certain exemplary ionizable lipid compounds disclosed herein are set forth in Table I below.

TABLE I
Exemplary ionizable lipid compounds.

| Lipid | | |
|---|---|---|
| No. | Structure | Name |
| 2303 | 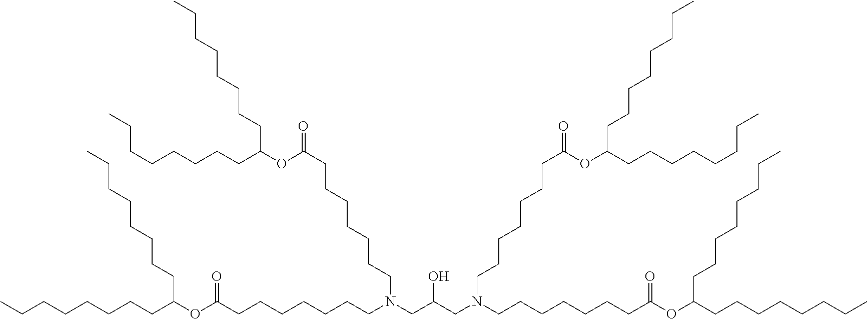 [SMILES: CCCCCCCCC(CCCCCCCC)OC(=O)CCCCCCCN(CCCCCCCC(=O)OC(CCCCCCCC)CCCCCCCC)CC(O)CN(CCCCCCCC(=O)OC(CCCCCCCC)CCCCCCCC)CCCCCCCC(=O)OC(CCCCCCCC)CCCCCCCC] | heptadecan-9-yl 8-[(3- {bis[8-(heptadecan-9- yloxy)-8- oxooctyl]amino}-2- hydroxypropyl)[8- (heptadecan-9-yloxy)-8- oxooctyl]amino] octanoate |
| 2302 | 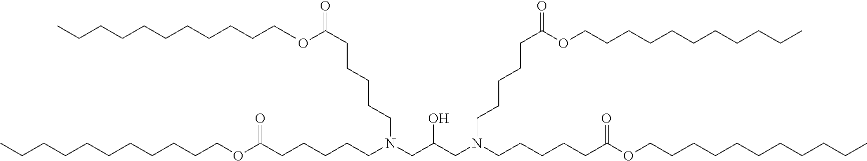 | undecyl 6-[(3-{bis[6- oxo-6- (undecyloxy)hexyl] amino}-2- hydroxypropyl)[6- oxo-6- (undecyloxy)hexyl] amino]hexanoate |
| 2299 | 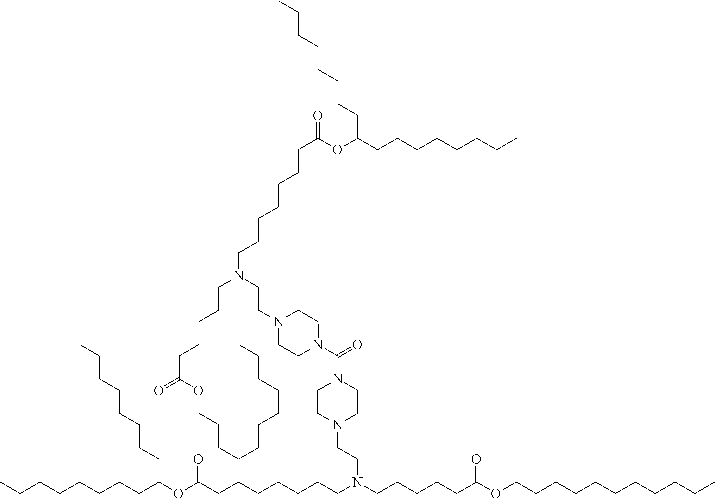 [SMILES: CCCCCCCCCCCOC(=O)CCCCCN(CCCCCCCC(=O)OC(CCCCCCCC)CCCCCCCC)CCN1CCN(C(=O)N2CCN(CCN(CCCCCCCC(=O)OC(CCCCCCCC)CCCCCCCC)CCCCCC(=O)OCCCCCCCCCCC)CC2)CC1] | heptadecan-9-yl 8-[(2- {4-[4-(2-{[8- (heptadecan-9-yloxy)-8- oxooctyl][6-oxo-6- (undecyloxy)hexyl] amino}ethyl) piperazine-1- carbonyl]piperazin-1- yl}ethyl)[6-oxo-6- (undecyloxy)hexyl] amino]octanoate |
| 2289 | 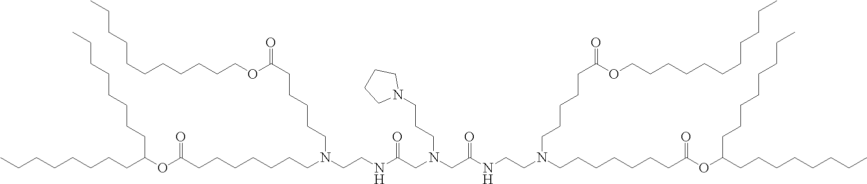 [SMILES: CCCCCCCCCCCOC(=O)CCCCCN(CCCCCCCC(=O)OC(CCCCCCCC)CCCCCCCC)CCNC(=O)CN(CCCN1CCCC1)CC(=O)NCCN(CCCCCCCC(=O)OC(CCCCCCCC)CCCCCCCC)CCCCCC(=O)OCCCCCCCCCCC] | heptadecan-9-yl 8-({2- [2-({[(2-{[8- (heptadecan-9-yloxy)-8- oxooctyl][6-oxo-6- (undecyloxy)hexyl] amino}ethyl)carbamoyl] methyl}[3-(pyrrolidin-1- yl)propyl]amino) acetamido]ethyl}[6- oxo-6- |
| 2288 | 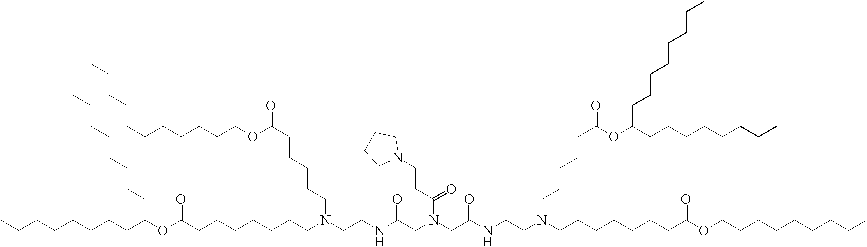 [SMILES: CCCCCCCCCCCOC(=O)CCCCCN(CCCCCCCC(=O)OC(CCCCCCCC)CCCCCCCC)CCNC(=O)CN(CC(=O)NCCN(CCCCCCCC(=O)OCCCCCCCCC)CCCCCC(=O)OC(CCCCCCCC)CCCCCCCC)C(=O)CCN1CCCC1] | heptadecan-9-yl 8-({2- [2-(N-{[(2-{[8- (heptadecan-9-yloxy)-8- oxooctyl][6-oxo-6- (undecyloxy)hexyl] amino}ethyl) carbamoyl]methyl}-3- (pyrrolidin-1- yl)propanamido) acetamido]ethyl}[6- oxo-6- |
| 2286 | 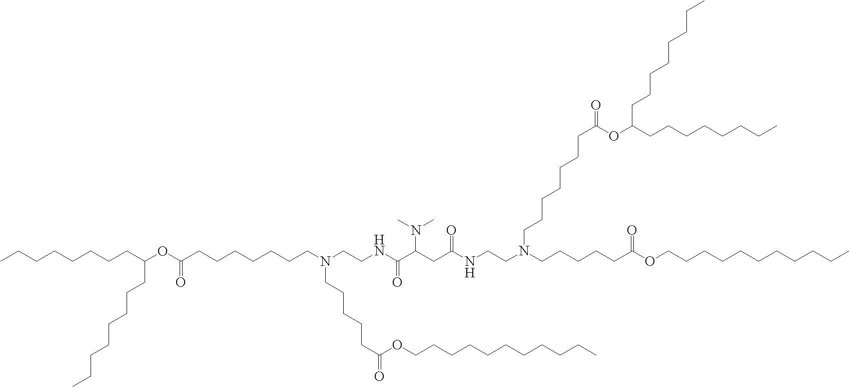 [SMILES: CCCCCCCCCCCOC(=O)CCCCCN(CCCCCCCC(=O)OC(CCCCCCCC)CCCCCCCC)CCNC(=O)CC(C(=O)NCCN(CCCCCCCC(=O)OC(CCCCCCCC)CCCCCCCC)CCCCCC(=O)OCCCCCCCCCCC)N(C)C] | heptadecan-9-yl 8-({2- [2-(dimethylamino)-3- [(2-{[8-(heptadecan-9- yloxy)-8-oxooctyl][6- oxo-6- (undecyloxy)hexyl] amino}ethyl)carbamoyl] propanamido]ethyl} [6-oxo-6- (undecyloxy)hexyl] amino)octanoate |
| 2285 | 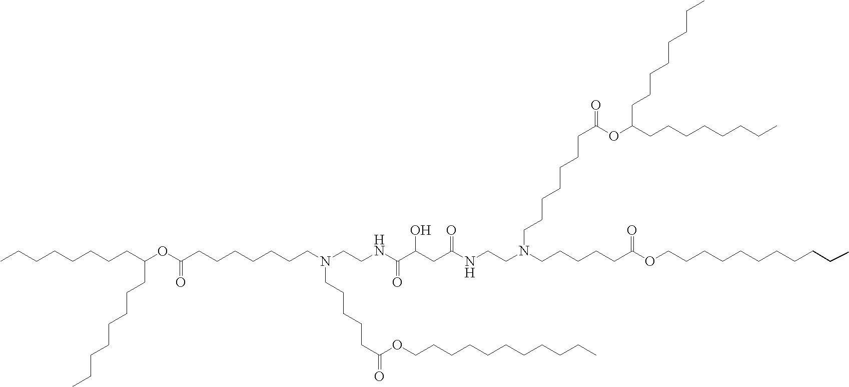 [SMILES: CCCCCCCCCCCOC(=O)CCCCCN(CCCCCCCC(=O)OC(CCCCCCCC)CCCCCCCC)CCNC(=O)CC(O)C(=O)NCCN(CCCCCCCC(=O)OC(CCCCCCCC)CCCCCCCC)CCCCCC(=O)OCCCCCCCCCCC] | heptadecan-9-yl 8-[(2- {3-[(2-{[8-(heptadecan- 9-yloxy)-8-oxooctyl][6- oxo-6- (undecyloxy)hexyl] amino}ethyl)carbamoyl]- 2-hydroxypropanamido} ethyl)[6-oxo-6- (undecyloxy)hexyl] amino]octanoate |
| 2284 | 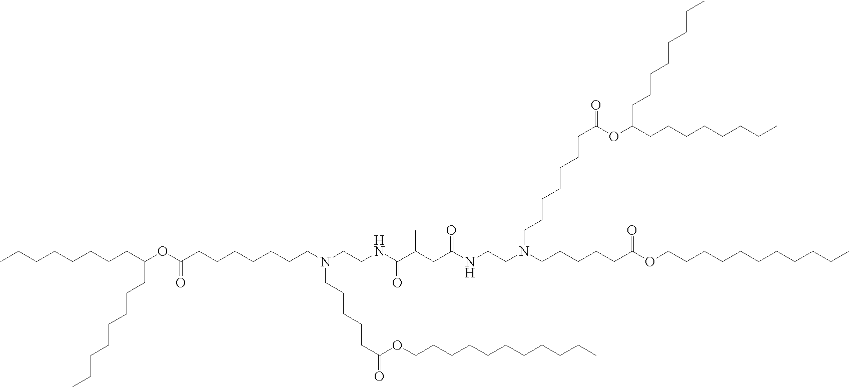 [SMILES: CCCCCCCCCCCOC(=O)CCCCCN(CCCCCCCC(=O)OC(CCCCCCCC)CCCCCCCC)CCNC(=O)CC(C)C(=O)NCCN(CCCCCCCC(=O)OC(CCCCCCCC)CCCCCCCC)CCCCCC(=O)OCCCCCCCCCCC] | heptadecan-9-yl 8-[(2- {3-[(2-{[8-(heptadecan- 9-yloxy)-8-oxooctyl][6- oxo-6- (undecyloxy)hexyl] amino}ethyl)carbamoyl]- 2-methylpropanamido} ethyl)[6-oxo-6- (undecyloxy)hexyl] amino]octanoate |
| 2283 | 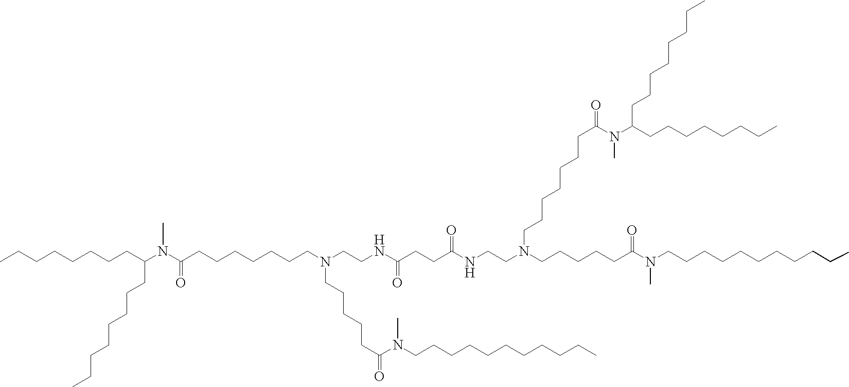 [SMILES: CCCCCCCCCCCN(C)C(=O)CCCCCN(CCCCCCCC(=O)N(C)C(CCCCCCCC)CCCCCCCC)CCNC(=O)CCC(=O)NCCN(CCCCCCCC(=O)N(C)C(CCCCCCCC)CCCCCCCC)CCCCCC(=O)N(C)CCCCCCCCCCC] | N,N′-bis[2-({7- [(heptadecan-9- yl)(methyl)carbamoyl] heptyl}{{5- [methyl(undecyl) carbamoyl]pentyl}) amino)ethyl] butanediamide |
| 2282 | 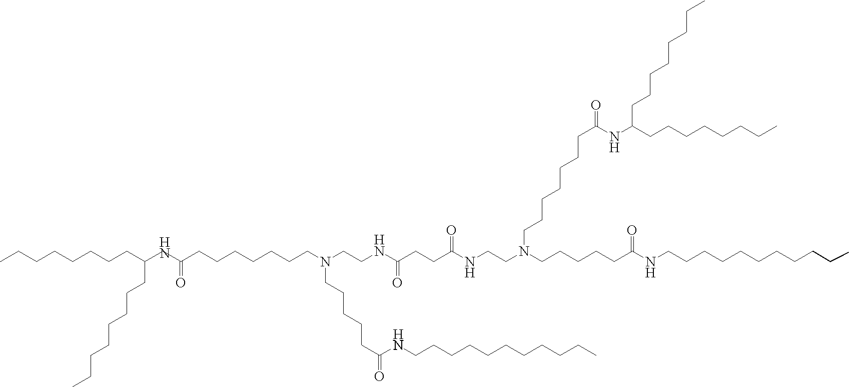 | N,N′-bis[2-({7- [(heptadecan-9- yl)carbamoyl]heptyl}[5- (undecylcarbamoyl) pentyl]amino)ethyl] butanediamide |
| 2281 | 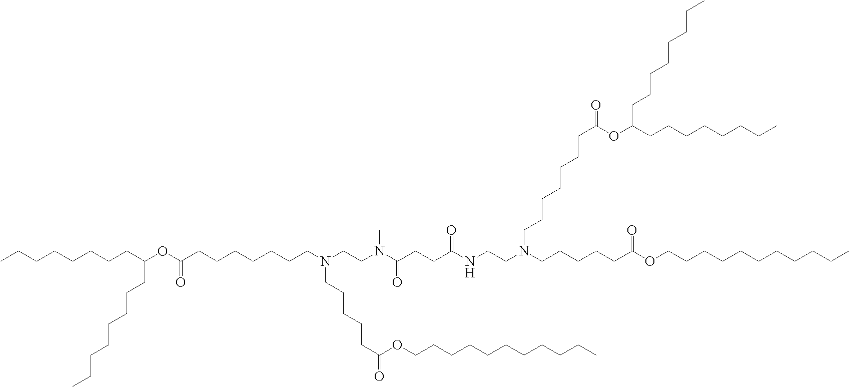 [SMILES: CCCCCCCCCCCOC(=O)CCCCCN(CCCCCCCC(=O)OC(CCCCCCCC)CCCCCCCC)CCNC(=O)CCC(=O)N(C)CCN(CCCCCCCC(=O)OC(CCCCCCCC)CCCCCCCC)CCCCCC(=O)OCCCCCCCCCCC] | heptadecan-9-yl 8-[(2- {3-[(2-{[8-(heptadecan- 9-yloxy)-8-oxooctyl][6- oxo-6- (undecyloxy)hexyl] amino}ethyl) carbamoyl]-N- methylpropanamido} ethyl)[6-oxo-6- (undecyloxy)hexyl] amino]octanoate |
| 2280 | 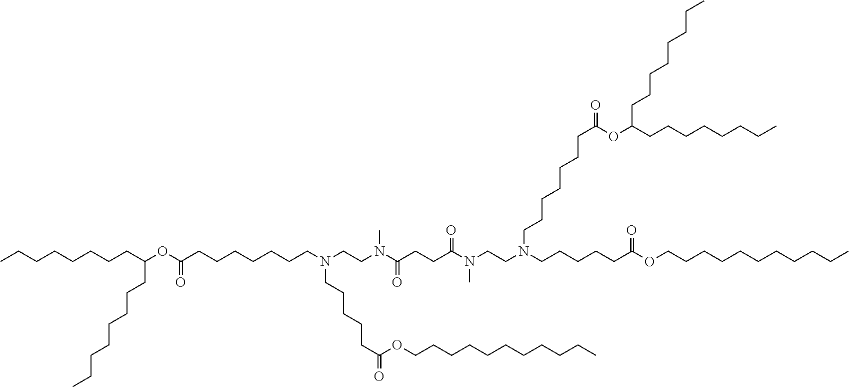 [SMILES: CCCCCCCCCCCOC(=O)CCCCCN(CCCCCCCC(=O)OC(CCCCCCCC)CCCCCCCC)CCN(C)C(=O)CCC(=O)N(C)CCN(CCCCCCCC(=O)OC(CCCCCCCC)CCCCCCCC)CCCCCC(=O)OCCCCCCCCCCC] | heptadecan-9-yl 8-[(2- {3-[(2-{[8-(heptadecan- 9-yloxy)-8-oxooctyl][6- oxo-6- (undecyloxy)hexyl] amino}ethyl)(methyl) carbamoyl]-N- methylpropanamido} ethyl)[6-oxo-6- (undecyloxy)hexyl] amino]octanoate |
| 2279 | 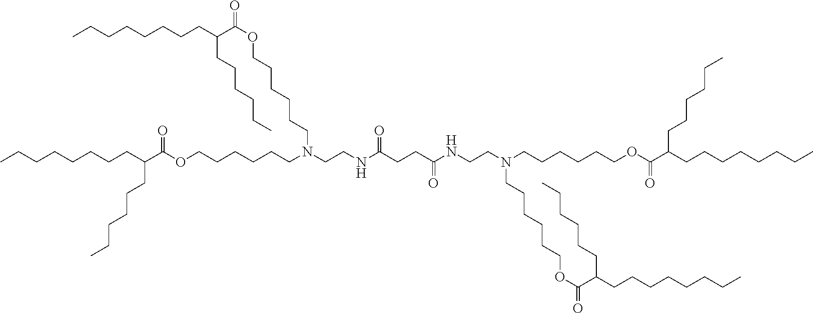 [SMILES: CCCCCCCCC(CCCCCC)C(=O)OCCCCCCN(CCCCCCOC(=O)C(CCCCCC)CCCCCCCC)CCNC(=O)CCC(=O)NCCN(CCCCCCOC(=O)C(CCCCCC)CCCCCCCC)CCCCCCOC(=O)C(CCCCCC)CCCCCCCC] | 6-({2-[3-({2-[bis({6-[(2- hexyldecanoyl)oxy] hexyl})amino]ethyl} carbamoyl)propanamido] ethyl}{{6-[(2- hexyldecanoyl)oxy] hexyl})amino)hexyl 2- hexyldecanoate |
| 2278 | 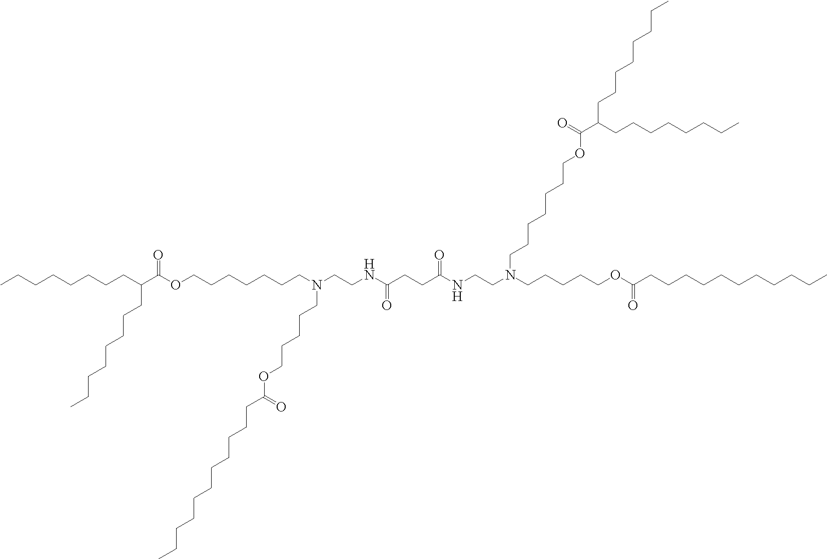 [SMILES: CCCCCCCCCCCC(=O)OCCCCCN(CCCCCCCOC(=O)C(CCCCCCCC)CCCCCCCC)CCNC(=O)CCC(=O)NCCN(CCCCCCCOC(=O)C(CCCCCCCC)CCCCCCCC)CCCCCOC(=O)CCCCCCCCCCC] | 5-[(2-{3-[(2-{[5- (dodecanoyloxy) pentyl]({7-[(2- octyldecanoyl)oxy] heptyl})amino}ethyl) carbamoyl]propanamido} ethyl)({7-[(2- octyldecanoyl)oxy] heptyl})amino]pentyl dodecanoate |
| 2275 | 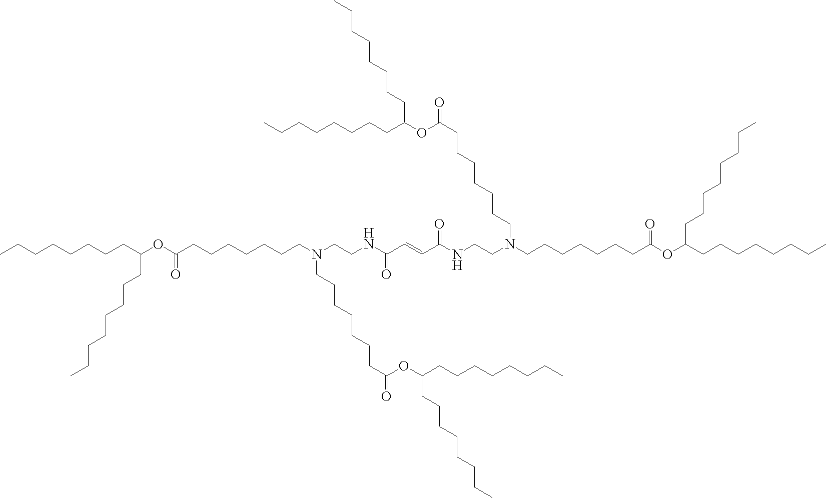 [SMILES: CCCCCCCCC(CCCCCCCC)OC(=O)CCCCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCCCCC(=O)OC(CCCCCCCC)CCCCCCCC] | heptadecan-9-yl 8-[(2- {3-[(2-{bis[8- (heptadecan-9-yloxy)-8- oxooctyl]amino}ethyl) carbamoyl]propanamido} ethyl)[8-(heptadecan-9- yloxy)-8- oxooctyl]amino] octanoate |
| 2274 | 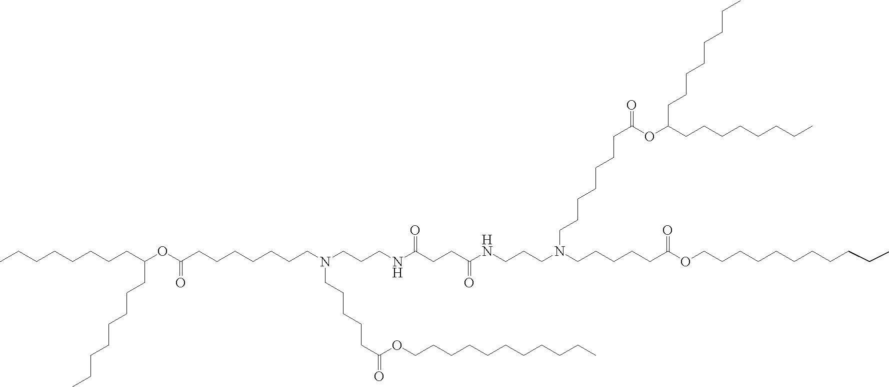 [SMILES: CCCCCCCCCCCOC(=O)CCCCCN(CCCCCCCC(=O)OC(CCCCCCCC)CCCCCCCC)CCCNC(=O)CCC(=O)NCCCN(CCCCCCCC(=O)OC(CCCCCCCC)CCCCCCCC)CCCCCC(=O)OCCCCCCCCCCC] | heptadecan-9-yl 8-[(3- {3-[(3-{[8-(heptadecan- 9-yloxy)-8-oxooctyl][6- oxo-6- (undecyloxy)hexyl] amino}propyl) carbamoyl]propanamido} propyl)[6-oxo-6- (undecyloxy)hexyl] amino]octanoate |
| 2273 | 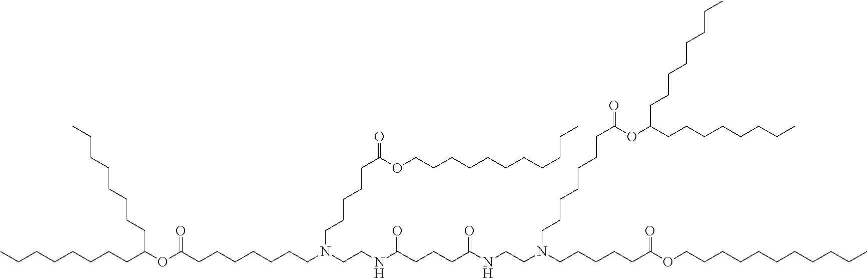 [SMILES: CCCCCCCCCCCOC(=O)CCCCCN(CCCCCCCC(=O)OC(CCCCCCCC)CCCCCCCC)CCNC(=O)CCCC(=O)NCCN(CCCCCCCC(=O)OC(CCCCCCCC)CCCCCCCC)CCCCCC(=O)OCCCCCCCCCCC] | heptadecan-9-yl 8-[(2- {4-[(2-{[8-(heptadecan- 9-yloxy)-8-oxooctyl][6- oxo-6- (undecyloxy)hexyl] amino}ethyl)carbamoyl] butanamido}ethyl) [6-oxo-6- (undecyloxy)hexyl] aminoloctanoate |
| 2272 | 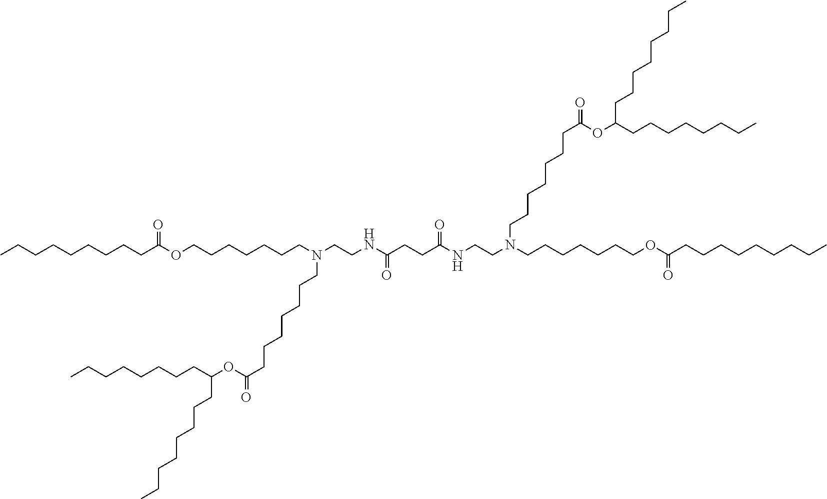 [SMILES: CCCCCCCCCC(=O)OCCCCCCCN(CCCCCCCC(=O)OC(CCCCCCCC)CCCCCCCC)CCNC(=O)CCC(=O)NCCN(CCCCCCCOC(=O)CCCCCCCCC)CCCCCCCC(=O)OC(CCCCCCCC)CCCCCCCC] | 7-[(2-{3-[(2-{[7- (decanoyloxy)heptyl][8- (heptadecan-9-yloxy)-8- oxooctyl]amino}ethyl) carbamoyl] propanamido}ethyl) [8-(heptadecan-9- yloxy)-8- oxooctyl]amino]heptyl decanoate |
| 2271 | 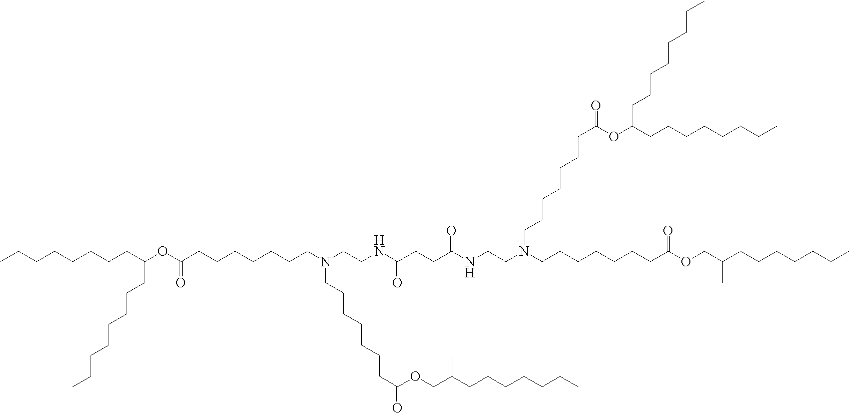 [SMILES: CCCCCCCCC(CCCCCCCC)OC(=O)CCCCCCCN(CCCCCCCC(=O)OCC(C)CCCCCCC)CCNC(=O)CCC(=O)NCCN(CCCCCCCC(=O)OCC(C)CCCCCCC)CCCCCCCC(=O)OC(CCCCCCCC)CCCCCCCC] | heptadecan-9-yl 8-[(2- {3-[(2-{[8-(heptadecan- 9-yloxy)-8-oxooctyl]({8- [(2-methylnonyl)oxy]-8- oxooctyl})amino}ethyl) carbamoyl]propanamido} ethyl) ({8-[(2- methylnonyl)oxy]-8- oxooctyl})amino] octanoate |
| 2253 | 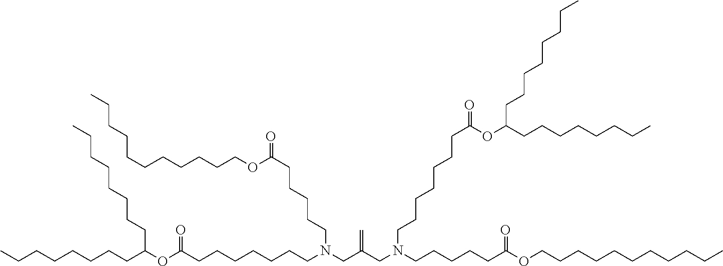 [SMILES: C=C(CN(CCCCCCCC(=O)OC(CCCCCCCC)CCCCCCCC)CCCCCC(=O)OCCCCCCCCCCC)CN(CCCCCCCC(=O)OC(CCCCCCCC)CCCCCCCC)CCCCCC(=O)OCCCCCCCCCCC] | heptadecan-9-yl 8-{[2- ({[8-(heptadecan-9- yloxy)-8-oxooctyl][6- oxo-6- (undecyloxy)hexyl] amino}methyl)prop- 2-en-1-yl][6-oxo-6- (undecyloxy)hexyl] amino}octanoate |
| 2252 | 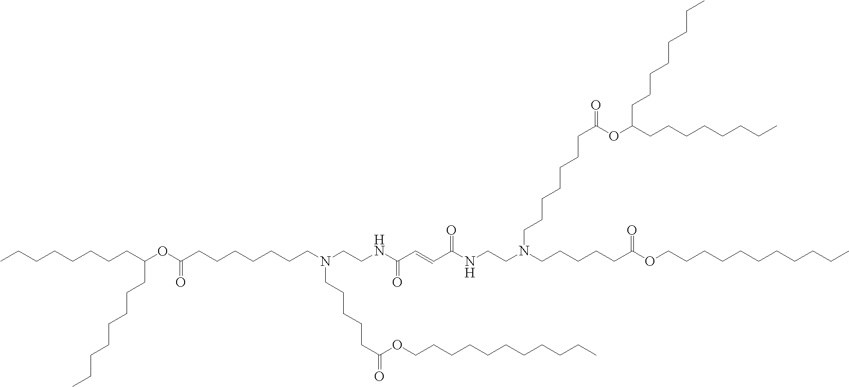 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCCC(=O)OCCCCCCCCCCC] | heptadecan-9-yl 8-({2- [(2E)-3-[(2-{[8- (heptadecan-9-yloxy)-8- oxooctyl][6-oxo-6- (undecyloxy)hexyl] amino}ethyl) carbamoyl]prop- 2-enamido]ethyl} [6-oxo-6- (undecyloxy)hexyl] amino)octanoate |
| 2251 | 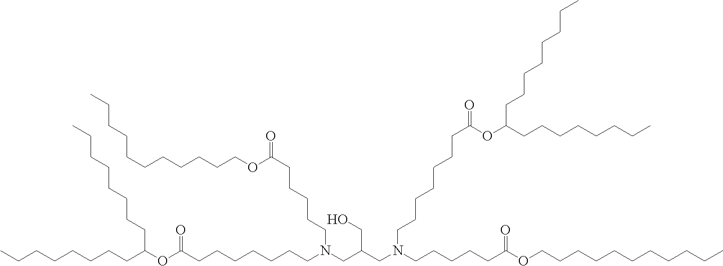 [SMILES: CCCCCCCCCCCOC(=O)CCCCCN(CCCCCCCC(=O)OC(CCCCCCCC)CCCCCCCC)CC(CO)CN(CCCCCCCC(=O)OC(CCCCCCCC)CCCCCCCC)CCCCCC(=O)OCCCCCCCCCCC] | heptadecan-9-yl 8-{[2- ({[8-(heptadecan-9- yloxy)-8-oxooctyl][6- oxo-6- (undecyloxy)hexyl] amino}methyl)-3- hydroxypropyl][6-oxo- 6-(undecyloxy)hexyl] amino}octanoate |
| 2247 | 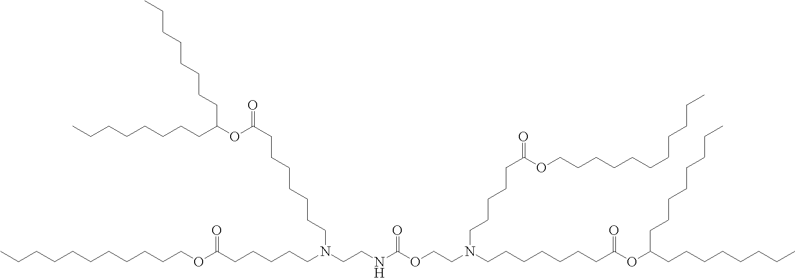 [SMILES: CCCCCCCCCCCOC(=O)CCCCCN(CCCCCCCC(=O)OC(CCCCCCCC)CCCCCCCC)CCNC(=O)OCCN(CCCCCCCC(=O)OC(CCCCCCCC)CCCCCCCC)CCCCCC(=O)OCCCCCCCCCCC] | heptadecan-9-yl 8-[(2- {[(2-{[8-(heptadecan-9- yloxy)-8-oxooctyl][6- oxo-6- (undecyloxy)hexyl] amino}ethoxy) carbonyl]amino} ethyl)[6-oxo-6- (undecyloxy)hexyl] amino]octanoate |
| 2246 | 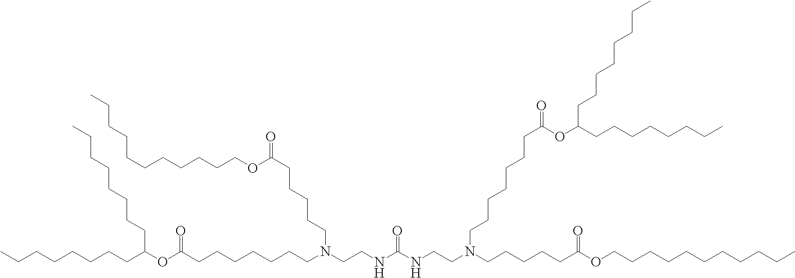 [SMILES: CCCCCCCCCCCOC(=O)CCCCCN(CCCCCCCC(=O)OC(CCCCCCCC)CCCCCCCC)CCNC(=O)NCCN(CCCCCCCC(=O)OC(CCCCCCCC)CCCCCCCC)CCCCCC(=O)OCCCCCCCCCCC] | heptadecan-9-yl 8-[(2- {[(2-{[8-(heptadecan-9- yloxy)-8-oxooctyl][6- oxo-6- (undecyloxy)hexyl] amino}ethyl) carbamoyl]amino} ethyl)[6-oxo-6- (undecyloxy)hexyl] amino]octanoate |
| 2221 | 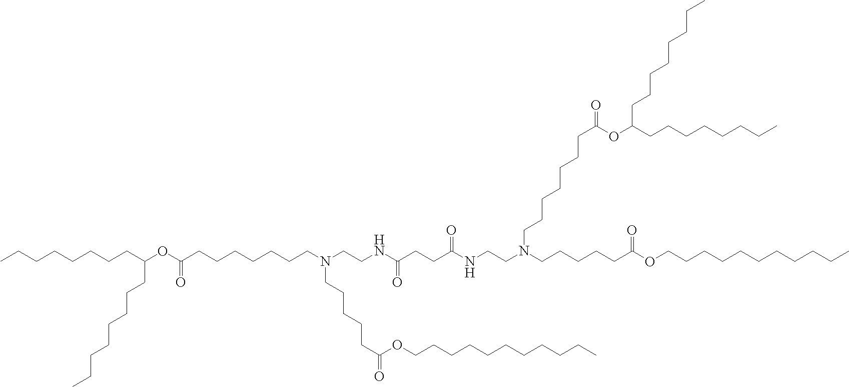 [SMILES: CCCCCCCCCCCOC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCC(=O)OCCCCCCCCCCC] | heptadecan-9-yl 8-[(2- {3-[(2-{[8-(heptadecan- 9-yloxy)-8-oxooctyl][6- oxo-6- (undecyloxy)hexyl] amino}ethyl) carbamoyl]prop anamido}ethyl)[6-oxo-6- (undecyloxy)hexyl] amino]octanoate |
| 2220 | 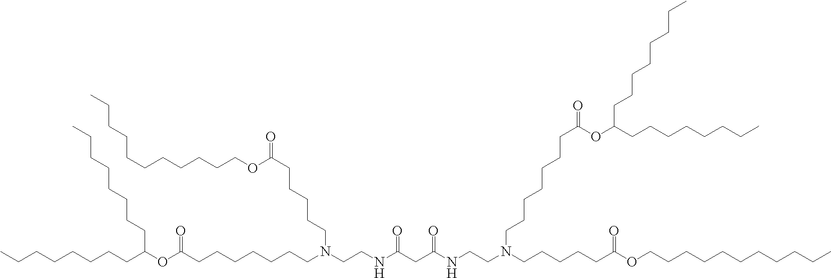 [SMILES: CCCCCCCCCCCOC(=O)CCCCCN(CCCCCCCC(=O)OC(CCCCCCCC)CCCCCCCC)CCNC(=O)CC(=O)NCCN(CCCCCCCC(=O)OC(CCCCCCCC)CCCCCCCC)CCCCCC(=O)OCCCCCCCCCCC] | heptadecan-9-yl 8-[(2- {2-[(2-{[8-(heptadecan- 9-yloxy)-8-oxooctyl][6- oxo-6- (undecyloxy)hexyl] amino}ethyl) carbamoyl]acetamido} ethyl)[6-oxo-6- (undecyloxy)hexyl] aminoloctanoate |
| 2219 | 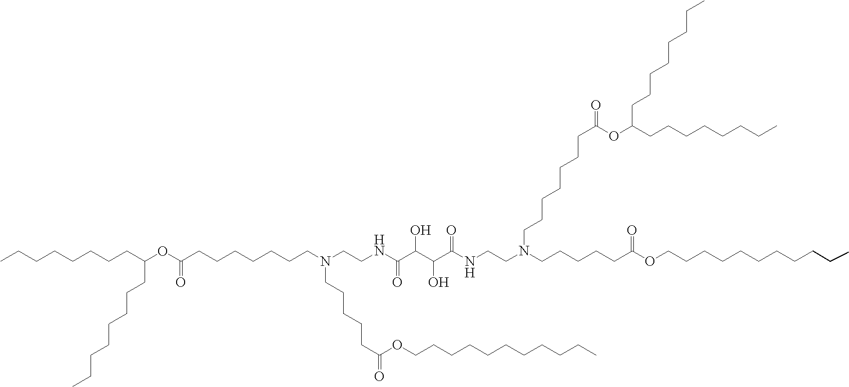 [SMILES: CCCCCCCCCCCOC(=O)CCCCCN(CCCCCCCC(=O)OC(CCCCCCCC)CCCCCCCC)CCNC(=O)C(O)C(O)C(=O)NCCN(CCCCCCCC(=O)OC(CCCCCCCC)CCCCCCCC)CCCCCC(=O)OCCCCCCCCCCC] | heptadecan-9-yl 8-[(2- {3-[(2-{[8-(heptadecan- 9-yloxy)-8-oxooctyl][6- oxo-6- (undecyloxy)hexyl] amino}ethyl) carbamoyl]-2,3- dihydroxypropanamido} ethyl)[6-oxo-6- (undecyloxy)hexyl] amino]octanoate |
| 2218 | 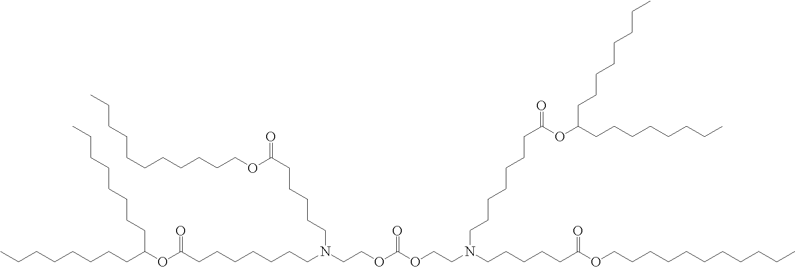 [SMILES: CCCCCCCCCCCOC(=O)CCCCCN(CCCCCCCC(=O)OC(CCCCCCCC)CCCCCCCC)CCOC(=O)OCCN(CCCCCCCC(=O)OC(CCCCCCCC)CCCCCCCC)CCCCCC(=O)OCCCCCCCCCCC] | heptadecan-9-yl 8-[(2- {[(2-{[8-(heptadecan-9- yloxy)-8-oxooctyl][6- oxo-6- (undecyloxy)hexyl] amino}ethoxy) carbonyl]oxy} ethyl)[6-oxo-6- (undecyloxy)hexyl] amino]octanoate |
| 2217 | 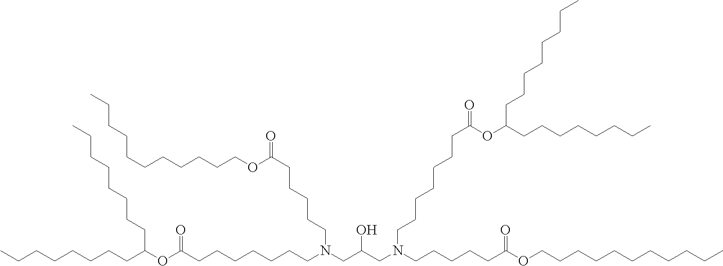 [SMILES: CCCCCCCCCCCOC(=O)CCCCCN(CCCCCCCC(=O)OC(CCCCCCCC)CCCCCCCC)CC(O)CN(CCCCCCCC(=O)OC(CCCCCCCC)CCCCCCCC)CCCCCC(=O)OCCCCCCCCCCC] | heptadecan-9-yl 8-[(3- {[8-(heptadecan-9- yloxy)-8-oxooctyl][6- oxo-6- (undecyloxy)hexyl] amino}-2-hydroxypropyl) [6-oxo-6- (undecyloxy)hexyl] amino]octanoate |
| 2213 | 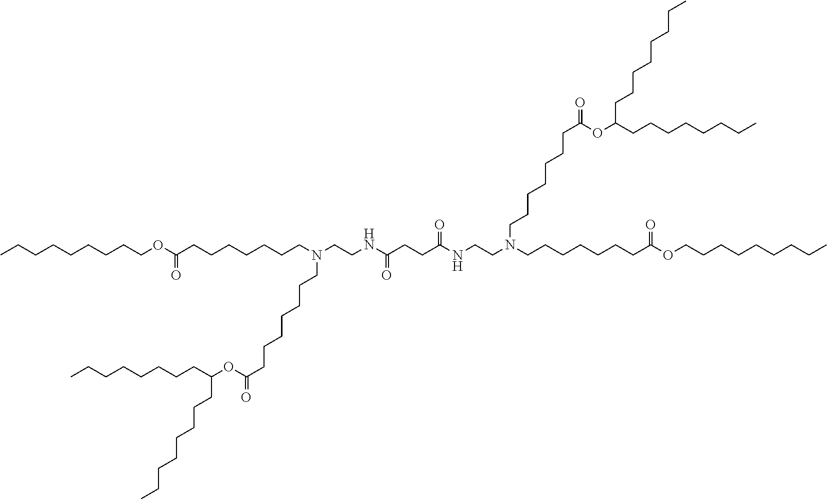 [SMILES: CCCCCCCCCOC(=O)CCCCCCCN(CCCCCCCC(=O)OC(CCCCCCCC)CCCCCCCC)CCNC(=O)CCC(=O)NCCN(CCCCCCCC(=O)OCCCCCCCCC)CCCCCCCC(=O)OC(CCCCCCCC)CCCCCCCC] | heptadecan-9-yl 8-[(2- {3-[(2-{[8-(heptadecan- 9-yloxy)-8-oxooctyl][8- (nonyloxy)-8- oxooctyl]amino}ethyl) carbamoyl] propanamido}ethyl)[8- (nonyloxy)-8- oxooctyl]amino] octanoate |
| 2212 | 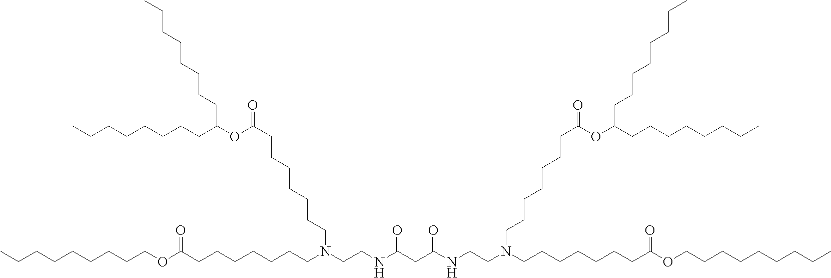 [SMILES: CCCCCCCCCOC(=O)CCCCCCCN(CCCCCCCC(=O)OC(CCCCCCCC)CCCCCCCC)CCNC(=O)CC(=O)NCCN(CCCCCCCC(=O)OCCCCCCCCC)CCCCCCCC(=O)OC(CCCCCCCC)CCCCCCCC] | heptadecan-9-yl 8-[(2- {2-[(2-{[8-(heptadecan- 9-yloxy)-8-oxooctyl][8- (nonyloxy)-8- oxooctyl]amino}ethyl) carbamoyl]acetamido} ethyl)[8-(nonyloxy)-8- oxooctyl]amino] octanoate |
| 2211 | 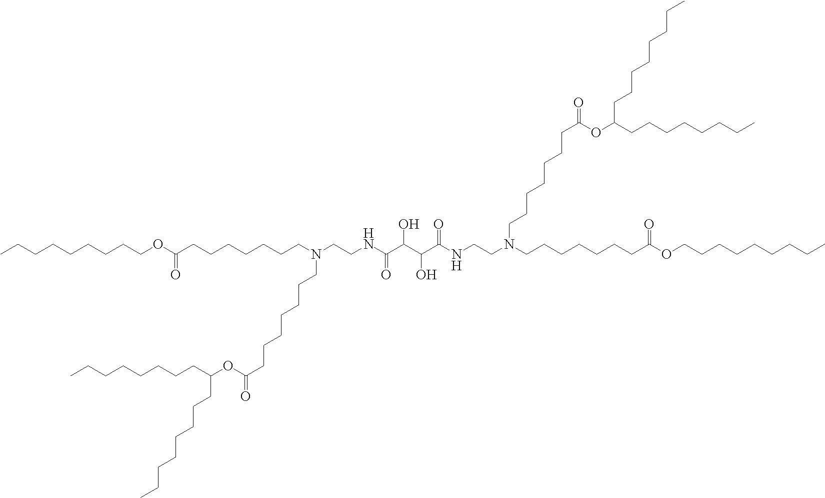 [SMILES: CCCCCCCCCOC(=O)CCCCCCCN(CCCCCCCC(=O)OC(CCCCCCCC)CCCCCCCC)CCNC(=O)C(O)C(O)C(=O)NCCN(CCCCCCCC(=O)OCCCCCCCCC)CCCCCCCC(=O)OC(CCCCCCCC)CCCCCCCC] | heptadecan-9-yl 8-[(2- {3-[(2-{[8-(heptadecan- 9-yloxy)-8-oxooctyl][8- (nonyloxy)-8- oxooctyl]amino}ethyl) carbamoyl]-2,3- dihydroxypropanamido} ethyl)[8-(nonyloxy)-8- oxooctyl]amino] octanoate |
| 2210 | 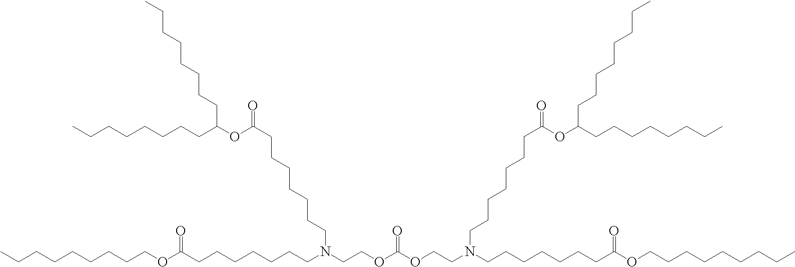 [SMILES: CCCCCCCCCOC(=O)CCCCCCCN(CCCCCCCC(=O)OC(CCCCCCCC)CCCCCCCC)CCOC(=O)OCCN(CCCCCCCC(=O)OCCCCCCCCC)CCCCCCCC(=O)OC(CCCCCCCC)CCCCCCCC] | heptadecan-9-yl 8-[(2- {[(2-{[8-(heptadecan-9- yloxy)-8-oxooctyl][8- (nonyloxy)-8- oxooctyl]amino}ethoxy) carbonyl]oxy}ethyl)[8- (nonyloxy)-8- oxooctyl]amino] octanoate |
| 2209 | 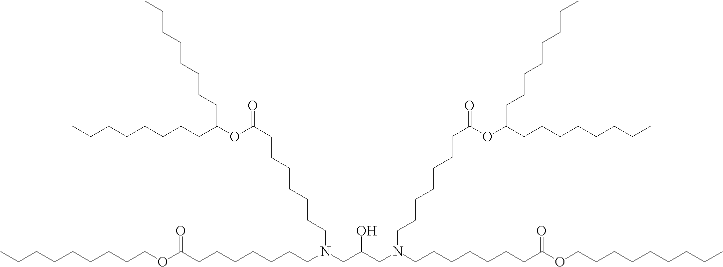 [SMILES: CCCCCCCCCOC(=O)CCCCCCCN(CCCCCCCC(=O)OC(CCCCCCCC)CCCCCCCC)CC(O)CN(CCCCCCCC(=O)OCCCCCCCCC)CCCCCCCC(=O)OC(CCCCCCCC)CCCCCCCC] | heptadecan-9-yl 8-[(3- {[8-(heptadecan-9- yloxy)-8-oxooctyl][8- (nonyloxy)-8- oxooctyl]amino}-2- hydroxypropyl)[8- (nonyloxy)-8- oxooctyl]amino] octanoate |
| 2513 | 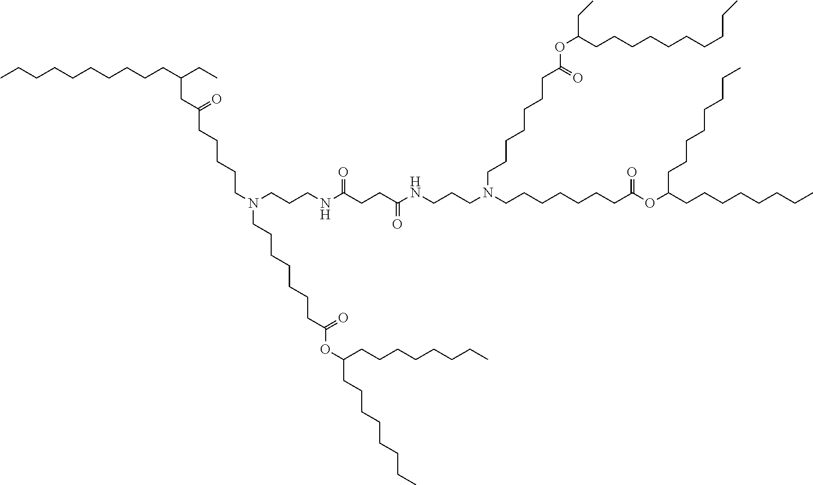 [SMILES: CCCCCCCCCCC(CC)CC(=O)CCCCCN(CCCCCCCC(=O)OC(CCCCCCCC)CCCCCCCC)CCCNC(=O)CCC(=O)NCCCN(CCCCCCCC(=O)OC(CC)CCCCCCCCCC)CCCCCCCC(=O)OC(CCCCCCCC)CCCCCCCC] | di(heptadecan-9-yl) 14,17-dioxo-9,22-bis(6- oxo-6-(tridecan-3- yloxy)hexyl)- 9,13,18,22- tetraazatriacontanedioate |
| | 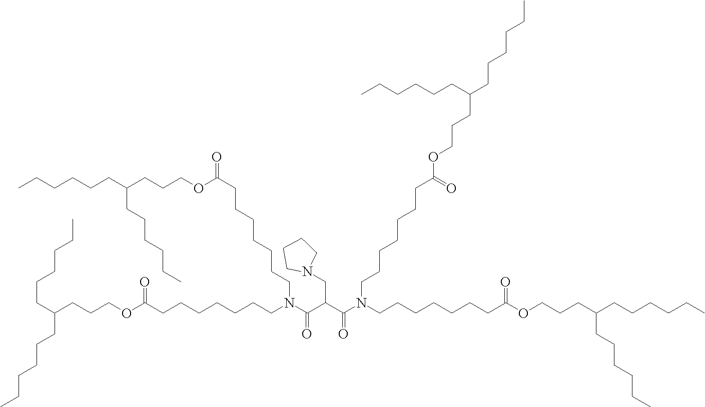 [SMILES: CCCCCCC(CCCCCC)CCCOC(=O)CCCCCCCN(CCCCCCCC(=O)OCCCC(CCCCCC)CCCCCC)C(=O)C(CN1CCCC1)C(=O)N(CCCCCCCC(=O)OCCCC(CCCCCC)CCCCCC)CCCCCCCC(=O)OCCCC(CCCCCC)CCCCCC] | |
| | 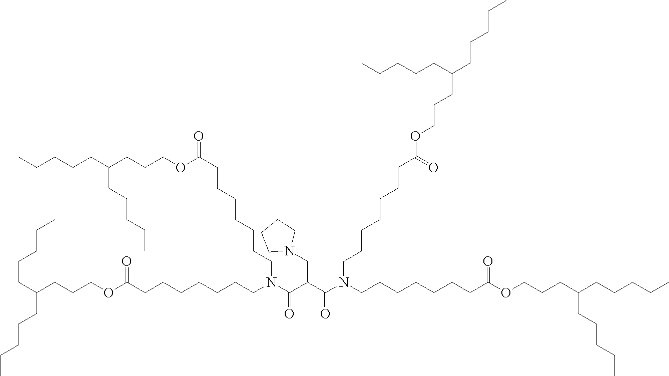 [SMILES: CCCCCC(CCCCC)CCCOC(=O)CCCCCCCN(CCCCCCCC(=O)OCCCC(CCCCC)CCCCC)C(=O)C(CN1CCCC1)C(=O)N(CCCCCCCC(=O)OCCCC(CCCCC)CCCCC)CCCCCCCC(=O)OCCCC(CCCCC)CCCCC] | tetrakis(4-pentylnonyl) 8,8′,8″,8″-((2- (pyrrolidin-1- ylmethyl)malonyl)bis(az anetriyl)) tetraoctanoate |
| | 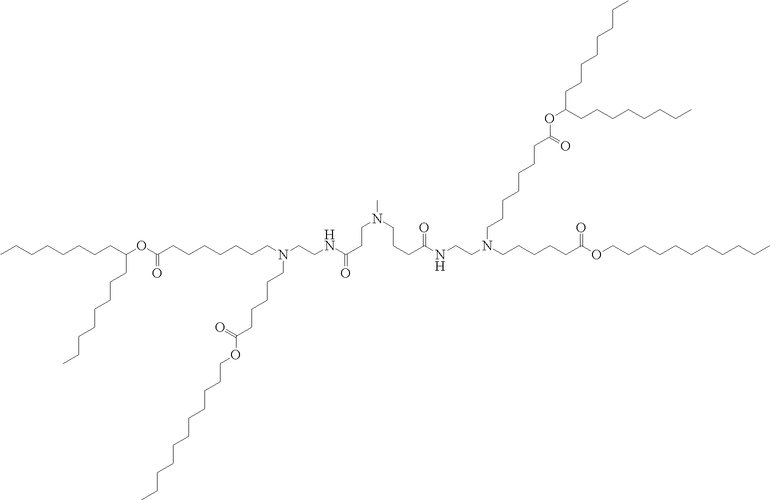 [SMILES: CCCCCCCCCCCOC(=O)CCCCCN(CCCCCCCC(=O)OC(CCCCCCCC)CCCCCCCC)CCNC(=O)CCCN(C)CCC(=O)NCCN(CCCCCCCC(=O)OC(CCCCCCCC)CCCCCCCC)CCCCCC(=O)OCCCCCCCCCCC] | di(heptadecan-9-yl) 16- methyl-13,20-dioxo- 9,24-bis(6-oxo-6- (undecyloxy)hexyl)- 9,12,16,21,24- pentaazadotriacontane dioate |
| | 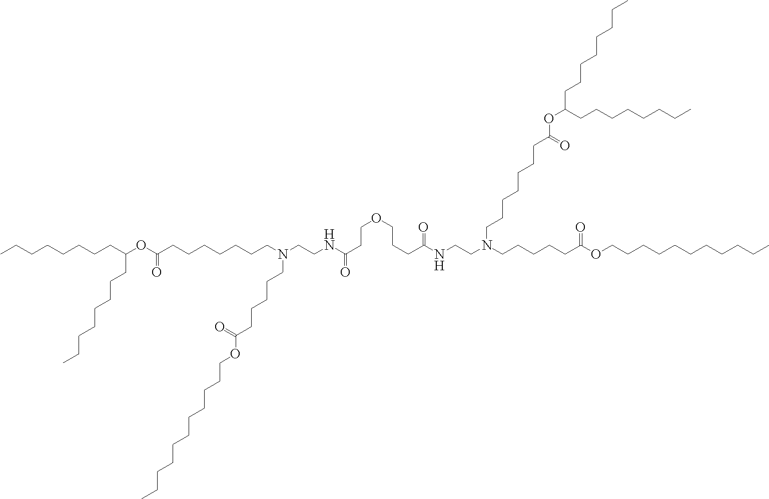 [SMILES: CCCCCCCCCCCOC(=O)CCCCCN(CCCCCCCC(=O)OC(CCCCCCCC)CCCCCCCC)CCNC(=O)CCCOCCC(=O)NCCN(CCCCCCCC(=O)OC(CCCCCCCC)CCCCCCCC)CCCCCC(=O)OCCCCCCCCCCC] | di(heptadecan-9-yl) 13,20-dioxo-9,24-bis(6- oxo-6- (undecyloxy)hexyl)-16- oxa-9,12,21,24- tetraazadotriacontanedioate |
| 2248 | 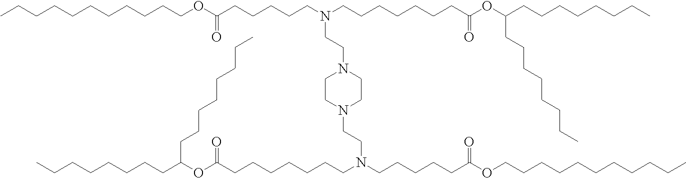 [SMILES: CCCCCCCCCCCOC(=O)CCCCCN(CCCCCCCC(=O)OC(CCCCCCCC)CCCCCCCC)CCN1CCN(CCN(CCCCCCCC(=O)OC(CCCCCCCC)CCCCCCCC)CCCCCC(=O)OCCCCCCCCCCC)CC1] | heptadecan-9-yl 8-({2- [4-(2-{[8-(heptadecan- 9-yloxy)-8-oxooctyl][6- oxo-6- (undecyloxy)hexyl] amino}ethyl)piperazin-1- ylethyl}[6-oxo-6- (undecyloxy)hexyl] amino)octanoate |
| 2190 | 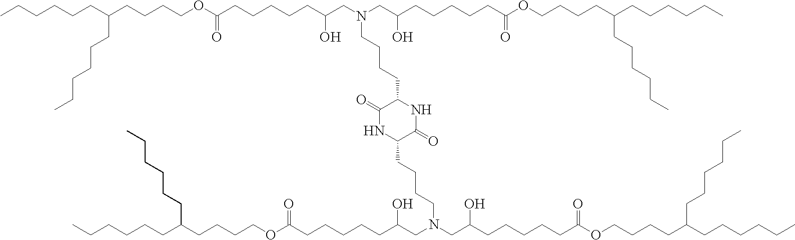 [SMILES: CCCCCCC(CCCCCC)CCCCOC(=O)CCCCCC(O)CN(CCCC[C@@H]1NC(=O)[C@H](CCCCN(CC(O)CCCCCC(=O)OCCCCC(CCCCCC)CCCCCC)CC(O)CCCCCC(=O)OCCCCC(CCCCCC)CCCCCC)NC1=O)CC(O)CCCCCC(=O)OCCCCC(CCCCCC)CCCCCC] | 5-hexylundecyl 8-({4- [(2S,5S)-5-{4-[bis({8- [(5-hexylundecyl)oxy]- 2-hydroxy-8- oxooctyl})amino]butyl}- 3,6-dioxopiperazin-2- yl]butyl}({8-[(5- hexylundecyl)oxy]-2- hydroxy-8- oxooctyl})amino)-7- hydroxyoctanoate |
| 2189 | 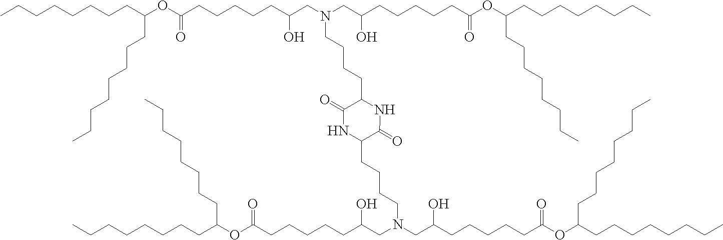 | heptadecan-9-yl 8-({4- [(2S,5S)-5-(4-{bis[8- (heptadecan-9-yloxy)-2- hydroxy-8- oxooctyl]amino}butyl)- 3,6-dioxopiperazin-2- yl]butyl}[8-(heptadecan- 9-yloxy)-2-hydroxy-8- oxooctyl]amino)-7- hydroxyoctanoate |
| 2287 | 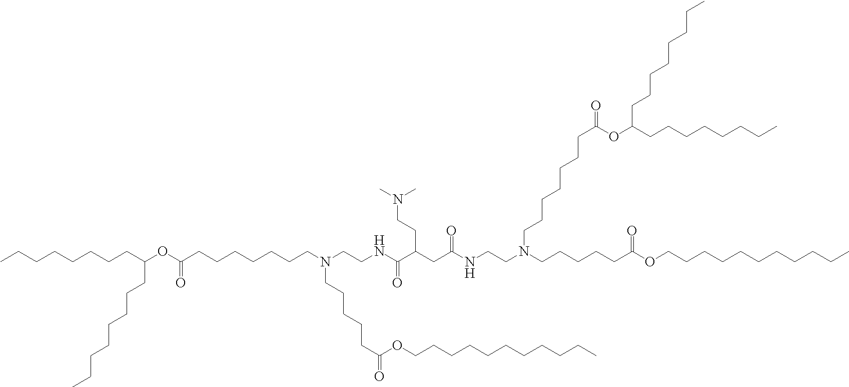 [SMILES: CCCCCCCCCCCOC(=O)CCCCCN(CCCCCCCC(=O)OC(CCCCCCCC)CCCCCCCC)CCNC(=O)CC(CCN(C)C)C(=O)NCCN(CCCCCCCC(=O)OC(CCCCCCCC)CCCCCCCC)CCCCCC(=O)OCCCCCCCCCCC] | di(heptadecan-9-yl) 14- (2- (dimethylamino)ethyl)- 13,16-dioxo-9,20-bis(6- oxo-6- (undecyloxy)hexyl)- 9,12,17,20- tetraazaoctacosanedioate |
| 2315 | 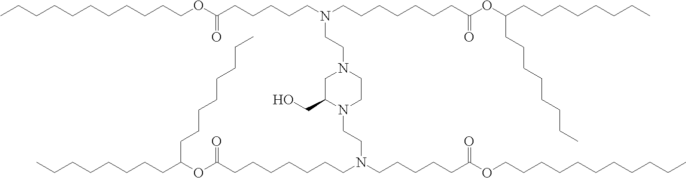 [SMILES: CCCCCCCCCCCOC(=O)CCCCCN(CCCCCCCC(=O)OC(CCCCCCCC)CCCCCCCC)CCN1CCN(CCN(CCCCCCCC(=O)OC(CCCCCCCC)CCCCCCCC)CCCCCC(=O)OCCCCCCCCCCC)[C@@H](CO)C1] | di(heptadecan-9-yl) 8,8′-(((2- (hydroxymethyl) piperazine- 1,4-diyl)bis(ethane- 2,1-diyl)) bis((6-oxo-6- (undecyloxy)hexyl) azanediyl)(R)-dioctanoate |
| 2316 | 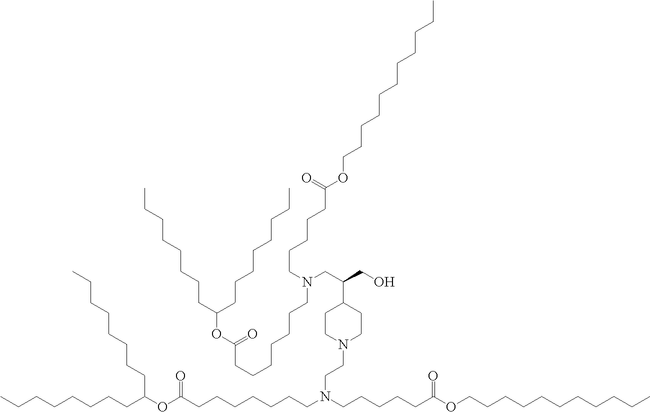 [SMILES: CCCCCCCCCCCOC(=O)CCCCCN(CCCCCCCC(=O)OC(CCCCCCCC)CCCCCCCC)CCN1CCC([C@H](CO)CN(CCCCCCCC(=O)OC(CCCCCCCC)CCCCCCCC)CCCCCC(=O)OCCCCCCCCCCC)CC1] | heptadecan-9-yl (S)-8- ((2-(4-(1-((8- (heptadecan-9-yloxy)-8- oxooctyl)(6-oxo-6- (undecyloxy)hexyl) amino)-3-hydroxypropan- 2-yl)piperazin-1- yl)ethyl)(6-oxo-6- (undecyloxy)hexyl) amino)octanoate |
| 2317 | 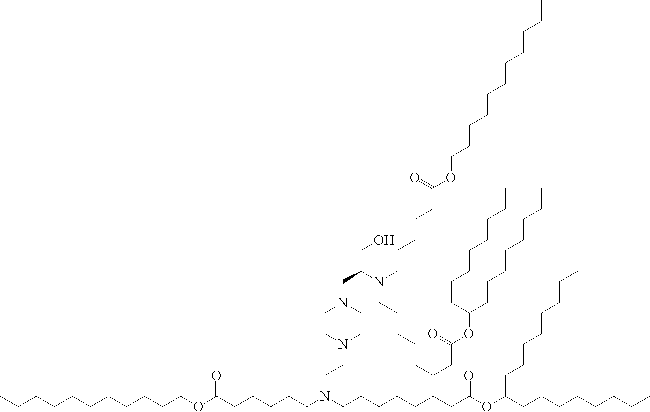 [SMILES: CCCCCCCCCCCOC(=O)CCCCCN(CCCCCCCC(=O)OC(CCCCCCCC)CCCCCCCC)CCN1CCN(C[C@@H](CO)N(CCCCCCCC(=O)OC(CCCCCCCC)CCCCCCCC)CCCCCC(=O)OCCCCCCCCCCC)CC1] | heptadecan-9-yl (S)-8- ((2-(4-(2-((8- (heptadecan-9-yloxy)-8- oxooctyl)(6-oxo-6- (undecyloxy)hexyl) amino)-3- hydroxypropyl)piperazin- 1-yl)ethyl)(6-oxo-6- (undecyloxy)hexyl) amino)octanoate |
| 2318 | 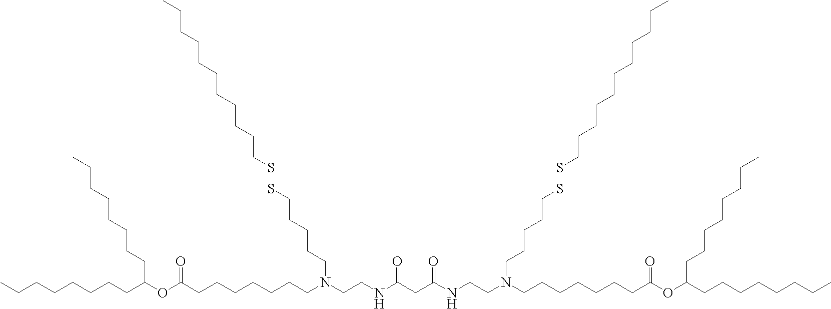 | di(heptadecan-9-yl) 13,15-dioxo-9,19-bis(5- (undecyldisulfaneyl) pentyl)-9,12,16,19- tetraazaheptacosanedioate |
| 2439 | 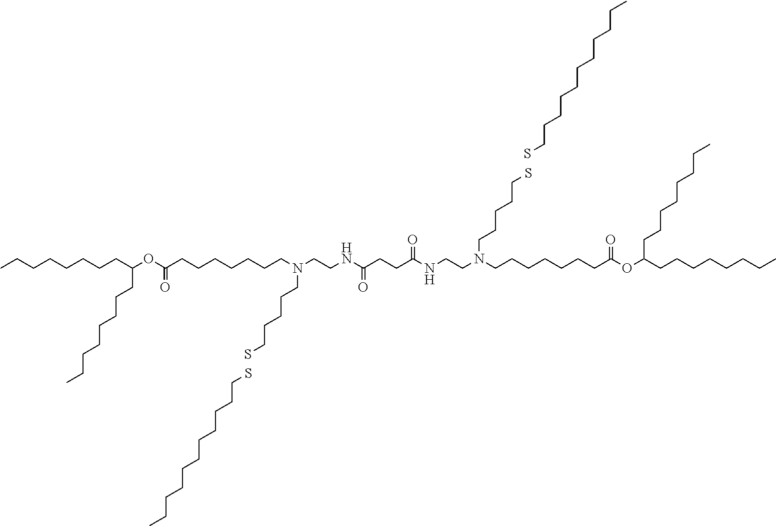 | di(heptadecan-9-yl) 13,16-dioxo-9,20-bis(5- (undecyldisulfaneyl) pentyl)-9,12,17,20- tetraazaoctacosanedioate |
| 2319 | 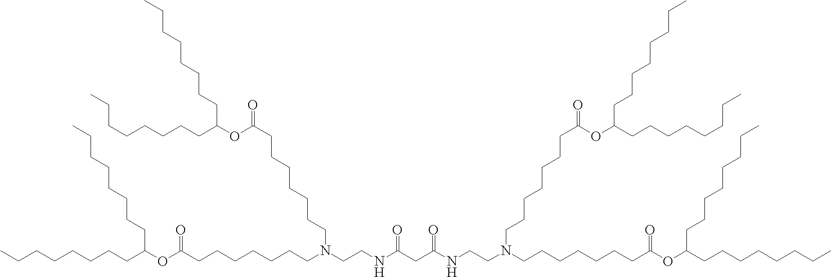 [SMILES: CCCCCCCCC(CCCCCCCC)OC(=O)CCCCCCCN(CCCCCCCC(=O)OC(CCCCCCCC)CCCCCCCC)CCNC(=O)CC(=O)NCCN(CCCCCCCC(=O)OC(CCCCCCCC)CCCCCCCC)CCCCCCCC(=O)OC(CCCCCCCC)CCCCCCCC] | di(heptadecan-9-yl) 9,19-bis(8- (heptadecan-9-yloxy)-8- oxooctyl)-13, 15-dioxo- 9,12,16,19- tetraazaheptacosanedioate |
| 2320 | 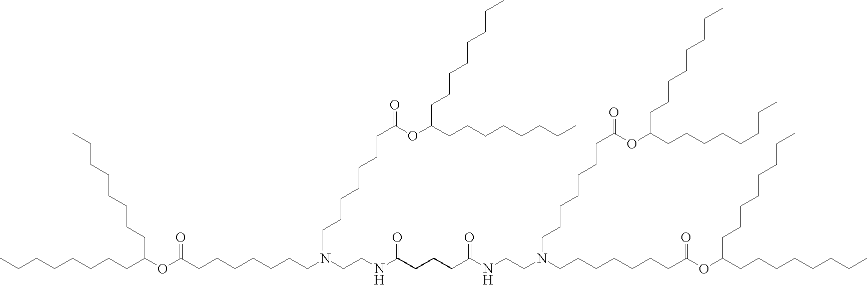 | di(heptadecan-9-yl) 9,21-bis(8- (heptadecan-9-yloxy)-8- oxooctyl)-13,17-dioxo- 9,12,18,21- tetraazanonacosanedioate |
| 2321 | 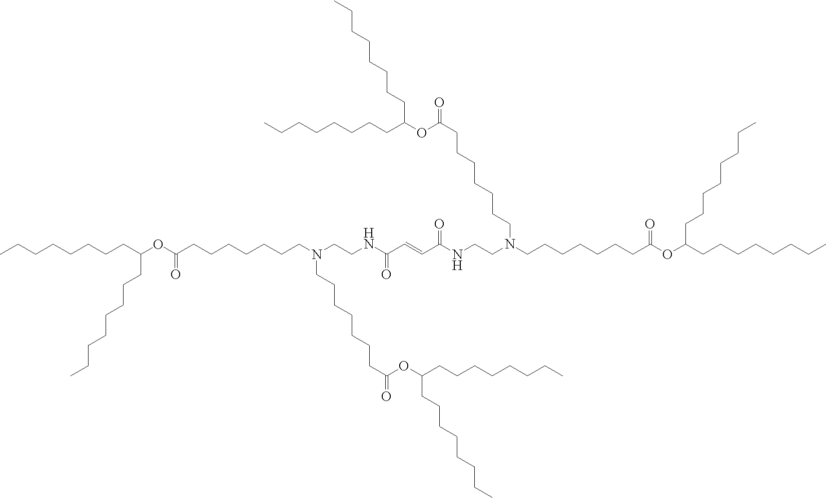 [SMILES: CCCCCCCCC(CCCCCCCC)OC(=O)CCCCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCCCCC(=O)OC(CCCCCCCC)CCCCCCCC] | di(heptadecan-9-yl) (E)- 9,20-bis(8- (heptadecan-9-yloxy)-8- oxooctyl)-13, 16-dioxo- 9,12,17,20- tetraazaoctacos-14- enedioate |
| 2322 | 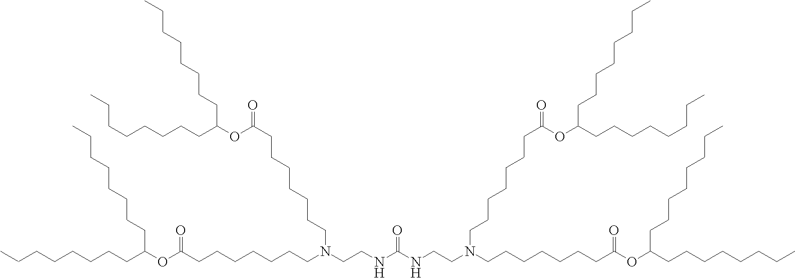 [SMILES: CCCCCCCCC(CCCCCCCC)OC(=O)CCCCCCCN(CCCCCCCC(=O)OC(CCCCCCCC)CCCCCCCC)CCNC(=O)NCCN(CCCCCCCC(=O)OC(CCCCCCCC)CCCCCCCC)CCCCCCCC(=O)OC(CCCCCCCC)CCCCCCCC] | di(heptadecan-9-yl) 9,17-bis(8- (heptadecan-9-yloxy)-8- oxooctyl)-13-oxo- 9,12,14,17- tetraazapentacosanedioate |
| 2323 | 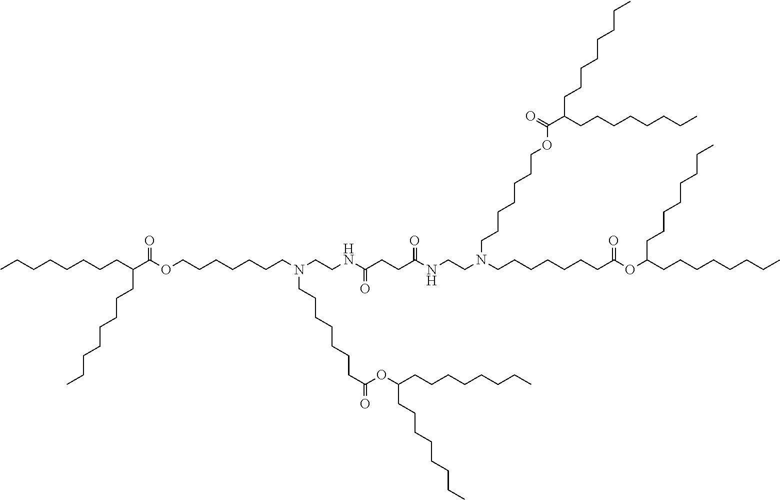 [SMILES: CCCCCCCCC(CCCCCCCC)OC(=O)CCCCCCCN(CCCCCCCOC(=O)C(CCCCCCCC)CCCCCCCC)CCNC(=O)CCC(=O)NCCN(CCCCCCCOC(=O)C(CCCCCCCC)CCCCCCCC)CCCCCCCC(=O)OC(CCCCCCCC)CCCCCCCC] | di(heptadecan-9-yl) 9,20-bis(7-((2- octyldecanoyl)oxy) heptyl)-13,16-dioxo- 9,12,17,20- tetraazaoctacosanedioate |
| 2324 | 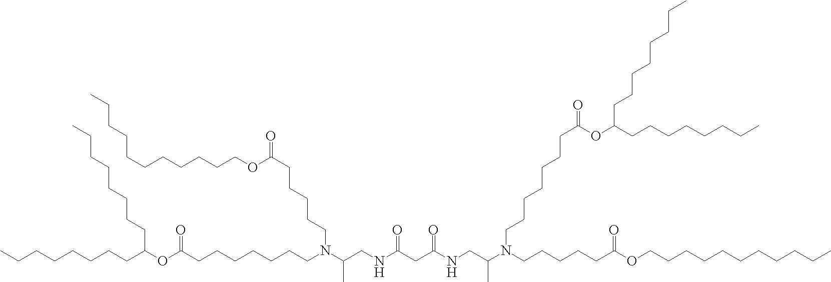 [SMILES: CCCCCCCCCCCOC(=O)CCCCCN(CCCCCCCC(=O)OC(CCCCCCCC)CCCCCCCC)C(C)CNC(=O)CC(=O)NCC(C)N(CCCCCCCC(=O)OC(CCCCCCCC)CCCCCCCC)CCCCCC(=O)OCCCCCCCCCCC] | di(heptadecan-9-yl) 10,18-dimethyl-13,15- dioxo-9,19-bis(6-oxo-6- (undecyloxy)hexyl)- 9,12,16,19- tetraazaheptacosanedioate |
| 2325 | 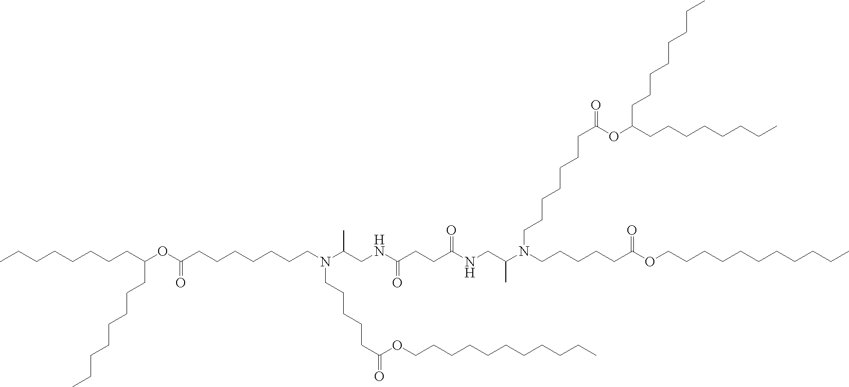 [SMILES: CCCCCCCCCCCOC(=O)CCCCCN(CCCCCCCC(=O)OC(CCCCCCCC)CCCCCCCC)C(C)CNC(=O)CCC(=O)NCC(C)N(CCCCCCCC(=O)OC(CCCCCCCC)CCCCCCCC)CCCCCC(=O)OCCCCCCCCCCC] | di(heptadecan-9-yl) 10,19-dimethyl-13,16- dioxo-9,20-bis(6-oxo-6- (undecyloxy)hexyl)- 9,12,17,20- tetraazaoctacosanedioate |
| 2326 | 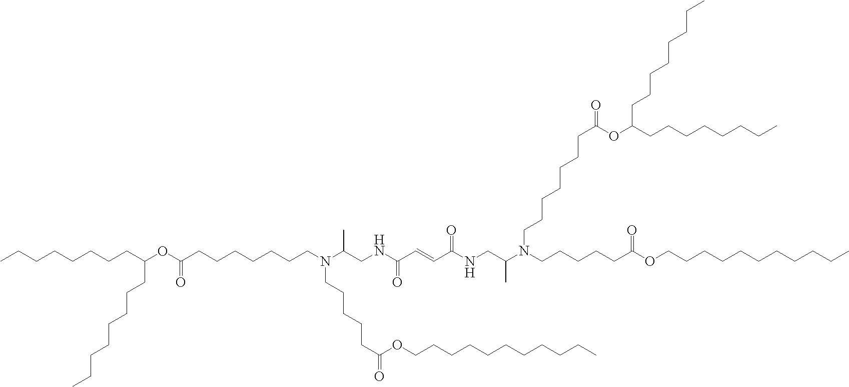 [SMILES: CCCCCCCCCCCOC(=O)CCCCCN(CCCCCCCC(=O)OC(CCCCCCCC)CCCCCCCC)C(C)CNC(=O)/C=C/C(=O)NCC(C)N(CCCCCCCC(=O)OC(CCCCCCCC)CCCCCCCC)CCCCCC(=O)OCCCCCCCCCCC] | di(heptadecan-9-yl) (E)- 10,19-dimethyl-13,16- dioxo-9,20-bis(6-oxo-6- (undecyloxy)hexyl)- 9,12,17,20- tetraazaoctacos-14- enedioate |
| 2327 | 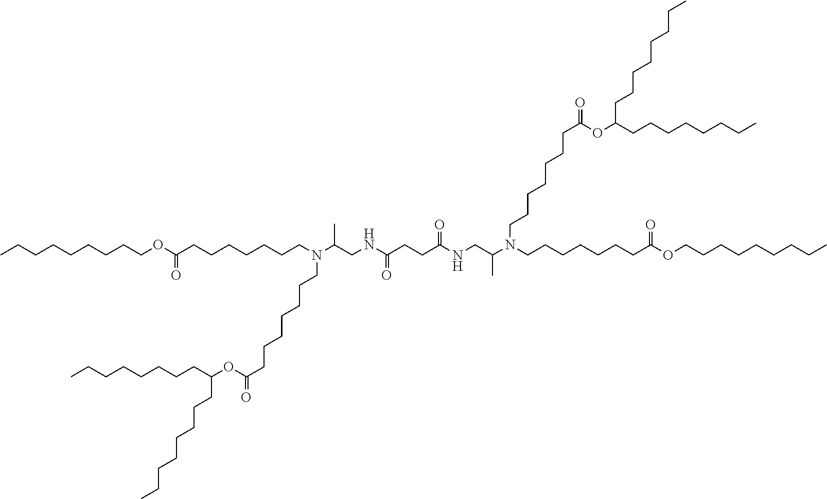 [SMILES: CCCCCCCCCOC(=O)CCCCCCCN(CCCCCCCC(=O)OC(CCCCCCCC)CCCCCCCC)C(C)CNC(=O)CCC(=O)NCC(C)N(CCCCCCCC(=O)OCCCCCCCCC)CCCCCCCC(=O)OC(CCCCCCCC)CCCCCCCC] | di(heptadecan-9-yl) 10,19-dimethyl-9,20- bis(8-(nonyloxy)-8- oxooctyl)-13,16-dioxo- 9,12,17,20- tetraazaoctacosanedioa te |
| 2328 | 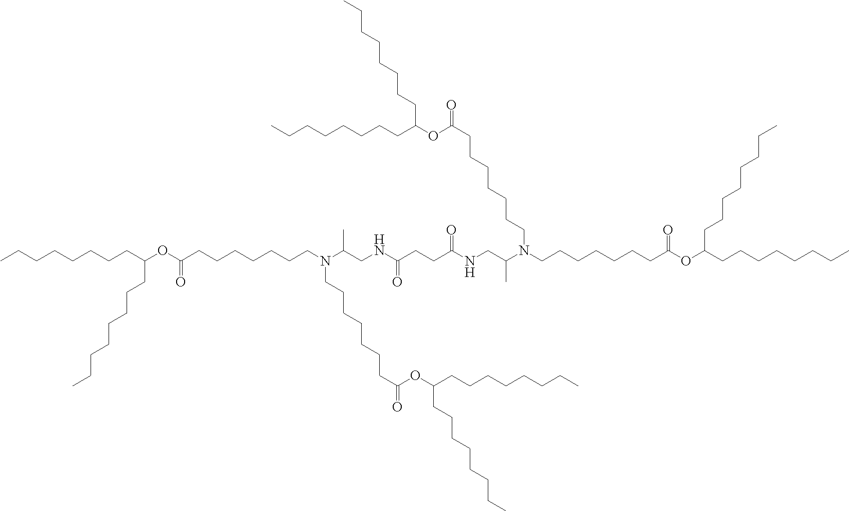 [SMILES: CCCCCCCCC(CCCCCCCC)OC(=O)CCCCCCCN(CCCCCCCC(=O)OC(CCCCCCCC)CCCCCCCC)C(C)CNC(=O)CCC(=O)NCC(C)N(CCCCCCCC(=O)OC(CCCCCCCC)CCCCCCCC)CCCCCCCC(=O)OC(CCCCCCCC)CCCCCCCC] | di(heptadecan-9-yl) 9,20-bis(8- (heptadecan-9-yloxy)-8- oxooctyl)-10,19- dimethyl-13,16-dioxo- 9,12,17,20- tetraazaoctacosanedioa te |
| 2332 | 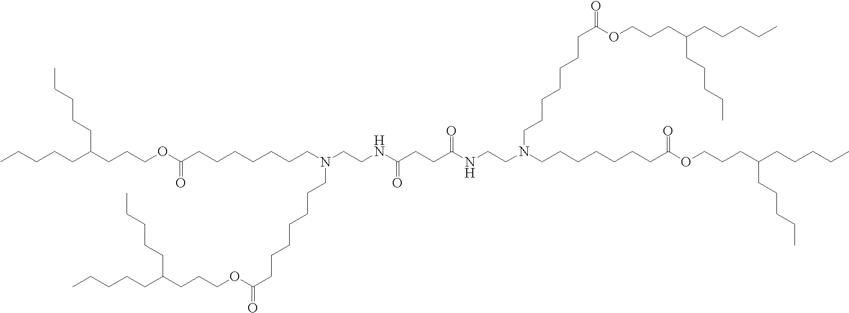 [SMILES: CCCCCC(CCCCC)CCCOC(=O)CCCCCCCN(CCCCCCCC(=O)OCCCC(CCCCC)CCCCC)CCNC(=O)CCC(=O)NCCN(CCCCCCCC(=O)OCCCC(CCCCC)CCCCC)CCCCCCCC(=O)OCCCC(CCCCC)CCCCC] | bis(4-pentylnonyl) 13,16-dioxo-9,20-bis(8- oxo-8-((4- pentylnonyl)oxy)octyl)- 9,12,17,20- tetraazaoctacosanedioate |
| 2334 | 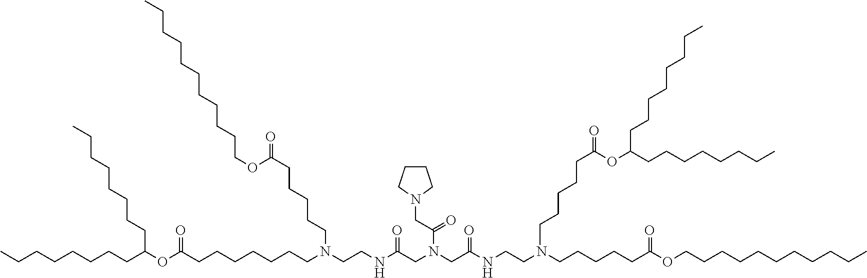 | di(heptadecan-9-yl) 13,17-dioxo-9,21-bis(6- oxo-6- (undecyloxy)hexyl)-15- (2-(pyrrolidin-1- yl)acetyl)- 9,12,15,18,21- pentaazanonacosanedioate |
| 2353 | 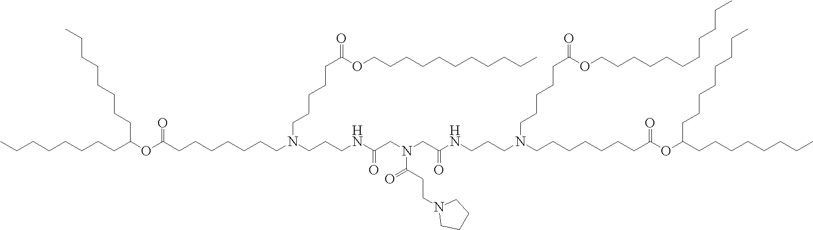 [SMILES: CCCCCCCCCCCOC(=O)CCCCCN(CCCCCCCC(=O)OC(CCCCCCCC)CCCCCCCC)CCCNC(=O)CN(CC(=O)NCCCN(CCCCCCCC(=O)OC(CCCCCCCC)CCCCCCCC)CCCCCC(=O)OCCCCCCCCCCC)C(=O)CCN1CCCC1] | di(heptadecan-9-yl) 14,18-dioxo-9,23-bis(6- oxo-6- (undecyloxy)hexyl)-16- (3-(pyrrolidin-1- yl)propanoyl)- 9,13,16,19,23- pentaazahentria- contanedioate |
| 2354 | 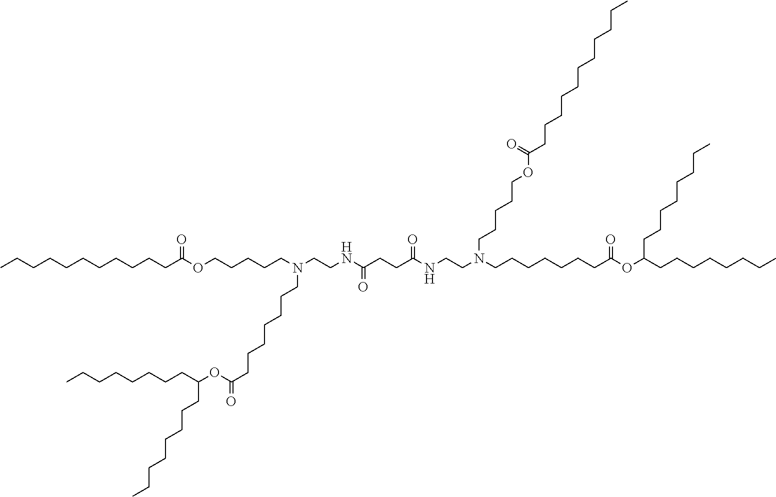 [SMILES: CCCCCCCCCCCC(=O)OCCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCOC(=O)CCCCCCCCCCC] | di(heptadecan-9-yl) 9,20-bis(5- (dodecanoyloxy)pentyl)- 13,16-dioxo- 9,12,17,20- tetraazaoctacosanedioate |
| 2355 | 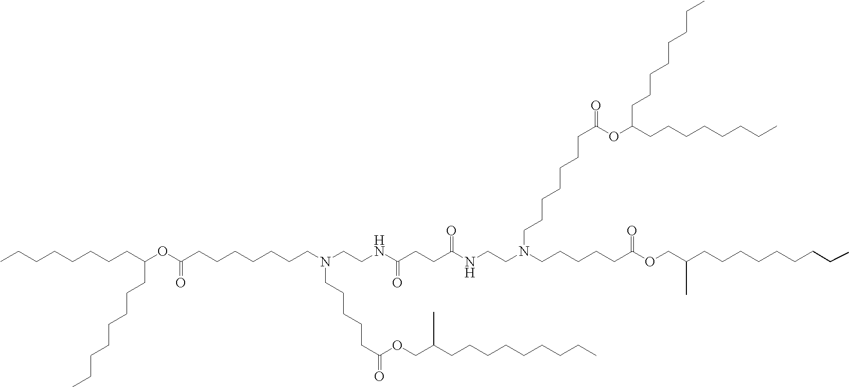 [SMILES: CCCCCCCCCC(C)COC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCC(=O)OCC(C)CCCCCCCCC] | di(heptadecan-9-yl) 9,20-bis(6-((2- methylundecyl)oxy)-6- oxohexyl)-13,16-dioxo- 9,12,17,20- tetraazaoctacosanedioate |
| 2356 | 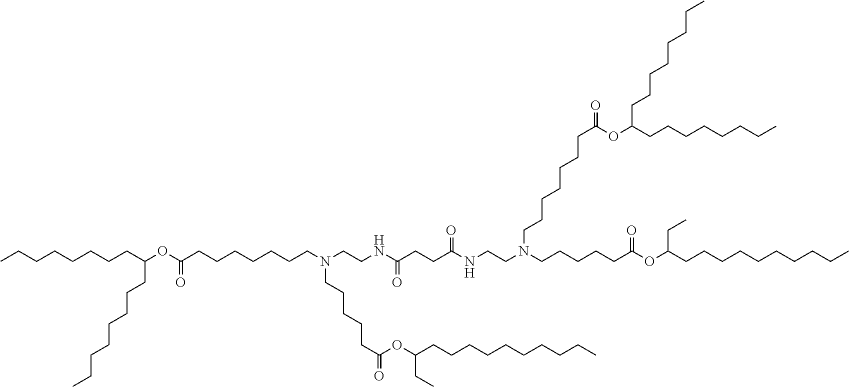 [SMILES: CCCCCCCCCCC(CC)OC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCC(=O)OC(CC)CCCCCCCCCC] | di(heptadecan-9-yl) 13,16-dioxo-9,20-bis(6- oxo-6-(tridecan-3- yloxy)hexyl)- 9,12,17,20- tetraazaoctacosanedioate |
| 2357 | 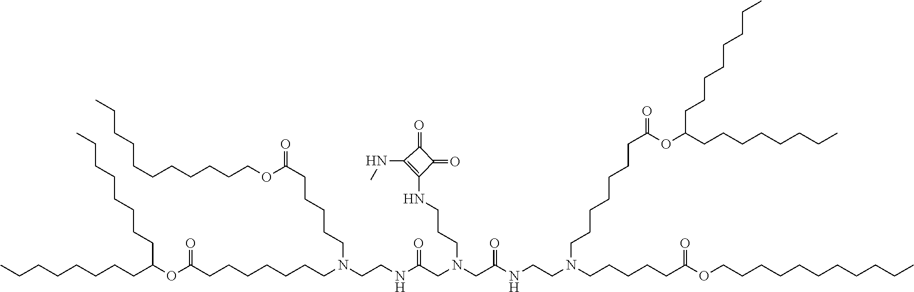 [SMILES: CCCCCCCCCCCOC(=O)CCCCCN(CCCCCCCC(=O)OC(CCCCCCCC)CCCCCCCC)CCNC(=O)CN(CCCNc1c(NC)c(=O)c1=O)CC(=O)NCCN(CCCCCCCC(=O)OC(CCCCCCCC)CCCCCCCC)CCCCCC(=O)OCCCCCCCCCCC] | di(heptadecan-9-yl) 15- (3-((2-(methylamino)- 3,4-dioxocyclobut-1-en- 1-yl)amino)propyl)- 13,17-dioxo-9,21-bis(6- oxo-6- (undecyloxy)hexyl)- 9,12,15,18,21- pentaazanonacosanedioate |
| 2358 | 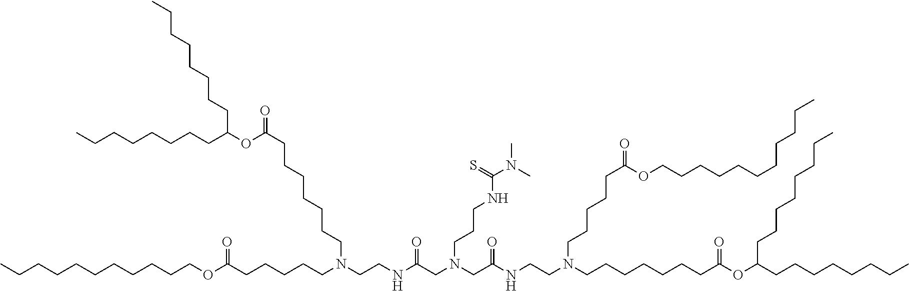 [SMILES: CCCCCCCCCCCOC(=O)CCCCCN(CCCCCCCC(=O)OC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCCC(=O)OCCCCCCCCCCC] | di(heptadecan-9-yl) 15- (3-(3,3- dimethylthioureido) propyl)-13,17-dioxo- 9,21-bis(6-oxo-6- (undecyloxy)hexyl)- 9,12,15,18,21- pentaazanonacosanedioate |
| 2361 | 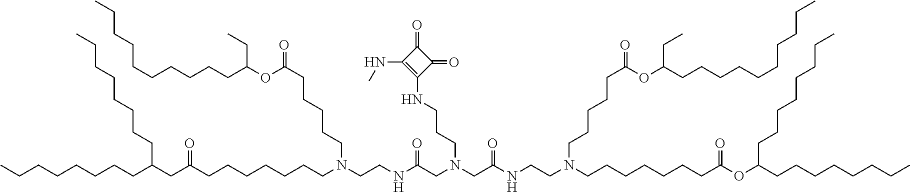 [SMILES: CCCCCCCCCCC(CC)OC(=O)CCCCCN(CCCCCCCC(=O)CC(CCCCCCCC)CCCCCCCC)CCNC(=O)CN(CCCNc1c(NC)c(=O)c1=O)CC(=O)NCCN(CCCCCCCC(=O)OC(CCCCCCCC)CCCCCCCC)CCCCCC(=O)OC(CC)CCCCCCCCCC] | di(heptadecan-9-yl) 15- (3-((2-(methylamino)- 3,4-dioxocyclobut-1-en- 1-yl)amino)propyl)- 13,17-dioxo-9,21-bis(6- oxo-6-(tridecan-3- yloxy)hexyl)- 9,12,15,18,21- pentaazanonacosanedioate |
| 2362 | 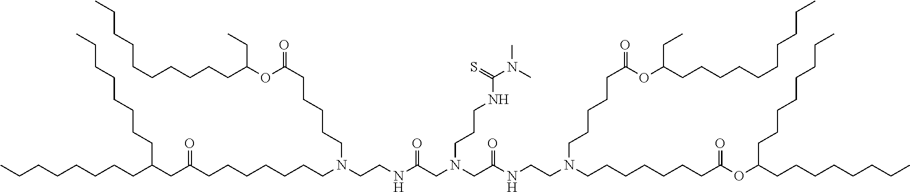 [SMILES: CCCCCCCCCCC(CC)OC(=O)CCCCCN(CCCCCCCC(=O)CC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCCC(=O)OC(CC)CCCCCCCCCC] | di(heptadecan-9-yl) 15- (3-(3,3- dimethylthioureido) propyl)-13,17-dioxo-9,21- bis(6-oxo-6-(tridecan-3- yloxy)hexyl)- 9,12,15,18,21- pentaazanonacosanedioate |
| 2378 | 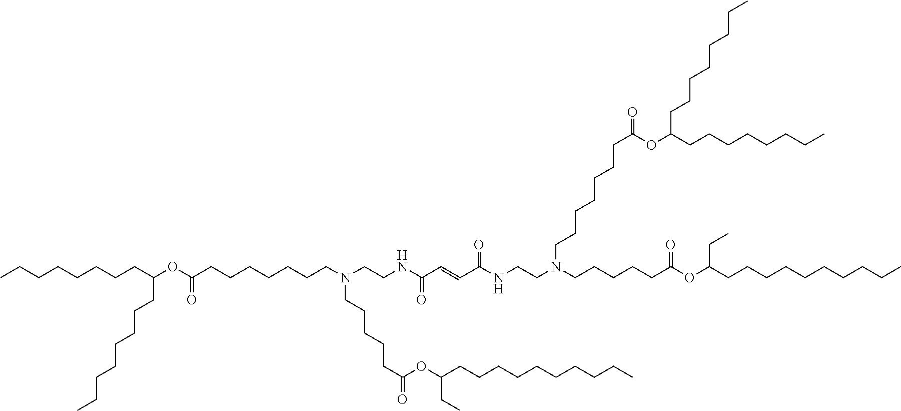 [SMILES: CCCCCCCCCCC(CC)OC(=O)CCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCCC(=O)OC(CC)CCCCCCCCCC] | di(heptadecan-9-yl) (E)- 13,16-dioxo-9,20-bis(6- oxo-6-(tridecan-3- yloxy)hexyl)- 9,12,17,20- tetraazaoctacos-14- enedioate |
| 2379 | 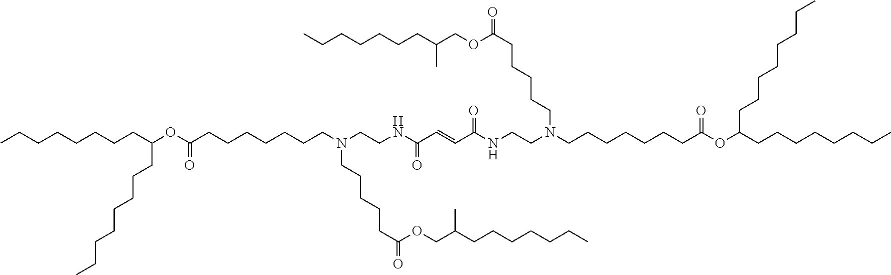 [SMILES: CCCCCCCCC(CCCCCCCC)OC(=O)CCCCCCCN(CCCCCC(=O)OCC(C)CCCCCCC)CCNC(=O)/C=C/C(=O)NCCN(CCCCCCCC(=O)OC(CCCCCCCC)CCCCCCCC)CCCCCC(=O)OCC(C)CCCCCCC] | di(heptadecan-9-yl) (E)- 9,20-bis(6-((2- methylnonyl)oxy)-6- oxohexyl)-13, 16-dioxo- 9,12,17,20- tetraazaoctacos-14- enedioate |
| 2380 | 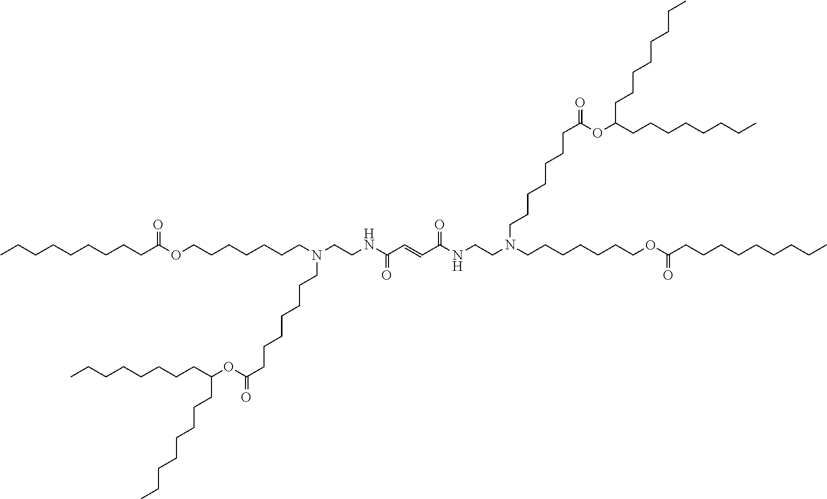 [SMILES: CCCCCCCCCC(=O)OCCCCCCCN(CCCCCCCC(=O)OC(CCCCCCCC)CCCCCCCC)CCNC(=O)/C=C/C(=O)NCCN(CCCCCCCOC(=O)CCCCCCCCC)CCCCCCCC(=O)OC(CCCCCCCC)CCCCCCCC] | di(heptadecan-9-yl) (E)- 9,20-bis(7- (decanoyloxy)heptyl)- 13,16-dioxo-9,12,17,20- tetraazaoctacos-14- enedioate |
| 2381 | 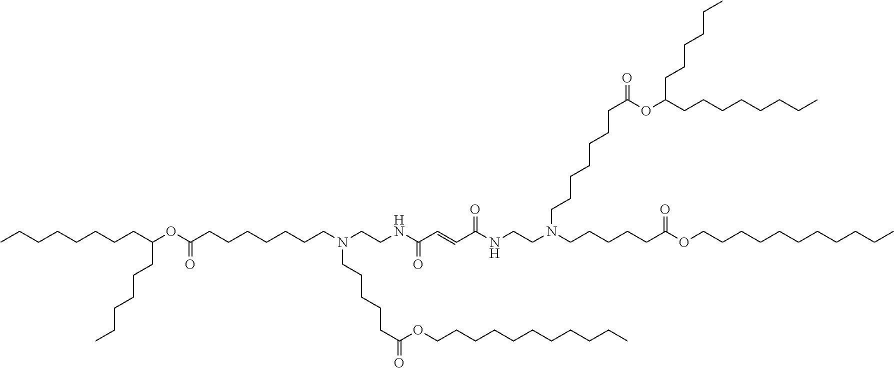 [SMILES: CCCCCCCCCCCOC(=O)CCCCCN(CCCCCCCC(=O)OC(CCCCCC)CCCCCCCC)CCNC(=O)/C=C/C(=O)NCCN(CCCCCCCC(=O)OC(CCCCCC)CCCCCCCC)CCCCCC(=O)OCCCCCCCCCCC] | di(pentadecan-7-yl) (E)- 13,16-dioxo-9,20-bis(6- oxo-6- (undecyloxy)hexyl)- 9,12,17,20- tetraazaoctacos-14- enedioate |
| 2382 | 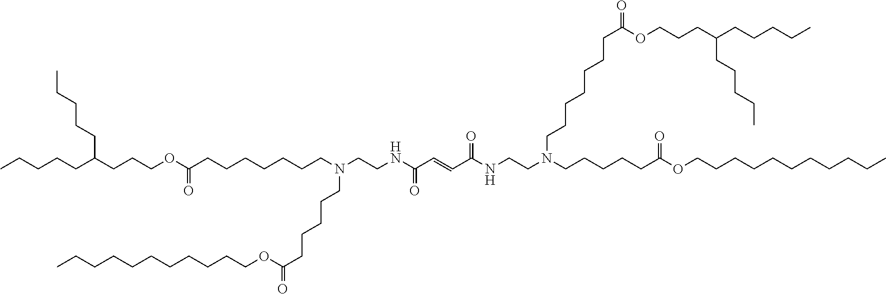 [SMILES: CCCCCCCCCCCOC(=O)CCCCCN(CCCCCCCC(=O)OCCCC(CCCCC)CCCCC)CCNC(=O)/C=C/C(=O)NCCN(CCCCCCCC(=O)OCCCC(CCCCC)CCCCC)CCCCCC(=O)OCCCCCCCCCCC] | bis(4-pentylnonyl) (E)- 13,16-dioxo-9,20-bis(6- oxo-6- (undecyloxy)hexyl)- 9,12,17,20- tetraazaoctacos-14- enedioate |
| 2384 | 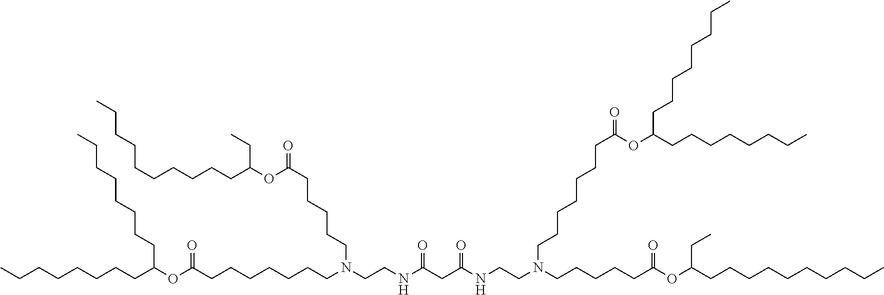 [SMILES: CCCCCCCCCCC(CC)OC(=O)CCCCCN(CCCCCCCC(=O)OC(CCCCCCCC)CCCCCCCC)CCNC(=O)CC(=O)NCCN(CCCCCCCC(=O)OC(CCCCCCCC)CCCCCCCC)CCCCCC(=O)OC(CC)CCCCCCCCCC] | di(heptadecan-9-yl) 13,15-dioxo-9,19-bis(6- oxo-6-(tridecan-3- yloxy)hexyl)- 9,12,16,19- tetraazaheptacosanedioate |
| 2385 | 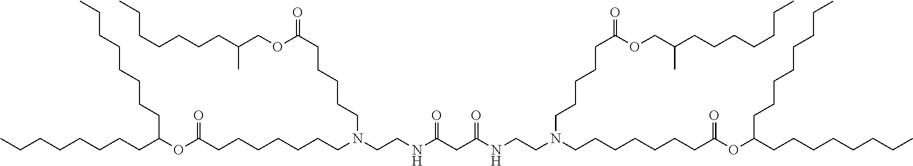 [SMILES: CCCCCCCCC(CCCCCCCC)OC(=O)CCCCCCCN(CCCCCC(=O)OCC(C)CCCCCCC)CCNC(=O)CC(=O)NCCN(CCCCCCCC(=O)OC(CCCCCCCC)CCCCCCCC)CCCCCC(=O)OCC(C)CCCCCCC] | di(heptadecan-9-yl) 9,19-bis(6-((2- methylnonyl)oxy)-6- oxohexyl)-13, 15-dioxo- 9,12,16,19- tetraazaheptacosanedi oate |
| 2386 | 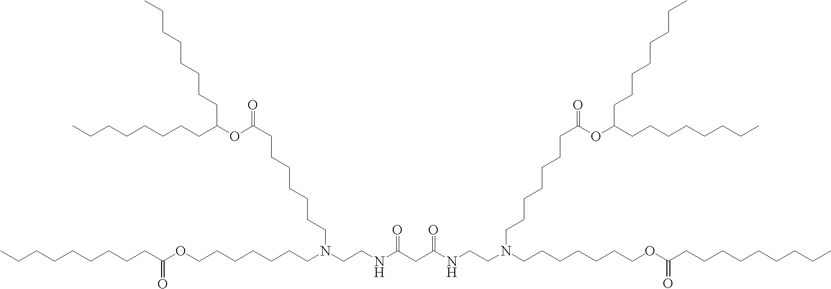 [SMILES: CCCCCCCCCC(=O)OCCCCCCCN(CCCCCCCC(=O)OC(CCCCCCCC)CCCCCCCC)CCNC(=O)CC(=O)NCCN(CCCCCCCOC(=O)CCCCCCCCC)CCCCCCCC(=O)OC(CCCCCCCC)CCCCCCCC] | di(heptadecan-9-yl) 9,19-bis(7- (decanoyloxy)heptyl)- 13,15-dioxo-9,12,16,19- tetraazaheptacosanedi oate |
| 2387 | 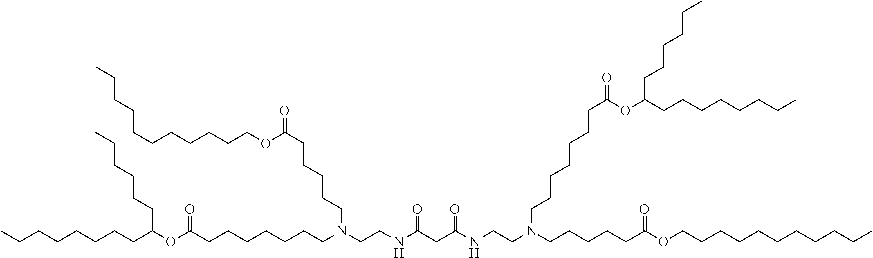 [SMILES: CCCCCCCCCCCOC(=O)CCCCCN(CCCCCCCC(=O)OC(CCCCCC)CCCCCCCC)CCNC(=O)CC(=O)NCCN(CCCCCCCC(=O)OC(CCCCCC)CCCCCCCC)CCCCCC(=O)OCCCCCCCCCCC] | di(pentadecan-7-yl) 13,15-dioxo-9,19-bis(6- oxo-6- (undecyloxy)hexyl)- 9,12,16,19- tetraazaheptacosanedi oate |
| 2388 | 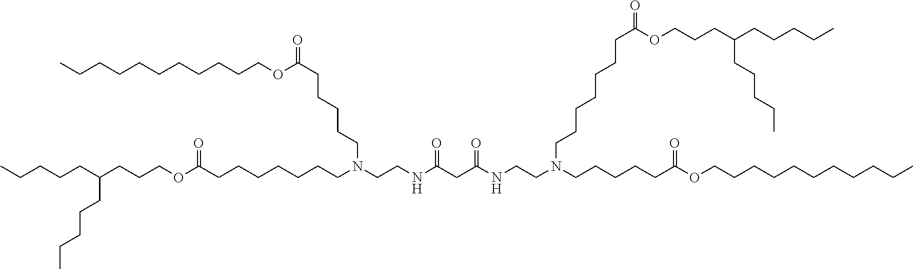 [SMILES: CCCCCCCCCCCOC(=O)CCCCCN(CCCCCCCC(=O)OCCCC(CCCCC)CCCCC)CCNC(=O)CC(=O)NCCN(CCCCCCCC(=O)OCCCC(CCCCC)CCCCC)CCCCCC(=O)OCCCCCCCCCCC] | bis(4-pentylnonyl) 13,15-dioxo-9,19-bis(6- oxo-6- (undecyloxy)hexyl)- 9,12,16,19- tetraazaheptacosanedi oate |
| 2389 | 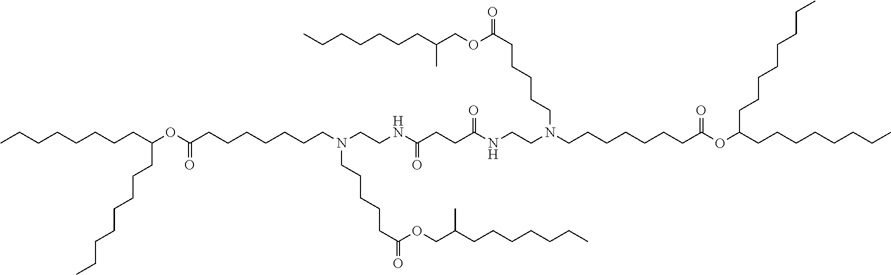 [SMILES: CCCCCCCCC(CCCCCCCC)OC(=O)CCCCCCCN(CCCCCC(=O)OCC(C)CCCCCCC)CCNC(=O)CCC(=O)NCCN(CCCCCCCC(=O)OC(CCCCCCCC)CCCCCCCC)CCCCCC(=O)OCC(C)CCCCCCC] | di(heptadecan-9-yl) 9,20-bis(6-((2- methylnonyl)oxy)-6- oxohexyl)-13,16-dioxo- 9,12,17,20- tetraazaoctacosanedioate |
| 2390 | 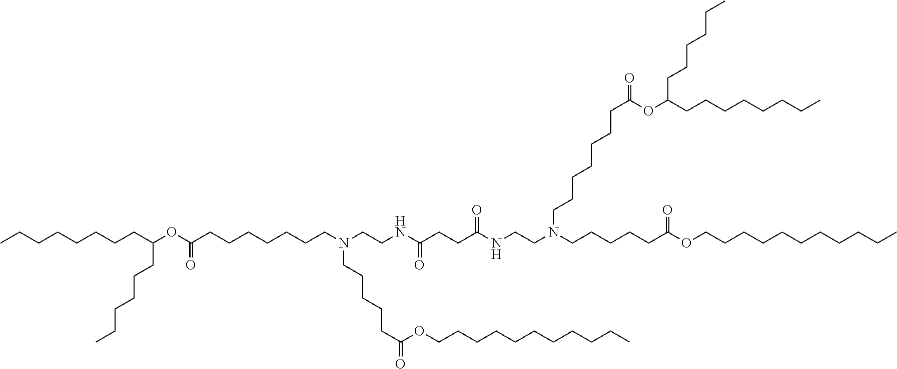 [SMILES: CCCCCCCCCCCOC(=O)CCCCCN(CCCCCCCC(=O)OC(CCCCCC)CCCCCCCC)CCNC(=O)CCC(=O)NCCN(CCCCCCCC(=O)OC(CCCCCC)CCCCCCCC)CCCCCC(=O)OCCCCCCCCCCC] | di(pentadecan-7-yl) 13,16-dioxo-9,20-bis(6- oxo-6- (undecyloxy)hexyl)- 9,12,17,20- tetraazaoctacosanedioate |
| 2391 | 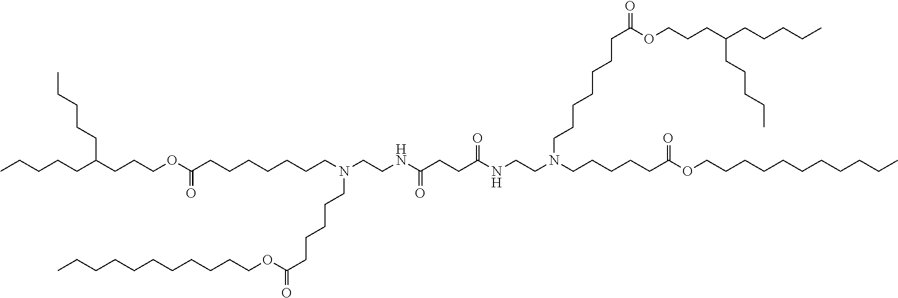 [SMILES: CCCCCCCCCCCOC(=O)CCCCCN(CCCCCCCC(=O)OCCCC(CCCCC)CCCCC)CCNC(=O)CCC(=O)NCCN(CCCCCCCC(=O)OCCCC(CCCCC)CCCCC)CCCCCC(=O)OCCCCCCCCCCC] | bis(4-pentylnonyl) 13,16-dioxo-9,20-bis(6- oxo-6- (undecyloxy)hexyl)- 9,12,17,20- tetraazaoctacosanedioate |
| 2393 | 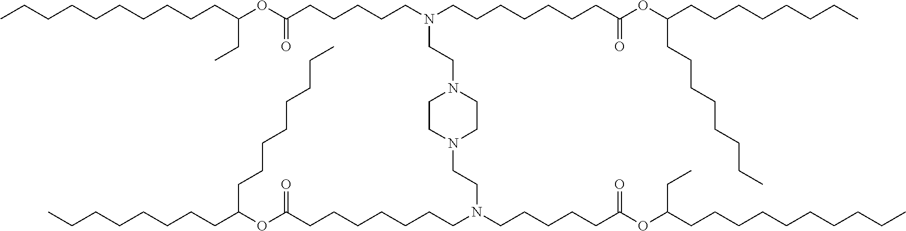 | di(heptadecan-9-yl) 8,8′-((piperazine-1,4- diylbis(ethane-2,1- diyl))bis((6-oxo-6- (tridecan-3- yloxy)hexyl)azanediyl)) dioctanoate |
| 2394 | 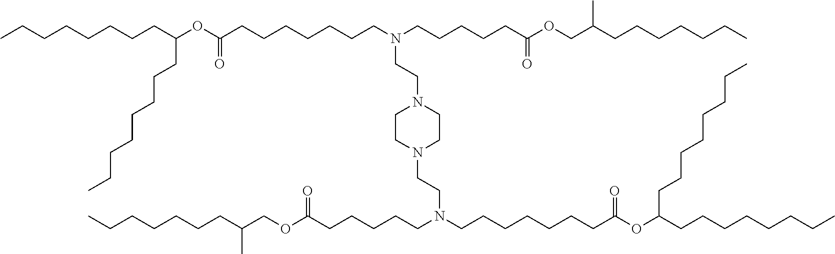 | di(heptadecan-9-yl) 8,8′-((piperazine-1,4- diylbis(ethane-2,1- diyl)) bis ((6-((2- methylnonyl)oxy)-6- oxohexyl)azanediyl)) dioctanoate |
| 2395 | 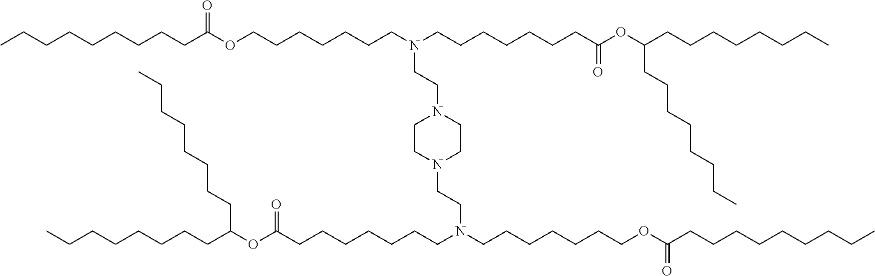 | ((piperazine-1,4- diylbis(ethane-2,1- diyl)) bis((8- (heptadecan-9-yloxy)-8- oxooctyl)azanediyl)) bis(heptane-7,1-diyl) bis(decanoate) |
| 2396 | 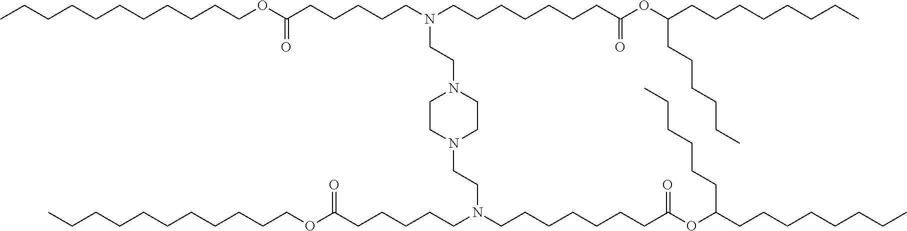 | di(pentadecan-7-yl) 8,8′-((piperazine-1,4- diylbis(ethane-2,1- diyl))bis((6-oxo-6- (undecyloxy)hexyl) azanediyl))dioctanoate |
| 2397 | 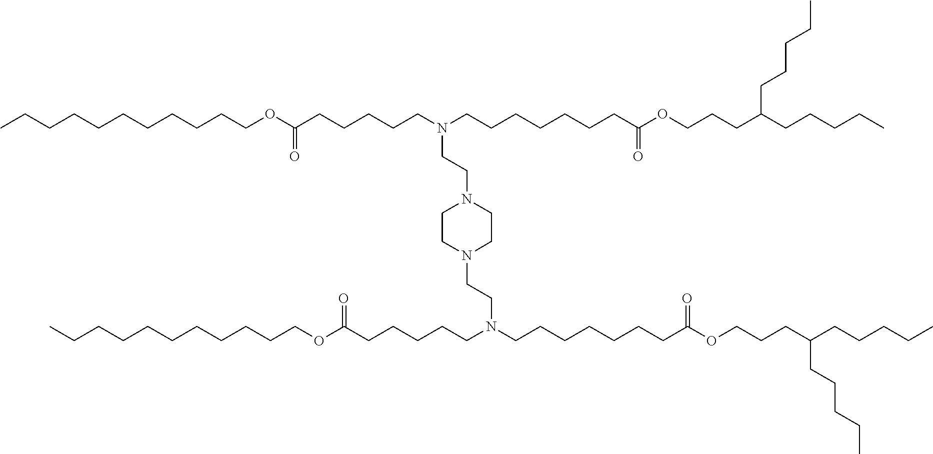 | bis(4-pentylnonyl) 8,8′- ((piperazine-1,4- diylbis(ethane-2,1- diyl))bis((6-oxo-6- (undecyloxy)hexyl) azanediyl))dioctanoate |
| 2399 | 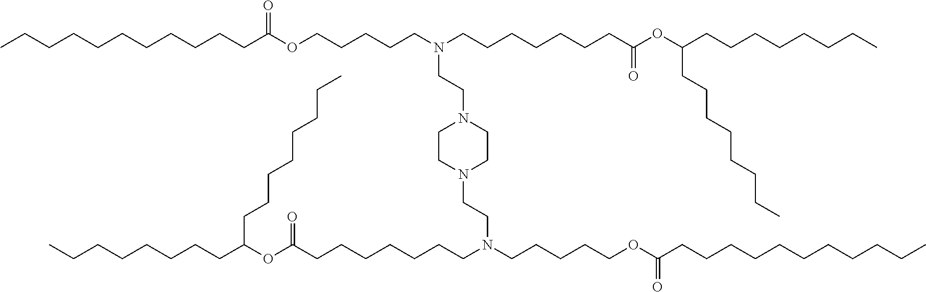 | ((piperazine-1,4- diylbis(ethane-2,1- diyl)) bis((8- (heptadecan-9-yloxy)-8- oxooctyl)azanediyl)) bis(pentane-5,1-diyl) didodecanoate |
| 2400 | 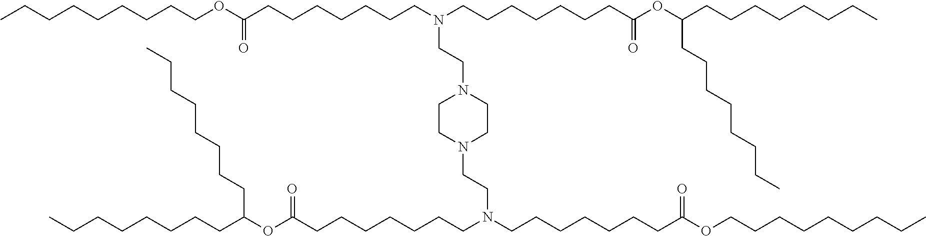 | di(heptadecan-9-yl) 8,8′-((piperazine-1,4- diylbis(ethane-2,1- diyl)) bis((8-(nonyloxy)- 8-oxooctyl)azanediyl)) dioctanoate |
| 2401 | 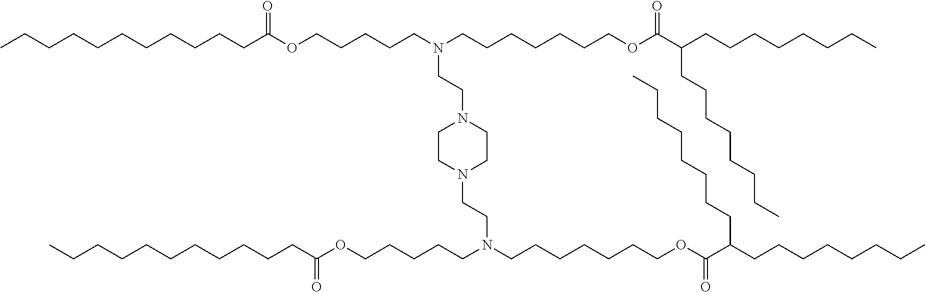 | ((piperazine-1,4- diylbis(ethane-2,1- diyl)) bis((7-((2- octyldecanoyl)oxy) heptyl)azanediyl)) bis(pentane-5,1-diyl) didodecanoate |
| 2403 | 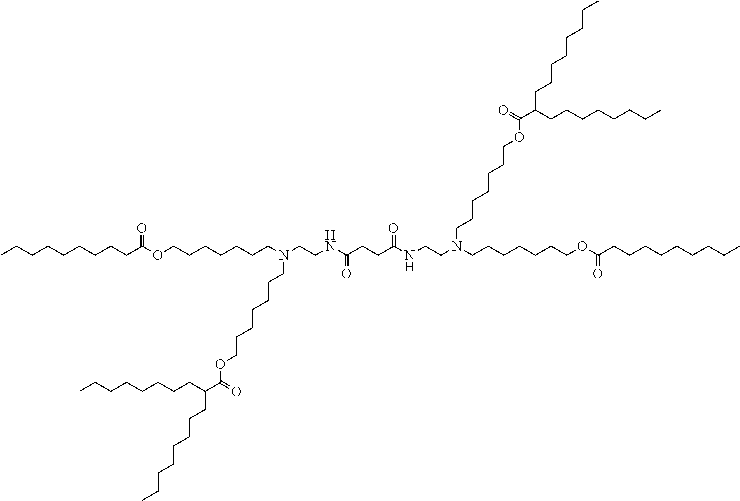 [SMILES: CCCCCCCCCC(=O)OCCCCCCCN(CCCCCCCOC(=O)C(CCCCCCCC)CCCCCCCC)CCNC(=O)CCC(=O)NCCN(CCCCCCCOC(=O)CCCCCCCCC)CCCCCCCOC(=O)C(CCCCCCCC)CCCCCCCC] | 8,19-bis(7- (decanoyloxy)heptyl)- 12,15-dioxo-8,11,16,19- tetraazahexacosane- 1,26-diyl bis(2- octyldecanoate) |
| 2404 | 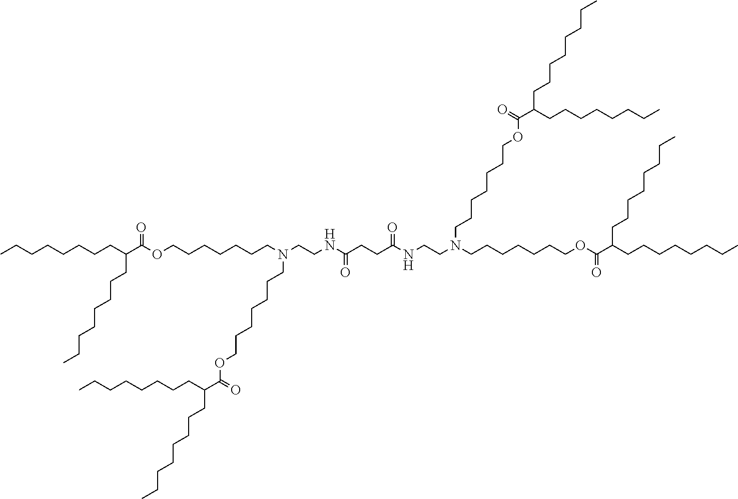 [SMILES: CCCCCCCCC(CCCCCCCC)C(=O)OCCCCCCCN(CCCCCCCOC(=O)C(CCCCCCCC)CCCCCCCC)CCNC(=O)CCC(=O)NCCN(CCCCCCCOC(=O)C(CCCCCCCC)CCCCCCCC)CCCCCCCOC(=O)C(CCCCCCCC)CCCCCCCC] | 7-[2-[4-[2-[bis[7-(2- octyldecanoyloxy)heptyl] amino]ethylamino]-4- oxo- butanoyl]amino]ethyl- [7-(2- octyldecanoyloxy)heptyl lamino]heptyl 2- octyldecanoate |
| 2405 | 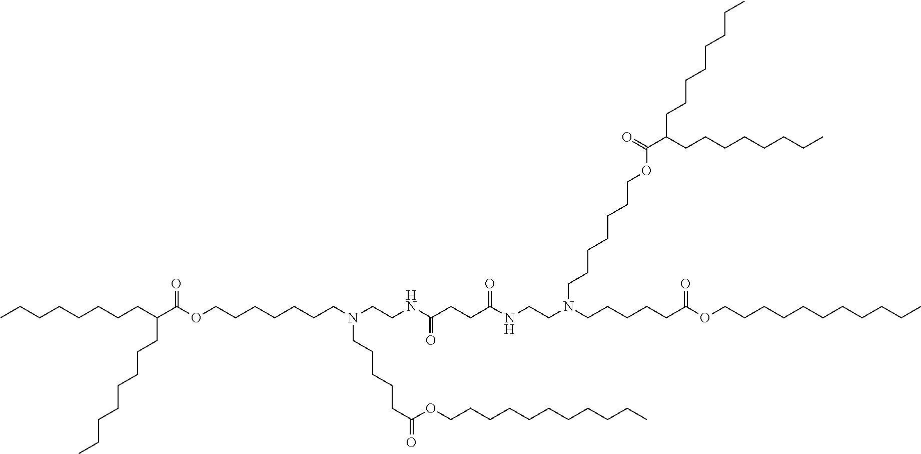 [SMILES: CCCCCCCCCCCOC(=O)CCCCCN(CCCCCCCOC(=O)C(CCCCCCCC)CCCCCCCC)CCNC(=O)CCC(=O)NCCN(CCCCCCCOC(=O)C(CCCCCCCC)CCCCCCCC)CCCCCC(=O)OCCCCCCCCCCC] | diundecyl 7,18-bis(7- ((2- octyldecanoyl)oxy) heptyl)-11,14-dioxo- 7,10,15,18- tetraazatetracosanedioate |
| 2421 | 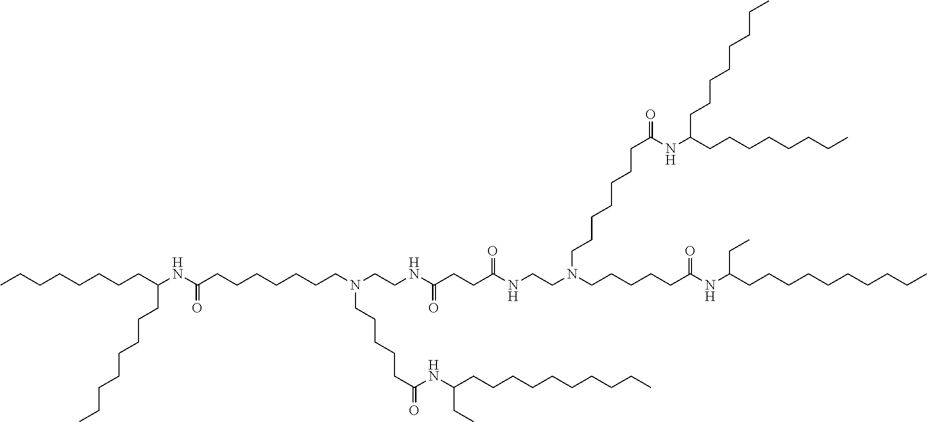 [SMILES: CCCCCCCCCCC(CC)NC(=O)CCCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCCC(=O)NC(CC)CCCCCCCCCC] | N1, N4-bis(2-((8- (heptadecan-9- ylamino)-8-oxooctyl)(6- oxo-6-(tridecan-3- ylamino)hexyl)amino) ethyl)succinamide |
| 2426 | 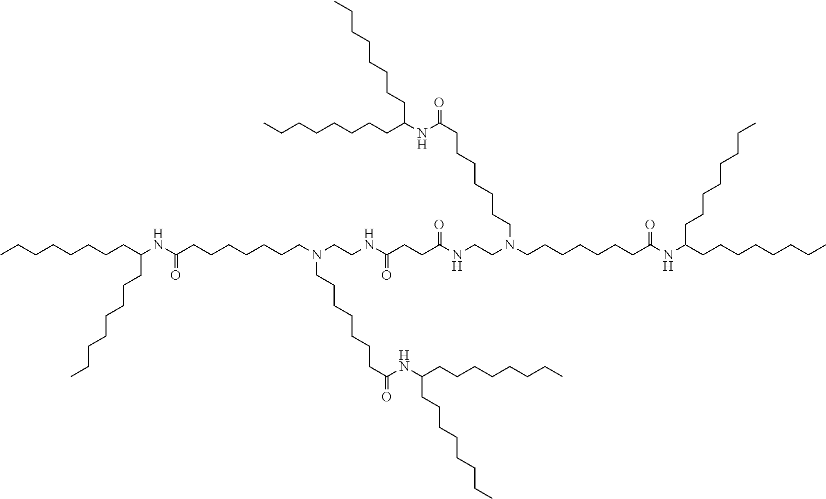 [SMILES: CCCCCCCCC(CCCCCCCC)NC(=O)CCCCCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCCCCC(=O)NC(CCCCCCCC)CCCCCCCC] | N1, N4-bis(2-(bis(8- (heptadecan-9- ylamino)-8- oxooctyl)amino)ethyl) succinamide |
| 2427 | 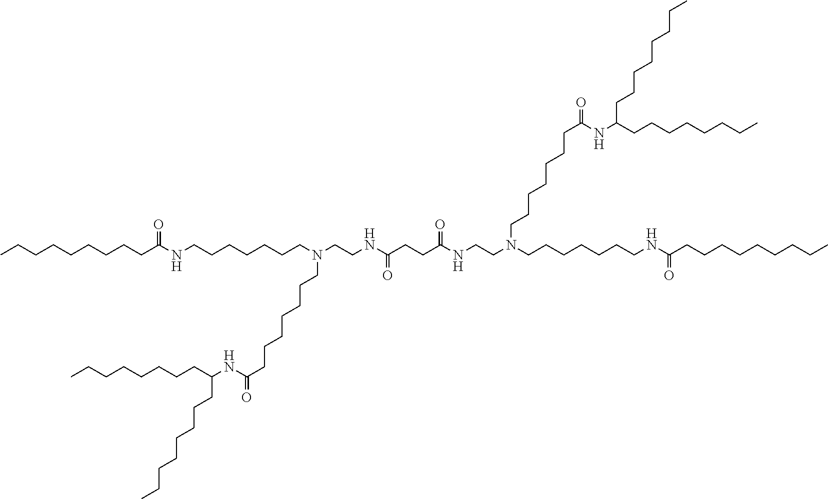 [SMILES: CCCCCCCCCC(=O)NCCCCCCCN(CCCCCCCC(=O)NC(CCCCCCCC)CCCCCCCC)CCNC(=O)CCC(=O)NCCN(CCCCCCCNC(=O)CCCCCCCCC)CCCCCCCC(=O)NC(CCCCCCCC)CCCCCCCC] | N1, N4-bis(2-((7- decanamidoheptyl)(8- (heptadecan-9- ylamino)-8- oxooctyl)amino)ethyl) succinamide |
| 2428 | 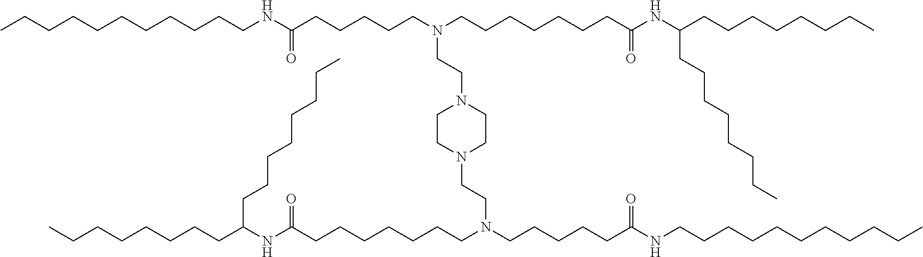 | 8,8′-((piperazine-1,4- diylbis(ethane-2,1- diyl))bis((6-oxo-6- (undecylamino)hexyl)az anediyl)) bis(N- (heptadecan-9- yl)octanamide) |
| | 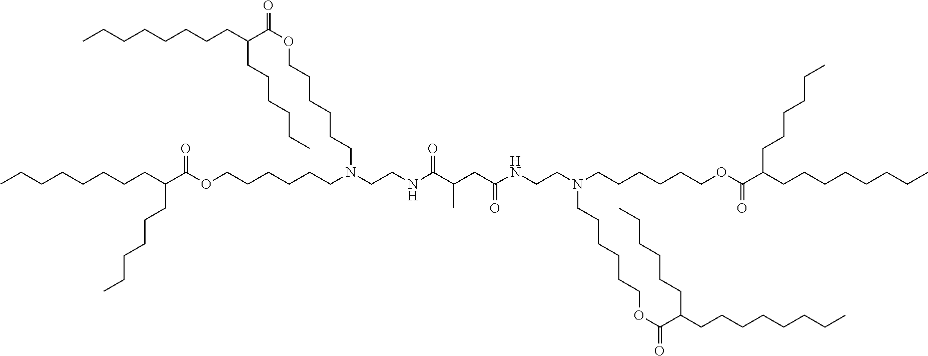 [SMILES: CCCCCCCCC(CCCCCC)C(=O)OCCCCCCN(CCCCCCOC(=O)C(CCCCCC)CCCCCCCC)CCNC(=O)CC(C)C(=O)NCCN(CCCCCCOC(=O)C(CCCCCC)CCCCCCCC)CCCCCCOC(=O)C(CCCCCC)CCCCCCCC] | 6-[2-[4-[2-[bis [6-(2- hexyldecanoyloxy)hexyl ]amino]ethylamino]-3- methyl-4-oxo- butanoyl]amino]ethyl- [6-(2- hexyldecanoyloxy) hexyl]amino]hexyl 2- hexyldecanoate |
| | 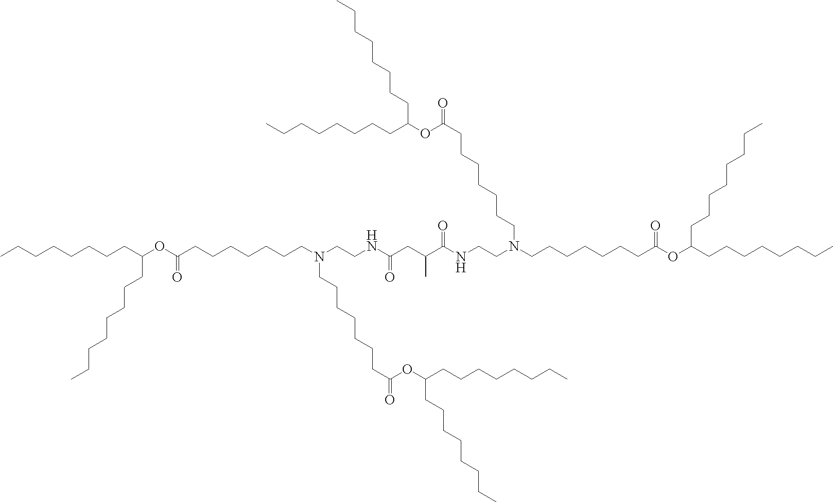 [SMILES: CCCCCCCCC(CCCCCCCC)OC(=O)CCCCCCCN(CCCCCCCC(=O)OC(CCCCCCCC)CCCCCCCC)CCNC(=O)CC(C)C(=O)NCCN(CCCCCCCC(=O)OC(CCCCCCCC)CCCCCCCC)CCCCCCCC(=O)OC(CCCCCCCC)CCCCCCCC] | di(heptadecan-9-yl) 9,20-bis(8- (heptadecan-9-yloxy)-8- oxooctyl)-14-methyl- 13,16-dioxo-9,12,17,20- tetraazaoctacosanedioate |
| | 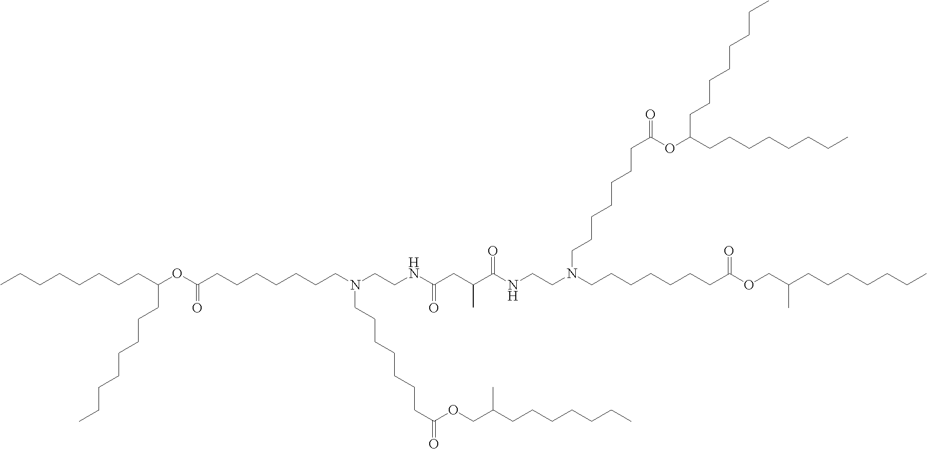 [SMILES: CCCCCCCCC(CCCCCCCC)OC(=O)CCCCCCCN(CCCCCCCC(=O)OCC(C)CCCCCCC)CCNC(=O)CC(C)C(=O)NCCN(CCCCCCCC(=O)OCC(C)CCCCCCC)CCCCCCCC(=O)OC(CCCCCCCC)CCCCCCCC] | di(heptadecan-9-yl) 14- methyl-9,20-bis(8-((2- methylnonyl)oxy)-8- oxooctyl)-13, 16-dioxo- 9,12,17,20- tetraazaoctacosanedioate |
| | 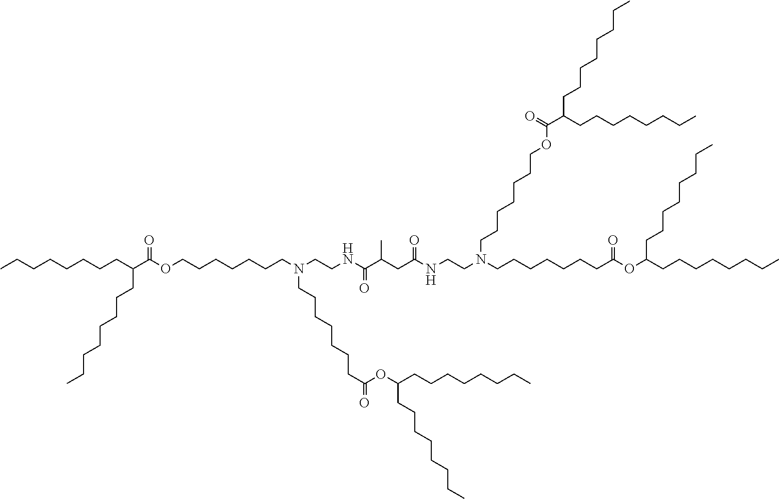 [SMILES: CCCCCCCCC(CCCCCCCC)OC(=O)CCCCCCCN(CCCCCCCOC(=O)C(CCCCCCCC)CCCCCCCC)CCNC(=O)CC(C)C(=O)NCCN(CCCCCCCOC(=O)C(CCCCCCCC)CCCCCCCC)CCCCCCCC(=O)OC(CCCCCCCC)CCCCCCCC] | di(heptadecan-9-yl) 14- methyl-9,20-bis(7-((2- octyldecanoyl)oxy)hept yl)-13,16-dioxo- 9,12,17,20- tetraazaoctacosanedioa te |
| | 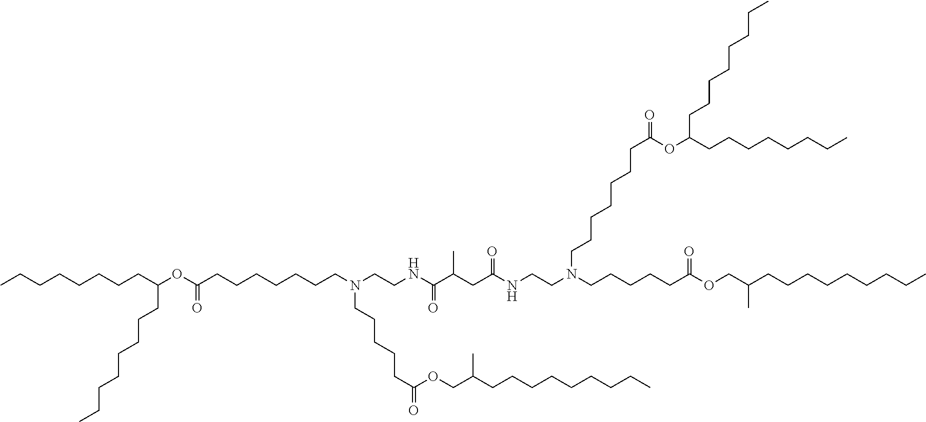 [SMILES: CCCCCCCCCC(C)COC(=O)CCCCCN(CCCCCCCC(=O)OC(CCCCCCCC)CCCCCCCC)CCNC(=O)CC(C)C(=O)NCCN(CCCCCCCC(=O)OC(CCCCCCCC)CCCCCCCC)CCCCCC(=O)OCC(C)CCCCCCCCC] | di(heptadecan-9-yl) 14- methyl-9,20-bis(6-((2- methylundecyl)oxy)-6- oxohexyl)-13, 16-dioxo- 9,12,17,20- tetraazaoctacosanedioa te |
| | 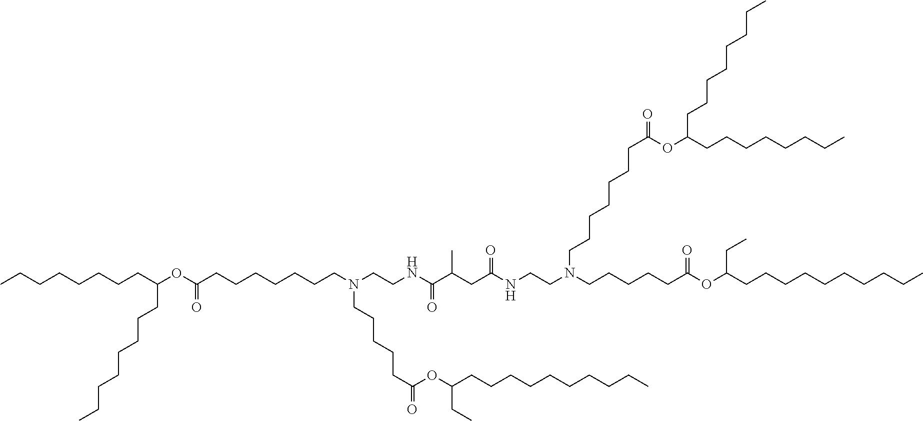 [SMILES: CCCCCCCCCCC(CC)OC(=O)CCCCCN(CCCCCCCC(=O)OC(CCCCCCCC)CCCCCCCC)CCNC(=O)CC(C)C(=O)NCCN(CCCCCCCC(=O)OC(CCCCCCCC)CCCCCCCC)CCCCCC(=O)OC(CC)CCCCCCCCCC] | di(heptadecan-9-yl) 14- methyl-13,16-dioxo- 9,20-bis(6-oxo-6- (tridecan-3- yloxy)hexyl)- 9,12,17,20- tetraazaoctacosanedioate |
| | 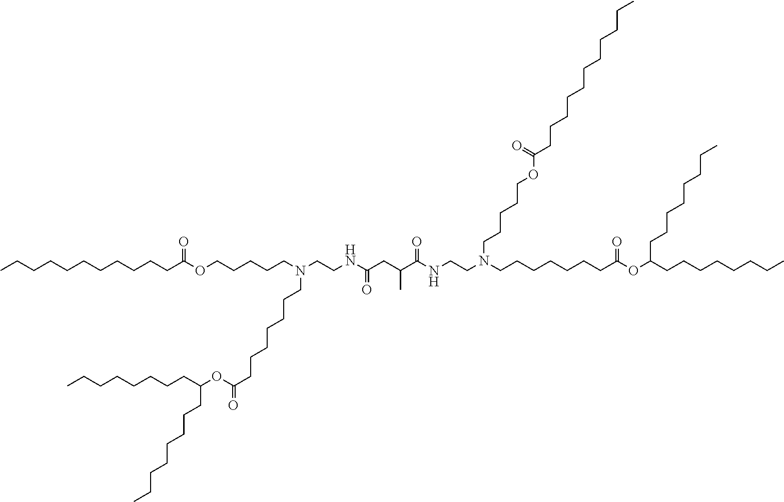 [SMILES: CCCCCCCCCCCC(=O)OCCCCCN(CCCCCCCC(=O)OC(CCCCCCCC)CCCCCCCC)CCNC(=O)CC(C)C(=O)NCCN(CCCCCCCC(=O)OC(CCCCCCCC)CCCCCCCC)CCCCCOC(=O)CCCCCCCCCCC] | di(heptadecan-9-yl) 9,20-bis(5- (dodecanoyloxy)pentyl)- 14-methyl-13, 16-dioxo- 9,12,17,20- tetraazaoctacosanedioate |
| | 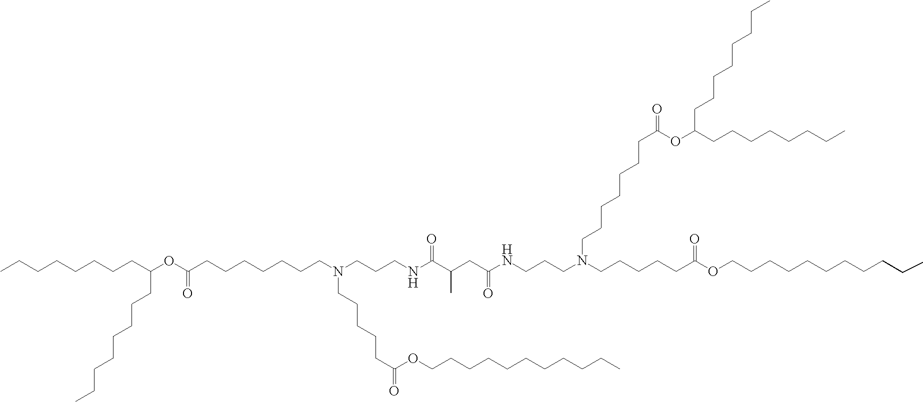 [SMILES: CCCCCCCCCCCOC(=O)CCCCCN(CCCCCCCC(=O)OC(CCCCCCCC)CCCCCCCC)CCCNC(=O)CC(C)C(=O)NCCCN(CCCCCCCC(=O)OC(CCCCCCCC)CCCCCCCC)CCCCCC(=O)OCCCCCCCCCCC] | di(heptadecan-9-yl) 15- methyl-14, 17-dioxo- 9,22-bis(6-oxo-6- (undecyloxy)hexyl)- 9,13,18,22- tetraazatriacontanedioate |
| | 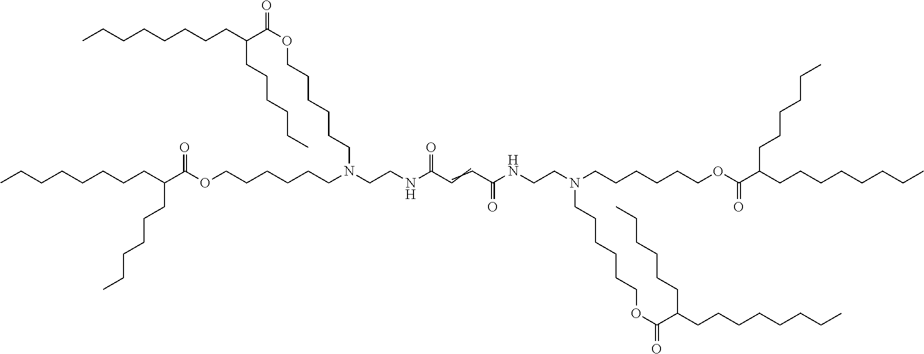 [SMILES: CCCCCCCCC(CCCCCC)C(=O)OCCCCCCN(CCCCCCOC(=O)C(CCCCCC)CCCCCCCC)CCNC(=O)C=CC(=O)NCCN(CCCCCCOC(=O)C(CCCCCC)CCCCCCCC)CCCCCCOC(=O)C(CCCCCC)CCCCCCCC] | 6-[2-[4-[2-[bis[6-(2- hexyldecanoyloxy)hexyl] amino]ethylamino]-4- oxo-but-2- enoyl]amino]ethyl-[6-(2- hexyldecanoyloxy)hexyl] amino]hexyl 2- hexyldecanoate |
| | 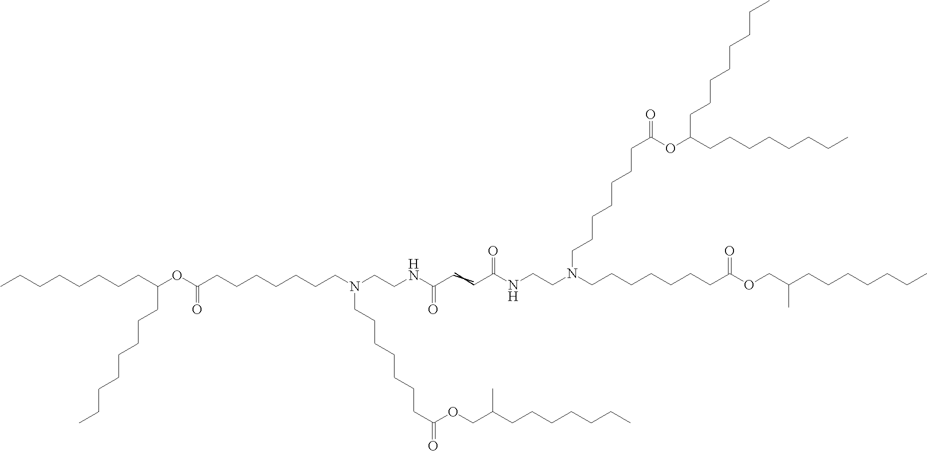 [SMILES: CCCCCCCCC(CCCCCCCC)OC(=O)CCCCCCCN(CCCCCCCC(=O)OCC(C)CCCCCCC)CCNC(=O)C=CC(=O)NCCN(CCCCCCCC(=O)OCC(C)CCCCCCC)CCCCCCCC(=O)OC(CCCCCCCC)CCCCCCCC] | di(heptadecan-9-yl) 9,20-bis(8-((2- methylnonyl)oxy)-8- oxooctyl)-13, 16-dioxo- 9,12,17,20- tetraazaoctacos-14- enedioate |
| | 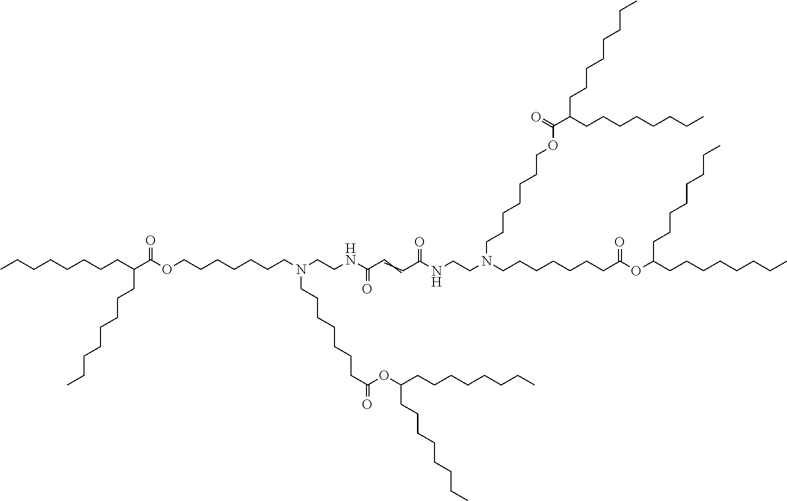 [SMILES: CCCCCCCCC(CCCCCCCC)OC(=O)CCCCCCCN(CCCCCCCOC(=O)C(CCCCCCCC)CCCCCCCC)CCNC(=O)C=CC(=O)NCCN(CCCCCCCOC(=O)C(CCCCCCCC)CCCCCCCC)CCCCCCCC(=O)OC(CCCCCCCC)CCCCCCCC] | di(heptadecan-9-yl) 9,20-bis (7-((2- octyldecanoyl)oxy)hept yl)-13,16-dioxo- 9,12,17,20- tetraazaoctacos-14- enedioate |
| | 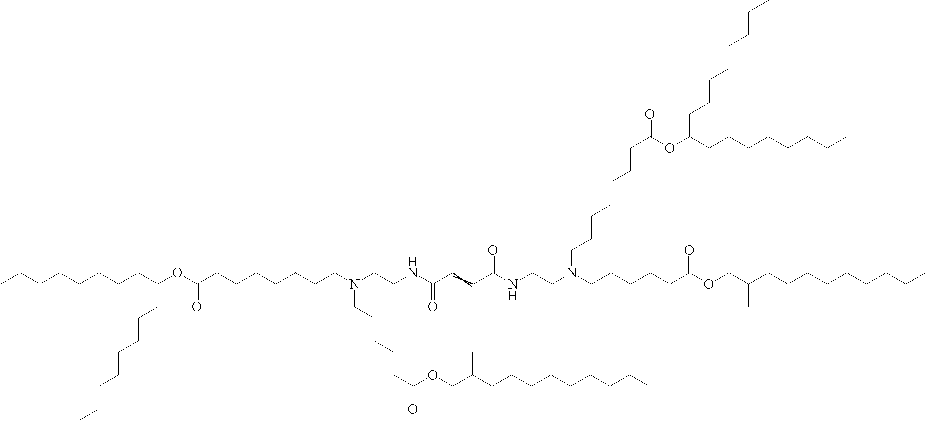 [SMILES: CCCCCCCCCC(C)COC(=O)CCCCCN(CCCCCCCC(=O)OC(CCCCCCCC)CCCCCCCC)CCNC(=O)C=CC(=O)NCCN(CCCCCCCC(=O)OC(CCCCCCCC)CCCCCCCC)CCCCCC(=O)OCC(C)CCCCCCCCC] | di(heptadecan-9-yl) 9,20-bis (6-((2- methylundecyl)oxy)-6- oxohexyl)-13, 16-dioxo- 9,12,17,20- tetraazaoctacos-14- enedioate |
| | 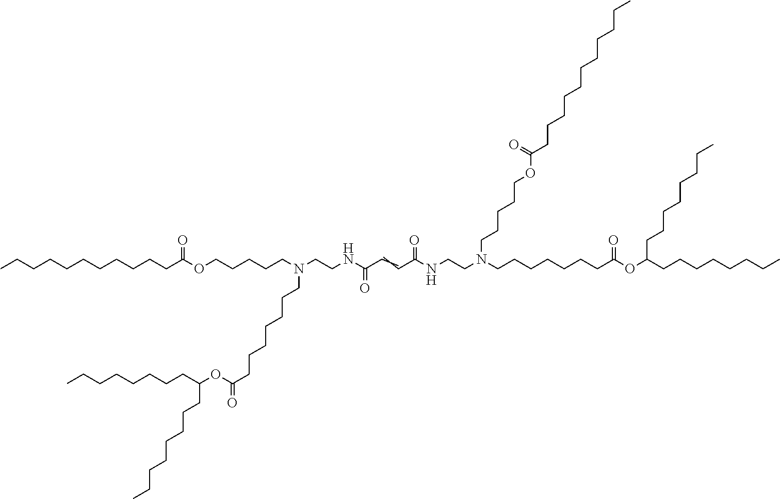 [SMILES: CCCCCCCCCCCC(=O)OCCCCCN(CCCCCCCC(=O)OC(CCCCCCCC)CCCCCCCC)CCNC(=O)C=CC(=O)NCCN(CCCCCCCC(=O)OC(CCCCCCCC)CCCCCCCC)CCCCCOC(=O)CCCCCCCCCCC] | di(heptadecan-9-yl) 9,20-bis(5- (dodecanoyloxy)pentyl)- 13,16-dioxo- 9,12,17,20- tetraazaoctacos-14- enedioate |
| | 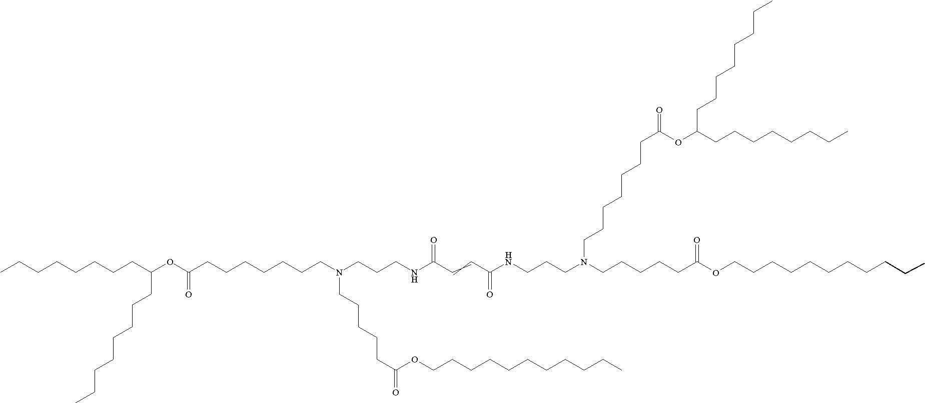 [SMILES: CCCCCCCCCCCOC(=O)CCCCCN(CCCCCCCC(=O)OC(CCCCCCCC)CCCCCCCC)CCCNC(=O)C=CC(=O)NCCCN(CCCCCCCC(=O)OC(CCCCCCCC)CCCCCCCC)CCCCCC(=O)OCCCCCCCCCCC] | di(heptadecan-9-yl) 14,17-dioxo-9,22-bis(6- oxo-6- (undecyloxy)hexyl)- 9,13,18,22- tetraazatriacont-15- enedioate |
| | 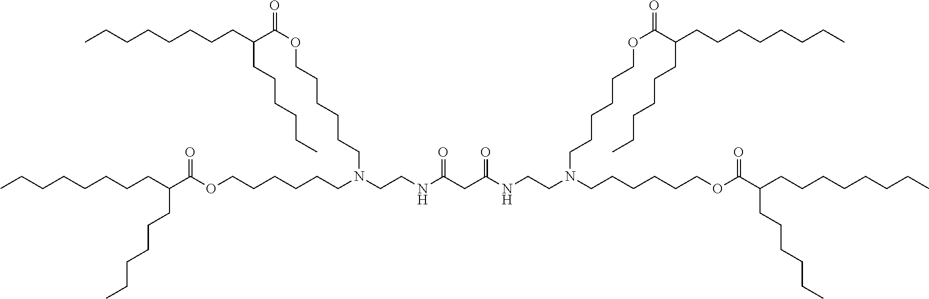 [SMILES: CCCCCCCCC(CCCCCC)C(=O)OCCCCCCN(CCCCCCOC(=O)C(CCCCCC)CCCCCCCC)CCNC(=O)CC(=O)NCCN(CCCCCCOC(=O)C(CCCCCC)CCCCCCCC)CCCCCCOC(=O)C(CCCCCC)CCCCCCCC] | 6-[2-[4-[2-[bis[6-(2- hexyldecanoyloxy)hexyl] amino]ethylamino]-4- oxo-but-2- enoyl]amino]ethyl-[6-(2- hexyldecanoyloxy)hexyl] amino]hexyl 2- hexyldecanoate |
| | 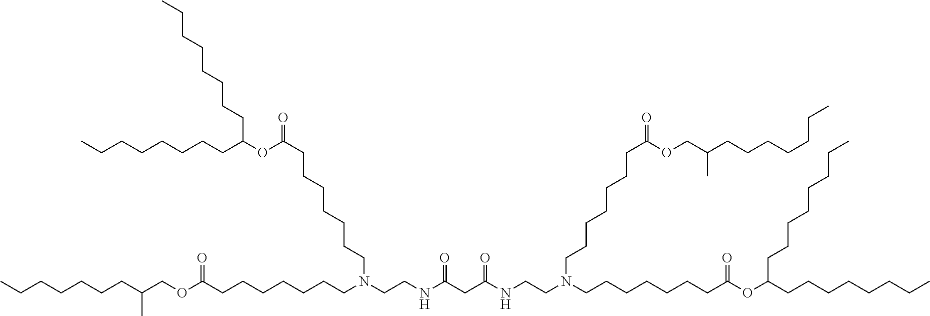 [SMILES: CCCCCCCCC(CCCCCCCC)OC(=O)CCCCCCCN(CCCCCCCC(=O)OCC(C)CCCCCCC)CCNC(=O)CC(=O)NCCN(CCCCCCCC(=O)OCC(C)CCCCCCC)CCCCCCCC(=O)OC(CCCCCCCC)CCCCCCCC] | di(heptadecan-9-yl) 9,19-bis(8-((2- methylnonyl)oxy)-8- oxooctyl)-13,15-dioxo- 9,12,16,19- tetraazaheptacosanedioate |
| | 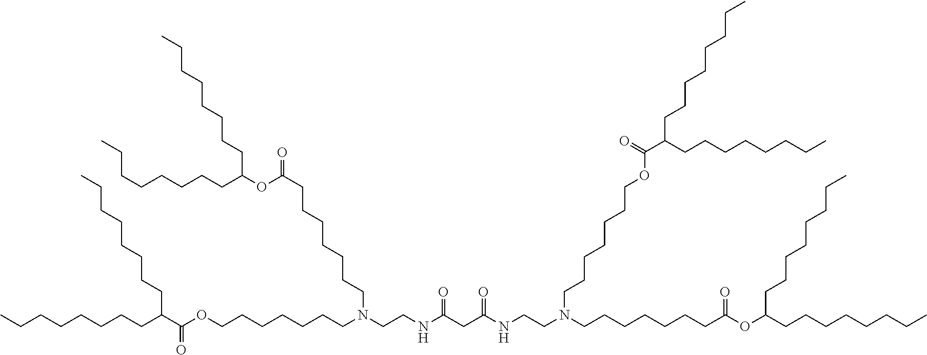 [SMILES: CCCCCCCCC(CCCCCCCC)OC(=O)CCCCCCCN(CCCCCCCOC(=O)C(CCCCCCCC)CCCCCCCC)CCNC(=O)CC(=O)NCCN(CCCCCCCOC(=O)C(CCCCCCCC)CCCCCCCC)CCCCCCCC(=O)OC(CCCCCCCC)CCCCCCCC] | di(heptadecan-9-yl) 9,19-bis(7-((2- octyldecanoyl)oxy)hept yl)-13,15-dioxo- 9,12,16,19- tetraazaheptacosanedi oate |
| | 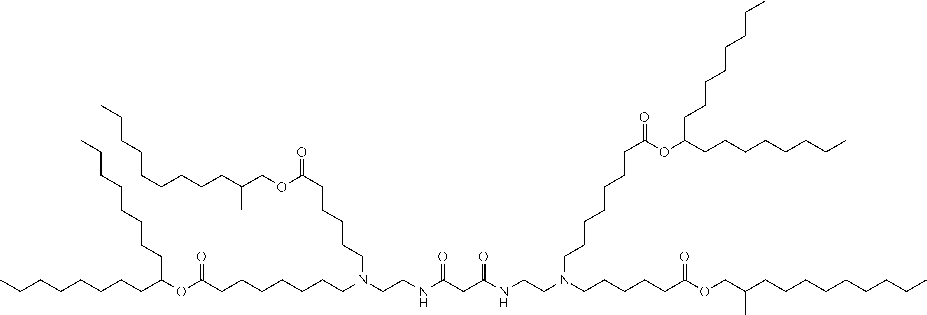 [SMILES: CCCCCCCCCC(C)COC(=O)CCCCCN(CCCCCCCC(=O)OC(CCCCCCCC)CCCCCCCC)CCNC(=O)CC(=O)NCCN(CCCCCCCC(=O)OC(CCCCCCCC)CCCCCCCC)CCCCCC(=O)OCC(C)CCCCCCCCC] | di(heptadecan-9-yl) 9,19-bis(6-((2- methylundecyl)oxy)-6- oxohexyl)-13, 15-dioxo- 9,12,16,19- tetraazaheptacosanedioate |
| | 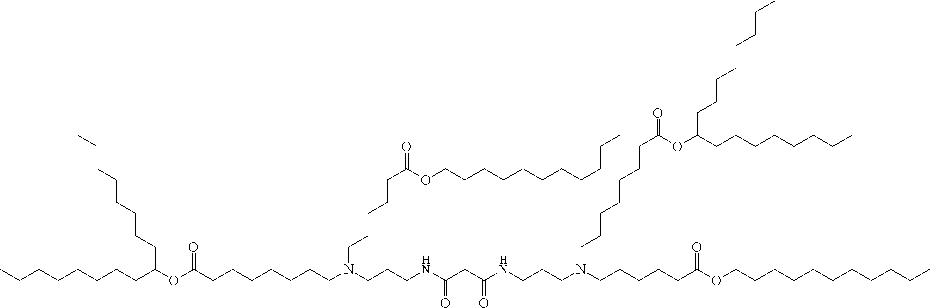 [SMILES: CCCCCCCCCCCOC(=O)CCCCCN(CCCCCCCC(=O)OC(CCCCCCCC)CCCCCCCC)CCCNC(=O)CC(=O)NCCCN(CCCCCCCC(=O)OC(CCCCCCCC)CCCCCCCC)CCCCCC(=O)OCCCCCCCCCCC] | di(heptadecan-9-yl) 14,16-dioxo-9,21-bis(6- oxo-6- (undecyloxy)hexyl)- 9,13,17,21- tetraazanonacosanedioate |
| | 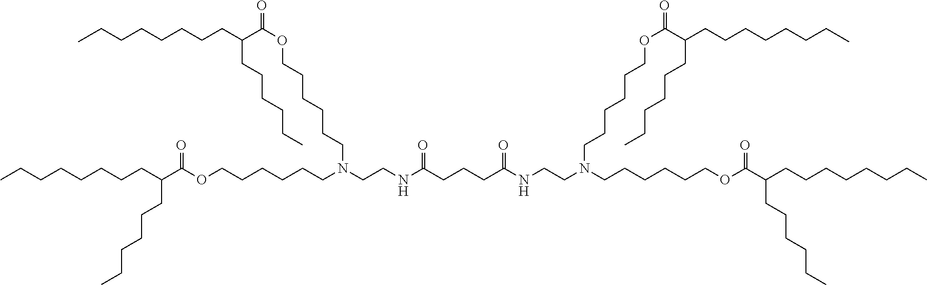 [SMILES: CCCCCCCCC(CCCCCC)C(=O)OCCCCCCN(CCCCCCOC(=O)C(CCCCCC)CCCCCCCC)CCNC(=O)CCCC(=O)NCCN(CCCCCCOC(=O)C(CCCCCC)CCCCCCCC)CCCCCCOC(=O)C(CCCCCC)CCCCCCCC] | 6-[2-[5-[2-[bis[6-(2- hexyldecanoyloxy)hexyl] amino]ethylamino]-5- oxo- pentanoyl]amino]ethyl- [6-(2- hexyldecanoyloxy)hexyl] amino]hexyl 2- hexyldecanoate |
| | 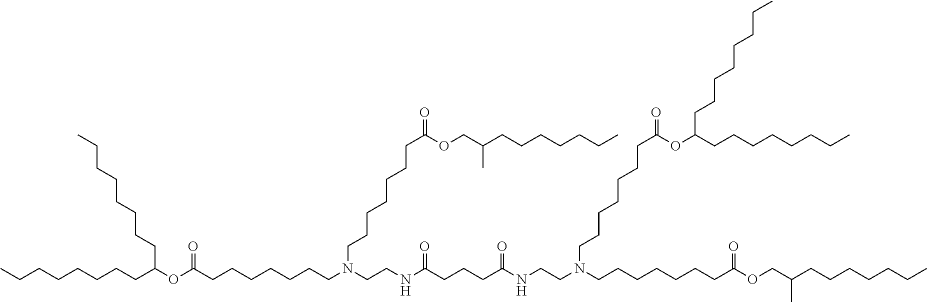 [SMILES: CCCCCCCCC(CCCCCCCC)OC(=O)CCCCCCCN(CCCCCCCC(=O)OCC(C)CCCCCCC)CCNC(=O)CCCC(=O)NCCN(CCCCCCCC(=O)OCC(C)CCCCCCC)CCCCCCCC(=O)OC(CCCCCCCC)CCCCCCCC] | di(heptadecan-9-yl) 9,21-bis(8-((2- methylnonyl)oxy)-8- oxooctyl)-13, 17-dioxo- 9,12,18,21- tetraazanonacosanedioate |
| | 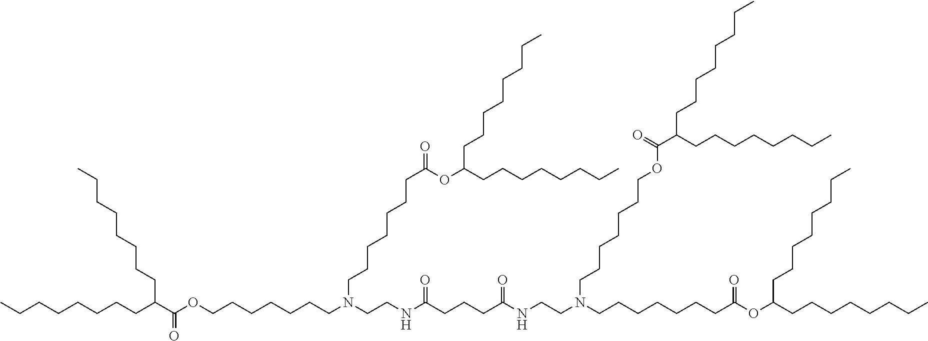 [SMILES: CCCCCCCCC(CCCCCCCC)OC(=O)CCCCCCCN(CCCCCCCOC(=O)C(CCCCCCCC)CCCCCCCC)CCNC(=O)CCCC(=O)NCCN(CCCCCCCOC(=O)C(CCCCCCCC)CCCCCCCC)CCCCCCCC(=O)OC(CCCCCCCC)CCCCCCCC] | di(heptadecan-9-yl) 9,21-bis(7-((2- octyldecanoyl)oxy)hept yl)-13,17-dioxo- 9,12,18,21- tetraazanonacosanedio ate |
| | 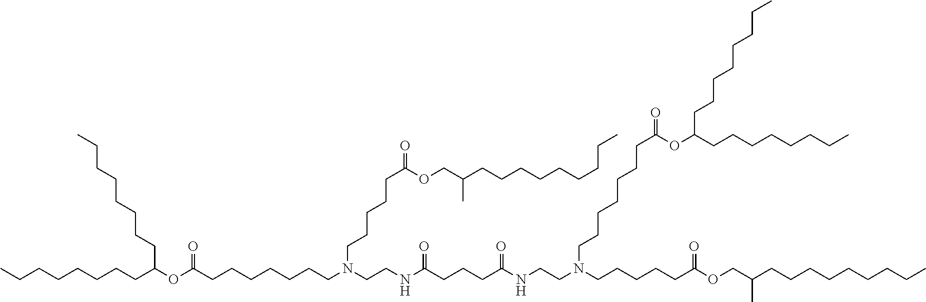 [SMILES: CCCCCCCCCC(C)COC(=O)CCCCCN(CCCCCCCC(=O)OC(CCCCCCCC)CCCCCCCC)CCNC(=O)CCCC(=O)NCCN(CCCCCCCC(=O)OC(CCCCCCCC)CCCCCCCC)CCCCCC(=O)OCC(C)CCCCCCCCC] | di(heptadecan-9-yl) 9,21-bis(6-((2- methylundecyl)oxy)-6- oxohexyl)-13,17-dioxo- 9,12,18,21- tetraazanonacosanedio ate |
| | 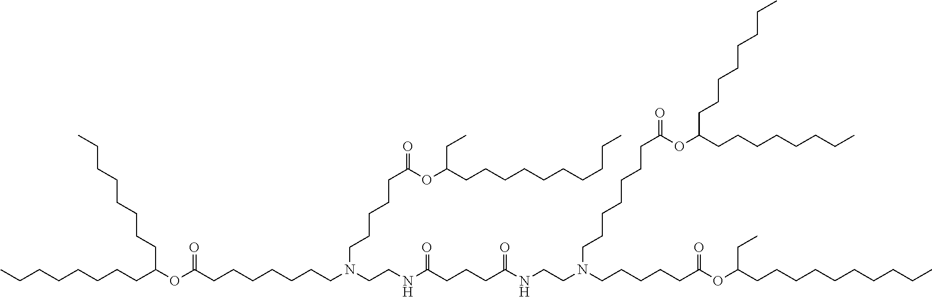 [SMILES: CCCCCCCCCCC(CC)OC(=O)CCCCCN(CCCCCCCC(=O)OC(CCCCCCCC)CCCCCCCC)CCNC(=O)CCCC(=O)NCCN(CCCCCCCC(=O)OC(CCCCCCCC)CCCCCCCC)CCCCCC(=O)OC(CC)CCCCCCCCCC] | di(heptadecan-9-yl) 13,17-dioxo-9,21-bis(6- oxo-6-(tridecan-3- yloxy)hexyl)- 9,12,18,21- tetraazanonacosanedio ate |
| | 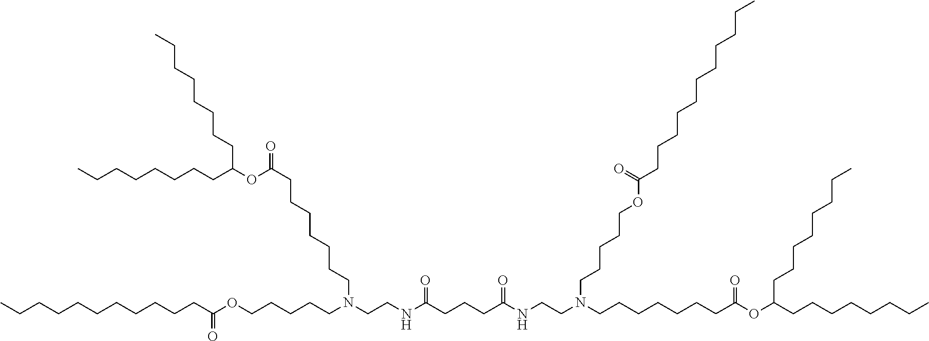 | di(heptadecan-9-yl) 9,21-bis(5- (dodecanoyloxy)pentyl)- 13,17-dioxo- 9,12,18,21- tetraazanonacosanedioate |
| | 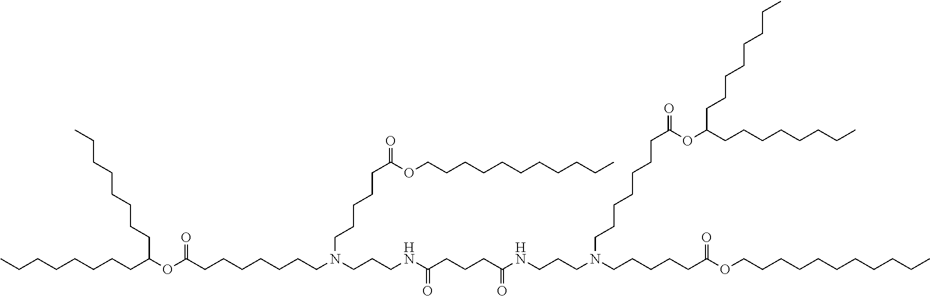 [SMILES: CCCCCCCCCCCOC(=O)CCCCCN(CCCCCCCC(=O)OC(CCCCCCCC)CCCCCCCC)CCCNC(=O)CCCC(=O)NCCCN(CCCCCCCC(=O)OC(CCCCCCCC)CCCCCCCC)CCCCCC(=O)OCCCCCCCCCCC] | di(heptadecan-9-yl) 14,18-dioxo-9,23-bis(6- oxo-6- (undecyloxy)hexyl)- 9,13,19,23- tetraazahentriacontane dioate |
| | 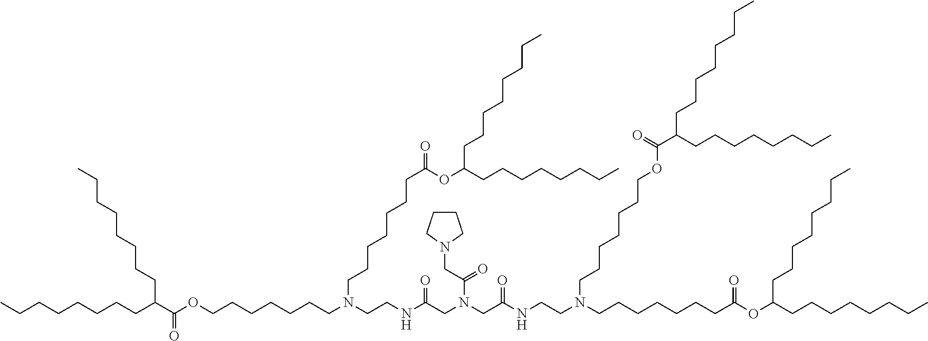 [SMILES: CCCCCCCCC(CCCCCCCC)OC(=O)CCCCCCCN(CCCCCCCOC(=O)C(CCCCCCCC)CCCCCCCC)CCNC(=O)CN(CC(=O)NCCN(CCCCCCCOC(=O)C(CCCCCCCC)CCCCCCCC)CCCCCCCC(=O)OC(CCCCCCCC)CCCCCCCC)C(=O)CN1CCCC1] | 6-[2-[2-[2-[2-[bis [6-(2- hexyldecanoyloxy)hexyl lamino]ethylamino]-2- oxo-ethyl]-(2-pyrrolidin-1- ylacetyl)amino]acetyl] amino]ethyl-[6-(2- hexyldecanoyloxy)hexyl] amino]hexyl 2- hexyldecanoate |
| | 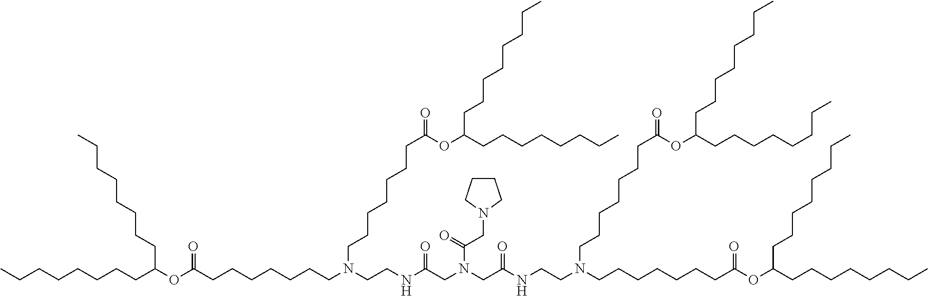 [SMILES: CCCCCCCCC(CCCCCCCC)OC(=O)CCCCCCCN(CCCCCCCC(=O)OC(CCCCCCCC)CCCCCCCC)CCNC(=O)CN(CC(=O)NCCN(CCCCCCCC(=O)OC(CCCCCCCC)CCCCCCCC)CCCCCCCC(=O)OC(CCCCCCCC)CCCCCCCC)C(=O)CN1CCCC1] | di(heptadecan-9-yl) 9,21-bis(8- (heptadecan-9-yloxy)-8- oxooctyl)-13,17-dioxo- 15-(2-(pyrrolidin-1- yl)acetyl)- 9,12,15,18,21- pentaazanonacosanedioate |
| | 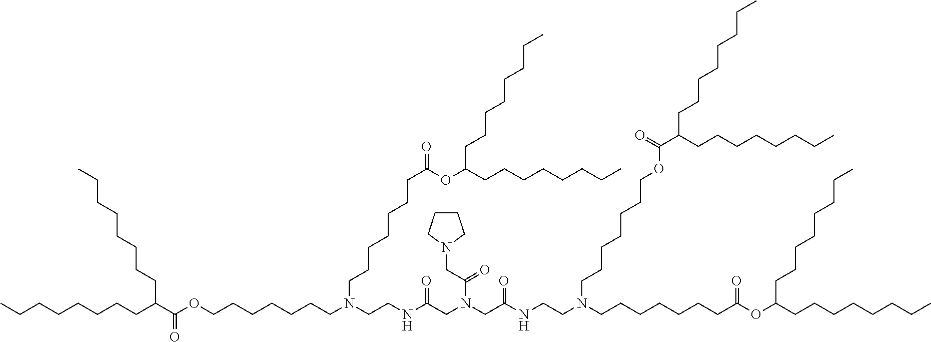 [SMILES: CCCCCCCCC(CCCCCCCC)OC(=O)CCCCCCCN(CCCCCCCOC(=O)C(CCCCCCCC)CCCCCCCC)CCNC(=O)CN(CC(=O)NCCN(CCCCCCCOC(=O)C(CCCCCCCC)CCCCCCCC)CCCCCCCC(=O)OC(CCCCCCCC)CCCCCCCC)C(=O)CN1CCCC1] | di(heptadecan-9-yl) 9,21-bis(8-((2- methylnonyl)oxy)-8- oxooctyl)-13, 17-dioxo- 15-(2-(pyrrolidin-1- yl)acetyl)- 9,12,15,18,21- pentaazanonacosanedioate |
| | 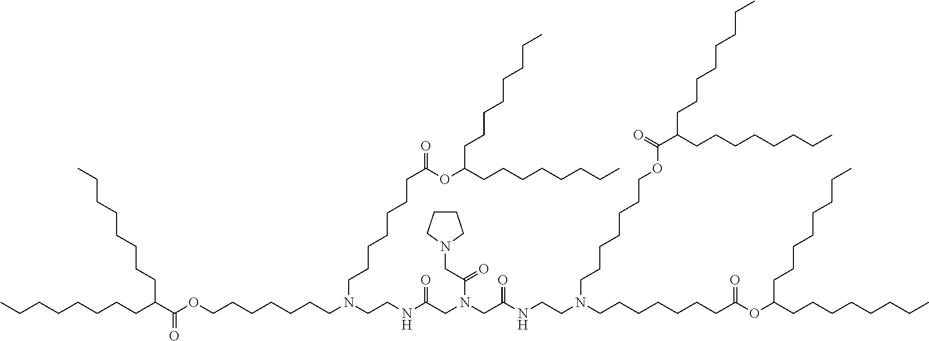 [SMILES: CCCCCCCCC(CCCCCCCC)OC(=O)CCCCCCCN(CCCCCCCOC(=O)C(CCCCCCCC)CCCCCCCC)CCNC(=O)CN(CC(=O)NCCN(CCCCCCCOC(=O)C(CCCCCCCC)CCCCCCCC)CCCCCCCC(=O)OC(CCCCCCCC)CCCCCCCC)C(=O)CN1CCCC1] | di(heptadecan-9-yl) 9,21-bis(7-((2- octyldecanoyl)oxy) heptyl)-13,17-dioxo-15- (2-(pyrrolidin-1-yl)acetyl)- 9,12,15,18,21- pentaazanonacosanedioate |
| | 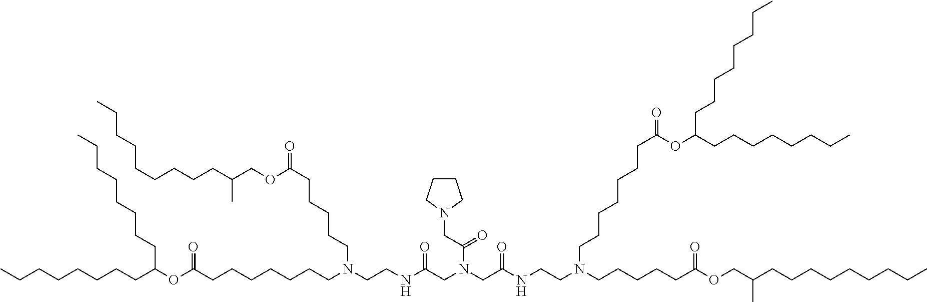 [SMILES: CCCCCCCCCC(C)COC(=O)CCCCCN(CCCCCCCC(=O)OC(CCCCCCCC)CCCCCCCC)CCNC(=O)CN(CC(=O)NCCN(CCCCCCCC(=O)OC(CCCCCCCC)CCCCCCCC)CCCCCC(=O)OCC(C)CCCCCCCCC)C(=O)CN1CCCC1] | di(heptadecan-9-yl) 9,21-bis(6-((2- methylundecyl)oxy)-6- oxohexyl)-13,17-dioxo- 15-(2-(pyrrolidin-1- yl)acetyl)- 9,12,15,18,21- pentaazanonacosanedioate |
| | 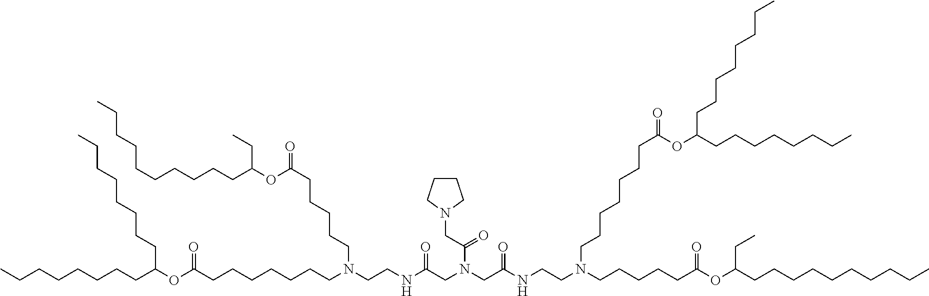 [SMILES: CCCCCCCCCCC(CC)OC(=O)CCCCCN(CCCCCCCC(=O)OC(CCCCCCCC)CCCCCCCC)CCNC(=O)CN(CC(=O)NCCN(CCCCCCCC(=O)OC(CCCCCCCC)CCCCCCCC)CCCCCC(=O)OC(CC)CCCCCCCCCC)C(=O)CN1CCCC1] | di(heptadecan-9-yl) 13,17-dioxo-9,21-bis(6- oxo-6-(tridecan-3- yloxy)hexyl)-15-(2- (pyrrolidin-1-yl)acetyl)- 9,12,15,18,21- pentaazanonacosanedioate |
| | 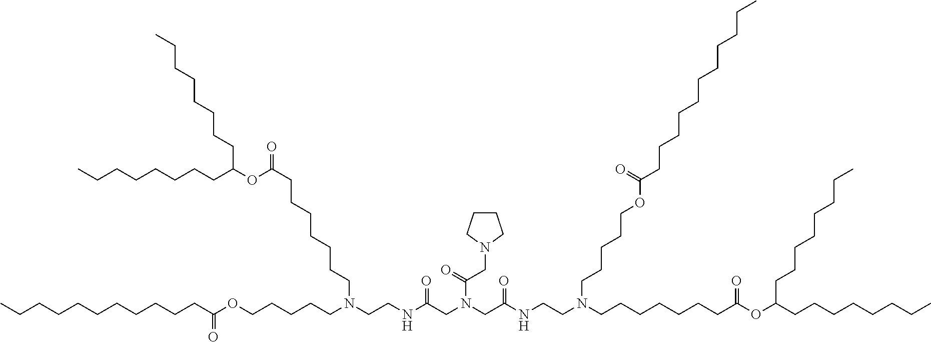 | di(heptadecan-9-yl) 9,21-bis(5- (dodecanoyloxy)pentyl)- 13,17-dioxo-15-(2- (pyrrolidin-1-yl)acetyl)- 9,12,15,18,21- pentaazanonacosanedioate |
| | 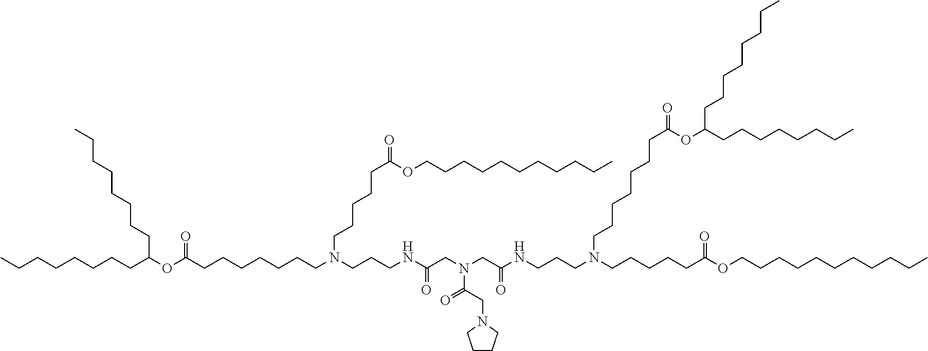 [SMILES: CCCCCCCCCCCOC(=O)CCCCCN(CCCCCCCC(=O)OC(CCCCCCCC)CCCCCCCC)CCCNC(=O)CN(CC(=O)NCCCN(CCCCCCCC(=O)OC(CCCCCCCC)CCCCCCCC)CCCCCC(=O)OCCCCCCCCCCC)C(=O)CN1CCCC1] | di(heptadecan-9-yl) 14,18-dioxo-9,23-bis(6- oxo-6- (undecyloxy)hexyl)-16- (2-(pyrrolidin-1- yl)acetyl)- 9,13,16,19,23- pentaazahentria- contanedioate |
| | 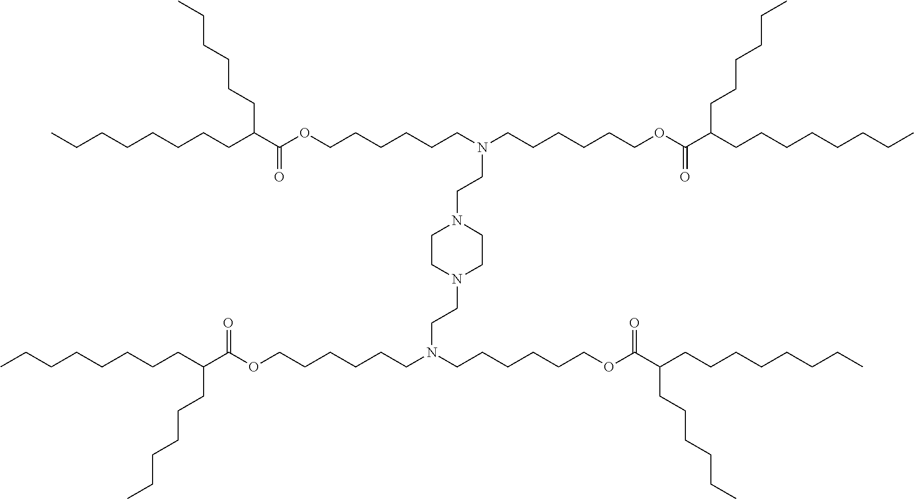 | ((piperazine-1,4- diylbis(ethane-2,1- diyl)) bis(azanetriyl)) tetrakis(hexane-6,1-diyl) tetrakis(2- hexyldecanoate) |
| | 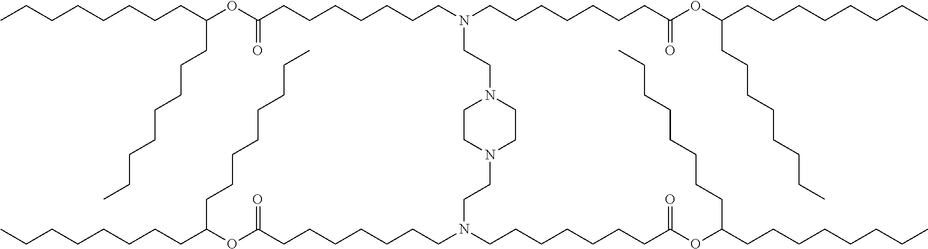 [SMILES: CCCCCCCCC(CCCCCCCC)OC(=O)CCCCCCCN(CCCCCCCC(=O)OC(CCCCCCCC)CCCCCCCC)CCN1CCN(CCN(CCCCCCCC(=O)OC(CCCCCCCC)CCCCCCCC)CCCCCCCC(=O)OC(CCCCCCCC)CCCCCCCC)CC1] | tetra(heptadecan-9-yl) 8,8′,8″,8″-((piperazine- 1,4-diylbis(ethane-2,1- diyl))bis(azanetriyl))tetr aoctanoate |
| | 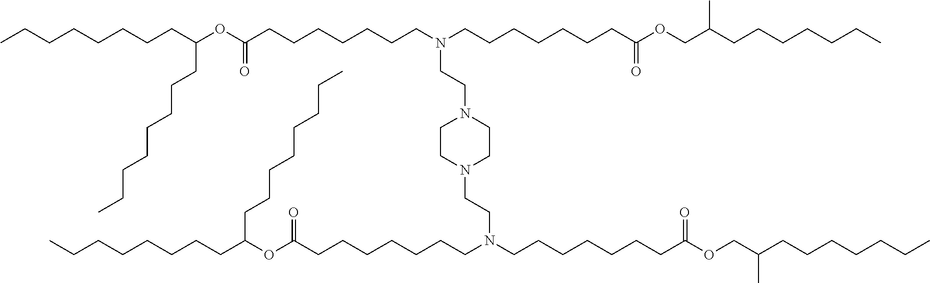 | di(heptadecan-9-yl) 8,8′-((piperazine-1,4- diylbis(ethane-2,1- diyl)) bis((8-((2- methylnonyl)oxy)-8- oxooctyl)azanediyl)) dioctanoate |
| | 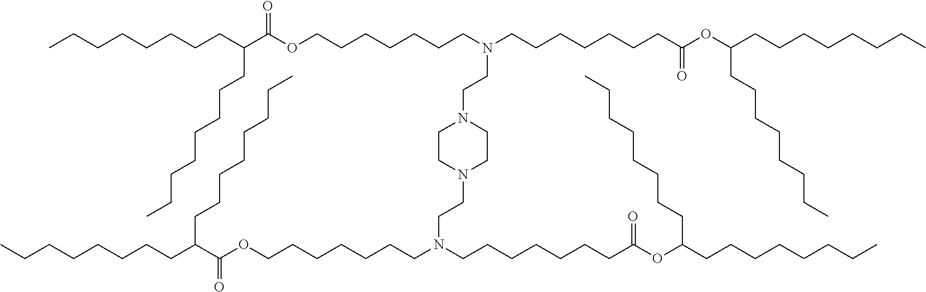 | ((piperazine-1,4- diylbis(ethane-2,1- diyl)) bis((8- (heptadecan-9-yloxy)-8- oxooctyl)azanediyl))bis (heptane-7,1-diyl) bis(2- octyldecanoate) |
| | 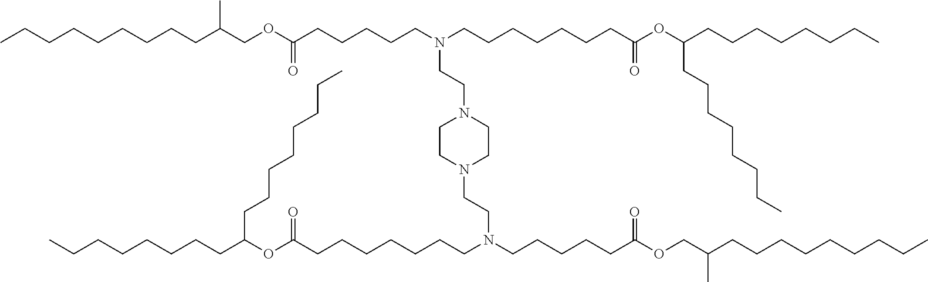 | di(heptadecan-9-yl) 8,8′-((piperazine-1,4- diylbis(ethane-2,1- diyl)) bis ((6-((2- methylundecyl)oxy)-6- oxohexyl)azanediyl)) dioctanoate |
| | 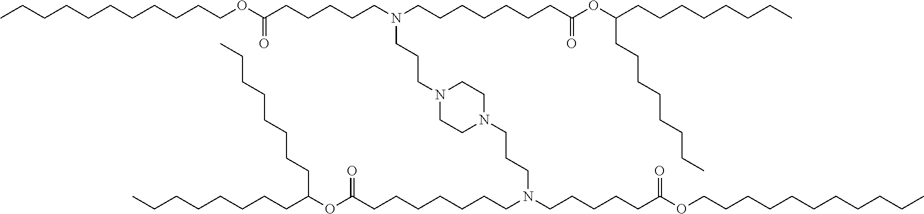 [SMILES: CCCCCCCCCCCOC(=O)CCCCCN(CCCCCCCC(=O)OC(CCCCCCCC)CCCCCCCC)CCCN1CCN(CCCN(CCCCCCCC(=O)OC(CCCCCCCC)CCCCCCCC)CCCCCC(=O)OCCCCCCCCCCC)CC1] | di(heptadecan-9-yl) 8,8′-((piperazine-1,4- diylbis(propane-3,1- diyl))bis((6-oxo-6- (undecyloxy)hexyl) azanediyl))dioctanoate |

### Ionizable Lipid Compounds ii)

In some embodiments, the ionizable lipid is represented by the following formula II:

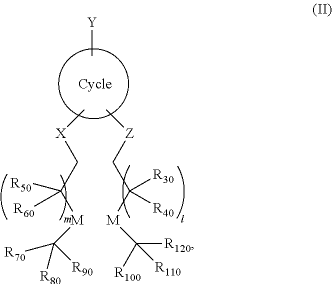

a pharmaceutically acceptable salt thereof, or a stereoisomer of any of the foregoing, wherein:

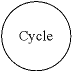

is cyclic or heterocyclic moiety;
Y is alkyl, hydroxy, hydroxyalkyl or

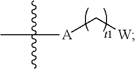

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each of X and Z is independently absent, —O—, —CO—, —N(R⁷)—, —O-alkylene-; -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, hydroxyalkyl, or aminoalkyl;
each M is independently a biodegradable moiety;
each of R₃₀, R₄₀, R₅₀, R₆₀, R₇₀, R₈₀, R₉₀, R₁₀₀, R₁₁₀, and R₁₂₀ is independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
each of l and m is an integer from 1 to 10;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl.

In some embodiments, Y is hydroxyl or

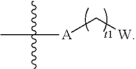

In some embodiments,

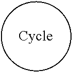

is selected from pyrrolidine, piperidine, piperazine, cyclohexane, cyclopentane, tetrahydrofuran, tetrahydropyran, morpholine, and dioxane. In some embodiments,

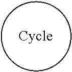

is selected from the group consisting of

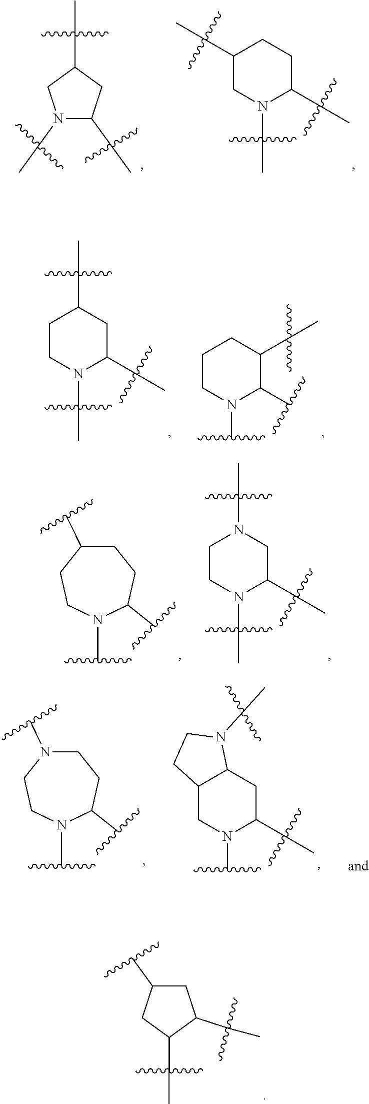

In some embodiments, the ionizable lipid is represented by formula:

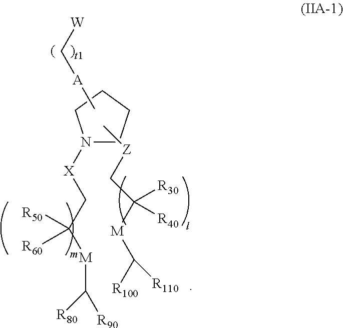

All the variables in this formula have been defined and exemplified as those described in the above embodiments.

In some embodiments, the ionizable lipid is represented by formula:

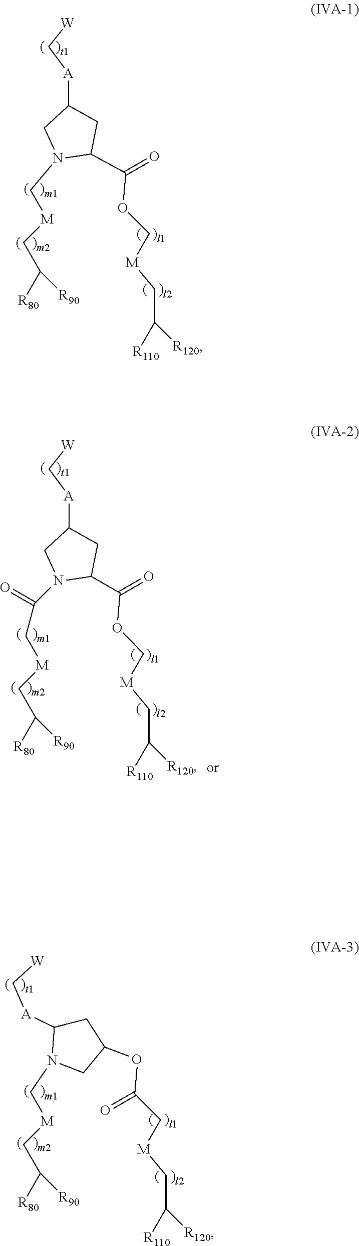

wherein:
A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each R⁷ is independently H, alkyl, alkenyl, cycloalkyl, hydroxy, hydroxyalkyl, or aminoalkyl;
t1 is an integer from 0 to 10;
W is hydroxyl, substituted or unsubstituted hydroxyalkyl, substituted or unsubstituted amino, substituted or unsubstituted aminocarbonyl, or substituted or unsubstituted heterocyclyl or heteroaryl;
each M is independently a biodegradable moiety;
each m1 is independently an integer from 3 to 6,
each l1 is independently an integer from 4 to 8,
m2 and 12 are each independently an integer from 0 to 3,
R₈₀ and R₉₀ are each independently unsubstituted C₅-C₈ alkyl; or R₈₀ is H or unsubstituted C₁-C₄ alkyl, and R₉₀ is unsubstituted C₅-C₁₁ alkyl; and
R₁₁₀ and R₁₂₀ are each independently unsubstituted C₅-C₈ alkyl; or R₁₁₀ is H or unsubstituted C₁-C₄ alkyl, and R₁₂₀ is unsubstituted C₅-C₁₁ alkyl. All the other variables in these formulas have been defined and exemplified as those described in the above embodiments. In some embodiments, in these formulas, R₈₀ is H or unsubstituted C₁-C₂ alkyl, and R₉₀ is unsubstituted C₆-C₁₀ alkyl; and R₁₁₀ and R₁₂₀ are each independently unsubstituted C₅-C₈ alkyl. In some embodiments, R₈₀, R₉₀, R₁₁₀, and R₁₂₀ are each independently unsubstituted C₅-C₈ alkyl.

In some embodiments, in the above formulas, A is absent, —O—, —N(R⁷)—, N(R⁷)C(O)—,

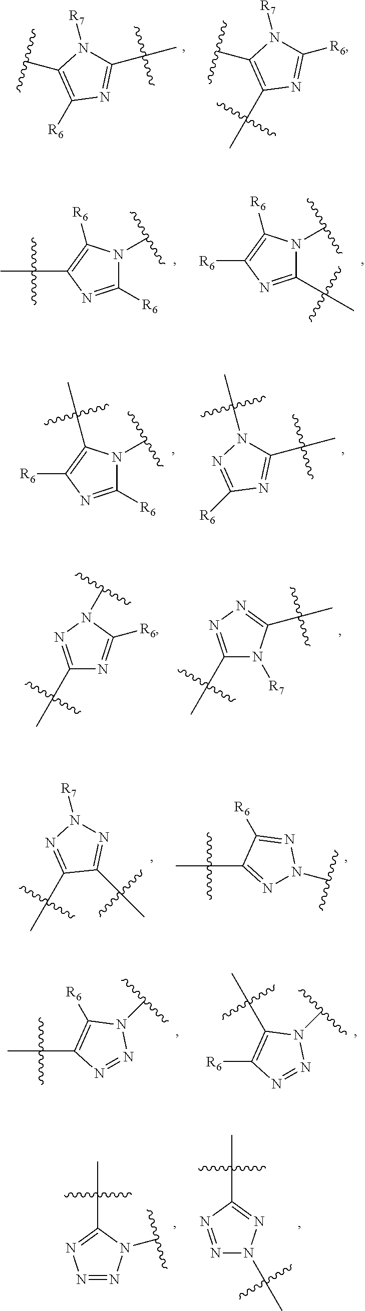

—OC(O)—, or —C(O)O—, wherein R⁶ is independently H, alkyl, hydroxyl, hydroxyalkyl, amino, aminoalkyl, thiol, thiolalkyl, or N⁺(R⁷)₃-alkylene-Q-; and R⁷ is H or C₁-C₃ alkyl.

In some embodiments, in the above formulas, t1 is 0, 1, 2, 3 or 4; and t is 0, 1, or 2.

In some embodiments, in the above formulas, W is hydroxyl, hydroxyalkyl, or one of the following:

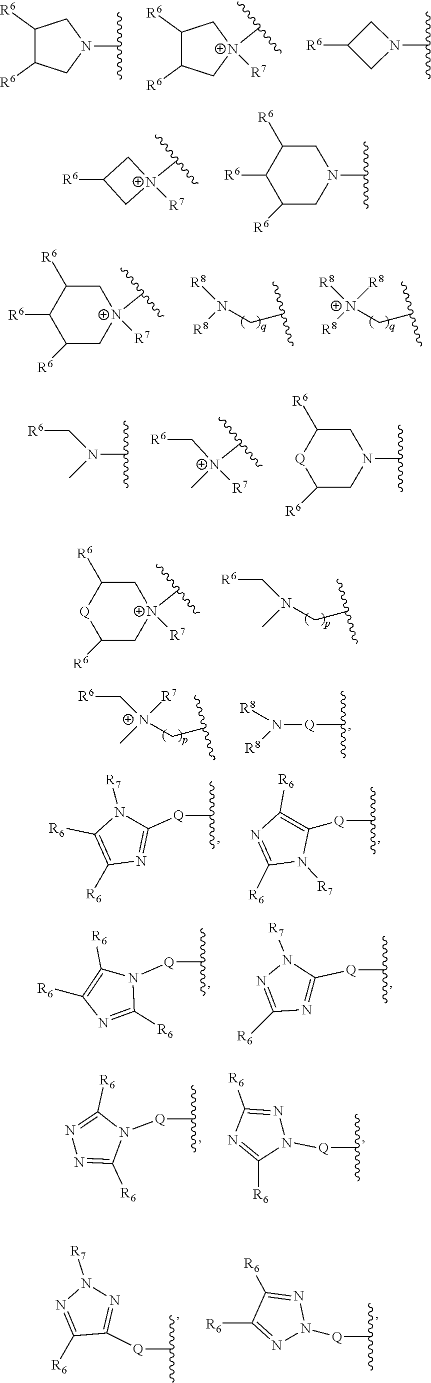

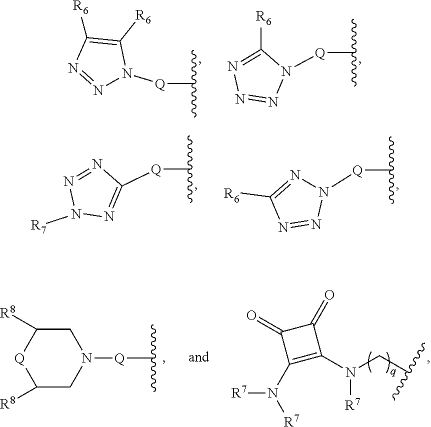

wherein:
each Q is independently absent, —O—, —C(O)—, —C(S)—, —C(O)O—, —C(R⁷)₂—, —C(O)N(R⁷)—, —C(S)N(R₇)—, or —N(R₇)—;
each R⁶ is independently H, alkyl, hydroxyl, hydroxyalkyl, alkoxy, amino, aminoalkyl, alkylamino, thiol, thiolalkyl, or N⁺(R⁷)₃-alkylene-Q-;
each R⁸ is independently H, alkyl, hydroxyalkyl, amino, aminoalkyl, thiol, or thiolalkyl, or two R⁸ together with the nitrogen atom may form a ring;
each q is independently 0, 1, 2, 3, 4, or 5; and
each p is independently 0, 1, 2, 3, 4, or 5.

In some embodiments, in the above formulas,
X is absent, —O—, or —C(O)—;
Z is —O—, —C(O)O—, or —OC(O)—;
M is —OC(O)— or —C(O)O—;
Y or

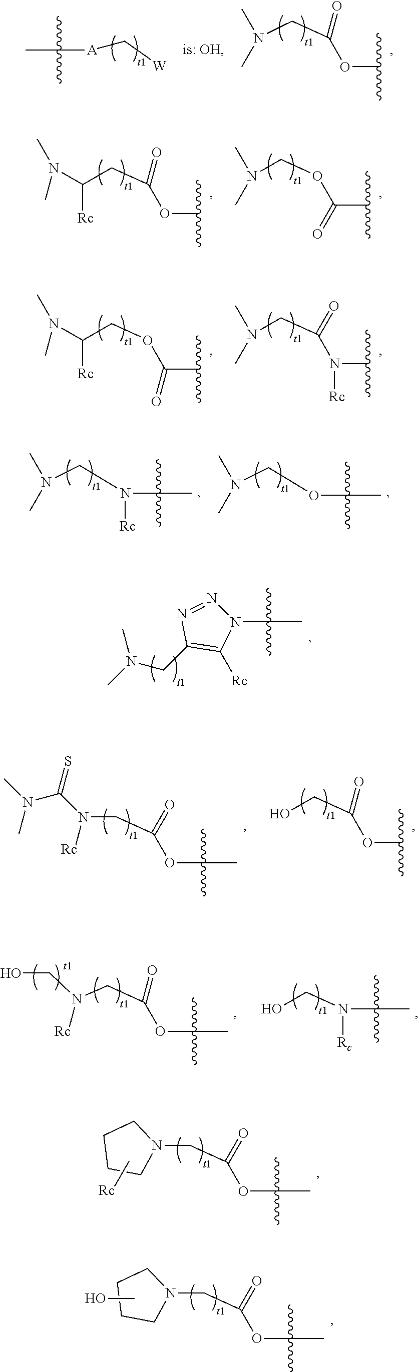

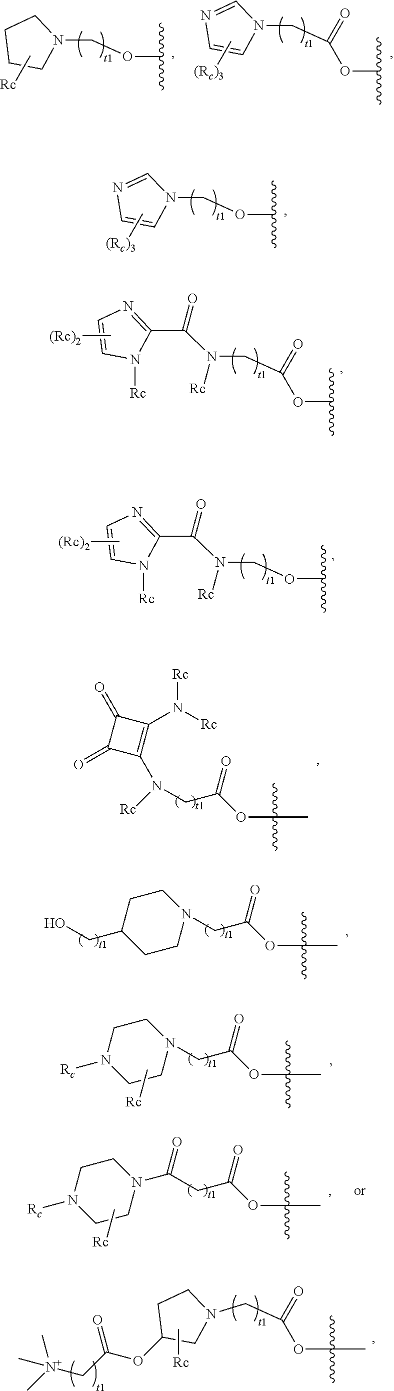

each R^{c} is independently H or C₁-C₃ alkyl;
each t1 is independently 1, 2, 3, or 4;
each of R₃₀, R₄₀, R₅₀, and R₆₀ is H or C₁-C₄ branched or unbranched alkyl;
R₇₀ is H; and each of R₈₀ and R₉₀ is independently H or C₁-C₁₂ branched or unbranched alkyl;
R₁₀₀ is H; and each of R₁₁₀ and R₁₂₀ is independently H or C₁-C₁₂ branched or unbranched alkyl, provided that at least one of R₈₀ and R₉₀ is not H, and at least one of R₁₀ and R₁₂₀ is not H;
l is from 3 to 7; and
m is from 1 to 5.

In some embodiments, in the above formulas, Y or

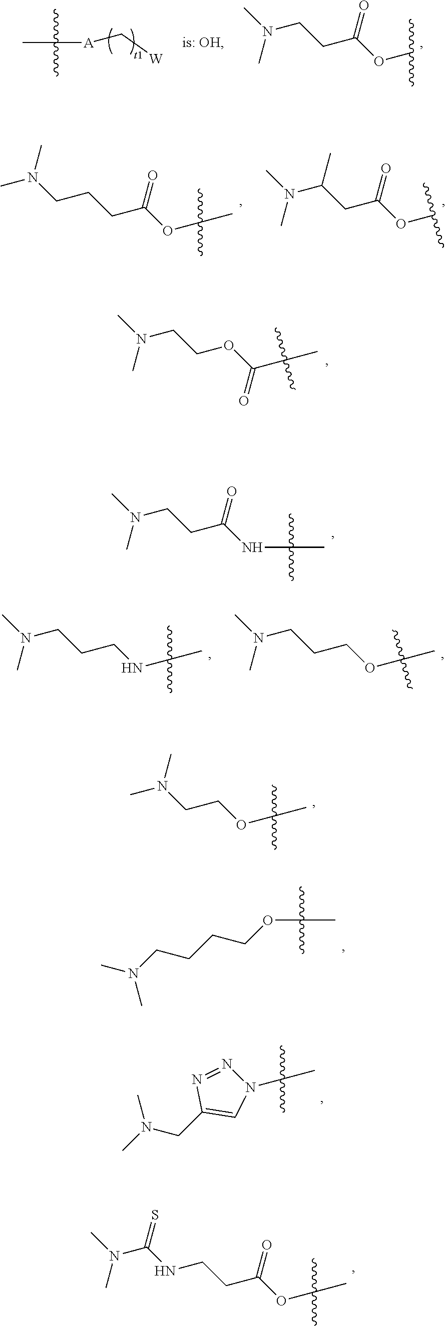

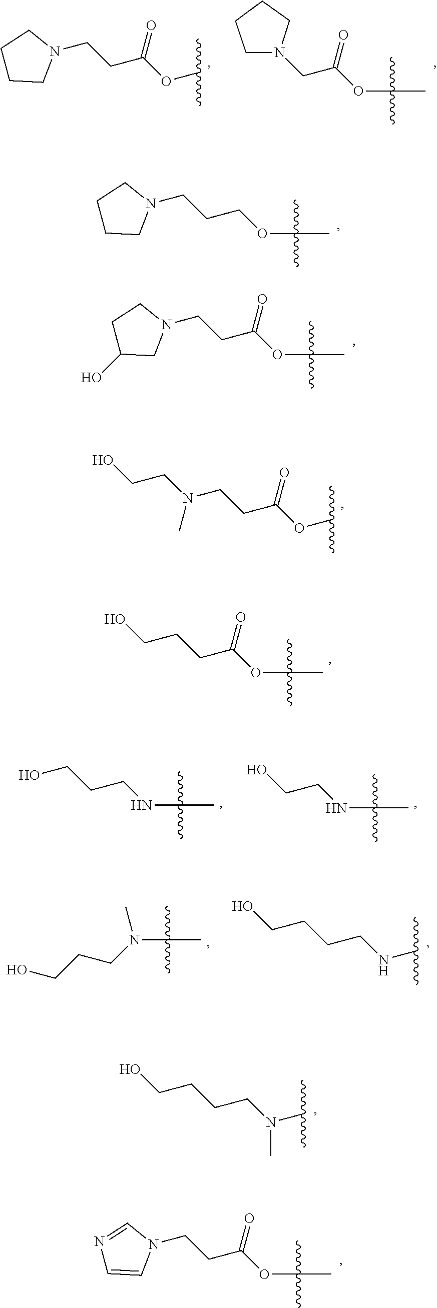

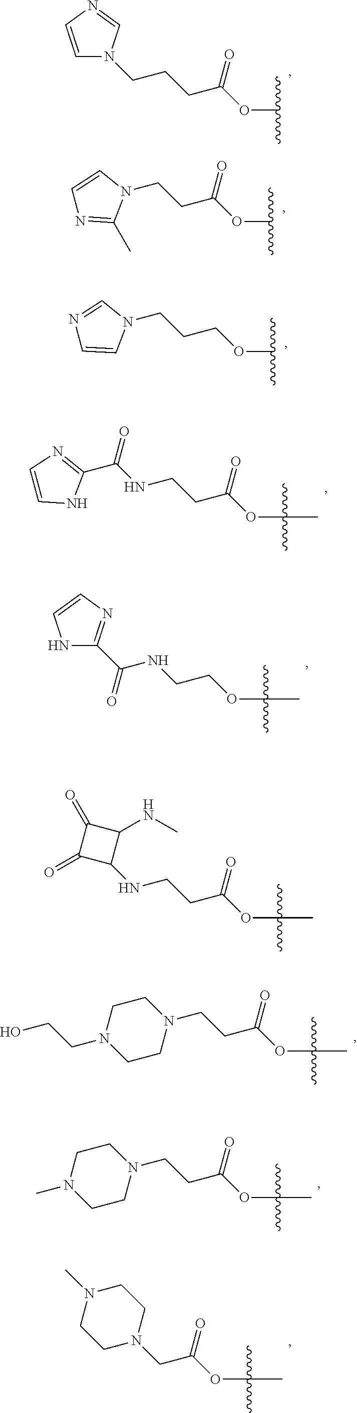

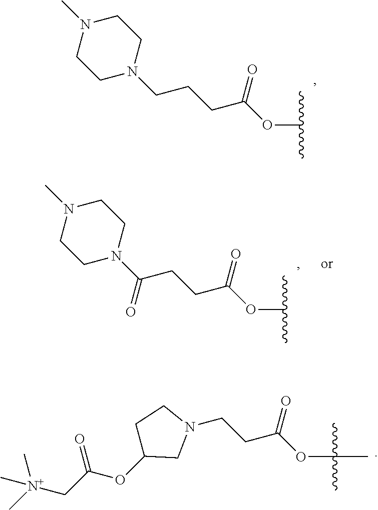 [SMILES: *OC(=O)CCCN1CCN(C)CC1 ; *OC(=O)CCC(=O)N1CCN(C)CC1 ; *OC(=O)CCN1CCC(OC(=O)C[N+](C)(C)C)C1]

More embodiments of the ionizable lipid of formula (II), in the Ionizable lipid compounds group ii), may be found in PCT Application No. PCT/US23/16300, filed on Mar. 24, 2023, the content of which is incorporated herein by reference in its entirety. In particular, all the ionizable lipids of formulas (I), (IA-1), (IA-2), (IIA)-(IIC), (IIA-1), (IIIA)-(IIIIE), (IIIC-1), (IVA-1)-(IVA-3), (IVC-1)-(IVC-2), (VA-1)-(VA-9), (VC-1)-(VC-6) of PCT Application No. PCT/US23/16300 are suitable for use as the ionizable lipids in this disclosure, and are incorporated herein by reference in its entirety.

Certain exemplary ionizable lipid compounds disclosed herein are set forth in Table II below.

TABLE II
Exemplary ionizable lipid compounds.

| Lipid | | |
|---|---|---|
| No. | Structure | Name |
| 2310 | 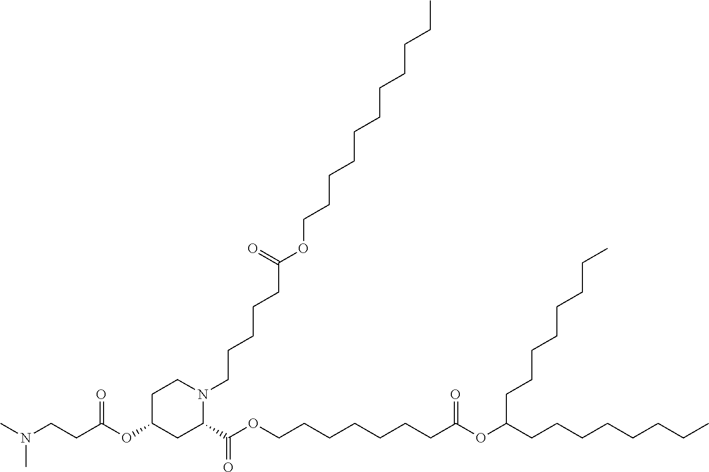 [SMILES: CCCCCCCCCCCOC(=O)CCCCCN1CC[C@@H](OC(=O)CCN(C)C)C[C@H]1C(=O)OCCCCCCCC(=O)OC(CCCCCCCC)CCCCCCCC] | heptadecan-9-yl 8-[(2S,4R)-4- {[3- (dimethylamino)propanoyl]oxy}- 1-[6-oxo-6- (undecyloxy)hexyl]piperidine- 2-carbonyloxy]octanoate |
| 2309 | 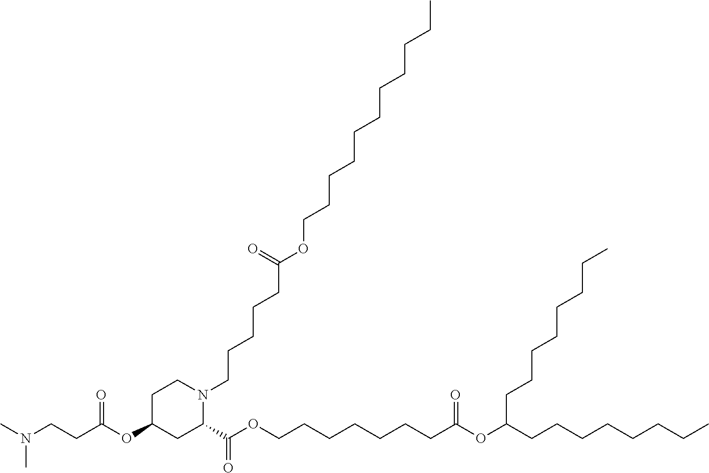 [SMILES: CCCCCCCCCCCOC(=O)CCCCCN1CC[C@H](OC(=O)CCN(C)C)C[C@H]1C(=O)OCCCCCCCC(=O)OC(CCCCCCCC)CCCCCCCC] | heptadecan-9-yl 8-[(2S,4S)-4- {[3- (dimethylamino)propanoyl]oxy}- 1-[6-oxo-6- (undecyloxy)hexyl]piperidine- 2-carbonyloxy]octanoate |
| 2308 | 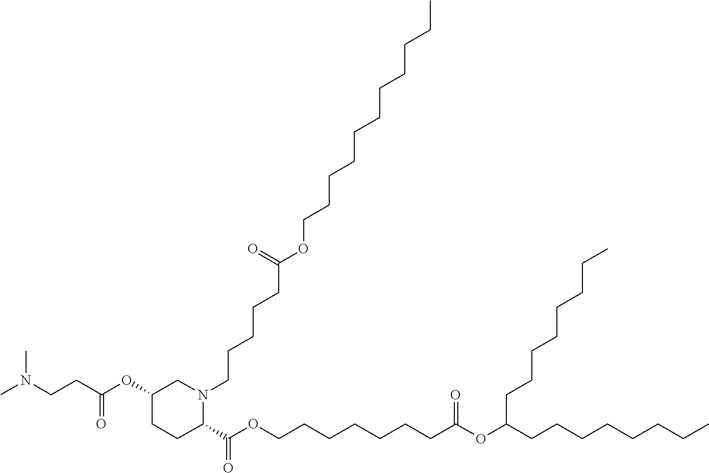 [SMILES: CCCCCCCCCCCOC(=O)CCCCCN1C[C@@H](OC(=O)CCN(C)C)CC[C@H]1C(=O)OCCCCCCCC(=O)OC(CCCCCCCC)CCCCCCCC] | heptadecan-9-yl 8-[(2S,5S)-5- {[3- (dimethylamino)propanoyl]oxy}- 1-[6-oxo-6- (undecyloxy)hexyl]piperidine- 2-carbonyloxy]octanoate |
| 2307 | 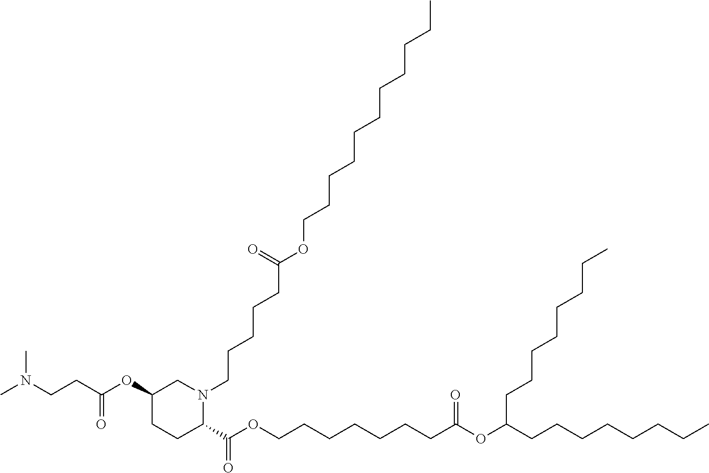 [SMILES: CCCCCCCCCCCOC(=O)CCCCCN1C[C@H](OC(=O)CCN(C)C)CC[C@H]1C(=O)OCCCCCCCC(=O)OC(CCCCCCCC)CCCCCCCC] | heptadecan-9-yl 8-[(2S,5R)-5- {[3- (dimethylamino)propanoyl]oxy}- 1-[6-oxo-6- (undecyloxy)hexyl]piperidine- 2-carbonyloxy]octanoate |
| 2306 | 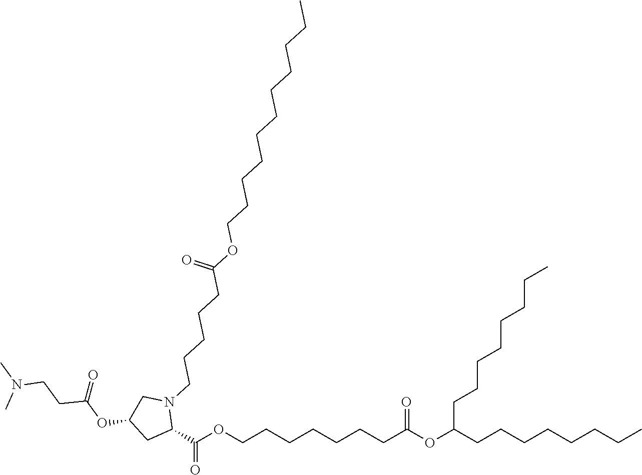 [SMILES: CCCCCCCCCCCOC(=O)CCCCCN1C[C@@H](OC(=O)CCN(C)C)C[C@H]1C(=O)OCCCCCCCC(=O)OC(CCCCCCCC)CCCCCCCC] | heptadecan-9-yl 8-[(2S,4S)-4- {[3- (dimethylamino)propanoyl]oxy}- 1-[6-oxo-6- (undecyloxy)hexyl]pyrrolidine- 2-carbonyloxy]octanoate |
| 2305 | 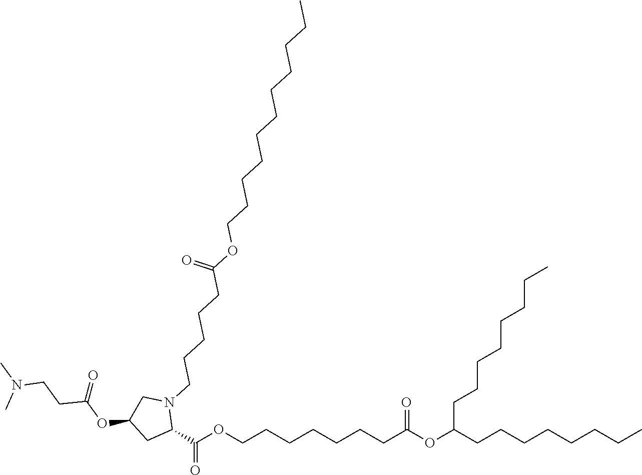 [SMILES: CCCCCCCCCCCOC(=O)CCCCCN1C[C@H](OC(=O)CCN(C)C)C[C@H]1C(=O)OCCCCCCCC(=O)OC(CCCCCCCC)CCCCCCCC] | heptadecan-9-yl 8-[(2S,4R)-4- {[3- (dimethylamino)propanoyl]oxy}- 1-[6-oxo-6- (undecyloxy)hexyl]pyrrolidine- 2-carbonyloxy]octanoate |
| 2304 | 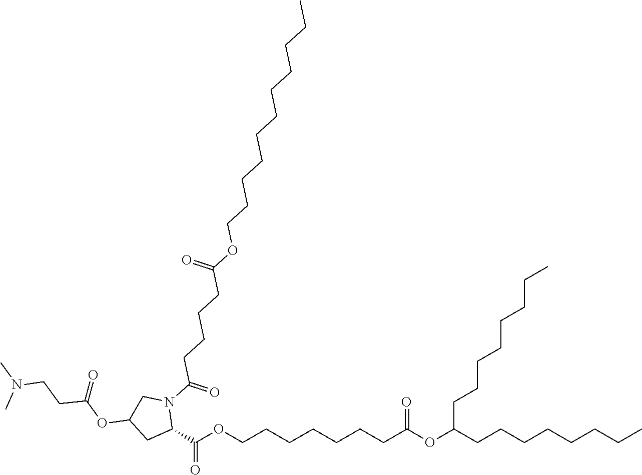 [SMILES: CCCCCCCCCCCOC(=O)CCCCC(=O)N1CC(OC(=O)CCN(C)C)C[C@H]1C(=O)OCCCCCCCC(=O)OC(CCCCCCCC)CCCCCCCC] | heptadecan-9-yl 8-[(2S)-4-{[3- (dimethylamino)propanoyl]oxy}- 1-[6-oxo-6- (undecyloxy)hexanoyl]pyrrolidine- 2-carbonyloxy]octanoate |
| 2298 | 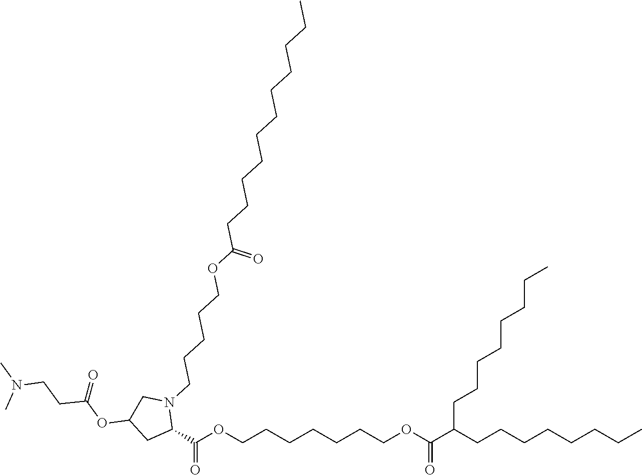 [SMILES: CCCCCCCCCCCC(=O)OCCCCCN1CC(OC(=O)CCN(C)C)C[C@H]1C(=O)OCCCCCCCOC(=O)C(CCCCCCCC)CCCCCCCC] | 5-[(2S)-4-{[3- (dimethylamino)propanoyl]oxy}- 2-[{{7-[(2- octyldecanoyl)oxy]heptyl}oxy) carbonyl]pyrrolidin-1-yl]pentyl dodecanoate |
| 2297 | 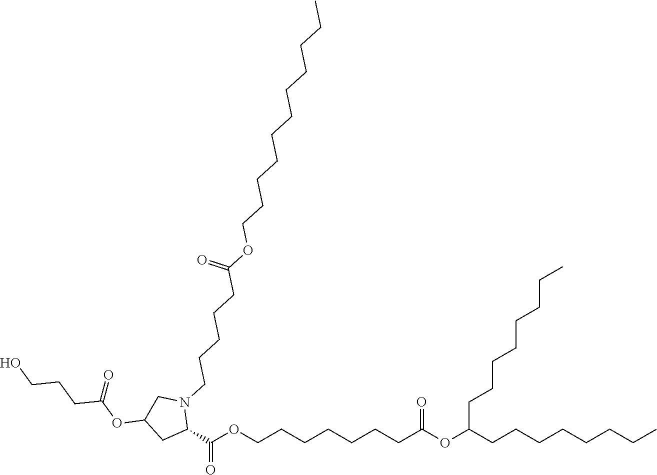 [SMILES: CCCCCCCCCCCOC(=O)CCCCCN1CC(OC(=O)CCCO)C[C@H]1C(=O)OCCCCCCCC(=O)OC(CCCCCCCC)CCCCCCCC] | heptadecan-9-yl 8-[(2S)-4-[(4- hydroxybutanoyl)oxy]-1-[6- oxo-6- (undecyloxy)hexyl]pyrrolidine- 2-carbonyloxy]octanoate |
| 2296 | 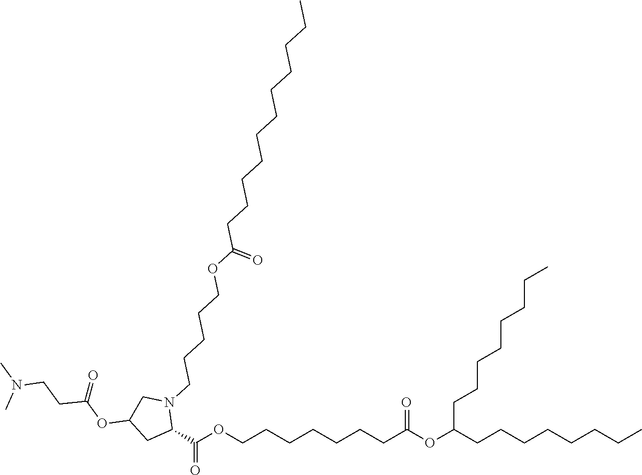 [SMILES: CCCCCCCCCCCC(=O)OCCCCCN1CC(OC(=O)CCN(C)C)C[C@H]1C(=O)OCCCCCCCC(=O)OC(CCCCCCCC)CCCCCCCC] | 5-[(2S)-4-{[3- (dimethylamino)propanoyl]oxy}- 2-({[8-(heptadecan-9- yloxy)-8- oxooctyl]oxy}carbonyl)pyrrolidin- 1-yl]pentyl dodecanoate |
| 2295 | 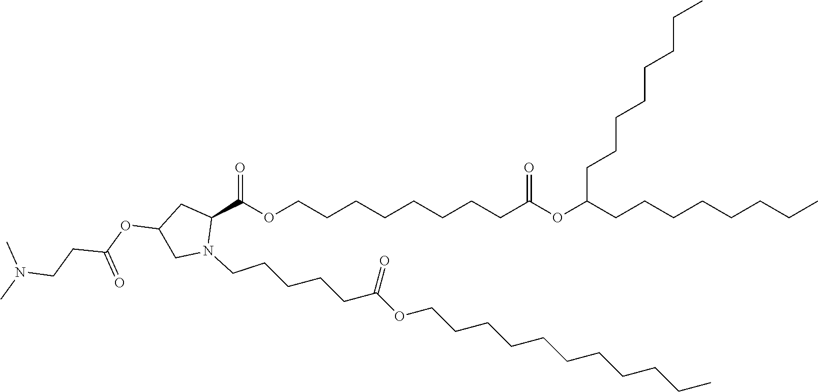 [SMILES: CCCCCCCCCCCOC(=O)CCCCCN1CC(OC(=O)CCN(C)C)C[C@H]1C(=O)OCCCCCCCCC(=O)OC(CCCCCCCC)CCCCCCCC] | heptadecan-9-yl 9-[(2S)-4-{[3- (dimethylamino)propanoyl]oxy}- 1-[6-oxo-6- (undecyloxy)hexyl]pyrrolidine- 2-carbonyloxy]nonanoate |
| 2294 | 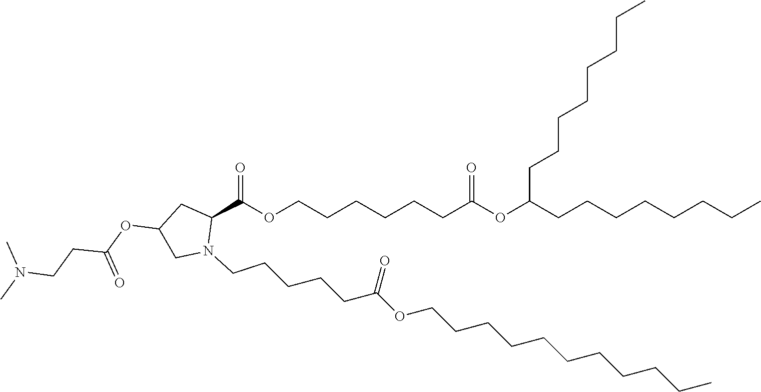 [SMILES: CCCCCCCCCCCOC(=O)CCCCCN1CC(OC(=O)CCN(C)C)C[C@H]1C(=O)OCCCCCCC(=O)OC(CCCCCCCC)CCCCCCCC] | heptadecan-9-yl 7-[(2S)-4-{[3- (dimethylamino)propanoyl]oxy}- 1-[6-oxo-6- (undecyloxy)hexyl]pyrrolidine- 2-carbonyloxy]heptanoate |
| 2293 | 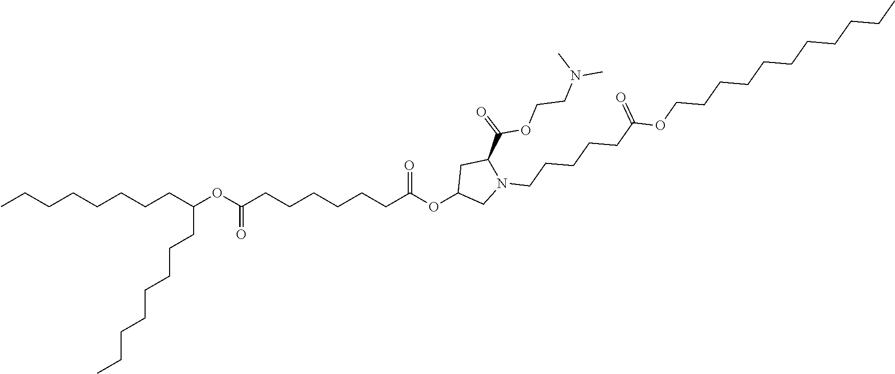 [SMILES: CCCCCCCCCCCOC(=O)CCCCCN1CC(OC(=O)CCCCCCC(=O)OC(CCCCCCCC)CCCCCCCC)C[C@H]1C(=O)OCCN(C)C] | (5S)-5-{[2- (dimethylamino)ethoxy]carbo- nyl}-1-[6-oxo-6- (undecyloxy)hexyl]pyrrolidin- 3-yl 1-heptadecan-9-yl octanedioate |
| 2292 | 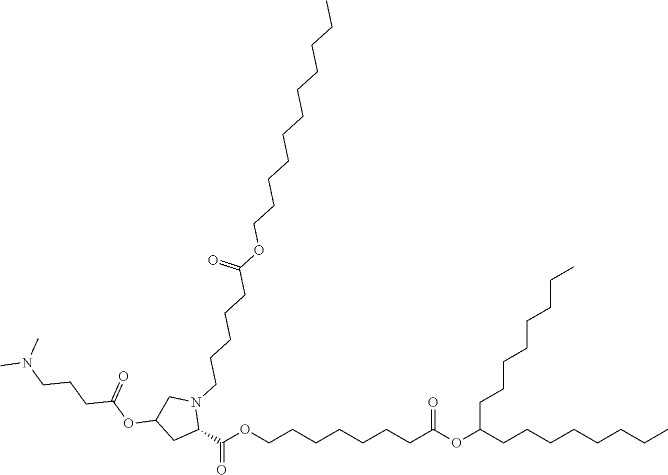 [SMILES: CCCCCCCCCCCOC(=O)CCCCCN1CC(OC(=O)CCCN(C)C)C[C@H]1C(=O)OCCCCCCCC(=O)OC(CCCCCCCC)CCCCCCCC] | heptadecan-9-yl 8-[(2S)-4-{[4- (dimethylamino)butanoyl]oxy}- 1-[6-oxo-6- (undecyloxy)hexyl]pyrrolidine- 2-carbonyloxy]octanoate |
| 2291 | 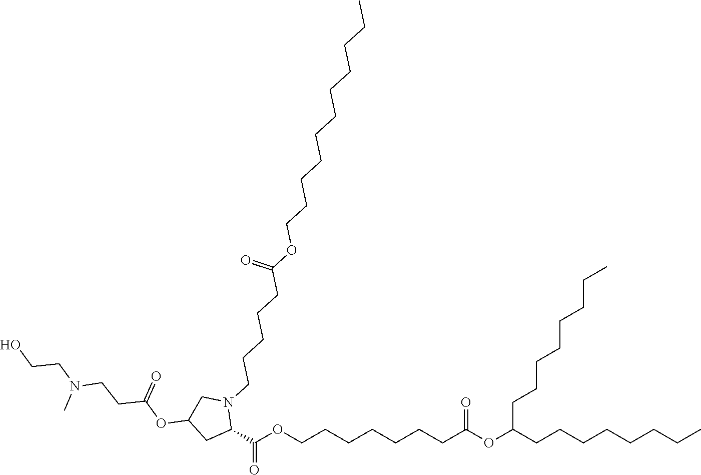 [SMILES: CCCCCCCCCCCOC(=O)CCCCCN1CC(OC(=O)CCN(C)CCO)C[C@H]1C(=O)OCCCCCCCC(=O)OC(CCCCCCCC)CCCCCCCC] | heptadecan-9-yl 8-[(2S)-4-({3- [(2- hydroxyethyl)(methyl)amino]pro- panoyl}oxy)-1-[6-oxo-6- (undecyloxy)hexyl]pyrrolidine- 2-carbonyloxy]octanoate |
| 2290 | 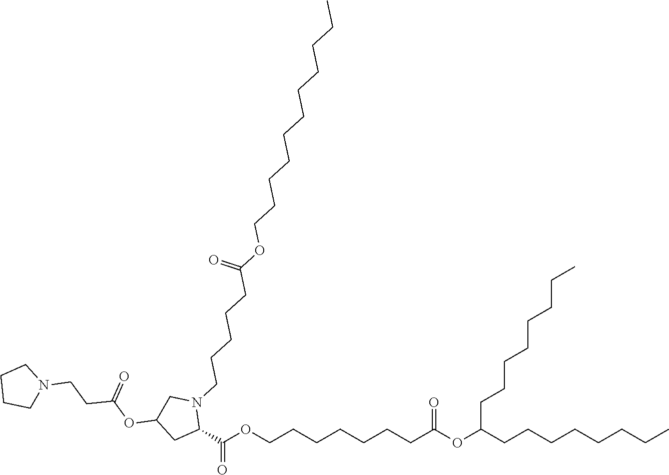 [SMILES: CCCCCCCCCCCOC(=O)CCCCCN1CC(OC(=O)CCN2CCCC2)C[C@H]1C(=O)OCCCCCCCC(=O)OC(CCCCCCCC)CCCCCCCC] | heptadecan-9-yl 8-[(2S)-1-[6- oxo-6-(undecyloxy)hexyl]-4- {[3-(pyrrolidin-1- yl)propanoyl]oxy}pyrrolidine- 2-carbonyloxy]oxtanoate |
| 2270 | 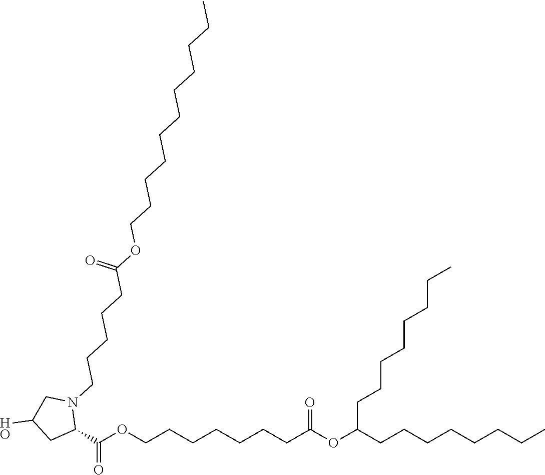 [SMILES: CCCCCCCCCCCOC(=O)CCCCCN1CC(O)C[C@H]1C(=O)OCCCCCCCC(=O)OC(CCCCCCCC)CCCCCCCC] | heptadecan-9-yl 8-[(2S)-4- hydroxy-1-[6-oxo-6- (undecyloxy)hexyl]pyrrolidine- 2-carbonyloxy]octanoate |
| 2260 | 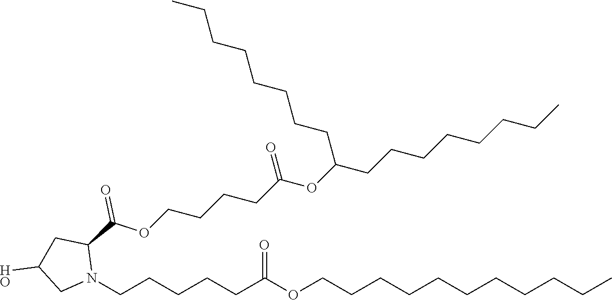 [SMILES: CCCCCCCCCCCOC(=O)CCCCCN1CC(O)C[C@H]1C(=O)OCCCCC(=O)OC(CCCCCCCC)CCCCCCCC] | undecyl 6-[(2S)-2-({[5- (heptadecan-9-yloxy)-5- oxopentyl]oxy}carbonyl)-4- hydroxypyrrolidin-1- yl]hexanoate |
| 2240 | 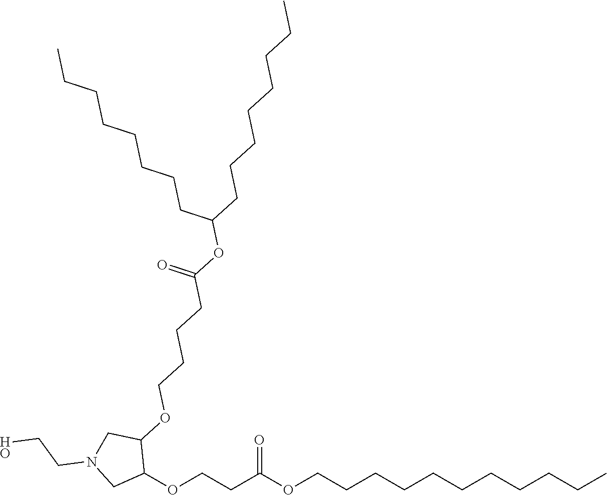 [SMILES: CCCCCCCCCCCOC(=O)CCOC1CN(CCO)CC1OCCCCC(=O)OC(CCCCCCCC)CCCCCCCC] | heptadecan-9-yl 5-{[1-(2- hydroxyethyl)-4-[3-oxo-3- (undecyloxy)propoxy]pyrrolidin- 3-yl]oxy}pentanoate |
| 2231 | 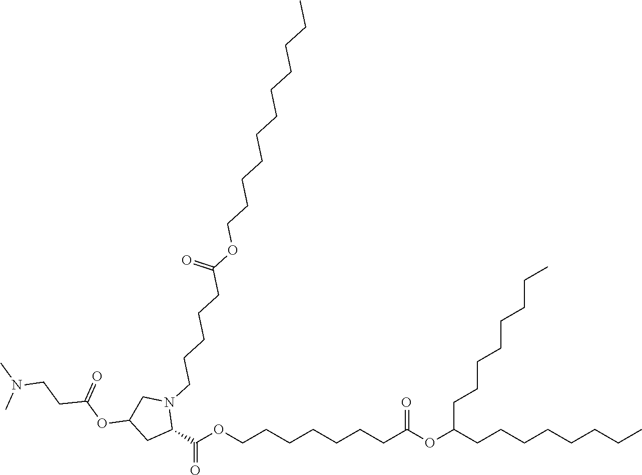 [SMILES: CCCCCCCCCCCOC(=O)CCCCCN1CC(OC(=O)CCN(C)C)C[C@H]1C(=O)OCCCCCCCC(=O)OC(CCCCCCCC)CCCCCCCC] | heptadecan-9-yl 8-[(2S)-4-{[3- (dimethylamino)propanoyl]oxy}- 1-[6-oxo-6- (undecyloxy)hexyl]pyrrolidine- 2-carbonyloxy]octanoate |
| 2230 | 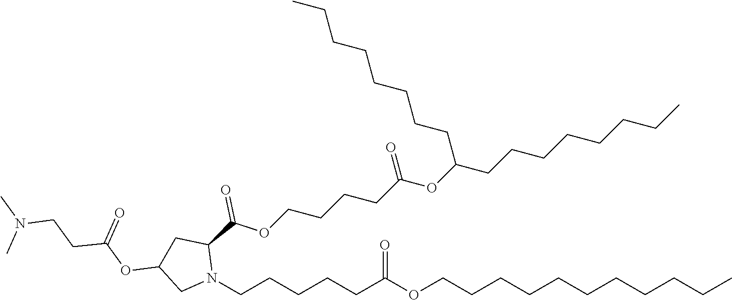 [SMILES: CCCCCCCCCCCOC(=O)CCCCCN1CC(OC(=O)CCN(C)C)C[C@H]1C(=O)OCCCCC(=O)OC(CCCCCCCC)CCCCCCCC] | undecyl 6-[(2S)-4-{[3- (dimethylamino)propanoyl]oxy}- 2-({[5-(heptadecan-9- yloxy)-5- oxopentyl]oxy}carbonyl)pyrroli- din-1-yl]hexanoate |
| 2329 | 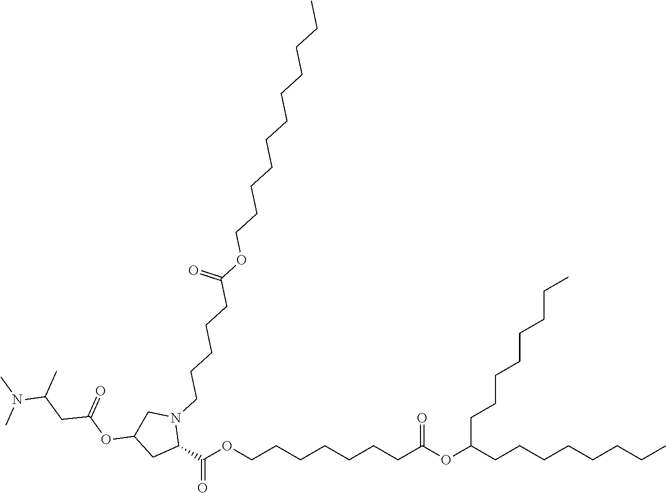 [SMILES: CCCCCCCCCCCOC(=O)CCCCCN1CC(OC(=O)CC(C)N(C)C)C[C@H]1C(=O)OCCCCCCCC(=O)OC(CCCCCCCC)CCCCCCCC] | 8-(heptadecan-9-yloxy)-8- oxooctyl (2S)-4-((3- (dimethylamino)butanoyl)oxy)- 1-(6-oxo-6- (undecyloxy)hexyl)pyrrolidine- 2-carboxylate |
| 2336 | 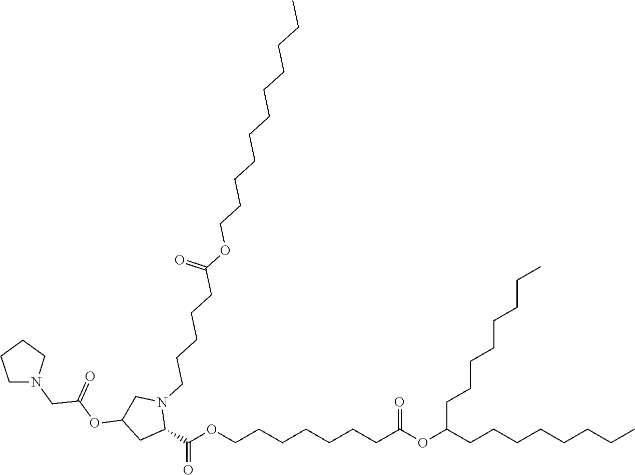 [SMILES: CCCCCCCCCCCOC(=O)CCCCCN1CC(OC(=O)CN2CCCC2)C[C@H]1C(=O)OCCCCCCCC(=O)OC(CCCCCCCC)CCCCCCCC] | 8-(heptadecan-9-yloxy)-8- oxooctyl (2S)-1-(6-oxo-6- (undecyloxy)hexyl)-4-(2- (pyrrolidin-1- yl)acetoxy)pyrrolidine-2- carboxylate |
| 2337 | 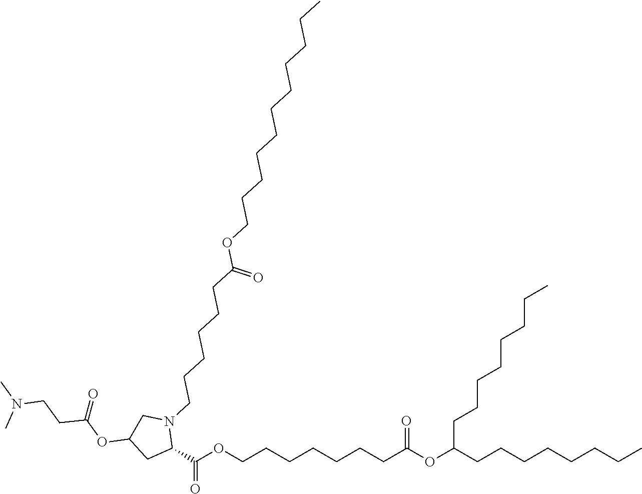 [SMILES: CCCCCCCCCCCOC(=O)CCCCCCN1CC(OC(=O)CCN(C)C)C[C@H]1C(=O)OCCCCCCCC(=O)OC(CCCCCCCC)CCCCCCCC] | 8-(heptadecan-9-yloxy)-8- oxooctyl (2S)-4-((3- (dimethylamino)propanoyl)oxy)- 1-(7-oxo-7- (undecyloxy)heptyl)pyrrolidine- 2-carboxylate |
| 2338 | 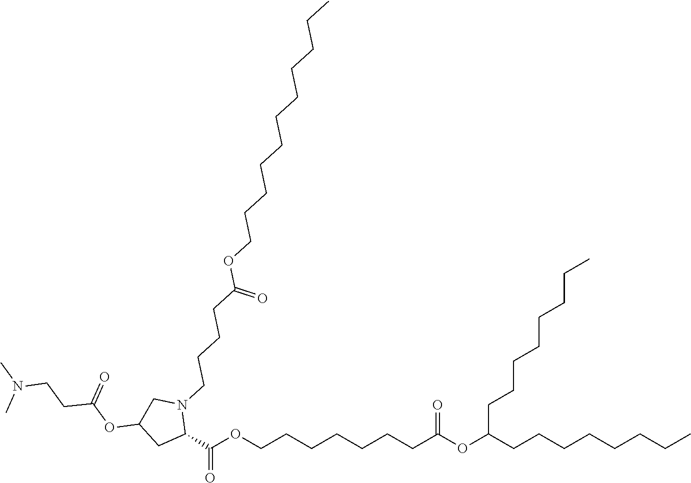 [SMILES: CCCCCCCCCCCOC(=O)CCCCN1CC(OC(=O)CCN(C)C)C[C@H]1C(=O)OCCCCCCCC(=O)OC(CCCCCCCC)CCCCCCCC] | 8-(heptadecan-9-yloxy)-8- oxooctyl (2S)-4-((3- (dimethylamino)propanoyl)oxy)- 1-(5-oxo-5- (undecyloxy)pentyl)pyrrolidine- 2-carboxylate |
| 2339 | 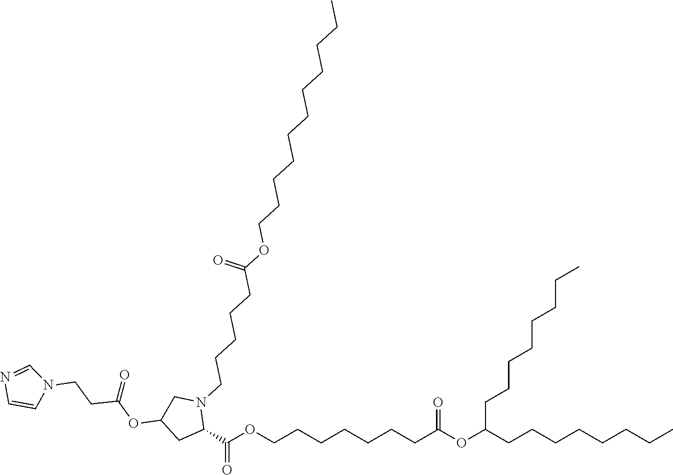 [SMILES: CCCCCCCCCCCOC(=O)CCCCCN1CC(OC(=O)CCn2ccnc2)C[C@H]1C(=O)OCCCCCCCC(=O)OC(CCCCCCCC)CCCCCCCC] | 8-(heptadecan-9-yloxy)-8- oxooctyl (2S)-4-((3-(1H- imidazol-1-yl)propanoyl)oxy)- 1-(6-oxo-6- (undecyloxy)hexyl)pyrrolidine- 2-carboxylate |
| 2340 | 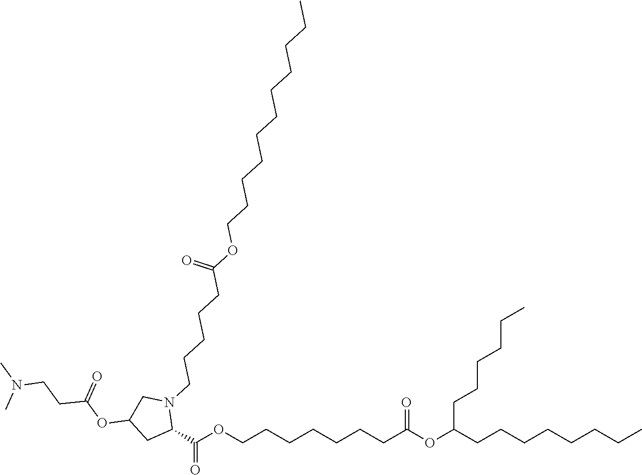 [SMILES: CCCCCCCCCCCOC(=O)CCCCCN1CC(OC(=O)CCN(C)C)C[C@H]1C(=O)OCCCCCCCC(=O)OC(CCCCCC)CCCCCCCC] | 8-oxo-8-(pentadecan-7- yloxy)octyl (2S)-4-((3- (dimethylamino)propanoyl)oxy)- 1-(6-oxo-6- (undecyloxy)hexyl)pyrrolidine- 2-carboxylate |
| 2341 | 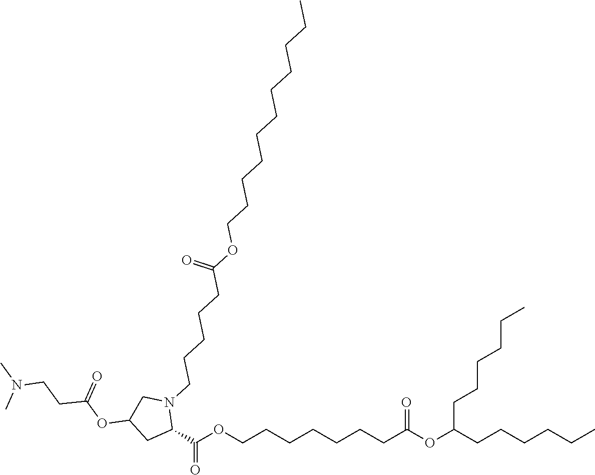 [SMILES: CCCCCCCCCCCOC(=O)CCCCCN1CC(OC(=O)CCN(C)C)C[C@H]1C(=O)OCCCCCCCC(=O)OC(CCCCCC)CCCCCC] | 8-oxo-8-(tridecan-7- yloxy)octyl (2S)-4-((3- (dimethylamino)propanoyl)oxy)- 1-(6-oxo-6- (undecayloxy)hexyl)pyrrolidine- 2-carboxylate |
| 2342 | 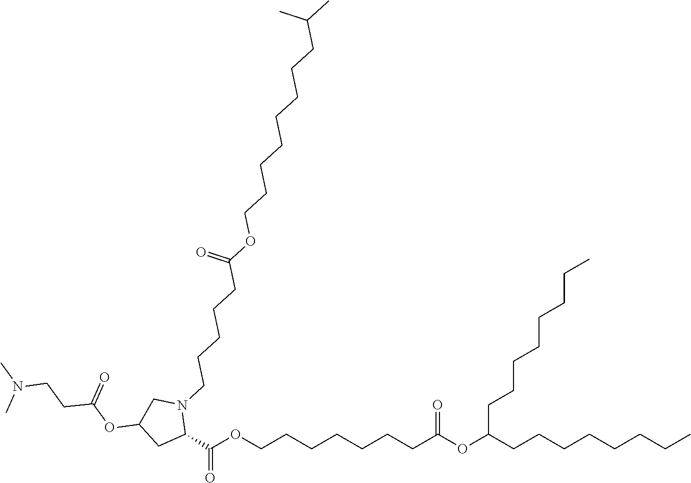 [SMILES: CCCCCCCCC(CCCCCCCC)OC(=O)CCCCCCCOC(=O)[C@@H]1CC(OC(=O)CCN(C)C)CN1CCCCCC(=O)OCCCCCCCCC(C)C] | 8-(heptadecan-9-yloxy)-8- oxooctyl (2S)-4-((3- (dimethylamino)propanoyl)oxy)- 1-(6-((9-methyldecyl)oxy)- 6-oxohexyl)pyrrolidine-2- carboxylate |
| 2343 | 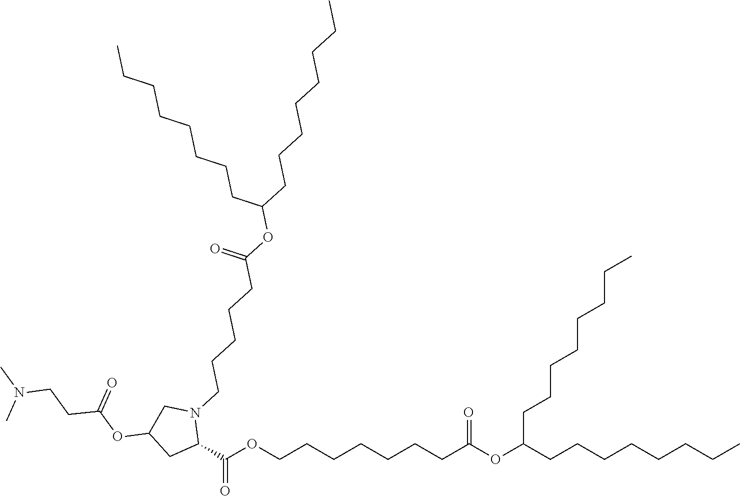 [SMILES: CCCCCCCCC(CCCCCCCC)OC(=O)CCCCCCCOC(=O)[C@@H]1CC(OC(=O)CCN(C)C)CN1CCCCCC(=O)OC(CCCCCCCC)CCCCCCCC] | 8-(heptadecan-9-yloxy)-8- oxooctyl (2S)-4-((3- (dimethylamino)propanoyl)oxy)- 1-(6-(heptadecan-9-yloxy)- 6-oxohexyl)pyrrolidine-2- carboxylate |
| 2344 | 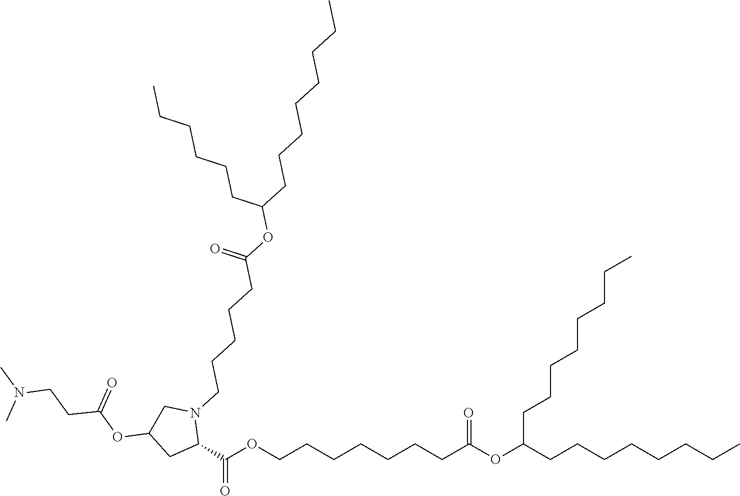 [SMILES: CCCCCCCCC(CCCCCC)OC(=O)CCCCCN1CC(OC(=O)CCN(C)C)C[C@H]1C(=O)OCCCCCCCC(=O)OC(CCCCCCCC)CCCCCCCC] | 8-(heptadecan-9-yloxy)-8- oxooctyl (2S)-4-((3- (dimethylamino)propanoyl)oxy)- 1-(6-oxo-6-(pentadecan-7- yloxy)hexyl)pyrrolidine-2- carboxylate |
| 2345 | 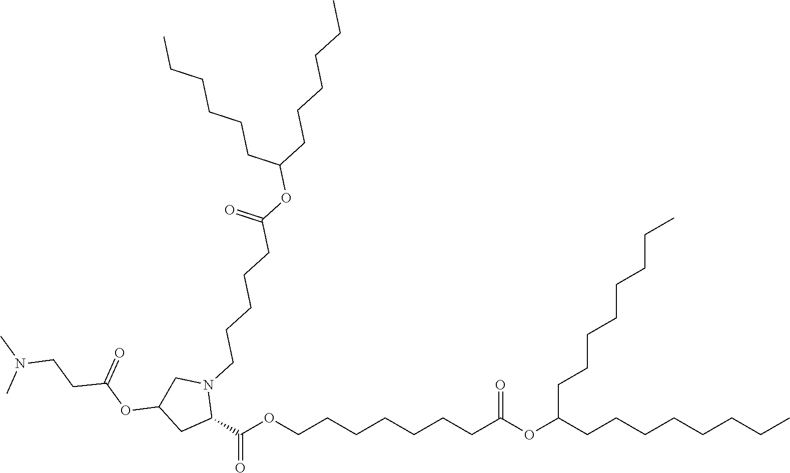 [SMILES: CCCCCCCCC(CCCCCCCC)OC(=O)CCCCCCCOC(=O)[C@@H]1CC(OC(=O)CCN(C)C)CN1CCCCCC(=O)OC(CCCCCC)CCCCCC] | 8-(heptadecan-9-yloxy)-8- oxooctyl (2S)-4-((3- (dimethylamino)propanoyl)oxy)- 1-(6-oxo-6-(tridecan-7- yloxy)hexyl)pyrrolidine-2- carboxylate |
| 2348 | 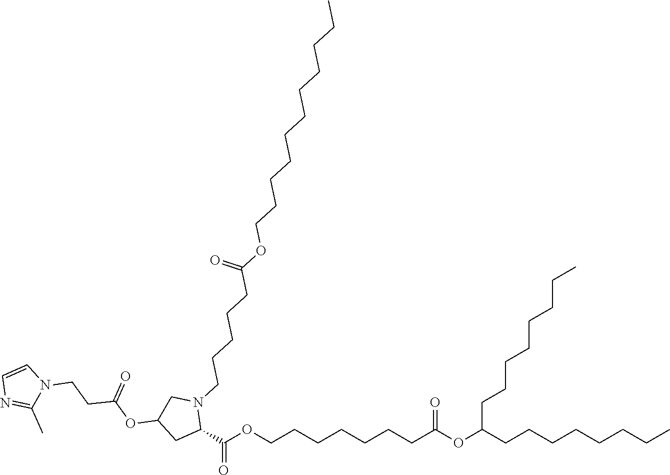 [SMILES: CCCCCCCCCCCOC(=O)CCCCCN1CC(OC(=O)CCn2ccnc2C)C[C@H]1C(=O)OCCCCCCCC(=O)OC(CCCCCCCC)CCCCCCCC] | 8-(heptadecan-9-yloxy)-8- oxooctyl (2S)-4-((3-(2-methyl- 1H-imidazol-1- yl)propanoyl)oxy)-1-(6-oxo-6- (undecyloxy)hexyl)pyrrolidine- 2-carboxylate |
| 2349 | 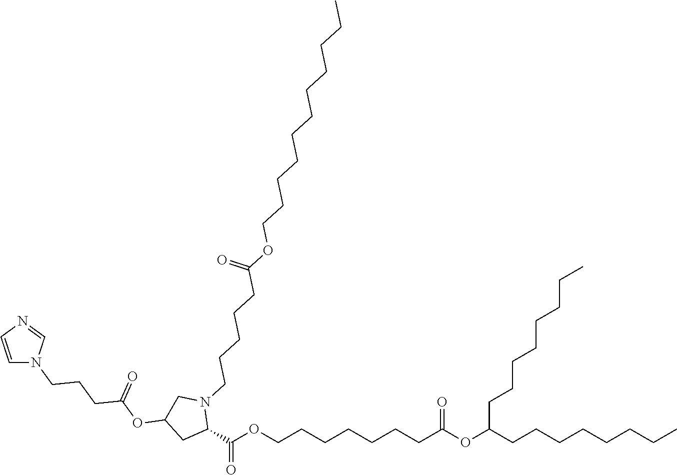 [SMILES: CCCCCCCCCCCOC(=O)CCCCCN1CC(OC(=O)CCCn2ccnc2)C[C@H]1C(=O)OCCCCCCCC(=O)OC(CCCCCCCC)CCCCCCCC] | 8-(heptadecan-9-yloxy)-8- oxooctyl (2S)-4-((4-(1H- imidazol-1-yl)butanoyl)oxy)-1- (6-oxo-6- (undecyloxy)hexyl)pyrrolidine- 2-carboxylate |
| 2352 | 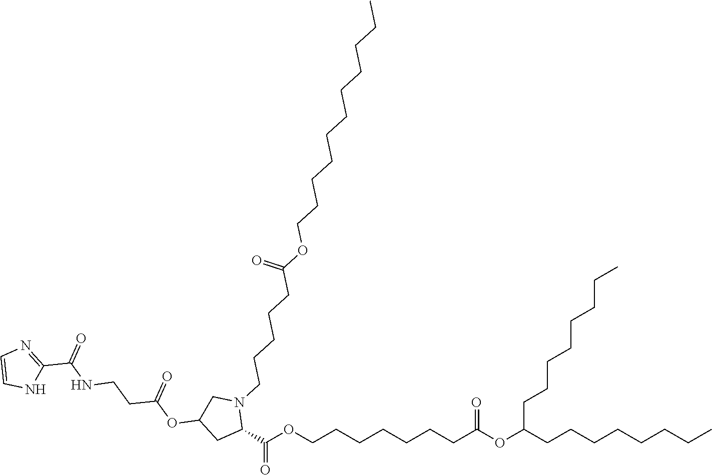 [SMILES: CCCCCCCCCCCOC(=O)CCCCCN1CC(OC(=O)CCNC(=O)c2ncc[nH]2)C[C@H]1C(=O)OCCCCCCCC(=O)OC(CCCCCCCC)CCCCCCCC] | 8-(heptadecan-9-yloxy)-8- oxooctyl (2S)-4-((3-(1H- imidazole-2- carboxamido)propanoyl)oxy)- 1-(6-oxo-6- (undecyloxy)hexyl)pyrrolidine- 2-carboxylate |
| 2371 | 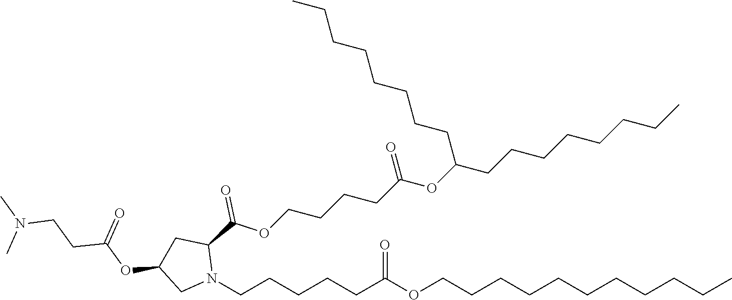 [SMILES: CCCCCCCCCCCOC(=O)CCCCCN1C[C@@H](OC(=O)CCN(C)C)C[C@H]1C(=O)OCCCCC(=O)OC(CCCCCCCC)CCCCCCCC] | 5-(heptadecan-9-yloxy)-5- oxopentyl (2S,4S)-4-((3- (dimethylamino)propanoyl)oxy)- 1-(6-oxo-6- (undecyloxy)hexyl)pyrrolidine- 2-carboxylate |
| 2372 | 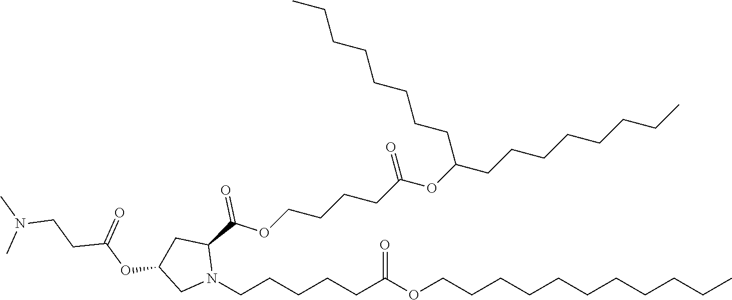 [SMILES: CCCCCCCCCCCOC(=O)CCCCCN1C[C@H](OC(=O)CCN(C)C)C[C@H]1C(=O)OCCCCC(=O)OC(CCCCCCCC)CCCCCCCC] | 5-(heptadecan-9-yloxy)-5- oxopentyl (2S,4R)-4-((3- (dimethylamino)propanoyl)oxy)- 1-(6-oxo-6- (undecyloxy)hexyl)pyrrolidine- 2-carboxylate |
| 2373 | 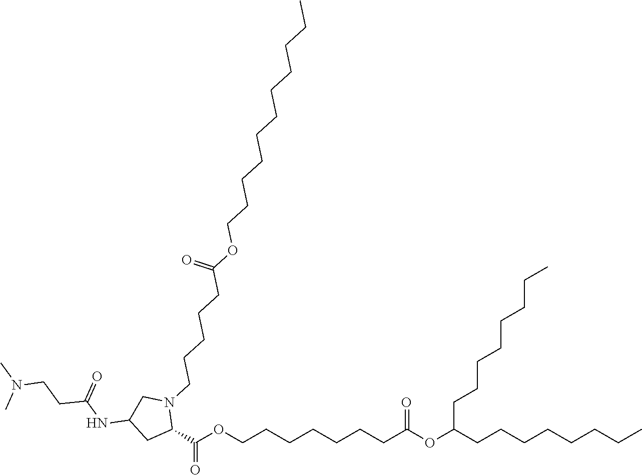 [SMILES: CCCCCCCCCCCOC(=O)CCCCCN1CC(NC(=O)CCN(C)C)C[C@H]1C(=O)OCCCCCCCC(=O)OC(CCCCCCCC)CCCCCCCC] | 8-(heptadecan-9-yloxy)-8- oxooctyl (2S)-4-(3- (dimethylamino)propanamido)- 1-(6-oxo-6- (undecyloxy)hexyl)pyrrolidine- 2-carboxylate |
| 2375 | 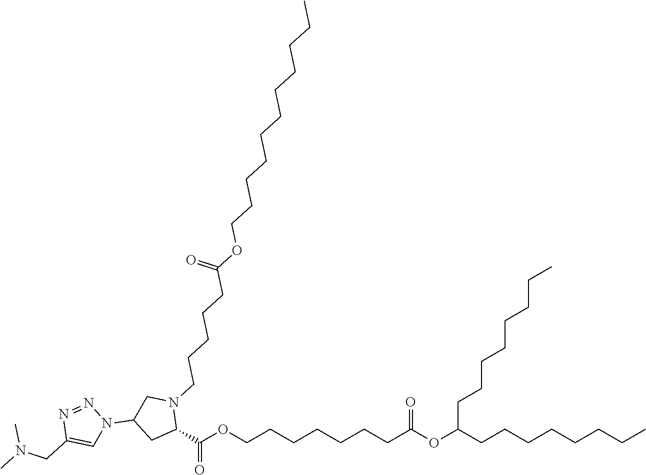 [SMILES: CCCCCCCCCCCOC(=O)CCCCCN1CC(n2cc(CN(C)C)nn2)C[C@H]1C(=O)OCCCCCCCC(=O)OC(CCCCCCCC)CCCCCCCC] | 8-(heptadecan-9-yloxy)-8- oxooctyl (2S)-4-(4- ((dimethylamino)methyl)-1H- 1,2,3-triazol-1-yl)-1-(6-oxo-6- (undecyloxy)hexyl)pyrrolidine- 2-carboxylate |
| 2376 | 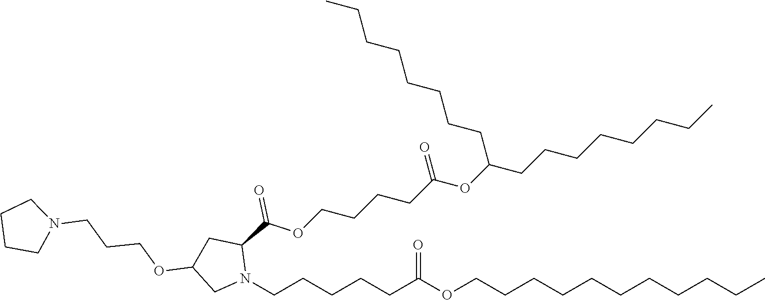 [SMILES: CCCCCCCCCCCOC(=O)CCCCCN1CC(OCCCN2CCCC2)C[C@H]1C(=O)OCCCCC(=O)OC(CCCCCCCC)CCCCCCCC] | 5-(heptadecan-9- yloxy)-5-oxopentyl (2S)-1-(6-oxo-6- (undecyloxy)hexyl)-4- (3-(pyrrolidin-1- yl)propoxy)pyrrolidine- 2-carboxylate |
| 2377 | 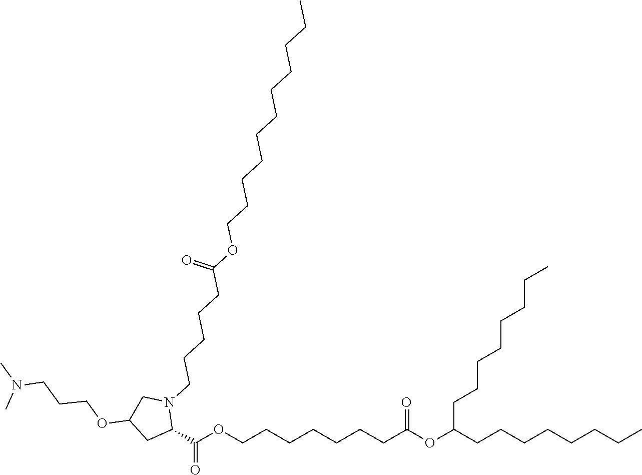 [SMILES: CCCCCCCCCCCOC(=O)CCCCCN1CC(OCCCN(C)C)C[C@H]1C(=O)OCCCCCCCC(=O)OC(CCCCCCCC)CCCCCCCC] | 8-(heptadecan-9-yloxy)-8- oxooctyl (2S)-4-(3- (dimethylamino)propoxy)-1- (6-oxo-6- (undecyloxy)hexyl)pyrrolidine- 2-carboxylate |
| 2436 | 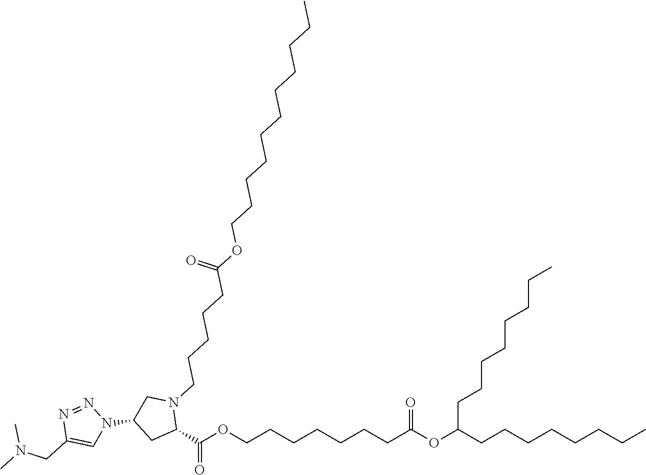 [SMILES: CCCCCCCCCCCOC(=O)CCCCCN1C[C@@H](n2cc(CN(C)C)nn2)C[C@H]1C(=O)OCCCCCCCC(=O)OC(CCCCCCCC)CCCCCCCC] | 8-(heptadecan-9-yloxy)-8- oxooctyl (2S,4S)-4-(4- ((dimethylamino)methyl)- 1H-1,2,3-triazol-1-yl)-1-(6- oxo-6- (undecyloxy)hexyl)pyrrolidine- 2-carboxylate |
| 2437 | 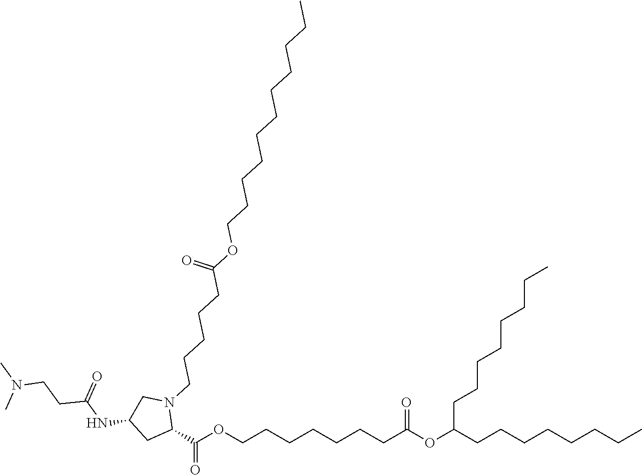 [SMILES: CCCCCCCCCCCOC(=O)CCCCCN1C[C@@H](NC(=O)CCN(C)C)C[C@H]1C(=O)OCCCCCCCC(=O)OC(CCCCCCCC)CCCCCCCC] | 8-(heptadecan-9-yloxy)-8- oxooctyl (2S,4S)-4-(3- (dimethylamino)propanamido)- 1-(6-oxo-6- (undecyloxy)hexyl)pyrrolidine- 2-carboxylate |
| 2440 | 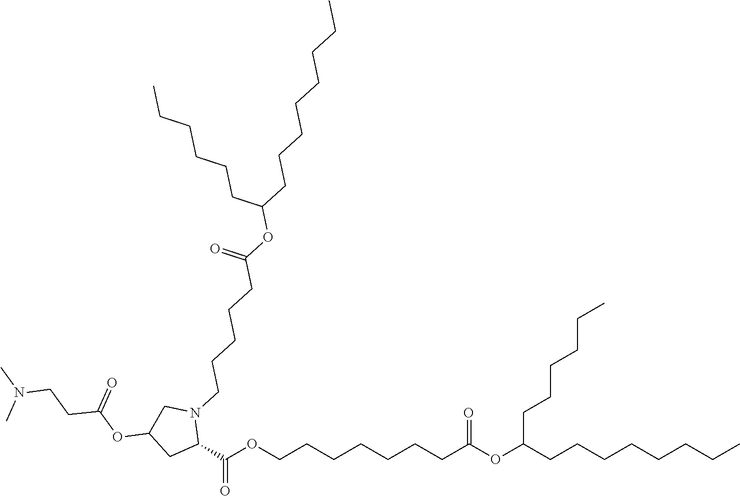 [SMILES: CCCCCCCCC(CCCCCC)OC(=O)CCCCCCCOC(=O)[C@@H]1CC(OC(=O)CCN(C)C)CN1CCCCCC(=O)OC(CCCCCC)CCCCCCCC] | 8-oxo-8-(pentadecan-7- yloxy)octyl (2S)-4-((3- (dimethylamino)propanoyl)oxy)- 1-(6-oxo-6-(pentadecan-7- yloxy)hexyl)pyrrolidine-2- carboxylate |
| 2452 | 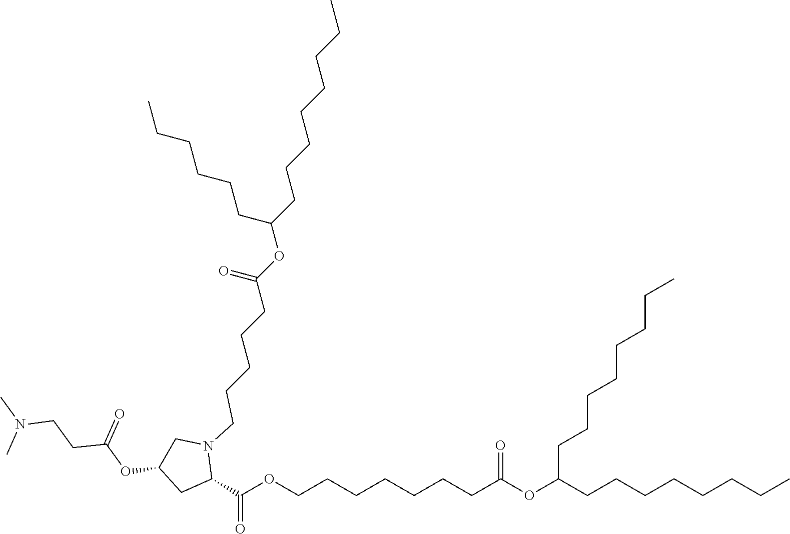 [SMILES: CCCCCCCCC(CCCCCC)OC(=O)CCCCCN1C[C@@H](OC(=O)CCN(C)C)C[C@H]1C(=O)OCCCCCCCC(=O)OC(CCCCCCCC)CCCCCCCC] | 8-(heptadecan-9-yloxy)-8- oxooctyl (2S,4S)-4-((3- (dimethylamino)propanoyl)oxy)- 1-(6-oxo-6-(pentadecan-7- yloxy)hexyl)pyrrolidine-2- carboxylate |
| 2232 | 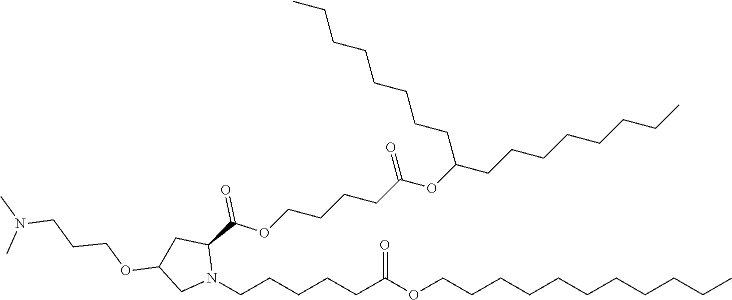 [SMILES: CCCCCCCCCCCOC(=O)CCCCCN1CC(OCCCN(C)C)C[C@H]1C(=O)OCCCCC(=O)OC(CCCCCCCC)CCCCCCCC] | undecyl 6-[(2S)-4-[3- (dimethylamino)propoxy]-2- ({[5-(heptadecan-9-yloxy)-5- oxopentyl]oxy}carbonyl)pyrroli- din-1-yl]hexanoate |
| 2350 | 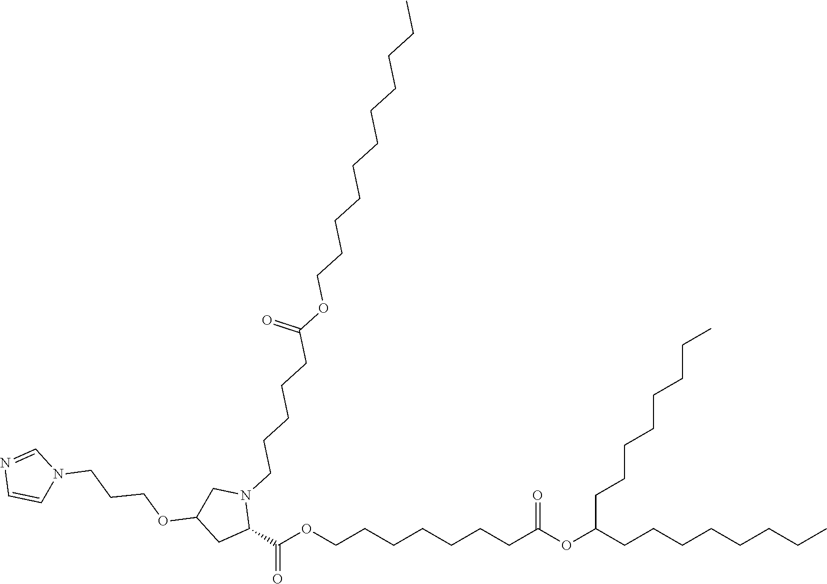 [SMILES: CCCCCCCCCCCOC(=O)CCCCCN1CC(OCCCn2ccnc2)C[C@H]1C(=O)OCCCCCCCC(=O)OC(CCCCCCCC)CCCCCCCC] | 8-(heptadecan-9-yloxy)-8- oxooctyl (2S)-4-(3-(1H- imidazol-1-yl)propoxy)-1-(6- oxo-6- (undecyloxy)hexyl)pyrrolidine- 2-carboxylate |
| 2351 | 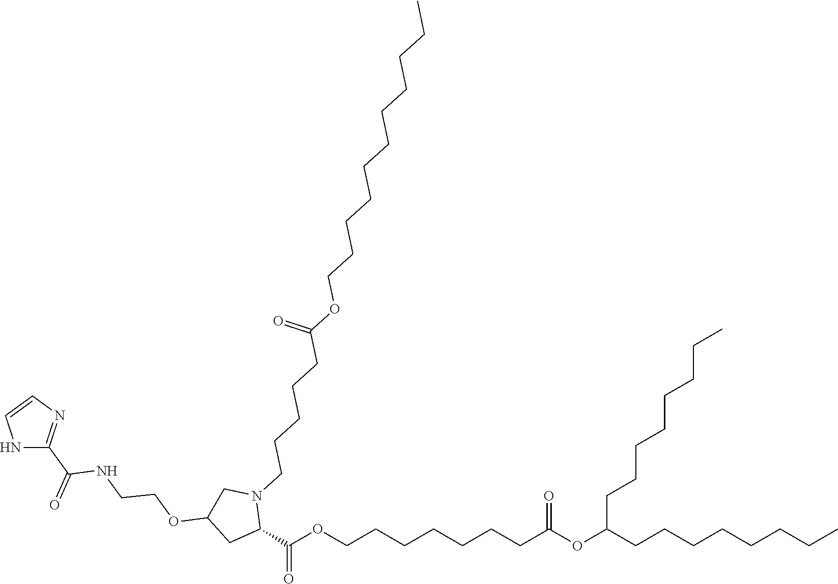 [SMILES: CCCCCCCCCCCOC(=O)CCCCCN1CC(OCCNC(=O)c2ncc[nH]2)C[C@H]1C(=O)OCCCCCCCC(=O)OC(CCCCCCCC)CCCCCCCC] | 8-(heptadecan-9-yloxy)-8- oxooctyl (2S)-4-(2-(1H- imidazole-2- carboxamido)ethoxy)-1-(6- oxo-6- (undecyloxy)hexyl)pyrrolidine- 2-carboxylate |
| 2359 | 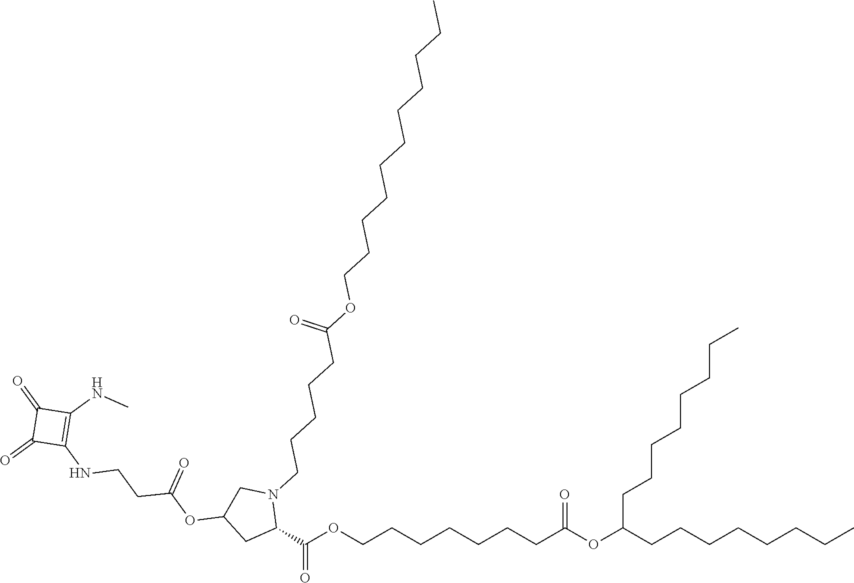 [SMILES: CCCCCCCCCCCOC(=O)CCCCCN1CC(OC(=O)CCNc2c(NC)c(=O)c2=O)C[C@H]1C(=O)OCCCCCCCC(=O)OC(CCCCCCCC)CCCCCCCC] | 8-(heptadecan-9-yloxy)-8- oxooctyl (2S)-4-((3-((2- (methylamino)-3,4- dioxocyclobut-1-en-1- yl)amino)propanoyl)oxy)-1-(6- oxo-6- (undecyloxy)hexyl)pyrrolidine- 2-carboxylate |
| 2360 | 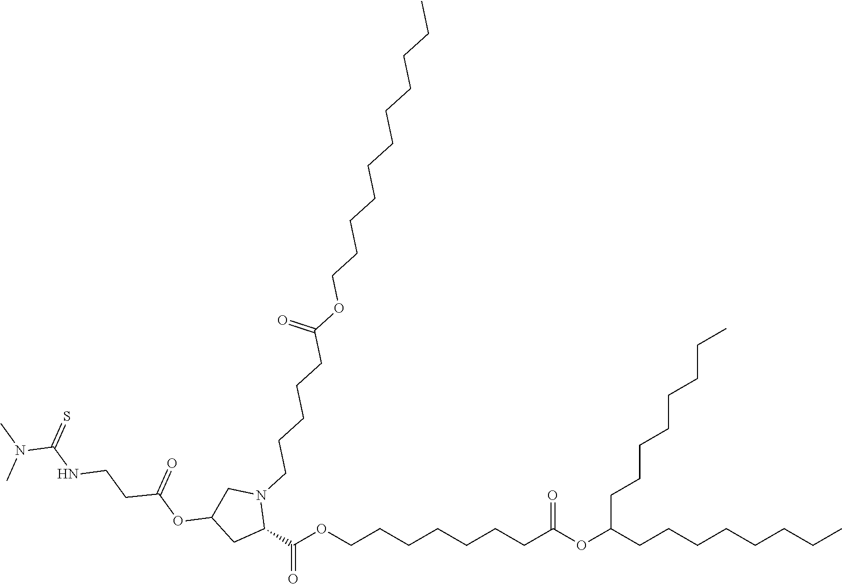 [SMILES: CCCCCCCCCCCOC(=O)CCCCCN1CC(OC(=O)CCNC(=S)N(C)C)C[C@H]1C(=O)OCCCCCCCC(=O)OC(CCCCCCCC)CCCCCCCC] | 8-(heptadecan-9-yloxy)-8- oxooctyl (2S)-4-((3-(3,3- dimethylthioureido)propanoyl) oxy)-1-(6-oxo-6- (undecyloxy)hexyl)pyrrolidine- 2-carboxylate |
| 2363 | 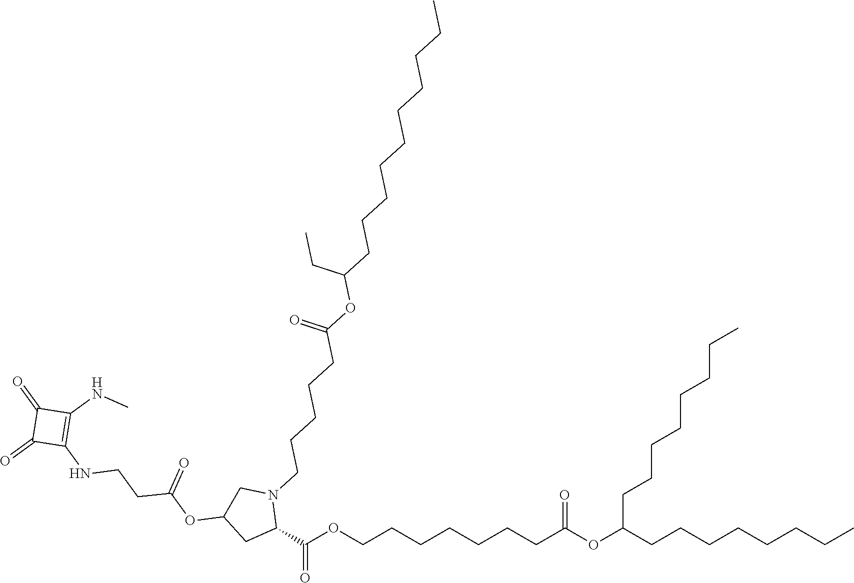 [SMILES: CCCCCCCCCCC(CC)OC(=O)CCCCCN1CC(OC(=O)CCNc2c(NC)c(=O)c2=O)C[C@H]1C(=O)OCCCCCCCC(=O)OC(CCCCCCCC)CCCCCCCC] | 8-(heptadecan-9-yloxy)-8- oxooctyl (2S)-4-((3-((2- (methylamino)-3,4- dioxocyclobut-1-en-1- yl)amino)propanoyl)oxy)-1-(6- oxo-6-(tridecan-3- yloxy)hexyl)pyrrolidine-2- carboxylate |
| 2364 | 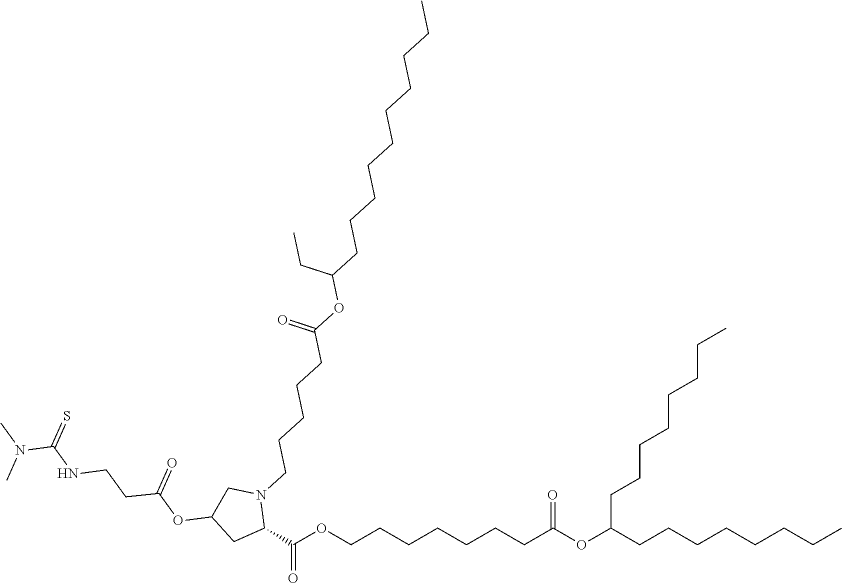 [SMILES: CCCCCCCCCCC(CC)OC(=O)CCCCCN1CC(OC(=O)CCNC(=S)N(C)C)C[C@H]1C(=O)OCCCCCCCC(=O)OC(CCCCCCCC)CCCCCCCC] | 8-(heptadecan-9-yloxy)-8- oxooctyl (2S)-4-((3-(3,3- dimethylthioureido)propanoyl) oxy)-1-(6-oxo-6-(tridecan-3- yloxy)hexyl)pyrrolidine-2- carboxylate |
| 2374 | 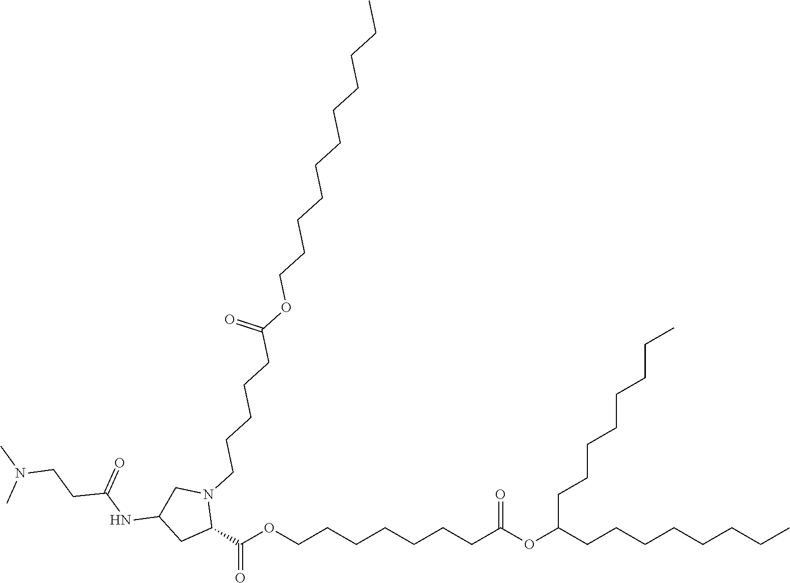 [SMILES: CCCCCCCCCCCOC(=O)CCCCCN1CC(NC(=O)CCN(C)C)C[C@H]1C(=O)OCCCCCCCC(=O)OC(CCCCCCCC)CCCCCCCC] | 8-(heptadecan-9-yloxy)-8- oxooctyl (2S)-4-((3- (dimethylamino)propyl)amino)- 1-(6-oxo-6- (undecyloxy)hexyl)pyrrolidine- 2-carboxylate |
| 2422 | 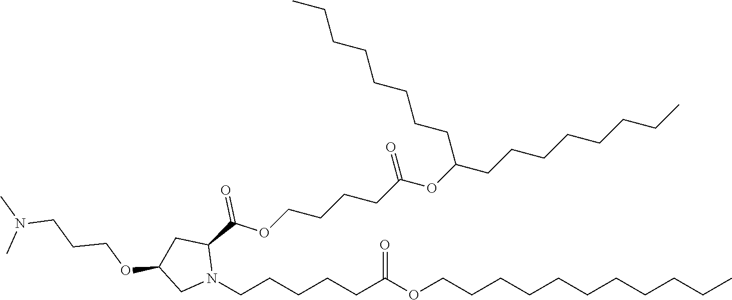 [SMILES: CCCCCCCCCCCOC(=O)CCCCCN1C[C@@H](OCCCN(C)C)C[C@H]1C(=O)OCCCCC(=O)OC(CCCCCCCC)CCCCCCCC] | 5-(heptadecan-9-yloxy)- 5-oxopentyl (2S,4S)-4- (3- (dimethylamino)propoxy)- 1-(6-oxo-6- (undecyloxy)hexyl)pyrro- lidine-2-carboxylate |
| 2423 | 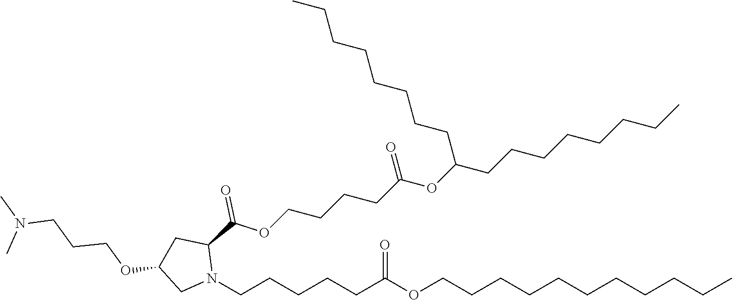 [SMILES: CCCCCCCCCCCOC(=O)CCCCCN1C[C@H](OCCCN(C)C)C[C@H]1C(=O)OCCCCC(=O)OC(CCCCCCCC)CCCCCCCC] | 5-(heptadecan-9-yloxy)-5- oxopentyl (2S,4R)-4-(3- (dimethylamino)propoxy)-1- (6-oxo-6- (undecyloxy)hexyl)pyrrolidine- 2-carboxylate |
| 2429 | 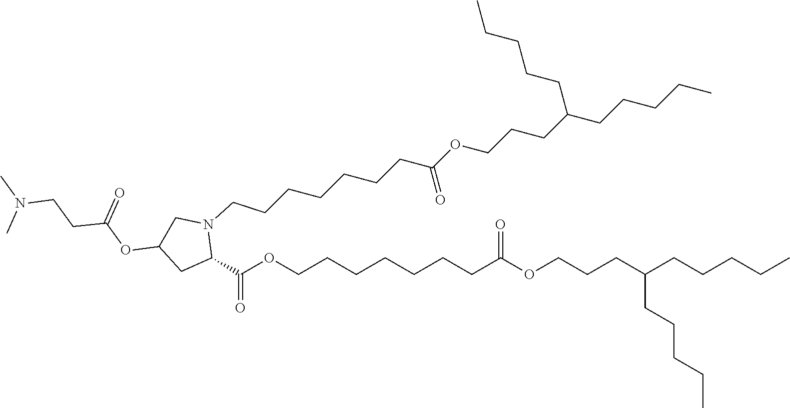 [SMILES: CCCCCC(CCCCC)CCCOC(=O)CCCCCCCOC(=O)[C@@H]1CC(OC(=O)CCN(C)C)CN1CCCCCCCC(=O)OCCCC(CCCCC)CCCCC] | 8-oxo-8-((4- pentylnonyl)oxy)octyl (2S)-4- ((3- (dimethylamino)propanoyl)oxy)- 1-(8-oxo-8-((4- pentylnonyl)oxy)octyl)pyrrolidine- 2-carboxylate |
| 2430 | 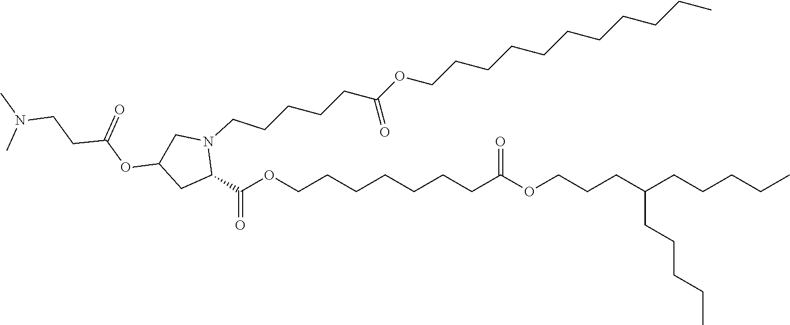 [SMILES: CCCCCCCCCCCOC(=O)CCCCCN1CC(OC(=O)CCN(C)C)C[C@H]1C(=O)OCCCCCCCC(=O)OCCCC(CCCCC)CCCCC] | 8-oxo-8-((4- pentylnonyl)oxy)octyl (2S)-4- ((3- (dimethylamino)propanoyl)oxy)- 1-(6-oxo-6- (undecyloxy)hexyl)pyrrolidine- 2-carboxylate |
| 2431 | 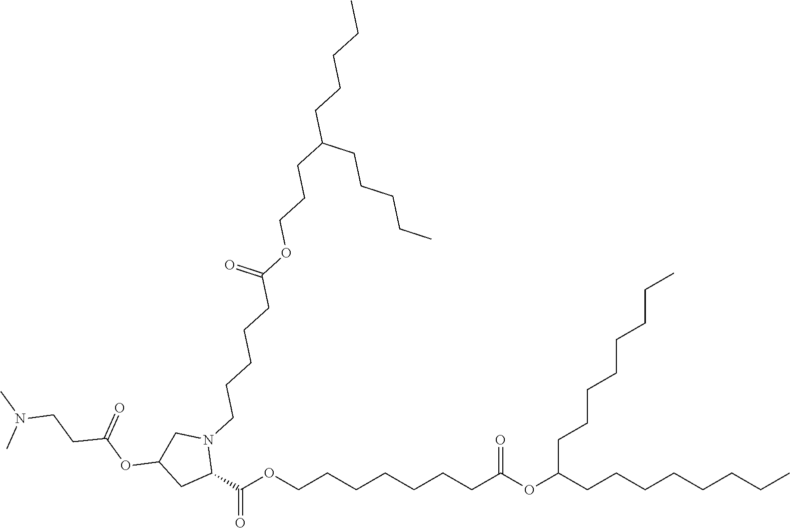 [SMILES: CCCCCCCCC(CCCCCCCC)OC(=O)CCCCCCCOC(=O)[C@@H]1CC(OC(=O)CCN(C)C)CN1CCCCCC(=O)OCCCC(CCCCC)CCCCC] | 8-(heptadecan-9-yloxy)-8- oxooctyl (2S)-4-((3- (dimethylamino)propanoyl)oxy)- 1-(6-oxo-6-((4- pentylnonyl)oxy)hexyl)pyrrolidine- 2-carboxylate |
| 2438 | 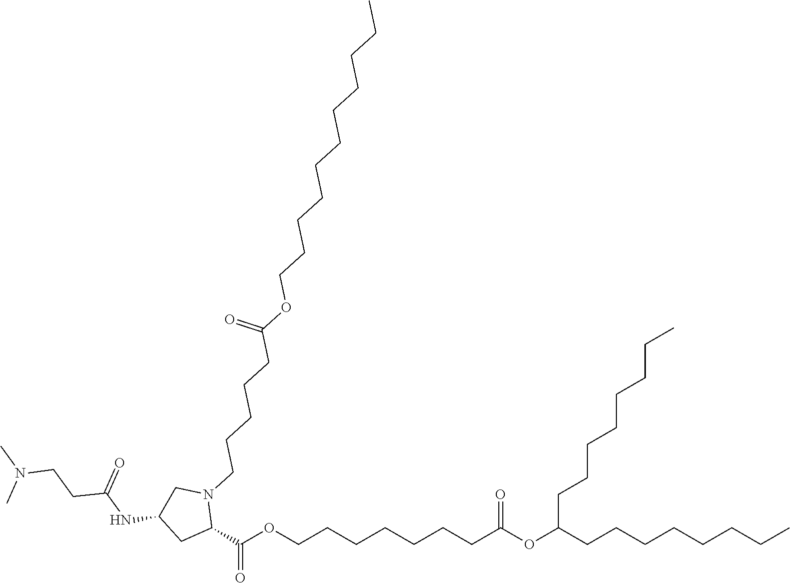 [SMILES: CCCCCCCCCCCOC(=O)CCCCCN1C[C@@H](NC(=O)CCN(C)C)C[C@H]1C(=O)OCCCCCCCC(=O)OC(CCCCCCCC)CCCCCCCC] | 8-(heptadecan-9-yloxy)-8- oxooctyl (2S,4S)-4-((3- (dimethylamino)propyl)amino)- 1-(6-oxo-6- (undecyloxy)hexyl)pyrrolidine- 2-carboxylate |
| 2451 | 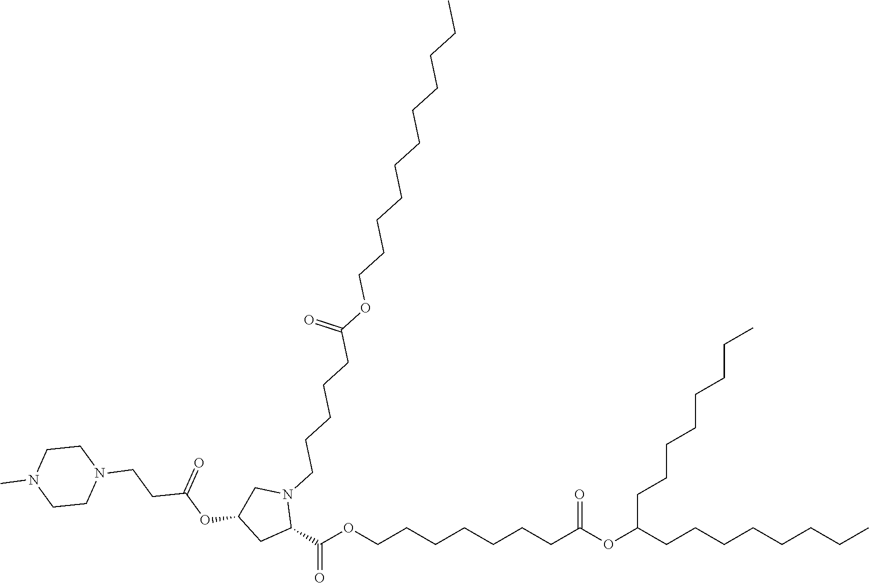 [SMILES: CCCCCCCCCCCOC(=O)CCCCCN1C[C@@H](OC(=O)CCN2CCN(C)CC2)C[C@H]1C(=O)OCCCCCCCC(=O)OC(CCCCCCCC)CCCCCCCC] | 8-(heptadecan-9-yloxy)-8- oxooctyl (2S,4S)-4-((3-(4- methylpiperazin-1- yl)propanoyl)oxy)-1-(6-oxo-6- (undecyloxy)hexyl)pyrrolidine- 2-carboxylate |
| 2455 | 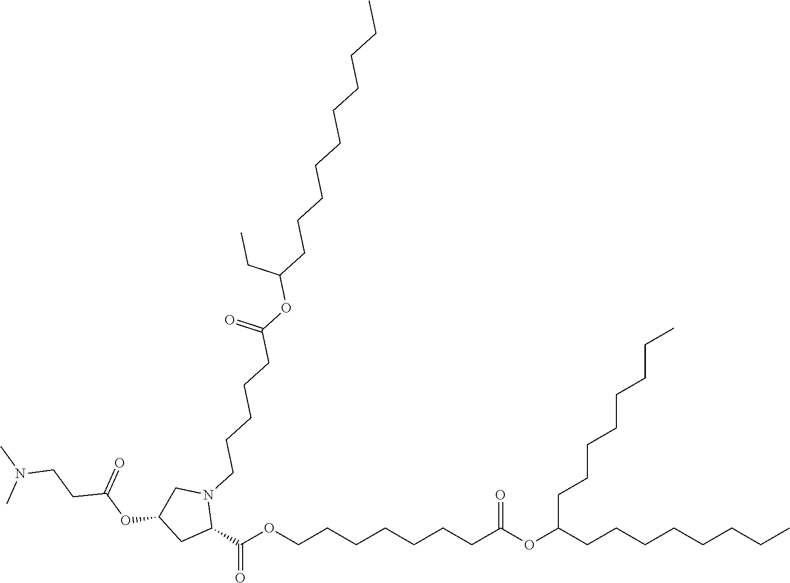 [SMILES: CCCCCCCCCCC(CC)OC(=O)CCCCCN1C[C@@H](OC(=O)CCN(C)C)C[C@H]1C(=O)OCCCCCCCC(=O)OC(CCCCCCCC)CCCCCCCC] | 8-(heptadecan-9-yloxy)-8- oxooctyl (2S,4S)-4-((3- (dimethylamino)propanoyl)oxy)- 1-(6-oxo-6-(tridecan-3- yloxy)hexyl)pyrrolidine-2- carboxylate |
| 1 | 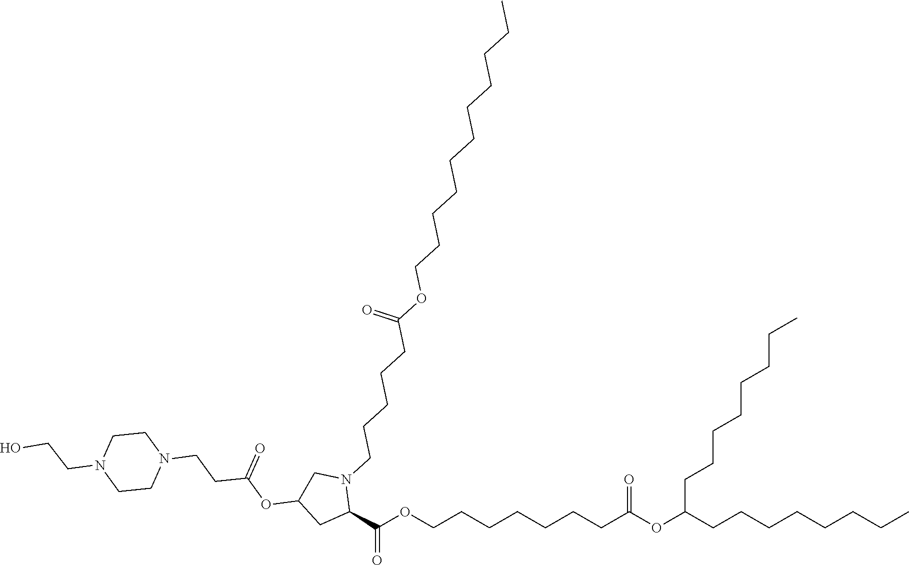 [SMILES: CCCCCCCCCCCOC(=O)CCCCCN1CC(OC(=O)CCN2CCN(CCO)CC2)C[C@@H]1C(=O)OCCCCCCCC(=O)OC(CCCCCCCC)CCCCCCCC] | 8-(heptadecan-9-yloxy)-8- oxooctyl (2S)-4-((3-(4-(2- hydroxyethyl)piperazin-1- yl)propanoyl)oxy)-1-(6-oxo-6- (undecyloxy)hexyl)pyrrolidine- 2-carboxylate |
| 2 | 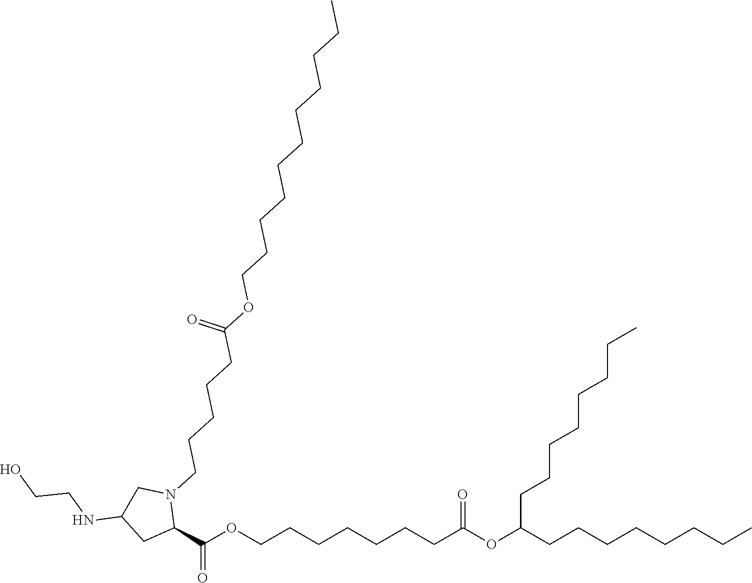 [SMILES: CCCCCCCCCCCOC(=O)CCCCCN1CC(NCCO)C[C@@H]1C(=O)OCCCCCCCC(=O)OC(CCCCCCCC)CCCCCCCC] | 8-(heptadecan-9-yloxy)-8- oxooctyl (2S)-4-((2- hydroxyethyl)amino)-1-(6- oxo-6- (undecyloxy)hexyl)pyrrolidine- 2-carboxylate |
| 3 | 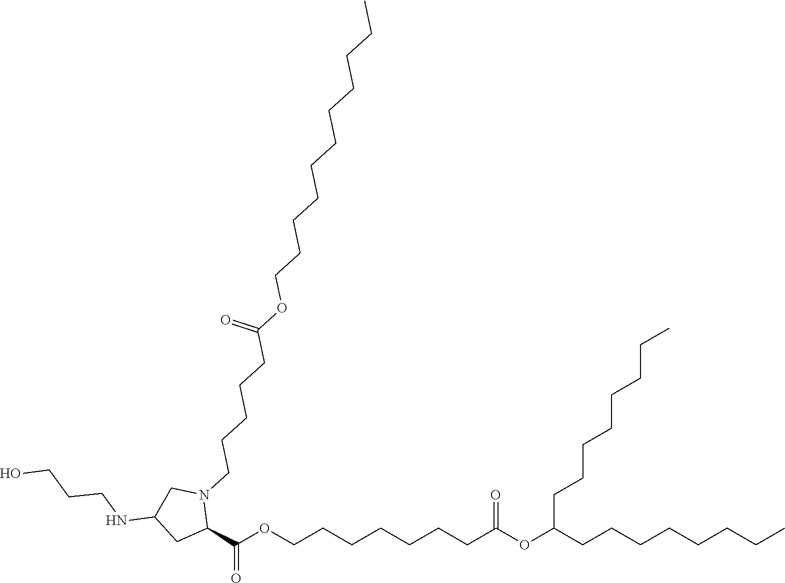 [SMILES: CCCCCCCCCCCOC(=O)CCCCCN1CC(NCCCO)C[C@@H]1C(=O)OCCCCCCCC(=O)OC(CCCCCCCC)CCCCCCCC] | 8-(heptadecan-9-yloxy)-8- oxooctyl (2S)-4-((3- hydroxypropyl)amino)-1-(6- oxo-6- (undecyloxy)hexyl)pyrrolidine- 2-carboxylate |
| 4 | 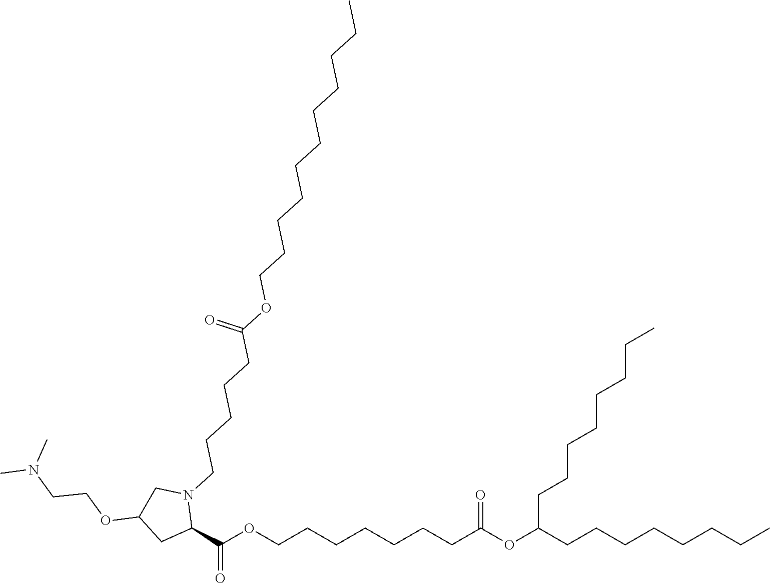 [SMILES: CCCCCCCCCCCOC(=O)CCCCCN1CC(OCCN(C)C)C[C@@H]1C(=O)OCCCCCCCC(=O)OC(CCCCCCCC)CCCCCCCC] | 8-(heptadecan-9-yloxy)-8- oxooctyl (2S)-4-(2- (dimethylamino)ethoxy)-1-(6- oxo-6- (undecyloxy)hexyl)pyrrolidine- 2-carboxylate |
| 5 | 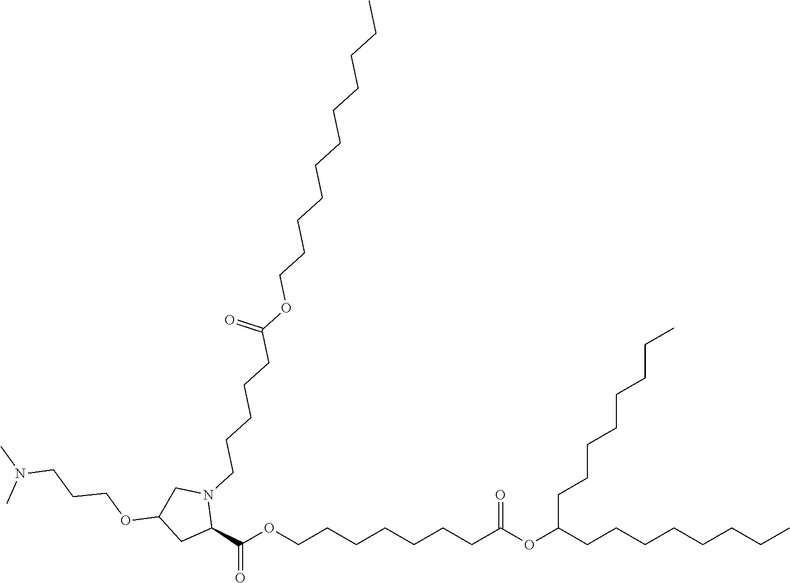 [SMILES: CCCCCCCCCCCOC(=O)CCCCCN1CC(OCCCN(C)C)C[C@@H]1C(=O)OCCCCCCCC(=O)OC(CCCCCCCC)CCCCCCCC] | 8-(heptadecan-9-yloxy)-8- oxooctyl (2S)-4-(3- (dimethylamino)propoxy)-1- (6-oxo-6- (undecyloxy)hexyl)pyrrolidine- 2-carboxylate |
| 6 | 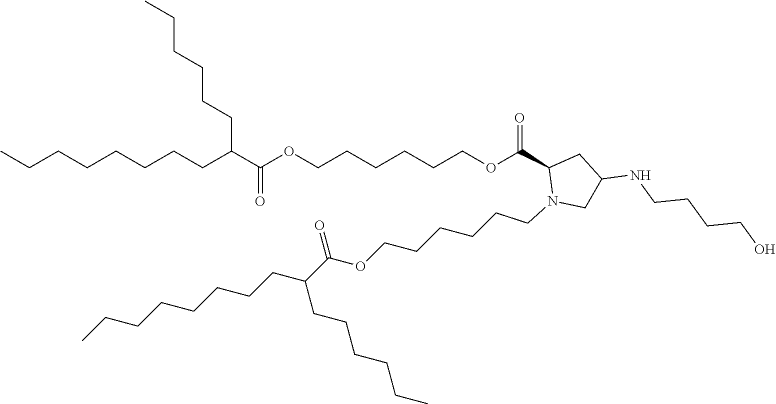 [SMILES: CCCCCCCCC(CCCCCC)C(=O)OCCCCCCOC(=O)[C@H]1CC(NCCCCO)CN1CCCCCCOC(=O)C(CCCCCC)CCCCCCCC] | 6-((2- hexyldecanoyl)oxy)hexyl (2S)-1-(6-((2- hexyldecanoyl)oxy)hexyl)-4- ((4- hydroxybutyl)amino)pyrrolidine- 2-carboxylate |
| 7 | 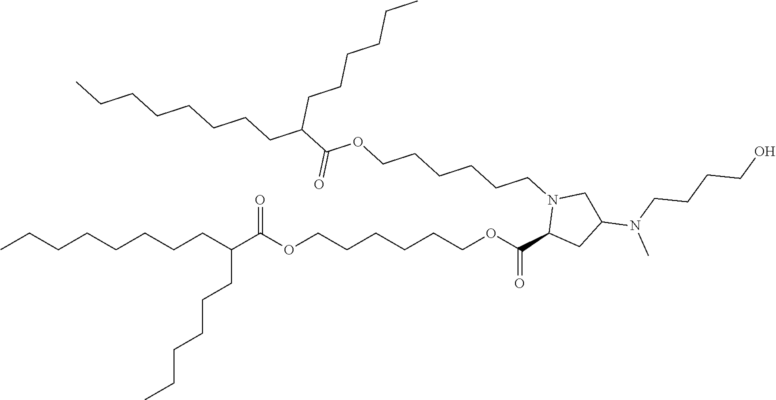 [SMILES: CCCCCCCCC(CCCCCC)C(=O)OCCCCCCOC(=O)[C@@H]1CC(N(C)CCCCO)CN1CCCCCCOC(=O)C(CCCCCC)CCCCCCCC] | 6-((2- hexyldecanoyl)oxy)hexyl (2S)-1-(6-((2- hexyldecanoyl)oxy)hexyl)-4- ((4- hydroxybutyl)(methyl)amino)pyrro- lidine-2-carboxylate |
| 8 | 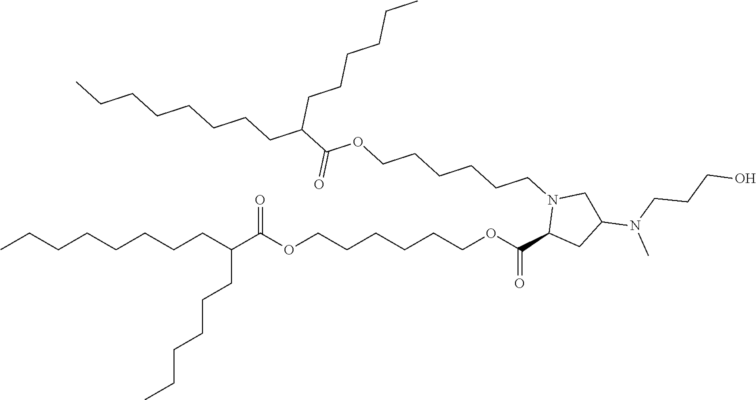 [SMILES: CCCCCCCCC(CCCCCC)C(=O)OCCCCCCOC(=O)[C@@H]1CC(N(C)CCCO)CN1CCCCCCOC(=O)C(CCCCCC)CCCCCCCC] | 6-((2- hexyldecanoyl)oxy)hexyl (2S)-1-(6-((2- hexyldecanoyl)oxy)hexyl)-4- ((3- hydroxypropyl)(methyl)amino) pyrrolidine-2-carboxylate |
| 9 | 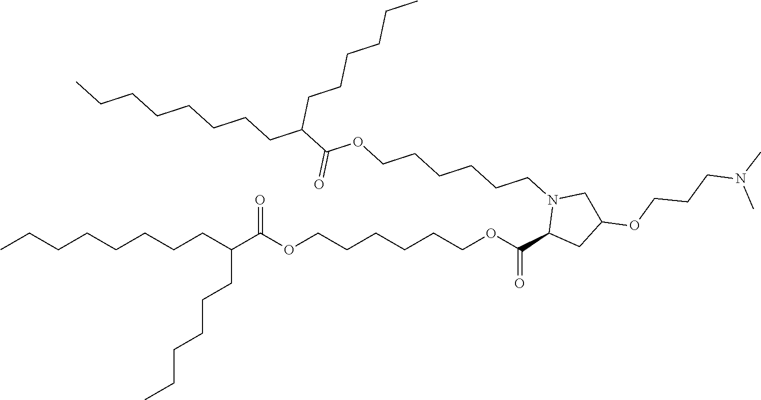 [SMILES: CCCCCCCCC(CCCCCC)C(=O)OCCCCCCOC(=O)[C@@H]1CC(OCCCN(C)C)CN1CCCCCCOC(=O)C(CCCCCC)CCCCCCCC] | 6-((2- hexyldecanoyl)oxy)hexyl (2S)-4-(3- (dimethylamino)propoxy)-1- (6-((2- hexyldecanoyl)oxy)hexyl)pyrro- lidine-2-carboxylate |
| 10 | 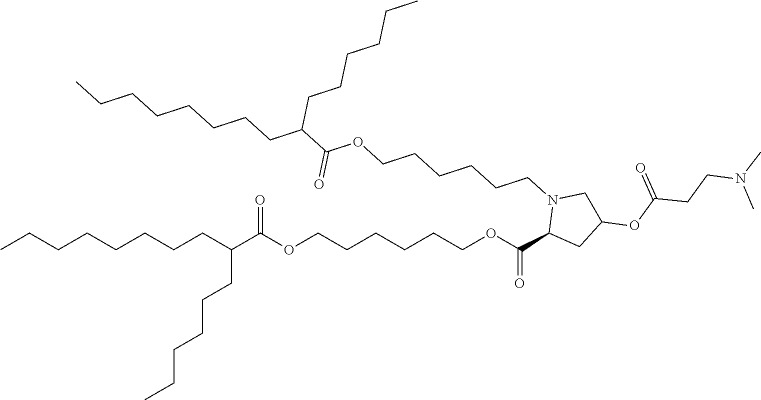 [SMILES: CCCCCCCCC(CCCCCC)C(=O)OCCCCCCOC(=O)[C@@H]1CC(OC(=O)CCN(C)C)CN1CCCCCCOC(=O)C(CCCCCC)CCCCCCCC] | 6-((2- hexyldecanoyl)oxy)hexyl (2S)-4-((3- (dimethylamino)propanoyl)oxy)- 1-(6-((2- hexyldecanoyl)oxy)hexyl)pyrro- lidine-2-carboxylate |
| 11 | 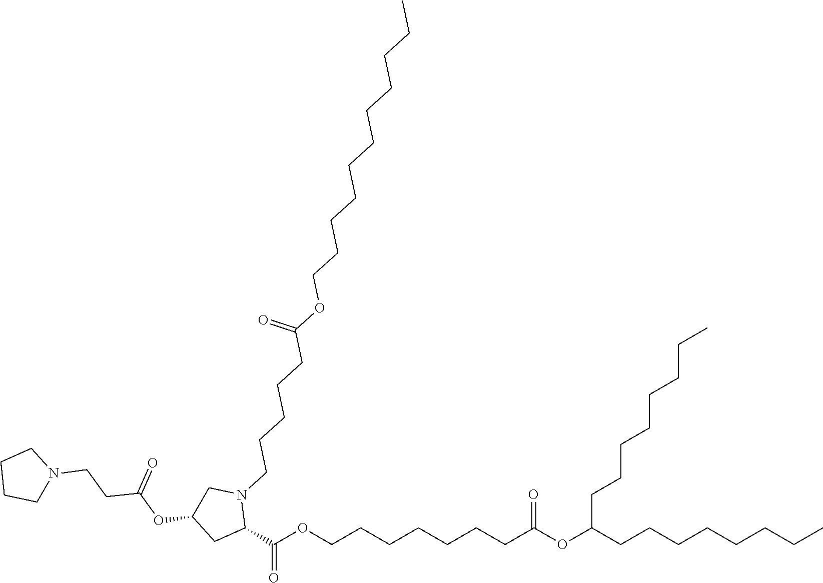 [SMILES: CCCCCCCCCCCOC(=O)CCCCCN1C[C@@H](OC(=O)CCN2CCCC2)C[C@H]1C(=O)OCCCCCCCC(=O)OC(CCCCCCCC)CCCCCCCC] | 8-(heptadecan-9-yloxy)-8- oxooctyl (2S,4S)-1-(6-oxo-6- (undecyloxy)hexyl)-4-((3- (pyrrolidin-1- yl)propanoyl)oxy)pyrrolidine- 2-carboxylate |
| 12. | 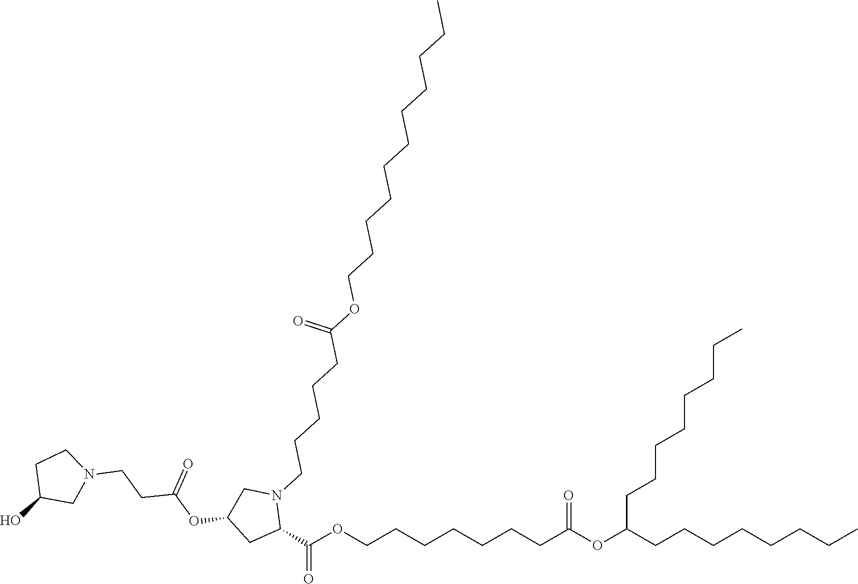 [SMILES: CCCCCCCCCCCOC(=O)CCCCCN1C[C@@H](OC(=O)CCN2CC[C@H](O)C2)C[C@H]1C(=O)OCCCCCCCC(=O)OC(CCCCCCCC)CCCCCCCC] | 8-(heptadecan-9-yloxy)-8- oxooctyl (2S,4S)-4-((3-((S)-3- hydroxypyrrolidin-1- yl)propanoyl)oxy)-1-(6-oxo-6- (undecyloxy)hexyl)pyrrolidine- 2-carboxylate |
| 13. | 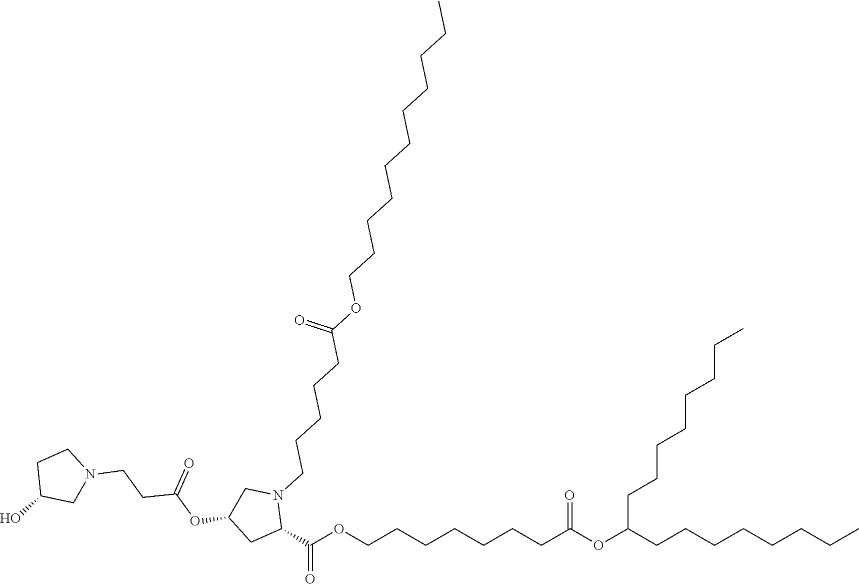 [SMILES: CCCCCCCCCCCOC(=O)CCCCCN1C[C@@H](OC(=O)CCN2CC[C@@H](O)C2)C[C@H]1C(=O)OCCCCCCCC(=O)OC(CCCCCCCC)CCCCCCCC] | 8-(heptadecan-9-yloxy)-8- oxooctyl (2S,4S)-4-((3-((R)-3- hydroxypyrrolidin-1- yl)propanoyl)oxy)-1-(6-oxo-6- (undecyloxy)hexyl)pyrrolidine- 2-carboxylate |
| 14. | 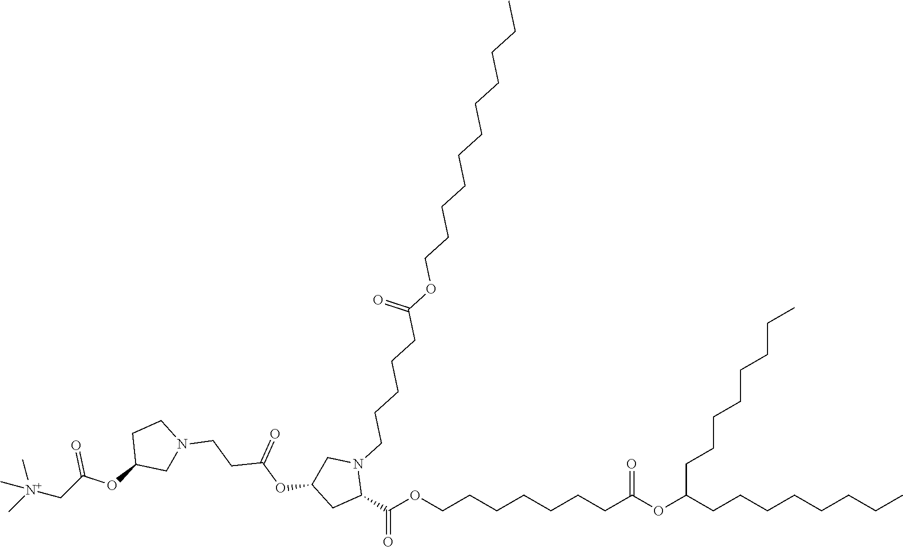 [SMILES: CCCCCCCCCCCOC(=O)CCCCCN1C[C@@H](OC(=O)CCN2CC[C@H](OC(=O)C[N+](C)(C)C)C2)C[C@H]1C(=O)OCCCCCCCC(=O)OC(CCCCCCCC)CCCCCCCC] | 2-(((S)-1-(3-(((3S,5S)-5-(((8- (heptadecan-9-yloxy)-8- oxooctyl)oxy)carbonyl)-1-(6- oxo-6- (undecyloxy)hexyl)pyrrolidin- 3-yl)oxy)-3- oxopropyl)pyrrolidin-3-yl)oxy)- N,N,N-trimethyl-2-oxoethan- 1-aminium |
| 15. | 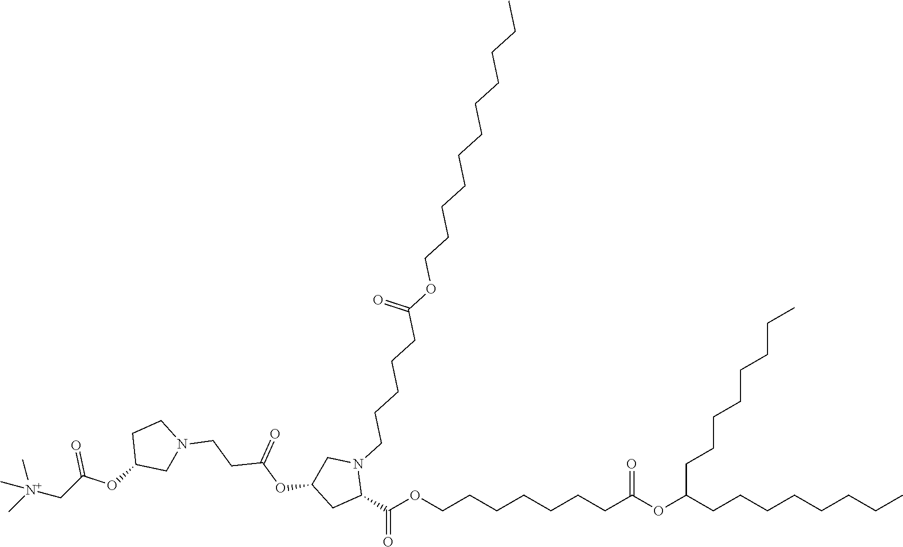 [SMILES: CCCCCCCCCCCOC(=O)CCCCCN1C[C@@H](OC(=O)CCN2CC[C@@H](OC(=O)C[N+](C)(C)C)C2)C[C@H]1C(=O)OCCCCCCCC(=O)OC(CCCCCCCC)CCCCCCCC] | 2-(((R)-1-(3-(((3S,5S)-5-(((8- (heptadecan-9-yloxy)-8- oxooctyl)oxy)carbonyl)-1-(6- oxo-6- (undecyloxy)hexyl)pyrrolidin- 3-yl)oxy)-3- oxopropyl)pyrrolidin-3-yl)oxy)- N,N,N-trimethyl-2-oxoethan- 1-aminium |
| 16. | 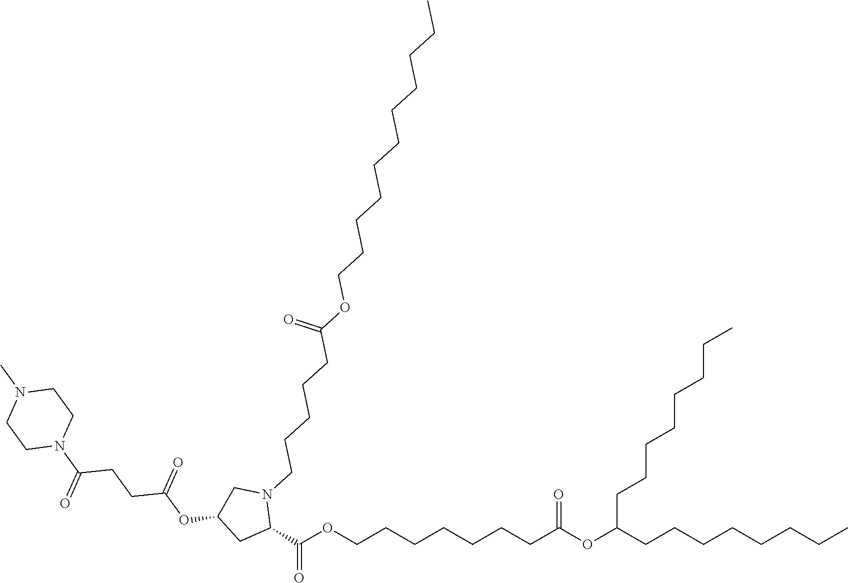 [SMILES: CCCCCCCCCCCOC(=O)CCCCCN1C[C@@H](OC(=O)CCC(=O)N2CCN(C)CC2)C[C@H]1C(=O)OCCCCCCCC(=O)OC(CCCCCCCC)CCCCCCCC] | 8-(heptadecan-9-yloxy)-8- oxooctyl (2S,4S)-4-((4-(4- methylpiperazin-1-yl)-4- oxobutanoyl)oxy)-1-(6-oxo-6- (undecyloxy)hexyl)pyrrolidine- 2-carboxylate |
| 17. | 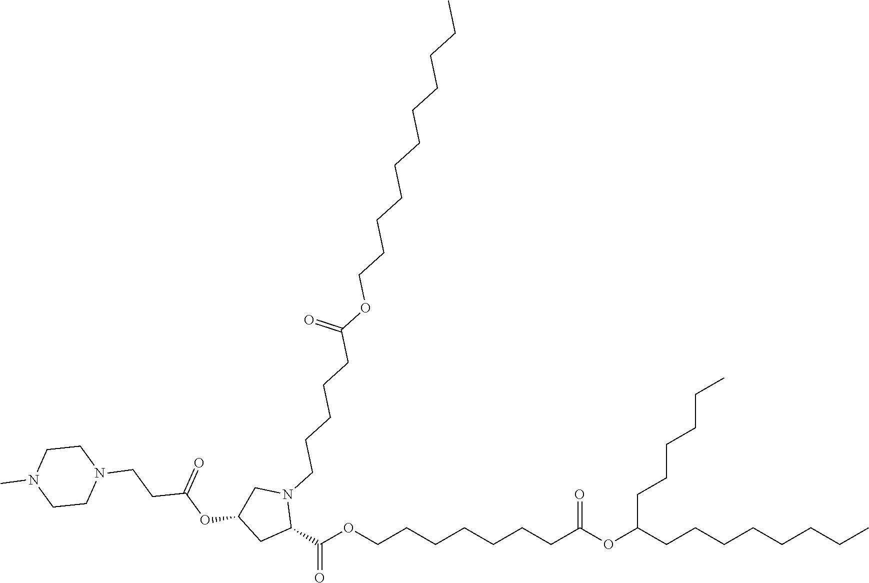 [SMILES: CCCCCCCCCCCOC(=O)CCCCCN1C[C@@H](OC(=O)CCN2CCN(C)CC2)C[C@H]1C(=O)OCCCCCCCC(=O)OC(CCCCCC)CCCCCCCC] | 8-oxo-8-(pentadecan-7- yloxy)octyl (2S,4S)-4-((3-(4- methylpiperazin-1- yl)propanoyl)oxy)-1-(6-oxo-6- (undecyloxy)hexyl)pyrrolidine- 2-carboxylate |
| 18. | 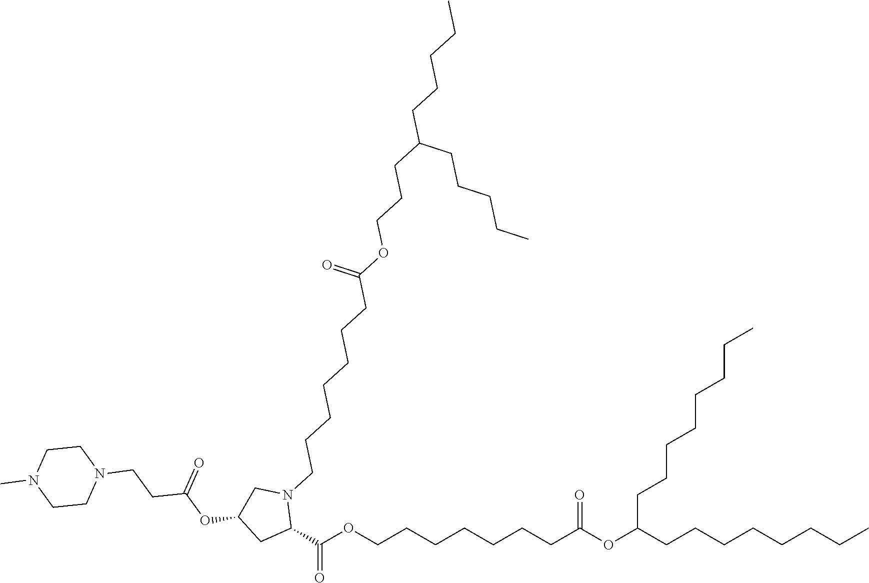 [SMILES: CCCCCCCCC(CCCCCCCC)OC(=O)CCCCCCCOC(=O)[C@@H]1C[C@H](OC(=O)CCN2CCN(C)CC2)CN1CCCCCCCC(=O)OCCCC(CCCCC)CCCCC] | 8-(heptadecan-9-yloxy)-8- oxooctyl (2S,4S)-4-((3-(4- methylpiperazin-1- yl)propanoyl)oxy)-1-(8-oxo-8- ((4- pentylnonyl)oxy)octyl)pyrrolidine- 2-carboxylate |
| 19. | 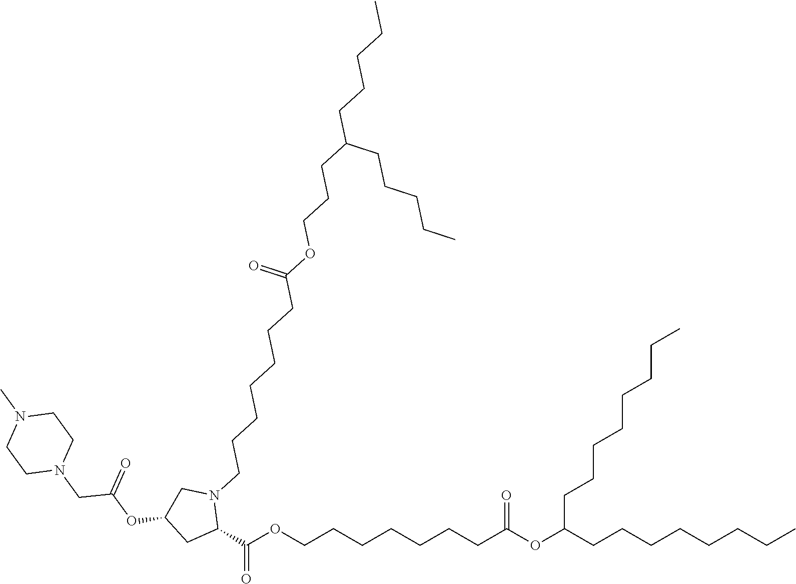 [SMILES: CCCCCCCCC(CCCCCCCC)OC(=O)CCCCCCCOC(=O)[C@@H]1C[C@H](OC(=O)CN2CCN(C)CC2)CN1CCCCCCCC(=O)OCCCC(CCCCC)CCCCC] | 8-(heptadecan-9-yloxy)-8- oxooctyl (2S,4S)-4-(2-(4- methylpiperazin-1- yl)acetoxy)-1-(8-oxo-8-((4- pentylnonyl)oxy)octyl)pyrrolidine- 2-carboxylate |
| 20. | 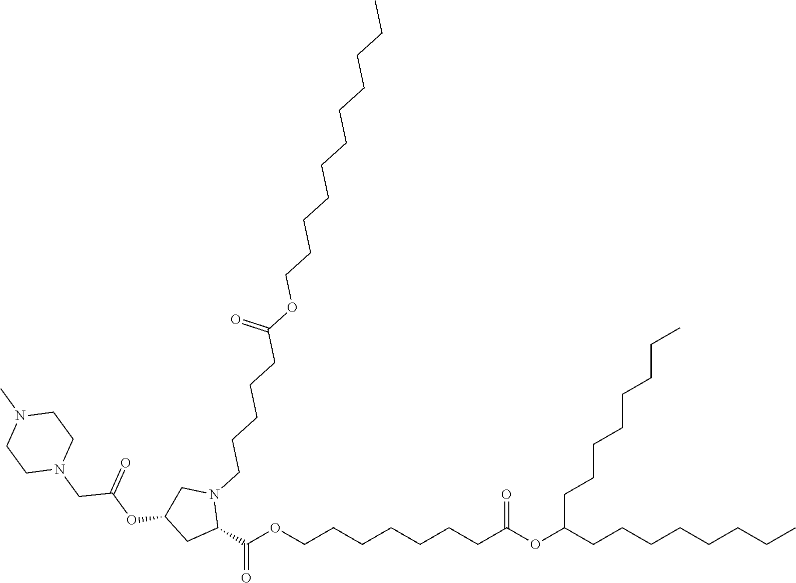 [SMILES: CCCCCCCCCCCOC(=O)CCCCCN1C[C@@H](OC(=O)CN2CCN(C)CC2)C[C@H]1C(=O)OCCCCCCCC(=O)OC(CCCCCCCC)CCCCCCCC] | 8-(heptadecan-9-yloxy)-8- oxooctyl (2S,4S)-4-(2-(4- methylpiperazin-1- yl)acetoxy)-1-(6-oxo-6- (undecyloxy)hexyl)pyrrolidine- 2-carboxylate |
| 21. | 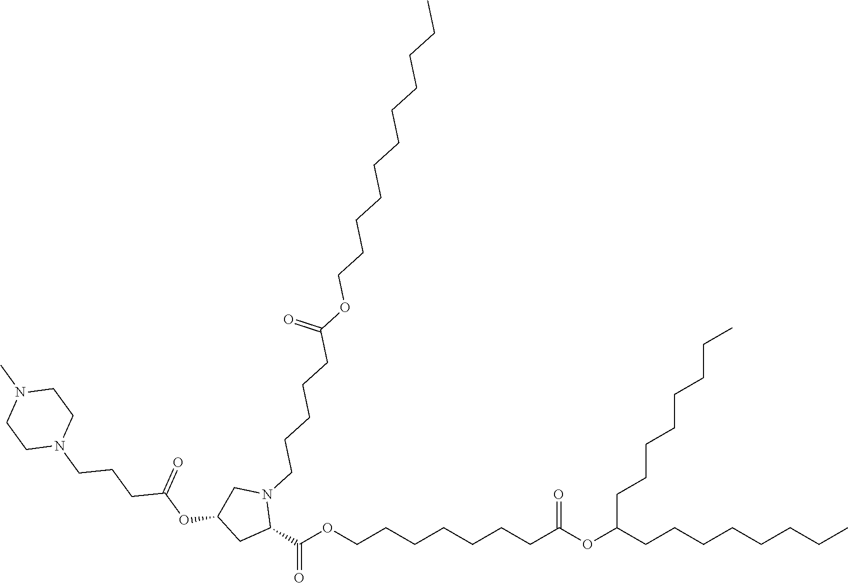 [SMILES: CCCCCCCCCCCOC(=O)CCCCCN1C[C@@H](OC(=O)CCCN2CCN(C)CC2)C[C@H]1C(=O)OCCCCCCCC(=O)OC(CCCCCCCC)CCCCCCCC] | 8-(heptadecan-9-yloxy)-8- oxooctyl (2S,4S)-4-((4-(4- methylpiperazin-1- yl)butanoyl)oxy)-1-(6-oxo-6- (undecyloxy)hexyl)pyrrolidine- 2-carboxylate |
| 22. | 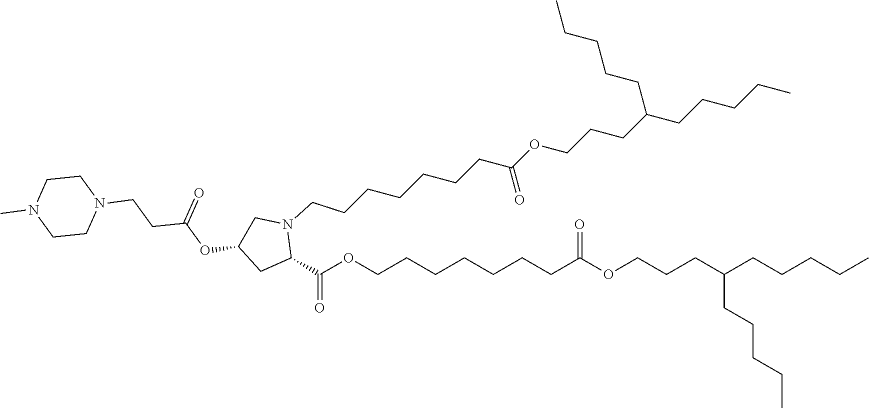 [SMILES: CCCCCC(CCCCC)CCCOC(=O)CCCCCCCOC(=O)[C@@H]1C[C@H](OC(=O)CCN2CCN(C)CC2)CN1CCCCCCCC(=O)OCCCC(CCCCC)CCCCC] | 8-oxo-8-((4- pentylnonyl)oxy)octyl (2S,4S)-4-((3-(4- methylpiperazin-1- yl)propanoyl)oxy)-1-(8-oxo-8- ((4- pentylnonyl)oxy)octyl)pyrrolidine- 2-carboxylate |
| 23. | 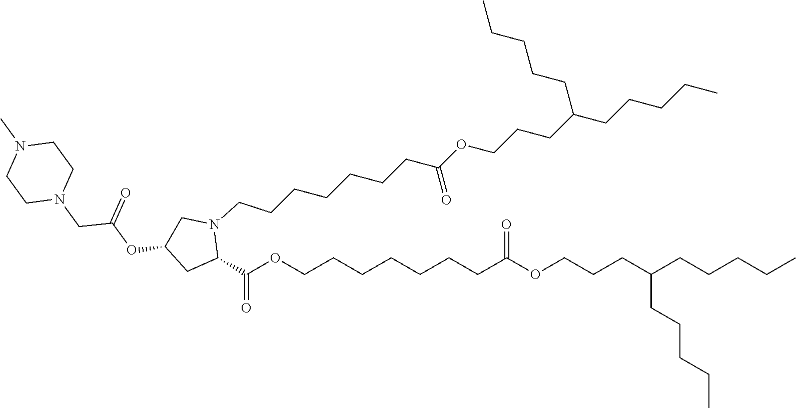 [SMILES: CCCCCC(CCCCC)CCCOC(=O)CCCCCCCOC(=O)[C@@H]1C[C@H](OC(=O)CN2CCN(C)CC2)CN1CCCCCCCC(=O)OCCCC(CCCCC)CCCCC] | 8-oxo-8-((4- pentylnonyl)oxy)octyl (2S,4S)-4-(2-(4- methylpiperazin-1- yl)acetoxy)-1-(8-oxo-8-((4- pentylnonyl)oxy)octyl)pyrrolidine- 2-carboxylate |
| 24. | 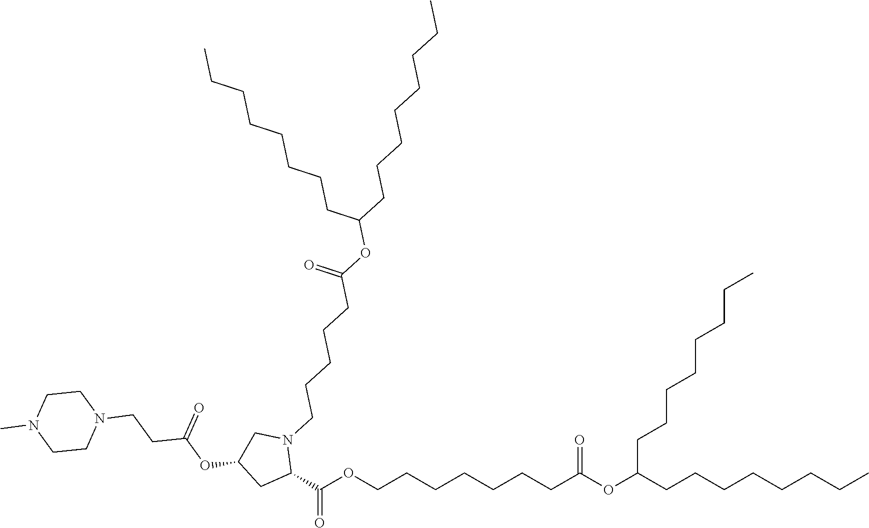 [SMILES: CCCCCCCCC(CCCCCCCC)OC(=O)CCCCCCCOC(=O)[C@@H]1C[C@H](OC(=O)CCN2CCN(C)CC2)CN1CCCCCC(=O)OC(CCCCCCCC)CCCCCCCC] | 8-(heptadecan-9-yloxy)-8- oxooctyl (2S,4S)-1-(6- (heptadecan-9-yloxy)-6- oxohexyl)-4-((3-(4- methylpiperazin-1- yl)propanoyl)oxy)pyrrolidine- 2-carboxylate |
| 25. | 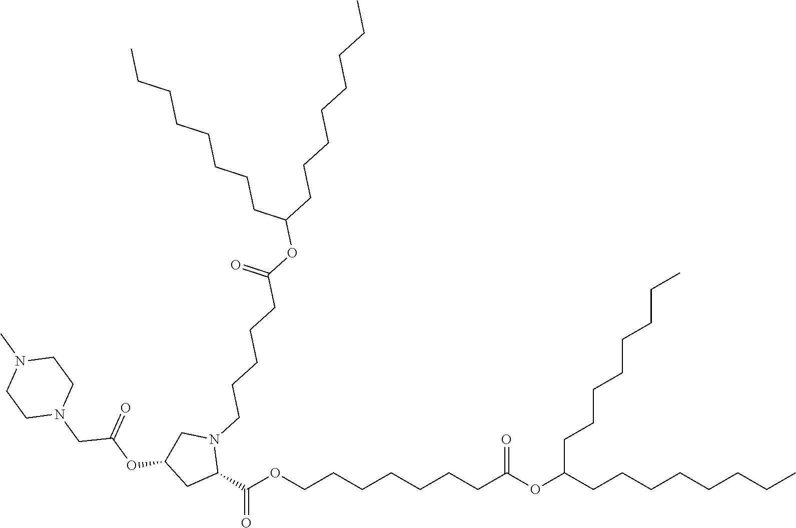 [SMILES: CCCCCCCCC(CCCCCCCC)OC(=O)CCCCCCCOC(=O)[C@@H]1C[C@H](OC(=O)CN2CCN(C)CC2)CN1CCCCCC(=O)OC(CCCCCCCC)CCCCCCCC] | 8-(heptadecan-9-yloxy)-8- oxooctyl (2S,4S)-1-(6- (heptadecan-9-yloxy)-6- oxohexyl)-4-(2-(4- methylpiperazin-1- yl)acetoxy)pyrrolidine-2- carboxylate |
| 26. | 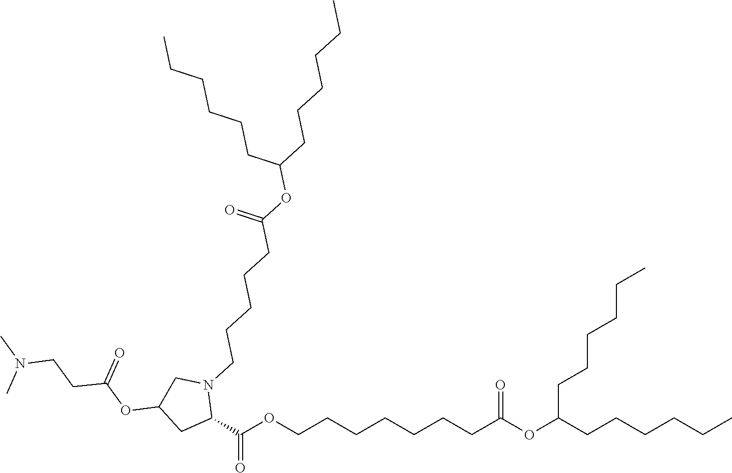 [SMILES: CCCCCCC(CCCCCC)OC(=O)CCCCCCCOC(=O)[C@@H]1CC(OC(=O)CCN(C)C)CN1CCCCCC(=O)OC(CCCCCC)CCCCCC] | 8-oxo-8-(tridecan-7- yloxy)octyl (2S)-4-((3- (dimethylamino)propanoyl)oxy)- 1-(6-oxo-6-(tridecan-7- yloxy)hexyl)pyrrolidine-2- carboxylate |
| 27. | 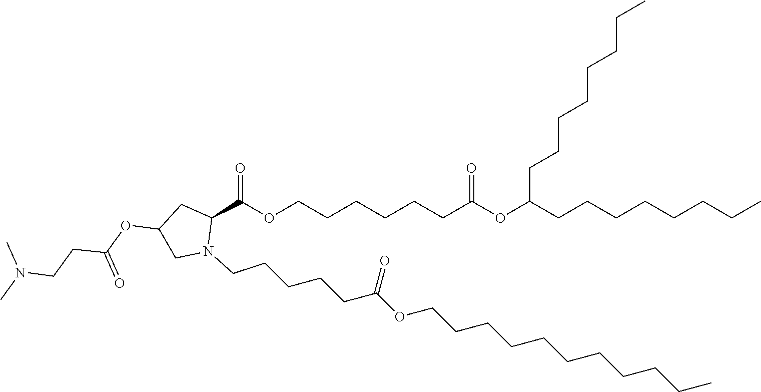 [SMILES: CCCCCCCCCCCOC(=O)CCCCCN1CC(OC(=O)CCN(C)C)C[C@H]1C(=O)OCCCCCCC(=O)OC(CCCCCCCC)CCCCCCCC] | 7-(heptadecan-9-yloxy)-7- oxoheptyl (2S)-4-((3- (dimethylamino)propanoyl)oxy)- 1-(6-oxo-6- (undecyloxy)hexyl)pyrrolidine- 2-carboxylate |
| 28. | 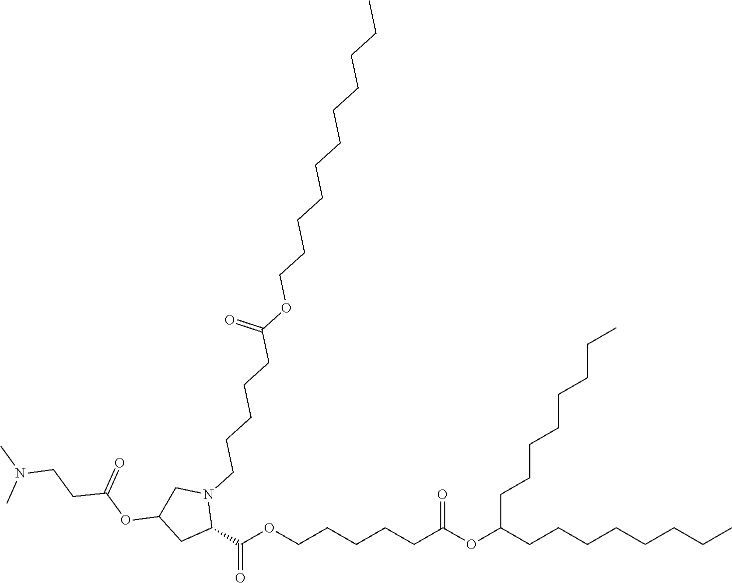 [SMILES: CCCCCCCCCCCOC(=O)CCCCCN1CC(OC(=O)CCN(C)C)C[C@H]1C(=O)OCCCCCC(=O)OC(CCCCCCCC)CCCCCCCC] | 6-(heptadecan-9-yloxy)-6- oxohexyl (2S)-4-((3- (dimethylamino)propanoyl)oxy)- 1-(6-oxo-6- (undecyloxy)hexyl)pyrrolidine- 2-carboxylate |
| 29. | 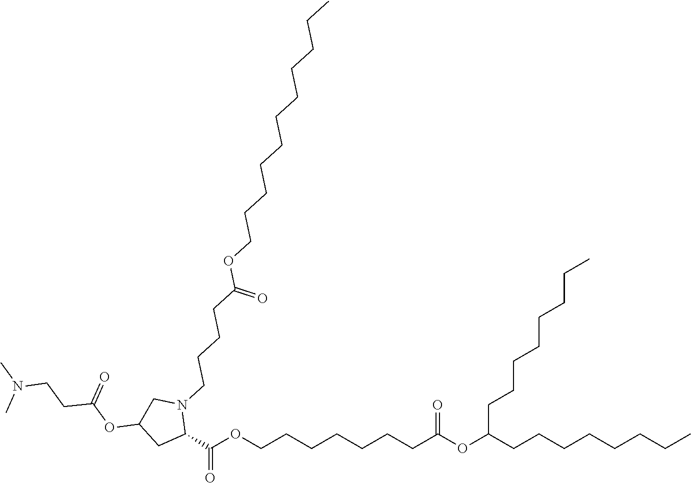 [SMILES: CCCCCCCCCCCOC(=O)CCCCN1CC(OC(=O)CCN(C)C)C[C@H]1C(=O)OCCCCCCCC(=O)OC(CCCCCCCC)CCCCCCCC] | 8-(heptadecan-9-yloxy)-8- oxooctyl (2S)-4-((3- (dimethylamino)propanoyl)oxy)- 1-(5-oxo-5- (undecyloxy)pentyl)pyrrolidine- 2-carboxylate |
| 30. | 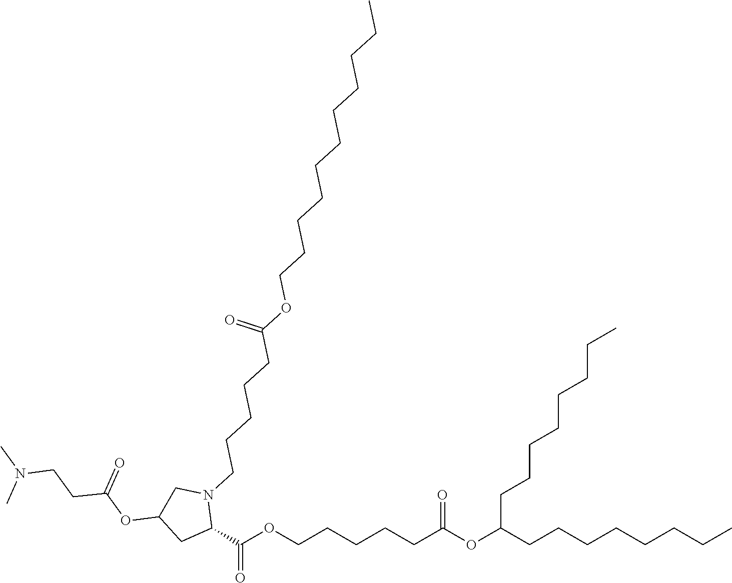 [SMILES: CCCCCCCCCCCOC(=O)CCCCCN1CC(OC(=O)CCN(C)C)C[C@H]1C(=O)OCCCCCC(=O)OC(CCCCCCCC)CCCCCCCC] | 6-(heptadecan-9-yloxy)-6- oxohexyl (2S)-4-((3- (dimethylamino)propanoyl)oxy)- 1-(6-oxo-6- (undecyloxy)hexyl)pyrrolidine- 2-carboxylate |
| 31. | 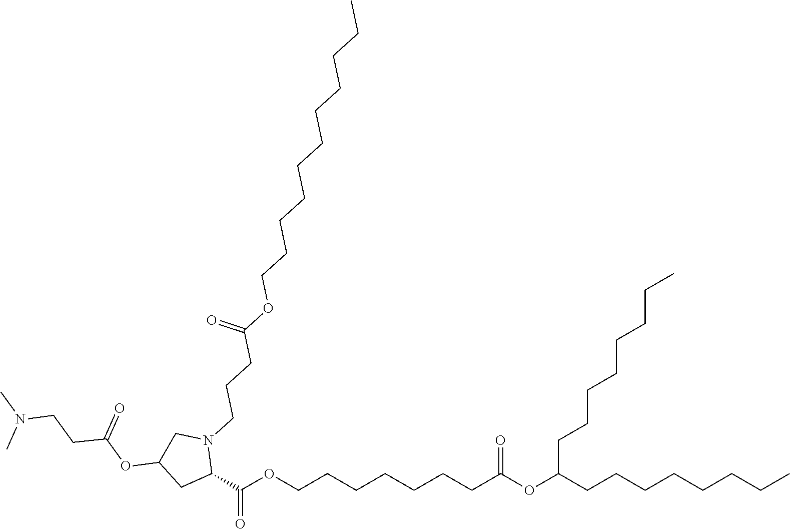 [SMILES: CCCCCCCCCCCOC(=O)CCCN1CC(OC(=O)CCN(C)C)C[C@H]1C(=O)OCCCCCCCC(=O)OC(CCCCCCCC)CCCCCCCC] | 8-(heptadecan-9-yloxy)-8- oxooctyl (2S)-4-((3- (dimethylamino)propanoyl)oxy)- 1-(4-oxo-4- (undecyloxy)butyl)pyrrolidine- 2-carboxylate |
| 32. | 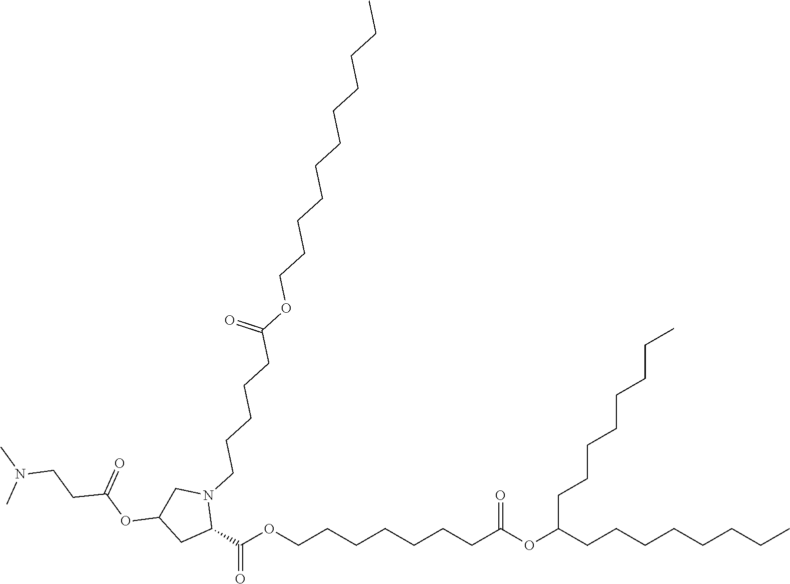 [SMILES: CCCCCCCCCCCOC(=O)CCCCCN1CC(OC(=O)CCN(C)C)C[C@H]1C(=O)OCCCCCCCC(=O)OC(CCCCCCCC)CCCCCCCC] | 8-(heptadecan-9-yloxy)-8- oxooctyl (2S)-4-((3- (dimethylamino)propanoyl)oxy)- 1-(6-(nonyloxy)-6- oxohexyl)pyrrolidine-2- carboxylate |
| 33. | 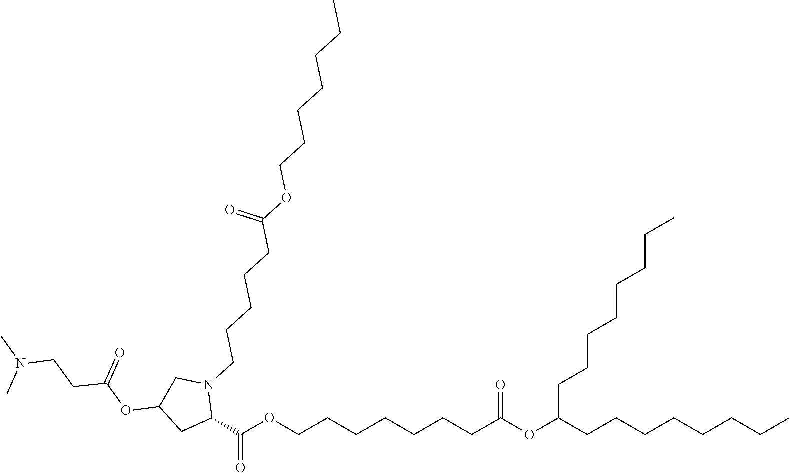 [SMILES: CCCCCCCCC(CCCCCCCC)OC(=O)CCCCCCCOC(=O)[C@@H]1CC(OC(=O)CCN(C)C)CN1CCCCCC(=O)OCCCCCCC] | 8-(heptadecan-9-yloxy)-8- oxooctyl (2S)-4-((3- (dimethylamino)propanoyl)oxy)- 1-(6-(heptyloxy)-6- oxohexyl)pyrrolidine-2- carboxylate |

Ionizable Lipid Compounds iii)

In some embodiments, the ionizable lipid is represented by formula

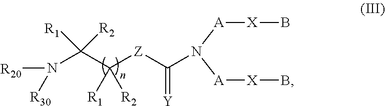

pharmaceutically acceptable salts thereof, and stereoisomers of any of the foregoing, wherein:
R₂₀ and R₃₀ are each independently H, C₁-C₅ branched or unbranched alkyl, or C₂-C₅ branched or unbranched alkenyl, or
R₂₀ and R₃₀ together with the adjacent N atom form a 3 to 7 membered cyclic ring, optionally substituted with R^{a};
R^{a} is H, C₁-C₃ branched or unbranched alkyl, C₂-C₃ branched or unbranched alkenyl, halogen, OH, or SH;
each R₁ and each R₂ is independently H, C₁-C₃ branched or unbranched alkyl, C₂-C₃ branched or unbranched alkenyl, OH, halogen, SH, or NR₁₀R₁₁, or
R₁ and R₂ are taken together to form a cyclic ring;
each R₁₀ and R₁₁ is independently H, C₁-C₃ branched or unbranched alkyl, C₂-C₃ branched or unbranched alkenyl, or
R₁₀ and R₁₁ are taken together to form a heterocyclic ring;
n is 0, 1, 2, 3 or 4;
Y is O or S;
Z is absent, O, S, or N(R₁₂), wherein each R₁₂ is independently H, C₁-C₇ branched or unbranched alkyl, or C₂-C₇ branched or unbranched alkenyl, provided that when Z is not absent, the adjacent R₁ and R₂ cannot be OH, NR₁₀R₁₁, or SH;
each A is each independently C₁-C₁₆ branched or unbranched alkyl, or C₂-C₁₆ branched or unbranched alkenyl, optionally interrupted with one or more heteroatoms or optionally substituted with OH, SH, or halogen;
each B is each independently C₁-C₁₆ branched or unbranched alkyl, or C₂-C₁₆ branched or unbranched alkenyl, optionally interrupted with one or more heteroatoms or optionally substituted with OH, SH, or halogen;
each X is independently a biodegradable moiety.

In some embodiments, R₂₀ and R₃₀ are each independently H or C₁-C₃ branched or unbranched alkyl. In some embodiments, R₂₀ and R₃₀ together with the adjacent N atom form a 3 to 7 membered cyclic ring, optionally substituted with R^{a}. In some embodiments, R^{a} is H, C₁-C₃ branched or unbranched alkyl or OH. In one embodiment, R^{a} is H or OH.

In some embodiments, Z is absent, S, O, or NH. In some embodiments, n is 0, 1, or 2.

In some embodiments, the ionizable lipid is represented by formula (V):

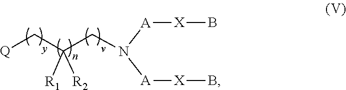

pharmaceutically acceptable salts thereof, and stereoisomers of any of the foregoing, wherein
R₁ is H, C₁-C₃ branched or unbranched alkyl, C₂-C₃ branched or unbranched alkenyl, OH, halogen, SH, or NR₁₀R₁₁, and
R₂ is H, OH, halogen, SH, or NR₁₀R₁₁, or
R₁ and R₂ are taken together to form a cyclic ring;
R₁₀ and R₁₁ are each independently H or C₁-C₃ alkyl, or R₁₀ and R₁₁ are taken together to form a heterocyclic ring;
Q is OH or —(OCH₂CH₂)ᵤNR₂₀R₃₀,
R₂₀ and R₃₀ are each independently H, C₁-C₅ branched or unbranched alkyl, or C₂-C₅ branched or unbranched alkenyl, or
R₂₀ and R₃₀ together with the adjacent N atom form a 3 to 7 membered cyclic ring optionally substituted with R^{a};
R^{a} is H, C₁-C₃ branched or unbranched alkyl, C₂-C₃ branched or unbranched alkenyl, halogen, OH, or SH;
v is 0, 1, 2, 3, or 4;
y is 0, 1, 2, 3, or 4;
each A is independently C₁-C₁₆ branched or unbranched alkyl or C₁-C₁₆ branched or unbranched alkenyl, optionally interrupted with one or more heteroatoms or optionally substituted with OH, SH, or halogen;
each B is each independently C₁-C₁₆ branched or unbranched alkyl or C₂-C₁₆ branched or unbranched alkenyl, optionally interrupted with one or more heteroatoms or optionally substituted with OH, SH, or halogen; and
each X is independently a biodegradable moiety.

In some embodiments, the disclosure relates to ionizable lipids of one of the following formulas:

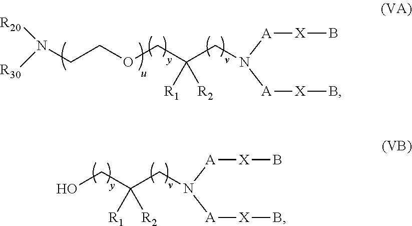

wherein: u is 0, 1, 2, 3, 4, 5, 6, 7, or 8; v is 0, 1, 2, 3, or 4; and y is 0, 1, 2, 3, or 4. Other variables are defined as in formulas Ill) and V) above.

In some embodiments, in the above formulas, X is —OC(O)—, —C(O)O—, —N(R⁷)C(O)—, —C(O—R₁₃)—O—, —C(O)O(CH₂)ₛ—, —OC(O)(CH₂)ₛ—, —C(O)N(R⁷)(CH₂)ₛ—, —N(R⁷)C(O)(CH₂)ₛ—, —C(O—R₁₃)—O—(CH₂)ₛ—, wherein each R⁷ is independently H, alkyl, alkenyl, cycloalkyl, hydroxyalkyl, or aminoalkyl, each R₁₃ is independently C₃-C₁₀ alkyl, and each s is independently 0-16. In some embodiments, X is —OC(O)—, —C(O)O—, —C(O)O(CH₂)ₛ—, or —OC(O)(CH₂)ₛ—. In some embodiments, s is 0, 1, 2, 3, 4, 5, 6, 7, 8, 9, or 10.

More embodiments of the ionizable lipid of formula (III) or (V), in the Ionizable lipid compounds group iii), may be found in POT Application No. PCT/US22/50111, filed on Nov. 16, 2022, the content of which is incorporated herein by reference in its entirety. In particular, all the ionizable lipids of formulas (IO)—(VIIO) and formulas (I)—(VIID) of PCT Application No. PCT/US22/50111 are suitable for use as the ionizable lipids in this disclosure, and are incorporated herein by reference in its entirety.

Certain exemplary ionizable lipid compounds disclosed herein are set forth in Table Ill below.

TABLE III
Exemplary ionizable lipid compounds.

| Lipid | | |
|---|---|---|
| No. | Structure | Name |
| 2140 | 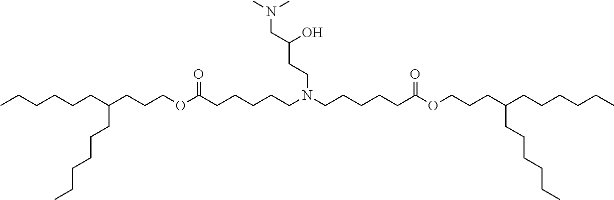 [SMILES: CCCCCCC(CCCCCC)CCCOC(=O)CCCCCN(CCCCCC(=O)OCCCC(CCCCCC)CCCCCC)CCC(O)CN(C)C] | bis(4-hexyldecyl) 6,6′- ((4-(dimethylamino)-3- hydroxybutyl)azanediyl) dihexanoate |
| 2139 | 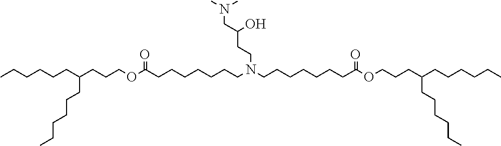 [SMILES: CCCCCCC(CCCCCC)CCCOC(=O)CCCCCCCN(CCCCCCCC(=O)OCCCC(CCCCCC)CCCCCC)CCC(O)CN(C)C] | bis(4-hexyldecyl) 8,8′- ((4-(dimethylamino)-3- hydroxybutyl)azanediyl) dioctanoate |
| 2138 | 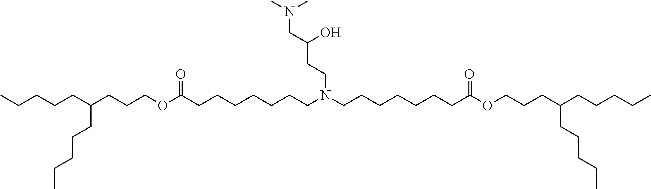 [SMILES: CCCCCC(CCCCC)CCCOC(=O)CCCCCCCN(CCCCCCCC(=O)OCCCC(CCCCC)CCCCC)CCC(O)CN(C)C] | bis(4-pentylnonyl) 8,8′- ((4-(dimethylamino)-3- hydroxybutyl)azanediyl) dioctanoate |
| 2142 | 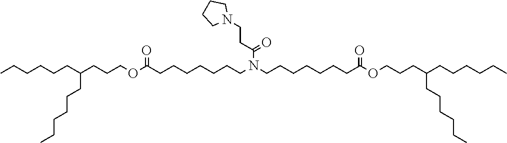 [SMILES: CCCCCCC(CCCCCC)CCCOC(=O)CCCCCCCN(CCCCCCCC(=O)OCCCC(CCCCCC)CCCCCC)C(=O)CCN1CCCC1] | bis(4-hexyldecyl) 8,8′- ((3-(pyrrolidin-1- yl)propanoyl)azanediyl) dioctanoate |
| 2146 | 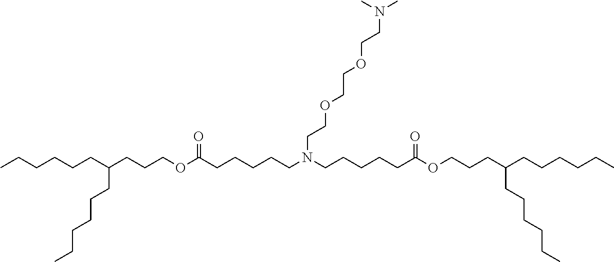 [SMILES: CCCCCCC(CCCCCC)CCCOC(=O)CCCCCN(CCCCCC(=O)OCCCC(CCCCCC)CCCCCC)CCOCCOCCN(C)C] | 4-hexyldecyl 11-(6-((4- hexyldecyl)oxy)-6- oxohexyl)-2-methyl-5,8- dioxa-2,11- diazaheptadecan-17- oate |
| 2145 | 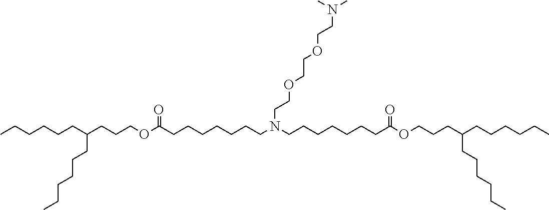 [SMILES: CCCCCCC(CCCCCC)CCCOC(=O)CCCCCCCN(CCCCCCCC(=O)OCCCC(CCCCCC)CCCCCC)CCOCCOCCN(C)C] | 4-hexyldecyl 11-(8-((4- hexyldecyl)oxy)-8- oxooctyl)-2-methyl-5,8- dioxa-2,11- diazanonadecan-19- oate |
| 2133 | 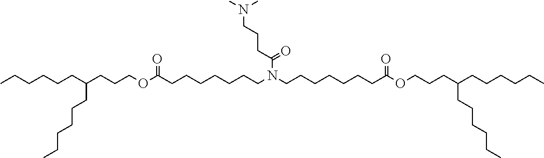 [SMILES: CCCCCCC(CCCCCC)CCCOC(=O)CCCCCCCN(CCCCCCCC(=O)OCCCC(CCCCCC)CCCCCC)C(=O)CCCN(C)C] | bis(4-hexyldecyl) 8,8′- ((4- (dimethylamino)butanoyl) azanediyl)dioctanoate |
| 2137 | 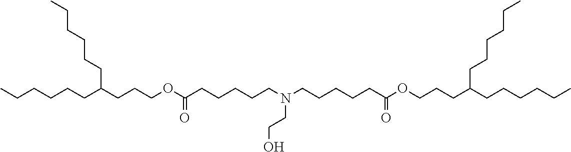 [SMILES: CCCCCCC(CCCCCC)CCCOC(=O)CCCCCN(CCO)CCCCCC(=O)OCCCC(CCCCCC)CCCCCC] | bis(4-hexyldecyl) 6,6′- ((2- hydroxyethyl)azanediyl) dihexanoate |
| 2131 | 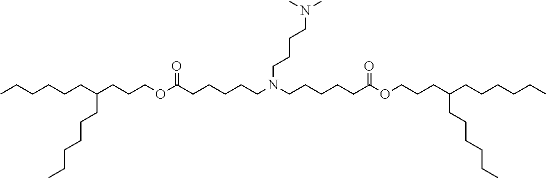 [SMILES: CCCCCCC(CCCCCC)CCCOC(=O)CCCCCN(CCCCCC(=O)OCCCC(CCCCCC)CCCCCC)CCCCN(C)C] | bis(4-hexyldecyl) 6,6′- ((4- (dimethylamino)butyl) azanediyl)dihexanoate |
| 2130 | 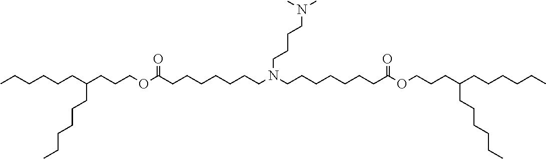 [SMILES: CCCCCCC(CCCCCC)CCCOC(=O)CCCCCCCN(CCCCCCCC(=O)OCCCC(CCCCCC)CCCCCC)CCCCN(C)C] | bis(4-hexyldecyl) 8,8′- ((4- (dimethylamino)butyl) azanediyl)dioctanoate |
| 2129 | 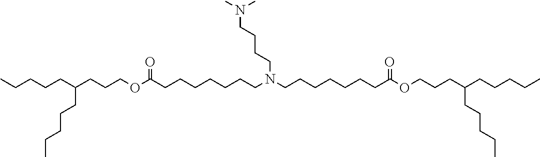 [SMILES: CCCCCC(CCCCC)CCCOC(=O)CCCCCCCN(CCCCCCCC(=O)OCCCC(CCCCC)CCCCC)CCCCN(C)C] | bis(4-pentylnonyl) 8,8′- ((4- (dimethylamino)butyl) azanediyl)dioctanoate |
| 2144 | 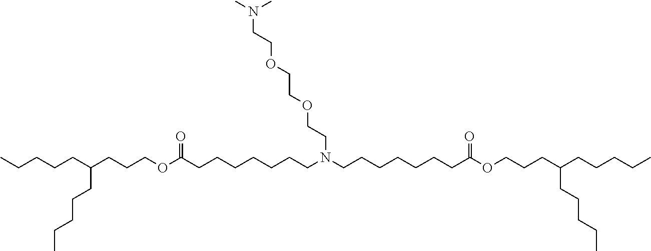 [SMILES: CCCCCC(CCCCC)CCCOC(=O)CCCCCCCN(CCCCCCCC(=O)OCCCC(CCCCC)CCCCC)CCOCCOCCN(C)C] | 4-pentylnonyl 2-methyl- 11-(8-oxo-8-((4- pentylnonyl)oxy)octyl)- 5,8-dioxa-2,11- diazanonadecan-19- oate |
| 2143 | 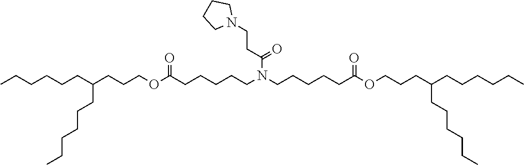 [SMILES: CCCCCCC(CCCCCC)CCCOC(=O)CCCCCN(CCCCCC(=O)OCCCC(CCCCCC)CCCCCC)C(=O)CCN1CCCC1] | bis(4-hexyldecyl) 6,6′- ((3-(pyrrolidin-1- yl)propanoyl)azanediyl) dihexanoate |
| 2134 | 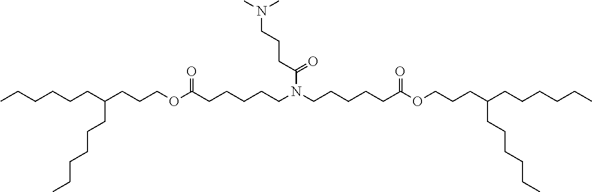 [SMILES: CCCCCCC(CCCCCC)CCCOC(=O)CCCCCN(CCCCCC(=O)OCCCC(CCCCCC)CCCCCC)C(=O)CCCN(C)C] | bis(4-hexyldecyl) 6,6′- ((4- (dimethylamino)butanoyl) azanediyl)dihexanoate |
| 2135 | 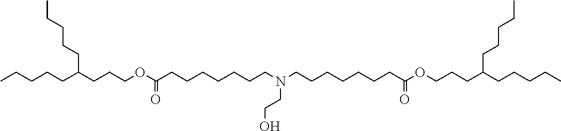 [SMILES: CCCCCC(CCCCC)CCCOC(=O)CCCCCCCN(CCO)CCCCCCCC(=O)OCCCC(CCCCC)CCCCC] | bis(4-pentylnonyl) 8,8′- ((2- hydroxyethyl)azanediyl) dioctanoate |
| 2136 | 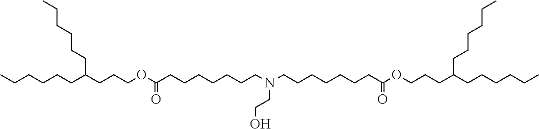 [SMILES: CCCCCCC(CCCCCC)CCCOC(=O)CCCCCCCN(CCO)CCCCCCCC(=O)OCCCC(CCCCCC)CCCCCC] | bis(4-hexyldecyl) 8,8′- ((2- hydroxyethyl)azanediyl) dioctanoate |
| 2141 | 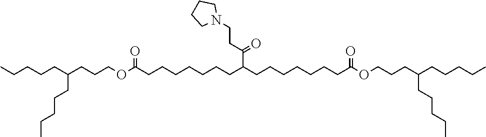 [SMILES: CCCCCC(CCCCC)CCCOC(=O)CCCCCCCC(CCCCCCCC(=O)OCCCC(CCCCC)CCCCC)C(=O)CCN1CCCC1] | bis(4-pentylnonyl) 8,8′- ((3-(pyrrolidin-1- yl)propanoyl)azanediyl) dioctanoate |
| 2132 | 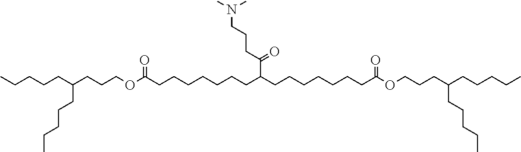 [SMILES: CCCCCC(CCCCC)CCCOC(=O)CCCCCCCC(CCCCCCCC(=O)OCCCC(CCCCC)CCCCC)C(=O)CCCN(C)C] | bis(4-pentylnonyl) 8,8′- ((4- (dimethylamino)butanoyl) azanediyl)dioctanoate |
| 2229 | 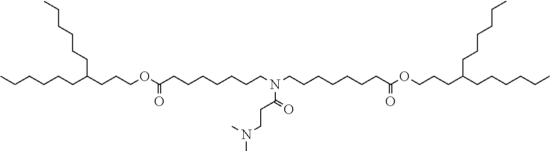 [SMILES: CCCCCCC(CCCCCC)CCCOC(=O)CCCCCCCN(CCCCCCCC(=O)OCCCC(CCCCCC)CCCCCC)C(=O)CCN(C)C] | bis(4-hexyldecyl) 8,8′- ((3-(dimethylamino) propanoyl) azanediyl)dioctanoate |
| 2228 | 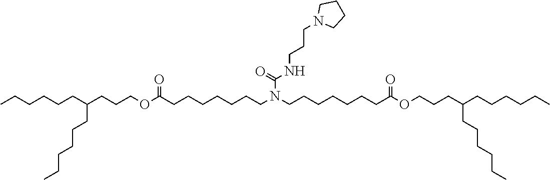 [SMILES: CCCCCCC(CCCCCC)CCCOC(=O)CCCCCCCN(CCCCCCCC(=O)OCCCC(CCCCCC)CCCCCC)C(=O)NCCCN1CCCC1] | bis(4-hexyldecyl) 8,8′- (((3-(pyrrolidin-1- yl)propyl)carbamoyl) azanediyl)dioctanoate |
| 2227 | 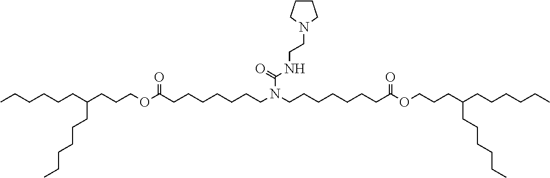 [SMILES: CCCCCCC(CCCCCC)CCCOC(=O)CCCCCCCN(CCCCCCCC(=O)OCCCC(CCCCCC)CCCCCC)C(=O)NCCN1CCCC1] | bis(4-hexyldecyl) 8,8′- (((2-(pyrrolidin-1- yl)ethyl)carbamoyl) azanediyl)dioctanoate |
| 2226 | 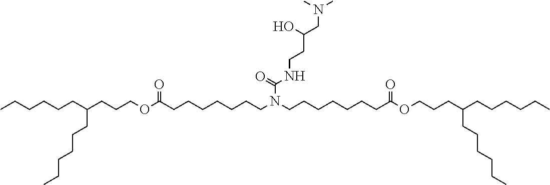 [SMILES: CCCCCCC(CCCCCC)CCCOC(=O)CCCCCCCN(CCCCCCCC(=O)OCCCC(CCCCCC)CCCCCC)C(=O)NCCC(O)CN(C)C] | bis(4-hexyldecyl) 8,8′- (((4-(dimethylamino)-3- hydroxybutyl)carbamoyl) azanediyl)dioctanoate |
| 2225 | 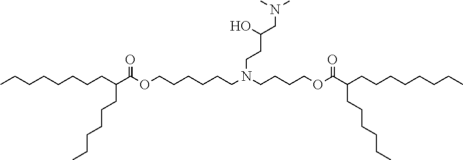 [SMILES: CCCCCCCCC(CCCCCC)C(=O)OCCCCCCN(CCCCOC(=O)C(CCCCCC)CCCCCCCC)CCC(O)CN(C)C] | ((4-(dimethylamino)-3- hydroxybutyl)azanediyl) bis(hexane-6,1-diyl) bis(2-hexyldecanoate) |
| 2216 | 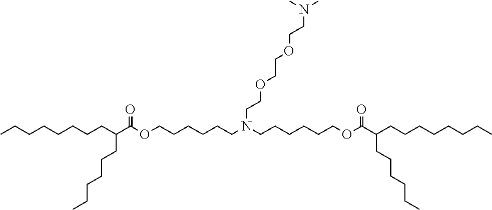 [SMILES: CCCCCCCCC(CCCCCC)C(=O)OCCCCCCN(CCCCCCOC(=O)C(CCCCCC)CCCCCCCC)CCOCCOCCN(C)C] | 11-(6-((2- hexyldecanoyl) oxy)hexyl)- 2-methyl-5,8-dioxa- 2,11-diazaheptadecan- 17-yl 2-hexyldecanoate |
| 2215 | 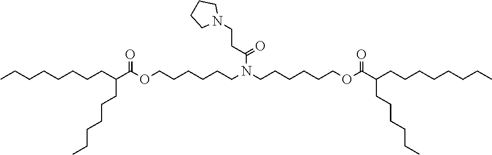 [SMILES: CCCCCCCCC(CCCCCC)C(=O)OCCCCCCN(CCCCCCOC(=O)C(CCCCCC)CCCCCCCC)C(=O)CCN1CCCC1] | ((3-(pyrrolidin-1- yl)propanoyl)azanediyl) bis(hexane-6,1-diyl) bis(2-hexyldecanoate) |
| | 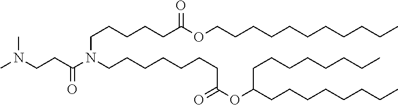 [SMILES: CCCCCCCCCCCOC(=O)CCCCCN(CCCCCCCC(=O)OC(CCCCCCCC)CCCCCCCC)C(=O)CCN(C)C] | heptadecan-9-yl 8-(3- (dimethylamino)-N-(6- oxo-6-(undecyloxy) hexyl)propanamido) octanoate |
| | 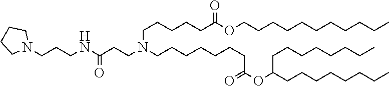 [SMILES: CCCCCCCCCCCOC(=O)CCCCCN(CCCCCCCC(=O)OC(CCCCCCCC)CCCCCCCC)CCC(=O)NCCCN1CCCC1] | heptadecan-9-yl 8-(1-(6- oxo-6- (undecyloxy)hexyl)-3-(3- (pyrrolidin-1- yl)propyl)ureido) octanoate |
| | 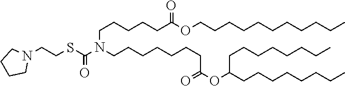 [SMILES: CCCCCCCCCCCOC(=O)CCCCCN(CCCCCCCC(=O)OC(CCCCCCCC)CCCCCCCC)C(=O)SCCN1CCCC1] | heptadecan-9-yl 8-((6- oxo-6- (undecyloxy)hexyl) (((2- (pyrrolidin-1- yl)ethyl)thio)carbonyl) amino)octanoate |
| | 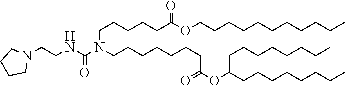 [SMILES: CCCCCCCCCCCOC(=O)CCCCCN(CCCCCCCC(=O)OC(CCCCCCCC)CCCCCCCC)C(=O)NCCN1CCCC1] | heptadecan-9-yl 8-(1-(6- oxo-6- (undecyloxy)hexyl)-3-(2- (pyrrolidin-1- yl)ethyl)ureido)octanoate |
| | 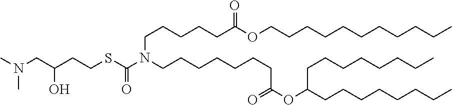 [SMILES: CCCCCCCCCCCOC(=O)CCCCCN(CCCCCCCC(=O)OC(CCCCCCCC)CCCCCCCC)C(=O)SCCC(O)CN(C)C] | heptadecan-9-yl 8-((((4- (dimethylamino)-3- hydroxybutyl)thio) carbonyl)(6-oxo-6- (undecyloxy)hexyl) amino)octanoate |
| | 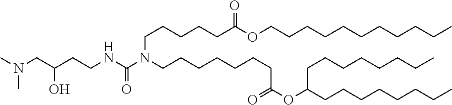 [SMILES: CCCCCCCCCCCOC(=O)CCCCCN(CCCCCCCC(=O)OC(CCCCCCCC)CCCCCCCC)C(=O)NCCC(O)CN(C)C] | heptadecan-9-yl 8-(3-(4- (dimethylamino)-3- hydroxybutyl)-1- (6-oxo-6- (undecyloxy)hexyl) ureido)octanoate |
| | 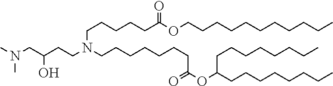 [SMILES: CCCCCCCCCCCOC(=O)CCCCCN(CCCCCCCC(=O)OC(CCCCCCCC)CCCCCCCC)CCC(O)CN(C)C] | heptadecan-9-yl 8-((4- (dimethylamino)-3- hydroxybutyl)(6-oxo-6- (undecyloxy)hexyl) amino)octanoate |
| | 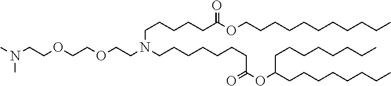 [SMILES: CCCCCCCCCCCOC(=O)CCCCCN(CCCCCCCC(=O)OC(CCCCCCCC)CCCCCCCC)CCOCCOCCN(C)C] | heptadecan-9-yl 2- methyl-11-(6-oxo-6- (undecyloxy)hexyl)-5,8- dioxa-2,11- diazanonadecan-19- oate |
| | 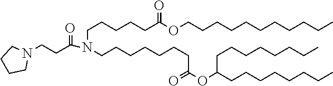 [SMILES: CCCCCCCCCCCOC(=O)CCCCCN(CCCCCCCC(=O)OC(CCCCCCCC)CCCCCCCC)C(=O)CCN1CCCC1] | heptadecan-9-yl 8-(N-(6- oxo-6- (undecyloxy)hexyl)-3- (pyrrolidin-1- yl)propanamido) octanoate |
| | 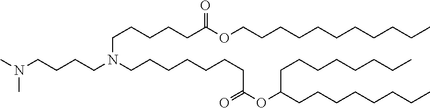 [SMILES: CCCCCCCCCCCOC(=O)CCCCCN(CCCCCCCC(=O)OC(CCCCCCCC)CCCCCCCC)CCCCN(C)C] | heptadecan-9-yl 8-((4- (dimethylamino)butyl) (6-oxo-6- (undecyloxy)hexyl) amino)octanoate |
| | 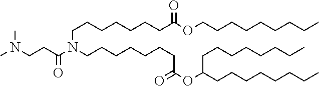 [SMILES: CCCCCCCCCOC(=O)CCCCCCCN(CCCCCCCC(=O)OC(CCCCCCCC)CCCCCCCC)C(=O)CCN(C)C] | heptadecan-9-yl 8-(3- (dimethylamino)-N-(8- (nonyloxy)-8- oxooctyl)propanamido) octanoate |
| | 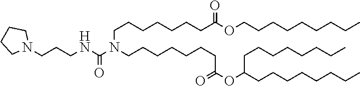 [SMILES: CCCCCCCCCOC(=O)CCCCCCCN(CCCCCCCC(=O)OC(CCCCCCCC)CCCCCCCC)C(=O)NCCCN1CCCC1] | heptadecan-9-yl 8-(1-(8- (nonyloxy)-8-oxooctyl)- 3-(3-(pyrrolidin-1- yl)propyl)ureido) octanoate |
| | 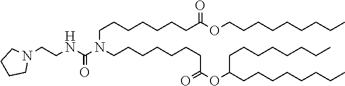 [SMILES: CCCCCCCCCOC(=O)CCCCCCCN(CCCCCCCC(=O)OC(CCCCCCCC)CCCCCCCC)C(=O)NCCN1CCCC1] | heptadecan-9-yl 8-(1-(8- (nonyloxy)-8-oxooctyl)- 3-(2-(pyrrolidin-1- yl)ethyl)ureido) octanoate |
| | 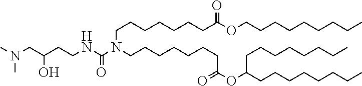 [SMILES: CCCCCCCCCOC(=O)CCCCCCCN(CCCCCCCC(=O)OC(CCCCCCCC)CCCCCCCC)C(=O)NCCC(O)CN(C)C] | heptadecan-9-yl 8-(3-(4- (dimethylamino)-3- hydroxybutyl)-1-(8- (nonyloxy)-8- oxooctyl)ureido) octanoate |
| | 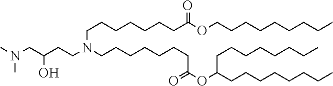 [SMILES: CCCCCCCCCOC(=O)CCCCCCCN(CCCCCCCC(=O)OC(CCCCCCCC)CCCCCCCC)CCC(O)CN(C)C] | heptadecan-9-yl 8-((4- (dimethylamino)-3- hydroxybutyl)(8- (nonyloxy)-8- oxooctyl)amino) octanoate |
| | 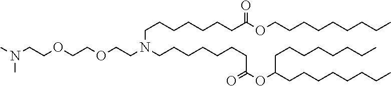 [SMILES: CCCCCCCCCOC(=O)CCCCCCCN(CCCCCCCC(=O)OC(CCCCCCCC)CCCCCCCC)CCOCCOCCN(C)C] | heptadecan-9-yl 2- methyl-11-(8-(nonyloxy)- 8-oxooctyl)-5,8-dioxa- 2, 11-diazanonadecan- 19-oate |
| | 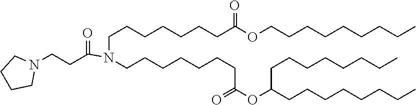 [SMILES: CCCCCCCCCOC(=O)CCCCCCCN(CCCCCCCC(=O)OC(CCCCCCCC)CCCCCCCC)C(=O)CCN1CCCC1] | heptadecan-9-yl 8-(N-(8- (nonyloxy)-8-oxooctyl)- 3-(pyrrolidin-1- yl)propanamido) octanoate |
| | 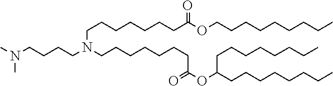 [SMILES: CCCCCCCCCOC(=O)CCCCCCCN(CCCCCCCC(=O)OC(CCCCCCCC)CCCCCCCC)CCCCN(C)C] | heptadecan-9-yl 8-((4- (dimethylamino)butyl)(8- (nonyloxy)-8- oxooctyl)amino) octanoate |
| | 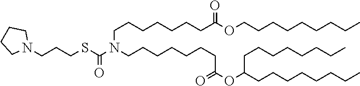 [SMILES: CCCCCCCCCOC(=O)CCCCCCCN(CCCCCCCC(=O)OC(CCCCCCCC)CCCCCCCC)C(=O)SCCCN1CCCC1] | heptadecan-9-yl 8-((8- (nonyloxy)-8- oxooctyl)(((3-(pyrrolidin- 1- yl)propyl)thio)carbonyl) amino)octanoate |
| | 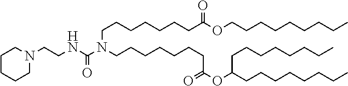 [SMILES: CCCCCCCCCOC(=O)CCCCCCCN(CCCCCCCC(=O)OC(CCCCCCCC)CCCCCCCC)C(=O)NCCN1CCCCC1] | heptadecan-9-yl 8-(1-(8- (nonyloxy)-8-oxooctyl)- 3-(2-(piperidin-1- yl)ethyl)ureido) octanoate |
| | 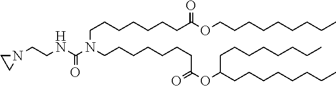 [SMILES: CCCCCCCCCOC(=O)CCCCCCCN(CCCCCCCC(=O)OC(CCCCCCCC)CCCCCCCC)C(=O)NCCN1CC1] | heptadecan-9-yl 8-(3-(2- (aziridin-1-yl)ethyl)-1-(8- (nonyloxy)-8- oxooctyl)ureido) octanoate |
| | 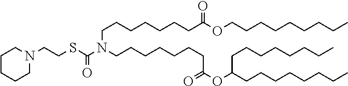 [SMILES: CCCCCCCCCOC(=O)CCCCCCCN(CCCCCCCC(=O)OC(CCCCCCCC)CCCCCCCC)C(=O)SCCN1CCCCC1] | heptadecan-9-yl 8-((8- (nonyloxy)-8- oxooctyl) (((2-(piperidin- 1- yl)ethyl)thio)carbonyl) amino)octanoate |
| | 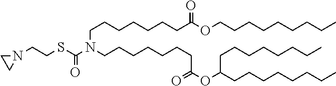 [SMILES: CCCCCCCCCOC(=O)CCCCCCCN(CCCCCCCC(=O)OC(CCCCCCCC)CCCCCCCC)C(=O)SCCN1CC1] | heptadecan-9-yl 8-((((2- (aziridin-1- yl)ethyl)thio)carbonyl)(8- (nonyloxy)-8- oxooctyl)amino) octanoate |
| | 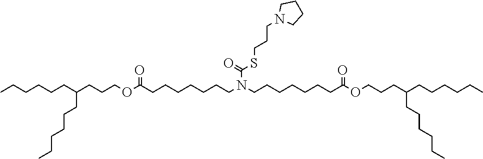 [SMILES: CCCCCCC(CCCCCC)CCCOC(=O)CCCCCCCN(CCCCCCCC(=O)OCCCC(CCCCCC)CCCCCC)C(=O)SCCCN1CCCC1] | bis(4-hexyldecyl) 8,8′- ((((3-(pyrrolidin-1- yl)propyl)thio)carbonyl) azanediyl)dioctanoate |
| | 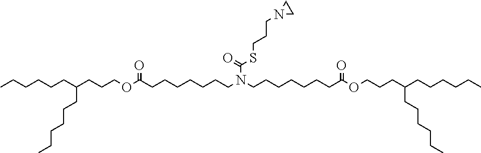 [SMILES: CCCCCCC(CCCCCC)CCCOC(=O)CCCCCCCN(CCCCCCCC(=O)OCCCC(CCCCCC)CCCCCC)C(=O)SCCCN1CC1] | bis(4-hexyldecyl) 8,8′- ((((3-(aziridin-1- yl)propyl)thio)carbonyl) azanediyl)dioctanoate |
| | 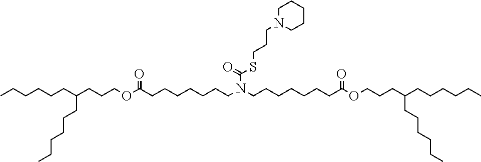 [SMILES: CCCCCCC(CCCCCC)CCCOC(=O)CCCCCCCN(CCCCCCCC(=O)OCCCC(CCCCCC)CCCCCC)C(=O)SCCCN1CCCCC1] | bis(4-hexyldecyl) 8,8′- ((((3-(piperidin-1- yl)propyl)thio)carbonyl) azanediyl)dioctanoate |
| | 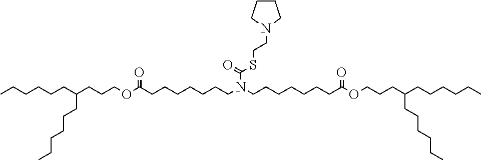 [SMILES: CCCCCCC(CCCCCC)CCCOC(=O)CCCCCCCN(CCCCCCCC(=O)OCCCC(CCCCCC)CCCCCC)C(=O)SCCN1CCCC1] | bis(4-hexyldecyl) 8,8′- ((((2-(pyrrolidin-1- yl)ethyl)thio)carbonyl) azanediyl)dioctanoate |
| | 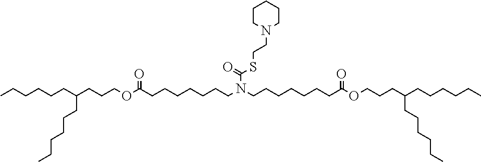 [SMILES: CCCCCCC(CCCCCC)CCCOC(=O)CCCCCCCN(CCCCCCCC(=O)OCCCC(CCCCCC)CCCCCC)C(=O)SCCN1CCCCC1] | bis(4-hexyldecyl) 8,8′- ((((2-(piperidin-1- yl)ethyl)thio)carbonyl) azanediyl)dioctanoate |
| | 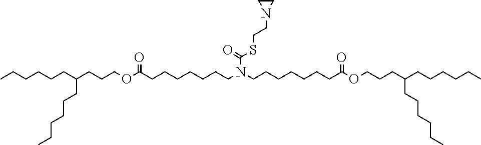 [SMILES: CCCCCCC(CCCCCC)CCCOC(=O)CCCCCCCN(CCCCCCCC(=O)OCCCC(CCCCCC)CCCCCC)C(=O)SCCN1CC1] | bis(4-hexyldecyl) 8,8′- ((((2-(aziridin-1- yl)ethyl)thio)carbonyl) azanediyl)dioctanoate |
| 2233 | 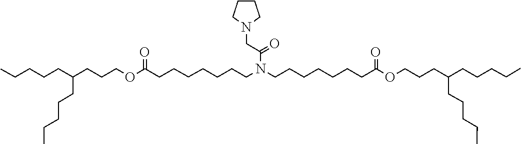 [SMILES: CCCCCC(CCCCC)CCCOC(=O)CCCCCCCN(CCCCCCCC(=O)OCCCC(CCCCC)CCCCC)C(=O)CN1CCCC1] | bis(4-pentylnonyl) 8,8′- ((2-(pyrrolidin-1- yl)acetyl)azanediyl) dioctanoate |
| 2234 | 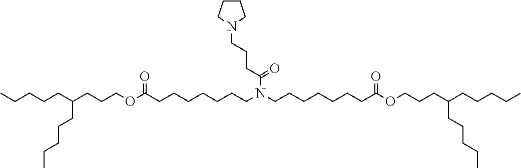 [SMILES: CCCCCC(CCCCC)CCCOC(=O)CCCCCCCN(CCCCCCCC(=O)OCCCC(CCCCC)CCCCC)C(=O)CCCN1CCCC1] | bis(4-pentylnonyl) 8,8′- ((4-(pyrrolidin-1- yl)butanoyl)azanediyl) dioctanoate |
| 2235 | 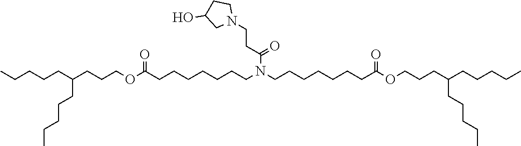 [SMILES: CCCCCC(CCCCC)CCCOC(=O)CCCCCCCN(CCCCCCCC(=O)OCCCC(CCCCC)CCCCC)C(=O)CCN1CCC(O)C1] | bis(4-pentylnonyl) 8,8′- ((3-(3-hydroxypyrrolidin- 1- yl)propanoyl)azanediyl) dioctanoate |
| 2236 | 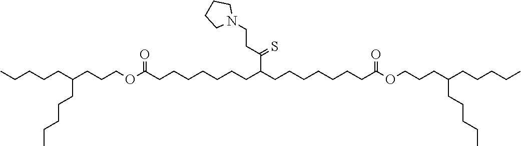 [SMILES: CCCCCC(CCCCC)CCCOC(=O)CCCCCCCC(CCCCCCCC(=O)OCCCC(CCCCC)CCCCC)C(=S)CCN1CCCC1] | bis(4-pentylnonyl) 8,8′- ((3-(pyrrolidin-1- yl)propanethioyl) azanediyl)dioctanoate |
| 2237 | 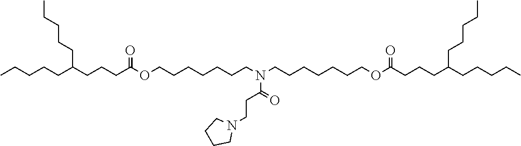 [SMILES: CCCCCC(CCCCC)CCCC(=O)OCCCCCCCN(CCCCCCCOC(=O)CCCC(CCCCC)CCCCC)C(=O)CCN1CCCC1] | ((3-(pyrrolidin-1- yl)propanoyl)azanediyl) bis(heptane-7,1-diyl) bis(5-pentyldecanoate) |
| 2238 | 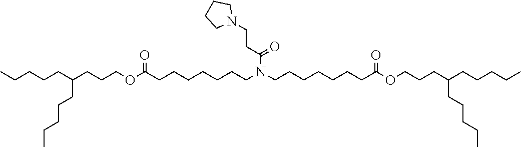 [SMILES: CCCCCC(CCCCC)CCCOC(=O)CCCCCCCN(CCCCCCCC(=O)OCCCC(CCCCC)CCCCC)C(=O)CCN1CCCC1] | ((3-(pyrrolidin-1- yl)propanoyl)azanediyl) bis(octane-8,1-diyl) bis(4- pentylnonanoate) |
| 2239 | 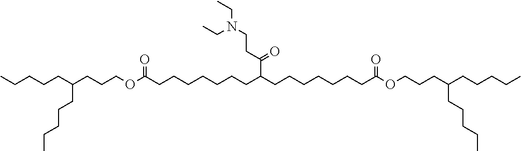 [SMILES: CCCCCC(CCCCC)CCCOC(=O)CCCCCCCC(CCCCCCCC(=O)OCCCC(CCCCC)CCCCC)C(=O)CCN(CC)CC] | bis(4-pentylnonyl) 8,8′- ((3- (diethylamino)propanoyl) azanediyl)dioctanoate |
| 2241 | 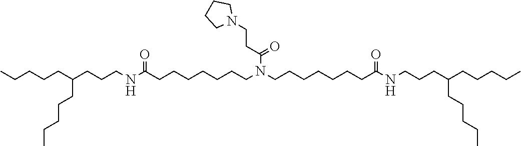 [SMILES: CCCCCC(CCCCC)CCCNC(=O)CCCCCCCN(CCCCCCCC(=O)NCCCC(CCCCC)CCCCC)C(=O)CCN1CCCC1] | 8-(N-(8-oxo-8-((4- pentylnonyl)amino)octyl)- 3-(pyrrolidin-1- yl)propanamido)-N-(4- pentylnonyl)octanamide |
| 2242 | 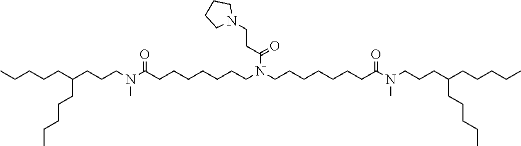 [SMILES: CCCCCC(CCCCC)CCCN(C)C(=O)CCCCCCCN(CCCCCCCC(=O)N(C)CCCC(CCCCC)CCCCC)C(=O)CCN1CCCC1] | N-methyl-8-(N-(8- (methyl(4- pentylnonyl)amino)-8- oxooctyl)-3-(pyrrolidin-1- yl)propanamido)-N-(4- pentylnonyl)octanamide |
| 2244 | 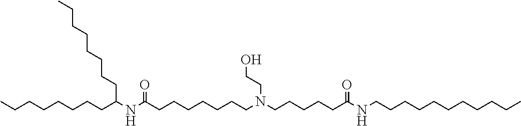 [SMILES: CCCCCCCCCCCNC(=O)CCCCCN(CCO)CCCCCCCC(=O)NC(CCCCCCCC)CCCCCCCC] | N-(heptadecan-9-yl)-8- ((2-hydroxyethyl)(6-oxo- 6- (undecylamino)hexyl) amino)octanamide |
| 2245 | 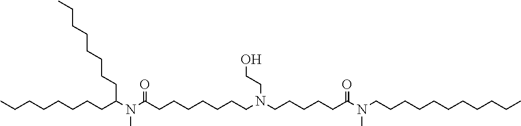 [SMILES: CCCCCCCCCCCN(C)C(=O)CCCCCN(CCO)CCCCCCCC(=O)N(C)C(CCCCCCCC)CCCCCCCC] | N-(heptadecan-9-yl)-8- ((2-hydroxyethyl)(6- (methyl(undecyl)amino)- 6-oxohexyl)amino)-N- methyloctanamide |
| 2249 | 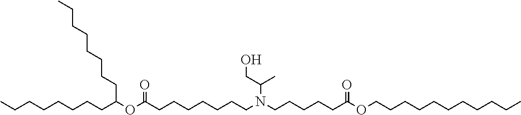 [SMILES: CCCCCCCCCCCOC(=O)CCCCCN(CCCCCCCC(=O)OC(CCCCCCCC)CCCCCCCC)C(C)CO] | heptadecan-9-yl 8-((1- hydroxypropan-2-yl)(6- oxo-6- (undecyloxy)hexyl) amino)octanoate |
| 2250 | 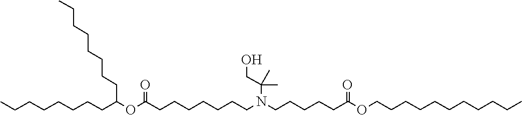 [SMILES: CCCCCCCCCCCOC(=O)CCCCCN(CCCCCCCC(=O)OC(CCCCCCCC)CCCCCCCC)C(C)(C)CO] | heptadecan-9-yl 8-((1- hydroxy-2- methylpropan-2-yl)(6- oxo-6- (undecyloxy)hexyl) amino)octanoate |
| 2276 | 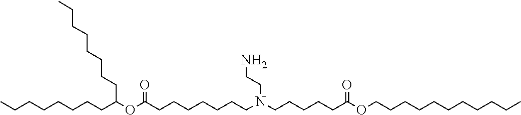 [SMILES: CCCCCCCCCCCOC(=O)CCCCCN(CCN)CCCCCCCC(=O)OC(CCCCCCCC)CCCCCCCC] | heptadecan-9-yl 8-((2- aminoethyl)(6-oxo-6- (undecyloxy)hexyl) amino)octanoate |
| 2277 | 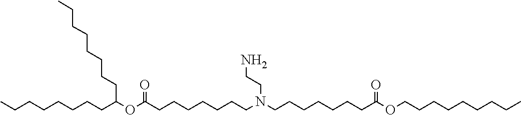 [SMILES: CCCCCCCCCOC(=O)CCCCCCCN(CCN)CCCCCCCC(=O)OC(CCCCCCCC)CCCCCCCC] | heptadecan-9-yl 8-((2- aminoethyl)(8- (nonyloxy)-8- oxooctyl)amino) octanoate |
| 2300 | 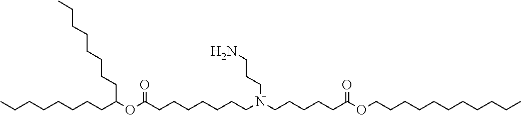 [SMILES: CCCCCCCCCCCOC(=O)CCCCCN(CCCN)CCCCCCCC(=O)OC(CCCCCCCC)CCCCCCCC] | heptadecan-9-yl 8-((3- aminopropyl)(6-oxo-6- (undecyloxy)hexyl) amino)octanoate |
| 2301 | 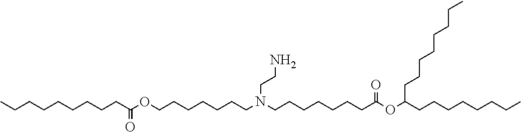 [SMILES: CCCCCCCCCC(=O)OCCCCCCCN(CCN)CCCCCCCC(=O)OC(CCCCCCCC)CCCCCCCC] | 7-((2-aminoethyl)(8- (heptadecan-9-yloxy)-8- oxooctyl)amino)heptyl decanoate |
| 2312 | 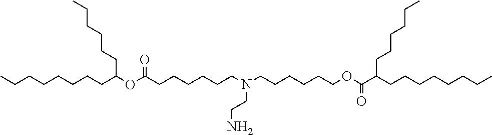 [SMILES: CCCCCCCCC(CCCCCC)OC(=O)CCCCCCN(CCN)CCCCCCOC(=O)C(CCCCCC)CCCCCCCC] | ((2- aminoethyl)azanediyl) bis(hexane-6,1-diyl) bis(2- hexyldecanoate) |
| 2313 | 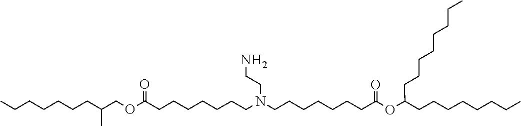 [SMILES: CCCCCCCCC(CCCCCCCC)OC(=O)CCCCCCCN(CCN)CCCCCCCC(=O)OCC(C)CCCCCCC] | heptadecan-9-yl 8-((2- aminoethyl) (8-((2- methylnonyl)oxy)-8- oxooctyl)amino) octanoate |
| 2314 | 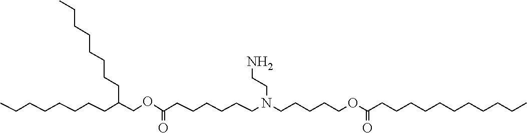 [SMILES: CCCCCCCCCCCC(=O)OCCCCCN(CCN)CCCCCCC(=O)OCC(CCCCCCCC)CCCCCCCC] | 5-((2-aminoethyl)(7-((2- octyldecanoyl)oxy)heptyl) amino)pentyl dodecanoate |
| | 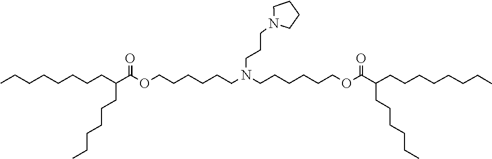 [SMILES: CCCCCCCCC(CCCCCC)C(=O)OCCCCCCN(CCCCCCOC(=O)C(CCCCCC)CCCCCCCC)CCCN1CCCC1] | ((3-(pyrrolidin-1- yl)propyl)azanediyl)bis (hexane-6,1-diyl) bis(2- hexyldecanoate) |
| | 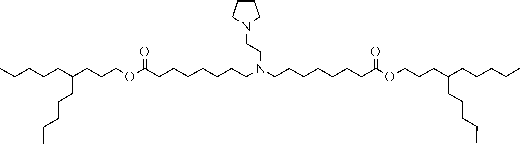 [SMILES: CCCCCC(CCCCC)CCCOC(=O)CCCCCCCN(CCCCCCCC(=O)OCCCC(CCCCC)CCCCC)CCN1CCCC1] | bis(4-pentylnonyl) 8,8′- ((2-(pyrrolidin-1- yl)ethyl)azanediyl) dioctanoate |
| | 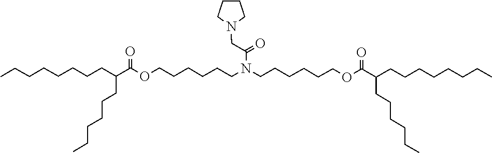 [SMILES: CCCCCCCCC(CCCCCC)C(=O)OCCCCCCN(CCCCCCOC(=O)C(CCCCCC)CCCCCCCC)C(=O)CN1CCCC1] | ((2-(pyrrolidin-1- yl)acetyl)azanediyl)bis (hexane-6,1-diyl) bis(2- hexyldecanoate) |
| | 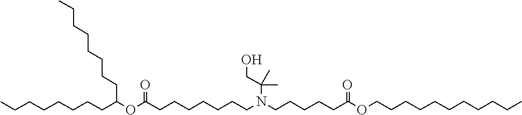 [SMILES: CCCCCCCCCCCOC(=O)CCCCCN(CCCCCCCC(=O)OC(CCCCCCCC)CCCCCCCC)C(C)(C)CO] | heptadecan-9-yl 8-((1- hydroxy-2- methylpropan-2-yl)(6- oxo-6- (undecyloxy)hexyl) amino)octanoate |
| | 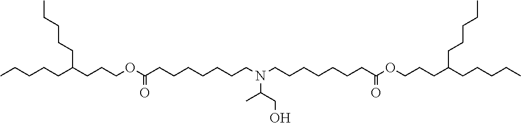 [SMILES: CCCCCC(CCCCC)CCCOC(=O)CCCCCCCN(CCCCCCCC(=O)OCCCC(CCCCC)CCCCC)C(C)CO] | bis(4-pentylnonyl) 8,8′- ((1-hydroxypropan-2- yl)azanediyl)dioctanoate |
| | 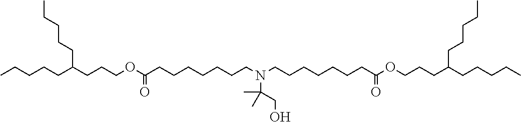 [SMILES: CCCCCC(CCCCC)CCCOC(=O)CCCCCCCN(CCCCCCCC(=O)OCCCC(CCCCC)CCCCC)C(C)(C)CO] | bis(4-pentylnonyl) 8,8′- ((1-hydroxy-2- methylpropan-2- yl)azanediyl)dioctanoate |
| | 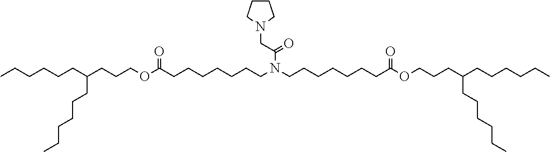 [SMILES: CCCCCCC(CCCCCC)CCCOC(=O)CCCCCCCN(CCCCCCCC(=O)OCCCC(CCCCCC)CCCCCC)C(=O)CN1CCCC1] | bis(4-hexyldecyl) 8,8′- ((2-(pyrrolidin-1- yl)acetyl)azanediyl) dioctanoate |
| | 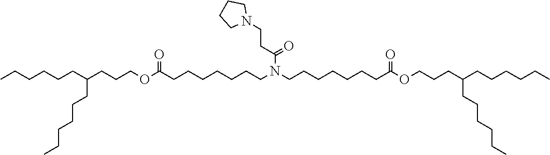 [SMILES: CCCCCCC(CCCCCC)CCCOC(=O)CCCCCCCN(CCCCCCCC(=O)OCCCC(CCCCCC)CCCCCC)C(=O)CCN1CCCC1] | bis(4-hexyldecyl) 8,8′- ((3-(pyrrolidin-1- yl)propanoyl)azanediyl) dioctanoate |
| | 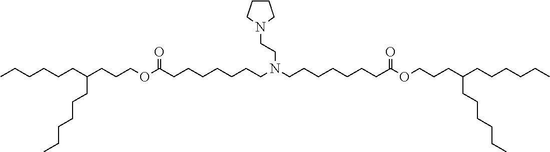 [SMILES: CCCCCCC(CCCCCC)CCCOC(=O)CCCCCCCN(CCCCCCCC(=O)OCCCC(CCCCCC)CCCCCC)CCN1CCCC1] | bis(4-hexyldecyl) 8,8′- ((2-(pyrrolidin-1- yl)ethyl)azanediyl) dioctanoate |
| | 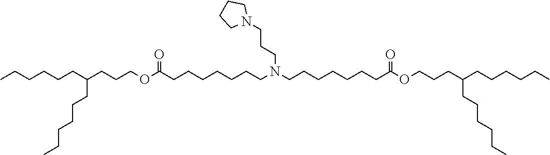 [SMILES: CCCCCCC(CCCCCC)CCCOC(=O)CCCCCCCN(CCCCCCCC(=O)OCCCC(CCCCCC)CCCCCC)CCCN1CCCC1] | bis(4-hexyldecyl) 8,8′- ((3-(pyrrolidin-1- yl)propyl)azanediyl) dioctanoate |
| | 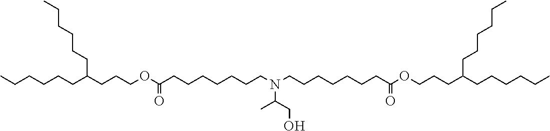 [SMILES: CCCCCCC(CCCCCC)CCCOC(=O)CCCCCCCN(CCCCCCCC(=O)OCCCC(CCCCCC)CCCCCC)C(C)CO] | bis(4-hexyldecyl) 8,8′- ((1-hydroxypropan-2- yl)azanediyl)dioctanoate |
| | 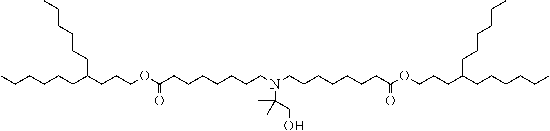 [SMILES: CCCCCCC(CCCCCC)CCCOC(=O)CCCCCCCN(CCCCCCCC(=O)OCCCC(CCCCCC)CCCCCC)C(C)(C)CO] | bis(4-hexyldecyl) 8,8′- ((1-hydroxy-2- methylpropan-2- yl)azanediyl)dioctanoate |
| | 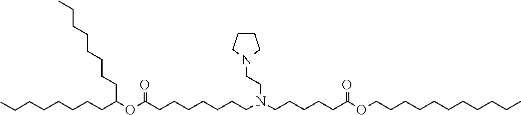 [SMILES: CCCCCCCCCCCOC(=O)CCCCCN(CCCCCCCC(=O)OC(CCCCCCCC)CCCCCCCC)CCN1CCCC1] | heptadecan-9-yl 8-((6- oxo-6- (undecyloxy)hexyl)(2- (pyrrolidin-1- yl)ethyl)amino)octanoate |
| | 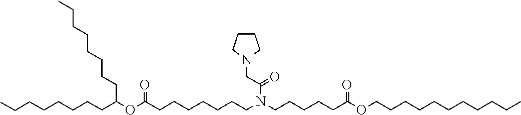 [SMILES: CCCCCCCCCCCOC(=O)CCCCCN(CCCCCCCC(=O)OC(CCCCCCCC)CCCCCCCC)C(=O)CN1CCCC1] | heptadecan-9-yl 8-(N-(6- oxo-6- (undecyloxy)hexyl)-2- (pyrrolidin-1- yl)acetamido)octanoate |
| | 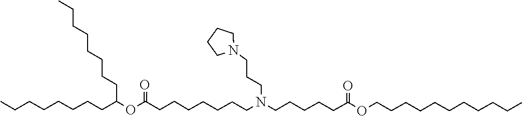 [SMILES: CCCCCCCCCCCOC(=O)CCCCCN(CCCCCCCC(=O)OC(CCCCCCCC)CCCCCCCC)CCCN1CCCC1] | heptadecan-9-yl 8-((6- oxo-6- (undecyloxy)hexyl)(3- (pyrrolidin-1- yl)propyl)amino) octanoate |
| | 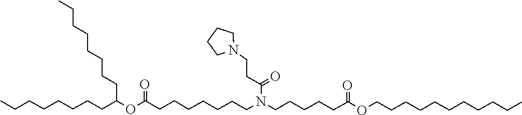 [SMILES: CCCCCCCCCCCOC(=O)CCCCCN(CCCCCCCC(=O)OC(CCCCCCCC)CCCCCCCC)C(=O)CCN1CCCC1] | heptadecan-9-yl 8-(N-(6- oxo-6- (undecyloxy)hexyl)-3- (pyrrolidin-1- yl)propanamido) octanoate |
| | 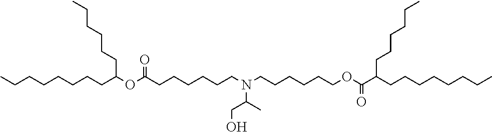 [SMILES: CCCCCCCCC(CCCCCC)OC(=O)CCCCCCN(CCCCCCOC(=O)C(CCCCCC)CCCCCCCC)C(C)CO] | ((1-hydroxypropan-2- yl)azanediyl)bis(hexane- 6,1-diyl) bis(2- hexyldecanoate) |
| | 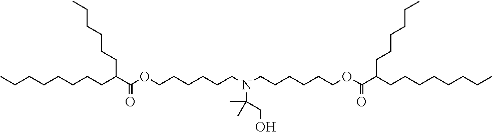 [SMILES: CCCCCCCCC(CCCCCC)C(=O)OCCCCCCN(CCCCCCOC(=O)C(CCCCCC)CCCCCCCC)C(C)(C)CO] | ((1-hydroxy-2- methylpropan-2- yl)azanediyl)bis(hexane- 6,1-diyl) bis(2- hexyldecanoate) |
| | 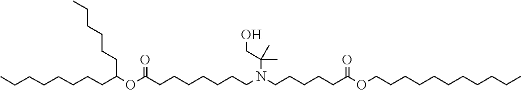 [SMILES: CCCCCCCCCCCOC(=O)CCCCCN(CCCCCCCC(=O)OC(CCCCCC)CCCCCCCC)C(C)(C)CO] | pentadecan-7-yl 8-((1- hydroxy-2- methylpropan-2-yl)(6- oxo-6- (undecyloxy)hexyl) amino)octanoate |
| | 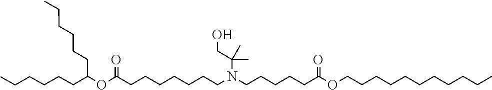 [SMILES: CCCCCCCCCCCOC(=O)CCCCCN(CCCCCCCC(=O)OC(CCCCCC)CCCCCC)C(C)(C)CO] | tridecan-7-yl 8-((1- hydroxy-2- methylpropan-2-yl)(6- oxo-6- (undecyloxy)hexyl) amino)octanoate |
| | 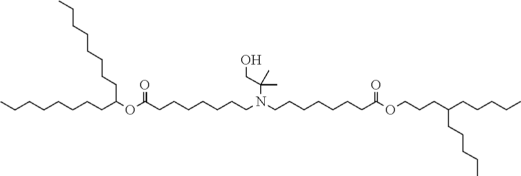 [SMILES: CCCCCCCCC(CCCCCCCC)OC(=O)CCCCCCCN(CCCCCCCC(=O)OCCCC(CCCCC)CCCCC)C(C)(C)CO] | heptadecan-9-yl 8-((1- hydroxy-2- methylpropan-2-yl)(8- oxo-8-((4- pentylnonyl)oxy)octyl) amino)octanoate |
| | 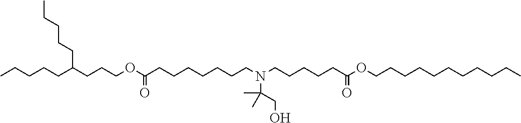 [SMILES: CCCCCCCCCCCOC(=O)CCCCCN(CCCCCCCC(=O)OCCCC(CCCCC)CCCCC)C(C)(C)CO] | 4-pentylnonyl 8-((1- hydroxy-2- methylpropan-2-yl)(6- oxo-6- (undecyloxy)hexyl) amino)octanoate |
| | 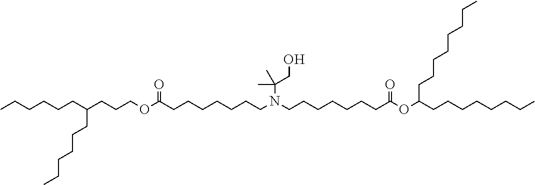 [SMILES: CCCCCCCCC(CCCCCCCC)OC(=O)CCCCCCCN(CCCCCCCC(=O)OCCCC(CCCCCC)CCCCCC)C(C)(C)CO] | heptadecan-9-yl 8-((8- ((4-hexyldecyl)oxy)-8- oxooctyl)(1-hydroxy-2- methylpropan-2- yl)amino)octanoate |
| | 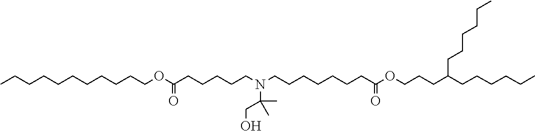 [SMILES: CCCCCCCCCCCOC(=O)CCCCCN(CCCCCCCC(=O)OCCCC(CCCCCC)CCCCCC)C(C)(C)CO] | 4-hexyldecyl 8-((1- hydroxy-2- methylpropan-2-yl)(6- oxo-6- (undecyloxy)hexyl) amino)octanoate |
| | 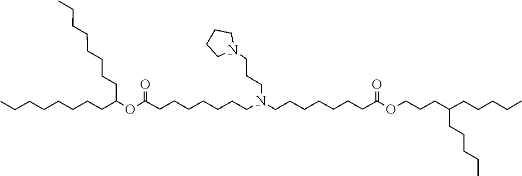 [SMILES: CCCCCCCCC(CCCCCCCC)OC(=O)CCCCCCCN(CCCCCCCC(=O)OCCCC(CCCCC)CCCCC)CCCN1CCCC1] | heptadecan-9-yl 8-((8- oxo-8-((4- pentylnonyl)oxy)octyl)(3- (pyrrolidin-1- yl)propyl)amino) octanoate |
| | 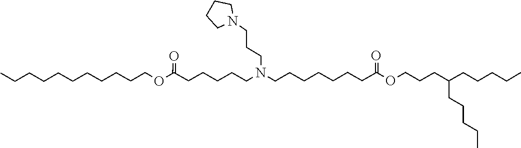 [SMILES: CCCCCCCCCCCOC(=O)CCCCCN(CCCCCCCC(=O)OCCCC(CCCCC)CCCCC)CCCN1CCCC1] | 4-pentylnonyl 8-((6-oxo- 6-(undecyloxy)hexyl)(3- (pyrrolidin-1- yl)propyl)amino) octanoate |
| | 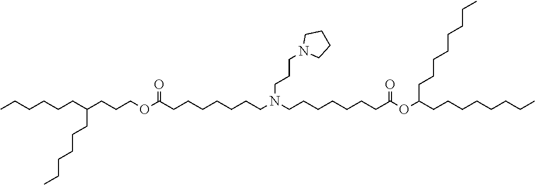 [SMILES: CCCCCCCCC(CCCCCCCC)OC(=O)CCCCCCCN(CCCCCCCC(=O)OCCCC(CCCCCC)CCCCCC)CCCN1CCCC1] | heptadecan-9-yl 8-((8- ((4-hexyldecyl)oxy)-8- oxooctyl)(3-(pyrrolidin-1- yl)propyl)amino) octanoate |
| | 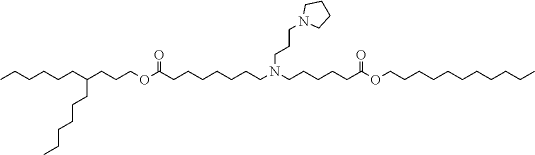 [SMILES: CCCCCCCCCCCOC(=O)CCCCCN(CCCCCCCC(=O)OCCCC(CCCCCC)CCCCCC)CCCN1CCCC1] | 4-hexyldecyl 8-((6-oxo- 6-(undecyloxy)hexyl)(3- (pyrrolidin-1- yl)propyl)amino) octanoate |
| | 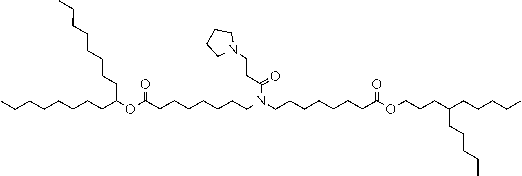 [SMILES: CCCCCCCCC(CCCCCCCC)OC(=O)CCCCCCCN(CCCCCCCC(=O)OCCCC(CCCCC)CCCCC)C(=O)CCN1CCCC1] | heptadecan-9-yl 8-(N-(8- oxo-8-((4- pentylnonyl)oxy)octyl)-3- (pyrrolidin-1- yl)propanamido) octanoate |
| | 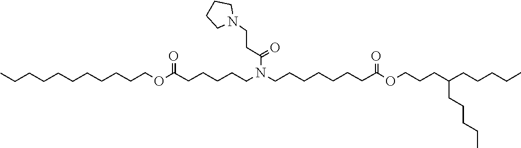 [SMILES: CCCCCCCCCCCOC(=O)CCCCCN(CCCCCCCC(=O)OCCCC(CCCCC)CCCCC)C(=O)CCN1CCCC1] | 4-pentylnonyl 8-(N-(6- oxo-6- (undecyloxy)hexyl)-3- (pyrrolidin-1- yl)propanamido) octanoate |
| | 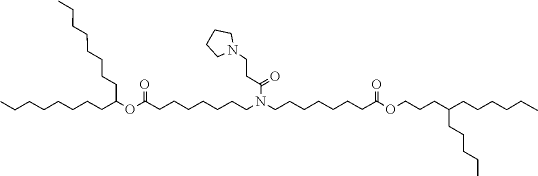 [SMILES: CCCCCCCCC(CCCCCCCC)OC(=O)CCCCCCCN(CCCCCCCC(=O)OCCCC(CCCCC)CCCCCC)C(=O)CCN1CCCC1] | heptadecan-9-yl 8-(N-(8- ((4-hexyldecyl)oxy)-8- oxooctyl)-3-(pyrrolidin-1- yl)propanamido) octanoate |
| | 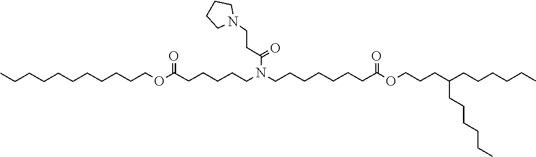 [SMILES: CCCCCCCCCCCOC(=O)CCCCCN(CCCCCCCC(=O)OCCCC(CCCCCC)CCCCCC)C(=O)CCN1CCCC1] | 4-hexyldecyl 8-(N-(6- oxo-6- (undecyloxy)hexyl)-3- (pyrrolidin-1- yl)propanamido) octanoate |
| | 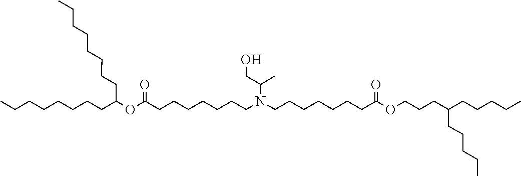 [SMILES: CCCCCCCCC(CCCCCCCC)OC(=O)CCCCCCCN(CCCCCCCC(=O)OCCCC(CCCCC)CCCCC)C(C)CO] | heptadecan-9-yl 8-((1- hydroxypropan-2-yl)(8- oxo-8-((4- pentylnonyl)oxy)octyl) amino)octanoate |
| | 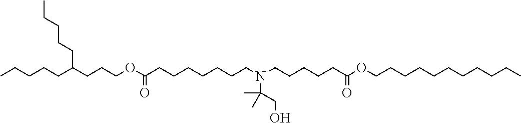 [SMILES: CCCCCCCCCCCOC(=O)CCCCCN(CCCCCCCC(=O)OCCCC(CCCCC)CCCCC)C(C)(C)CO] | 4-pentylnonyl 8-((1- hydroxypropan-2-yl)(6- oxo-6- (undecyloxy)hexyl) amino)octanoate |
| | 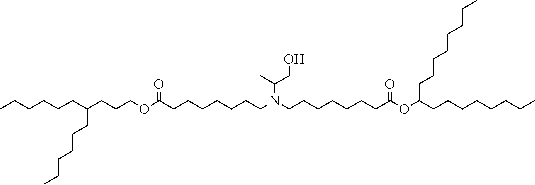 [SMILES: CCCCCCCCC(CCCCCCCC)OC(=O)CCCCCCCN(CCCCCCCC(=O)OCCCC(CCCCCC)CCCCCC)C(C)CO] | heptadecan-9-yl 8-((8- ((4-hexyldecyl)oxy)-8- oxooctyl)(1- hydroxypropan-2- yl)amino)octanoate |
| | 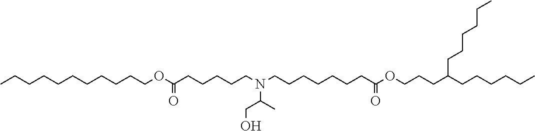 [SMILES: CCCCCCCCCCCOC(=O)CCCCCN(CCCCCCCC(=O)OCCCC(CCCCCC)CCCCCC)C(C)CO] | 4-hexyldecyl 8-((1- hydroxypropan-2-yl)(6- oxo-6- (undecyloxy)hexyl) amino)octanoate |
| | 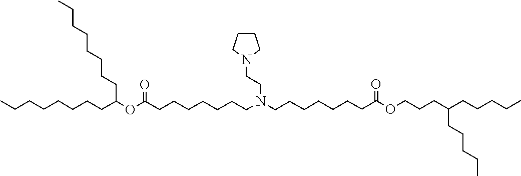 [SMILES: CCCCCCCCC(CCCCCCCC)OC(=O)CCCCCCCN(CCCCCCCC(=O)OCCCC(CCCCC)CCCCC)CCN1CCCC1] | heptadecan-9-yl 8-((8- oxo-8-((4- pentylnonyl)oxy)octyl)(2- (pyrrolidin-1- yl)ethyl)amino)octanoate |
| | 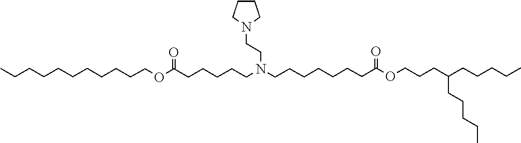 [SMILES: CCCCCCCCCCCOC(=O)CCCCCN(CCCCCCCC(=O)OCCCC(CCCCC)CCCCC)CCN1CCCC1] | 4-pentylnonyl 8-((6-oxo- 6-(undecyloxy)hexyl)(2- (pyrrolidin-1- yl)ethyl)amino)octanoate |
| | 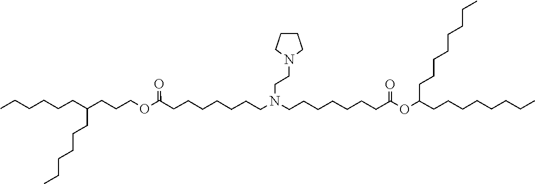 [SMILES: CCCCCCCCC(CCCCCCCC)OC(=O)CCCCCCCN(CCCCCCCC(=O)OCCCC(CCCCCC)CCCCCC)CCN1CCCC1] | heptadecan-9-yl 8-((8- ((4-hexyldecyl)oxy)-8- oxooctyl)(2-(pyrrolidin-1- yl)ethyl)amino)octanoate |
| | 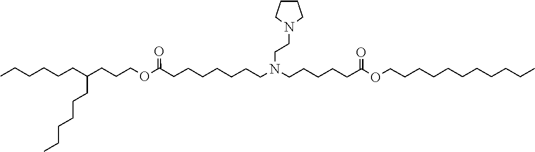 [SMILES: CCCCCCCCCCCOC(=O)CCCCCN(CCCCCCCC(=O)OCCCC(CCCCCC)CCCCCC)CCN1CCCC1] | 4-hexyldecyl 8-((6-oxo- 6-(undecyloxy)hexyl)(2- (pyrrolidin-1- yl)ethyl)amino)octanoate |
| | 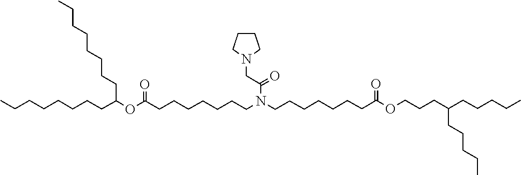 [SMILES: CCCCCCCCC(CCCCCCCC)OC(=O)CCCCCCCN(CCCCCCCC(=O)OCCCC(CCCCC)CCCCC)C(=O)CN1CCCC1] | heptadecan-9-yl 8-(N-(8- oxo-8-((4- pentylnonyl)oxy)octyl)-2- (pyrrolidin-1- yl)acetamido)octanoate |
| | 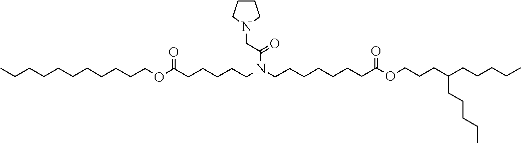 [SMILES: CCCCCCCCCCCOC(=O)CCCCCN(CCCCCCCC(=O)OCCCC(CCCCC)CCCCC)C(=O)CN1CCCC1] | 4-pentylnonyl 8-(N-(6- oxo-6- (undecyloxy)hexyl)-2- (pyrrolidin-1- yl)acetamido)octanoate |
| | 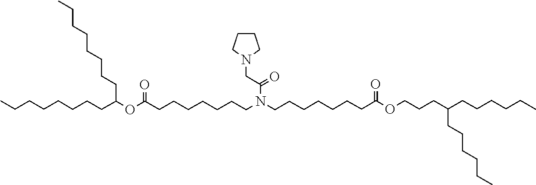 [SMILES: CCCCCCCCC(CCCCCCCC)OC(=O)CCCCCCCN(CCCCCCCC(=O)OCCCC(CCCCCC)CCCCCC)C(=O)CN1CCCC1] | heptadecan-9-yl 8-(N-(8- ((4-hexyldecyl)oxy)-8- oxooctyl)-2-(pyrrolidin-1- yl)acetamido)octanoate |
| | 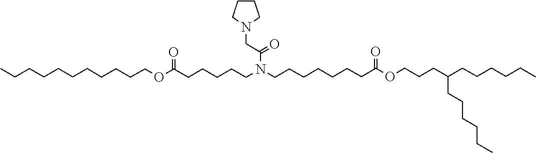 [SMILES: CCCCCCCCCCCOC(=O)CCCCCN(CCCCCCCC(=O)OCCCC(CCCCCC)CCCCCC)C(=O)CN1CCCC1] | 4-hexyldecyl 8-(N-(6- oxo-6- (undecyloxy)hexyl)-2- (pyrrolidin-1- yl)acetamido)octanoate |

### Ionizable Lipid Compounds iv)

In some embodiments, the ionizable lipid is a lipid comprising at least one head group and at least one tail group of formula (TI) or (TI′):

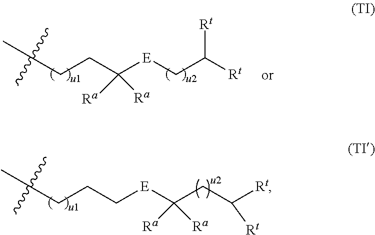

a pharmaceutically acceptable salt thereof, or a stereoisomer of any of the foregoing,
wherein:
E is each independently a biodegradable group;
R^{a} is each independently C₁-C₅ alkyl, C₂-C₅ alkenyl, or C₂-C₅ alkynyl;
u1 and u2 are each independently 0, 1, 2, 3, 4, 5, 6, or 7;
R^{t} is each independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
[symbol] represents the bond connecting the tail group to the head group; and
wherein the lipid has a pKa from about 4 to about 8.

In some embodiments, E is each independently —OC(O)—, —C(O)O—, —N(R⁷)C(O)—, —C(O)N(R⁷)—, —C(O—R₁₃)—O—, —C(O)O(CH₂)ᵣ—, —C(O)N(R⁷)(CH₂)ᵣ—, —C(O—R₁₃)—O—(CH₂)ᵣ—, or —S—S—, wherein each R⁷ is independently H, alkyl, alkenyl, cycloalkyl, hydroxyalkyl, or aminoalkyl; R₁₃ is branched or unbranched C₃-C₁₀ alkyl; and r is 1, 2, 3, 4, or 5. In some embodiments, E is each independently —OC(O)—, —C(O)O—, —N(R⁷)C(O)—, or —C(O)N(R⁷)—, wherein R⁷ is independently H, alkyl, alkenyl, cycloalkyl, hydroxyalkyl, or aminoalkyl.

In some embodiments, the lipid comprises at least one head group and at least one tail group of formula (TII):

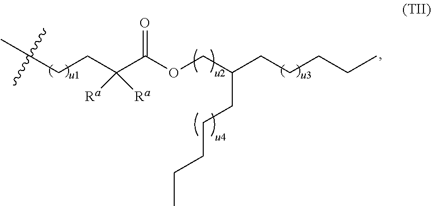

wherein u3 and u4 are each independently 0, 1, 2, 3, or 4. The definitions of other variables in (TII) are the same as those defined above in (TI).

In some embodiments, the lipid comprises at least one head group and at least one tail group of formula (TIII):

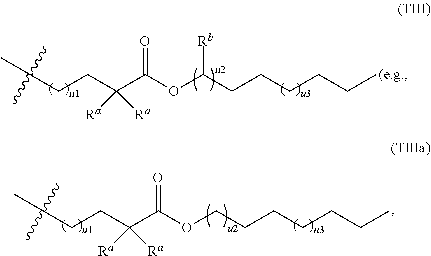

wherein u3 is 0, 1, 2, 3, 4, 5, 6, or 7; and R^{b} is in each occasion independently H or C₁-C₄ alkyl. The definitions of other variables in (TIII) are the same as those defined above in (TI).

In some embodiments, the lipid comprises at least one head group and at least one tail group of formula (TIV):

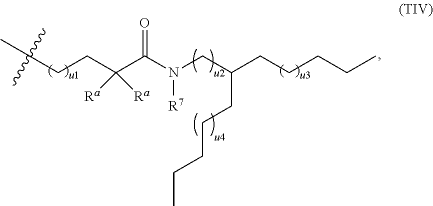

wherein u3 and u4 are each independently 0, 1, 2, 3, or 4. The definitions of other variables in (TIV) are the same as those defined above in (TI).

In some embodiments, the lipid comprises at least one head group and at least one tail group of formula (TV)

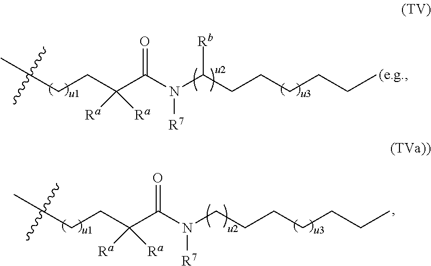

wherein u3 is 0, 1, 2, 3, 4, 5, 6, or 7; R⁷ is each independently H or methyl; and R^{b} is in each occasion independently H or C₁-C₄ alkyl. The definitions of other variables in (TV) are the same as those defined above in (TI).

In some embodiments, the lipid comprises at least one head group and at least one tail group of formula (TII′)

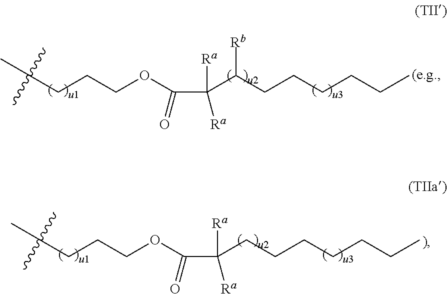

wherein u3 is 0, 1, 2, 3, 4, 5, 6, or 7; and R^{b} is in each occasion independently H or C₁-C₄ alkyl. The definitions of other variables in (TII′) are the same as those defined above in (TI′).

In some embodiments, the lipid comprises at least one head group and at least one tail group of formula (TII′)

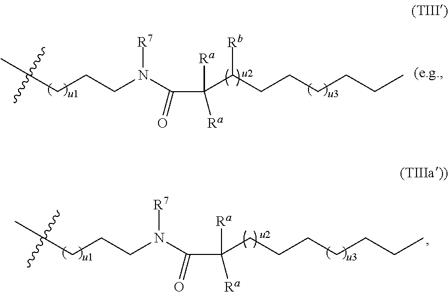

wherein u3 is 0, 1, 2, 3, 4, 5, 6, or 7; R⁷ is each independently H or methyl; and R^{b} is in each occasion independently H or C₁-C₄ alkyl. The definitions of other variables in (TIII′) are the same as those defined above in (TI′).

In some embodiments, the lipid comprises at least one tail group of the formulas (TII), (TIII), (TIV), (TV), (TII′), and (TIII′), wherein
R⁷ is each independently H or methyl;
R^{b} is in each occasion independently H or C₁-C₄ alkyl;
u1 and u2 are each independently 0, 1, 2, 3, 4, 5, 6, or 7;
u3 and u4 are each independently 0, 1, 2, 3, 4, 5, 6, or 7; and
wherein the lipid has a pKa from about 4 to about 8.

In some embodiments, the lipid comprises two, three, four, or more tail groups that have a formula of (T), (TI), (TII), (TIII), (TIV), (TV), (TII′), and/or (TIII′), and each tail group may be the same or different.

In some embodiments, in any of the above formulas (T), (TI), (TII), and (TIII), (TIV), (TV), (TI′), (TII′), and/or (TIII′), R^{a} is each independently C₁-C₅ branched or unbranched alkyl, C₂-C₅ branched or unbranched alkenyl, or C₂-C₅ branched or unbranched alkynyl. In some embodiments, R^{a} is each independently C₁-C₃ branched or unbranched alkyl. In one embodiment, each R^{a} is methyl.

In some embodiments, in any of the above formulas (T), (TI), (TII), and (TIII), (TIV), (TV), (TI′), (TII′), and/or (TIII′), u1 is 3, 4, or 5. In some embodiments, in any of the above formulas (T), (TI), (TII), and (TIII), (TIV), (TV), (TI′), (TII′), and/or (TIII′), u2 is 0, 1, 2, or 3. In some embodiments, in any of the above formulas (T), (TI), (TII), and (TIII), (TIV), (TV), (TI′), (TII′), and/or (TIII′), u3 and u4 are each independently 1-7, for instance, u3 and u4 are each independently 1, 2, 3, or 4.

In some embodiments, the lipid comprises at least one tail of formula (TIII), wherein each R^{a} is methyl; R^{b} is in each occasion independently H, ethyl, or butyl; u1 is 3-5, u2 is 0-3, and u3 is 1-7 (e.g., 1-4). In some embodiments, the lipid comprises at least one two tails of formula (TIII), wherein the two tails of formula (TIII) are the same or different. In some embodiments, the lipid comprises at least three tails of formula (TIII), wherein each tail may be the same or different. In some embodiments, the lipid has four tails of formula (TIII), wherein each tail may be the same or different. In some embodiments, in each tail of formula (TIII), each R^{a} is methyl, and u1 is 3, u2 is 2, and u3 is 4.

In some embodiments, the lipid comprises at least one tail of formula (TII), wherein each R^{a} is methyl, u1 is 3-5, u2 is 0-3, u3 is 1-4, and u4 is 1-4. In some embodiments, the lipid has at least two tails of formula (TII), wherein the two tails of formula (TII) are the same. In some embodiments, the lipid has at least two tails of formula (TII), wherein the two tails of formula (TII) are or different. In some embodiments, the lipid comprises at least three tails of formula (TII), wherein each tail may be the same or different. In some embodiments, the lipid has four tails of formula (TII), wherein each tail may be the same or different.

In some embodiments, the lipid comprises at least one tail of formula (TIV), wherein each R^{a} is methyl, u1 is 3-5, u2 is 0-3, u3 is 1-4, and u4 is 1-4. In some embodiments, the lipid comprises at least two tails of formula (TIV), wherein each tail may be the same or different. In some embodiments, the lipid comprises at least three tails of formula (TIV), wherein each tail may be the same or different. In some embodiments, the lipid comprises at least four tails of formula (TIV), wherein each tail may be the same or different.

In some embodiments, the lipid comprises at least two tails of formula (TV), wherein each tail may be the same or different. In some embodiments, the lipid comprises at least three tails of formula (TV), wherein each tail may be the same or different. In some embodiments, the lipid comprises at least four tails of formula (TV), wherein each tail may be the same or different.

In some embodiments, the lipid has at least two tails of formula (TII′), wherein each tail may be the same or different. In some embodiments, the lipid has at least three tails of formula (TII′), wherein each tail may be the same or different. In some embodiments, the lipid has at least four tails of formula (TII′), wherein each tail may be the same or different.

In some embodiments, the lipid has at least two tails of formula (TIII′), wherein each tail may be the same or different. In some embodiments, the lipid has at least three tails of formula (TIII′), wherein each tail may be the same or different. In some embodiments, the lipid has at least four tails of formula (TIII′), wherein each tail may be the same or different. In some embodiments, the lipid has at least one tail of formula (TII) and/or at least one tail of formula (TIII); the lipid further comprises at least one tail that does not have a formula (T), (TI), (TII), (TIII), (TIV), (TV), (TII′), and/or (TIII′). That is to say, the lipid further comprises at least one tail that does not contain a gem-di functional groups bonded to the same carbon next to E (e.g., —C(O)O—).

In some embodiments, the lipid further comprises at least one tail that does not have a formula (T), (TI), (TII), (TIII), (TIV), (TV), (TI′), (TII′), and/or (TIII′). That is to say, the lipid further comprises at least one tail that does not contain a gem-di functional groups bonded to the same carbon next to E.

In some embodiments, the lipid further comprises at least one tail that does not have a formula (T), (TI), (TII), (TIII), (TIV), (TV), (TI′), (TII′), and/or (TIII′). That is to say, the lipid further comprises at least one tail that does not contain a gem-di functional groups bonded to the same carbon next to E.

In some embodiments, the lipid further comprises at least one tail of formula (TNG-I):

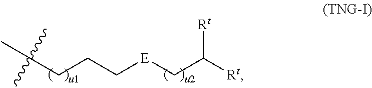

wherein
E is each independently a biodegradable group as described herein (e.g., —OC(O)—, —C(O)O—, —N(R⁷)C(O)—, —S—S—, or —C(O)N(R₇)—);
u1 and u2 are each independently 0, 1, 2, 3, 4, 5, 6, or 7; and
R⁷ is independently H, alkyl, alkenyl, cycloalkyl, hydroxyalkyl, or aminoalkyl.

In some embodiments, the at least one tail of formula (TNG-I) can be represented by

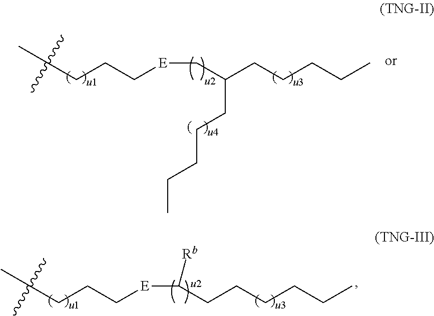

wherein
u3 and u4 are each independently 0, 1, 2, 3, 4, 5, 6, or 7; and
R^{b} is in each occasion independently H or C₁-C₄ alkyl.

All the embodiments above regarding the definitions of E, R^{b}, R^{t}, u1, u2, u3 and u4, as described above relating to the tail group containing a gem-di functional group bonded to the same carbon next to E, having a formula (T), (TI), (TII), (TIII), (TIV), (TV), (TII′), or (TIII′), are also applicable to the tail group that does not contain a gem-di functional groups bonded to the same carbon next to E, having a formula (TNG-I), (TNG-II), or (TNG-III).

In some embodiments, the lipid further comprises at least two tails that do not have a formula (T), (TI), (TII), (TIII), (TIV), (TV), (TI′), (TII′), and/or (TIII′). In some embodiments, the lipid comprises two tail groups of formula (TNG-II) or (TNG-III), and wherein each tail group may be the same or different,

In some embodiments, the lipid further comprises at least three tails that do not have a formula (T), (TI), (TII), (TIII), (TIV), (TV), (TI′), (TII′), and/or (TIII′). In some embodiments, the lipid comprises three tail groups of formula (TNG-II) or (TNG-III), and wherein each tail group may be the same or different,
In some embodiments, the head group of the lipid has a structure of formula (HA-I):

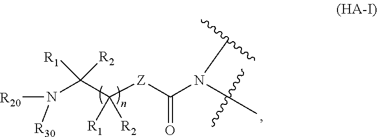

wherein:
R₂₀ and R₃₀ are each independently H, C₁-C₅ branched or unbranched alkyl, or C₂-C₅ branched or unbranched alkenyl, optionally interrupted with one or more heteroatoms or substituted with OH, SH, halogen, or cycloalkyl groups; or
R₂₀ and R₃₀, together with the adjacent N atom, form a 3 to 7 membered heterocyclic or heteroaromatic ring containing one or more heteroatoms, optionally substituted with one or more OH, SH, halogen, alkyl, or cycloalkyl groups;
each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ together form a cyclic ring;
each of R₁₀ and R₁₁ is independently H, C₁-C₃ branched or unbranched alkyl, C₂-C₃ branched or unbranched alkenyl; or R₁₀ and R₁₁ together form a heterocyclic ring;
n is 0, 1, 2, 3 or 4;
Z is absent, O, S, or NR₁₂, wherein R₁₂ is H or C₁-C₇ branched or unbranched alkyl; provided that when Z is not absent, the adjacent R₁ and R₂ cannot be OH, NR₁₀R₁₁, SH.

In some embodiments, R₂₀ and R₃₀ together with the adjacent N atom form a 3 to 7 membered heterocyclic or heteroaromatic ring containing one or more heteroatoms, optionally substituted with one or more OH, SH, halogen, alkyl, or cycloalkyl groups.

In some embodiments, the head group of the ionizable lipid has a structure of formula (HA-IA):

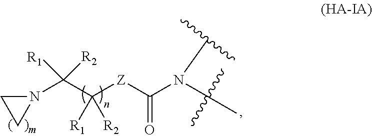

wherein:
each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ are taken together to form a cyclic ring;
each of R₁₀ and R₁₁ is independently H, C₁-C₃ branched or unbranched alkyl, C₂-C₃ branched or unbranched alkenyl; or R₁₀ and R₁₁ are taken together to form a heterocyclic ring;
m is 1, 2, 3, 4, 5, 6, 7 or 8;
n is 0, 1, 2, 3 or 4;
Z is absent, O, S, or NR₁₂, wherein R₁₂ is H or C₁-C₇ branched or unbranched alkyl; provided that when Z is not absent, the adjacent R₁ and R₂ cannot be OH, NR₁₀R₁₁, or SH; and
[symbol] represents the bond connecting the head group to the tail group.

In some embodiments, the head group of the ionizable lipid has a structure of formula (HA-III):

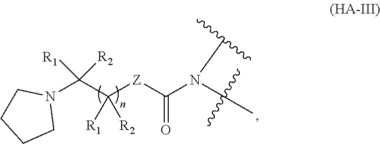

wherein Z is absent, O, S, or NR₁₂; and R₁₂ is H or C₁-C₇ branched or unbranched alkyl. The definitions of other variables in (HA-III) are the same as those defined above in (HA-IA).

In some embodiments, the head group has a structure of:

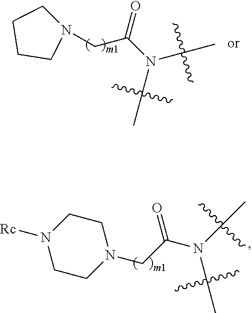

wherein:
Rc is H or alkyl, optionally substituted with OH; and
m1 is 1, 2, or 3.

In some embodiments, the head group of the ionizable lipid has a structure of formula (HA-V):

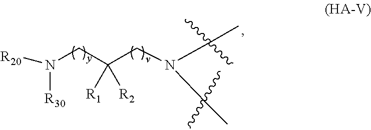

wherein:
R₁ is H, C₁-C₃ alkyl, OH, halogen, SH, or NR₁₀R₁₁;
R₂ is OH, halogen, SH, or NR₁₀R₁₁; or R₁ and R₂ can be taken together to form a cyclic ring;
R₁₀ and R₁₁ are each independently H or C₁-C₃ alkyl; or R₁₀ and R₁₁ can be taken together to form a heterocyclic ring;
R₂₀ and R₃₀ are each independently H, C₁-C₅ branched or unbranched alkyl, C₂-C₅ branched or unbranched alkenyl; or R₂₀ and R₃₀ can be taken together to form a cyclic ring; and
each of v and y is independently 1, 2, 3, or 4.

In some embodiments, the head group of the ionizable lipid has a structure of formula (HA-VI):

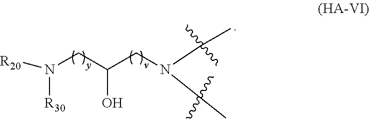

The definitions of all variables in (HA-VI) are the same as those defined above in (HA-V).

In some embodiments, in any of the above formulas (HA-V) or (HA-VI), each of R₂₀ and R₃₀ are independently C₁-C₃ alkyl. In one embodiment, each of R₂₀ and R₃₀ are independently methyl.

In some embodiments, the head group of the ionizable lipid has a structure of formula (HB—I):

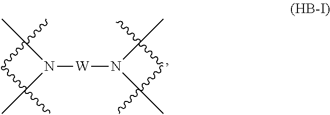

wherein W is

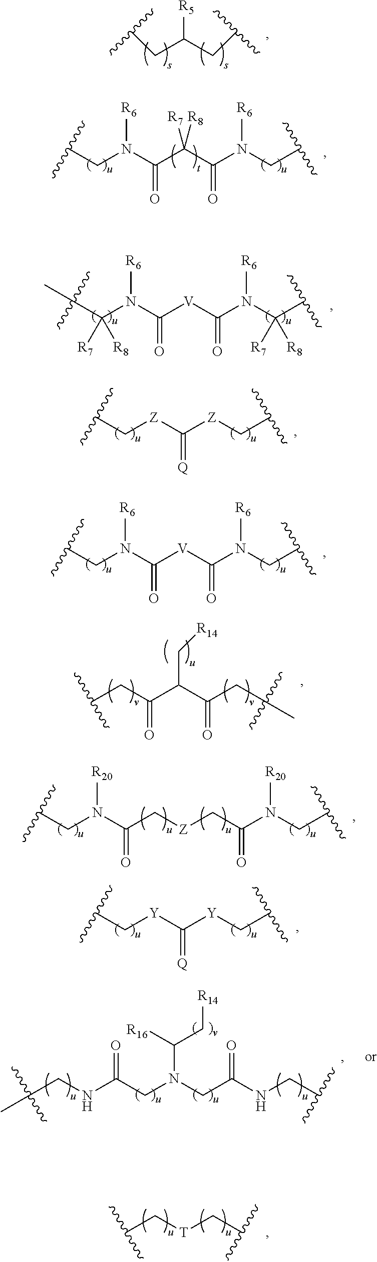

wherein
R₅ is OH, SH, (CH₂)ₛOH, or NR₁₀R₁₁;
each R₆ is independently H, C₁-C₃ branched or unbranched alkyl, C₂-C₃ branched or unbranched alkenyl, or cycloalkyl;
each R₇ and R₈ are independently H, C₁-C₃ branched or unbranched alkyl, C₂-C₃ branched or unbranched alkenyl, halogen, (CH₂)ᵥOH, (CH₂)ᵥSH, (CH₂)ₛN(CH₃)₂, or NR₁₀R₁₁, wherein each R₁₀ and R₁₁ is independently H or C₁-C₃ alkyl, or R₁₀ and R₁₁ are taken together to form a heterocyclic ring; or
R₇ and R₈ are taken together to form a ring;
each R₂₀ is independently H, or C₁-C₃ branched or unbranched alkyl;
R₁₄ is a heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H, C₁-C₃ alkyl, C₃-C₇ cycloalkyl, C₃-C₇ cycloalkenyl, optionally substituted with one or more NH and/or oxo groups, or R₁₀ and R₁₁ are taken together to form a heterocyclic ring;
R₁₆ is H, ═O, ═S, or CN;
each of s, u, and t is independently 1, 2, 3, 4, or 5;
each v is independently 0, 1, 2, 3, 4, or 5;
each Y is a divalent heterocyclic;
each Z is independently absent, O, S, or NR₁₂, wherein R₁₂ is H, C₁-C₇ branched or unbranched alkyl, or C₂-C₇ branched or unbranched alkenyl;
Q is O, S, CH₂, or NR₁₃, wherein each R₁₃ is H, or C₁-C₅ alkyl;
V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups; and
T is —NHC(O)O—, —OC(O)NH—, or a divalent heterocyclic.

In some embodiments, in formula (HB—I), W is

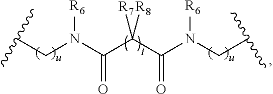

wherein:
each R₆, R₇, and R₈ are independently H or methyl; and
each of u and t is independently 1, 2, or 3.

In some embodiments, in formula (HB—I), W is

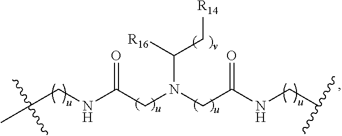

wherein:
R₁₆ is H or ═O;
R₁₄ is a nitrogen-containing 5- or 6-membered heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H or C₁-C₃ alkyl; and
each of u and v is independently 1, 2, or 3.

In some embodiments, in formula (HB—I), W is

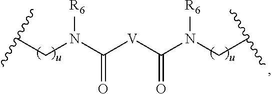

wherein:
each R₆ is independently H or methyl;
each u is independently 1, 2, or 3; and
V is C₂-C₆ alkylene or C₂-C₆ alkenylene.

In some embodiments, in formula (HB—I), W is

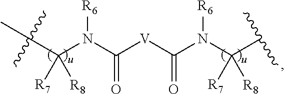

wherein:
each R₆ is independently H or methyl;
each R₇ is independently H;
each R₈ is methyl;
each u is independently 1, 2, or 3; and
V is C₂-C₆ alkylene or C₂-C₆ alkenylene.

In some embodiments, in formula (HB—I), W is

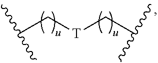

wherein:
each u is independently 1, 2, or 3; and
T is a divalent nitrogen-containing 5- or 6-membered heterocyclic.

In some embodiments, in formula (HB—I), W is

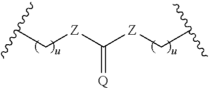

wherein:
each u is independently 1, 2, or 3;
Q is O;
each Z is independently NR₁₂; and
R₁₂ is H or C₁-C₃ alkyl.

In some embodiments, the head group has the structure of:

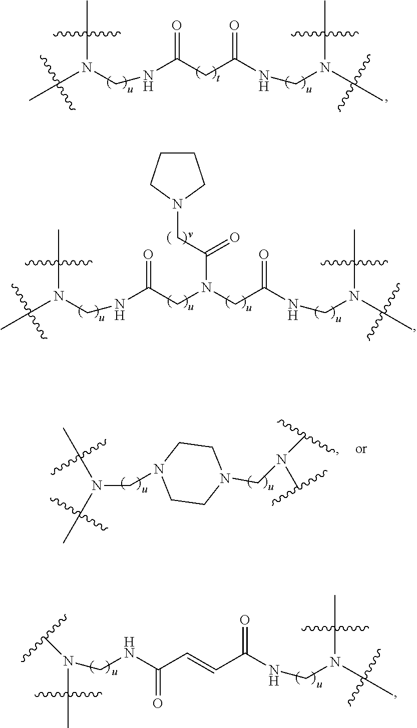

wherein each of u and t is independently 1 or 2.

In some embodiments, the head group of the ionizable lipid has a structure of formula (HC—I):

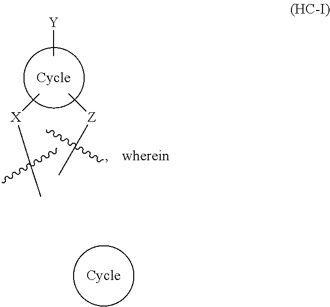

is a cyclic or heterocyclic moiety;
Y is alkyl, hydroxy, hydroxyalkyl,

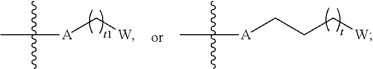

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, or —S—S—;
each of X and Z is independently absent, —O—, —C(O)—, —N(R⁷)—, —O-alkylene-, -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, alkoxy, hydroxyalkyl, alkylamino, alkylaminoalkyl, or aminoalkyl;
t is 0, 1, 2, or 3;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl.

In some embodiments, the head group has a structure of formula

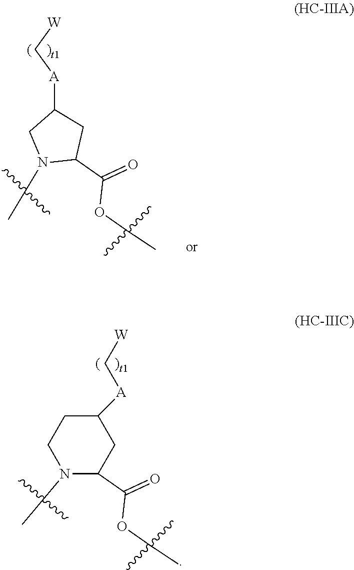

In some embodiments, in the above formulas,
A is absent, —O—, —N(R⁷)—, —OC(O)—, or —C(O)O—;
X is absent, —O—, or —C(O)—; and
Z is —O—, —C(O)O—, or —OC(O)—.

In some embodiments, the head group has a structure of formula

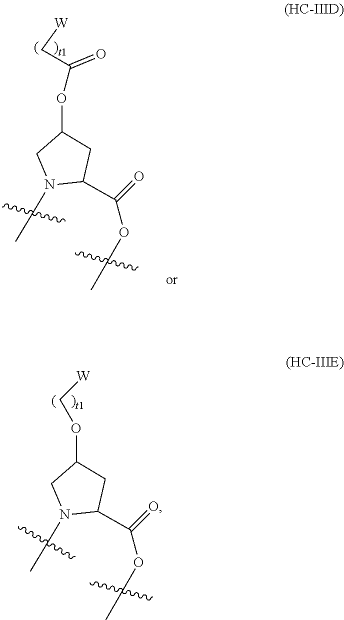

wherein t1 is 0, 1, 2, or 3.

In some embodiments, W is hydroxyl, substituted or unsubstituted hydroxyalkyl, or one of the following moieties:

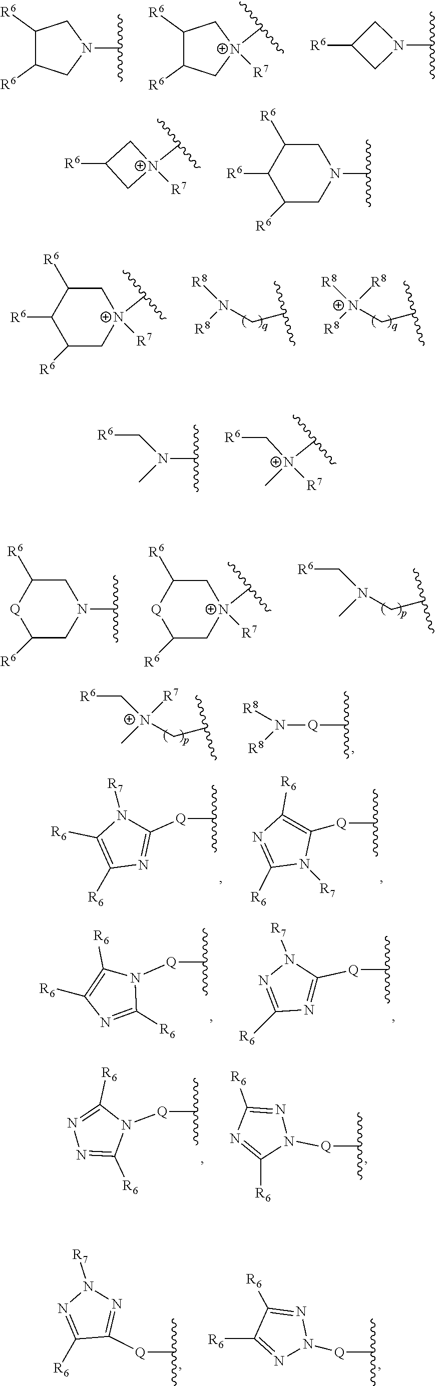

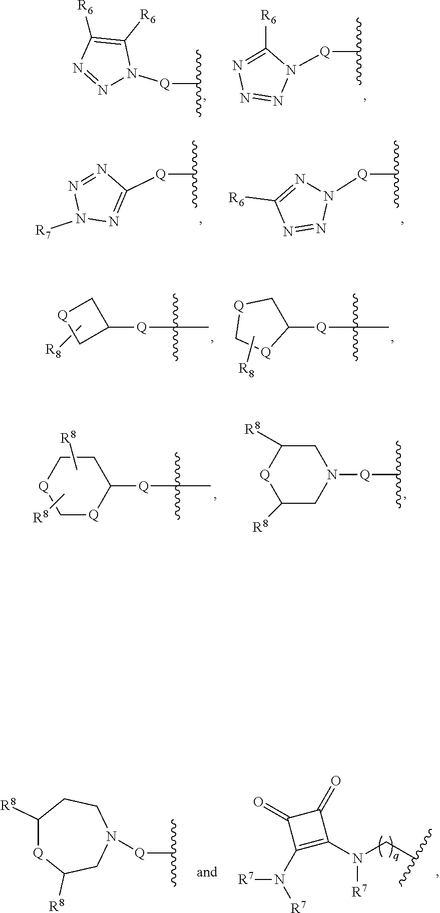

wherein
   each Q is independently absent, —O—, —C(O)—, —C(S)—, —C(O)O—, —(CH₂)_{q}—C(R⁷)₂—, —C(O)N(R⁷)—, —C(S)N(R⁷)—;
   R⁶ is independently H, alkyl, hydroxyl, hydroxyalkyl, alkoxy, —O-alkylene-O-alkyl, —O— alkylene-N(R⁷)₂, amino, alkylamino, aminoalkyl, thiol, thiolalkyl, or N(R⁷)₃-alkylene-Q-;
   each R⁸ is independently H, alkyl, hydroxyalkyl, amino, aminoalkyl, alkylamino, thiol, or thiolalkyl, heterocyclyl, heteroaryl, or two R⁸ together with the nitrogen atom may form a ring, optionally substituted with one or more alkyl, hydroxy, hydroxyalkyl, alkoxy, alkylaminoalkyl, alkylamino, aminoalkyl;
   q is 0, 1, 2, 3, 4, or 5; and
   p is 0, 1, 2, 3, 4, or 5.

In some embodiments, W is one of the following:

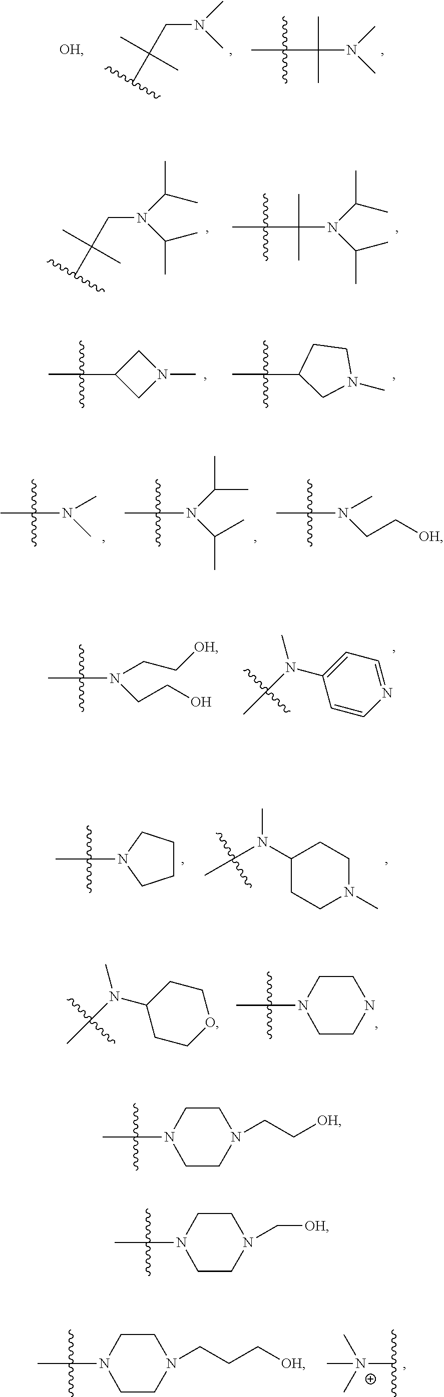

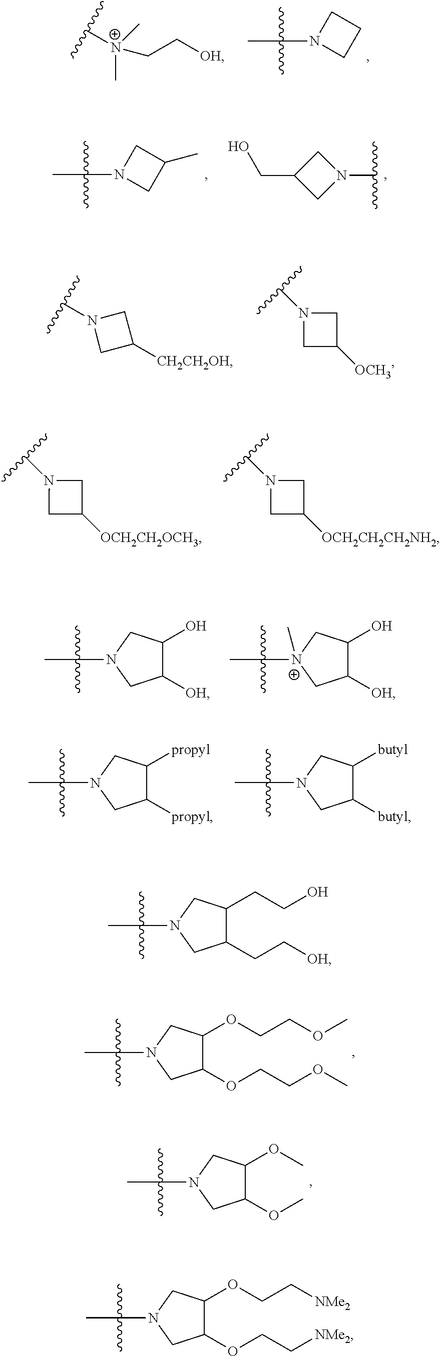

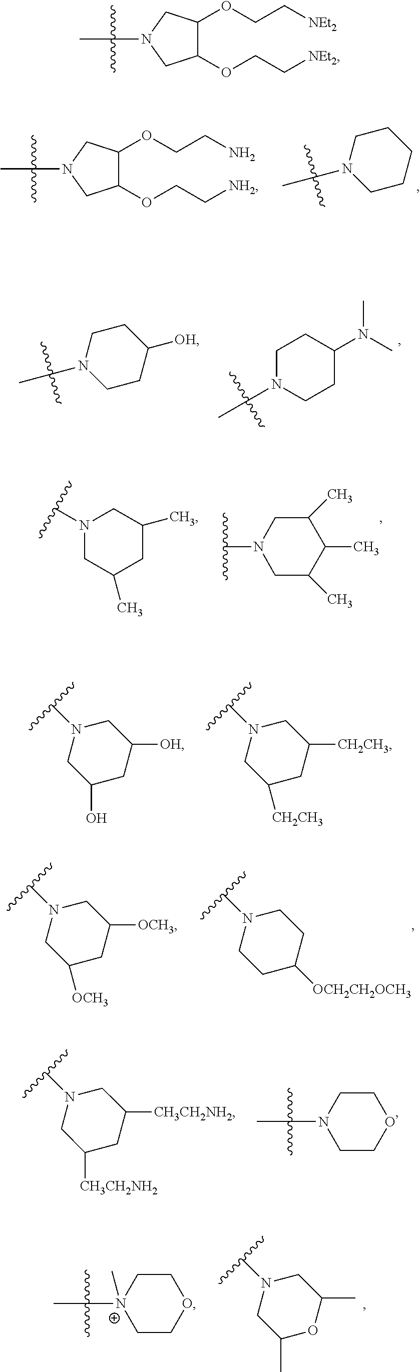

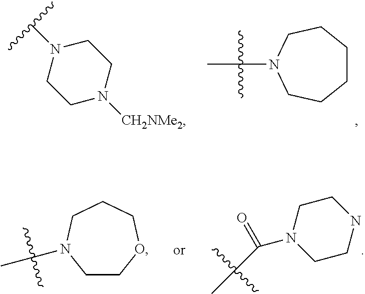

In some embodiments, the ionizable lipid is represented by formula of

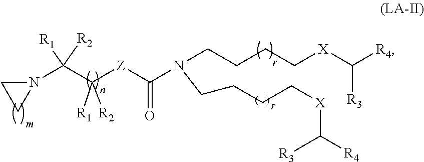

pharmaceutically acceptable salts thereof, and stereoisomers of any of the foregoing, wherein
R₁ is each independently H, C₁-C₃ alkyl, OH, halogen, SH, or NR₁₀R₁₁; R₁ and R₂ can be taken together to form a cyclic ring; R₁₀ and R₁₁ are each independently H, C₁-C₃ alkyl, and R₁₀ and R₁₁ can be taken together to form a heterocyclic ring;
R₂ is each independently H, C₁-C₃ alkyl, OH, halogen, SH, or NR₁₀R₁₁; R₁ and R₂ can be taken together to form a cyclic ring; R₁₀ and R₁₁ are each independently H, C₁-C₃ alkyl, and R₁₀ and R₁₁ can be taken together to form a heterocyclic ring;
m is 1, 2, 3, 4, 5, 6, 7 or 8;
n is 0, 1, 2, 3 or 4;
r is each independently 0, 1, 2, 3, 4, 5, 6, 7 or 8;
R₃ is each independently H, or C₃-C₁₀ alkyl;
R₄ is each independently H, or C₃-C₁₀ alkyl; provided that at least one of R₃ and R₄ is not H;
Z is absent, O, S, or NR₁₂; wherein R₁₂ is C₁-C₇ alkyl;
each X is independently X′,

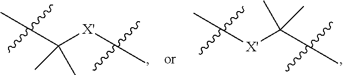

provided that at least one X in the formula is

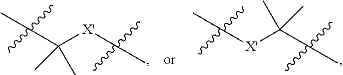

and
X′ is a biodegradable moiety.

In some embodiments, each X is

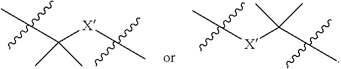

In some embodiments, X is —OCO—, —COO—, —NR⁷CO—, —CONR⁷—, —C(O—R₁₃)—O-(acetal), —COO(CH₂)ₛ—, —CONH(CH₂)ₛ—, —C(O—R₁₃)—O—(CH₂)ₛ—; wherein R⁷ is H or C₁-C₃ alkyl; and R₁₃ is C₃-C₁₀ alkyl.
In some embodiments, at least one X in the formula is

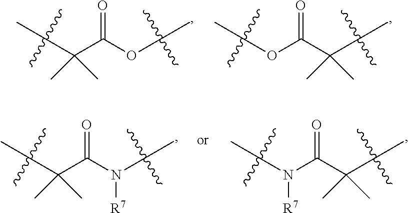

wherein R⁷ is H or methyl. In one embodiment, each X is

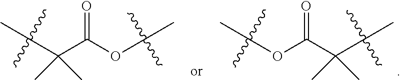

In one embodiment, each X is

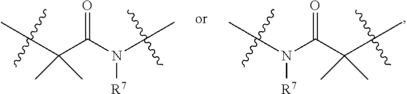

wherein R⁷ is H or methyl.

In some embodiments, m=3. In some embodiments, n=0 or 1. In some embodiments, each R R₁ and R₂ is H. In some embodiments, Z is absent.

In some embodiments, Z is S. In some embodiments, Z is O. In some embodiments, Z is NH.

In some embodiments, r is 3. In some embodiments, r is 4.

More embodiments of the above ionizable lipids comprising at least one head group (e.g., head group of formulas (HA-I) to (HA-VII), (HB—I), and (HC—I) to (HC-IIIE′)), and at least one tail group of formula (TI) or (T1′) (e.g., tail group of formula (TII), (TIII), TIV, TV, TII′, or TIII′), in the Ionizable lipid compounds group iv), may be found in PCT Application No. PCT/US23/31669, filed on Aug. 31, 2023, the content of which is incorporated herein by reference in its entirety. Moreover, all the ionizable lipids of formulas (LA-I)-(LA-VII), (LB-1)-(LB-VII), (LC-IA)-(LC-IC), (LC-IIA)-(LC-IIC), and (LC-IIIA)-(LC-IIIE) of PCT Application No. PCT/US23/31669, filed on Aug. 31, 2023 are suitable for use as the ionizable lipids in this disclosure, and are incorporated herein by reference in its entirety.

Certain exemplary ionizable lipid compounds disclosed herein are set forth in Table IV below.

TABLE IV
Exemplary ionizable lipid compounds.

| Lipid | | |
|---|---|---|
| No. | Structure | Name |
| 2243 | 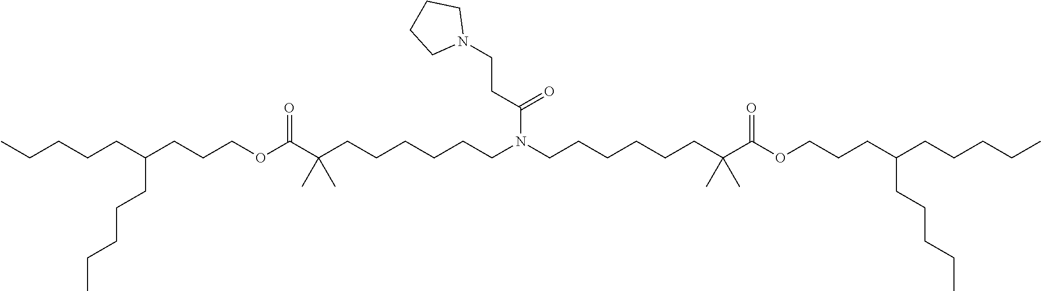 [SMILES: CCCCCC(CCCCC)CCCOC(=O)C(C)(C)CCCCCCN(CCCCCCC(C)(C)C(=O)OCCCC(CCCCC)CCCCC)C(=O)CCN1CCCC1] | bis(4-pentylnonyl) 8,8′-((3-(pyrrolidin-1- yl)propanoyl) azanediyl)bis(2,2- dimethyloctanoate) |
| 2330 | 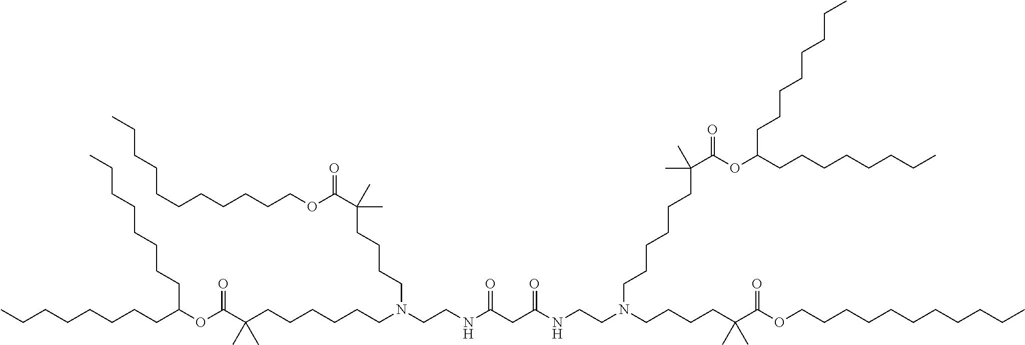 [SMILES: CCCCCCCCCCCOC(=O)C(C)(C)CCCCN(CCCCCCC(C)(C)C(=O)OC(CCCCCCCC)CCCCCCCC)CCNC(=O)CC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9,19-bis(5,5- dimethyl-6-oxo-6- (undecyloxy)hexyl)- 2,2,26,26- tetramethyl-13,15- dioxo-9,12,16,19- tetraazahepta- cosanedioate |
| 2331 | 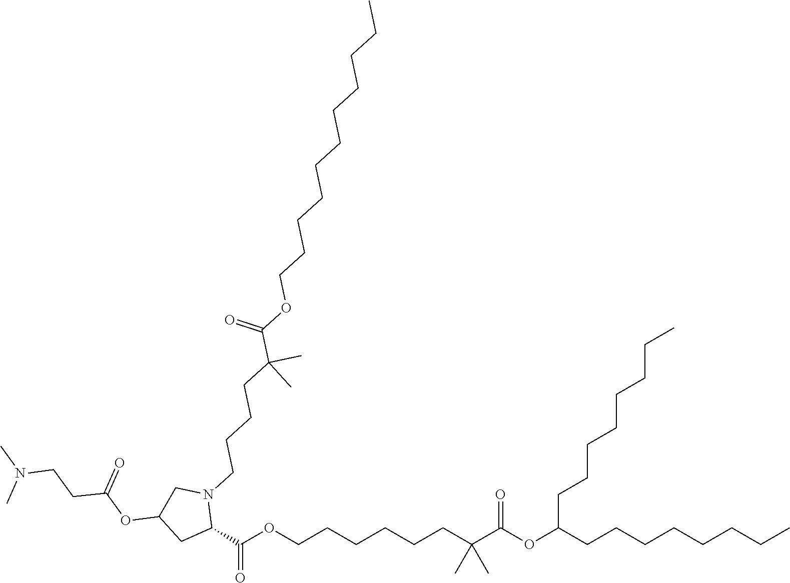 [SMILES: CCCCCCCCCCCOC(=O)C(C)(C)CCCCN1CC(OC(=O)CCN(C)C)C[C@H]1C(=O)OCCCCCCC(C)(C)C(=O)OC(CCCCCCCC)CCCCCCCC] | 8-(heptadecan-9- yloxy)-7,7-dimethyl- 8-oxooctyl (2S)-1- (5,5-dimethyl-6-oxo- 6- (undecyloxy)hexyl)- 4-((3- (dimethylamino) propanoyl)oxy) pyrrolidine- 2-carboxylate |
| 2333 | 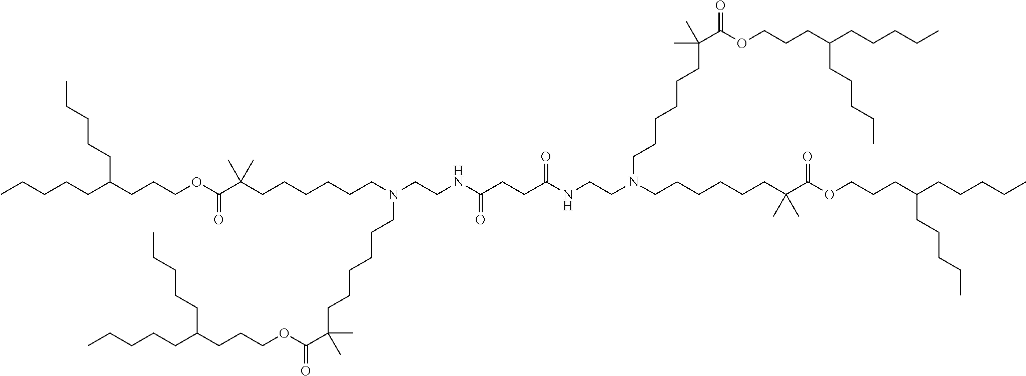 [SMILES: CCCCCC(CCCCC)CCCOC(=O)C(C)(C)CCCCCCN(CCCCCCC(C)(C)C(=O)OCCCC(CCCCC)CCCCC)CCNC(=O)CCC(=O)NCCN(CCCCCCC(C)(C)C(=O)OCCCC(CCCCC)CCCCC)CCCCCCC(C)(C)C(=O)OCCCC(CCCCC)CCCCC] | bis(4-pentylnonyl) 9,20-bis(7,7- dimethyl-8-oxo-8- ((4- pentylnonyl)oxy) octyl)- 2,2,27,27- tetramethyl-13,16- dioxo-9,12,17,20- tetraazaocta- cosanedioate |
| 2335 | 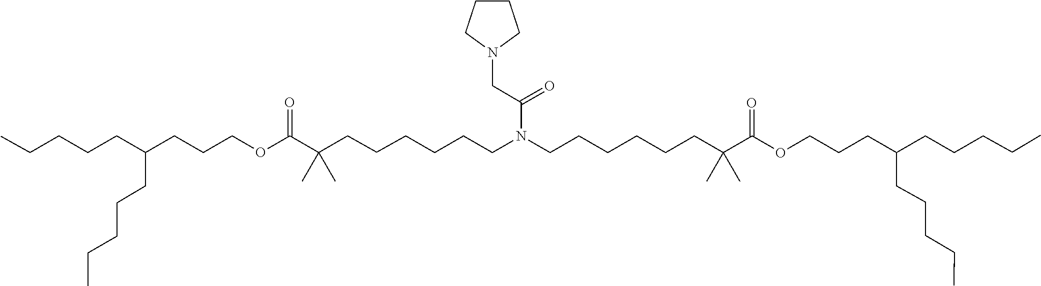 [SMILES: CCCCCC(CCCCC)CCCOC(=O)C(C)(C)CCCCCCN(CCCCCCC(C)(C)C(=O)OCCCC(CCCCC)CCCCC)C(=O)CN1CCCC1] | bis(4-pentylnonyl) 8,8′-((2-(pyrrolidin-1- yl)acetyl)azanediyl) bis(2,2- dimethyloctanoate) |
| 2365 | 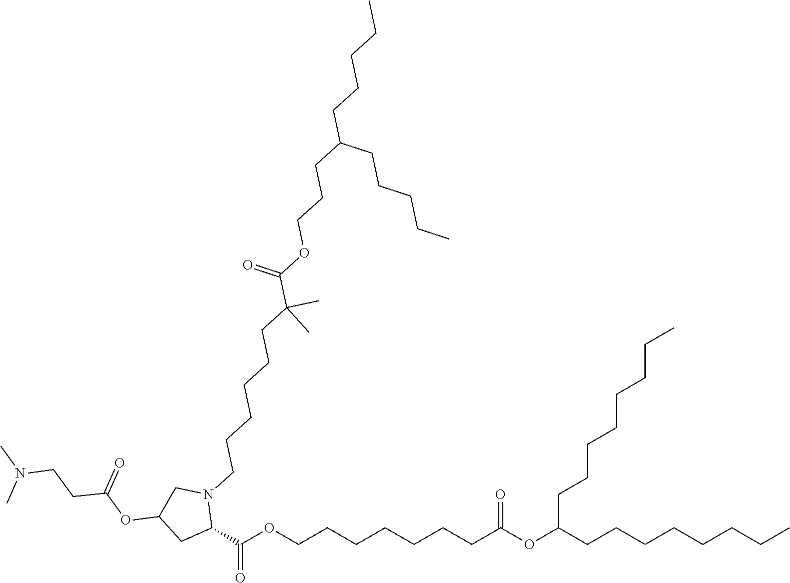 [SMILES: CCCCCCCCC(CCCCCCCC)OC(=O)CCCCCCCOC(=O)[C@@H]1CC(OC(=O)CCN(C)C)CN1CCCCCCC(C)(C)C(=O)OCCCC(CCCCC)CCCCC] | 8-(heptadecan-9- yloxy)-8-oxooctyl (2S)-1-(7,7-dimethyl- 8-oxo-8-((4- pentylnonyl)oxy) octyl)-4-((3- (dimethylamino) propanoyl)oxy) pyrrolidine- 2-carboxylate |
| 2366 | 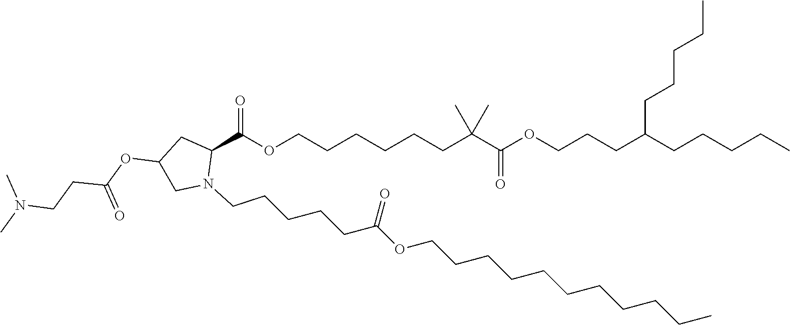 [SMILES: CCCCCCCCCCCOC(=O)CCCCCN1CC(OC(=O)CCN(C)C)C[C@H]1C(=O)OCCCCCCC(C)(C)C(=O)OCCCC(CCCCC)CCCCC] | 7,7-dimethyl-8-oxo- 8-((4- pentylnonyl)oxy) octyl (2S)-4-((3- (dimethylamino) propanoyl)oxy)- 1-(6-oxo-6- (undecyloxy)hexyl) pyrrolidine-2- carboxylate |
| 2367 | 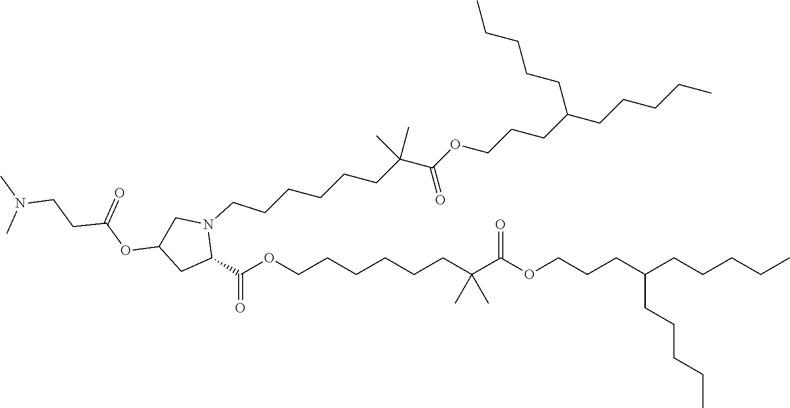 [SMILES: CCCCCC(CCCCC)CCCOC(=O)C(C)(C)CCCCCCOC(=O)[C@@H]1CC(OC(=O)CCN(C)C)CN1CCCCCCC(C)(C)C(=O)OCCCC(CCCCC)CCCCC] | 7,7-dimethyl-8-oxo- 8-((4- pentylnonyl)oxy) octyl (2S)-1-(7,7- dimethyl-8-oxo-8- ((4- pentylnonyl)oxy) octyl)-4-((3- (dimethylamino) propanoyl)oxy) pyrrolidine- 2-carboxylate |
| 2368 | 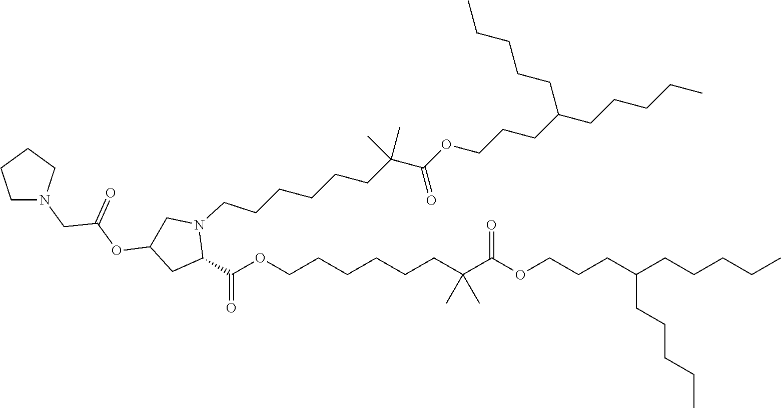 [SMILES: CCCCCC(CCCCC)CCCOC(=O)C(C)(C)CCCCCCOC(=O)[C@@H]1CC(OC(=O)CN2CCCC2)CN1CCCCCCC(C)(C)C(=O)OCCCC(CCCCC)CCCCC] | 7,7-dimethyl-8-oxo- 8-((4- pentylnonyl)oxy) octyl (2S)-1-(7,7- dimethyl-8-oxo-8- ((4- pentylnonyl)oxy) octyl)-4-(2- (pyrrolidin-1- yl)acetoxy)pyrrolidine- 2-carboxylate |
| 2369 | 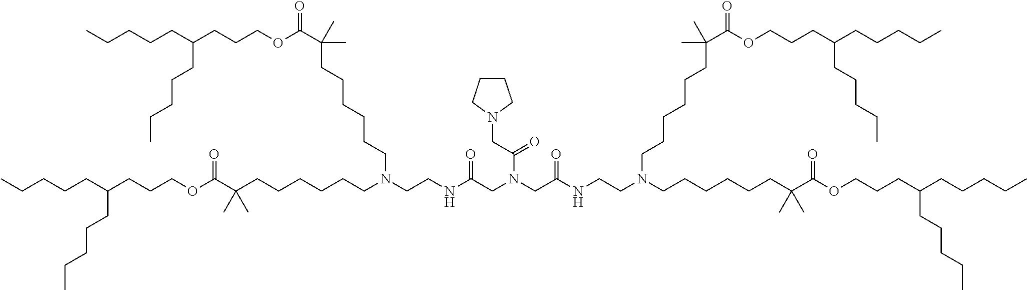 [SMILES: CCCCCC(CCCCC)CCCOC(=O)C(C)(C)CCCCCCN(CCCCCCC(C)(C)C(=O)OCCCC(CCCCC)CCCCC)CCNC(=O)CN(CC(=O)NCCN(CCCCCCC(C)(C)C(=O)OCCCC(CCCCC)CCCCC)CCCCCCC(C)(C)C(=O)OCCCC(CCCCC)CCCCC)C(=O)CN1CCCC1] | bis(4-pentylnonyl) 9,21-bis(7,7- dimethyl-8-oxo-8- ((4- pentylnonyl)oxy) octyl)-2,2,28,28- tetramethyl-13,17- dioxo-15-(2- (pyrrolidin-1- yl)acetyl)- 9,12,15,18,21- pentaazanona- cosanedioate |
| 2383 | 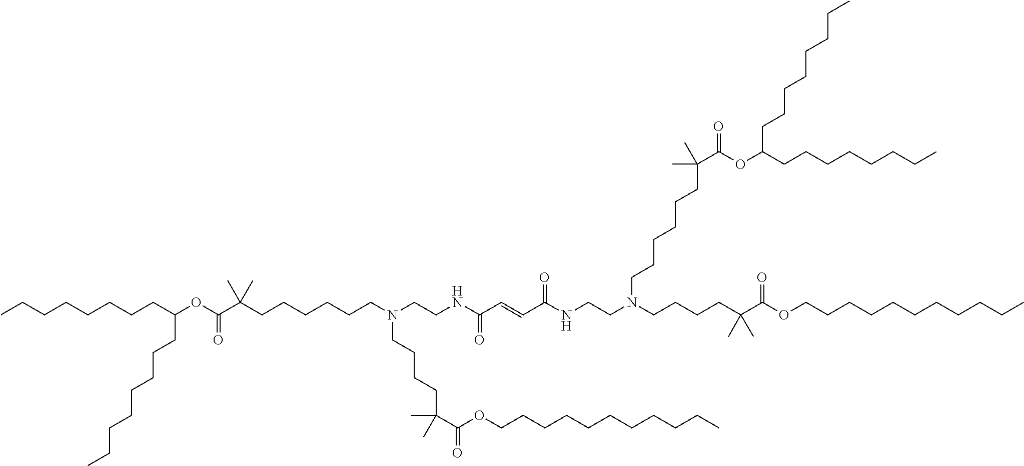 [SMILES: CCCCCCCCCCCOC(=O)C(C)(C)CCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) (E)-9,20-bis(5,5- dimethyl-6-oxo-6- (undecyloxy)hexyl)- 2,2,27,27- tetramethyl-13,16- dioxo-9,12,17,20- tetraazaoctacos-14- enedioate |
| 2392 | 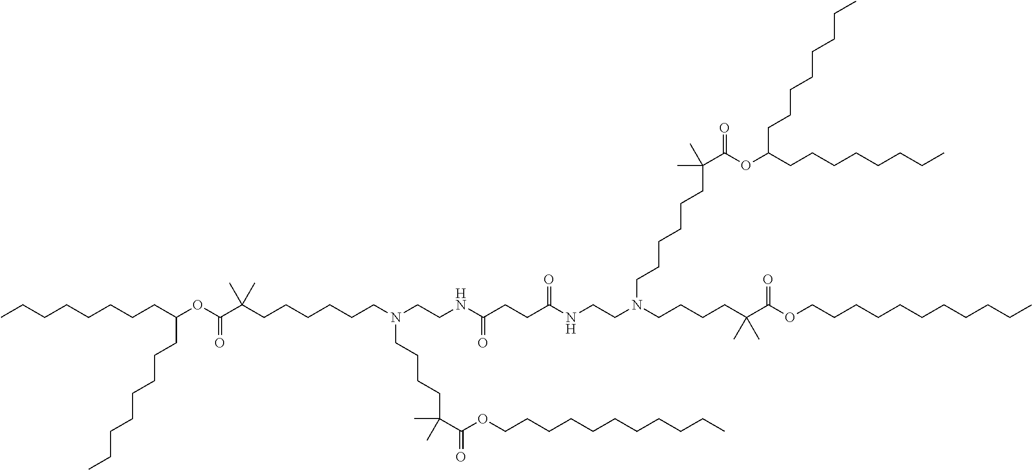 [SMILES: CCCCCCCCCCCOC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9,20-bis(5,5- dimethyl-6-oxo-6- (undecyloxy)hexyl)- 2,2,27,27- tetramethyl-13,16- dioxo-9,12,17,20- tetraazaocta- cosanedioate |
| 2398 | 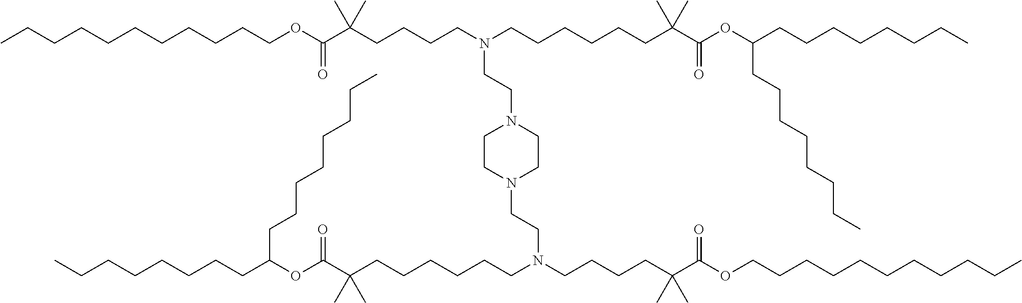 | di(heptadecan-9-yl) 8,8′-((piperazine-1,4- diylbis(ethane-2,1- diyl))bis((5,5- dimethyl-6-oxo-6- (undecyloxy)hexyl) azanediyl))bis(2,2- dimethyloctanoate) |
| 2424 | 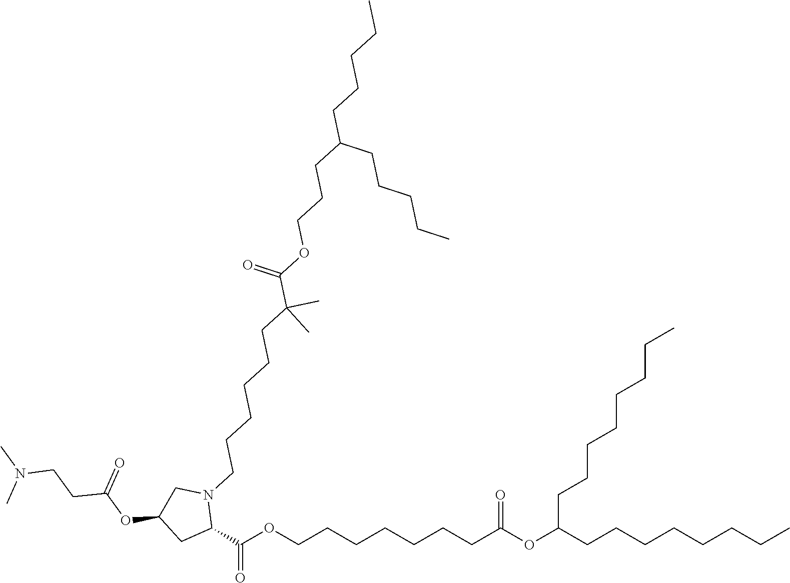 [SMILES: CCCCCCCCC(CCCCCCCC)OC(=O)CCCCCCCOC(=O)[C@@H]1C[C@@H](OC(=O)CCN(C)C)CN1CCCCCCC(C)(C)C(=O)OCCCC(CCCCC)CCCCC] | 8-(heptadecan-9- yloxy)-8-oxooctyl (2S,4R)-1-(7,7- dimethyl-8-oxo-8- ((4- pentylnonyl)oxy) octyl)-4-((3- (dimethylamino) propanoyl)oxy) pyrrolidine- 2-carboxylate |
| 2425 | 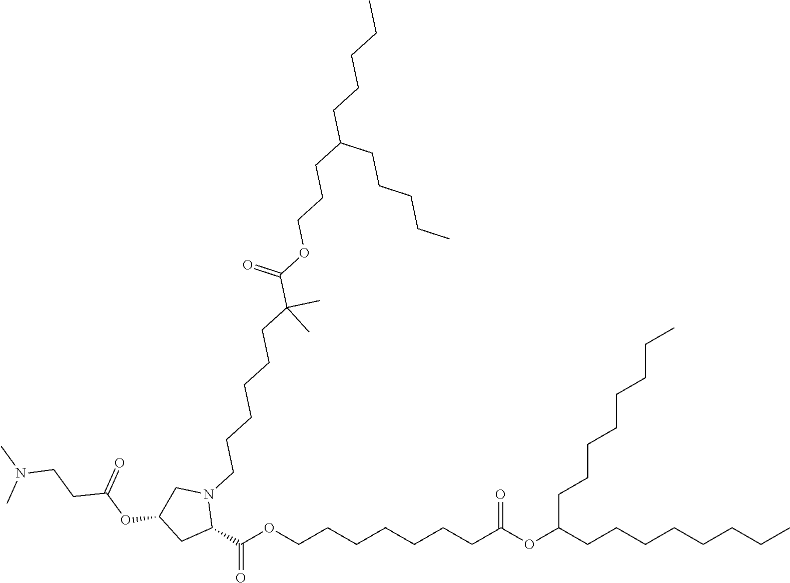 [SMILES: CCCCCCCCC(CCCCCCCC)OC(=O)CCCCCCCOC(=O)[C@@H]1C[C@H](OC(=O)CCN(C)C)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((3- (dimethylamino) propanoyl)oxy) pyrrolidine- 2-carboxylate |
| 2432 | 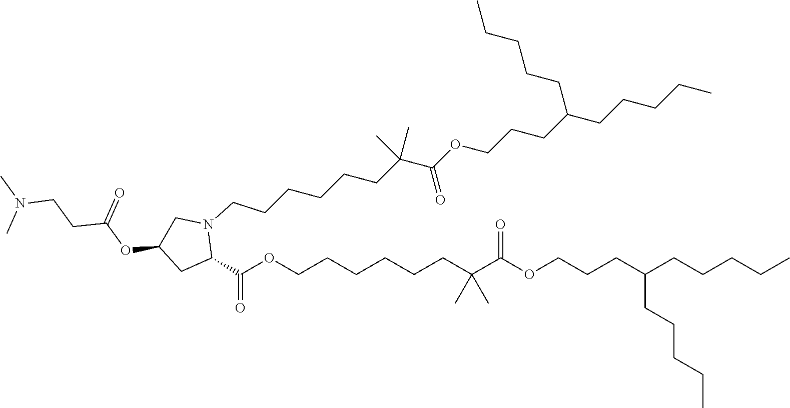 [SMILES: CCCCCC(CCCCC)CCCOC(=O)C(C)(C)CCCCCCOC(=O)[C@@H]1C[C@@H](OC(=O)CCN(C)C)CN1CCCCCCC(C)(C)C(=O)OCCCC(CCCCC)CCCCC] | 7,7-dimethyl-8-oxo- 8-((4- pentylnonyl)oxy)octyl (2S,4R)-1-(7,7- dimethyl-8-oxo-8- ((4- pentylnonyl)oxy) octyl)-4-((3- (dimethylamino) propanoyl)oxy) pyrrolidine- 2-carboxylate |
| 2433 | 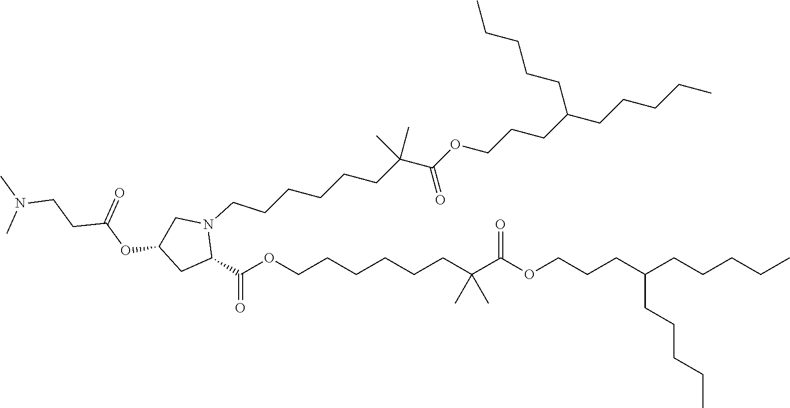 [SMILES: CCCCCC(CCCCC)CCCOC(=O)C(C)(C)CCCCCCOC(=O)[C@@H]1C[C@H](OC(=O)CCN(C)C)CN1CCCCCCC(C)(C)C(=O)OCCCC(CCCCC)CCCCC] | 7,7-dimethyl-8-oxo- 8-((4- pentylnonyl)oxy)octyl (2S,4S)-1-(7,7- dimethyl-8-oxo-8- ((4- pentylnonyl)oxy) octyl)-4-((3- (dimethylamino) propanoyl)oxy) pyrrolidine- 2-carboxylate |
| 2441 | 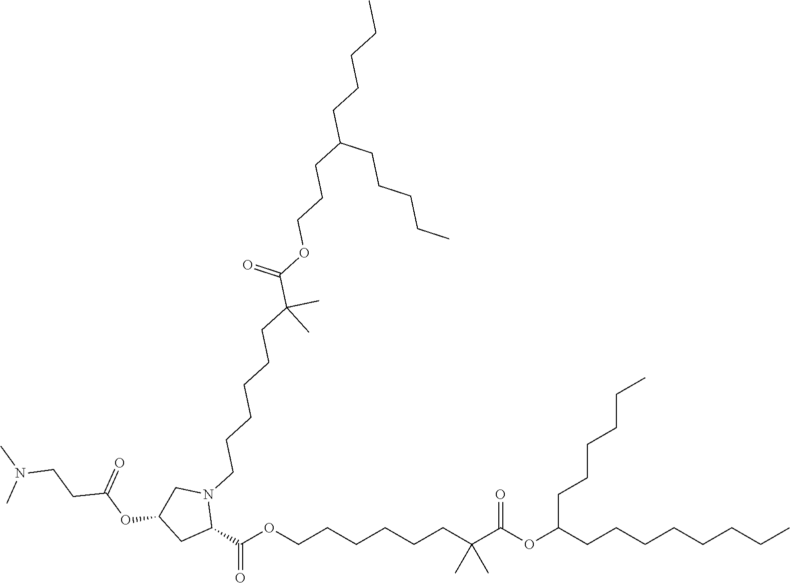 [SMILES: CCCCCCCCC(CCCCCC)OC(=O)C(C)(C)CCCCCCOC(=O)[C@@H]1C[C@H](OC(=O)CCN(C)C)CN1CCCCCCC(C)(C)C(=O)OCCCC(CCCCC)CCCCC] | 7,7-dimethyl-8-oxo- 8-(pentadecan-7- yloxy)octyl (2S,4S)- 1-(7,7-dimethyl-8- oxo-8-((4- pentylnonyl)oxy) octyl)-4-((3- (dimethylamino) propanoyl)oxy) pyrrolidine- 2-carboxylate |
| 2442 | 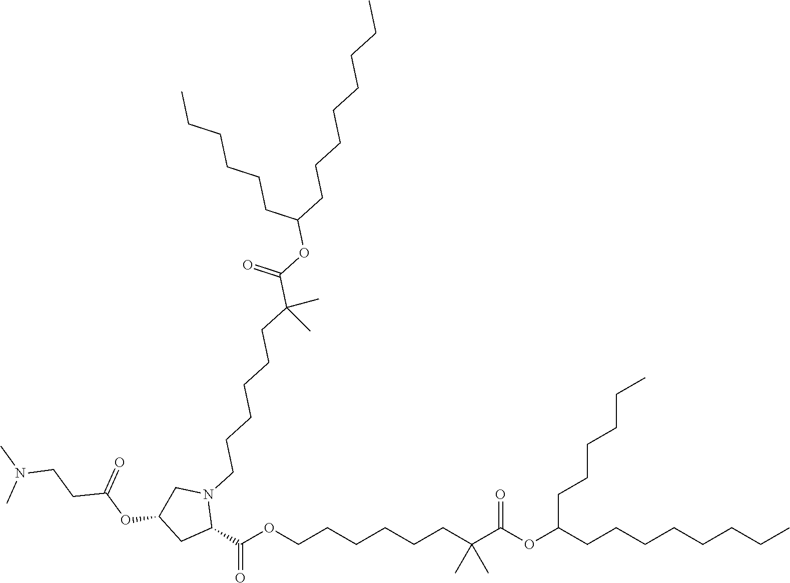 [SMILES: CCCCCCCCC(CCCCCC)OC(=O)C(C)(C)CCCCCCOC(=O)[C@@H]1C[C@H](OC(=O)CCN(C)C)CN1CCCCCCC(C)(C)C(=O)OC(CCCCCC)CCCCCCCC] | 7,7-dimethyl-8-oxo- 8-(pentadecan-7- yloxy)octyl (2S,4S)- 1-(7,7-dimethyl-8- oxo-8-(pentadecan- 7-yloxy)octyl)-4-((3- (dimethylamino) propanoyl)oxy) pyrrolidine- 2-carboxylate |
| 2443 | 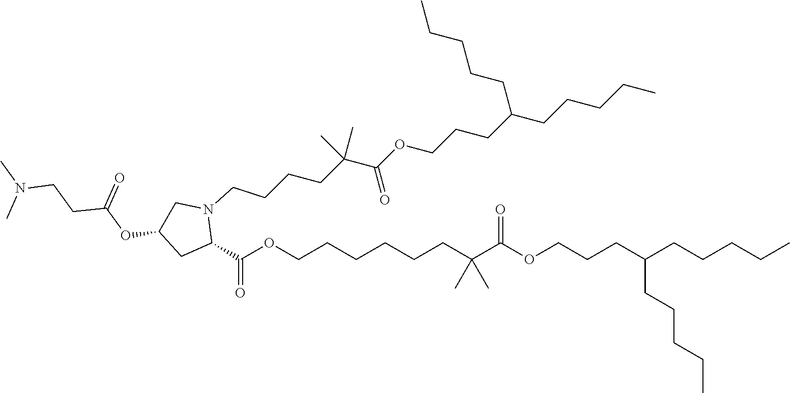 [SMILES: CCCCCC(CCCCC)CCCOC(=O)C(C)(C)CCCCCCOC(=O)[C@@H]1C[C@H](OC(=O)CCN(C)C)CN1CCCCC(C)(C)C(=O)OCCCC(CCCCC)CCCCC] | 7,7-dimethyl-8-oxo- 8-((4- pentylnonyl)oxy)octyl (2S,4S)-1-(5,5- dimethyl-6-oxo-6- ((4- pentylnonyl)oxy) hexyl)-4-((3- (dimethylamino) propanoyl)oxy) pyrrolidine- 2-carboxylate |
| 2454 | 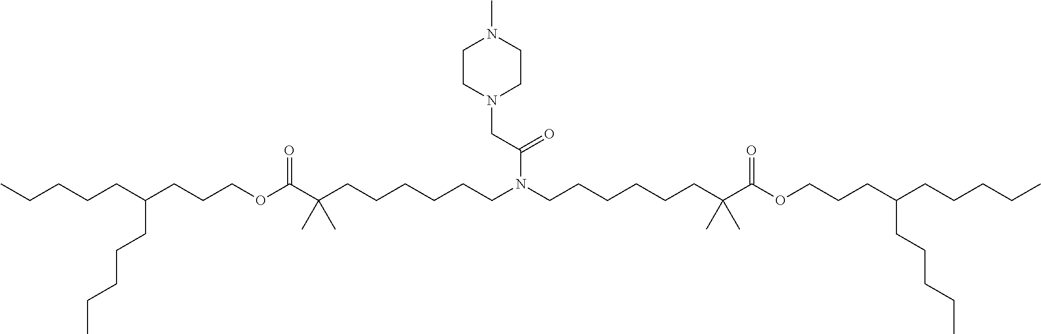 [SMILES: CCCCCC(CCCCC)CCCOC(=O)C(C)(C)CCCCCCN(CCCCCCC(C)(C)C(=O)OCCCC(CCCCC)CCCCC)C(=O)CN1CCN(C)CC1] | bis(4-pentylnonyl) 8,8′-((2-(4- methylpiperazin-1- yl)acetyl)azanediyl) bis(2,2- dimethyloctanoate) |
| 2481 | 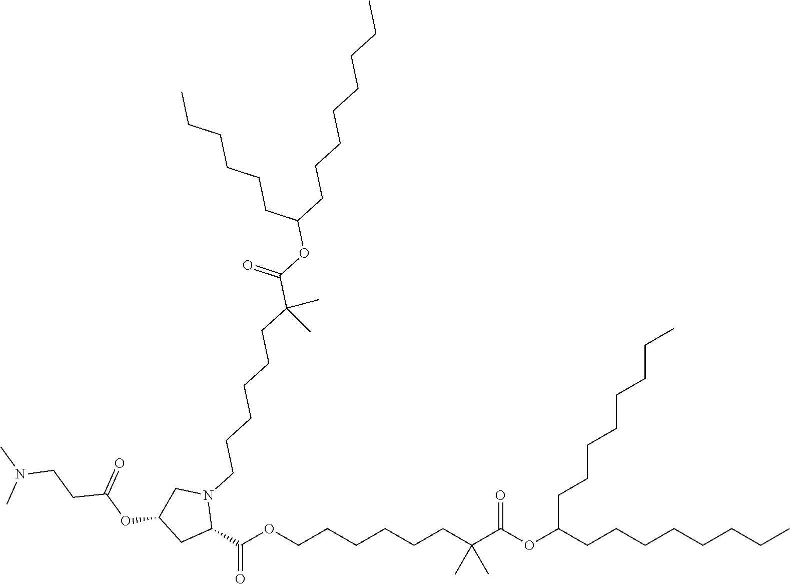 [SMILES: CCCCCCCCC(CCCCCC)OC(=O)C(C)(C)CCCCCCN1C[C@@H](OC(=O)CCN(C)C)C[C@H]1C(=O)OCCCCCCC(C)(C)C(=O)OC(CCCCCCCC)CCCCCCCC] | 8-(heptadecan-9- yloxy)-7,7-dimethyl- 8-oxooctyl (2S,4S)- 1-(7,7-dimethyl-8- oxo-8-(pentadecan- 7-yloxy)octyl)-4-((3- (dimethylamino) propanoyl)oxy) pyrrolidine- 2-carboxylate |
| 2483 | 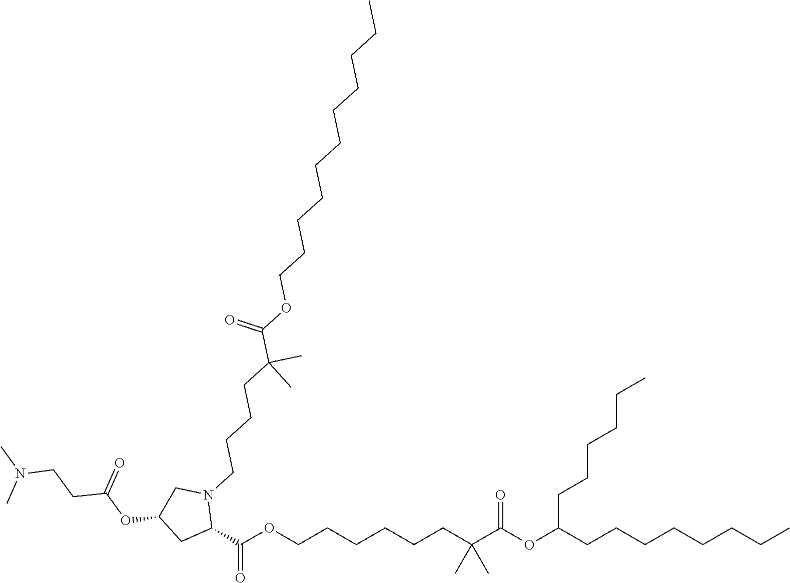 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCC)CCCCCCCC] | 7,7-dimethyl-8-oxo- 8-(pentadecan-7- yloxy)octyl (2S,4S)- 1-(5,5-dimethyl-6- oxo-6- (undecyloxy)hexyl)- 4-((3- (dimethylamino) propanoyl)oxy) pyrrolidine- 2-carboxylate |
| 2370 | 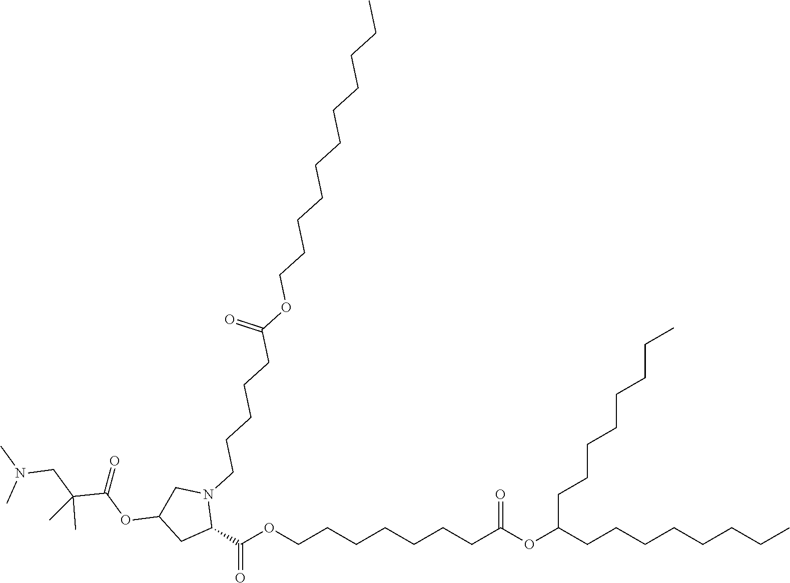 [SMILES: CCCCCCCCCCCOC(=O)CCCCCN1CC(OC(=O)C(C)(C)CN(C)C)C[C@H]1C(=O)OCCCCCCCC(=O)OC(CCCCCCCC)CCCCCCCC] | 8-(heptadecan-9- yloxy)-8-oxooctyl (2S)-4-((3- (dimethylamino)-2,2- dimethylpropanoyl) oxy)-1-(6-oxo-6- (undecyloxy)hexyl) pyrrolidine-2- carboxylate |
| 2511 | 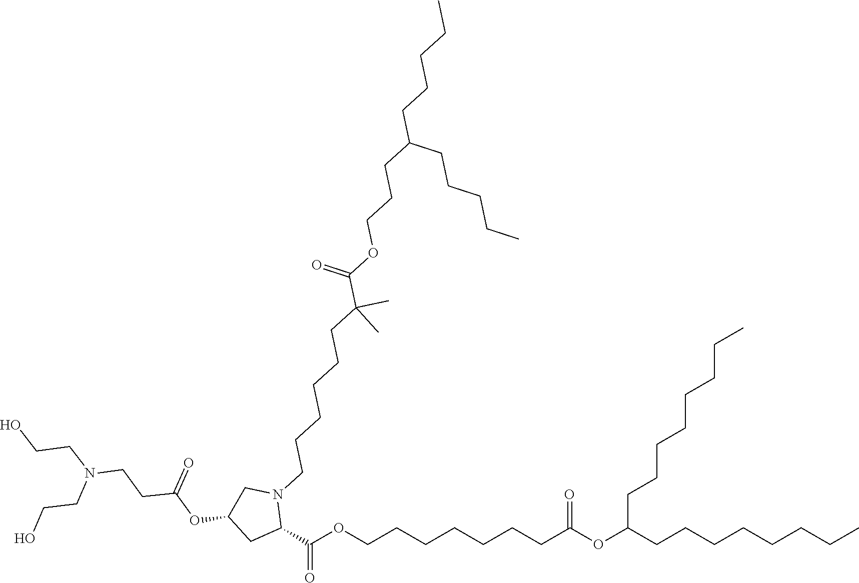 [SMILES: CCCCCCCCC(CCCCCCCC)OC(=O)CCCCCCCOC(=O)[C@@H]1C[C@H](OC(=O)CCN(CCO)CCO)CN1CCCCCCC(C)(C)C(=O)OCCCC(CCCCC)CCCCC] | 8-(heptadecan-9- yloxy)-8-oxooctyl (2S,4S)-4-((3-(bis(2- hydroxyethyl)amino) propanoyl)oxy)-1- (7,7-dimethyl-8-oxo- 8-((4- pentylnonyl)oxy) octyl)pyrrolidine-2- carboxylate |
| 2510 | 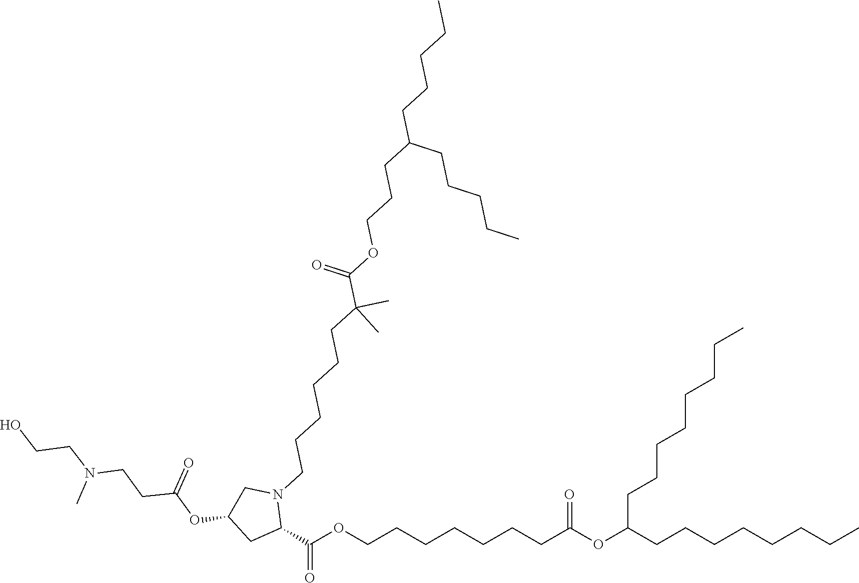 [SMILES: CCCCCCCCC(CCCCCCCC)OC(=O)CCCCCCCOC(=O)[C@@H]1C[C@H](OC(=O)CCN(C)CCO)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((3-((2- hydroxyethyl)(methyl) amino)propanoyl) oxy)pyrrolidine-2- carboxylate |
| 2509 | 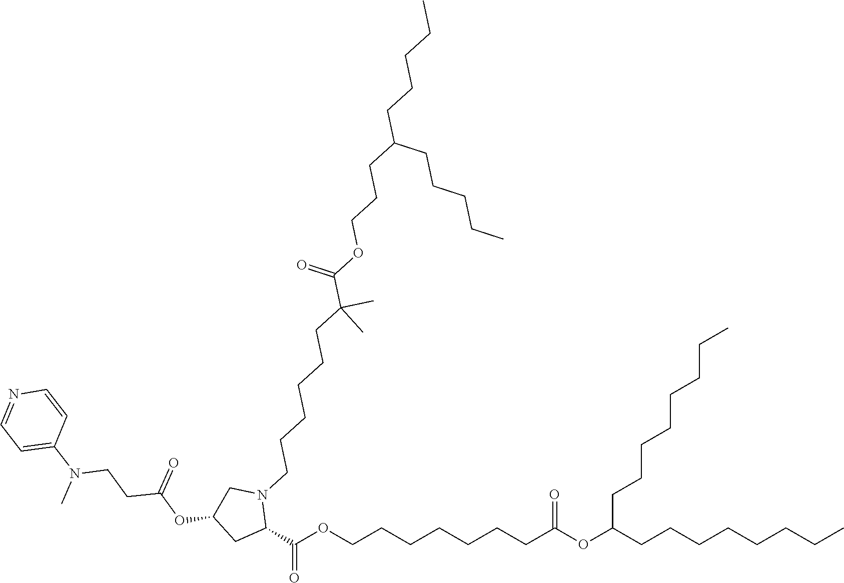 [SMILES: CCCCCCCCC(CCCCCCCC)OC(=O)CCCCCCCOC(=O)[C@@H]1C[C@H](OC(=O)CCN(C)c2ccncc2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((3- (methyl(pyridin-4- yl)amino)propanoyl) oxy)pyrrolidine-2- carboxylate |
| 2508 | 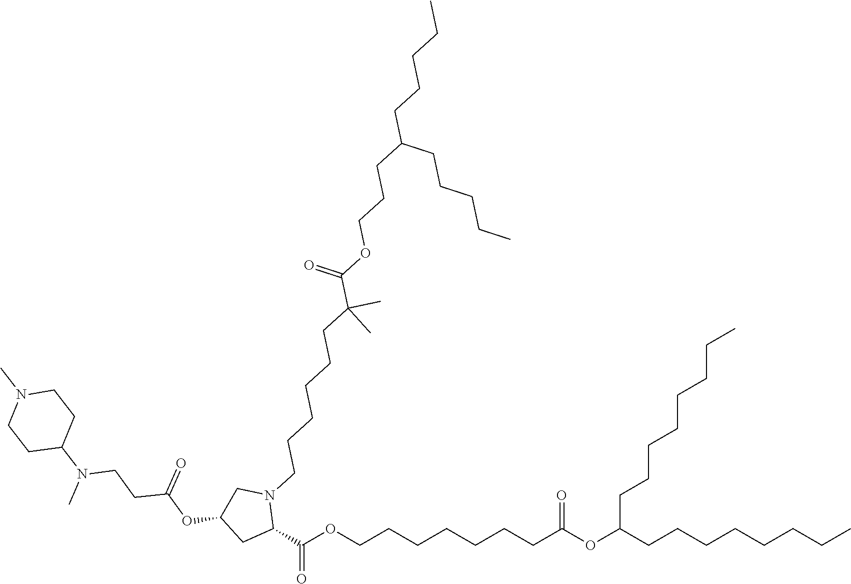 [SMILES: CCCCCCCCC(CCCCCCCC)OC(=O)CCCCCCCOC(=O)[C@@H]1C[C@H](OC(=O)CCN(C)C2CCN(C)CC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((3-(methyl(1- methylpiperidin-4- yl)amino)propanoyl) oxy)pyrrolidine-2- carboxylate |
| 2507 | 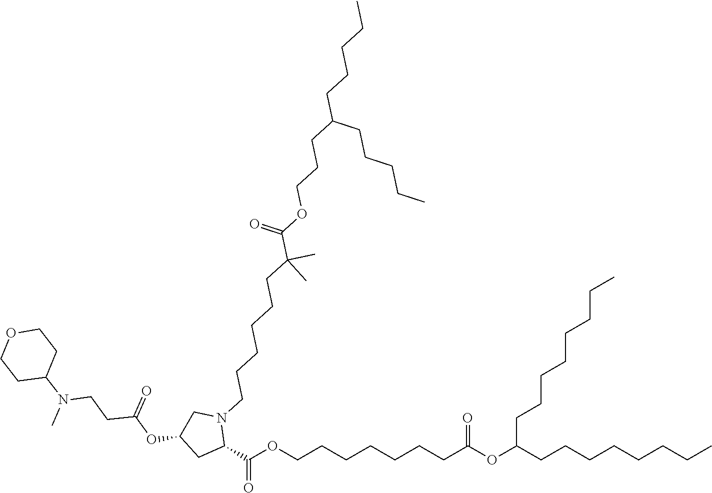 [SMILES: CCCCCCCCC(CCCCCCCC)OC(=O)CCCCCCCOC(=O)[C@@H]1C[C@H](OC(=O)CCN(C)C2CCOCC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((3- (methyl(tetrahydro- 2H-pyran-4- yl)amino)propanoyl) oxy)pyrrolidine-2- carboxylate |
| 2506 | 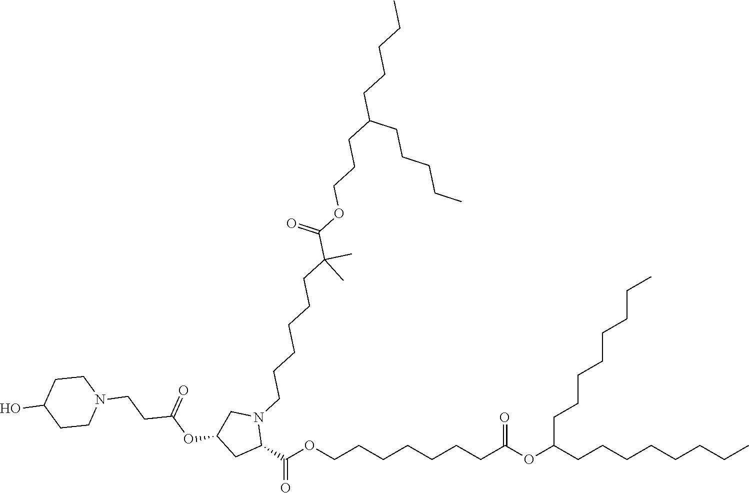 [SMILES: CCCCCCCCC(CCCCCCCC)OC(=O)CCCCCCCOC(=O)[C@@H]1C[C@H](OC(=O)CCN2CCC(O)CC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((3-(4- hydroxypiperidin-1- yl)propanoyl)oxy) pyrrolidine-2- carboxylate |
| 2505 | 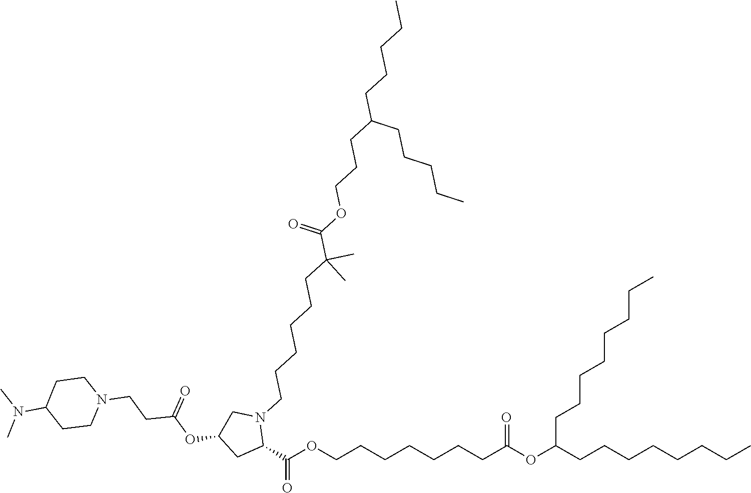 [SMILES: CCCCCCCCC(CCCCCCCC)OC(=O)CCCCCCCOC(=O)[C@@H]1C[C@H](OC(=O)CCN2CCC(N(C)C)CC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((3-(4- (dimethylamino) piperidin-1- yl)propanoyl)oxy) pyrrolidine-2- carboxylate |
| 2504 | 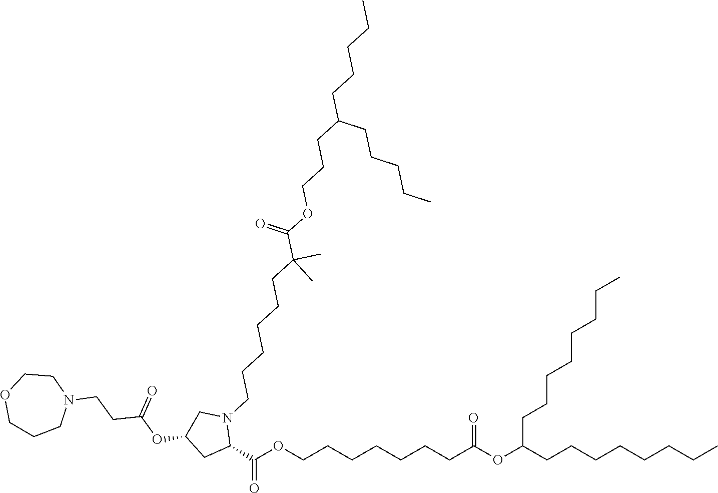 [SMILES: CCCCCCCCC(CCCCCCCC)OC(=O)CCCCCCCOC(=O)[C@@H]1C[C@H](OC(=O)CCN2CCCOCC2)CN1CCCCCCC(C)(C)C(=O)OCCCC(CCCCC)CCCCC] | 8-(heptadecan-9- yloxy)-8-oxooctyl (2S,4S)-4-((3-(1,4- oxazepan-4- yl)propanoyl)oxy)-1- (7,7-dimethyl-8-oxo- 8-((4- pentylnonyl)oxy) octyl)pyrrolidine-2- carboxylate |
| 2503 | 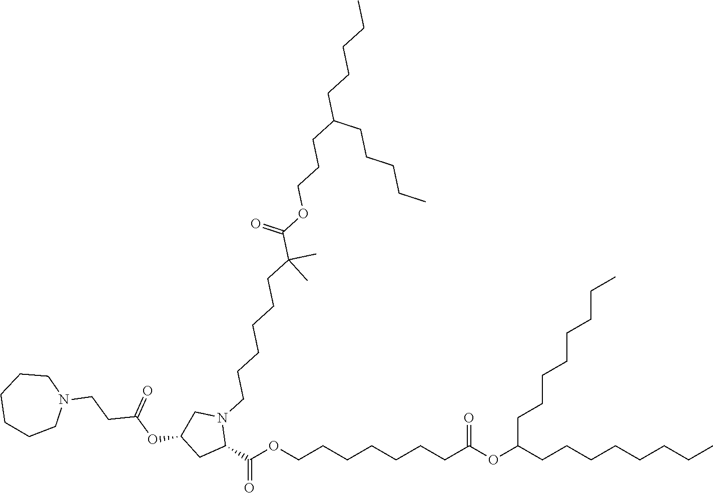 [SMILES: CCCCCCCCC(CCCCCCCC)OC(=O)CCCCCCCOC(=O)[C@@H]1C[C@H](OC(=O)CCN2CCCCCC2)CN1CCCCCCC(C)(C)C(=O)OCCCC(CCCCC)CCCCC] | 8-(heptadecan-9- yloxy)-8-oxooctyl (2S,4S)-4-((3- (azepan-1- yl)propanoyl)oxy)-1- (7,7-dimethyl-8-oxo- 8-((4- pentylnonyl)oxy) octyl)pyrrolidine-2- carboxylate |
| 2502 | 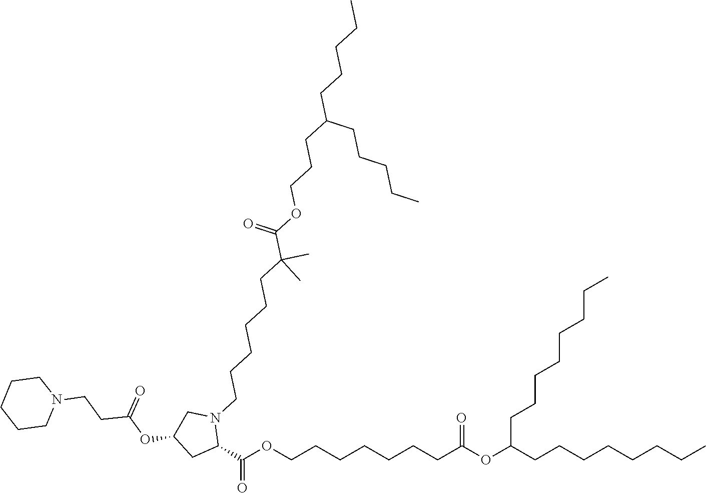 [SMILES: CCCCCCCCC(CCCCCCCC)OC(=O)CCCCCCCOC(=O)[C@@H]1C[C@H](OC(=O)CCN2CCCCC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((3- (piperidin-1- yl)propanoyl)oxy) pyrrolidine-2- carboxylate |
| 2501 | 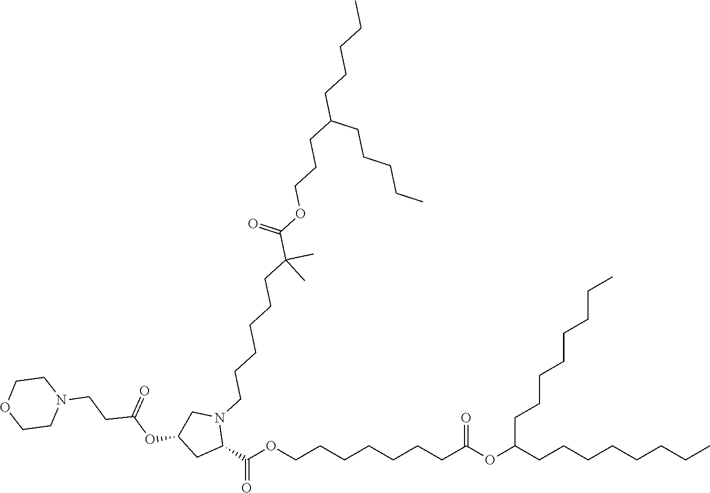 [SMILES: CCCCCCCCC(CCCCCCCC)OC(=O)CCCCCCCOC(=O)[C@@H]1C[C@H](OC(=O)CCN2CCOCC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((3- morpholinopropanoyl) oxy)pyrrolidine-2- carboxylate |
| 2500 | 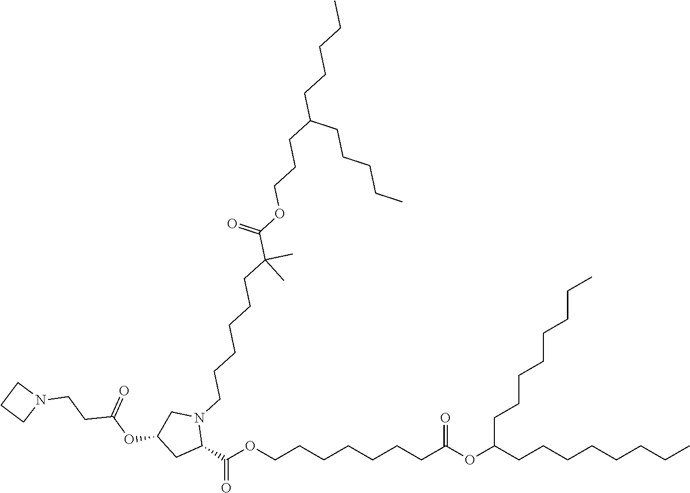 [SMILES: CCCCCCCCC(CCCCCCCC)OC(=O)CCCCCCCOC(=O)[C@@H]1C[C@H](OC(=O)CCN2CCC2)CN1CCCCCCC(C)(C)C(=O)OCCCC(CCCCC)CCCCC] | 8-(heptadecan-9- yloxy)-8-oxooctyl (2S,4S)-4-((3- (azetidin-1- yl)propanoyl)oxy)-1- (7,7-dimethyl-8-oxo- 8-((4- pentylnonyl)oxy) octyl)pyrrolidine-2- carboxylate |
| 2499 | 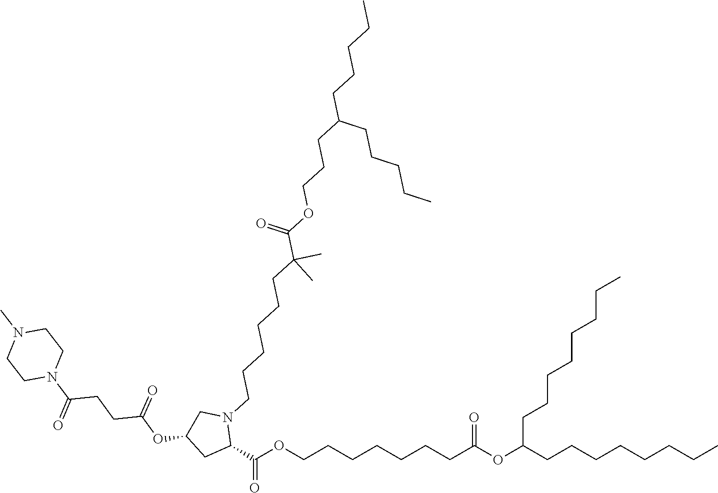 [SMILES: CCCCCCCCC(CCCCCCCC)OC(=O)CCCCCCCOC(=O)[C@@H]1C[C@H](OC(=O)CCC(=O)N2CCN(C)CC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((4-(4- methylpiperazin-1- yl)-4- oxobutanoyl)oxy) pyrrolidine-2- carboxylate |
| 2498 | 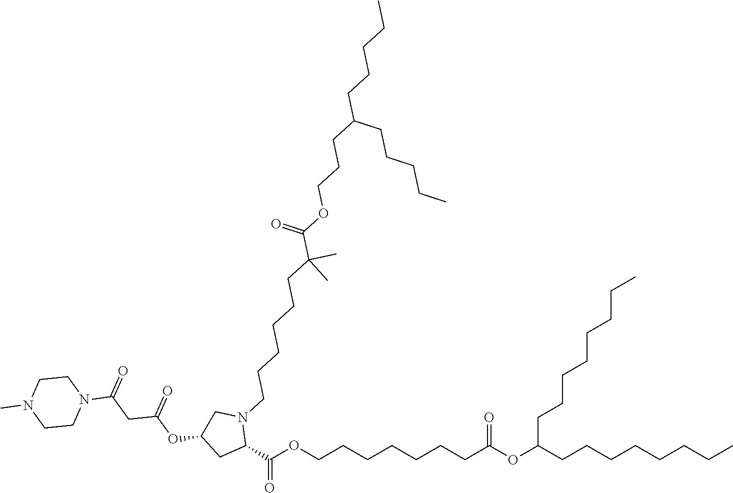 [SMILES: CCCCCCCCC(CCCCCCCC)OC(=O)CCCCCCCOC(=O)[C@@H]1C[C@H](OC(=O)CC(=O)N2CCN(C)CC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((3-(4- methylpiperazin-1- yl)-3- oxopropanoyl)oxy) pyrrolidine-2- carboxylate |
| 2488 | 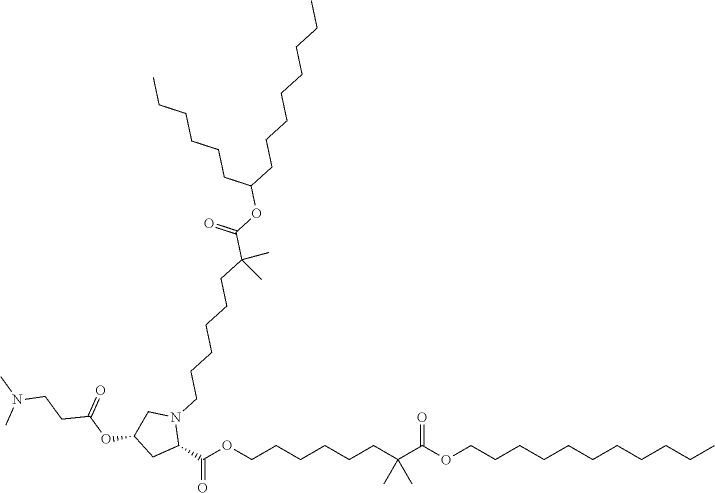 [SMILES: CCCCCCCCCCCOC(=O)C(C)(C)CCCCCCOC(=O)[C@@H]1C[C@H](OC(=O)CCN(C)C)CN1CCCCCCC(C)(C)C(=O)OC(CCCCCC)CCCCCCCC] | 7,7-dimethyl-8-oxo- 8-(undecyloxy)octyl (2S,4S)-1-(7,7- dimethyl-8-oxo-8- (pentadecan-7- yloxy)octyl)-4-((3- (dimethylamino) propanoyl)oxy) pyrrolidine- 2-carboxylate |
| 2487 | 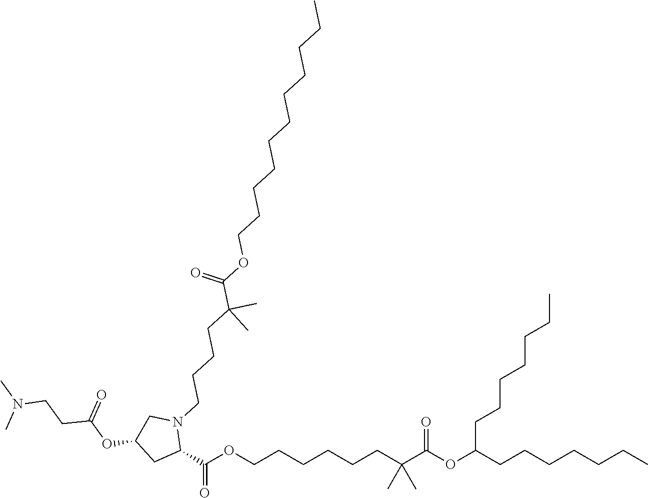 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCCC)CCCCCCC] | 7,7-dimethyl-8-oxo- 8-(pentadecan-8- yloxy)octyl (2S,4S)- 1-(5,5-dimethyl-6- oxo-6- (undecyloxy)hexyl)- 4-((3- (dimethylamino) propanoyl)oxy) pyrrolidine- 2-carboxylate |
| 2486 | 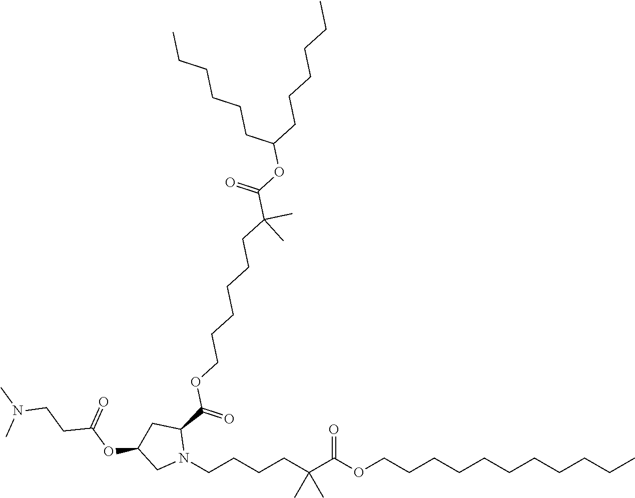 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCC)CCCCCC] | 7,7-dimethyl-8-oxo- 8-(tridecan-7- yloxy)octyl (2S,4S)- 1-(5,5-dimethyl-6- oxo-6- (undecyloxy)hexyl)- 4-((3- (dimethylamino) propanoyl)oxy) pyrrolidine- 2-carboxylate |
| 2485 | 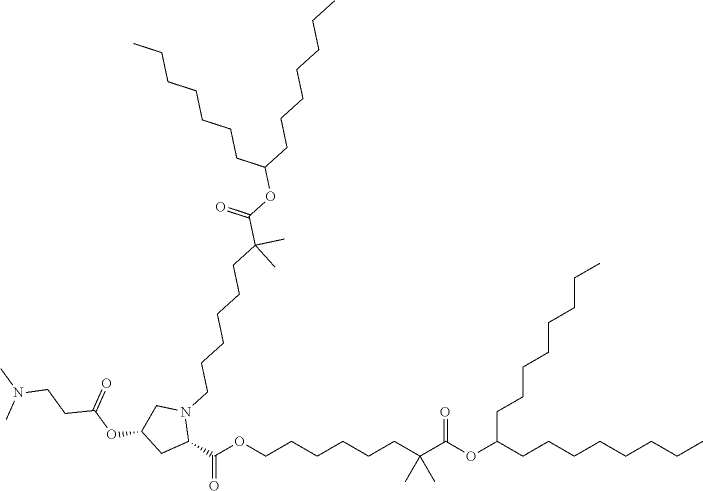 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)OC(CCCCCCC)CCCCCCC] | 8-(heptadecan-9- yloxy)-7,7-dimethyl- 8-oxooctyl (2S,4S)- 1-(7,7-dimethyl-8- oxo-8-(pentadecan- 8-yloxy)octyl)-4-((3- (dimethylamino) propanoyl)oxy) pyrrolidine- 2-carboxylate |
| 2484 | 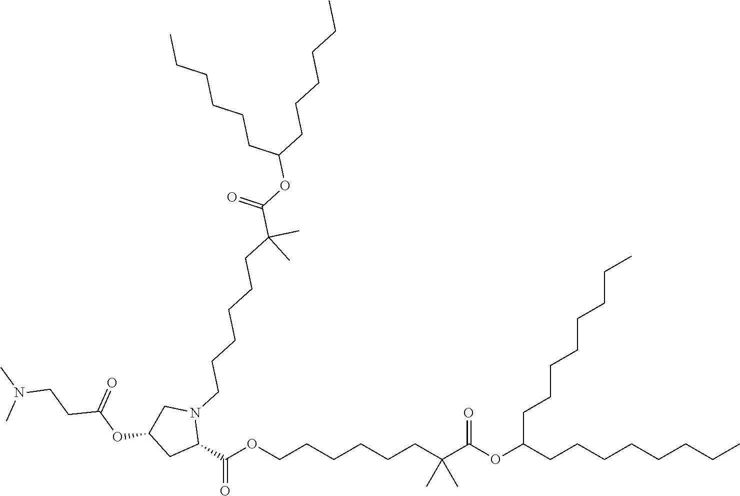 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)OC(CCCCCC)CCCCCC] | 8-(heptadecan-9- yloxy)-7,7-dimethyl- 8-oxooctyl (2S,4S)- 1-(7,7-dimethyl-8- oxo-8-(tridecan-7- yloxy)octyl)-4-((3- (dimethylamino) propanoyl)oxy) pyrrolidine- 2-carboxylate |
| 2482 | 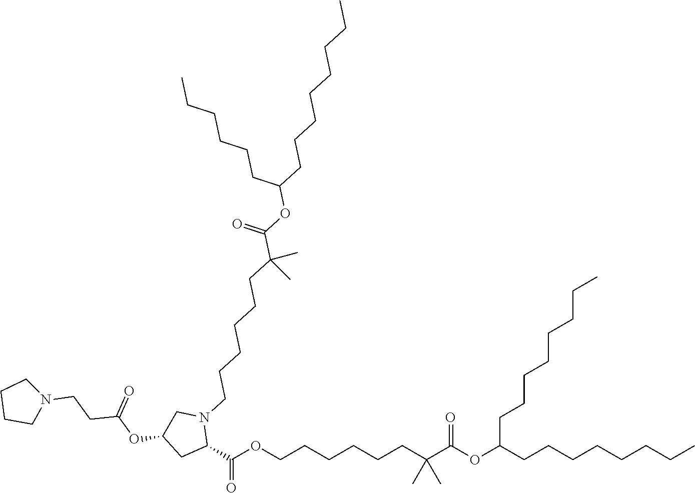 [SMILES: CCCCCCCCC(CCCCCC)OC(=O)C(C)(C)CCCCCCN1C[C@@H](OC(=O)CCN2CCCC2)C[C@H]1C(=O)OCCCCCCC(C)(C)C(=O)OC(CCCCCCCC)CCCCCCCC] | 8-(heptadecan-9- yloxy)-7,7-dimethyl- 8-oxooctyl (2S,4S)- 1-(7,7-dimethyl-8- oxo-8-(pentadecan- 7-yloxy)octyl)-4-((3- (pyrrolidin-1- yl)propanoyl)oxy) pyrrolidine-2- carboxylate |
| 2535 | 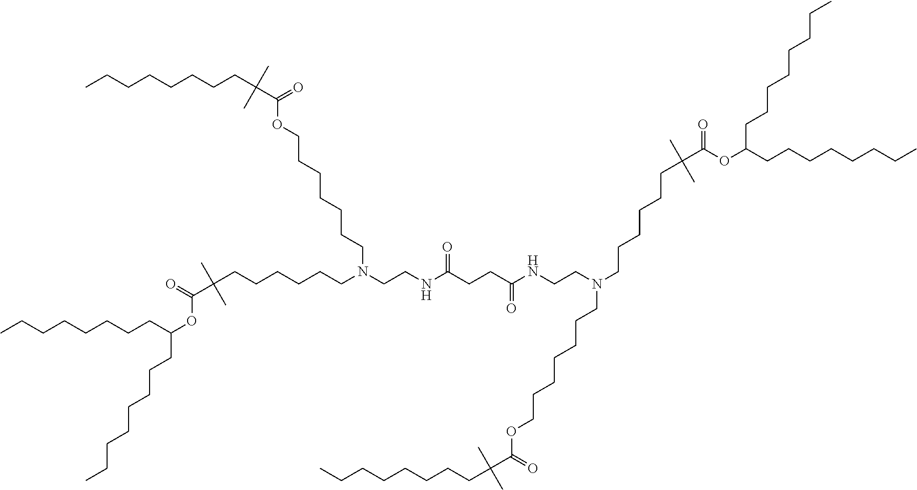 [SMILES: CCCCCCCCC(CCCCCCCC)OC(=O)C(C)(C)CCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCC(C)(C)C(=O)OC(CCCCCCCC)CCCCCCCC] | |
| 2536 | 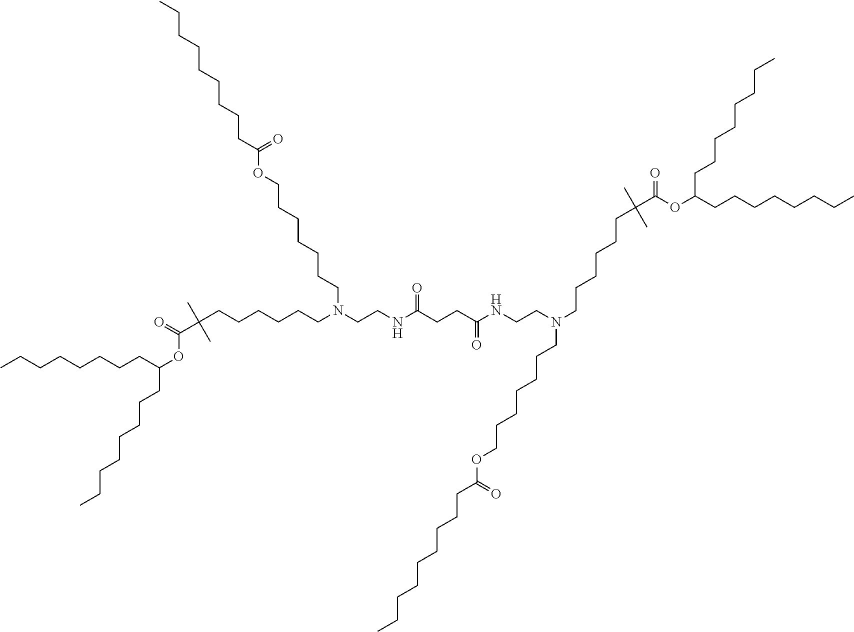 [SMILES: CCCCCCCCCC(=O)OCCCCCCCN(CCCCCCC(C)(C)C(=O)OC(CCCCCCCC)CCCCCCCC)CCNC(=O)CCC(=O)NCCN(CCCCCCCOC(=O)CCCCCCCCC)CCCCCCC(C)(C)C(=O)OC(CCCCCCCC)CCCCCCCC] | |
| 2537 | 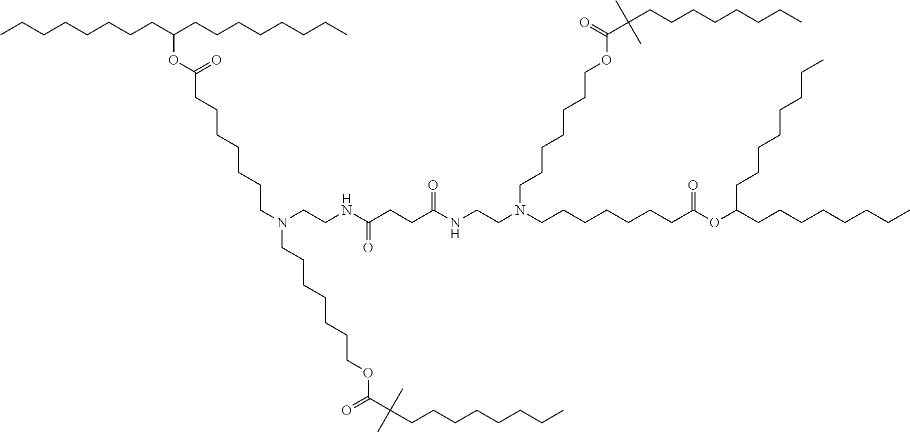 [SMILES: CCCCCCCCC(CCCCCCCC)OC(=O)CCCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCCC(=O)OC(CCCCCCCC)CCCCCCCC] | |
| 2544 | 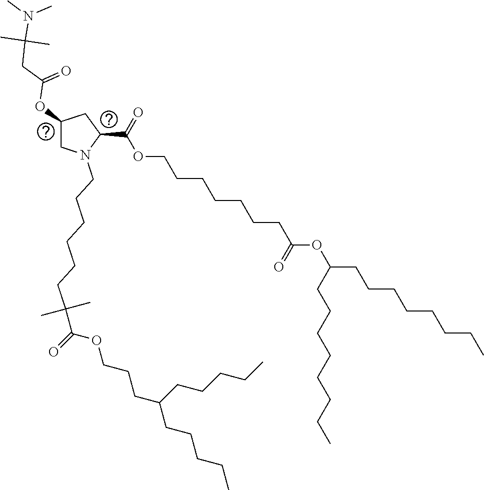 [SMILES: CCCCCCCCC(CCCCCCCC)OC(=O)CCCCCCCOC(=O)[C@@H]1C[C@H](OC(=O)CC(C)(C)N(C)C)CN1CCCCCCC(C)(C)C(=O)OCCCC(CCCCC)CCCCC] [symbol] | |
| 2545 | 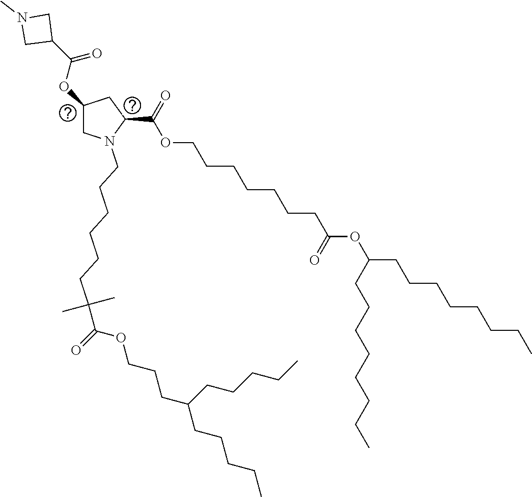 [SMILES: CCCCCCCCC(CCCCCCCC)OC(=O)CCCCCCCOC(=O)[C@@H]1C[C@H](OC(=O)C2CN(C)C2)CN1CCCCCCC(C)(C)C(=O)OCCCC(CCCCC)CCCCC] | |
| 2546 | 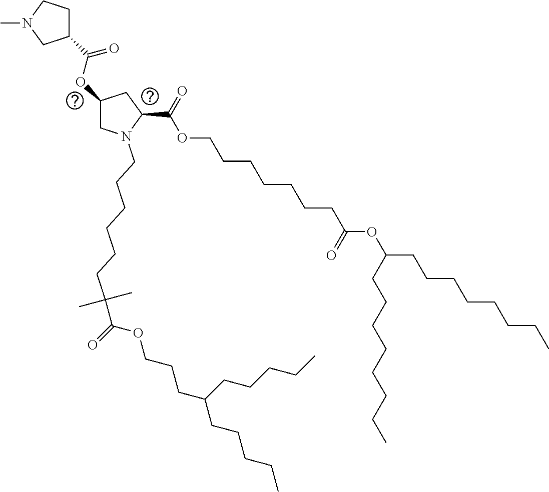 [SMILES: CCCCCCCCC(CCCCCCCC)OC(=O)CCCCCCCOC(=O)[C@@H]1C[C@H](OC(=O)[C@H]2CCN(C)C2)CN1CCCCCCC(C)(C)C(=O)OCCCC(CCCCC)CCCCC] | |
| 2547 | 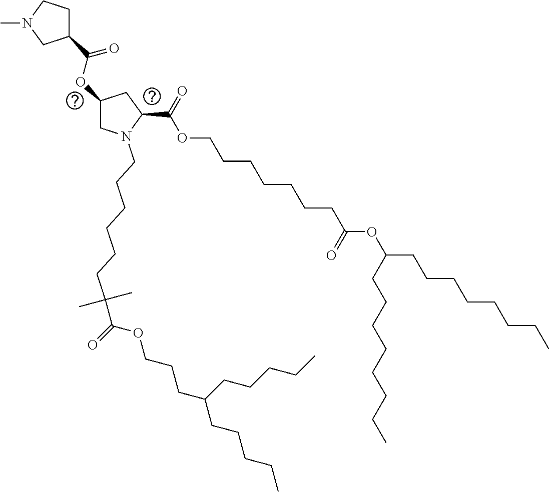 [SMILES: CCCCCCCCC(CCCCCCCC)OC(=O)CCCCCCCOC(=O)[C@@H]1C[C@H](OC(=O)[C@@H]2CCN(C)C2)CN1CCCCCCC(C)(C)C(=O)OCCCC(CCCCC)CCCCC] | |
| 2551 | 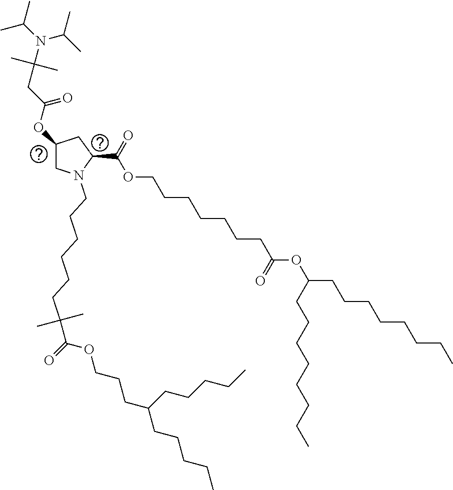 [SMILES: CCCCCCCCC(CCCCCCCC)OC(=O)CCCCCCCOC(=O)[C@@H]1C[C@H](OC(=O)CC(C)(C)N(C(C)C)C(C)C)CN1CCCCCCC(C)(C)C(=O)OCCCC(CCCCC)CCCCC] | |
| 2552 | 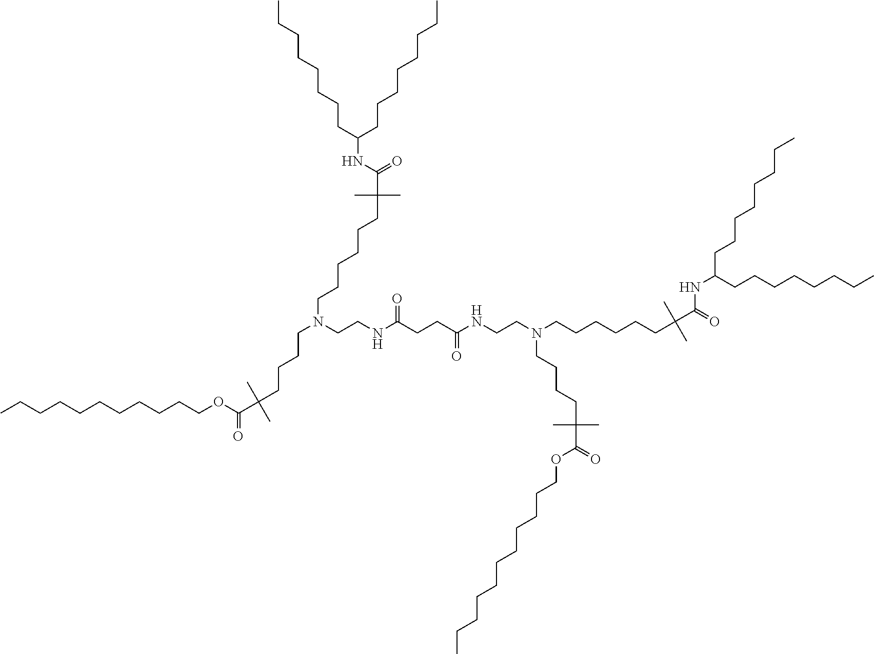 [SMILES: CCCCCCCCCCCOC(=O)C(C)(C)CCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OCCCCCCCCCCC] | |
| 2553 | 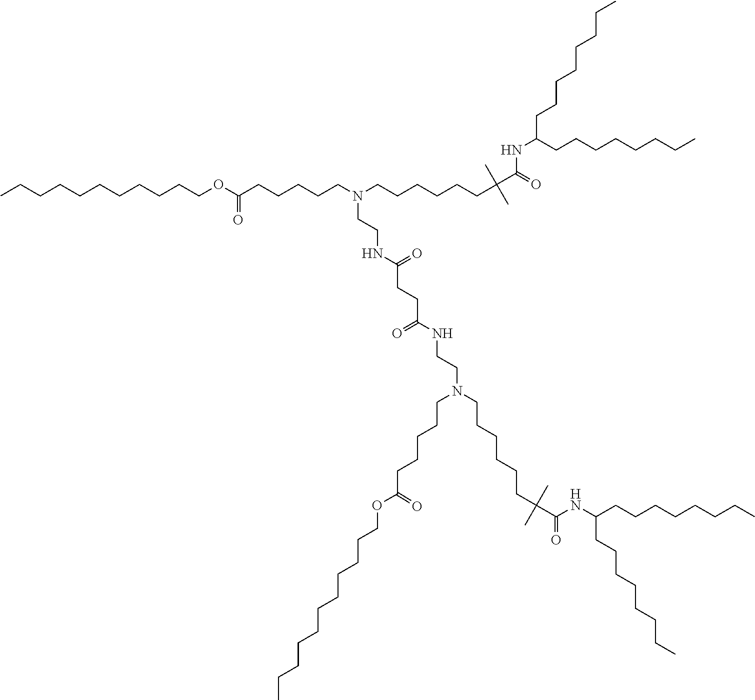 [SMILES: CCCCCCCCCCCOC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)OCCCCCCCCCCC] | |
| 2554 | 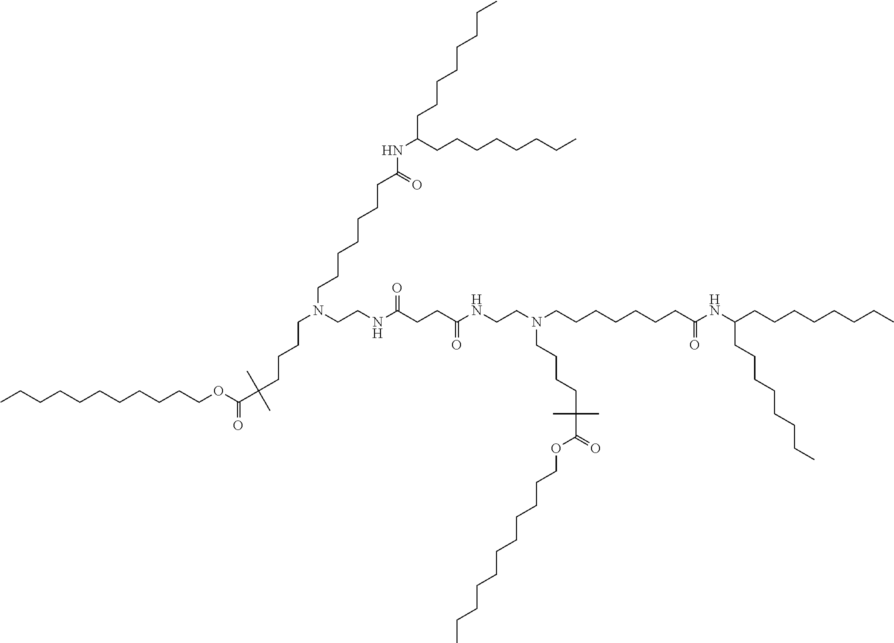 [SMILES: CCCCCCCCCCCOC(=O)C(C)(C)CCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OCCCCCCCCCCC] | |
| 2555 | 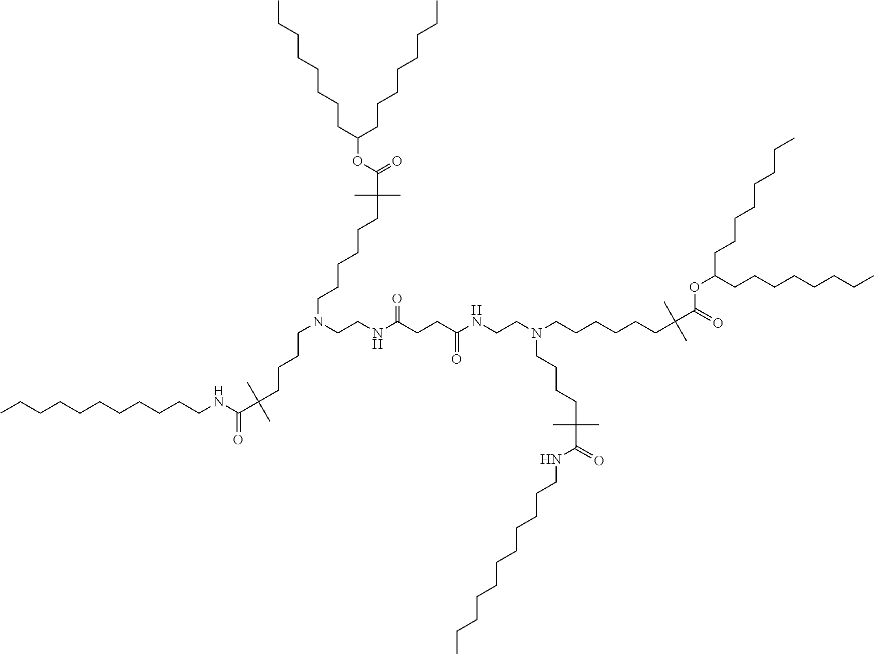 [SMILES: CCCCCCCCCCCNC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)NCCCCCCCCCCC] | |
| 2556 | 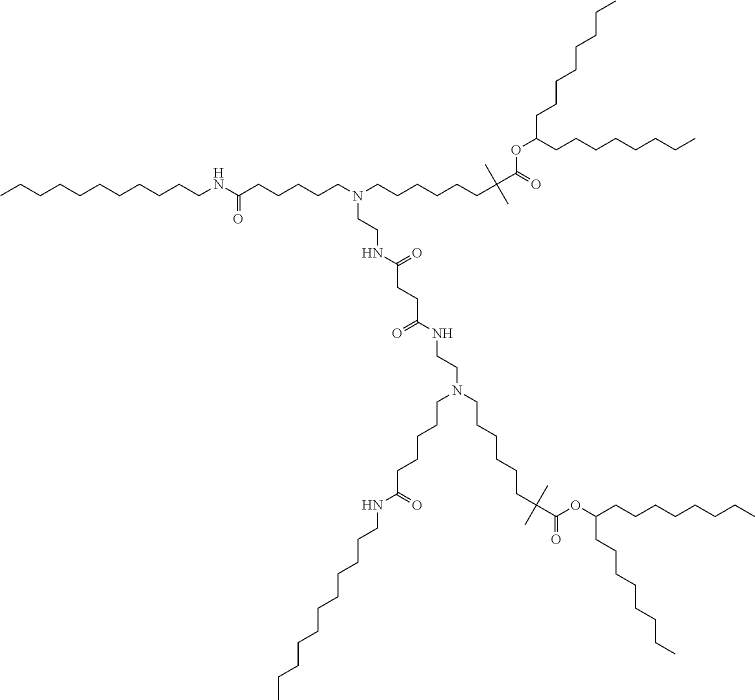 [SMILES: CCCCCCCCCCCNC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)NCCCCCCCCCCC] | |
| 2557 | 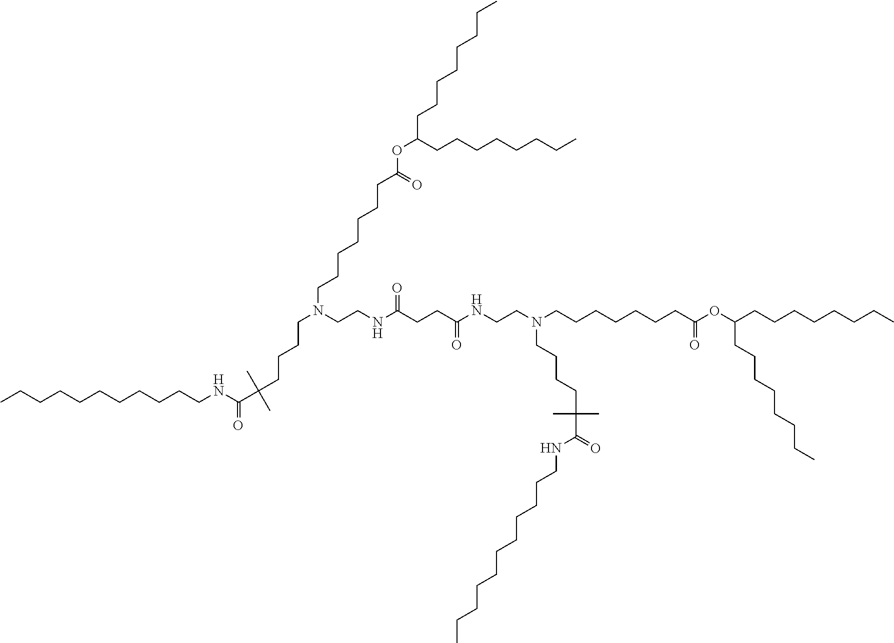 [SMILES: CCCCCCCCCCCNC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)NCCCCCCCCCCC] | |
| 2559 | 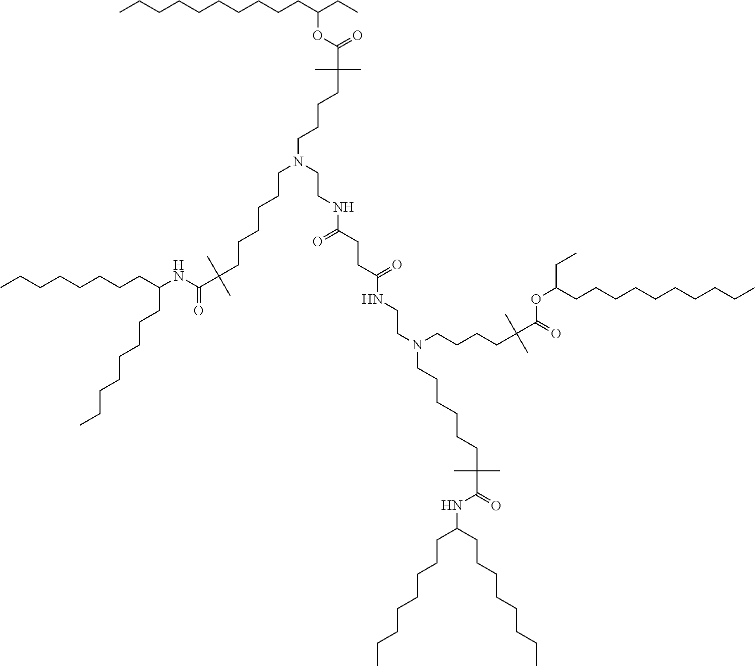 [SMILES: CCCCCCCCCCC(CC)OC(=O)C(C)(C)CCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OC(CC)CCCCCCCCCC] | |
| 2560 | 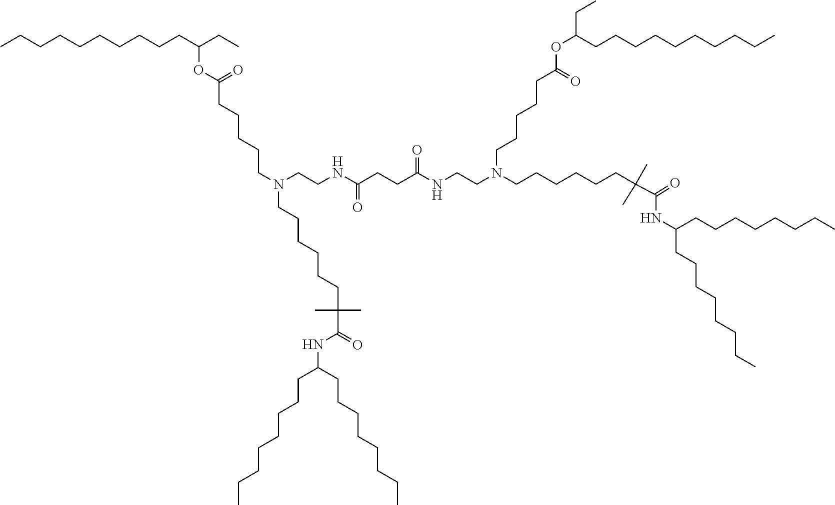 [SMILES: CCCCCCCCCCC(CC)OC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)OC(CC)CCCCCCCCCC] | |
| 2561 | 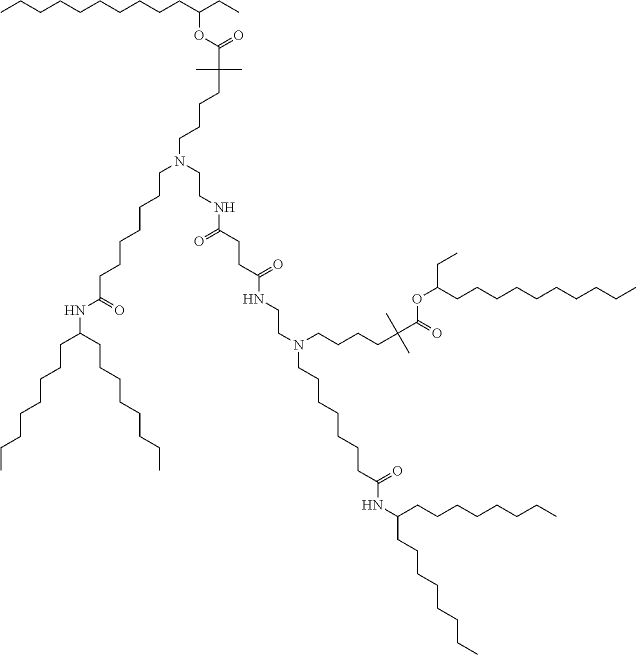 [SMILES: CCCCCCCCCCC(CC)OC(=O)C(C)(C)CCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OC(CC)CCCCCCCCCC] | |
| 2563 | 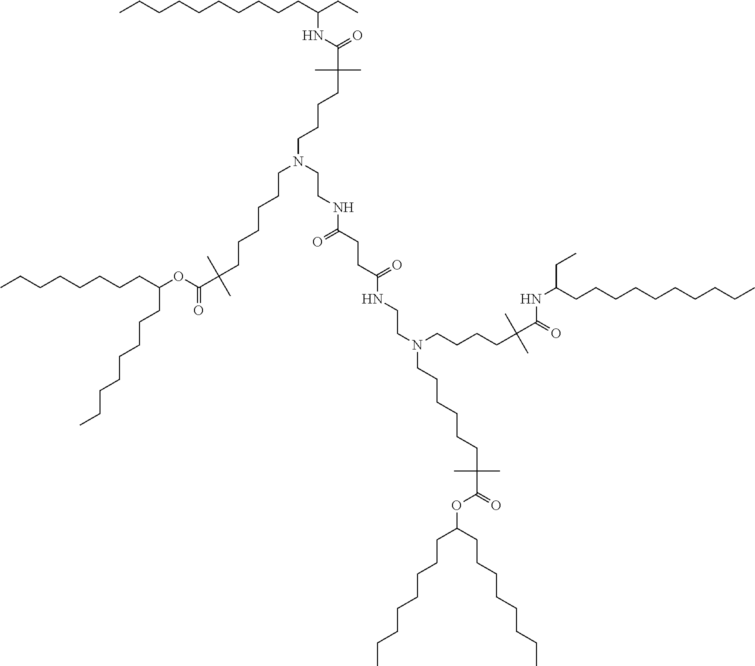 [SMILES: CCCCCCCCCCC(CC)NC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)NC(CC)CCCCCCCCCC] | |
| 2564 | 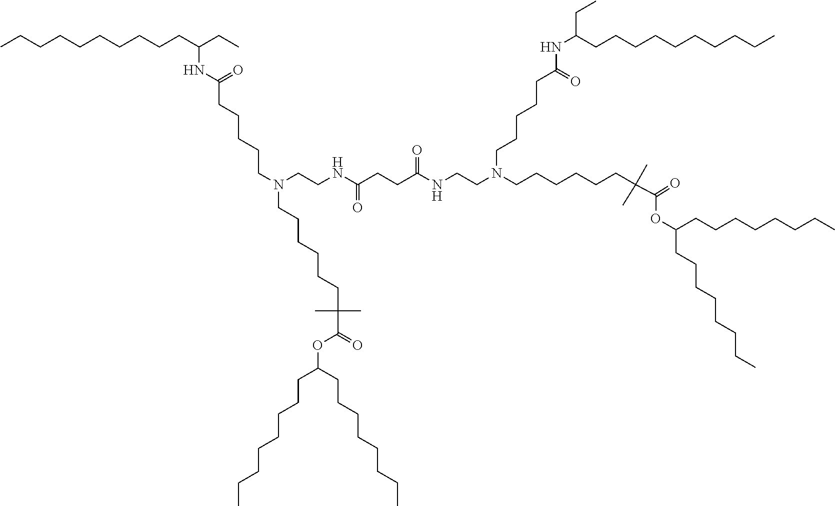 [SMILES: CCCCCCCCCCC(CC)NC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)NC(CC)CCCCCCCCCC] | |
| 2565 | 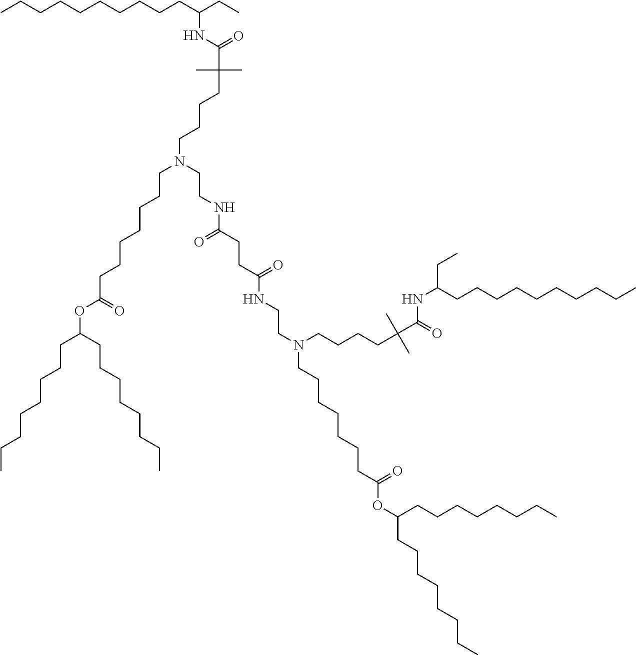 [SMILES: CCCCCCCCCCC(CC)NC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)NC(CC)CCCCCCCCCC] | |
| 2566 | 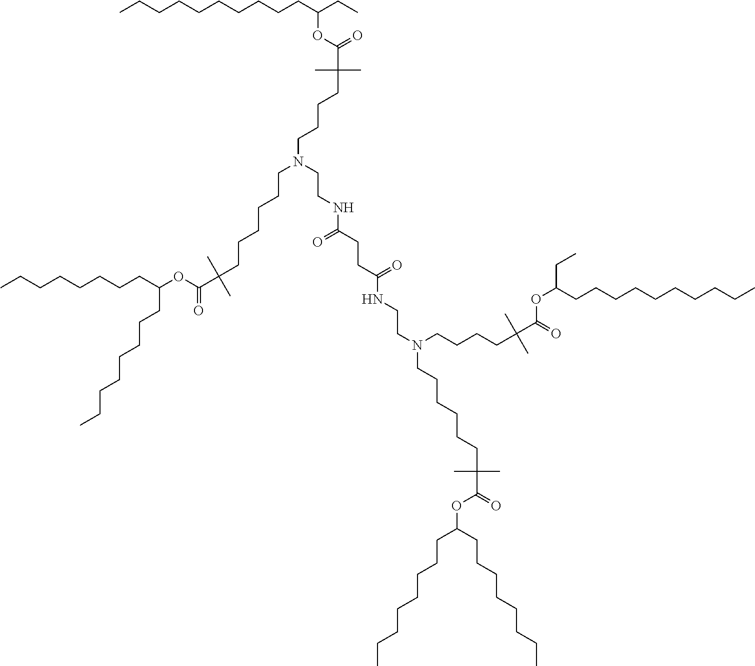 [SMILES: CCCCCCCCCCC(CC)OC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OC(CC)CCCCCCCCCC] | |
| 2567 | 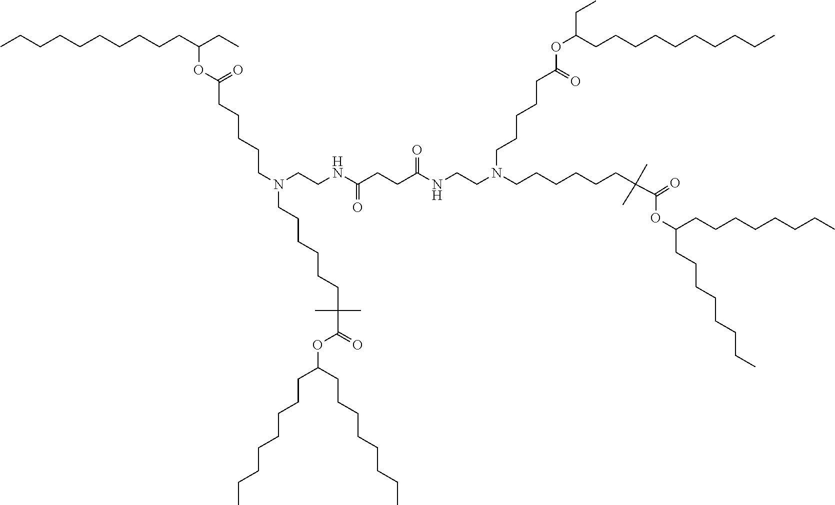 [SMILES: CCCCCCCCCCC(CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)OC(CC)CCCCCCCCCC] | |
| 2568 | 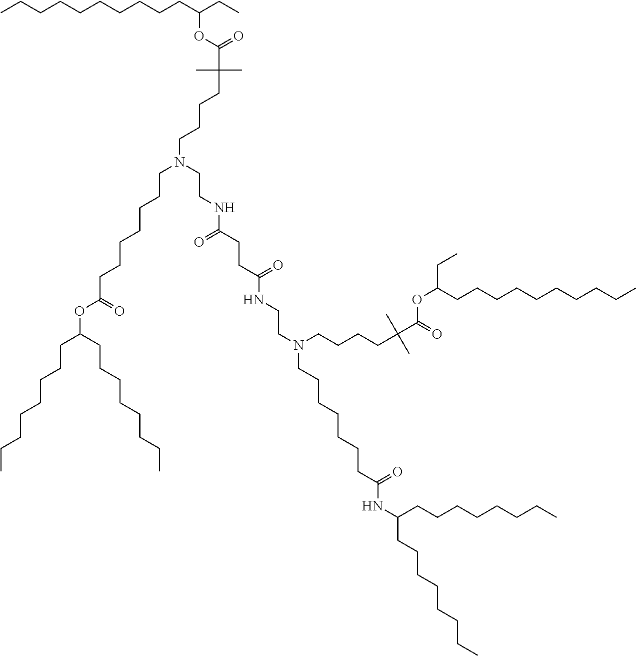 [SMILES: CCCCCCCCCCC(CC)OC(=O)C(C)(C)CCCCN(CCCCCCCC(=O)NC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OC(CC)CCCCCCCCCC] | |
| 2569 | 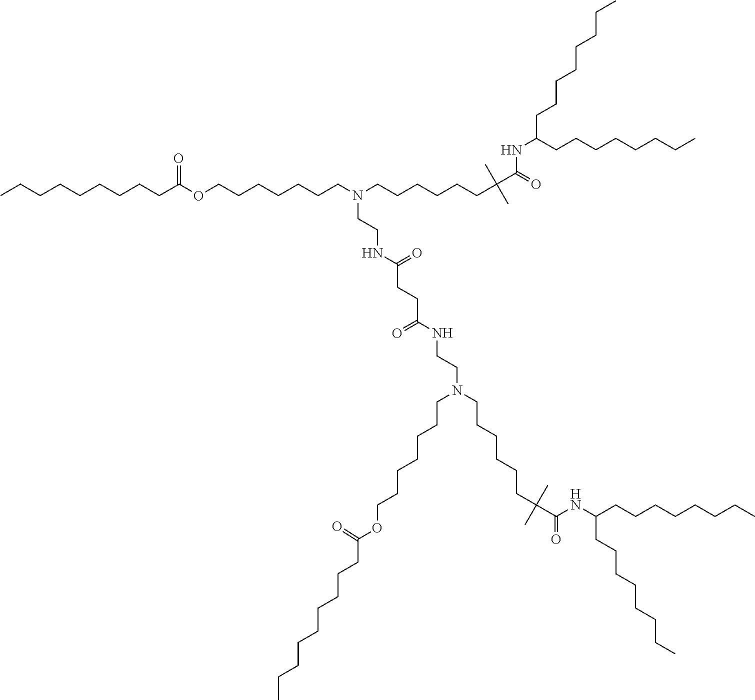 [SMILES: CCCCCCCCCC(=O)OCCCCCCCN(CCCCCCC(C)(C)C(=O)NC(CCCCCCCC)CCCCCCCC)CCNC(=O)CCC(=O)NCCN(CCCCCCCOC(=O)CCCCCCCCC)CCCCCCC(C)(C)C(=O)NC(CCCCCCCC)CCCCCCCC] | |
| 2570 | 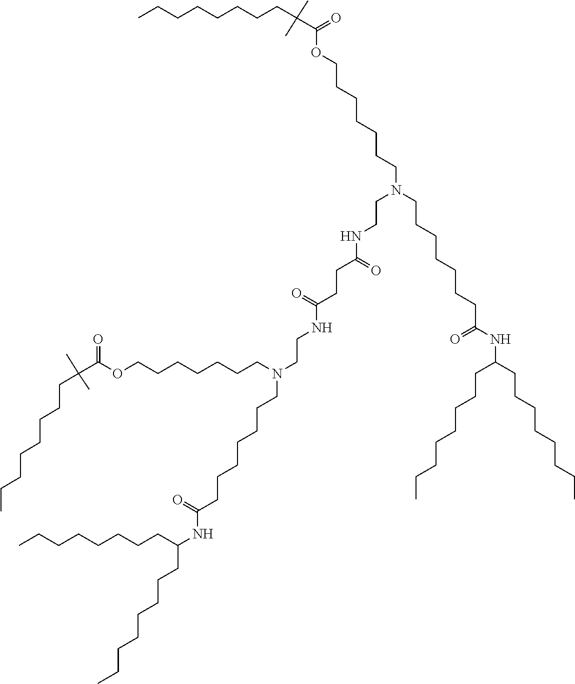 [SMILES: CCCCCCCCC(CCCCCCCC)NC(=O)CCCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCCC(=O)NC(CCCCCCCC)CCCCCCCC] | |
| 2571 | 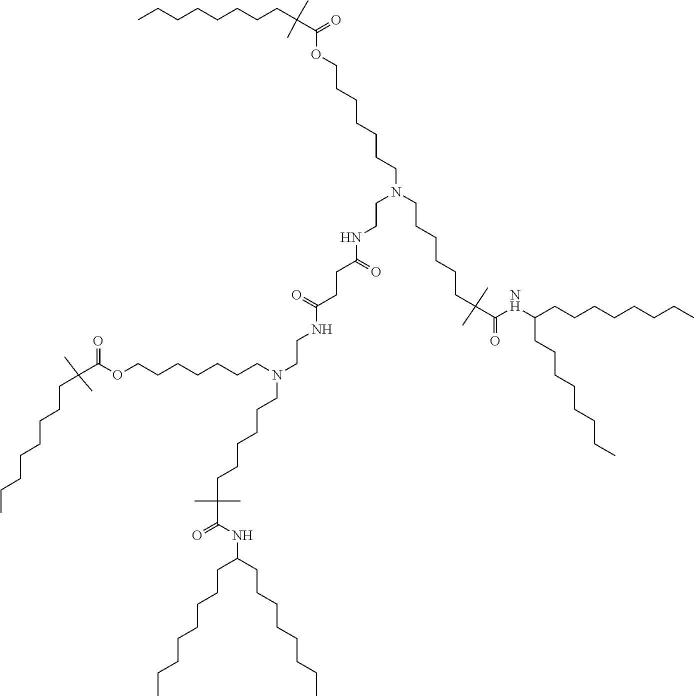 [SMILES: CCCCCCCCC(CCCCCCCC)NC(=O)C(C)(C)CCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCC(C)(C)C(=O)NC(CCCCCCCC)CCCCCCCC] | |
| 2573 | 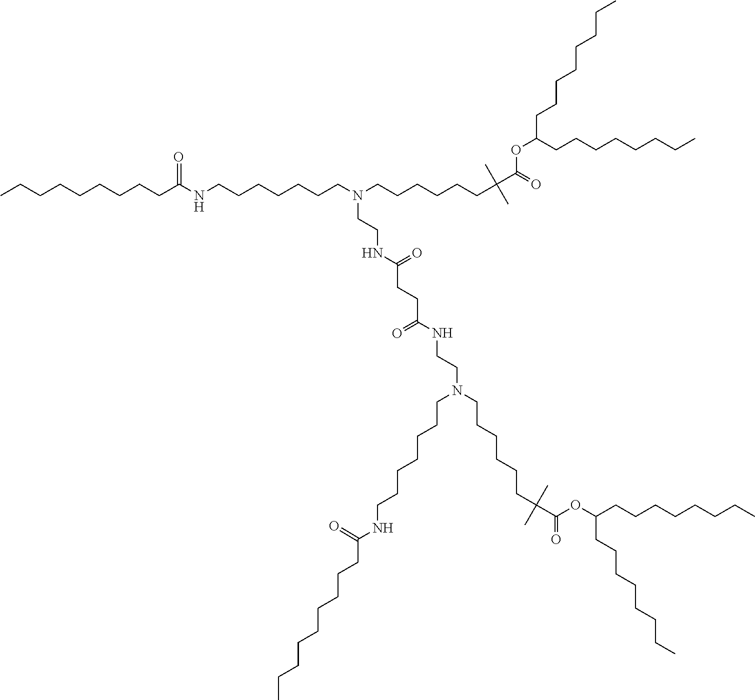 [SMILES: CCCCCCCCCC(=O)NCCCCCCCN(CCCCCCC(C)(C)C(=O)OC(CCCCCCCC)CCCCCCCC)CCNC(=O)CCC(=O)NCCN(CCCCCCCNC(=O)CCCCCCCCC)CCCCCCC(C)(C)C(=O)OC(CCCCCCCC)CCCCCCCC] | |
| 2574 | 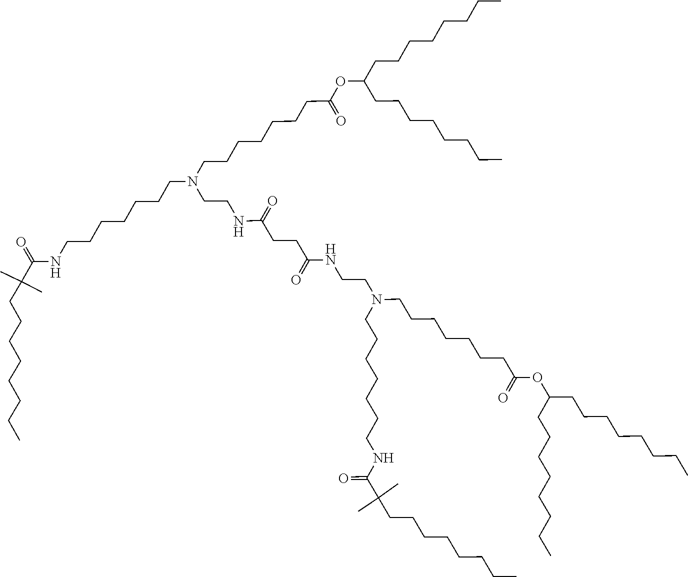 [SMILES: CCCCCCCCC(CCCCCCCC)OC(=O)CCCCCCCN(CCCCCCCNC(=O)C(C)(C)CCCCCCCC)CCNC(=O)CCC(=O)NCCN(CCCCCCCNC(=O)C(C)(C)CCCCCCCC)CCCCCCCC(=O)OC(CCCCCCCC)CCCCCCCC] | |
| 2575 | 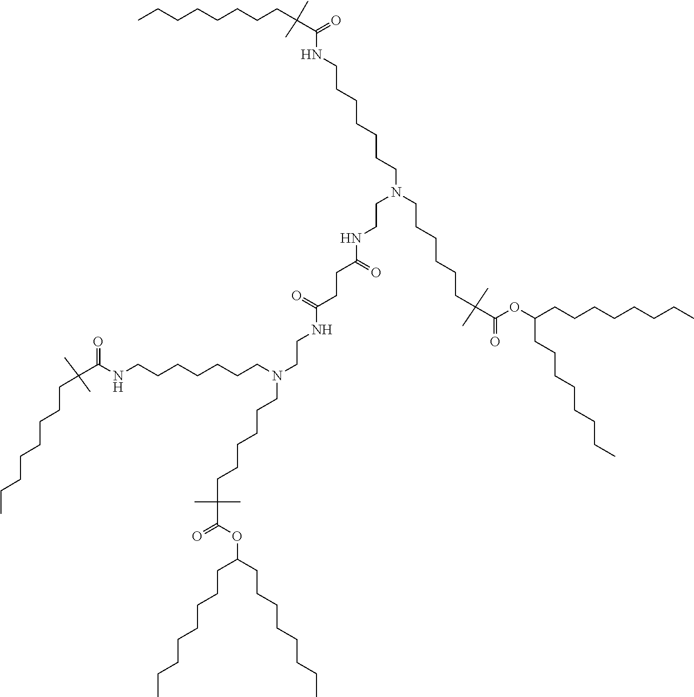 [SMILES: CCCCCCCCC(CCCCCCCC)OC(=O)C(C)(C)CCCCCCN(CCCCCCCNC(=O)C(C)(C)CCCCCCCC)CCNC(=O)CCC(=O)NCCN(CCCCCCCNC(=O)C(C)(C)CCCCCCCC)CCCCCCC(C)(C)C(=O)OC(CCCCCCCC)CCCCCCCC] | |
| 2577 | 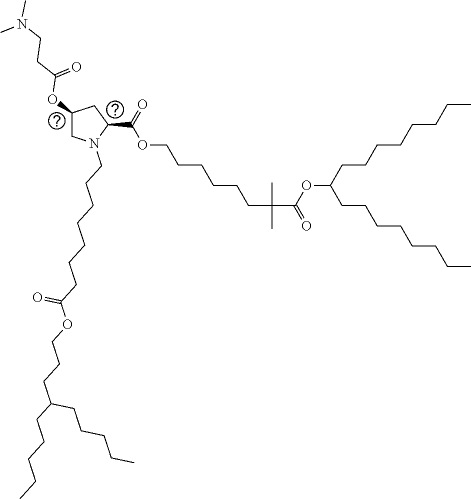 [SMILES: CCCCCCCCC(CCCCCCCC)OC(=O)C(C)(C)CCCCCCOC(=O)[C@@H]1C[C@H](OC(=O)CCN(C)C)CN1CCCCCCCC(=O)OCCCC(CCCCC)CCCCC] | |
| 2578 | 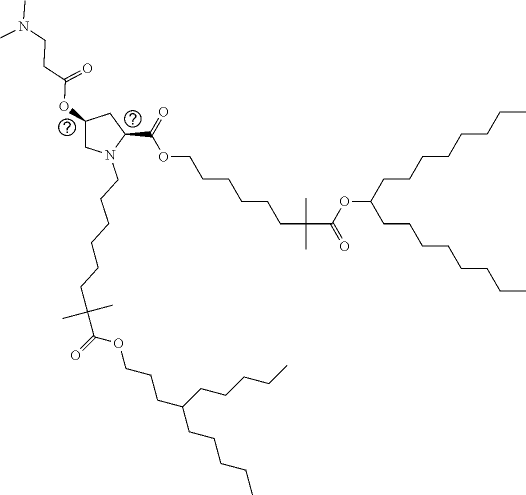 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)OCCCC(CCCCC)CCCCC] | |
| 2580 | 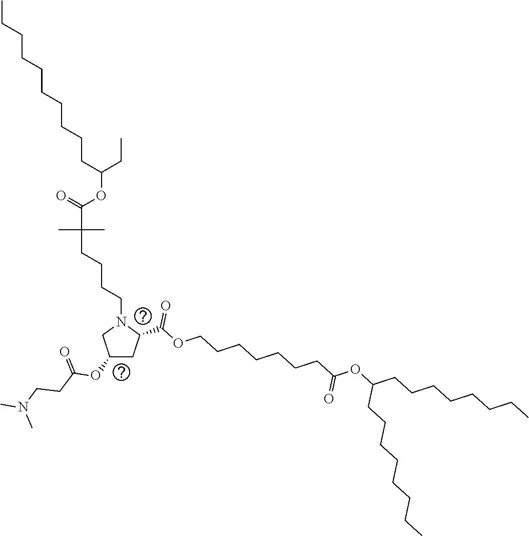 [SMILES: CCCCCCCCCCC(CC)OC(=O)C(C)(C)CCCCN1C[C@@H](OC(=O)CCN(C)C)C[C@H]1C(=O)OCCCCCCCC(=O)OC(CCCCCCCC)CCCCCCCC] | |
| 2581 | 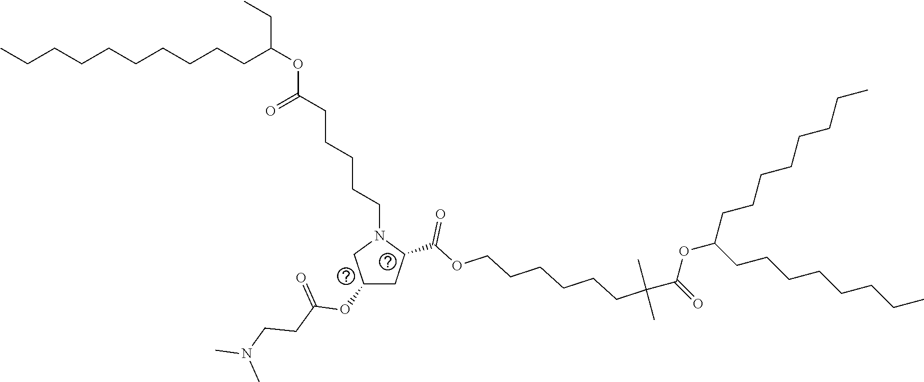 [SMILES: CCCCCCCCCCC(CC)OC(=O)CCCCCN1C[C@@H](OC(=O)CCN(C)C)C[C@H]1C(=O)OCCCCCCC(C)(C)C(=O)OC(CCCCCCCC)CCCCCCCC] | |
| 2582 | 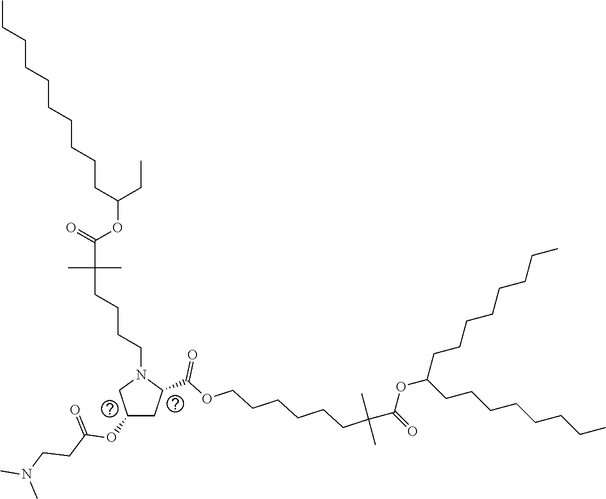 [SMILES: CCCCCCCCCCC(CC)OC(=O)C(C)(C)CCCCN1C[C@@H](OC(=O)CCN(C)C)C[C@H]1C(=O)OCCCCCCC(C)(C)C(=O)OC(CCCCCCCC)CCCCCCCC] | |
| 2583 | 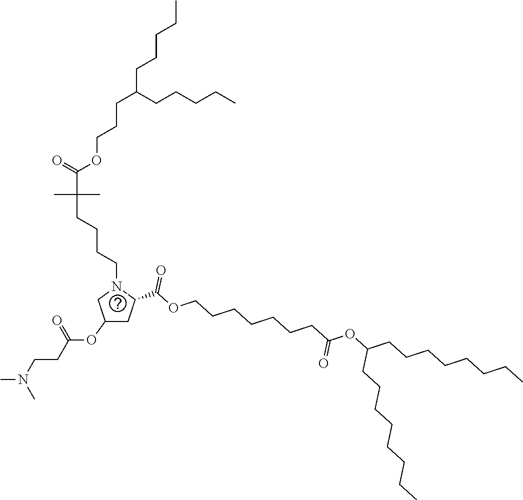 [SMILES: CCCCCCCCC(CCCCCCCC)OC(=O)CCCCCCCOC(=O)[C@@H]1CC(OC(=O)CCN(C)C)CN1CCCCC(C)(C)C(=O)OCCCC(CCCCC)CCCCC] | |
| 2584 | 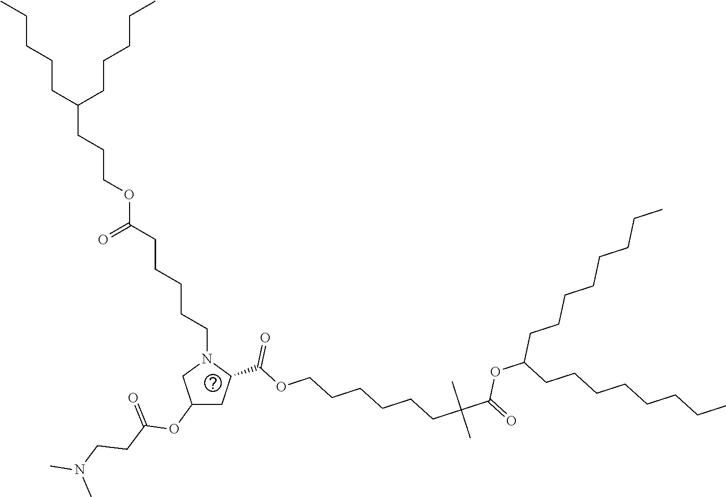 [SMILES: CCCCCCCCC(CCCCCCCC)OC(=O)C(C)(C)CCCCCCOC(=O)[C@@H]1CC(OC(=O)CCN(C)C)CN1CCCCCC(=O)OCCCC(CCCCC)CCCCC] | |
| 2585 | 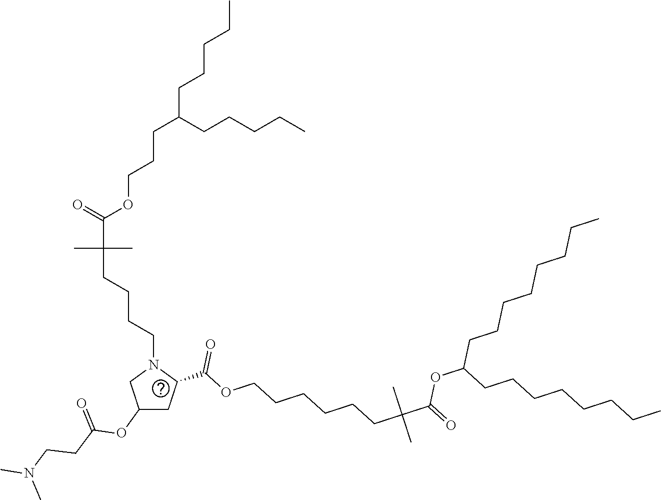 [SMILES: CCCCCCCCC(CCCCCCCC)OC(=O)C(C)(C)CCCCCCOC(=O)[C@@H]1CC(OC(=O)CCN(C)C)CN1CCCCC(C)(C)C(=O)OCCCC(CCCCC)CCCCC] | |
| 2586 | 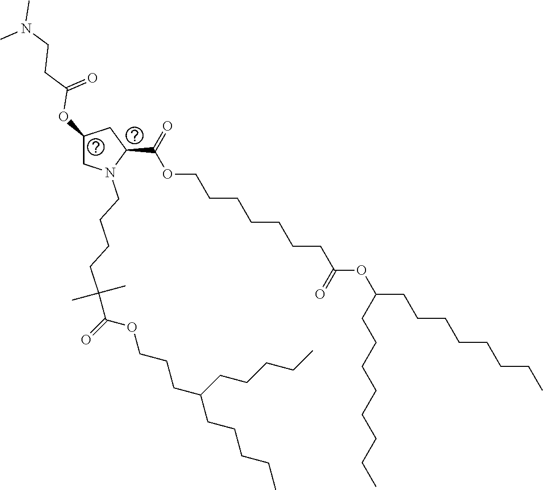 [SMILES: CCCCCCCCC(CCCCCCCC)OC(=O)CCCCCCCOC(=O)[C@@H]1C[C@H](OC(=O)CCN(C)C)CN1CCCCC(C)(C)C(=O)OCCCC(CCCCC)CCCCC] | |
| 2587 | 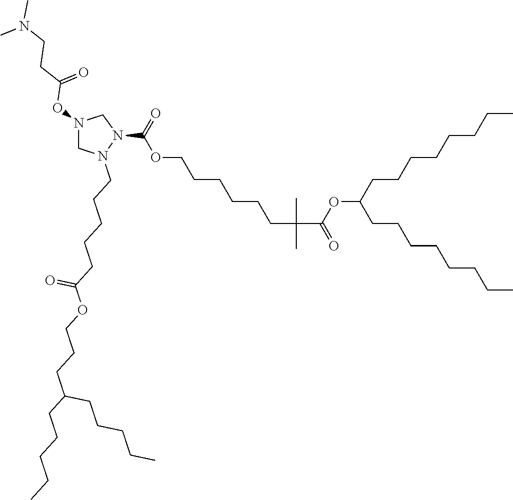 | |
| 2588 | 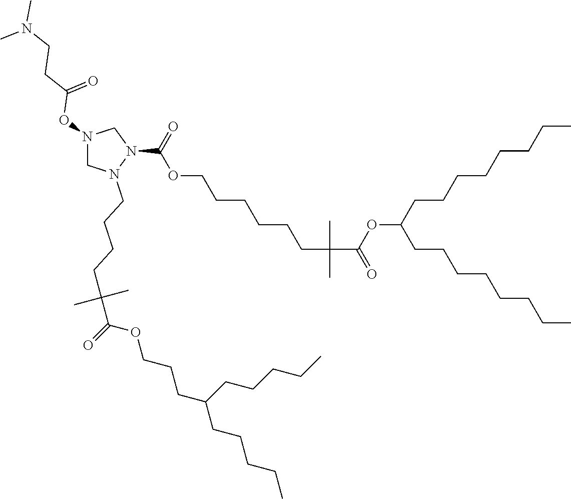 | |
| 2589 | 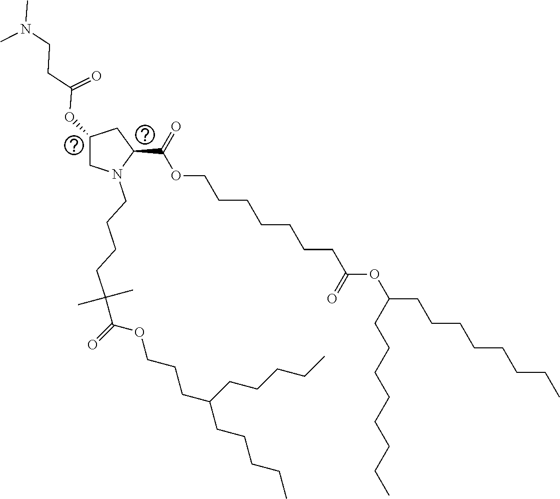 [SMILES: CCCCCCCCC(CCCCCCCC)OC(=O)CCCCCCCOC(=O)[C@@H]1C[C@@H](OC(=O)CCN(C)C)CN1CCCCC(C)(C)C(=O)OCCCC(CCCCC)CCCCC] | |
| 2590 | 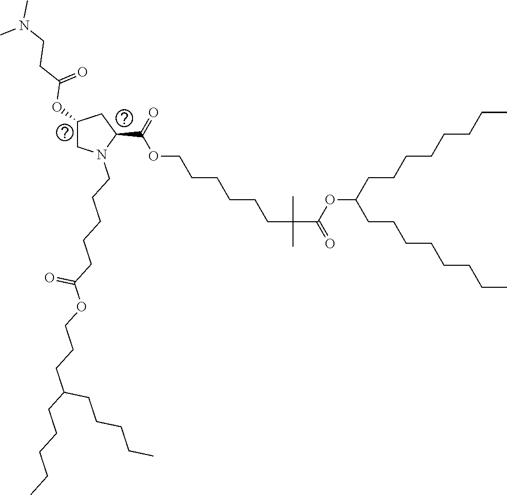 [SMILES: CCCCCCCCC(CCCCCCCC)OC(=O)C(C)(C)CCCCCCOC(=O)[C@@H]1C[C@@H](OC(=O)CCN(C)C)CN1CCCCCC(=O)OCCCC(CCCCC)CCCCC] | |
| 2591 | 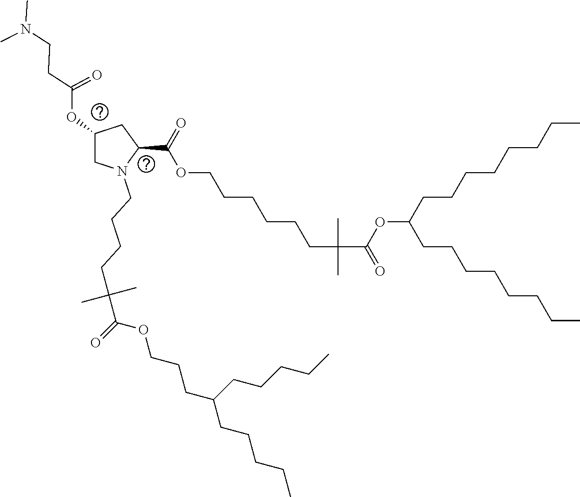 [SMILES: CCCCCCCCC(CCCCCCCC)OC(=O)C(C)(C)CCCCCCOC(=O)[C@@H]1C[C@@H](OC(=O)CCN(C)C)CN1CCCCC(C)(C)C(=O)OCCCC(CCCCC)CCCCC] | |
| 2592 | 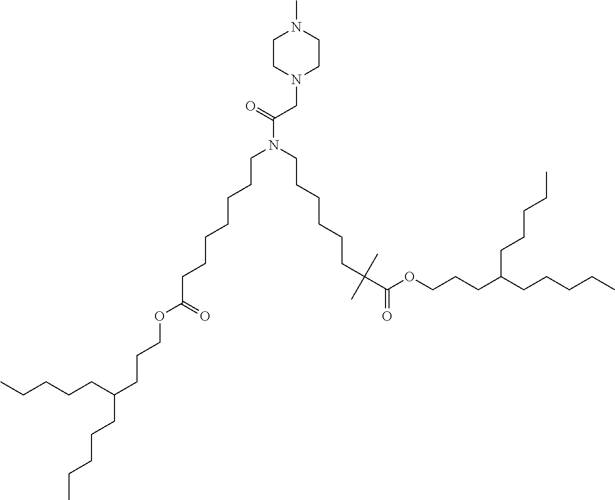 [SMILES: CCCCCC(CCCCC)CCCOC(=O)CCCCCCCN(CCCCCCC(C)(C)C(=O)OCCCC(CCCCC)CCCCC)C(=O)CN1CCN(C)CC1] | |
| 2599 | 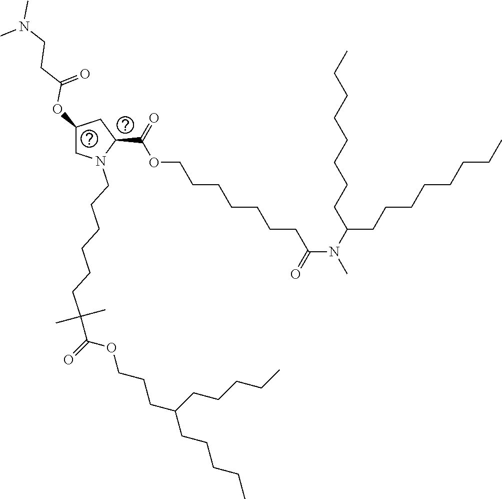 [SMILES: CCCCCCCCC(CCCCCCCC)N(C)C(=O)CCCCCCCOC(=O)[C@@H]1C[C@H](OC(=O)CCN(C)C)CN1CCCCCCC(C)(C)C(=O)OCCCC(CCCCC)CCCCC] | |
| 2600 | 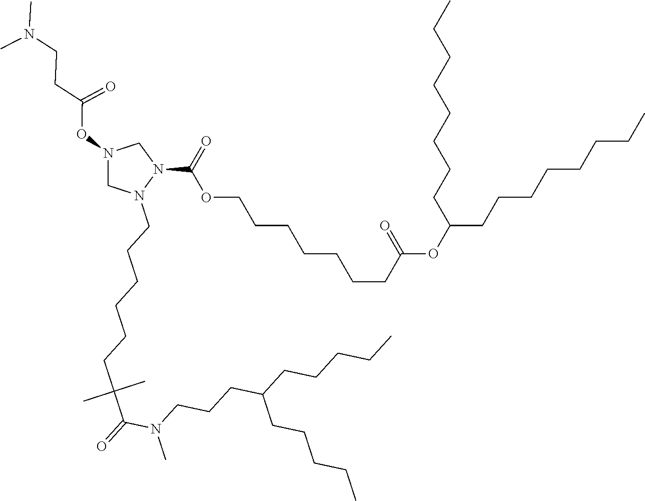 | |
| 2601 | 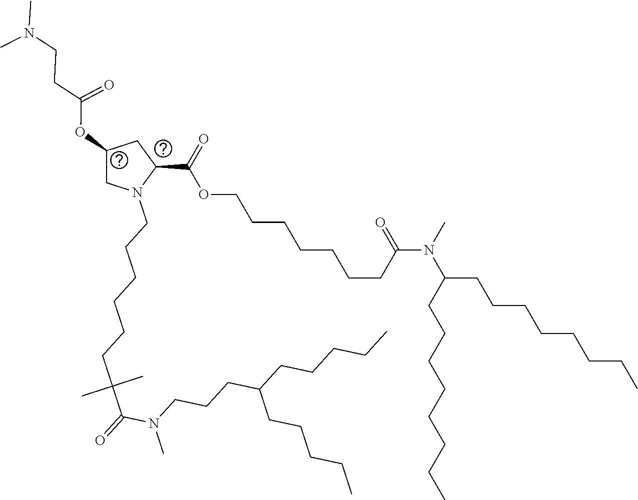 [SMILES: CCCCCCCCC(CCCCCCCC)N(C)C(=O)CCCCCCCOC(=O)[C@@H]1C[C@H](OC(=O)CCN(C)C)CN1CCCCCCC(C)(C)C(=O)N(C)CCCC(CCCCC)CCCCC] | |
| 2608 | 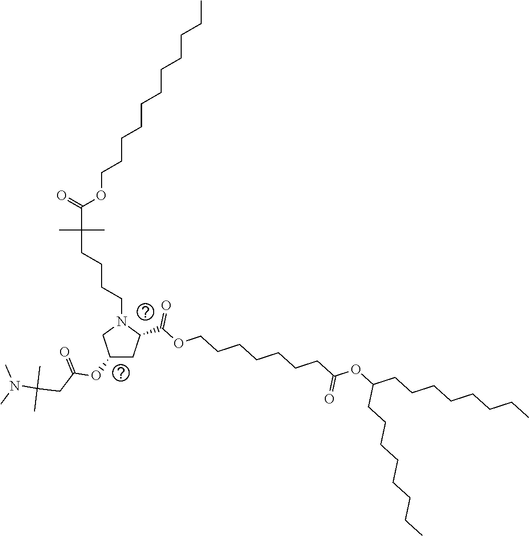 [SMILES: CCCCCCCCCCCOC(=O)C(C)(C)CCCCN1C[C@@H](OC(=O)CC(C)(C)N(C)C)C[C@H]1C(=O)OCCCCCCCC(=O)OC(CCCCCCCC)CCCCCCCC] | |
| 2609 | 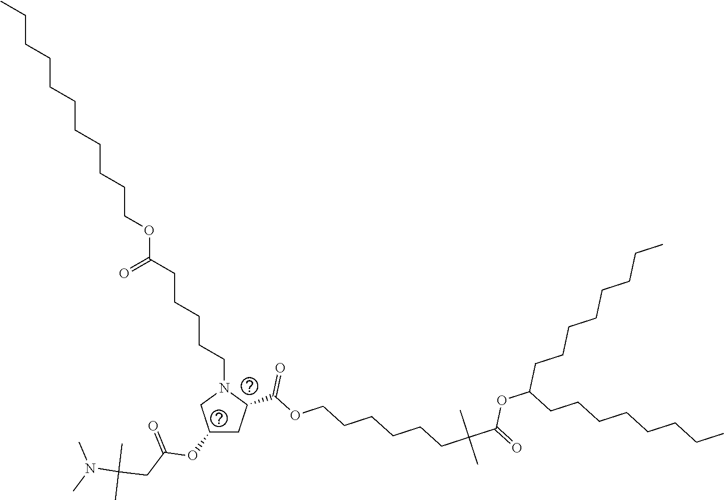 [SMILES: CCCCCCCCCCCOC(=O)CCCCCN1C[C@@H](OC(=O)CC(C)(C)N(C)C)C[C@H]1C(=O)OCCCCCCC(C)(C)C(=O)OC(CCCCCCCC)CCCCCCCC] | |
| 2610 | 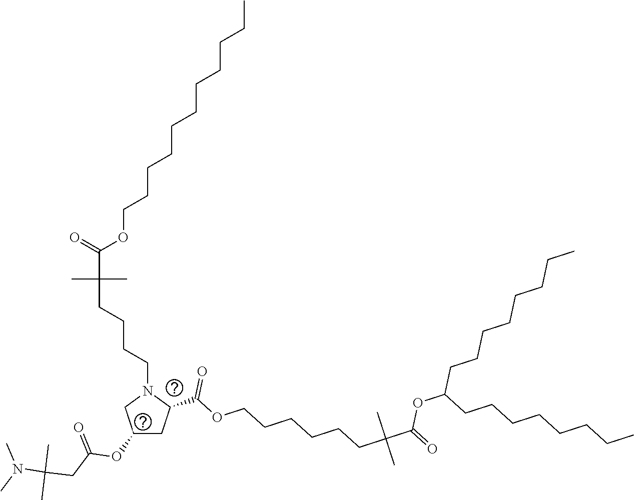 [SMILES: CCCCCCCCCCCOC(=O)C(C)(C)CCCCN1C[C@@H](OC(=O)CC(C)(C)N(C)C)C[C@H]1C(=O)OCCCCCCC(C)(C)C(=O)OC(CCCCCCCC)CCCCCCCC] | |
| 2611 | 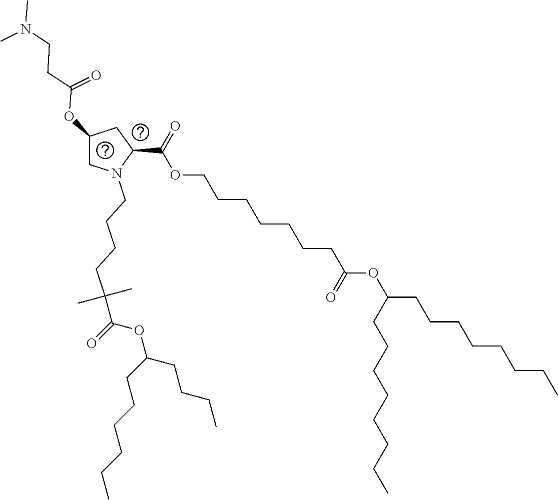 [SMILES: CCCCCCCCC(CCCCCCCC)OC(=O)CCCCCCCOC(=O)[C@@H]1C[C@H](OC(=O)CCN(C)C)CN1CCCCC(C)(C)C(=O)OC(CCCC)CCCCCC] | |
| 2612 | 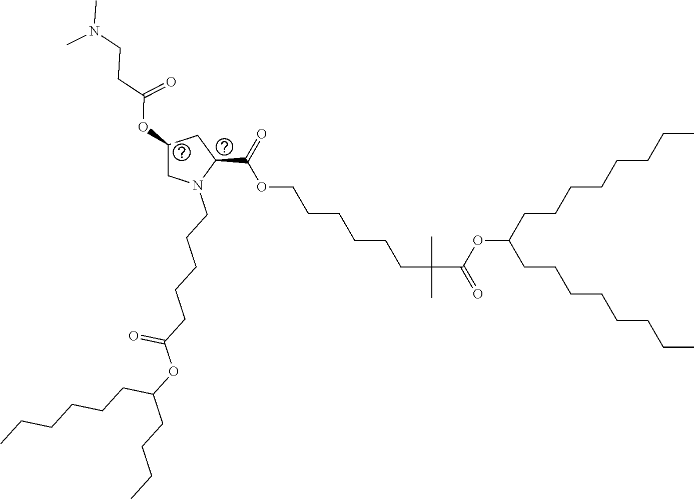 [SMILES: CCCCCCCCC(CCCCCCCC)OC(=O)C(C)(C)CCCCCCOC(=O)[C@@H]1C[C@H](OC(=O)CCN(C)C)CN1CCCCCC(=O)OC(CCCC)CCCCCC] | |
| 2613 | 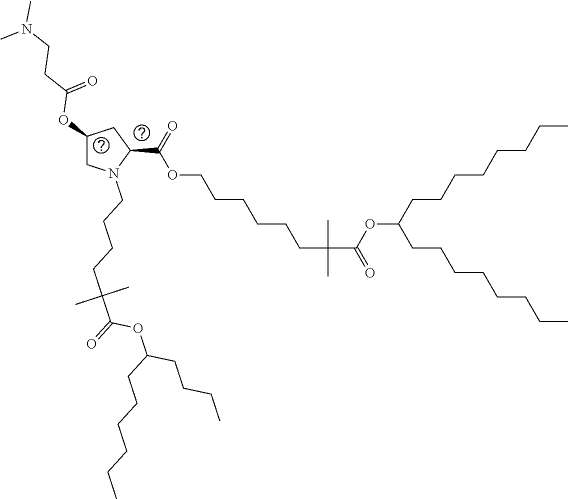 [SMILES: CCCCCCCCC(CCCCCCCC)OC(=O)C(C)(C)CCCCCCOC(=O)[C@@H]1C[C@H](OC(=O)CCN(C)C)CN1CCCCC(C)(C)C(=O)OC(CCCC)CCCCCC] | |
| A1 | 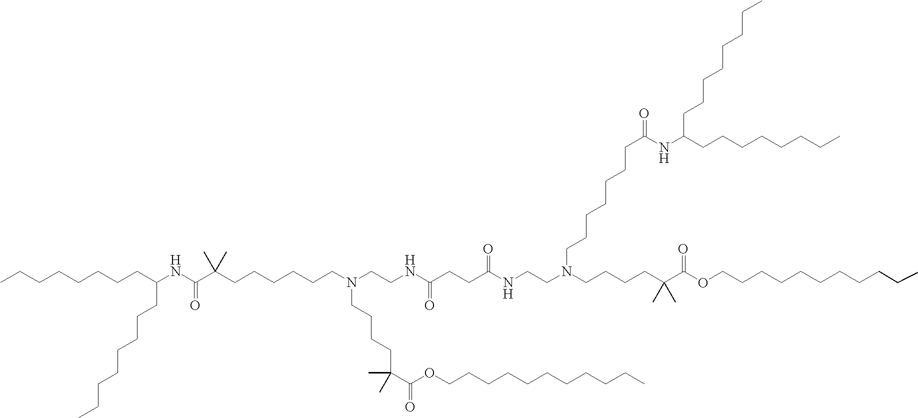 [SMILES: CCCCCCCCCCCOC(=O)C(C)(C)CCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OCCCCCCCCCCC] | diundecyl 7-(8- (heptadecan-9- ylamino)-7,7- dimethyl-8- oxooctyl)-18-(8- (heptadecan-9- ylamino)-8- oxooctyl)-2,2,23,23- tetramethyl-11,14- dioxo-7,10,15,18- tetraazatetra- cosanedioate |
| A2 | 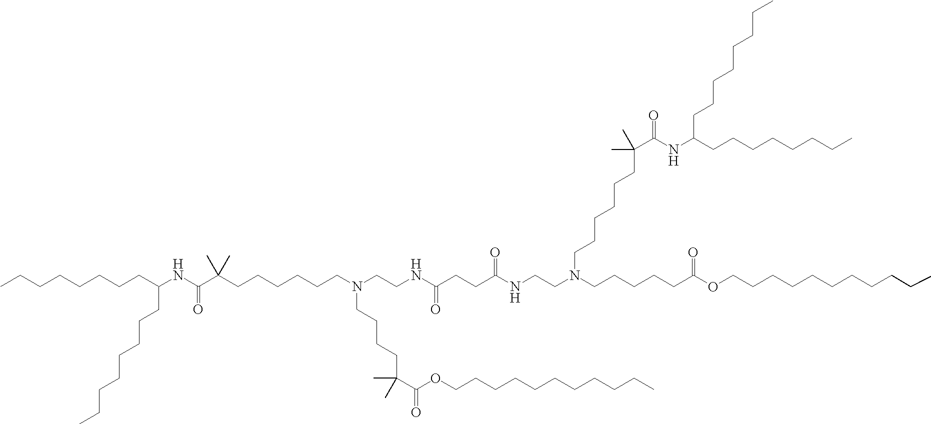 [SMILES: CCCCCCCCCCCOC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OCCCCCCCCCCC] | diundecyl 7,18- bis(8-(heptadecan- 9-ylamino)-7,7- dimethyl-8- oxooctyl)-2,2- dimethyl-11,14- dioxo-7,10,15,18- tetraazatetra- cosanedioate |
| A3 | 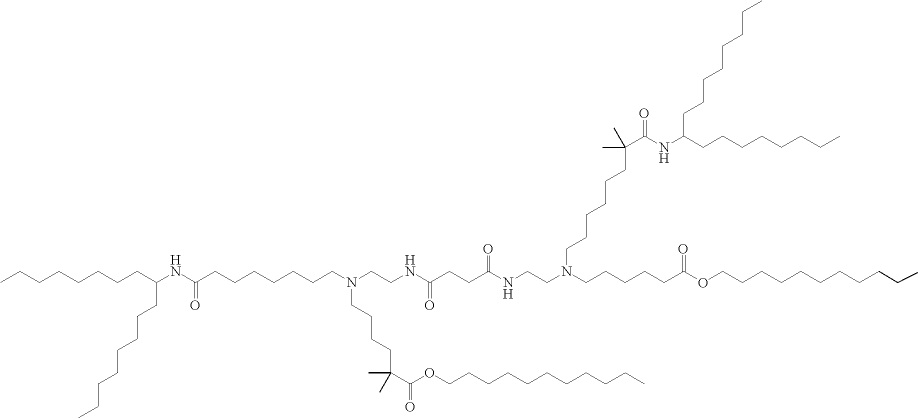 [SMILES: CCCCCCCCCCCOC(=O)CCCCCN(CCCCCCC(C)(C)C(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OCCCCCCCCCCC] | diundecyl 18-(8- (heptadecan-9- ylamino)-7,7- dimethyl-8- oxooctyl)-7-(8- (heptadecan-9- ylamino)-8- oxooctyl)-2,2- dimethyl-11,14- dioxo-7,10,15,18- tetraazatetra- cosanedioate |
| A4 | 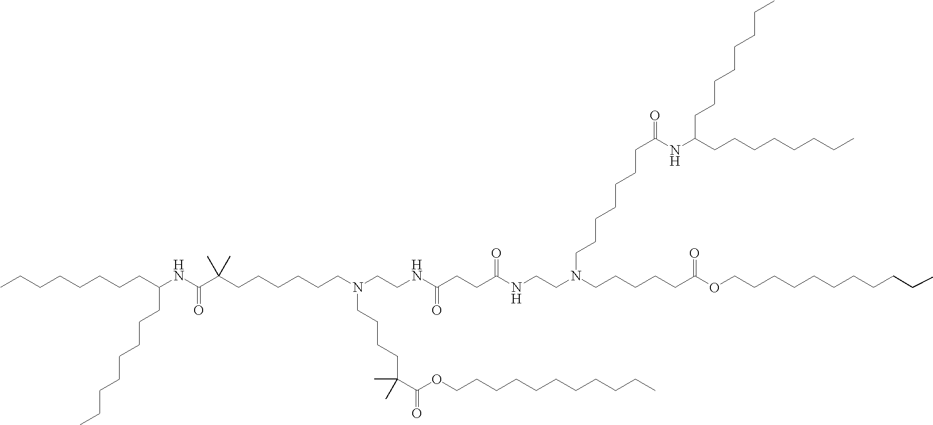 [SMILES: CCCCCCCCCCCOC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OCCCCCCCCCCC] | diundecyl 7-(8- (heptadecan-9- ylamino)-7,7- dimethyl-8- oxooctyl)-18-(8- (heptadecan-9- ylamino)-8- oxooctyl)-2,2- dimethyl-11,14- dioxo-7,10,15,18- tetraazatetra- cosanedioate |
| A5 | 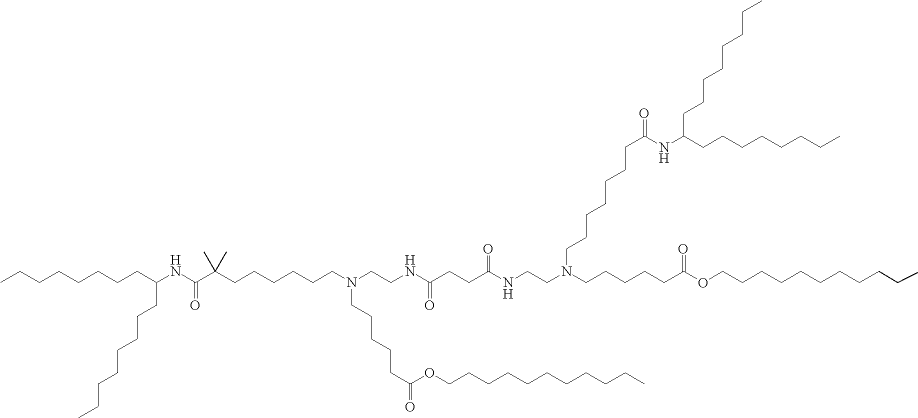 [SMILES: CCCCCCCCCCCOC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)OCCCCCCCCCCC] | diundecyl 7-(8- (heptadecan-9- ylamino)-7,7- dimethyl-8- oxooctyl)-18-(8- (heptadecan-9- ylamino)-8- oxooctyl)-11,14- dioxo-7,10,15,18- tetraazatetra- cosanedioate |
| A6 | 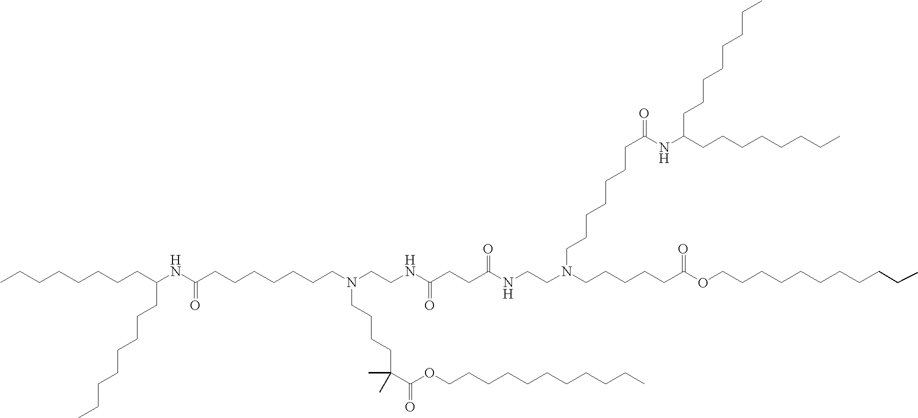 [SMILES: CCCCCCCCCCCOC(=O)CCCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OCCCCCCCCCCC] | diundecyl 7,18- bis(8-(heptadecan- 9-ylamino)-8- oxooctyl)-2,2- dimethyl-11,14- dioxo-7,10,15,18- tetraazatetra- cosanedioate |
| A7 | 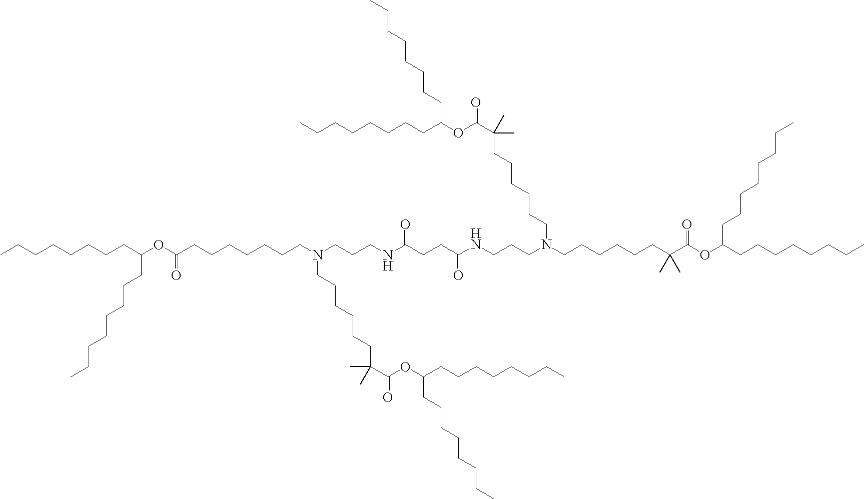 [SMILES: CCCCCCCCC(CCCCCCCC)OC(=O)CCCCCCCN(CCCCCCC(C)(C)C(=O)OC(CCCCCCCC)CCCCCCCC)CCCNC(=O)CCC(=O)NCCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9-(8-(heptadecan-9- yloxy)-7,7-dimethyl- 8-oxooctyl)-22-(8- (heptadecan-9- yloxy)-8-oxooctyl)- 2,2,29,29- tetramethyl-14,17- dioxo-9,13,18,22- tetraaza- triacontanedioate |
| A8 | 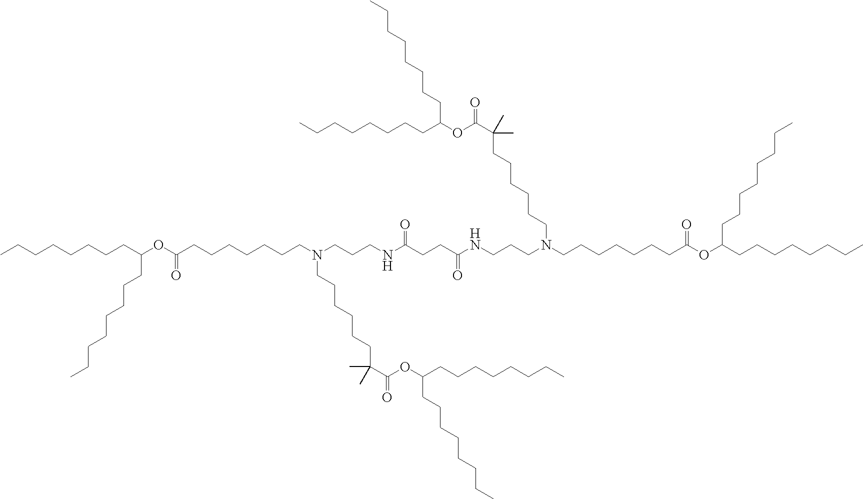 [SMILES: CCCCCCCCC(CCCCCCCC)OC(=O)CCCCCCCN(CCCCCCC(C)(C)C(=O)OC(CCCCCCCC)CCCCCCCC)CCCNC(=O)CCC(=O)NCCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,22-bis(8- (heptadecan-9- yloxy)-8-oxooctyl)- 2,2,29,29- tetramethyl-14,17- dioxo-9,13,18,22- tetraaza- triacontanedioate |
| A9 | 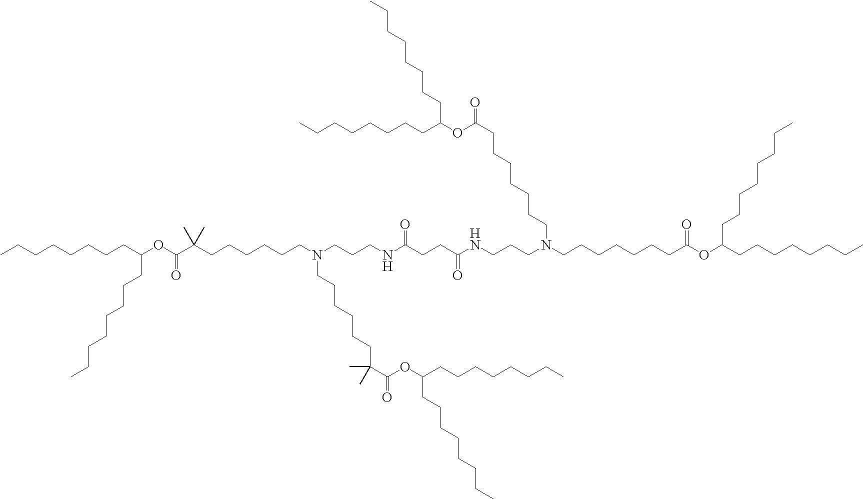 [SMILES: CCCCCCCCC(CCCCCCCC)OC(=O)CCCCCCCN(CCCCCCCC(=O)OC(CCCCCCCC)CCCCCCCC)CCCNC(=O)CCC(=O)NCCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9-(8-(heptadecan-9- yloxy)-7,7-dimethyl- 8-oxooctyl)-22-(8- (heptadecan-9- yloxy)-8-oxooctyl)- 2,2-dimethyl-14,17- dioxo-9,13,18,22- tetraaza- triacontanedioate |
| A10 | 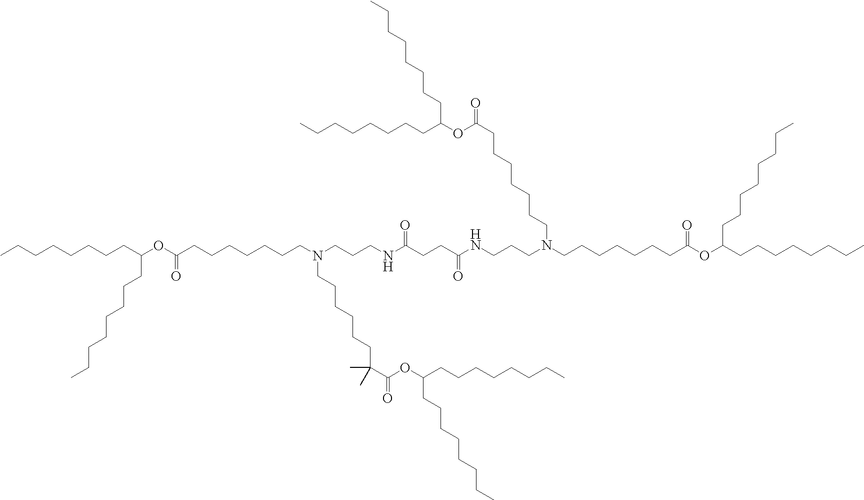 [SMILES: CCCCCCCCC(CCCCCCCC)OC(=O)CCCCCCCN(CCCCCCCC(=O)OC(CCCCCCCC)CCCCCCCC)CCCNC(=O)CCC(=O)NCCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,22-bis(8- (heptadecan-9- yloxy)-8-oxooctyl)- 2,2-dimethyl-14,17- dioxo-9,13,18,22- tetraaza- triacontanedioate |
| A11 | 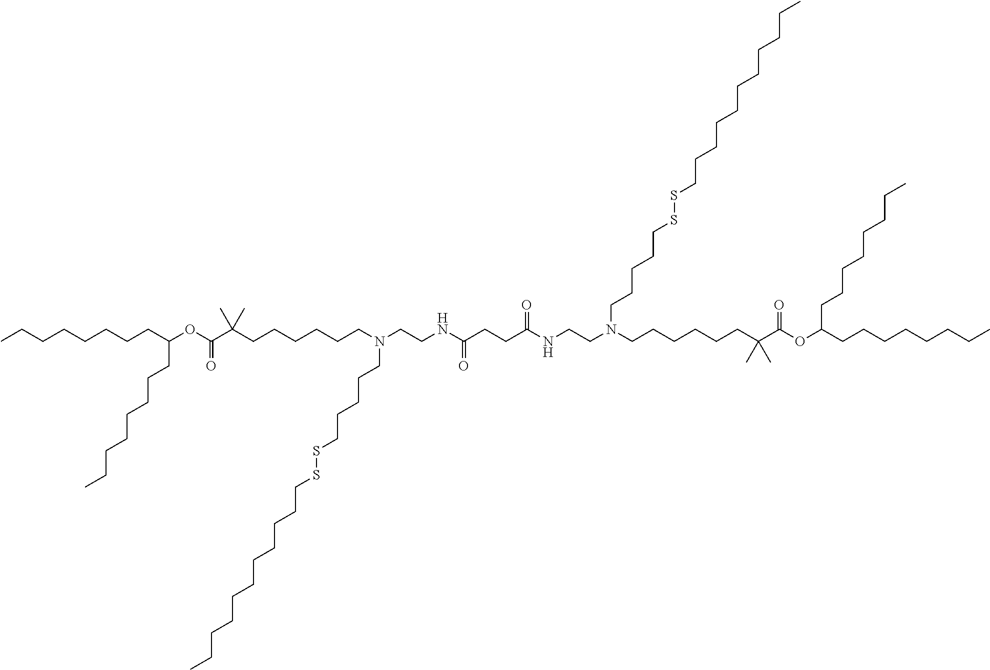 [SMILES: CCCCCCCCCCCSSCCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCSSCCCCCCCCCCC] | di(heptadecan-9-yl) 2,2,27,27- tetramethyl-13,16- dioxo-9,20-bis(5- (undecyldisulfaneyl) pentyl)-9,12,17,20- tetraaza- octacosanedioate |
| A12 | 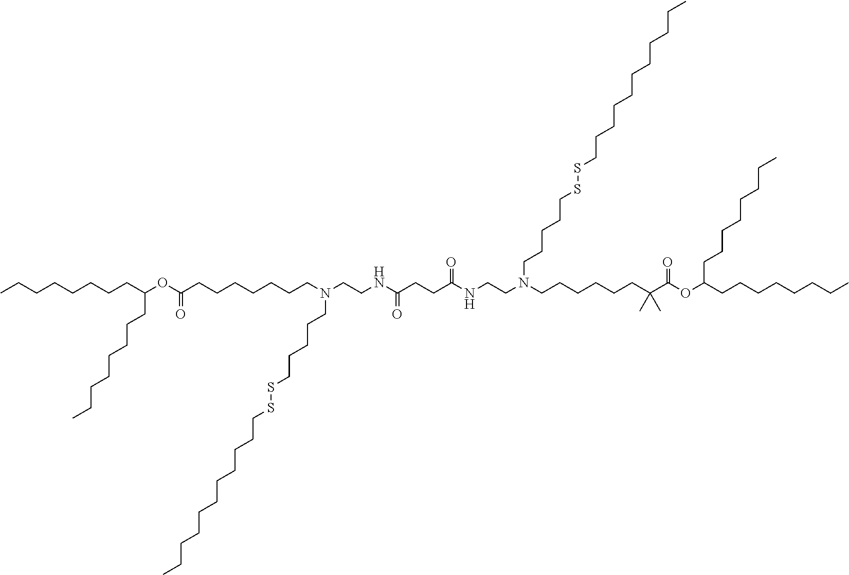 [SMILES: CCCCCCCCCCCSSCCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCSSCCCCCCCCCCC] | di(heptadecan-9-yl) 2,2-dimethyl-13,16- dioxo-9,20-bis(5- (undecyldisulfaneyl) pentyl)-9,12,17,20- tetraaza- octacosanedioate |
| A13 | 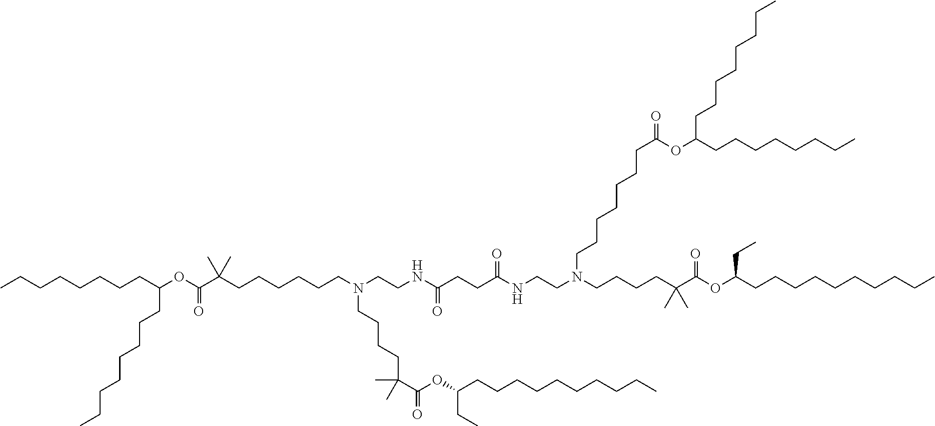 [SMILES: CCCCCCCCCC[C@H](CC)OC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@@H](CC)CCCCCCCCCC] | di(heptadecan-9-yl) 9,20-bis(5,5- dimethyl-6-oxo-6- (((S)-tridecan-3- yl)oxy)hexyl)-2,2- dimethyl-13,16- dioxo-9,12,17,20- tetraaza- octacosanedioate |
| A14 | 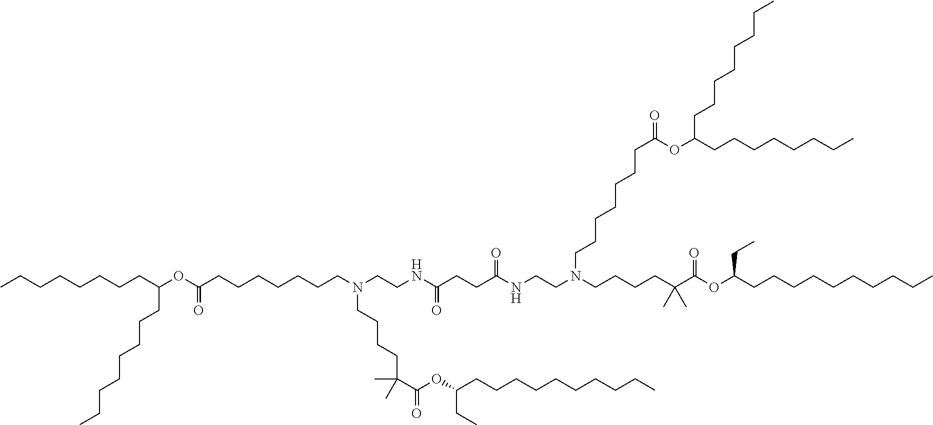 [SMILES: CCCCCCCCCC[C@H](CC)OC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@@H](CC)CCCCCCCCCC] | di(heptadecan-9-yl) 9,20-bis(5,5- dimethyl-6-oxo-6- (((S)-tridecan-3- yl)oxy)hexyl)-13,16- dioxo-9,12,17,20- tetraaza- octacosanedioate |
| A15 | 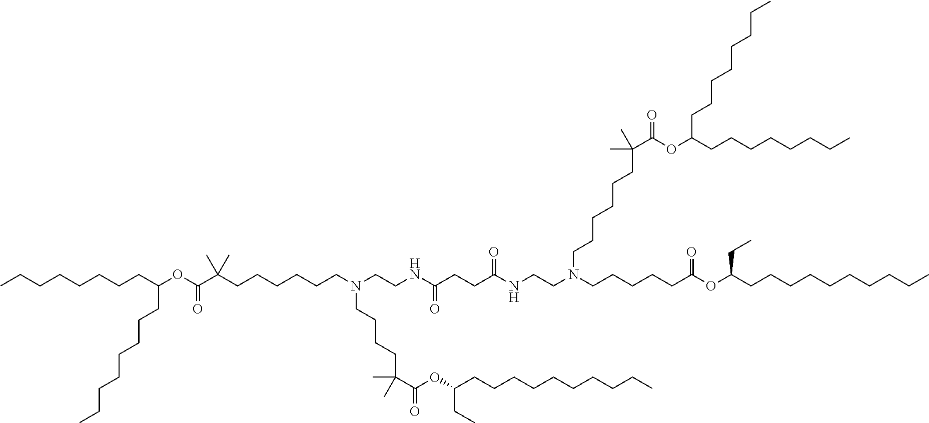 [SMILES: CCCCCCCCCC[C@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@@H](CC)CCCCCCCCCC] | di(heptadecan-9-yl) 9-(5,5-dimethyl-6- oxo-6-(((S)-tridecan- 3-yl)oxy)hexyl)- 2,2,27,27- tetramethyl-13,16- dioxo-20-(6-oxo-6- (((S)-tridecan-3- yl)oxy)hexyl)- 9,12,17,20- tetraaza- octacosanedioate |
| A16 | 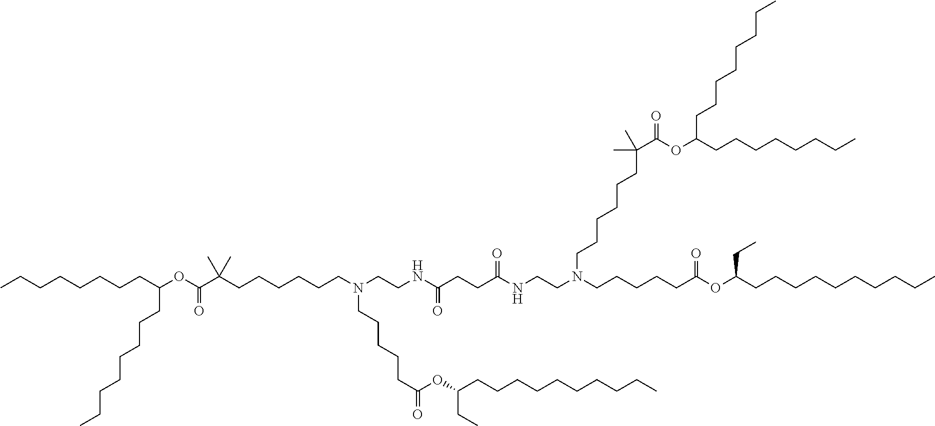 [SMILES: CCCCCCCCCC[C@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@@H](CC)CCCCCCCCCC] | di(heptadecan-9-yl) 2,2,27,27- tetramethyl-13,16- dioxo-9,20-bis(6- oxo-6-(((S)-tridecan- 3-yl)oxy)hexyl)- 9,12,17,20- tetraaza- octacosanedioate |
| A17 | 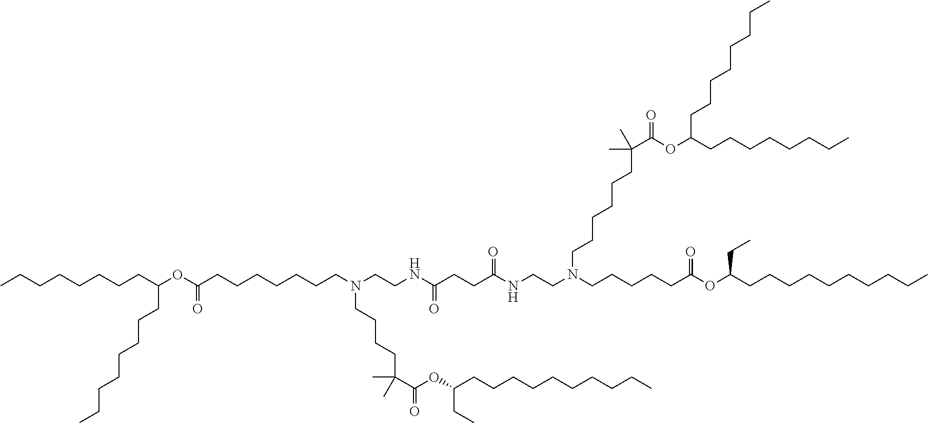 [SMILES: CCCCCCCCCC[C@H](CC)OC(=O)CCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@@H](CC)CCCCCCCCCC] | di(heptadecan-9-yl) 20-(5,5-dimethyl-6- oxo-6-(((S)-tridecan- 3-yl)oxy)hexyl)-2,2- dimethyl-13,16- dioxo-9-(6-oxo-6- (((S)-tridecan-3- yl)oxy)hexyl)- 9,12,17,20- tetraaza- octacosanedioate |
| A18 | 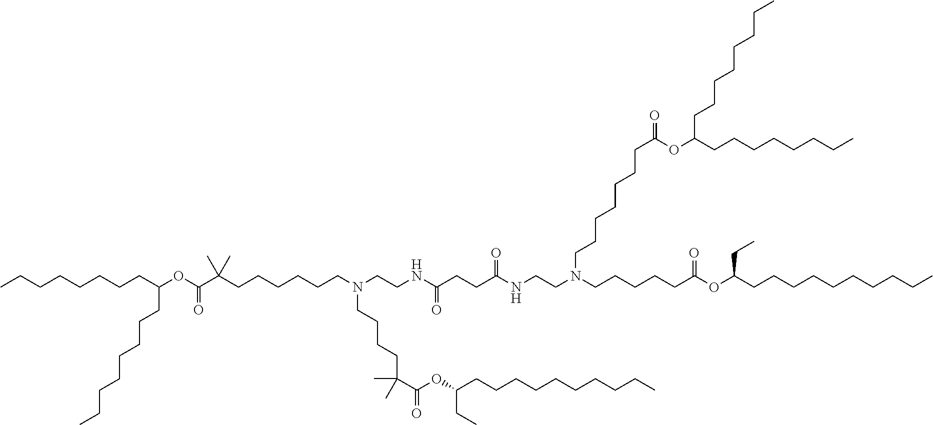 [SMILES: CCCCCCCCCC[C@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@@H](CC)CCCCCCCCCC] | di(heptadecan-9-yl) 9-(5,5-dimethyl-6- oxo-6-(((S)-tridecan- 3-yl)oxy)hexyl)-2,2- dimethyl-13,16- dioxo-20-(6-oxo-6- (((S)-tridecan-3- yl)oxy)hexyl)- 9,12,17,20- tetraaza- octacosanedioate |
| A19 | 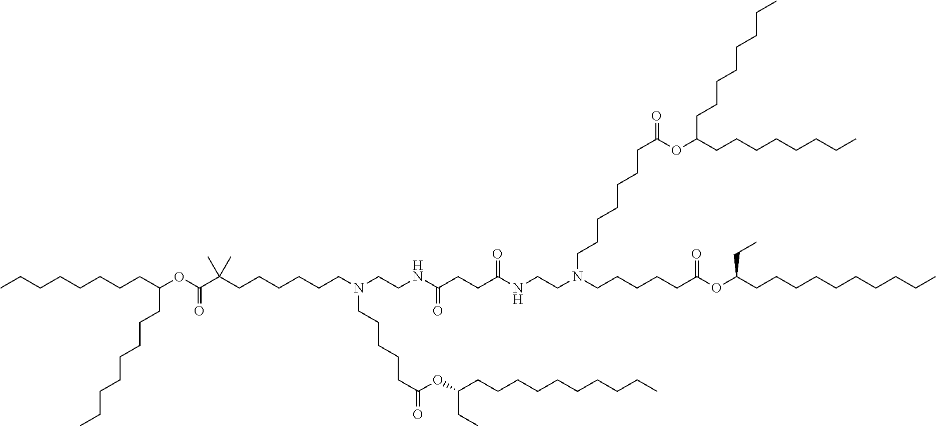 [SMILES: CCCCCCCCCC[C@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@@H](CC)CCCCCCCCCC] | di(heptadecan-9-yl) 2,2-dimethyl-13,16- dioxo-9,20-bis(6- oxo-6-(((S)-tridecan- 3-yl)oxy)hexyl)- 9,12,17,20- tetraaza- octacosanedioate |
| A20 | 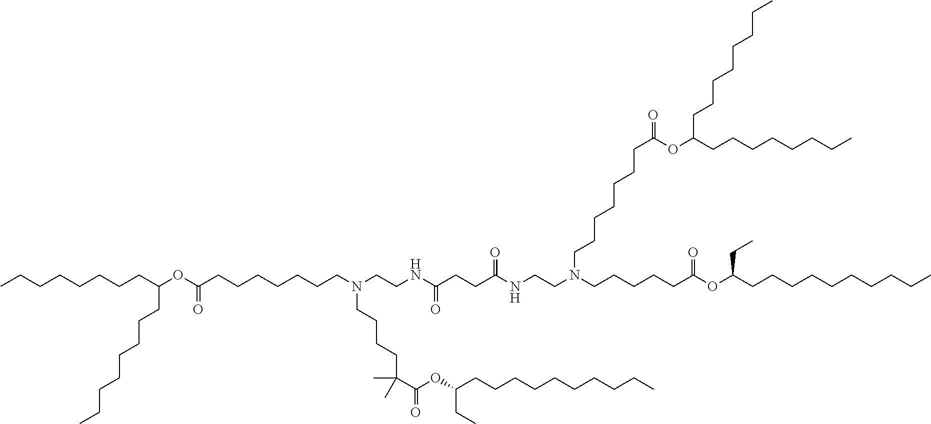 [SMILES: CCCCCCCCCC[C@H](CC)OC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@@H](CC)CCCCCCCCCC] | di(heptadecan-9-yl) 9-(5,5-dimethyl-6- oxo-6-(((S)-tridecan- 3-yl)oxy)hexyl)- 13,16-dioxo-20-(6- oxo-6-(((S)-tridecan- 3-yl)oxy)hexyl)- 9,12,17,20- tetraaza- octacosanedioate |
| A21 | 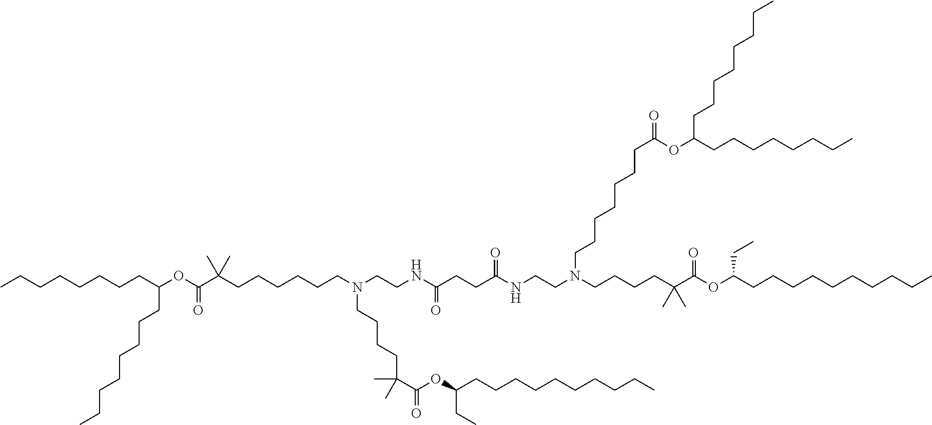 [SMILES: CCCCCCCCCC[C@@H](CC)OC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] | di(heptadecan-9-yl) 9,20-bis(5,5- dimethyl-6-oxo-6- (((R)-tridecan-3- yl)oxy)hexyl)-2,2- dimethyl-13,16- dioxo-9,12,17,20- tetraaza- octacosanedioate |
| A22 | 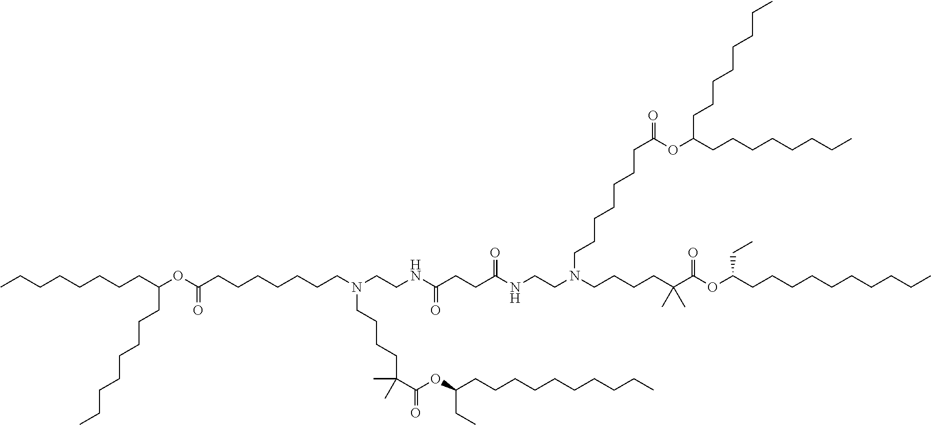 [SMILES: CCCCCCCCCC[C@@H](CC)OC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@H](CC)CCCCCCCCCC] | di(heptadecan-9-yl) 9,20-bis(5,5- dimethyl-6-oxo-6- (((R)-tridecan-3- yl)oxy)hexyl)-13,16- dioxo-9,12,17,20- tetraaza- octacosanedioate |
| A23 | 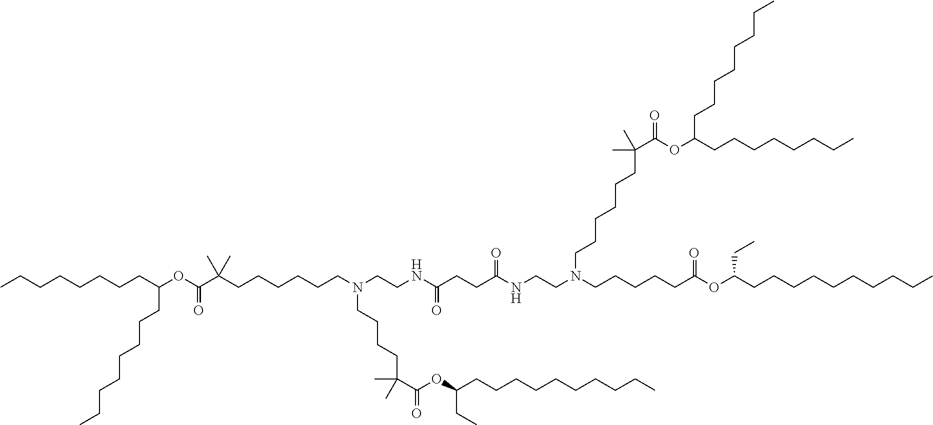 [SMILES: CCCCCCCCCC[C@@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] | di(heptadecan-9-yl) 9-(5,5-dimethyl-6- oxo-6-(((R)-tridecan- 3-yl)oxy)hexyl)- 2,2,27,27- tetramethyl-13,16- dioxo-20-(6-oxo-6- (((R)-tridecan-3- yl)oxy)hexyl)- 9,12,17,20- tetraaza- octacosanedioate |
| A24 | 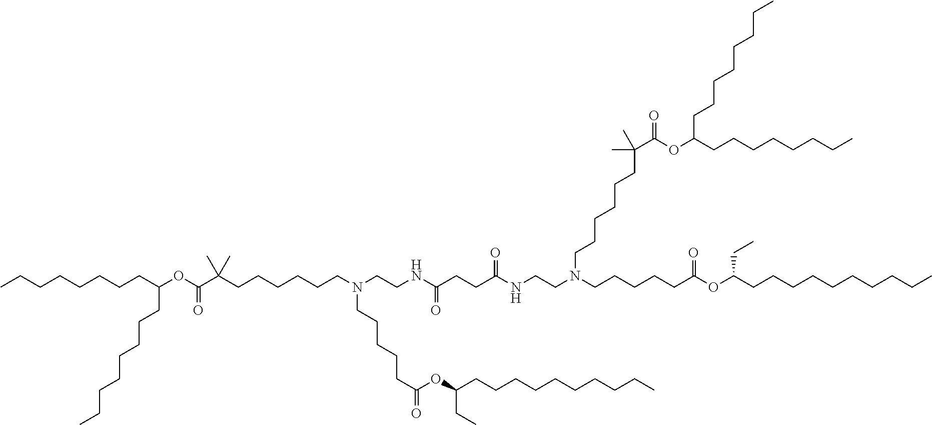 [SMILES: CCCCCCCCCC[C@@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@H](CC)CCCCCCCCCC] | di(heptadecan-9-yl) 2,2,27,27- tetramethyl-13,16- dioxo-9,20-bis(6- oxo-6-(((R)-tridecan- 3-yl)oxy)hexyl)- 9,12,17,20- tetraaza- octacosanedioate |
| A25 | 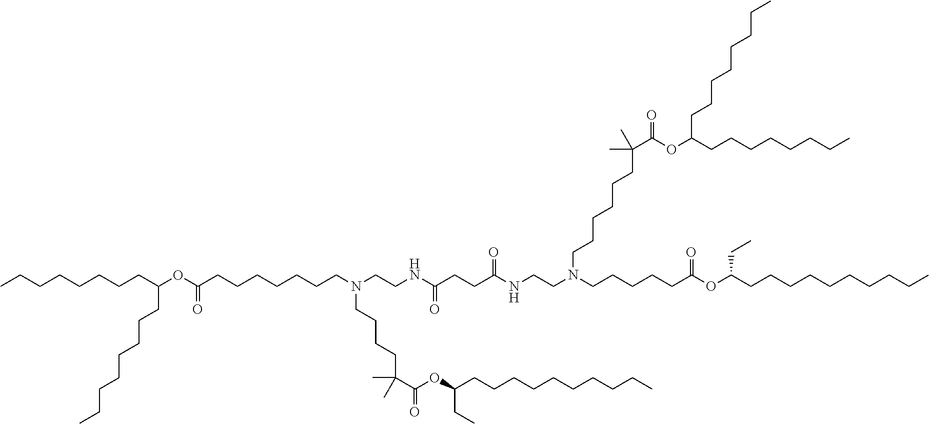 [SMILES: CCCCCCCCCC[C@@H](CC)OC(=O)CCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@H](CC)CCCCCCCCCC] | di(heptadecan-9-yl) 20-(5,5-dimethyl-6- oxo-6-(((R)-tridecan- 3-yl)oxy)hexyl)-2,2- dimethyl-13,16- dioxo-9-(6-oxo-6- (((R)-tridecan-3- yl)oxy)hexyl)- 9,12,17,20- tetraaza- octacosanedioate |
| A26 | 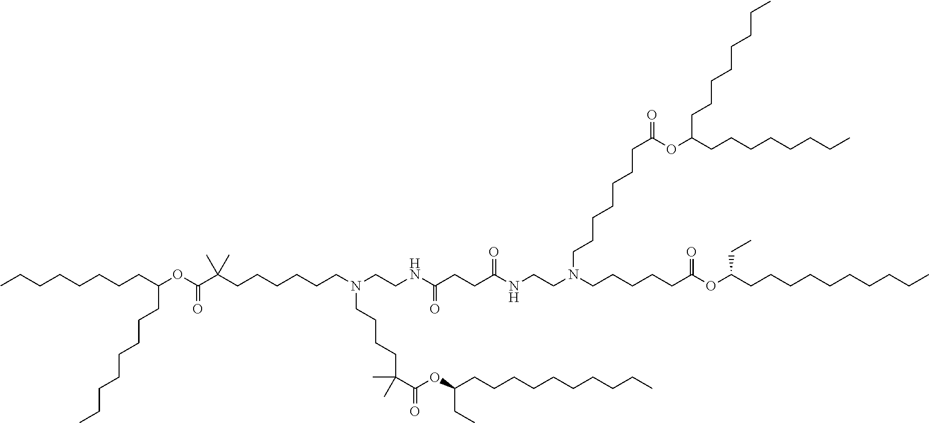 [SMILES: CCCCCCCCCC[C@@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] | di(heptadecan-9-yl) 9-(5,5-dimethyl-6- oxo-6-(((R)-tridecan- 3-yl)oxy)hexyl)-2,2- dimethyl-13,16- dioxo-20-(6-oxo-6- (((R)-tridecan-3- yl)oxy)hexyl)- 9,12,17,20- tetraaza- octacosanedioate |
| A27 | 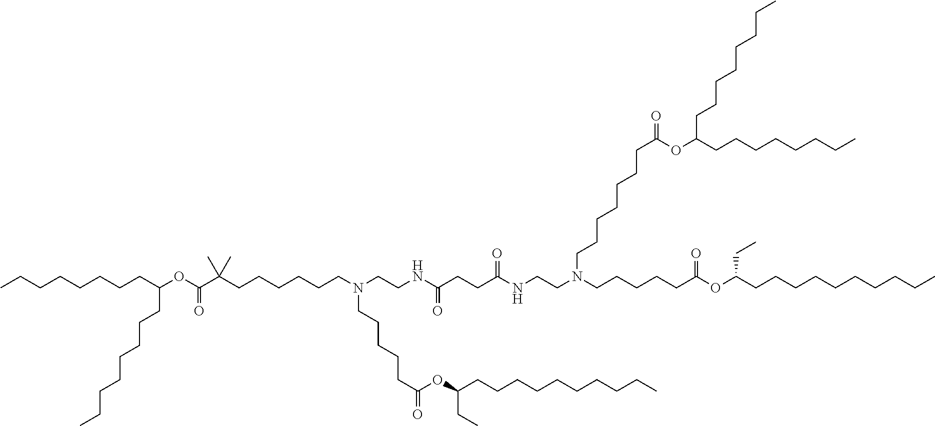 [SMILES: CCCCCCCCCC[C@@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@H](CC)CCCCCCCCCC] | di(heptadecan-9-yl) 2,2-dimethyl-13,16- dioxo-9,20-bis(6- oxo-6-(((R)-tridecan- 3-yl)oxy)hexyl)- 9,12,17,20- tetraaza- octacosanedioate |
| A28 | 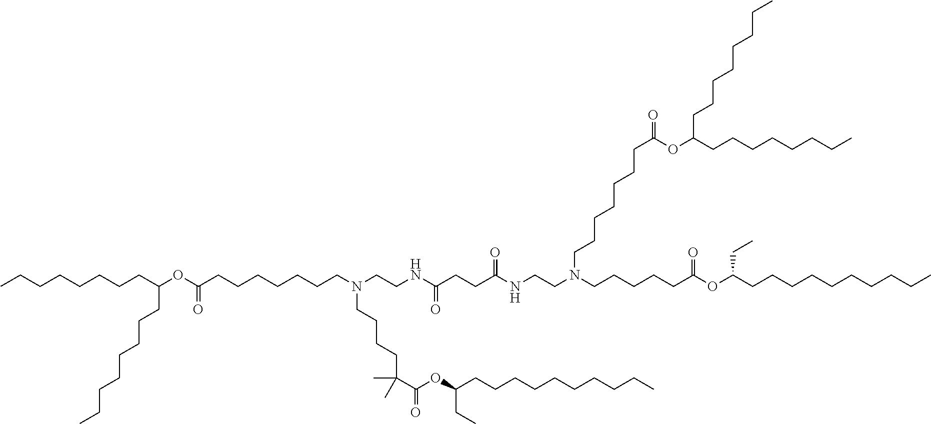 [SMILES: CCCCCCCCCC[C@@H](CC)OC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@H](CC)CCCCCCCCCC] | di(heptadecan-9-yl) 9-(5,5-dimethyl-6- oxo-6-(((R)-tridecan- 3-yl)oxy)hexyl)- 13,16-dioxo-20-(6- oxo-6-(((R)-tridecan- 3-yl)oxy)hexyl)- 9,12,17,20- tetraaza- octacosanedioate |
| A29 | 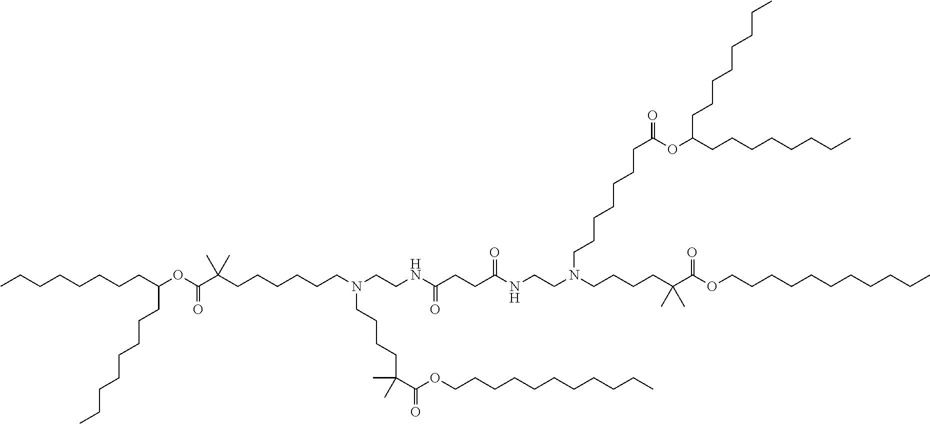 [SMILES: CCCCCCCCCCCOC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9,20-bis(5,5- dimethyl-6-oxo-6- (undecyloxy)hexyl)- 2,2-dimethyl-13,16- dioxo-9,12,17,20- tetraaza- octacosanedioate |
| A30 | 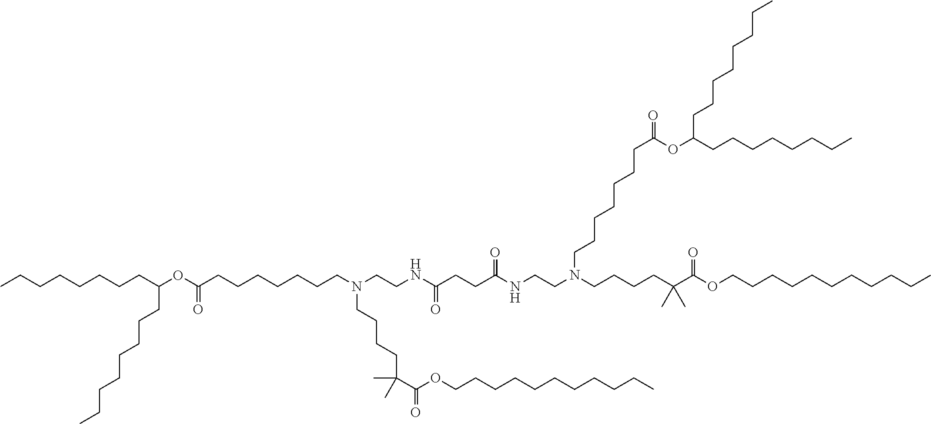 [SMILES: CCCCCCCCCCCOC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OCCCCCCCCCCC] | di(heptadecan-9-yl) 9,20-bis(5,5- dimethyl-6-oxo-6- (undecyloxy)hexyl)- 13,16-dioxo- 9,12,17,20- tetraaza- octacosanedioate |
| A31 | 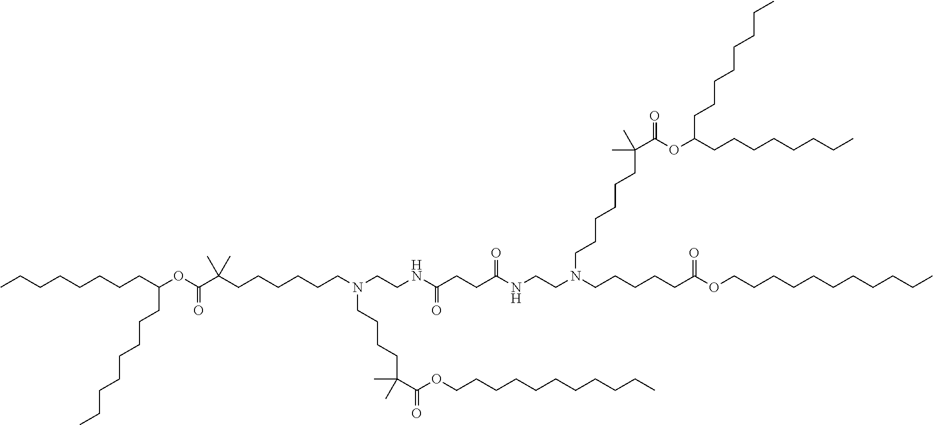 [SMILES: CCCCCCCCCCCOC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9-(5,5-dimethyl-6- oxo-6- (undecyloxy)hexyl)- 2,2,27,27- tetramethyl-13,16- dioxo-20-(6-oxo-6- (undecyloxy)hexyl)- 9,12,17,20- tetraaza- octacosanedioate |
| A32 | 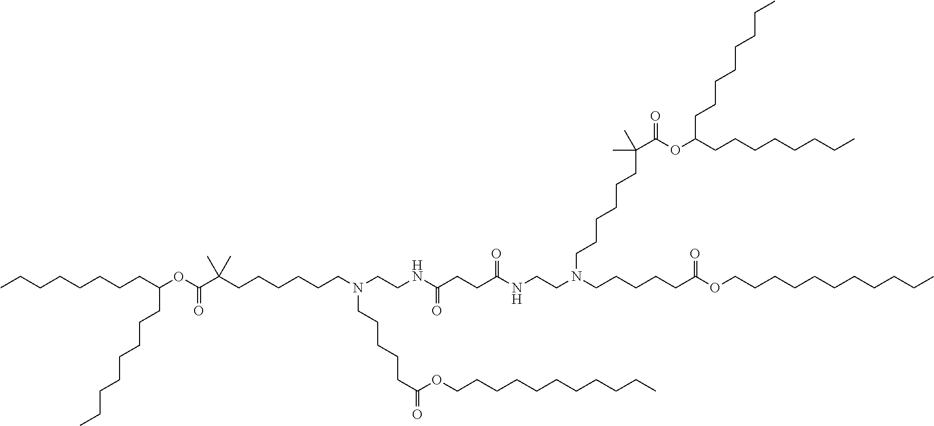 [SMILES: CCCCCCCCCCCOC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)OCCCCCCCCCCC] | di(heptadecan-9-yl) 2,2,27,27- tetramethyl-13,16- dioxo-9,20-bis(6- oxo-6- (undecyloxy)hexyl)- 9,12,17,20- tetraaza- octacosanedioate |
| A33 | 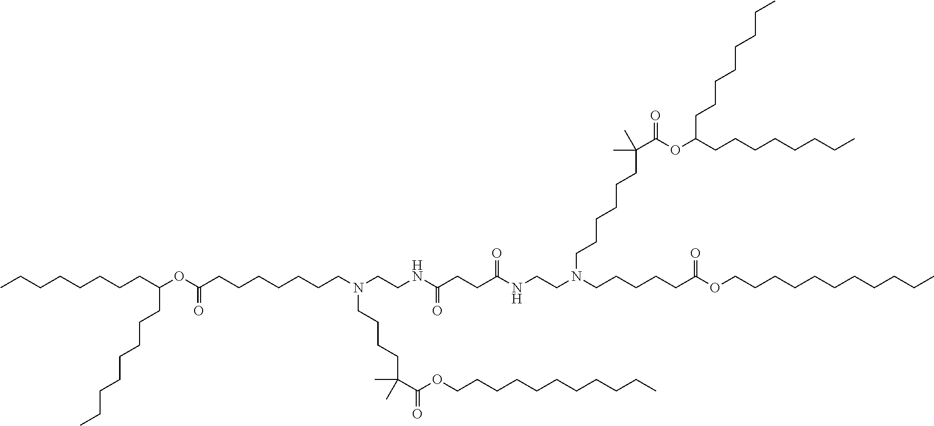 [SMILES: CCCCCCCCCCCOC(=O)CCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OCCCCCCCCCCC] | di(heptadecan-9-yl) 20-(5,5-dimethyl-6- oxo-6- (undecyloxy)hexyl)- 2,2-dimethyl-13,16- dioxo-9-(6-oxo-6- (undecyloxy)hexyl)- 9,12,17,20- tetraaza- octacosanedioate |
| A34 | 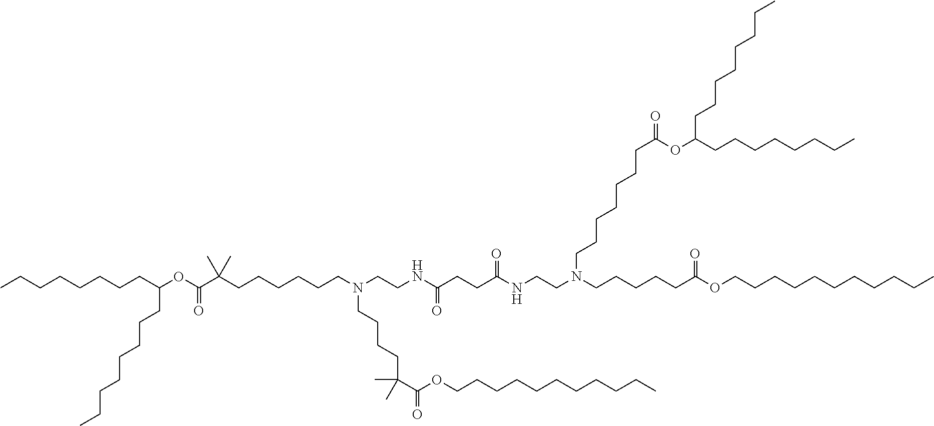 [SMILES: CCCCCCCCCCCOC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9-(5,5-dimethyl-6- oxo-6- (undecyloxy)hexyl)- 2,2-dimethyl-13,16- dioxo-20-(6-oxo-6- (undecyloxy)hexyl)- 9,12,17,20- tetraaza- octacosanedioate |
| A35 | 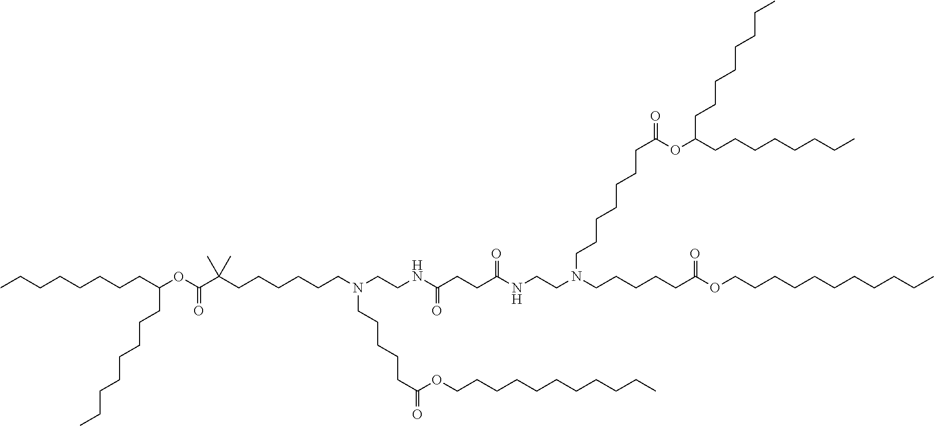 [SMILES: CCCCCCCCCCCOC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)OCCCCCCCCCCC] | di(heptadecan-9-yl) 2,2-dimethyl-13,16- dioxo-9,20-bis(6- oxo-6- (undecyloxy)hexyl)- 9,12,17,20- tetraaza- octacosanedioate |
| A36 | 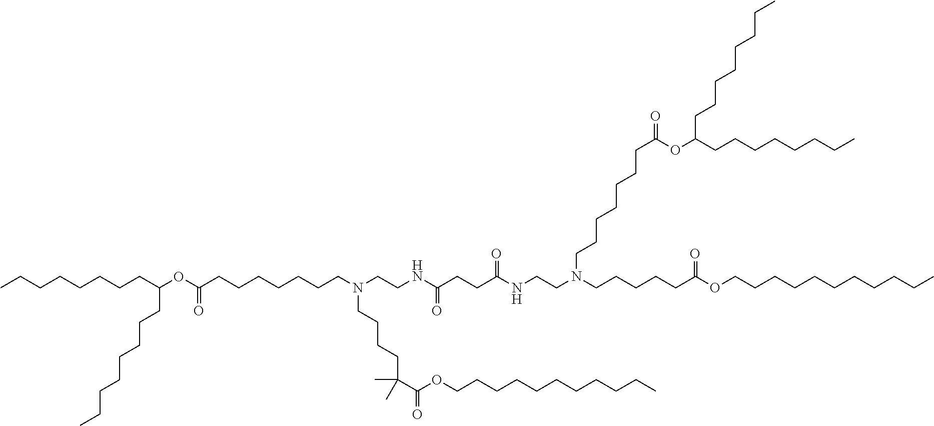 [SMILES: CCCCCCCCCCCOC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OCCCCCCCCCCC] | di(heptadecan-9-yl) 9-(5,5-dimethyl-6- oxo-6- (undecyloxy)hexyl)- 13,16-dioxo-20-(6- oxo-6- (undecyloxy)hexyl)- 9,12,17,20- tetraaza- octacosanedioate |
| A37 | 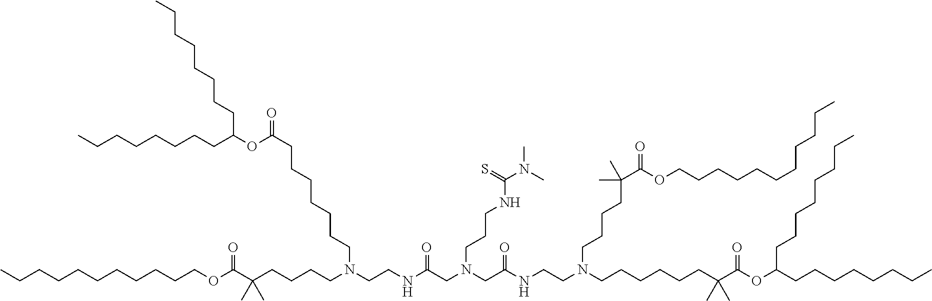 [SMILES: CCCCCCCCCCCOC(=O)C(C)(C)CCCCN(CCCCCCCC(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9,21-bis(5,5- dimethyl-6-oxo-6- (undecyloxy)hexyl)- 15-(3-(3,3- dimethylthioureido) propyl)-2,2-dimethyl- 13,17-dioxo- 9,12,15,18,21- pentaaza- nonacosanedioate |
| A38 | 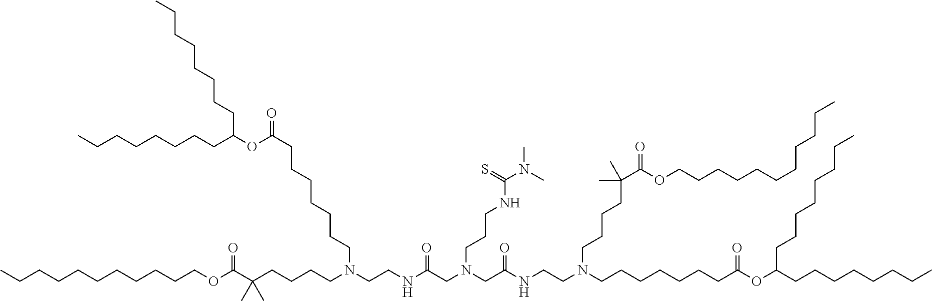 [SMILES: CCCCCCCCCCCOC(=O)C(C)(C)CCCCN(CCCCCCCC(=O)OC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCC(C)(C)C(=O)OCCCCCCCCCCC] | di(heptadecan-9-yl) 9,21-bis(5,5- dimethyl-6-oxo-6- (undecyloxy)hexyl)- 15-(3-(3,3- dimethylthioureido) propyl)-13,17-dioxo- 9,12,15,18,21- pentaaza- nonacosanedioate |
| A39 | 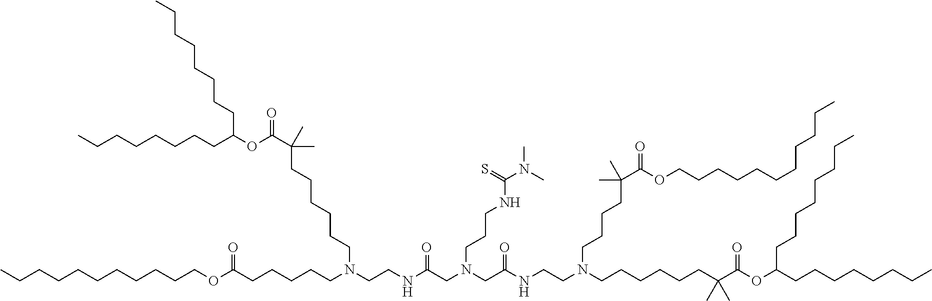 [SMILES: CCCCCCCCCCCOC(=O)CCCCCN(CCCCCCC(C)(C)C(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9-(5,5-dimethyl-6- oxo-6- (undecyloxy)hexyl)- 15-(3-(3,3- dimethylthioureido) propyl)-2,2,28,28- tetramethyl-13,17- dioxo-21-(6-oxo-6- (undecyloxy)hexyl)- 9,12,15,18,21- pentaaza- nonacosanedioate |
| A40 | 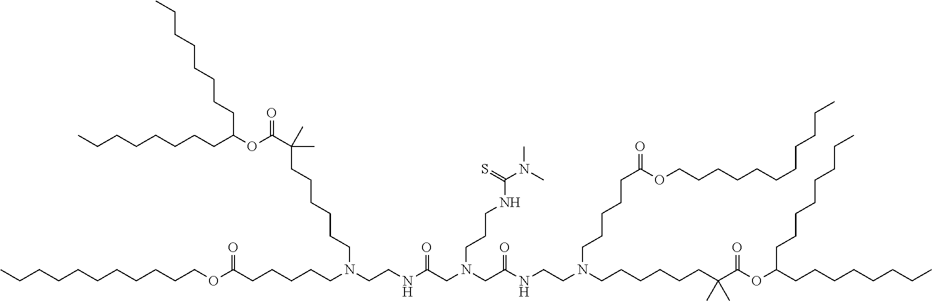 [SMILES: CCCCCCCCCCCOC(=O)CCCCCN(CCCCCCC(C)(C)C(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCCC(=O)OCCCCCCCCCCC] | di(heptadecan-9-yl) 15-(3-(3,3- dimethylthioureido) propyl)-2,2,28,28- tetramethyl-13,17- dioxo-9,21-bis(6- oxo-6- (undecyloxy)hexyl)- 9,12,15,18,21- pentaaza- nonacosanedioate |
| A41 | 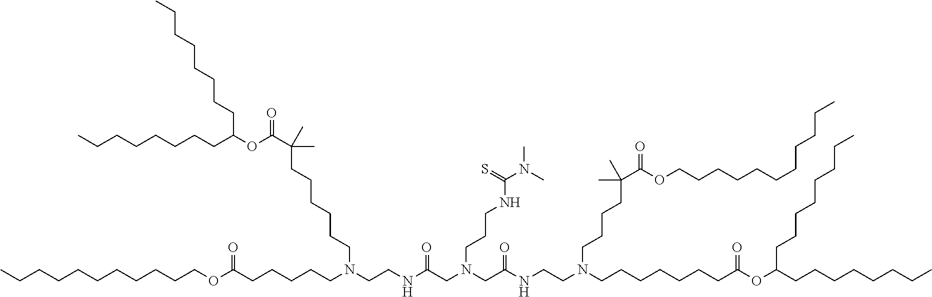 [SMILES: CCCCCCCCCCCOC(=O)CCCCCN(CCCCCCC(C)(C)C(=O)OC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCC(C)(C)C(=O)OCCCCCCCCCCC] | di(heptadecan-9-yl) 21-(5,5-dimethyl-6- oxo-6- (undecyloxy)hexyl)- 15-(3-(3,3- dimethylthioureido) propyl)-2,2-dimethyl- 13,17-dioxo-9-(6- oxo-6- (undecyloxy)hexyl)- 9,12,15,18,21- pentaaza- nonacosanedioate |
| A42 | 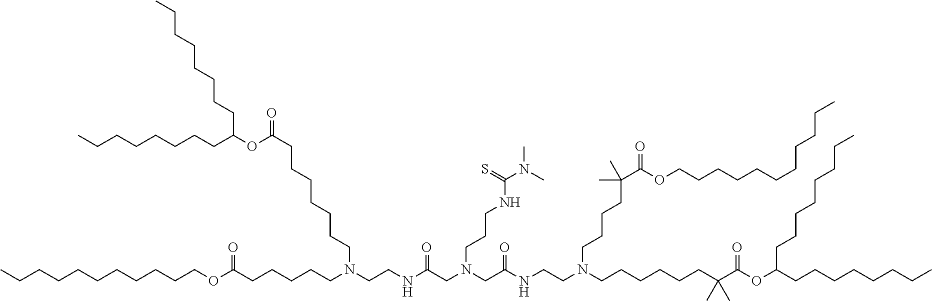 [SMILES: CCCCCCCCCCCOC(=O)CCCCCN(CCCCCCCC(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9-(5,5-dimethyl-6- oxo-6- (undecyloxy)hexyl)- 15-(3-(3,3- dimethylthioureido) propyl)-2,2-dimethyl- 13,17-dioxo-21-(6- oxo-6- (undecyloxy)hexyl)- 9,12,15,18,21- pentaaza- nonacosanedioate |
| A43 | 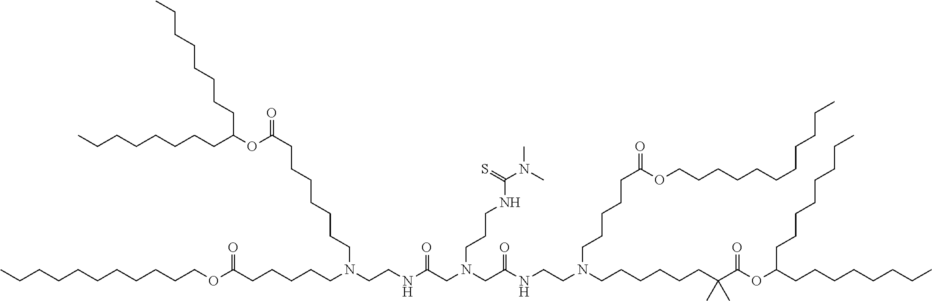 [SMILES: CCCCCCCCCCCOC(=O)CCCCCN(CCCCCCCC(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCCC(=O)OCCCCCCCCCCC] | di(heptadecan-9-yl) 15-(3-(3,3- dimethylthioureido) propyl)-2,2-dimethyl- 13,17-dioxo-9,21- bis(6-oxo-6- (undecyloxy)hexyl)- 9,12,15,18,21- pentaaza- nonacosanedioate |
| A44 | 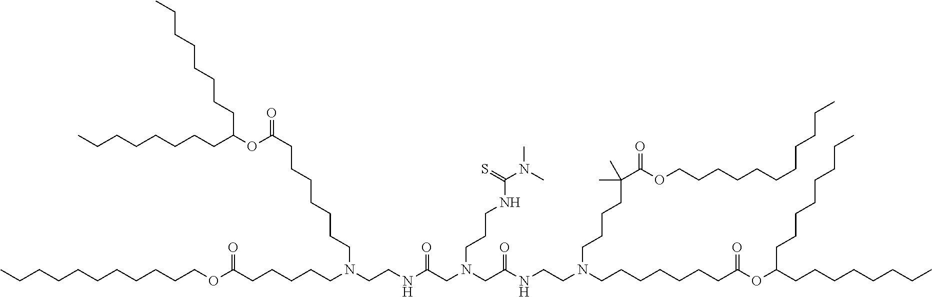 [SMILES: CCCCCCCCCCCOC(=O)CCCCCN(CCCCCCCC(=O)OC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCC(C)(C)C(=O)OCCCCCCCCCCC] | di(heptadecan-9-yl) 9-(5,5-dimethyl-6- oxo-6- (undecyloxy)hexyl)- 15-(3-(3,3- dimethylthioureido) propyl)-13,17-dioxo- 21-(6-oxo-6- (undecyloxy)hexyl)- 9,12,15,18,21- pentaaza- nonacosanedioate |
| A45 | 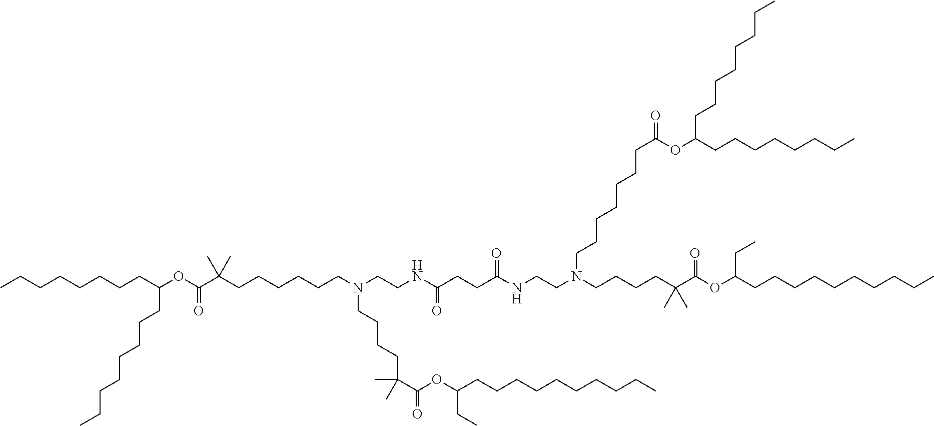 [SMILES: CCCCCCCCCCC(CC)OC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OC(CC)CCCCCCCCCC] | di(heptadecan-9-yl) 9,20-bis(5,5- dimethyl-6-oxo-6- (tridecan-3- yloxy)hexyl)-2,2- dimethyl-13,16- dioxo-9,12,17,20- tetraaza- octacosanedioate |
| A46 | 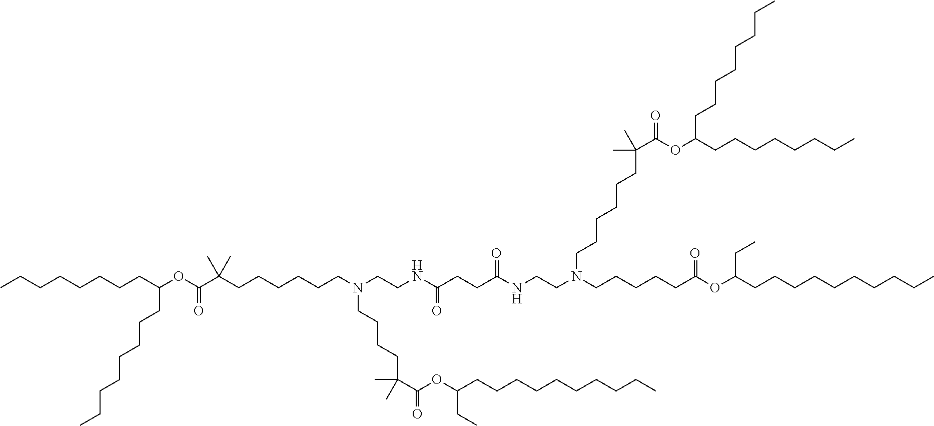 [SMILES: CCCCCCCCCCC(CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OC(CC)CCCCCCCCCC] | di(heptadecan-9-yl) 9-(5,5-dimethyl-6- oxo-6-(tridecan-3- yloxy)hexyl)- 2,2,27,27- tetramethyl-13,16- dioxo-20-(6-oxo-6- (tridecan-3- yloxy)hexyl)- 9,12,17,20- tetraaza- octacosanedioate |
| A47 | 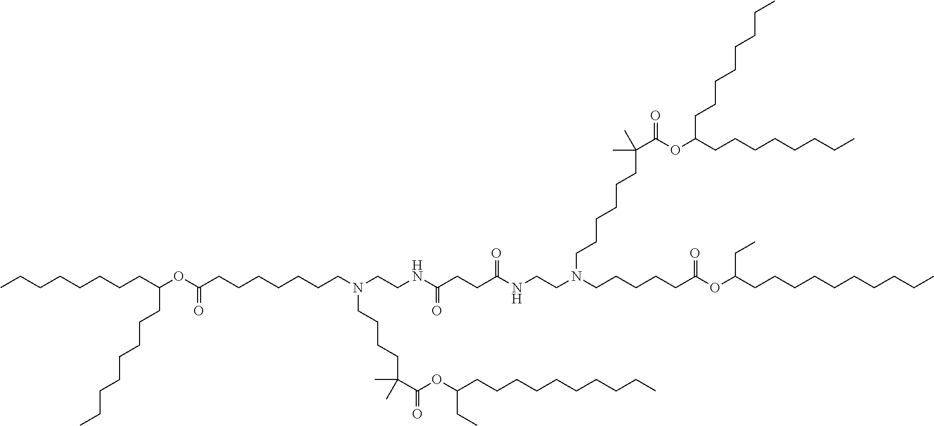 [SMILES: CCCCCCCCCCC(CC)OC(=O)CCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OC(CC)CCCCCCCCCC] | di(heptadecan-9-yl) 20-(5,5-dimethyl-6- oxo-6-(tridecan-3- yloxy)hexyl)-2,2- dimethyl-13,16- dioxo-9-(6-oxo-6- (tridecan-3- yloxy)hexyl)- 9,12,17,20- tetraaza- octacosanedioate |
| A48 | 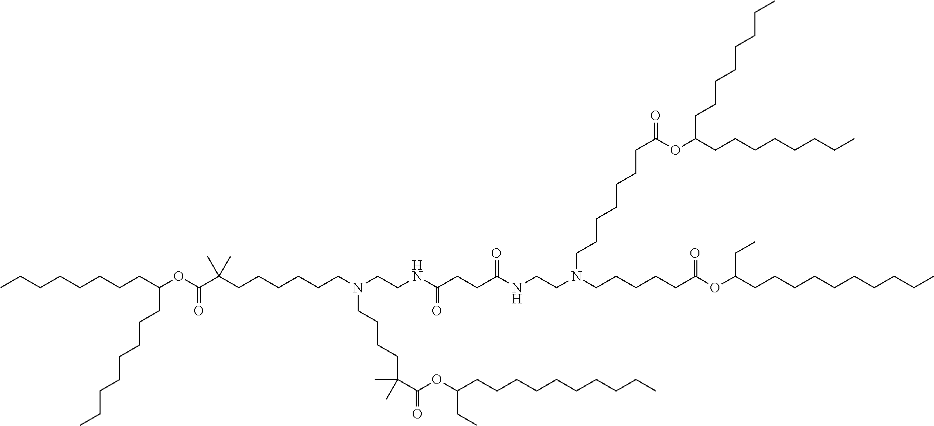 [SMILES: CCCCCCCCCCC(CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OC(CC)CCCCCCCCCC] | di(heptadecan-9-yl) 9-(5,5-dimethyl-6- oxo-6-(tridecan-3- yloxy)hexyl)-2,2- dimethyl-13,16- dioxo-20-(6-oxo-6- (tridecan-3- yloxy)hexyl)- 9,12,17,20- tetraaza- octacosanedioate |
| A49 | 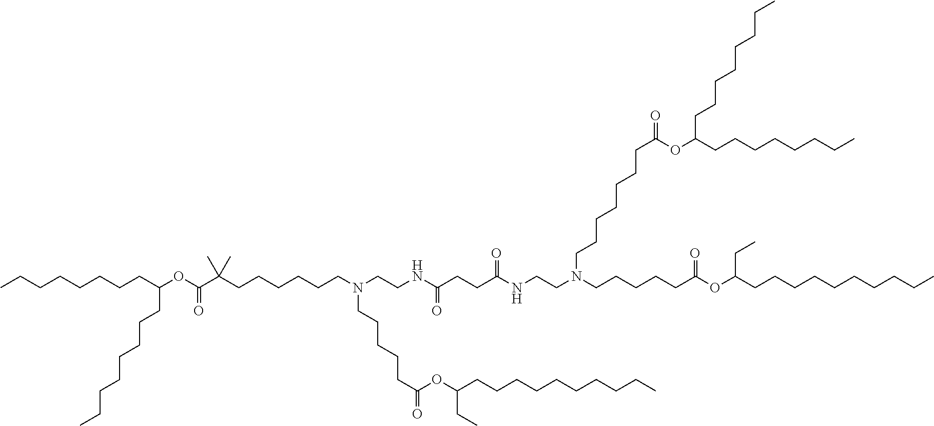 [SMILES: CCCCCCCCCCC(CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)OC(CC)CCCCCCCCCC] | di(heptadecan-9-yl) 2,2-dimethyl-13,16- dioxo-9,20-bis(6- oxo-6-(tridecan-3- yloxy)hexyl)- 9,12,17,20- tetraaza- octacosanedioate |
| A50 | 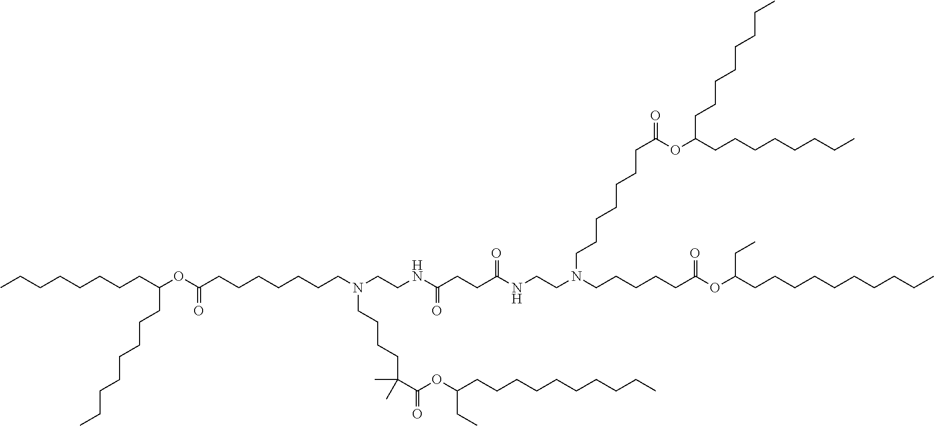 [SMILES: CCCCCCCCCCC(CC)OC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OC(CC)CCCCCCCCCC] | di(heptadecan-9-yl) 9-(5,5-dimethyl-6- oxo-6-(tridecan-3- yloxy)hexyl)-13,16- dioxo-20-(6-oxo-6- (tridecan-3- yloxy)hexyl)- 9,12,17,20- tetraaza- octacosanedioate |
| A51 | 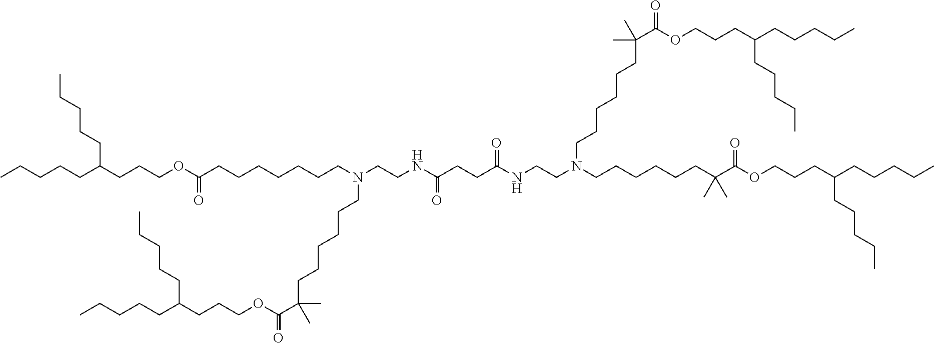 [SMILES: CCCCCC(CCCCC)CCCOC(=O)CCCCCCCN(CCCCCCC(C)(C)C(=O)OCCCC(CCCCC)CCCCC)CCNC(=O)CCC(=O)NCCN(CCCCCCC(C)(C)C(=O)OCCCC(CCCCC)CCCCC)CCCCCCC(C)(C)C(=O)OCCCC(CCCCC)CCCCC] | bis(4-pentylnonyl) 9- (7,7-dimethyl-8-oxo- 8-((4- pentylnonyl)oxy) octyl)-2,2,27,27- tetramethyl-13,16- dioxo-20-(8-oxo-8- ((4- pentylnonyl)oxy) octyl)-9,12,17,20- tetraaza- octacosanedioate |
| A52 | 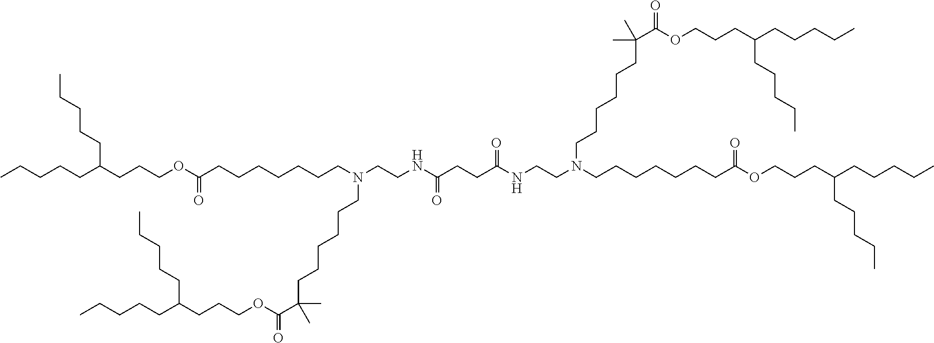 [SMILES: CCCCCC(CCCCC)CCCOC(=O)CCCCCCCN(CCCCCCC(C)(C)C(=O)OCCCC(CCCCC)CCCCC)CCNC(=O)CCC(=O)NCCN(CCCCCCCC(=O)OCCCC(CCCCC)CCCCC)CCCCCCC(C)(C)C(=O)OCCCC(CCCCC)CCCCC] | bis(4-pentylnonyl) 2,2,27,27- tetramethyl-13,16- dioxo-9,20-bis(8- oxo-8-((4- pentylnonyl)oxy) octyl)-9,12,17,20- tetraaza- octacosanedioate |
| A53 | 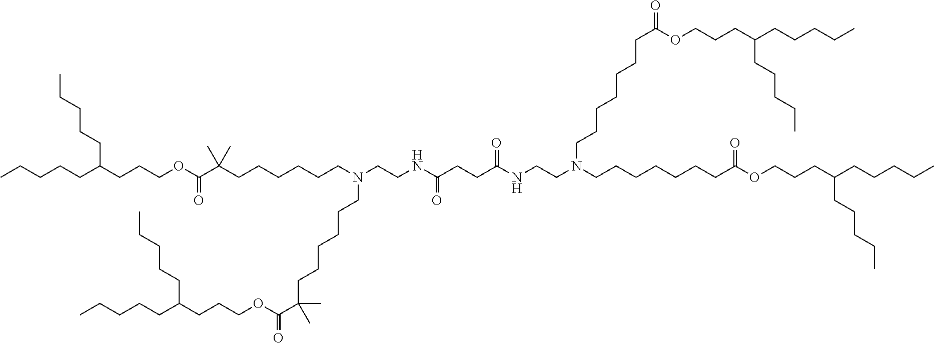 [SMILES: CCCCCC(CCCCC)CCCOC(=O)CCCCCCCN(CCCCCCCC(=O)OCCCC(CCCCC)CCCCC)CCNC(=O)CCC(=O)NCCN(CCCCCCC(C)(C)C(=O)OCCCC(CCCCC)CCCCC)CCCCCCC(C)(C)C(=O)OCCCC(CCCCC)CCCCC] | bis(4-pentylnonyl) 9- (7,7-dimethyl-8-oxo- 8-((4- pentylnonyl)oxy) octyl)-2,2-dimethyl- 13,16-dioxo-20-(8- oxo-8-((4- pentylnonyl)oxy) octyl)-9,12,17,20- tetraaza- octacosanedioate |
| A54 | 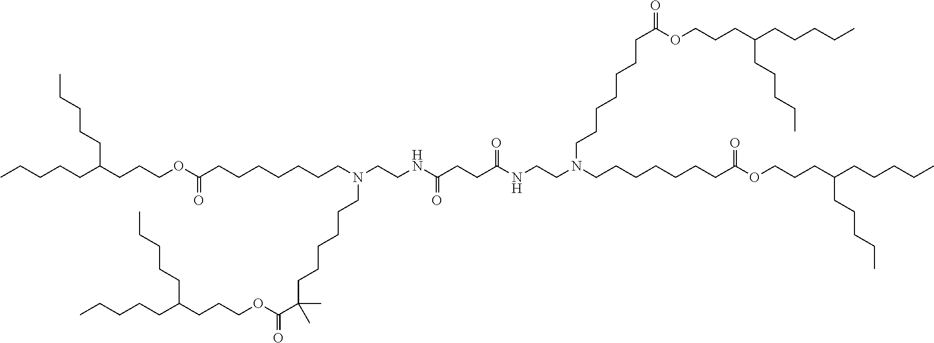 [SMILES: CCCCCC(CCCCC)CCCOC(=O)CCCCCCCN(CCCCCCCC(=O)OCCCC(CCCCC)CCCCC)CCNC(=O)CCC(=O)NCCN(CCCCCCCC(=O)OCCCC(CCCCC)CCCCC)CCCCCCC(C)(C)C(=O)OCCCC(CCCCC)CCCCC] | bis(4-pentylnonyl) 2,2-dimethyl-13,16- dioxo-9,20-bis(8- oxo-8-((4- pentylnonyl)oxy) octyl)-9,12,17,20- tetraaza- octacosanedioate |
| A55 | 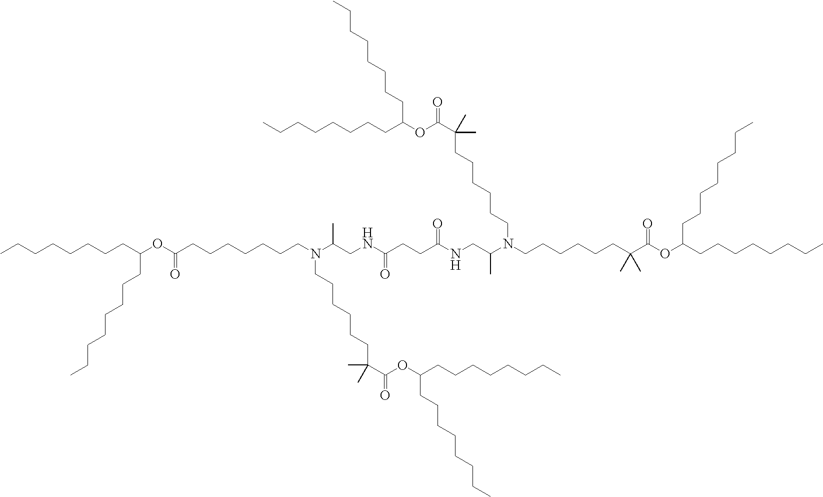 [SMILES: CCCCCCCCC(CCCCCCCC)OC(=O)CCCCCCCN(CCCCCCC(C)(C)C(=O)OC(CCCCCCCC)CCCCCCCC)C(C)CNC(=O)CCC(=O)NCC(C)N(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9-(8-(heptadecan-9- yloxy)-7,7-dimethyl- 8-oxooctyl)-20-(8- (heptadecan-9- yloxy)-8-oxooctyl)- 2,2,10,19,27,27- hexamethyl-13,16- dioxo-9,12,17,20- tetraaza- octacosanedioate |
| A56 | 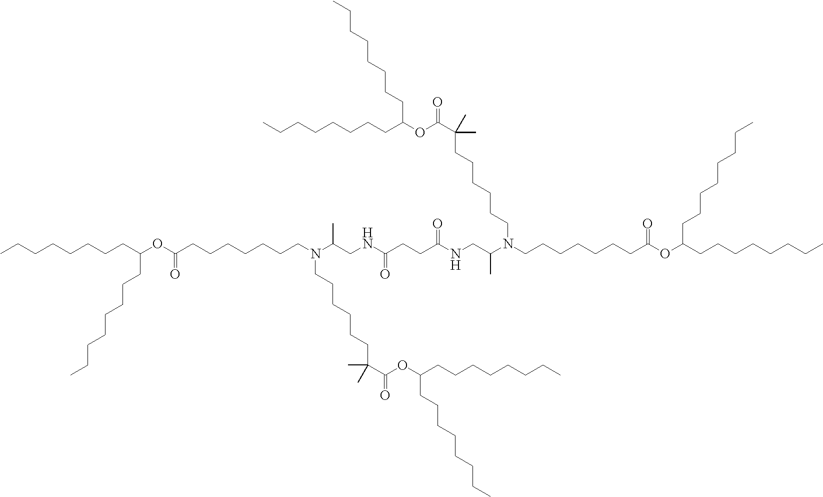 [SMILES: CCCCCCCCC(CCCCCCCC)OC(=O)CCCCCCCN(CCCCCCC(C)(C)C(=O)OC(CCCCCCCC)CCCCCCCC)C(C)CNC(=O)CCC(=O)NCC(C)N(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,20-bis(8- (heptadecan-9- yloxy)-8-oxooctyl)- 2,2,10,19,27,27- hexamethyl-13,16- dioxo-9,12,17,20- tetraaza- octacosanedioate |
| A57 | 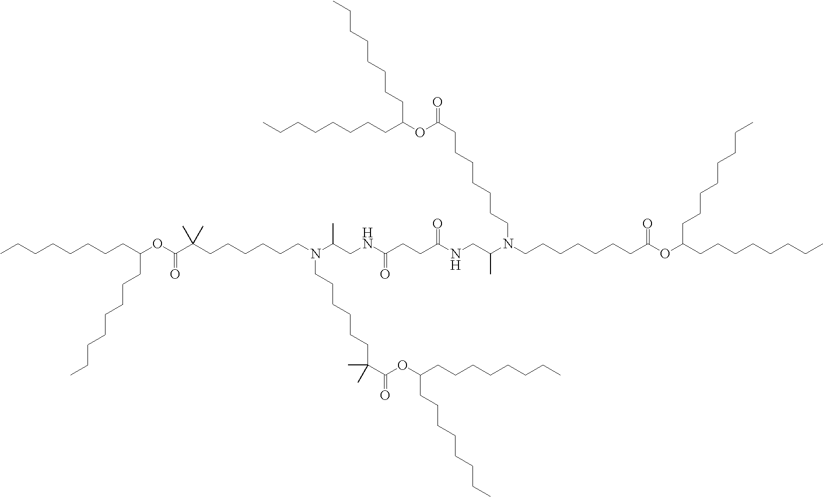 [SMILES: CCCCCCCCC(CCCCCCCC)OC(=O)CCCCCCCN(CCCCCCCC(=O)OC(CCCCCCCC)CCCCCCCC)C(C)CNC(=O)CCC(=O)NCC(C)N(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9-(8-(heptadecan-9- yloxy)-7,7-dimethyl- 8-oxooctyl)-20-(8- (heptadecan-9- yloxy)-8-oxooctyl)- 2,2,10,19- tetramethyl-13,16- dioxo-9,12,17,20- tetraaza- octacosanedioate |
| A58 | 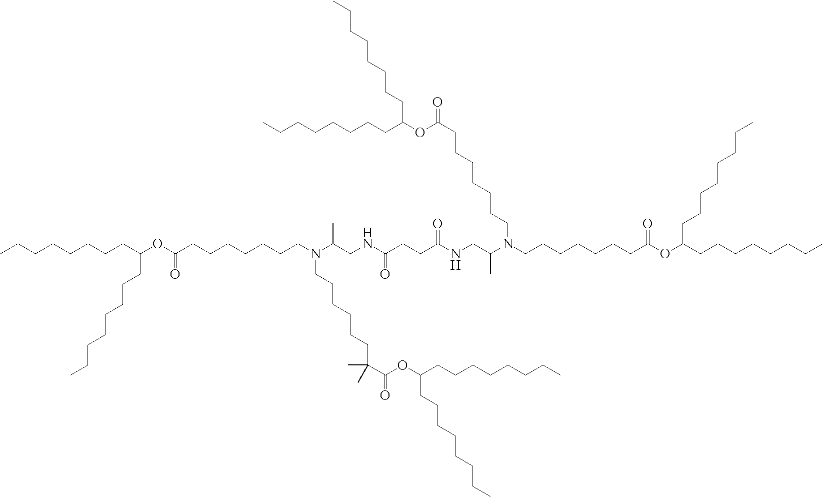 [SMILES: CCCCCCCCC(CCCCCCCC)OC(=O)CCCCCCCN(CCCCCCCC(=O)OC(CCCCCCCC)CCCCCCCC)C(C)CNC(=O)CCC(=O)NCC(C)N(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,20-bis(8- (heptadecan-9- yloxy)-8-oxooctyl)- 2,2,10,19- tetramethyl-13,16- dioxo-9,12,17,20- tetraaza- octacosanedioate |
| A59 | 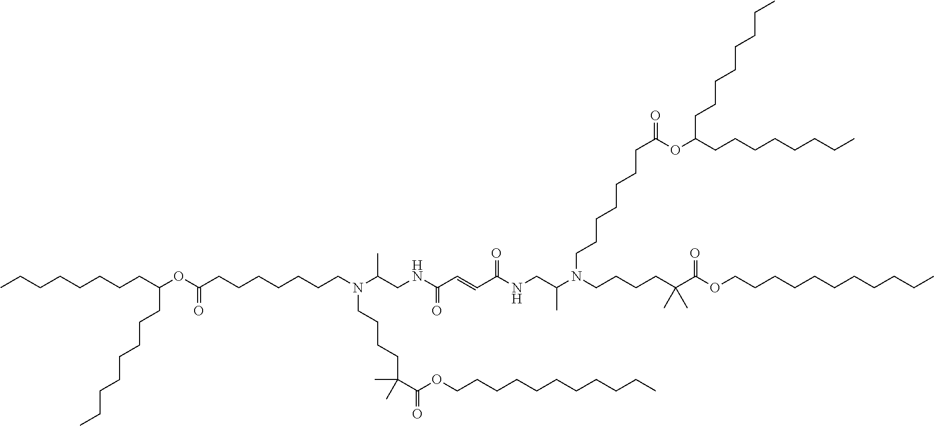 [SMILES: CCCCCCCCCCCOC(=O)C(C)(C)CCCCN(CCCCCCCC(=O)OC(CCCCCCCC)CCCCCCCC)C(C)CNC(=O)/C=C/C(=O)NCC(C)N(CCCCCCCC(=O)OC(CCCCCCCC)CCCCCCCC)CCCCC(C)(C)C(=O)OCCCCCCCCCCC] | di(heptadecan-9-yl) (E)-9,20-bis(5,5- dimethyl-6-oxo-6- (undecyloxy)hexyl)- 10,19-dimethyl- 13,16-dioxo- 9,12,17,20- tetraazaoctacos-14- enedioate |
| A60 | 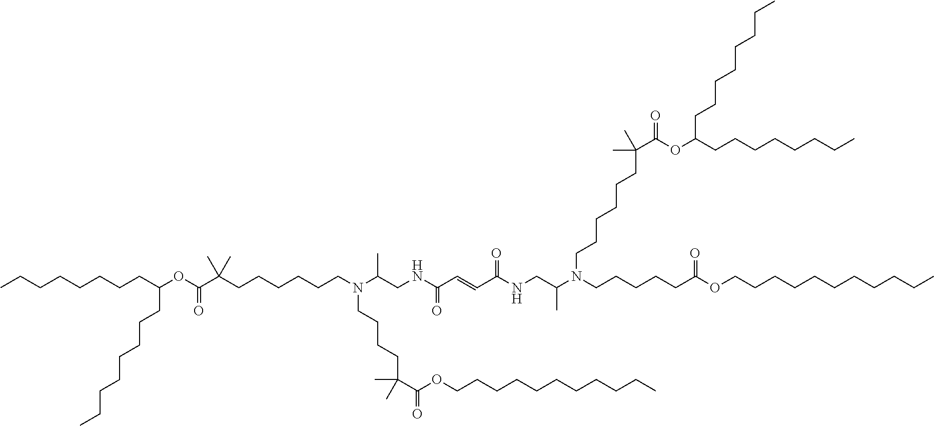 [SMILES: CCCCCCCCCCCOC(=O)CCCCCN(CCCCCCC(C)(C)C(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCC(C)(C)C(=O)OCCCCCCCCCCC] | di(heptadecan-9-yl) (E)-9-(5,5-dimethyl- 6-oxo-6- (undecyloxy)hexyl)- 2,2,10,19,27,27- hexamethyl-13,16- dioxo-20-(6-oxo-6- (undecyloxy)hexyl)- 9,12,17,20- tetraazaoctacos-14- enedioate |
| A61 | 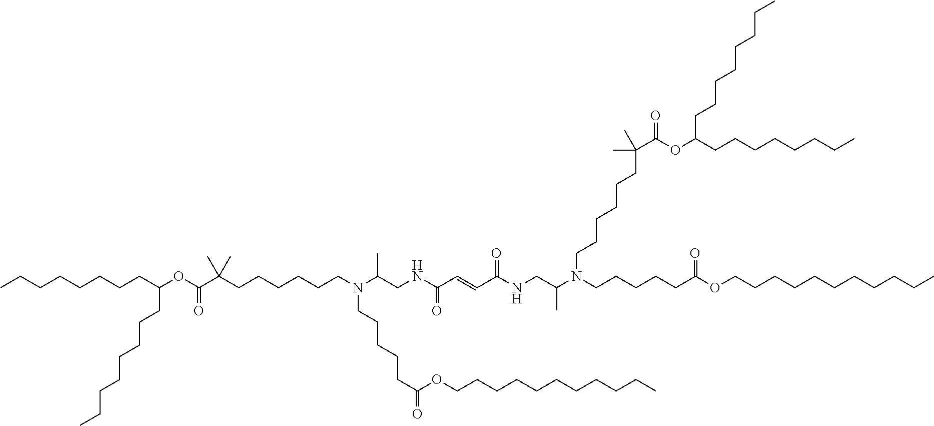 [SMILES: CCCCCCCCCCCOC(=O)CCCCCN(CCCCCCC(C)(C)C(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCCC(=O)OCCCCCCCCCCC] | di(heptadecan-9-yl) (E)-2,2,10,19,27,27- hexamethyl-13,16- dioxo-9,20-bis(6- oxo-6- (undecyloxy)hexyl)- 9,12,17,20- tetraazaoctacos-14- enedioate |
| A62 | 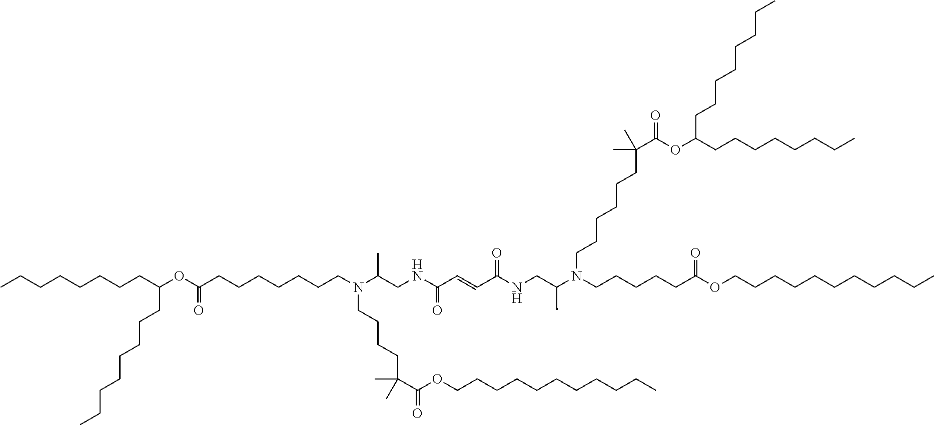 [SMILES: CCCCCCCCCCCOC(=O)CCCCCN(CCCCCCC(C)(C)C(=O)OC(CCCCCCCC)CCCCCCCC)C(C)CNC(=O)/C=C/C(=O)NCC(C)N(CCCCCCCC(=O)OC(CCCCCCCC)CCCCCCCC)CCCCC(C)(C)C(=O)OCCCCCCCCCCC] | di(heptadecan-9-yl) (E)-20-(5,5-dimethyl- 6-oxo-6- (undecyloxy)hexyl)- 2,2,10,19- tetramethyl-13,16- dioxo-9-(6-oxo-6- (undecyloxy)hexyl)- 9,12,17,20- tetraazaoctacos-14- enedioate |
| A63 | 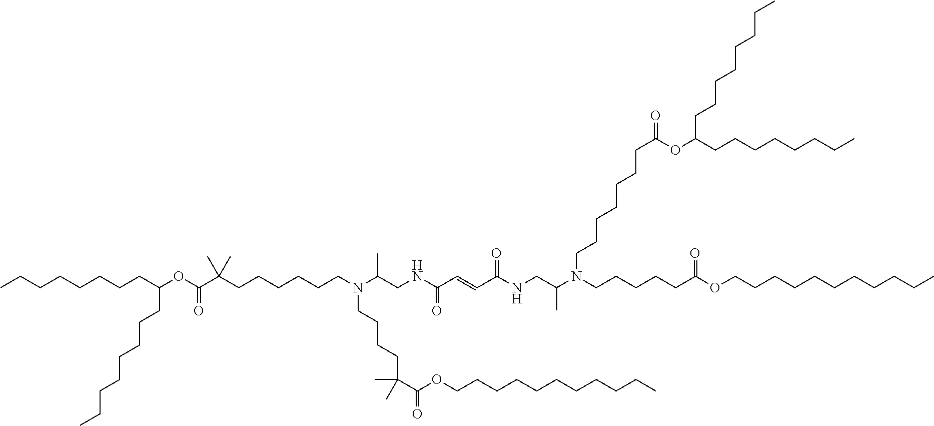 [SMILES: CCCCCCCCCCCOC(=O)CCCCCN(CCCCCCCC(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCC(C)(C)C(=O)OCCCCCCCCCCC] | di(heptadecan-9-yl) (E)-9-(5,5-dimethyl- 6-oxo-6- (undecyloxy)hexyl)- 2,2,10,19- tetramethyl-13,16- dioxo-20-(6-oxo-6- (undecyloxy)hexyl)- 9,12,17,20- tetraazaoctacos-14- enedioate |
| A64 | 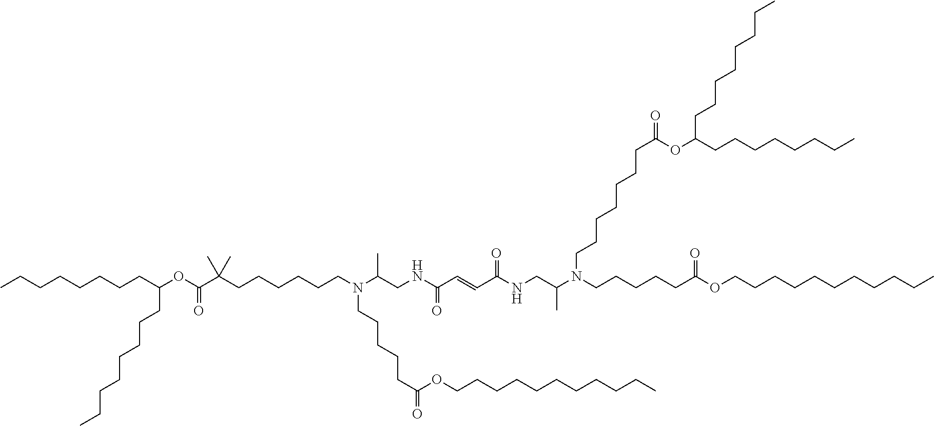 [SMILES: CCCCCCCCCCCOC(=O)CCCCCN(CCCCCCCC(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCCC(=O)OCCCCCCCCCCC] | di(heptadecan-9-yl) (E)-2,2,10,19- tetramethyl-13,16- dioxo-9,20-bis(6- oxo-6- (undecyloxy)hexyl)- 9,12,17,20- tetraazaoctacos-14- enedioate |
| A65 | 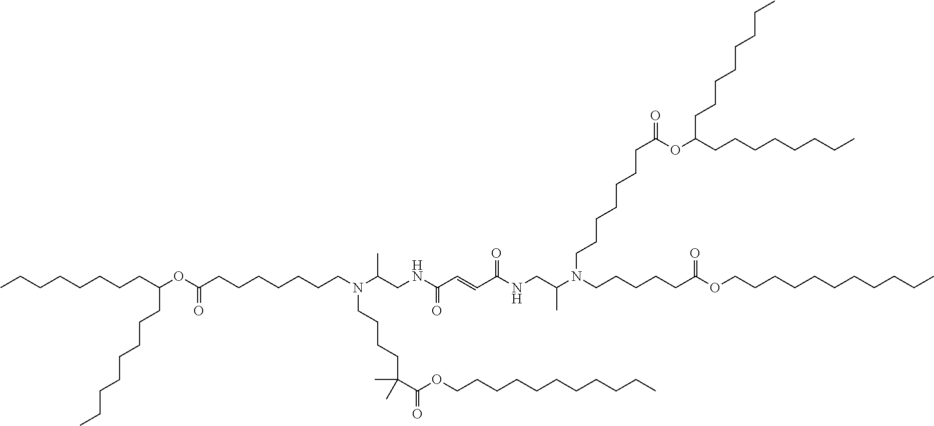 [SMILES: CCCCCCCCCCCOC(=O)CCCCCN(CCCCCCCC(=O)OC(CCCCCCCC)CCCCCCCC)C(C)CNC(=O)/C=C/C(=O)NCC(C)N(CCCCCCCC(=O)OC(CCCCCCCC)CCCCCCCC)CCCCC(C)(C)C(=O)OCCCCCCCCCCC] | di(heptadecan-9-yl) (E)-9-(5,5-dimethyl- 6-oxo-6- (undecyloxy)hexyl)- 10,19-dimethyl- 13,16-dioxo-20-(6- oxo-6- (undecyloxy)hexyl)- 9,12,17,20- tetraazaoctacos-14- enedioate |
| A66 | 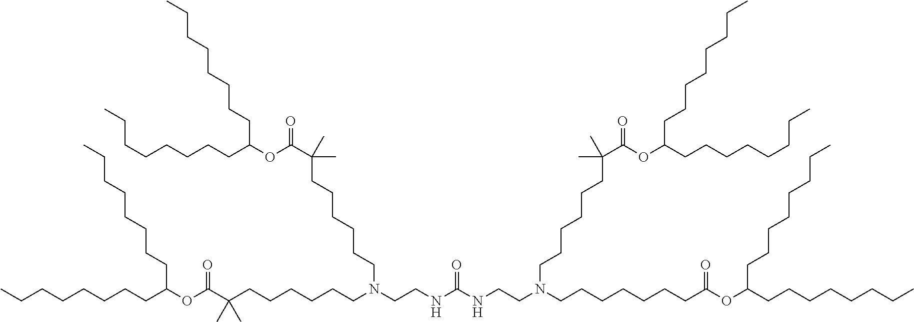 [SMILES: CCCCCCCCC(CCCCCCCC)OC(=O)CCCCCCCN(CCCCCCC(C)(C)C(=O)OC(CCCCCCCC)CCCCCCCC)CCNC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9-(8-(heptadecan-9- yloxy)-7,7-dimethyl- 8-oxooctyl)-17-(8- (heptadecan-9- yloxy)-8-oxooctyl)- 2,2,24,24- tetramethyl-13-oxo- 9,12,14,17- tetraaza- pentacosanedioate |
| A67 | 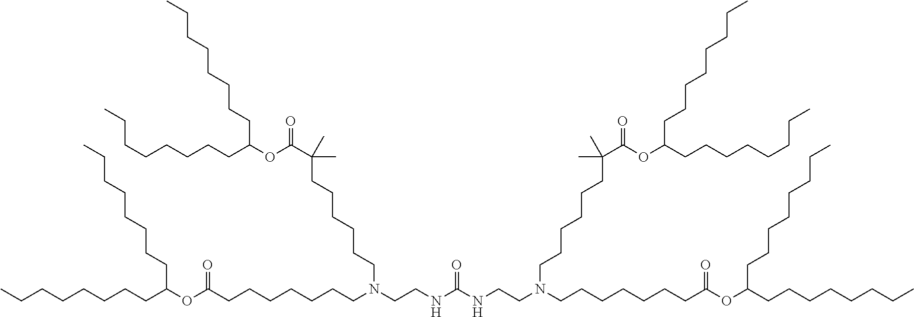 [SMILES: CCCCCCCCC(CCCCCCCC)OC(=O)CCCCCCCN(CCCCCCC(C)(C)C(=O)OC(CCCCCCCC)CCCCCCCC)CCNC(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,17-bis(8- (heptadecan-9- yloxy)-8-oxooctyl)- 2,2,24,24- tetramethyl-13-oxo- 9,12,14,17- tetraaza- pentacosanedioate |
| A68 | 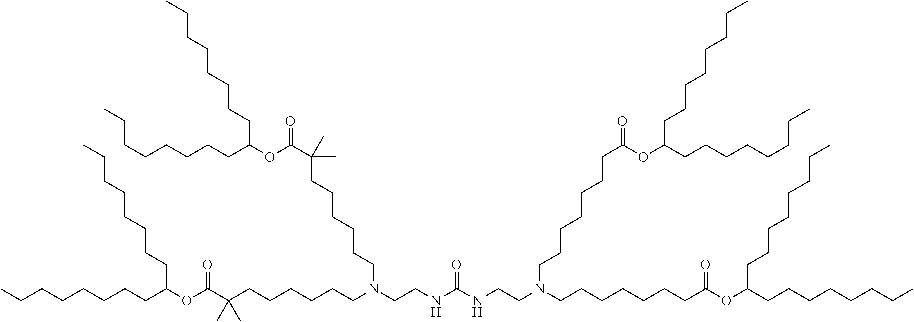 [SMILES: CCCCCCCCC(CCCCCCCC)OC(=O)CCCCCCCN(CCCCCCCC(=O)OC(CCCCCCCC)CCCCCCCC)CCNC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9-(8-(heptadecan-9- yloxy)-7,7-dimethyl- 8-oxooctyl)-17-(8- (heptadecan-9- yloxy)-8-oxooctyl)- 2,2-dimethyl-13-oxo- 9,12,14,17- tetraaza- pentacosanedioate |
| A69 | 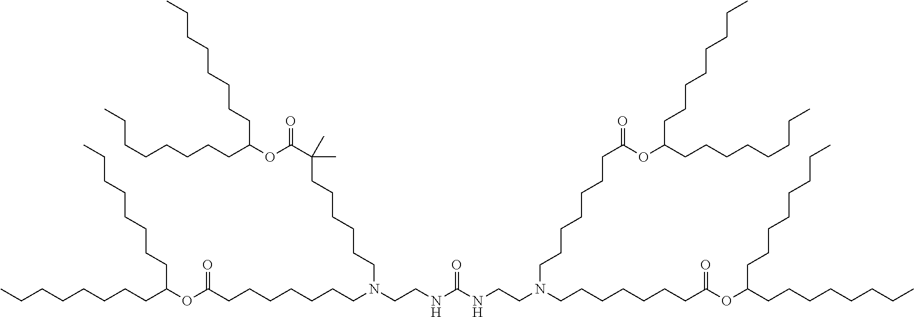 [SMILES: CCCCCCCCC(CCCCCCCC)OC(=O)CCCCCCCN(CCCCCCCC(=O)OC(CCCCCCCC)CCCCCCCC)CCNC(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,17-bis(8- (heptadecan-9- yloxy)-8-oxooctyl)- 2,2-dimethyl-13-oxo- 9,12,14,17- tetraaza- pentacosanedioate |
| A70 | 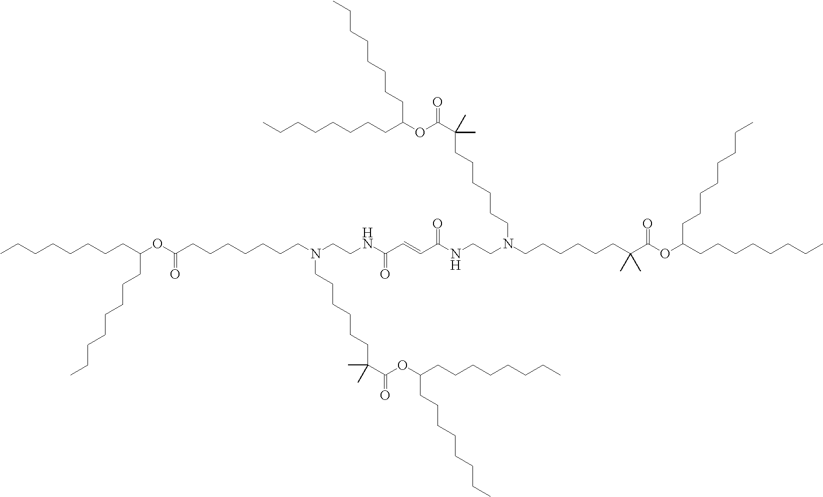 [SMILES: CCCCCCCCC(CCCCCCCC)OC(=O)CCCCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) (E)-9-(8- (heptadecan-9- yloxy)-7,7-dimethyl- 8-oxooctyl)-20-(8- (heptadecan-9- yloxy)-8-oxooctyl)- 2,2,27,27- tetramethyl-13,16- dioxo-9,12,17,20- tetraazaoctacos-14- enedioate |
| A71 | 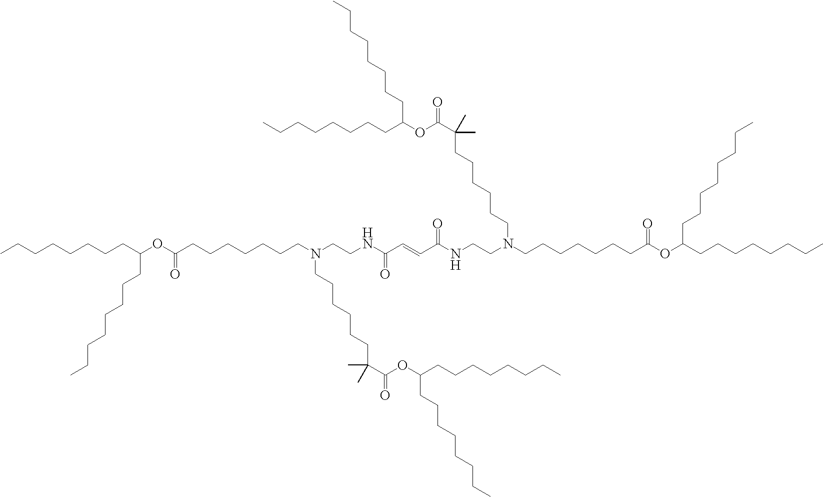 [SMILES: CCCCCCCCC(CCCCCCCC)OC(=O)CCCCCCCN(CCCCCCC(C)(C)C(=O)OC(CCCCCCCC)CCCCCCCC)CCNC(=O)/C=C/C(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) (E)-9,20-bis(8- (heptadecan-9- yloxy)-8-oxooctyl)- 2,2,27,27- tetramethyl-13,16- dioxo-9,12,17,20- tetraazaoctacos-14- enedioate |
| A72 | 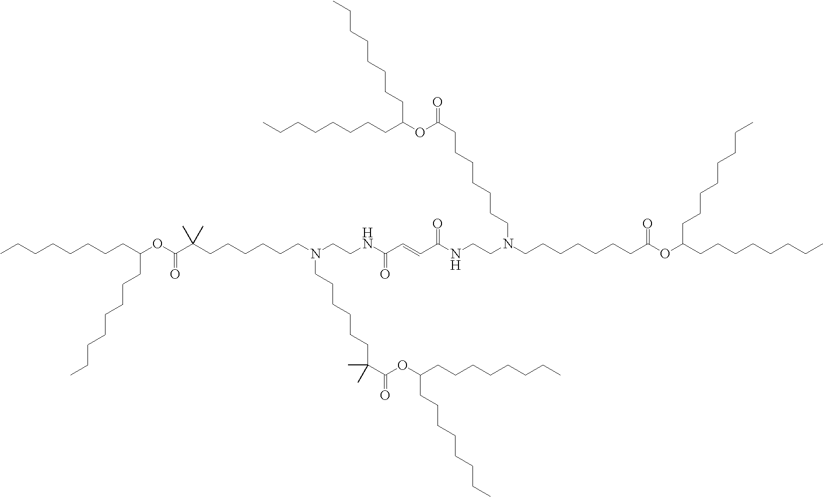 [SMILES: CCCCCCCCC(CCCCCCCC)OC(=O)CCCCCCCN(CCCCCCCC(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) (E)-9-(8- (heptadecan-9- yloxy)-7,7-dimethyl- 8-oxooctyl)-20-(8- (heptadecan-9- yloxy)-8-oxooctyl)- 2,2-dimethyl-13,16- dioxo-9,12,17,20- tetraazaoctacos-14- enedioate |
| A73 | 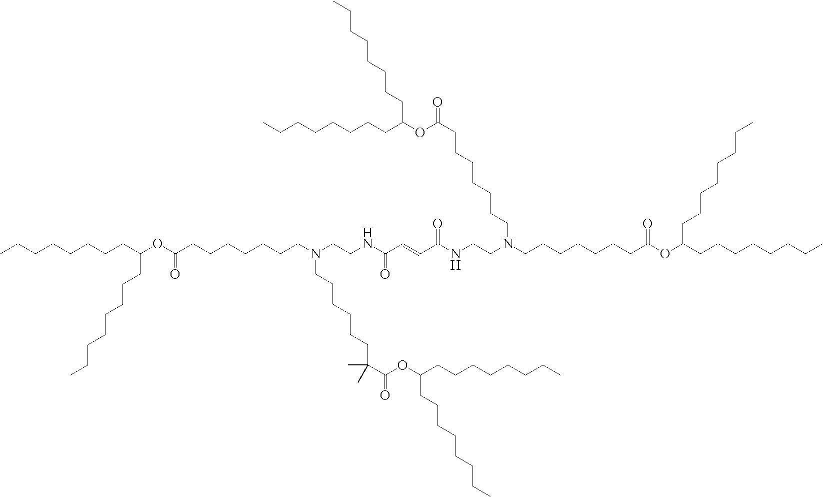 [SMILES: CCCCCCCCC(CCCCCCCC)OC(=O)CCCCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) (E)-9,20-bis(8- (heptadecan-9- yloxy)-8-oxooctyl)- 2,2-dimethyl-13,16- dioxo-9,12,17,20- tetraazaoctacos-14- enedioate |
| A74 | 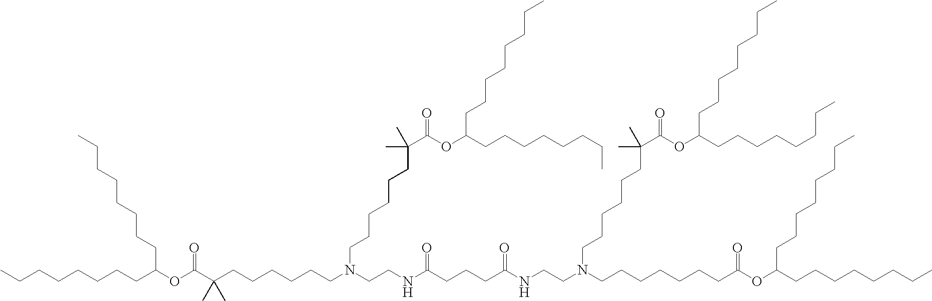 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCCC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9-(8-(heptadecan-9- yloxy)-7,7-dimethyl- 8-oxooctyl)-21-(8- (heptadecan-9- yloxy)-8-oxooctyl)- 2,2,28,28- tetramethyl-13,17- dioxo-9,12,18,21- tetraaza- nonacosanedioate |
| A75 | 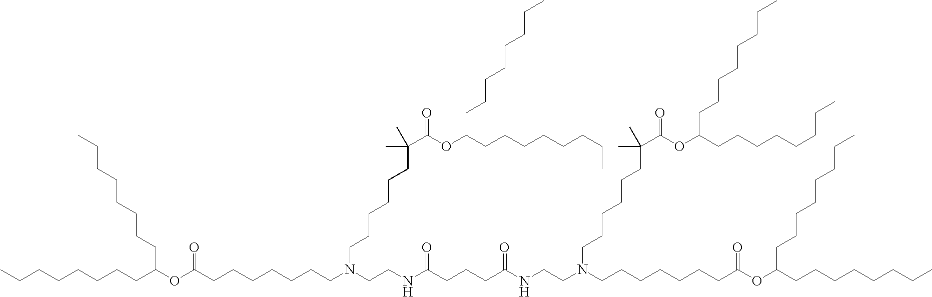 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCCC(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,21-bis(8- (heptadecan-9- yloxy)-8-oxooctyl)- 2,2,28,28- tetramethyl-13,17- dioxo-9,12,18,21- tetraaza- nonacosanedioate |
| A76 | 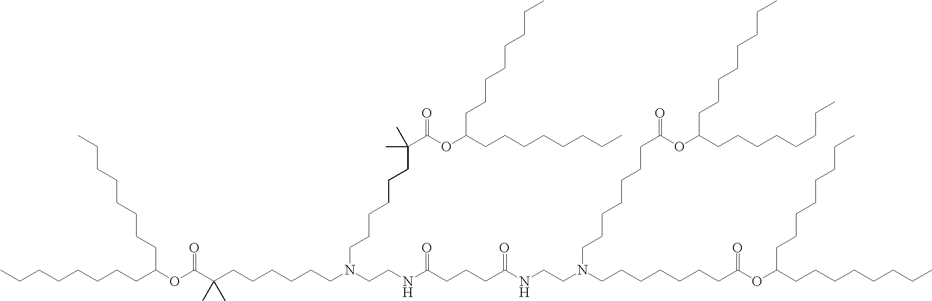 [SMILES: CCCCCCCCC(CCCCCCCC)OC(=O)CCCCCCCN(CCCCCCCC(=O)OC(CCCCCCCC)CCCCCCCC)CCNC(=O)CCCC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9-(8-(heptadecan-9- yloxy)-7,7-dimethyl- 8-oxooctyl)-21-(8- (heptadecan-9- yloxy)-8-oxooctyl)- 2,2-dimethyl-13,17- dioxo-9,12,18,21- tetraaza- nonacosanedioate |
| A77 | 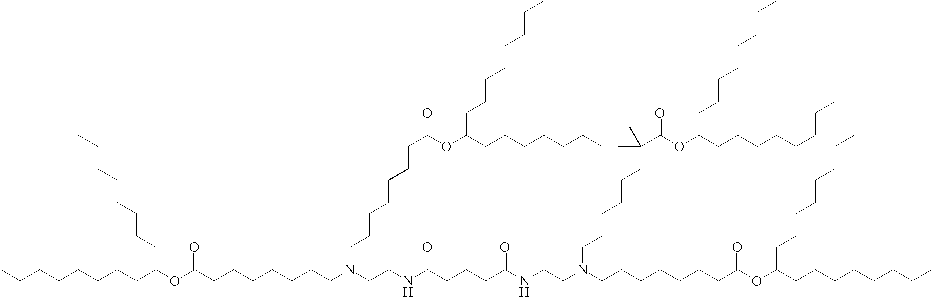 [SMILES: CCCCCCCCC(CCCCCCCC)OC(=O)CCCCCCCN(CCCCCCCC(=O)OC(CCCCCCCC)CCCCCCCC)CCNC(=O)CCCC(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,21-bis(8- (heptadecan-9- yloxy)-8-oxooctyl)- 2,2-dimethyl-13,17- dioxo-9,12,18,21- tetraaza- nonacosanedioate |
| A78 | 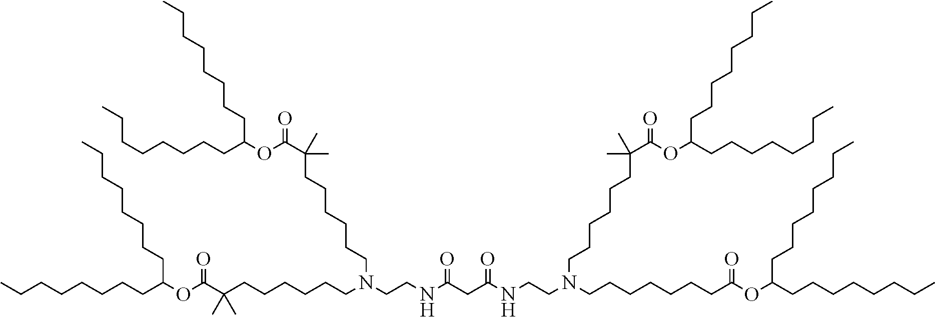 [SMILES: CCCCCCCCC(CCCCCCCC)OC(=O)CCCCCCCN(CCCCCCC(C)(C)C(=O)OC(CCCCCCCC)CCCCCCCC)CCNC(=O)CC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9-(8-(heptadecan-9- yloxy)-7,7-dimethyl- 8-oxooctyl)-19-(8- (heptadecan-9- yloxy)-8-oxooctyl)- 2,2,26,26- tetramethyl-13,15- dioxo-9,12,16,19- tetraaza- heptacosanedioate |
| A79 | 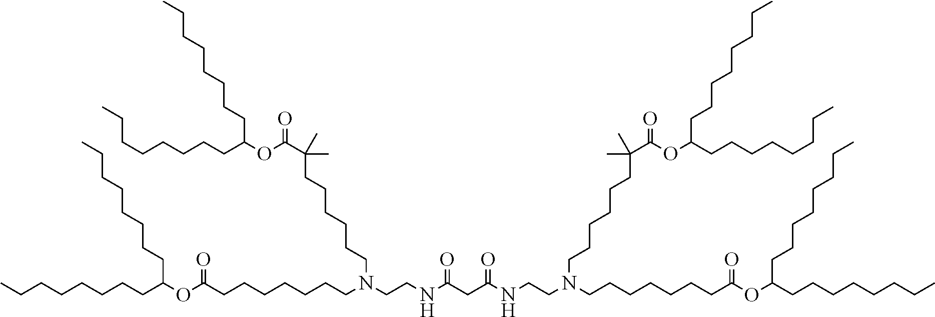 [SMILES: CCCCCCCCC(CCCCCCCC)OC(=O)CCCCCCCN(CCCCCCC(C)(C)C(=O)OC(CCCCCCCC)CCCCCCCC)CCNC(=O)CC(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,19-bis(8- (heptadecan-9- yloxy)-8-oxooctyl)- 2,2,26,26- tetramethyl-13,15- dioxo-9,12,16,19- tetraaza- heptacosanedioate |
| A80 | 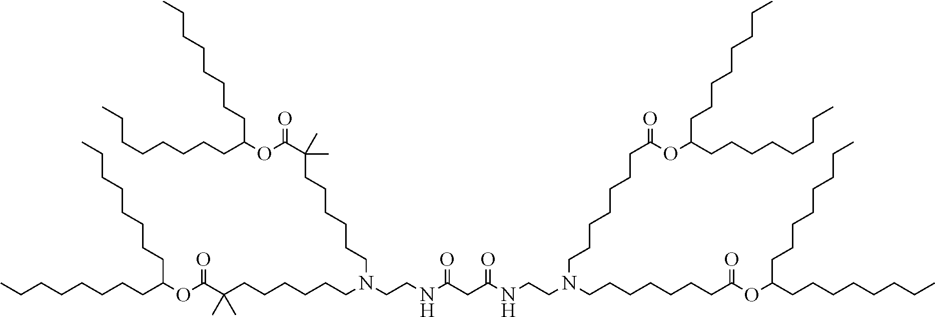 [SMILES: CCCCCCCCC(CCCCCCCC)OC(=O)CCCCCCCN(CCCCCCCC(=O)OC(CCCCCCCC)CCCCCCCC)CCNC(=O)CC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9-(8-(heptadecan-9- yloxy)-7,7-dimethyl- 8-oxooctyl)-19-(8- (heptadecan-9- yloxy)-8-oxooctyl)- 2,2-dimethyl-13,15- dioxo-9,12,16,19- tetraaza- heptacosanedioate |
| A81 | 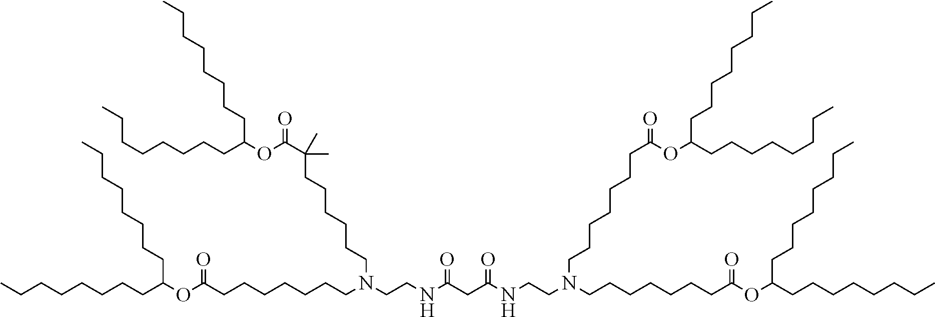 [SMILES: CCCCCCCCC(CCCCCCCC)OC(=O)CCCCCCCN(CCCCCCCC(=O)OC(CCCCCCCC)CCCCCCCC)CCNC(=O)CC(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,19-bis(8- (heptadecan-9- yloxy)-8-oxooctyl)- 2,2-dimethyl-13,15- dioxo-9,12,16,19- tetraaza- heptacosanedioate |
| A82 | 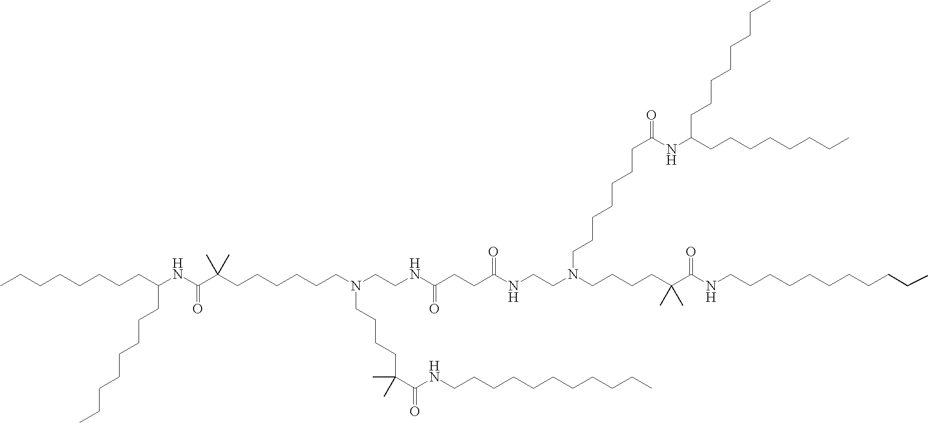 [SMILES: CCCCCCCCCCCNC(=O)C(C)(C)CCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)NCCCCCCCCCCC] | N¹-(2-((5,5-dimethyl- 6-oxo-6- (undecylamino)hexyl) (8-(heptadecan-9- ylamino)-7,7- dimethyl-8- oxooctyl)amino)ethyl)- N⁴-(2-((5,5- dimethyl-6-oxo-6- (undecylamino)hexyl) (8-(heptadecan-9- ylamino)-8- oxooctyl)amino)ethyl) succinamide |
| A83 | 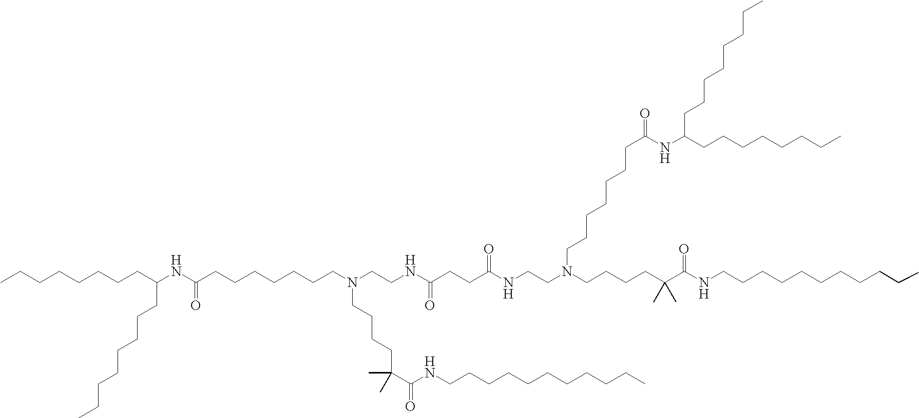 [SMILES: CCCCCCCCCCCNC(=O)C(C)(C)CCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)NCCCCCCCCCCC] | N¹,N⁴-bis(2-((5,5- dimethyl-6-oxo-6- (undecylamino)hexyl) (8-(heptadecan-9- ylamino)-8- oxooctyl)amino)ethyl) succinamide |
| A84 | 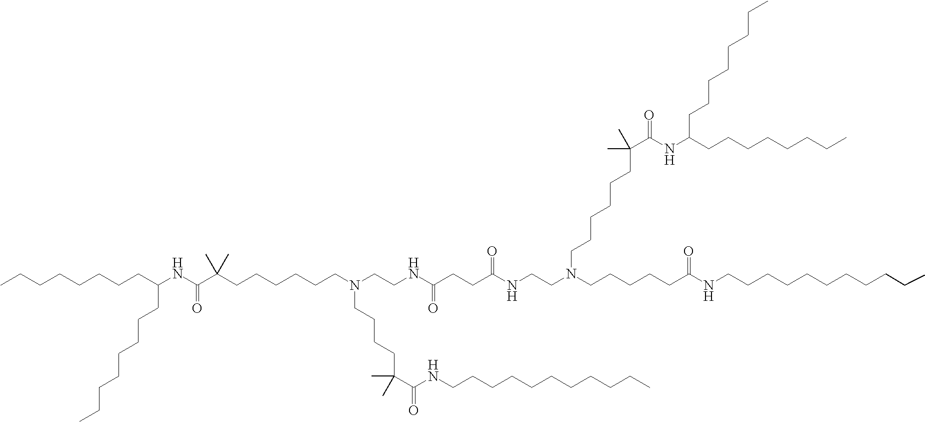 [SMILES: CCCCCCCCCCCNC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)NCCCCCCCCCCC] | N¹-(2-((5,5-dimethyl- 6-oxo-6- (undecylamino)hexyl) (8-(heptadecan-9- ylamino)-7,7- dimethyl-8- oxooctyl)amino)ethyl)- N⁴-(2-((8- (heptadecan-9- ylamino)-7,7- dimethyl-8- oxooctyl)(6-oxo-6- (undecylamino)hexyl) amino)ethyl) succinamide |
| A85 | 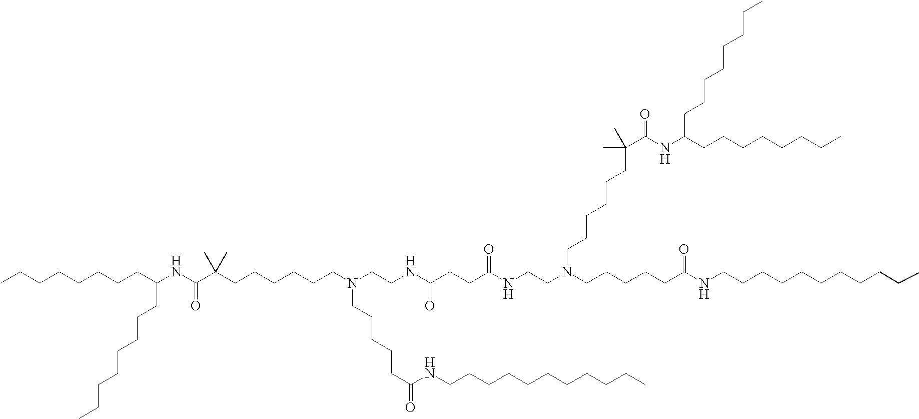 [SMILES: CCCCCCCCCCCNC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)NCCCCCCCCCCC] | N¹,N⁴-bis(2-((8- (heptadecan-9- ylamino)-7,7- dimethyl-8- oxooctyl)(6-oxo-6- (undecylamino)hexyl) amino)ethyl) succinamide |
| A86 | 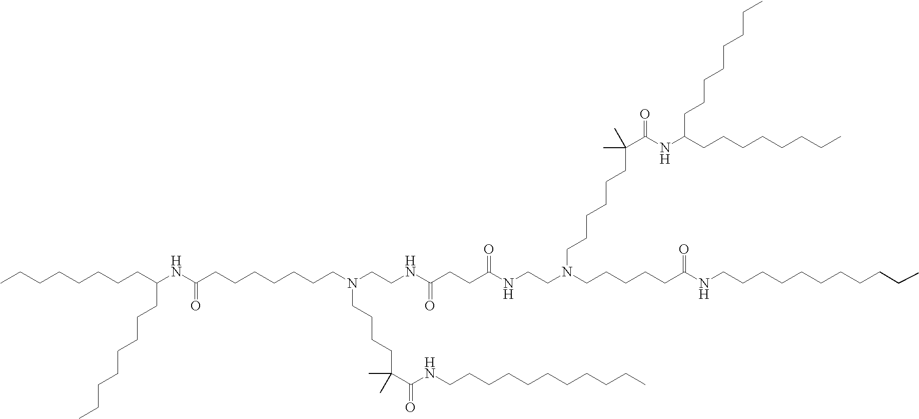 [SMILES: CCCCCCCCCCCNC(=O)CCCCCN(CCCCCCC(C)(C)C(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)NCCCCCCCCCCC] | N¹-(2-((5,5-dimethyl- 6-oxo-6- (undecylamino)hexyl) (8-(heptadecan-9- ylamino)-8- oxooctyl)amino)ethyl)- N⁴-(2-((8- (heptadecan-9- ylamino)-7,7- dimethyl-8- oxooctyl)(6-oxo-6- (undecylamino)hexyl) amino)ethyl) succinamide |
| A87 | 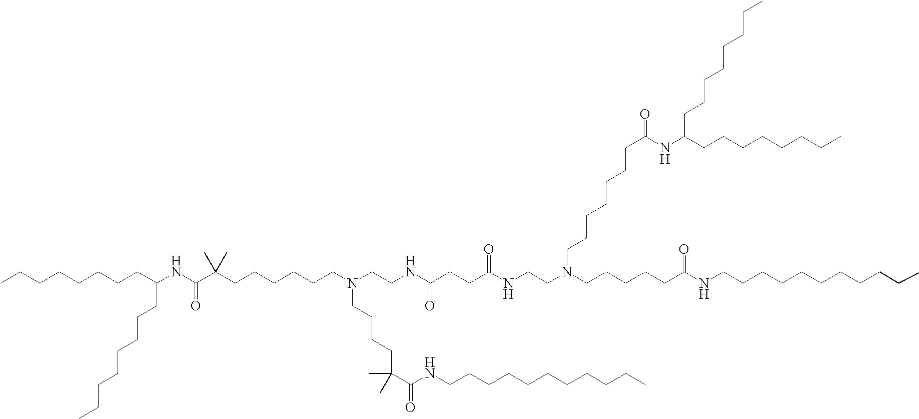 [SMILES: CCCCCCCCCCCNC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)NCCCCCCCCCCC] | N¹-(2-((5,5-dimethyl- 6-oxo-6- (undecylamino)hexyl) (8-(heptadecan-9- ylamino)-7,7- dimethyl-8- oxooctyl)amino)ethyl)- N⁴-(2-((8- (heptadecan-9- ylamino)-8- oxooctyl)(6-oxo-6- (undecylamino)hexyl) amino)ethyl) succinamide |
| A88 | 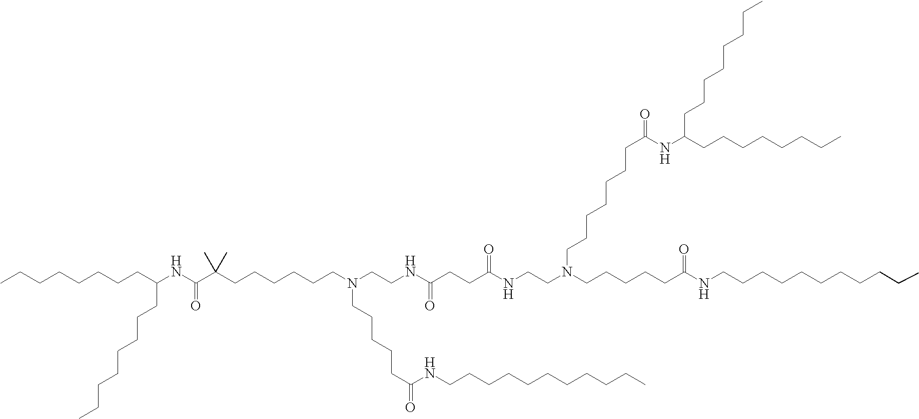 [SMILES: CCCCCCCCCCCNC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)NCCCCCCCCCCC] | N¹-(2-((8- (heptadecan-9- ylamino)-7,7- dimethyl-8- oxooctyl)(6-oxo-6- (undecylamino)hexyl) amino)ethyl)-N⁴-(2- ((8-(heptadecan-9- ylamino)-8- oxooctyl)(6-oxo-6- (undecylamino)hexyl) amino)ethyl) succinamide |
| A89 | 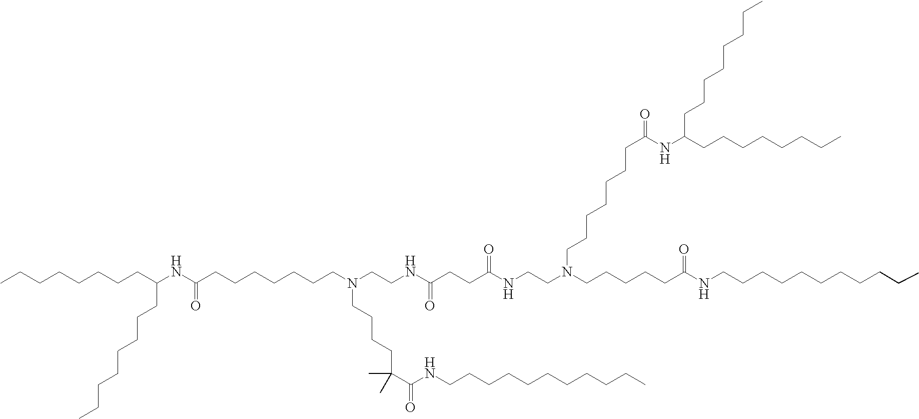 [SMILES: CCCCCCCCCCCNC(=O)CCCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)NCCCCCCCCCCC] | N¹-(2-((5,5-dimethyl- 6-oxo-6- (undecylamino)hexyl) (8-(heptadecan-9- ylamino)-8- oxooctyl)amino)ethyl)- N⁴-(2-((8- (heptadecan-9- ylamino)-8- oxooctyl)(6-oxo-6- (undecylamino)hexyl) amino)ethyl) succinamide |
| A90 | 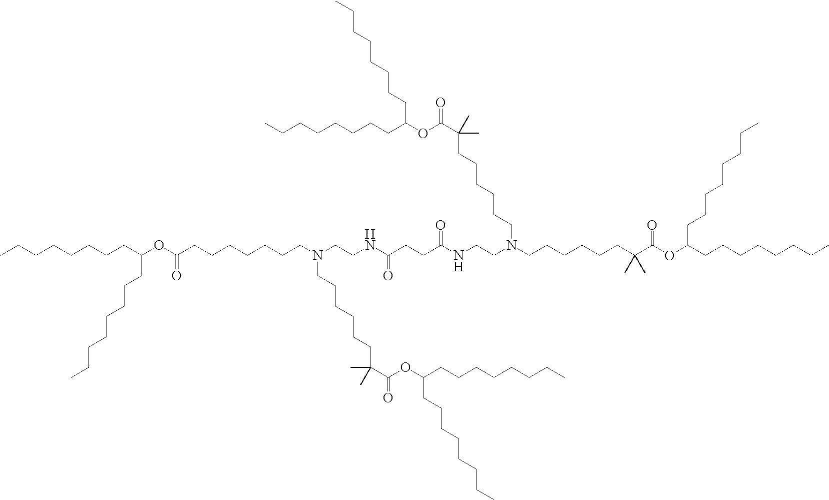 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9-(8-(heptadecan-9- yloxy)-7,7-dimethyl- 8-oxooctyl)-20-(8- (heptadecan-9- yloxy)-8-oxooctyl)- 2,2,27,27- tetramethyl-13,16- dioxo-9,12,17,20- tetraaza- octacosanedioate |
| A91 | 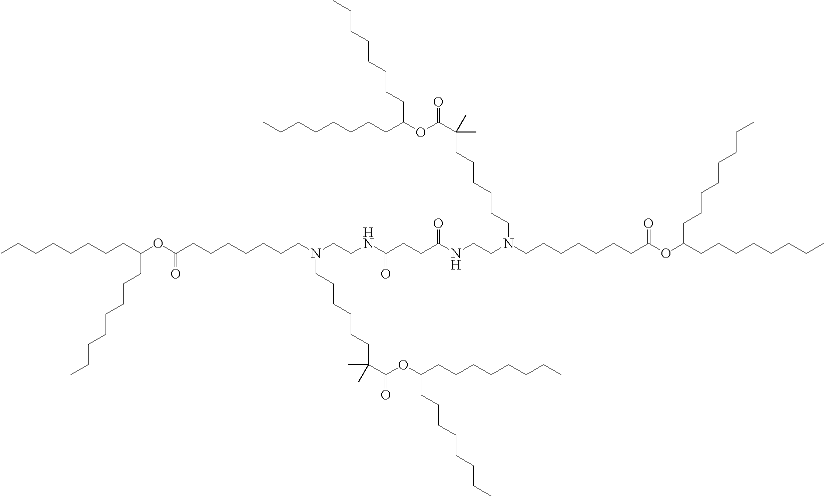 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,20-bis(8- (heptadecan-9- yloxy)-8-oxooctyl)- 2,2,27,27- tetramethyl-13,16- dioxo-9,12,17,20- tetraaza- octacosanedioate |
| A92 | 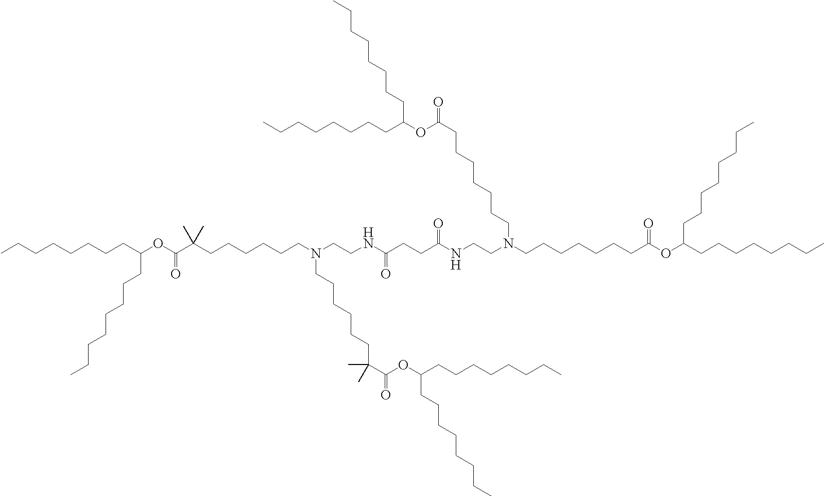 [SMILES: CCCCCCCCC(CCCCCCCC)OC(=O)CCCCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCCC(C)(C)C(=O)OC(CCCCCCCC)CCCCCCCC] | di(heptadecan-9-yl) 9-(8-(heptadecan-9- yloxy)-7,7-dimethyl- 8-oxooctyl)-20-(8- (heptadecan-9- yloxy)-8-oxooctyl)- 2,2-dimethyl-13,16- dioxo-9,12,17,20- tetraaza- octacosanedioate |
| A93 | 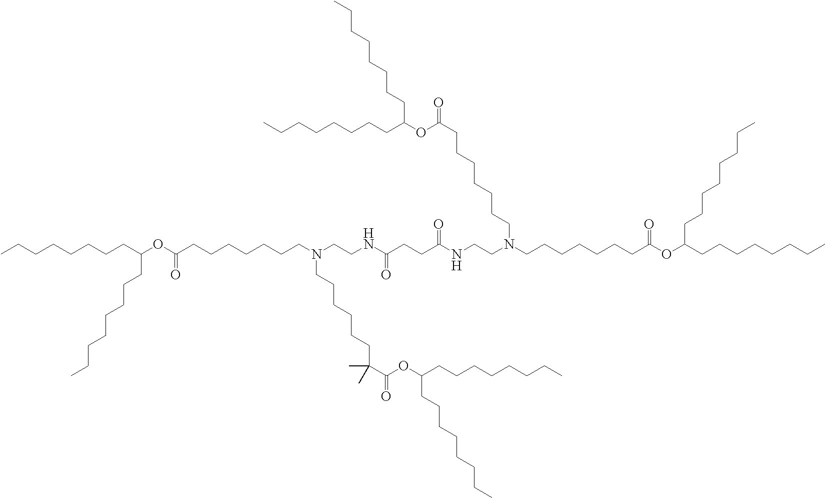 [SMILES: CCCCCCCCC(CCCCCCCC)OC(=O)CCCCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCCC(C)(C)C(=O)OC(CCCCCCCC)CCCCCCCC] | di(heptadecan-9-yl) 9,20-bis(8- (heptadecan-9- yloxy)-8-oxooctyl)- 2,2-dimethyl-13,16- dioxo-9,12,17,20- tetraaza- octacosanedioate |
| A94 | 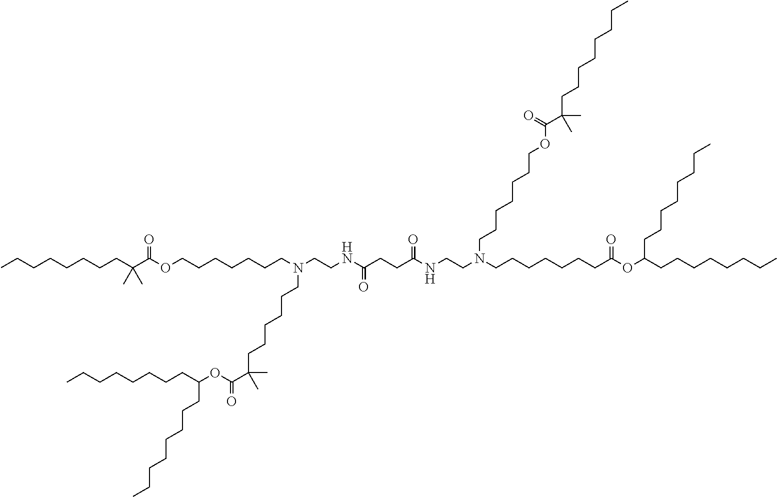 [SMILES: CCCCCCCCC(CCCCCCCC)OC(=O)CCCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCC(C)(C)C(=O)OC(CCCCCCCC)CCCCCCCC] | di(heptadecan-9-yl) 9,20-bis(7-((2,2- dimethyldecanoyl) oxy)heptyl)-2,2- dimethyl-13,16- dioxo-9,12,17,20- tetraaza- octacosanedioate |
| A95 | 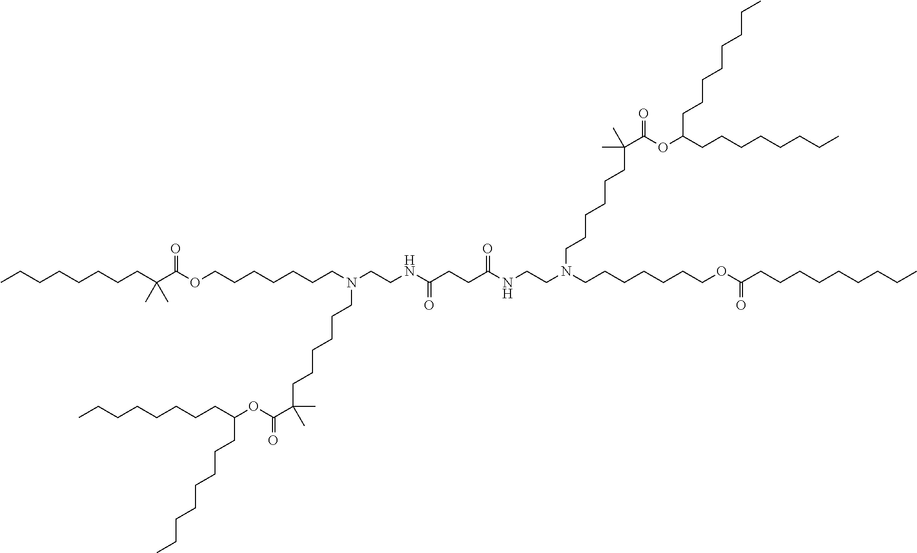 [SMILES: CCCCCCCCCC(=O)OCCCCCCCN(CCCCCCC(C)(C)C(=O)OC(CCCCCCCC)CCCCCCCC)CCNC(=O)CCC(=O)NCCN(CCCCCCCOC(=O)C(C)(C)CCCCCCCC)CCCCCCC(C)(C)C(=O)OC(CCCCCCCC)CCCCCCCC] | di(heptadecan-9-yl) 9-(7- (decanoyloxy)heptyl)- 20-(7-((2,2- dimethyldecanoyl) oxy)heptyl)-2,2,27,27- tetramethyl-13,16- dioxo-9,12,17,20- tetraaza- octacosanedioate |
| A96 | 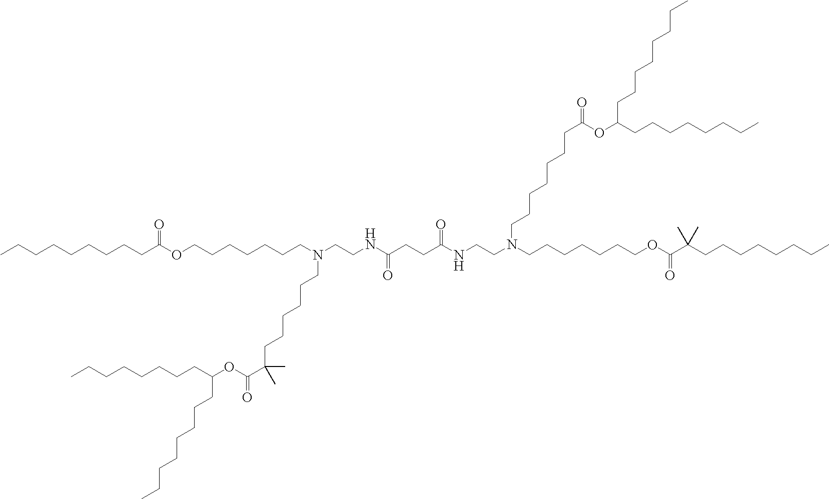 [SMILES: CCCCCCCCCC(=O)OCCCCCCCN(CCCCCCC(C)(C)C(=O)OC(CCCCCCCC)CCCCCCCC)CCNC(=O)CCC(=O)NCCN(CCCCCCCOC(=O)C(C)(C)CCCCCCCC)CCCCCCCC(=O)OC(CCCCCCCC)CCCCCCCC] | di(heptadecan-9-yl) 9-(7- (decanoyloxy)heptyl)- 20-(7-((2,2- dimethyldecanoyl) oxy)heptyl)-2,2- dimethyl-13,16- dioxo-9,12,17,20- tetraaza- octacosanedioate |
| A97 | 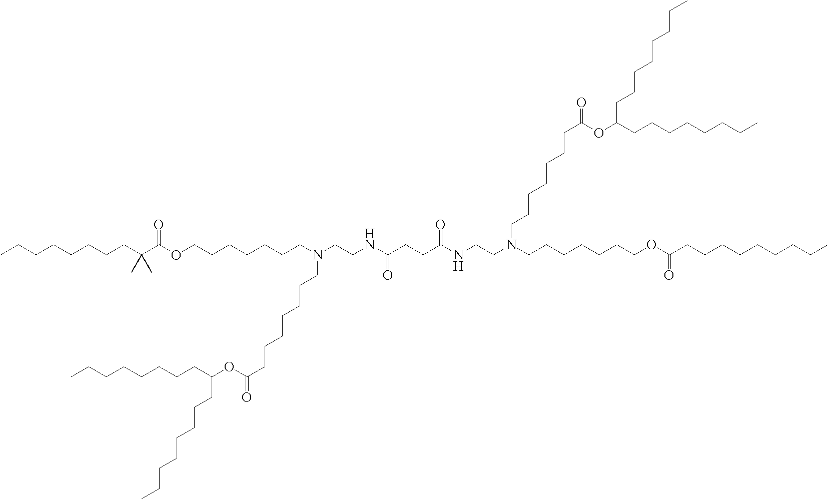 [SMILES: CCCCCCCCCC(=O)OCCCCCCCN(CCCCCCCC(=O)OC(CCCCCCCC)CCCCCCCC)CCNC(=O)CCC(=O)NCCN(CCCCCCCOC(=O)C(C)(C)CCCCCCCC)CCCCCCCC(=O)OC(CCCCCCCC)CCCCCCCC] | di(heptadecan-9-yl) 9-(7- (decanoyloxy)heptyl)- 20-(7-((2,2- dimethyldecanoyl) oxy)heptyl)-13,16- dioxo-9,12,17,20- tetraaza- octacosanedioate |
| A98 | 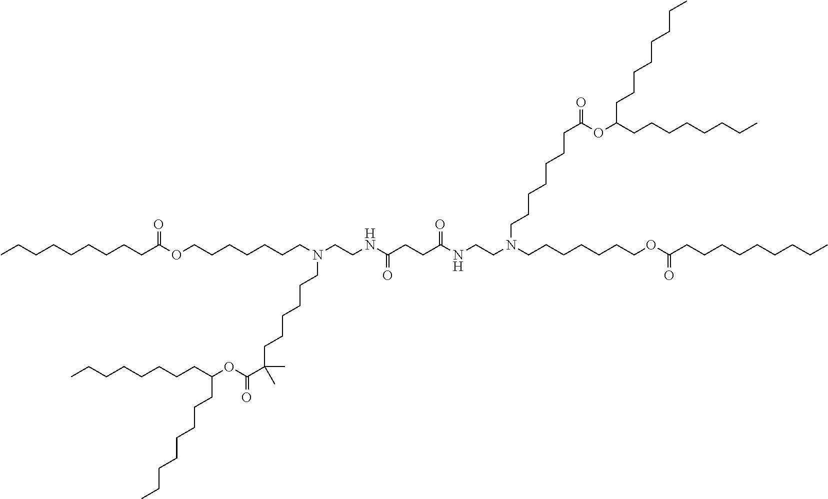 [SMILES: CCCCCCCCCC(=O)OCCCCCCCN(CCCCCCCC(=O)OC(CCCCCCCC)CCCCCCCC)CCNC(=O)CCC(=O)NCCN(CCCCCCCOC(=O)CCCCCCCCC)CCCCCCC(C)(C)C(=O)OC(CCCCCCCC)CCCCCCCC] | di(heptadecan-9-yl) 9,20-bis(7- (decanoyloxy)heptyl)- 2,2-dimethyl-13,16- dioxo-9,12,17,20- tetraaza- octacosanedioate |
| A99 | 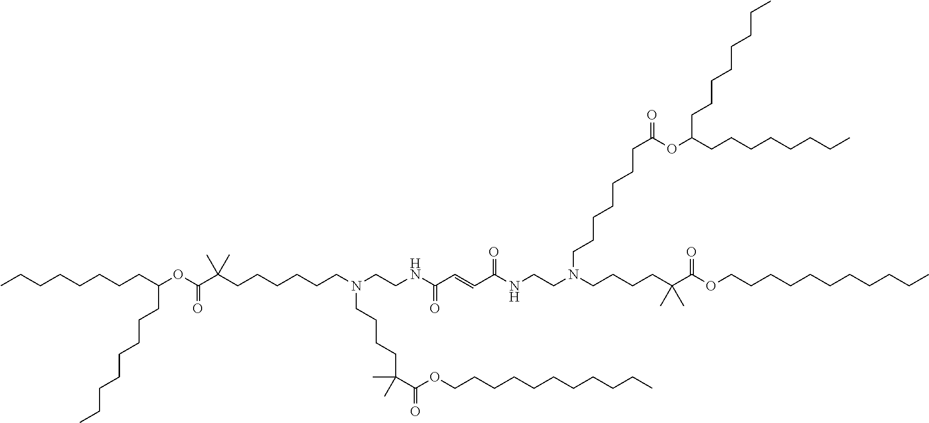 [SMILES: CCCCCCCCCCCOC(=O)C(C)(C)CCCCN(CCCCCCCC(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) (E)-9,20-bis(5,5- dimethyl-6-oxo-6- (undecyloxy)hexyl)- 2,2-dimethyl-13,16- dioxo-9,12,17,20- tetraazaoctacos-14- enedioate |
| A100 | 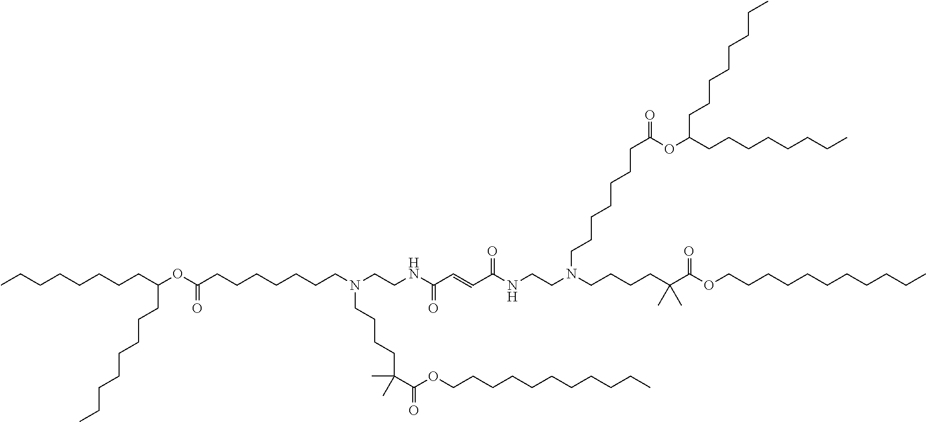 [SMILES: CCCCCCCCCCCOC(=O)C(C)(C)CCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCC(C)(C)C(=O)OCCCCCCCCCCC] | di(heptadecan-9-yl) (E)-9,20-bis(5,5- dimethyl-6-oxo-6- (undecyloxy)hexyl)- 13,16-dioxo- 9,12,17,20- tetraazaoctacos-14- enedioate |
| A101 | 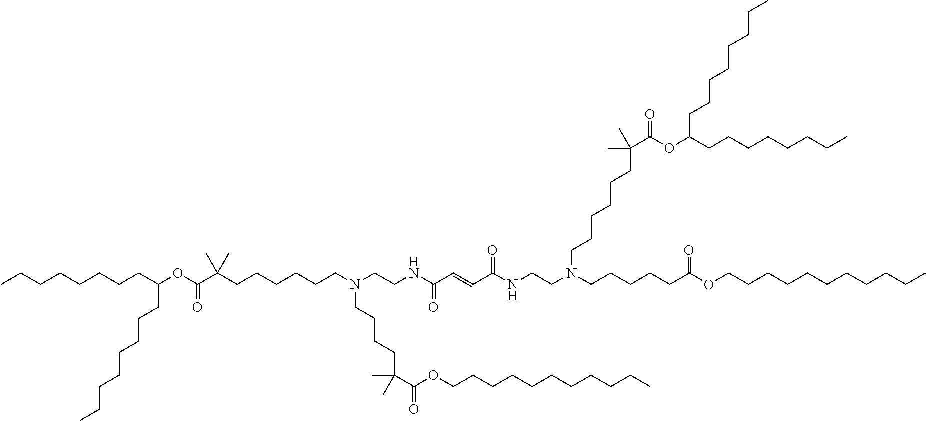 [SMILES: CCCCCCCCCCCOC(=O)CCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) (E)-9-(5,5-dimethyl- 6-oxo-6- (undecyloxy)hexyl)- 2,2,27,27- tetramethyl-13,16- dioxo-20-(6-oxo-6- (undecyloxy)hexyl)- 9,12,17,20- tetraazaoctacos-14- enedioate |
| A102 | 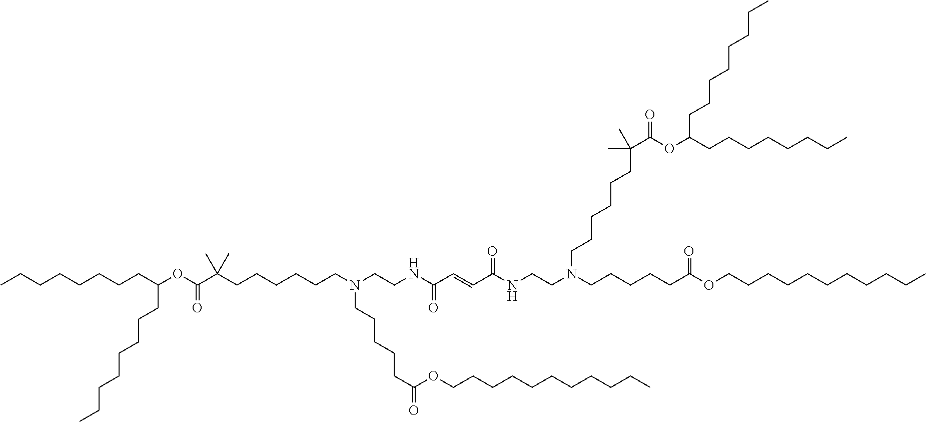 [SMILES: CCCCCCCCCCCOC(=O)CCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCC(=O)OCCCCCCCCCCC] | di(heptadecan-9-yl) (E)-2,2,27,27- tetramethyl-13,16- dioxo-9,20-bis(6- oxo-6- (undecyloxy)hexyl)- 9,12,17,20- tetraazaoctacos-14- enedioate |
| A103 | 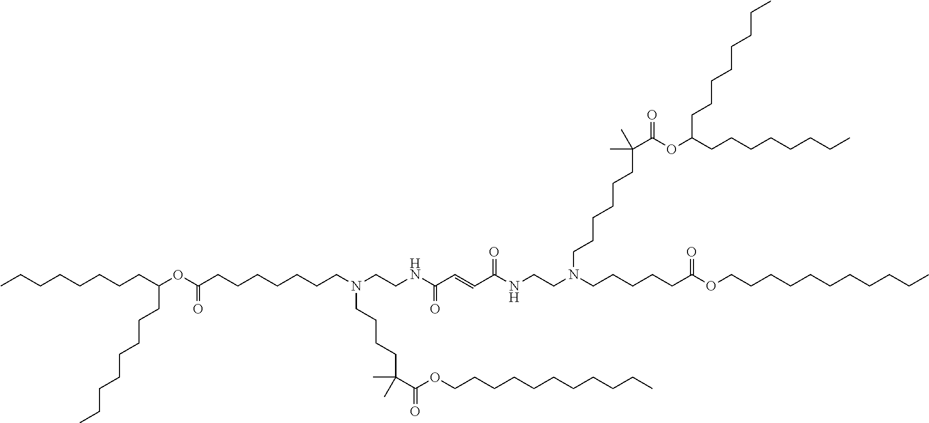 [SMILES: CCCCCCCCCCCOC(=O)CCCCCN(CCCCCCC(C)(C)C(=O)OC(CCCCCCCC)CCCCCCCC)CCNC(=O)/C=C/C(=O)NCCN(CCCCCCCC(=O)OC(CCCCCCCC)CCCCCCCC)CCCCC(C)(C)C(=O)OCCCCCCCCCCC] | di(heptadecan-9-yl) (E)-20-(5,5-dimethyl- 6-oxo-6- (undecyloxy)hexyl)- 2,2-dimethyl-13,16- dioxo-9-(6-oxo-6- (undecyloxy)hexyl)- 9,12,17,20- tetraazaoctacos-14- enedioate |
| A104 | 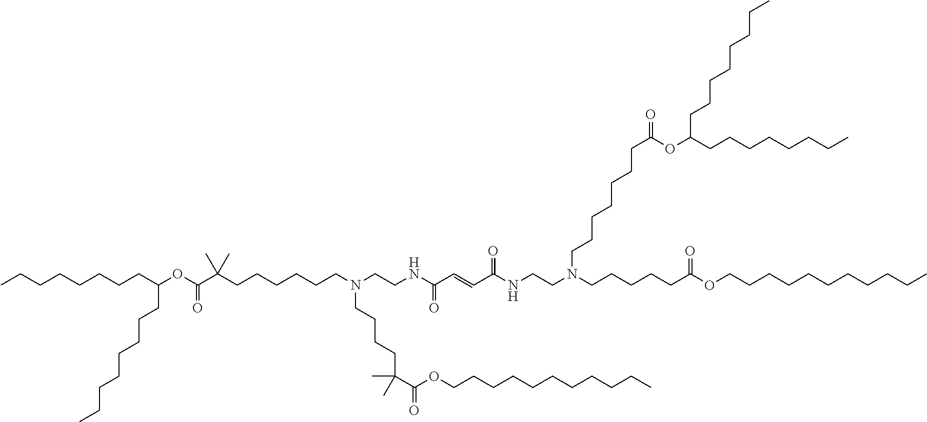 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) (E)-9-(5,5-dimethyl- 6-oxo-6- (undecyloxy)hexyl)- 2,2-dimethyl-13,16- dioxo-20-(6-oxo-6- (undecyloxy)hexyl)- 9,12,17,20- tetraazaoctacos-14- enedioate |
| A105 | 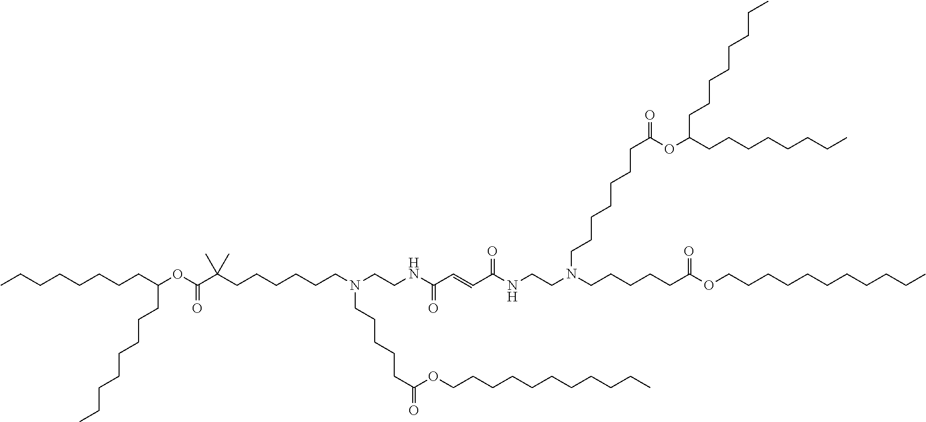 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCC(=O)OCCCCCCCCCCC] | di(heptadecan-9-yl) (E)-2,2-dimethyl- 13,16-dioxo-9,20- bis(6-oxo-6- (undecyloxy)hexyl)- 9,12,17,20- tetraazaoctacos-14- enedioate |
| A106 | 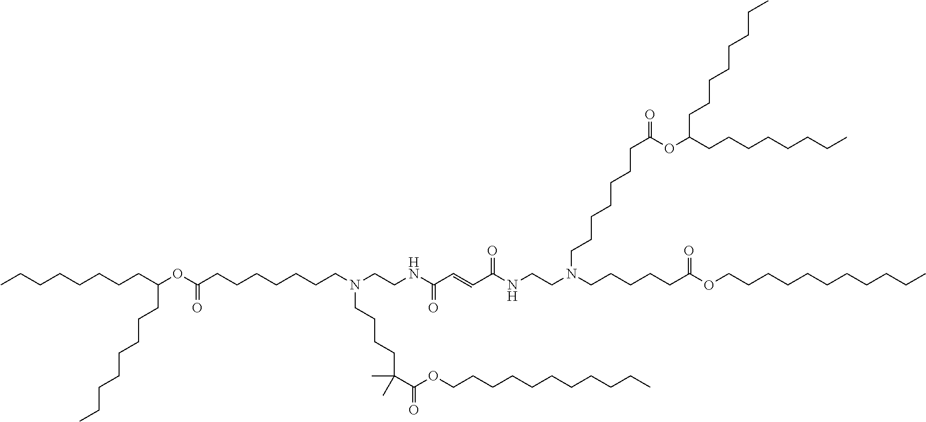 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCC(C)(C)C(=O)OCCCCCCCCCCC] | di(heptadecan-9-yl) (E)-9-(5,5-dimethyl- 6-oxo-6- (undecyloxy)hexyl)- 13,16-dioxo-20-(6- oxo-6- (undecyloxy)hexyl)- 9,12,17,20- tetraazaoctacos-14- enedioate |
| B1 | 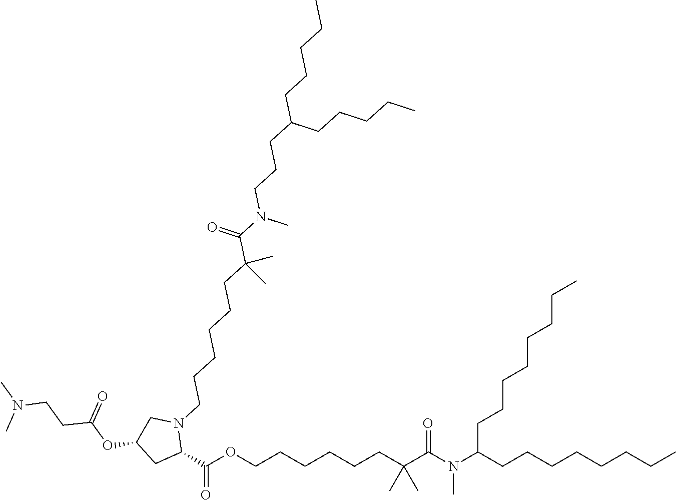 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCC(C)(C)C(=O)N(C)CCCC(CCCCC)CCCCC] | 8-(heptadecan-9- yl(methyl)amino)- 7,7-dimethyl-8- oxooctyl (2S,4S)-1- (7,7-dimethyl-8- (methyl(4- pentylnonyl)amino)- 8-oxooctyl)-4-((3- (dimethylamino) propanoyl)oxy) pyrrolidine- 2-carboxylate |
| B2 | 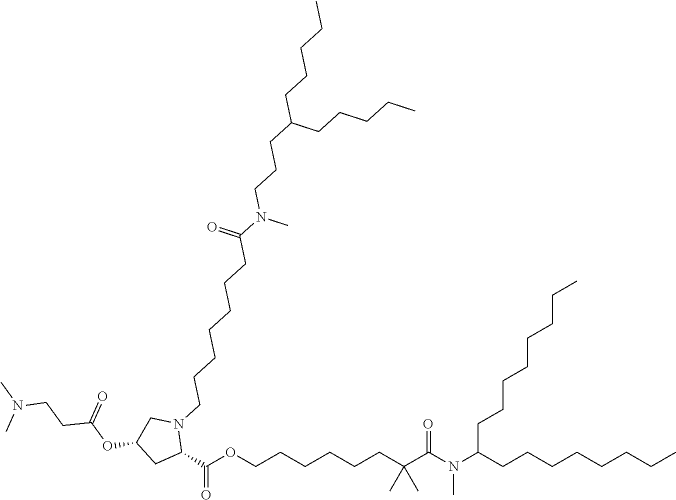 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCCC(=O)N(C)CCCC(CCCCC)CCCCC] | 8-(heptadecan-9- yl(methyl)amino)- 7,7-dimethyl-8- oxooctyl (2S,4S)-4- ((3- (dimethylamino) propanoyl)oxy)-1-(8- (methyl(4- pentylnonyl)amino)- 8- oxooctyl)pyrrolidine- 2-carboxylate |
| B3 | 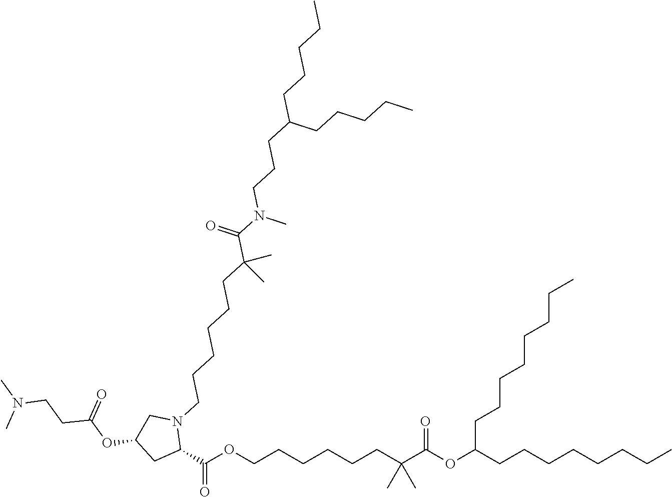 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)N(C)CCCC(CCCCC)CCCCC] | 8-(heptadecan-9- yloxy)-7,7-dimethyl- 8-oxooctyl (2S,4S)- 1-(7,7-dimethyl-8- (methyl(4- pentylnonyl)amino)- 8-oxooctyl)-4-((3- (dimethylamino) propanoyl)oxy) pyrrolidine- 2-carboxylate |
| B4 | 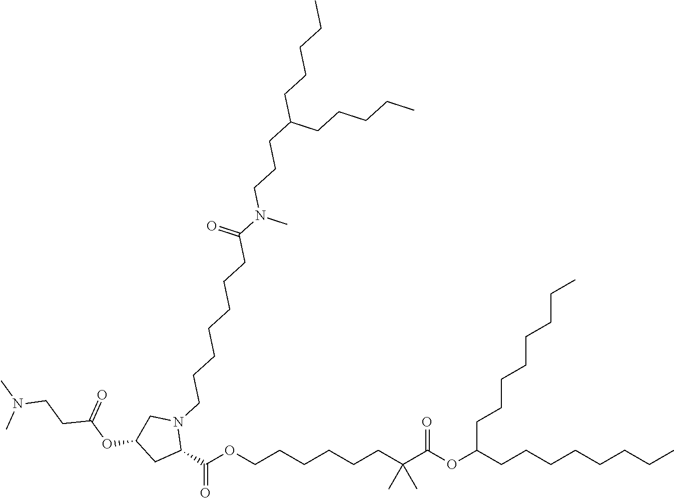 [SMILES: CCCCCCCCC(CCCCCCCC)OC(=O)C(C)(C)CCCCCCOC(=O)[C@@H]1C[C@H](OC(=O)CCN(C)C)CN1CCCCCCCC(=O)N(C)CCCC(CCCCC)CCCCC] | 8-(heptadecan-9- yloxy)-7,7-dimethyl- 8-oxooctyl (2S,4S)- 4-((3- (dimethylamino) propanoyl)oxy)-1-(8- (methyl(4- pentylnonyl)amino)- 8- oxooctyl)pyrrolidine- 2-carboxylate |
| B5 | 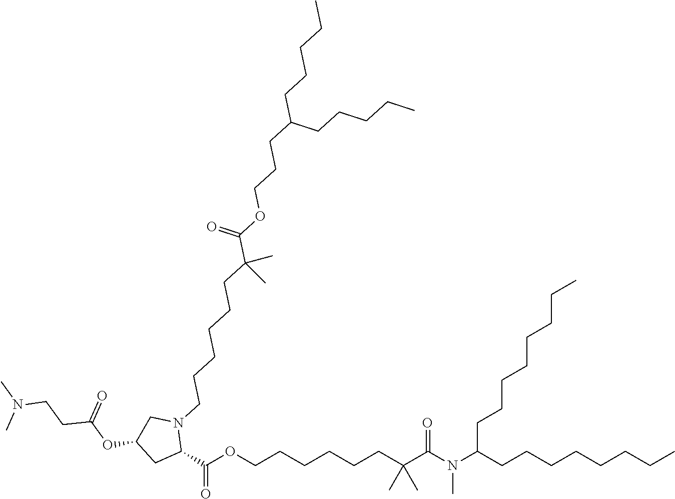 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCC(C)(C)C(=O)OCCCC(CCCCC)CCCCC] | 8-(heptadecan-9- yl(methyl)amino)- 7,7-dimethyl-8- oxooctyl (2S,4S)-1- (7,7-dimethyl-8-oxo- 8-((4- pentylnonyl)oxy) octyl)-4-((3- (dimethylamino) propanoyl)oxy) pyrrolidine- 2-carboxylate |
| B6 | 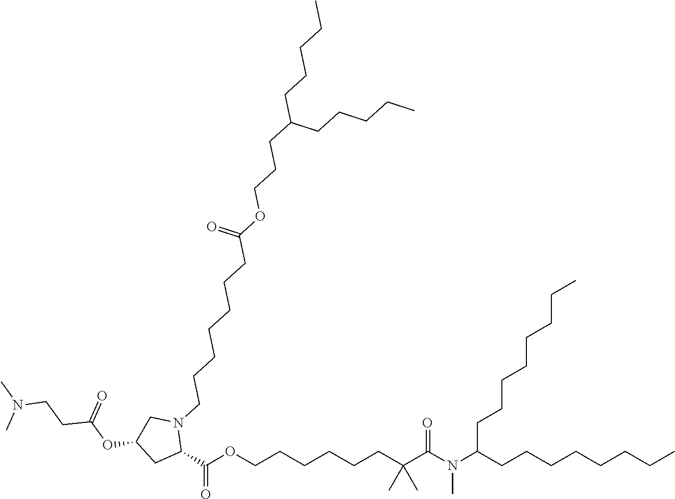 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCCC(=O)OCCCC(CCCCC)CCCCC] | 8-(heptadecan-9- yl(methyl)amino)- 7,7-dimethyl-8- oxooctyl (2S,4S)-4- ((3- (dimethylamino) propanoyl)oxy)- 1-(8-oxo- 8-((4- pentylnonyl)oxy) octyl)pyrrolidine-2- carboxylate |
| B7 | 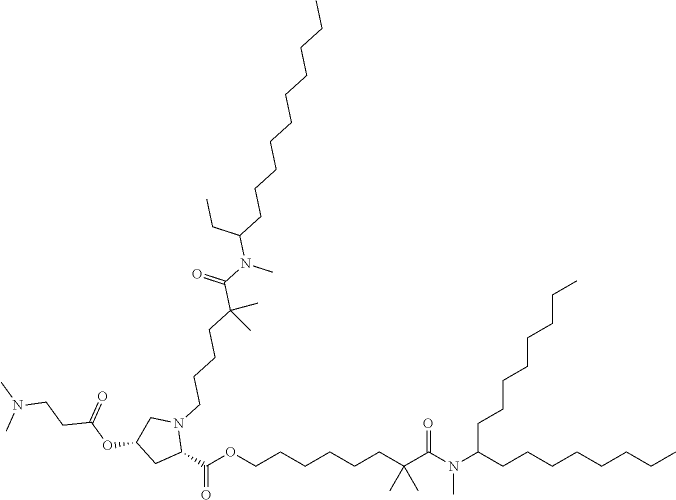 [SMILES: CCCCCCCCCCC(CC)N(C)C(=O)C(C)(C)CCCCN1C[C@@H](OC(=O)CCN(C)C)C[C@H]1C(=O)OCCCCCCC(C)(C)C(=O)N(C)C(CCCCCCCC)CCCCCCCC] | 8-(heptadecan-9- yl(methyl)amino)- 7,7-dimethyl-8- oxooctyl (2S,4S)-1- (5,5-dimethyl-6- (methyl(tridecan-3- yl)amino)-6- oxohexyl)-4-((3- (dimethylamino) propanoyl)oxy) pyrrolidine- 2-carboxylate |
| B8 | 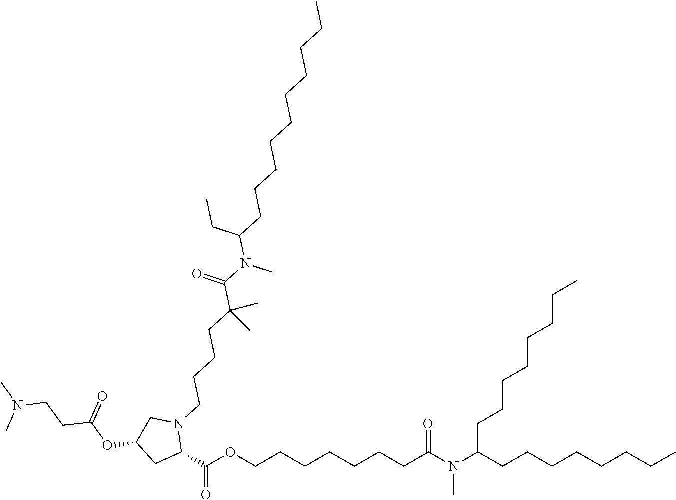 [SMILES: CCCCCCCCCCC(CC)N(C)C(=O)C(C)(C)CCCCN1C[C@@H](OC(=O)CCN(C)C)C[C@H]1C(=O)OCCCCCCCC(=O)N(C)C(CCCCCCCC)CCCCCCCC] | 8-(heptadecan-9- yl(methyl)amino)-8- oxooctyl (2S,4S)-1- (5,5-dimethyl-6- (methyl(tridecan-3- yl)amino)-6- oxohexyl)-4-((3- (dimethylamino) propanoyl)oxy) pyrrolidine- 2-carboxylate |
| B9 | 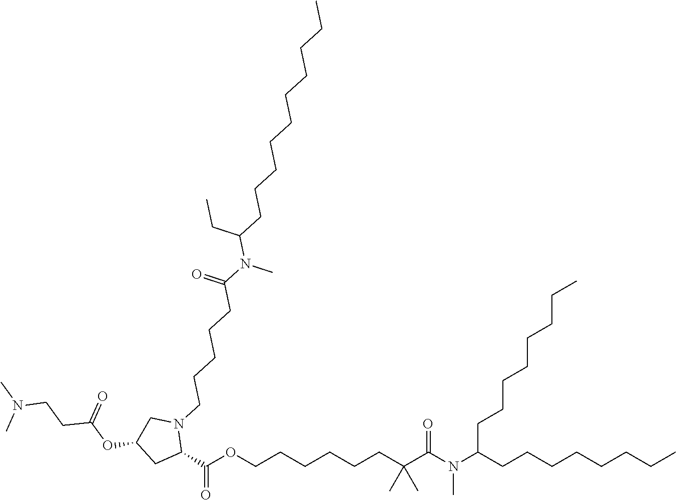 [SMILES: CCCCCCCCCCC(CC)N(C)C(=O)CCCCCN1C[C@@H](OC(=O)CCN(C)C)C[C@H]1C(=O)OCCCCCCC(C)(C)C(=O)N(C)C(CCCCCCCC)CCCCCCCC] | 8-(heptadecan-9- yl(methyl)amino)- 7,7-dimethyl-8- oxooctyl (2S,4S)-4- ((3- (dimethylamino) propanoyl)oxy)-1-(6- (methyl(tridecan-3- yl)amino)-6- oxohexyl)pyrrolidine- 2-carboxylate |
| B10 | 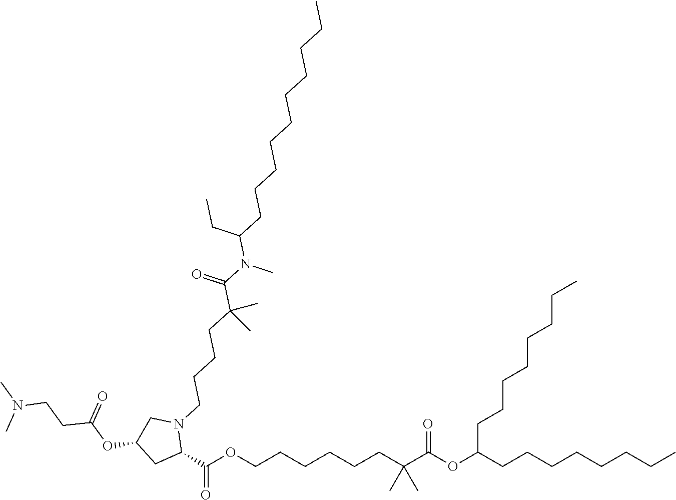 [SMILES: CCCCCCCCCCC(CC)N(C)C(=O)C(C)(C)CCCCN1C[C@@H](OC(=O)CCN(C)C)C[C@H]1C(=O)OCCCCCCC(C)(C)C(=O)OC(CCCCCCCC)CCCCCCCC] | 8-(heptadecan-9- yloxy)-7,7-dimethyl- 8-oxooctyl (2S,4S)- 1-(5,5-dimethyl-6- (methyl(tridecan-3- yl)amino)-6- oxohexyl)-4-((3- (dimethylamino) propanoyl)oxy) pyrrolidine- 2-carboxylate |
| B11 | 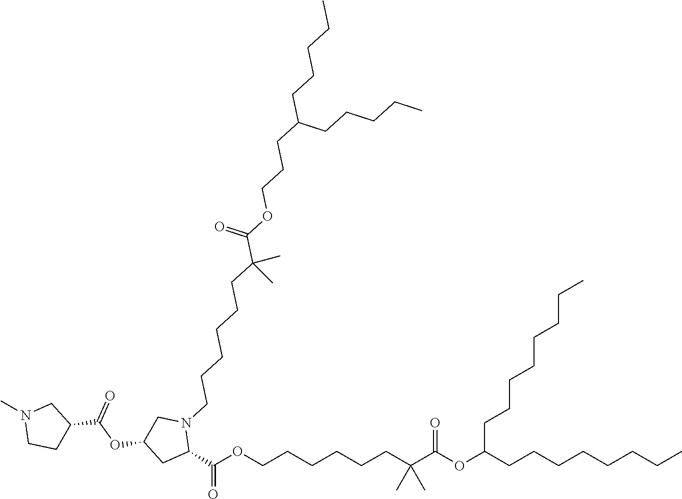 [SMILES: CCCCCCCCC(CCCCCCCC)OC(=O)C(C)(C)CCCCCCOC(=O)[C@@H]1C[C@H](OC(=O)[C@@H]2CCN(C)C2)CN1CCCCCCC(C)(C)C(=O)OCCCC(CCCCC)CCCCC] | 8-(heptadecan-9- yloxy)-7,7-dimethyl- 8-oxooctyl (2S,4S)- 1-(7,7-dimethyl-8- oxo-8-((4- pentylnonyl)oxy) octyl)-4-(((R)-1- methylpyrrolidine-3- carbonyl)oxy) pyrrolidine-2- carboxylate |
| B12 | 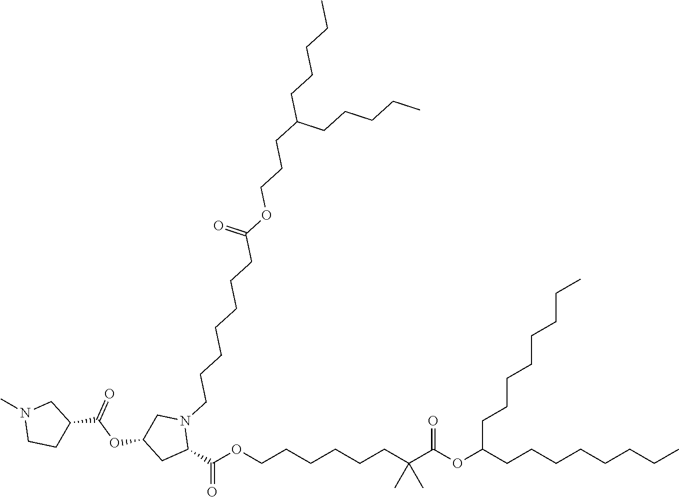 [SMILES: CCCCCCCCC(CCCCCCCC)OC(=O)C(C)(C)CCCCCCOC(=O)[C@@H]1C[C@H](OC(=O)[C@@H]2CCN(C)C2)CN1CCCCCCCC(=O)OCCCC(CCCCC)CCCCC] | 8-(heptadecan-9- yloxy)-7,7-dimethyl- 8-oxooctyl (2S,4S)- 4-(((R)-1- methylpyrrolidine-3- carbonyl)oxy)-1-(8- oxo-8-((4- pentylnonyl)oxy) octyl)pyrrolidine-2- carboxylate |
| B13 | 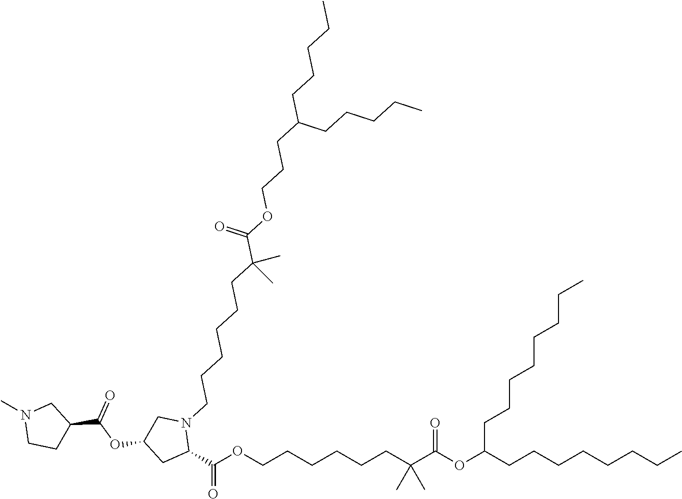 [SMILES: CCCCCCCCC(CCCCCCCC)OC(=O)C(C)(C)CCCCCCOC(=O)[C@@H]1C[C@H](OC(=O)[C@H]2CCN(C)C2)CN1CCCCCCC(C)(C)C(=O)OCCCC(CCCCC)CCCCC] | 8-(heptadecan-9- yloxy)-7,7-dimethyl- 8-oxooctyl (2S,4S)- 1-(7,7-dimethyl-8- oxo-8-((4- pentylnonyl)oxy) octyl)-4-(((S)-1- methylpyrrolidine-3- carbonyl)oxy) pyrrolidine-2- carboxylate |
| B14 | 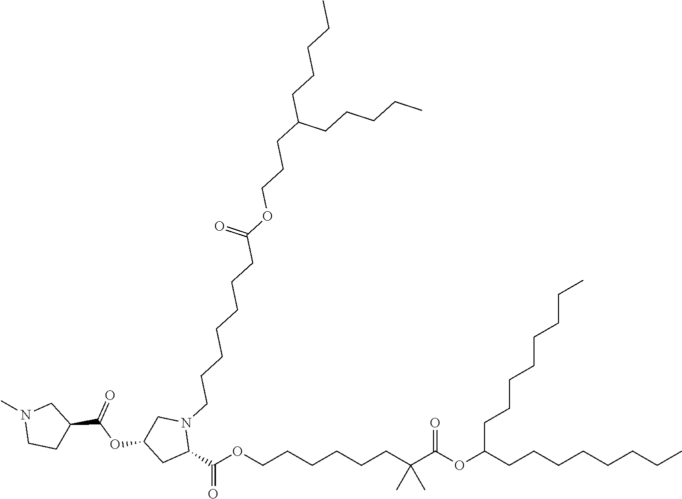 [SMILES: CCCCCCCCC(CCCCCCCC)OC(=O)C(C)(C)CCCCCCOC(=O)[C@@H]1C[C@H](OC(=O)[C@H]2CCN(C)C2)CN1CCCCCCCC(=O)OCCCC(CCCCC)CCCCC] | 8-(heptadecan-9- yloxy)-7,7-dimethyl- 8-oxooctyl (2S,4S)- 4-(((S)-1- methylpyrrolidine-3- carbonyl)oxy)-1-(8- oxo-8-((4- pentylnonyl)oxy) octyl)pyrrolidine-2- carboxylate |
| B15 | 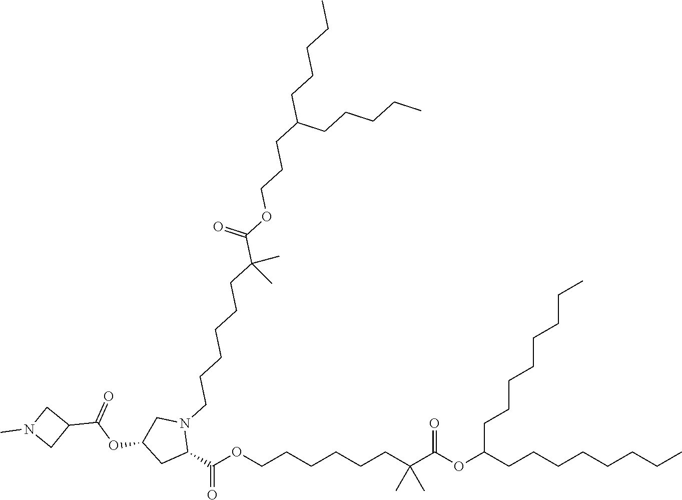 [SMILES: CCCCCCCCC(CCCCCCCC)OC(=O)C(C)(C)CCCCCCOC(=O)[C@@H]1C[C@H](OC(=O)C2CN(C)C2)CN1CCCCCCC(C)(C)C(=O)OCCCC(CCCCC)CCCCC] | 8-(heptadecan-9- yloxy)-7,7-dimethyl- 8-oxooctyl (2S,4S)- 1-(7,7-dimethyl-8- oxo-8-((4- pentylnonyl)oxy) octyl)-4-((1- methylazetidine-3- carbonyl)oxy) pyrrolidine-2- carboxylate |
| B16 | 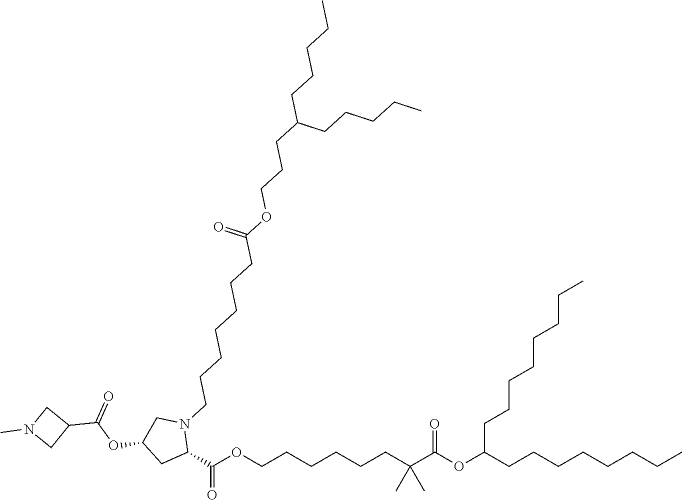 [SMILES: CCCCCCCCC(CCCCCCCC)OC(=O)C(C)(C)CCCCCCOC(=O)[C@@H]1C[C@H](OC(=O)C2CN(C)C2)CN1CCCCCCCC(=O)OCCCC(CCCCC)CCCCC] | 8-(heptadecan-9- yloxy)-7,7-dimethyl- 8-oxooctyl (2S,4S)- 4-((1- methylazetidine-3- carbonyl)oxy)-1-(8- oxo-8-((4- pentylnonyl)oxy) octyl)pyrrolidine-2- carboxylate |
| B17 | 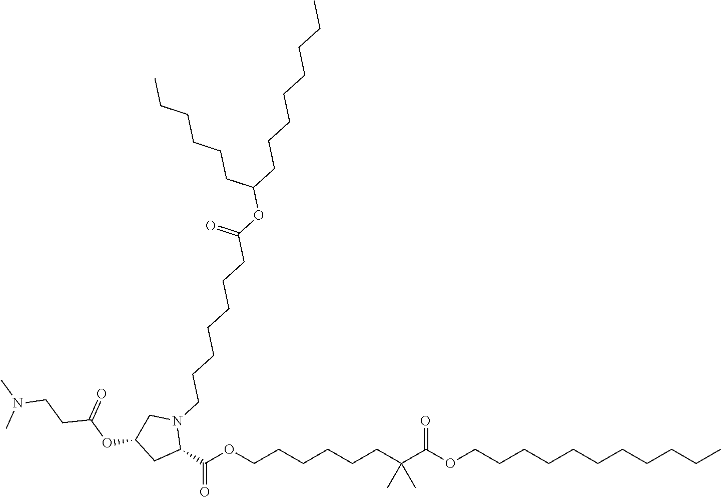 [SMILES: CCCCCCCCCCCOC(=O)C(C)(C)CCCCCCOC(=O)[C@@H]1C[C@H](OC(=O)CCN(C)C)CN1CCCCCCCC(=O)OC(CCCCCC)CCCCCCCC] | 7,7-dimethyl-8-oxo- 8-(undecyloxy)octyl (2S,4S)-4-((3- (dimethylamino) propanoyl)oxy)- 1-(8-oxo- 8-(pentadecan-7- yloxy)octyl) pyrrolidine- 2-carboxylate |
| B18 | 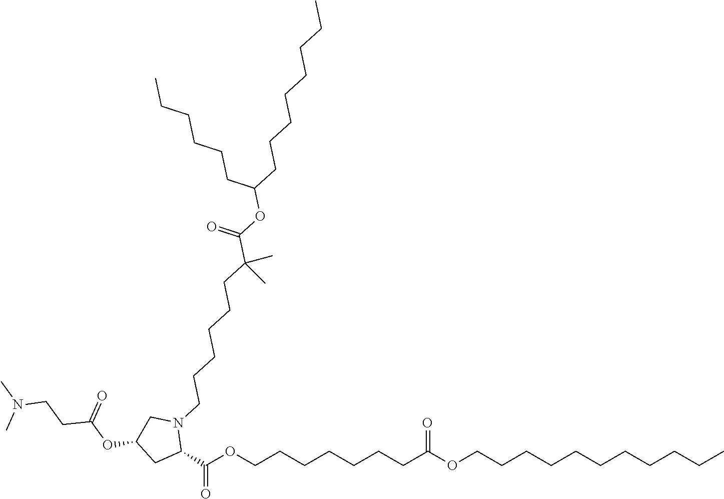 [SMILES: CCCCCCCCCCCOC(=O)CCCCCCCOC(=O)[C@@H]1C[C@H](OC(=O)CCN(C)C)CN1CCCCCCC(C)(C)C(=O)OC(CCCCCC)CCCCCCCC] | 8-oxo-8- (undecyloxy)octyl (2S,4S)-1-(7,7- dimethyl-8-oxo-8- (pentadecan-7- yloxy)octyl)-4-((3- (dimethylamino) propanoyl)oxy) pyrrolidine- 2-carboxylate |
| B19 | 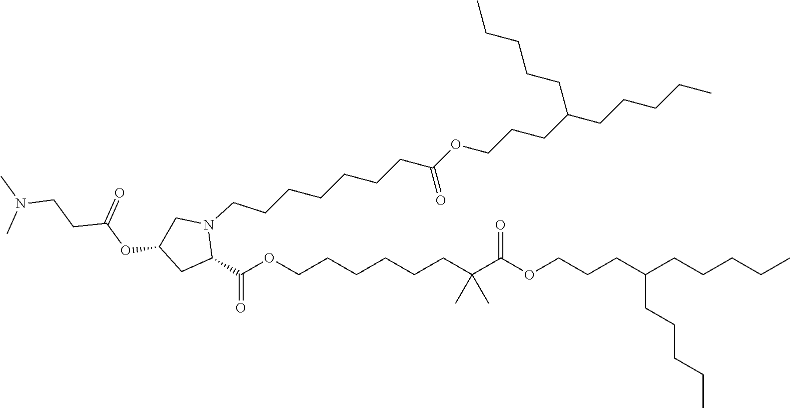 [SMILES: CCCCCC(CCCCC)CCCOC(=O)CCCCCCCN1C[C@@H](OC(=O)CCN(C)C)C[C@H]1C(=O)OCCCCCCC(C)(C)C(=O)OCCCC(CCCCC)CCCCC] | 7,7-dimethyl-8-oxo- 8-((4- pentylnonyl)oxy)octyl (2S,4S)-4-((3- (dimethylamino) propanoyl)oxy)- 1-(8-oxo- 8-((4- pentylnonyl)oxy)octyl) pyrrolidine-2- carboxylate |
| B20 | 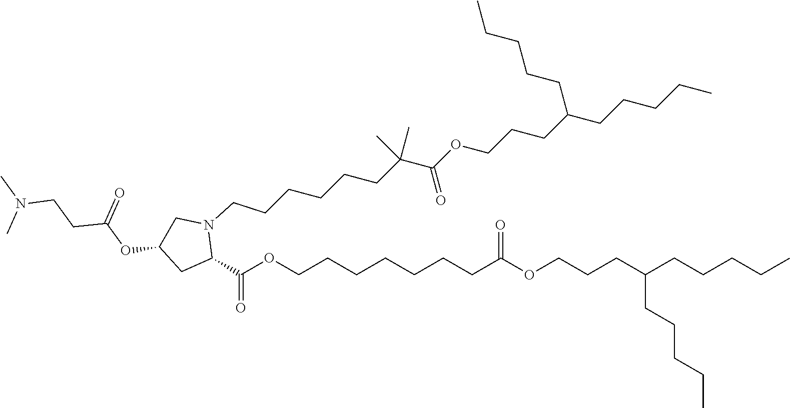 [SMILES: CCCCCC(CCCCC)CCCOC(=O)CCCCCCCOC(=O)[C@@H]1C[C@H](OC(=O)CCN(C)C)CN1CCCCCCC(C)(C)C(=O)OCCCC(CCCCC)CCCCC] | 8-oxo-8-((4- pentylnonyl)oxy)octyl (2S,4S)-1-(7,7- dimethyl-8-oxo-8- ((4- pentylnonyl)oxy) octyl)-4-((3- (dimethylamino) propanoyl)oxy) pyrrolidine- 2-carboxylate |
| B21 | 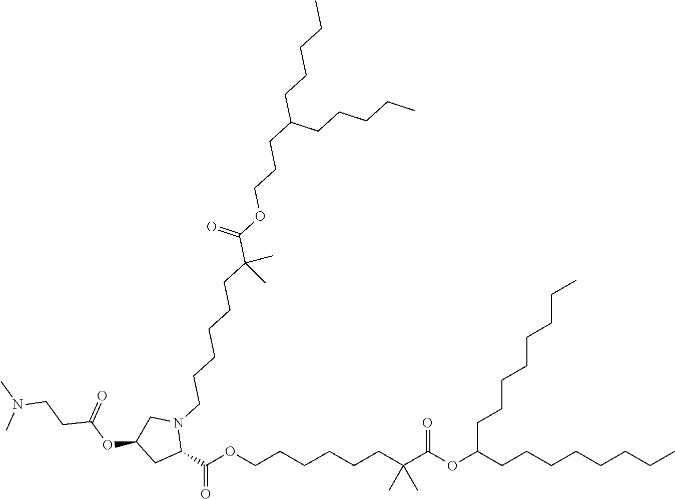 [SMILES: CCCCCCCCC(CCCCCCCC)OC(=O)C(C)(C)CCCCCCOC(=O)[C@@H]1C[C@@H](OC(=O)CCN(C)C)CN1CCCCCCC(C)(C)C(=O)OCCCC(CCCCC)CCCCC] | 8-(heptadecan-9- yloxy)-7,7-dimethyl- 8-oxooctyl (2S,4R)- 1-(7,7-dimethyl-8- oxo-8-((4- pentylnonyl)oxy) octyl)-4-((3- (dimethylamino) propanoyl)oxy) pyrrolidine- 2-carboxylate |
| B22 | 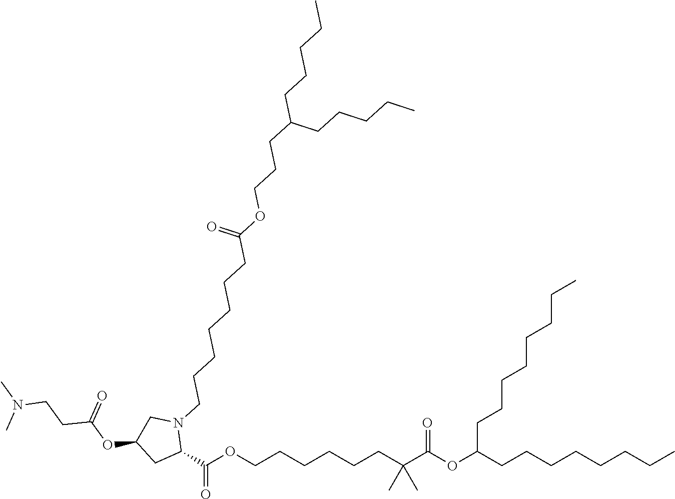 [SMILES: CCCCCCCCC(CCCCCCCC)OC(=O)C(C)(C)CCCCCCOC(=O)[C@@H]1C[C@@H](OC(=O)CCN(C)C)CN1CCCCCCCC(=O)OCCCC(CCCCC)CCCCC] | 8-(heptadecan-9- yloxy)-7,7-dimethyl- 8-oxooctyl (2S,4R)- 4-((3- (dimethylamino) propanoyl)oxy)- 1-(8-oxo- 8-((4- pentylnonyl)oxy)octyl) pyrrolidine-2- carboxylate |
| B23 | 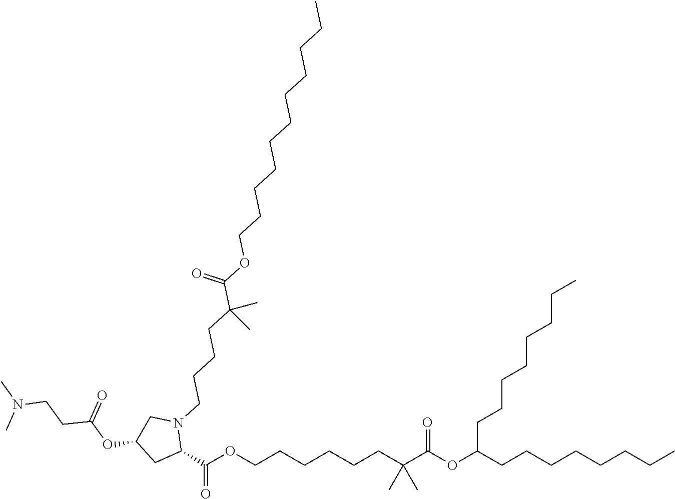 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCCCC)CCCCCCCC] | 8-(heptadecan-9- yloxy)-7,7-dimethyl- 8-oxooctyl (2S,4S)- 1-(5,5-dimethyl-6- oxo-6- (undecyloxy)hexyl)- 4-((3- (dimethylamino) propanoyl)oxy) pyrrolidine- 2-carboxylate |
| B24 | 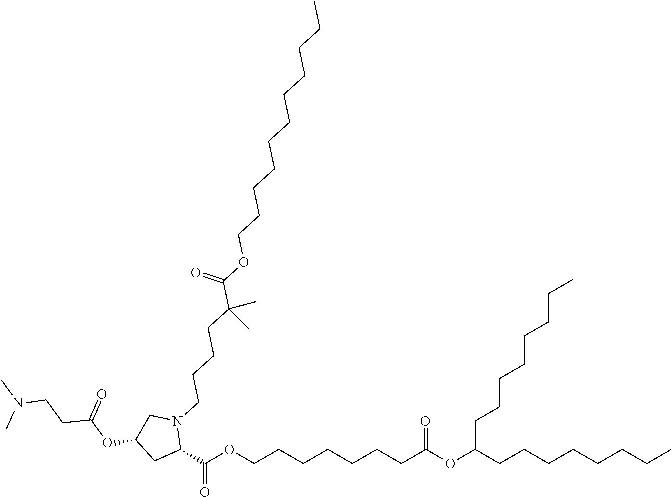 [SMILES: CCCCCCCCCCCOC(=O)C(C)(C)CCCCN1C[C@@H](OC(=O)CCN(C)C)C[C@H]1C(=O)OCCCCCCCC(=O)OC(CCCCCCCC)CCCCCCCC] | 8-(heptadecan-9- yloxy)-8-oxooctyl (2S,4S)-1-(5,5- dimethyl-6-oxo-6- (undecyloxy)hexyl)- 4-((3- (dimethylamino) propanoyl)oxy) pyrrolidine- 2-carboxylate |
| B25 | 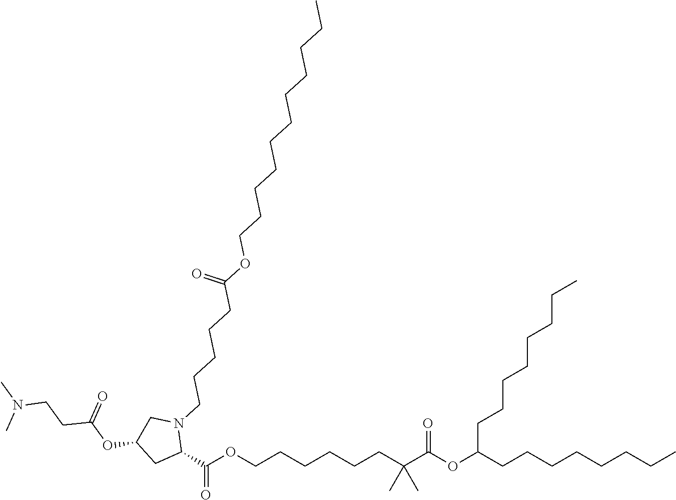 [SMILES: CCCCCCCCCCCOC(=O)CCCCCN1C[C@@H](OC(=O)CCN(C)C)C[C@H]1C(=O)OCCCCCCC(C)(C)C(=O)OC(CCCCCCCC)CCCCCCCC] | 8-(heptadecan-9- yloxy)-7,7-dimethyl- 8-oxooctyl (2S,4S)- 4-((3- (dimethylamino) propanoyl)oxy)- 1-(6-oxo-6- (undecyloxy)hexyl) pyrrolidine-2- carboxylate |

[symbol]

indicates data missing or illegible when filed

In some embodiments, a lipid membrane of the LNMPs comprises at least 35% of the lipid compound from group i), e.g., at least 40%, 45%, 50%, 55%, 60%, 65%, 70%, 75% 80%, 85%, 90%, or more than 90% of the lipid compound from group i), e.g., 35%-40%, 40%-50%, 50%-60%, 60%-70%, 70%-80%, or 80%-90% of the lipid compound from group i).

In some embodiments, a lipid membrane of the LNMPs comprises at least 35% of the lipid compound from group ii), e.g., at least 40%, 45%, 50%, 55%, 60%, 65%, 70%, 75% 80%, 85%, 90%, or more than 90% of the lipid compound from group ii), e.g., 35%-40%, 40%-50%, 50%-60%, 60%-70%, 70%-80%, or 80%-90% of the lipid compound from group ii).

In some embodiments, a lipid membrane of the LNMPs comprises at least 35% of the lipid compound from group iii), e.g., at least 40%, 45%, 50%, 55%, 60%, 65%, 70%, 75% 80%, 85%, 90%, or more than 90% of the lipid compound from group iii), e.g., 35%-40%, 40%-50%, 50%-60%, 60%-70%, 70%-80%, or 80%-90% of the lipid compound from group iii).

In some embodiments, a lipid membrane of the LNMPs comprises at least 35% of the lipid compound from group iii), e.g., at least 40%, 45%, 50%, 55%, 60%, 65%, 70%, 75% 80%, 85%, 90%, or more than 90% of the lipid compound from group iii), e.g., 35%-40%, 40%-50%, 50%-60%, 60%-70%, 70%-80%, or 80%-90% of the lipid compound from group iv).

In some instances, the LNMPs comprise at least 1%, 5%, 10%, 20%, 30%, 40%, 50%, 60%, 70%, 80%, 90%, or more than 90% ionizable lipid.

In some instances, the LNMPs comprise a molar ratio of at least 0.1%, 1%, 5%, 10%, 15%, 20%, 25%, 30%, 35%, 40%, 45%, 50%, 55%, 60%, 65%, 70%, 75% 80%, 85%, 90%, or more than 90% ionizable lipid, e.g., 1%-10%, 10%-20%, 20%-30%, 30%-40%, 40%-50%, 50%-60%, 60%-70%, 70%-80%, or 80%-90% ionizable lipid, e.g., about 25%-75% ionizable lipid (e.g., about 25%-75% ionizable lipid).

### Other Ionizable Lipids

In the LNMP formulations, more than one ionizable lipid can be used for the ionizable lipid component: one or more of the ionizable lipids from the compounds of formulas in groups i)-iv) can be used alone or in combination with a different ionizable lipid from the compounds of formulas in groups i)-iv).

In some embodiments, the ionizable lipid do not include 1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5 (Moderna), and 98N12-5.

In some embodiments, the additional ionizable lipid is selected from the group consisting of 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315.

In some embodiments, the additional ionizable lipid is represented by the following formula III:

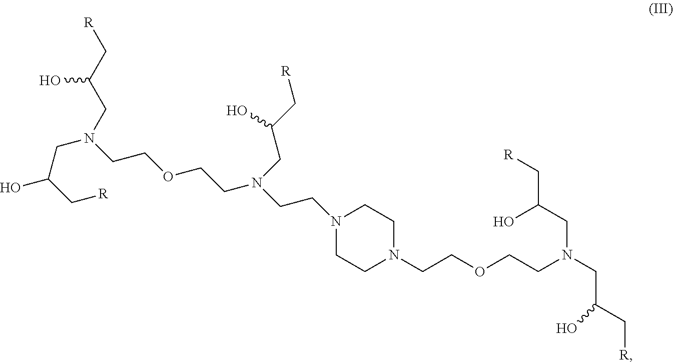

wherein R is C₈-C₁₄ alkyl group.

The ionizable lipid described herein may include an amine core described herein substituted with one or more (e.g., 1, 2, 3, 4, 5, or 6) lipid tails. In some embodiments, the ionizable lipids described herein include at least 3 lipid tails. A lipid tail may be a C₈-C₁₈ hydrocarbon (e.g., C₆-C₁₈ alkyl or C₆-C₁₈ alkanoyl). An amine core may be substituted with one or more lipid tails at a nitrogen atom (e.g., one hydrogen atom attached to the nitrogen atom may be replaced with a lipid tail).

In some embodiments, the amine core has a structure of:

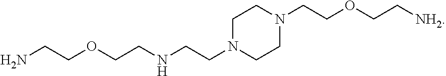 [SMILES: NCCOCCNCCN1CCN(CCOCCN)CC1]

In some embodiments, the amine core has a structure of:

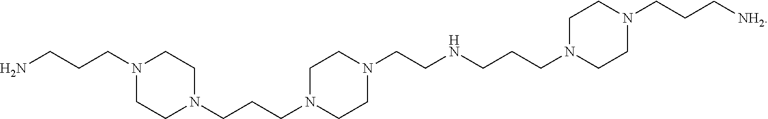 [SMILES: NCCCN1CCN(CCCNCCN2CCN(CCCN3CCN(CCCN)CC3)CC2)CC1]

In some embodiments, the amine core has a structure of:

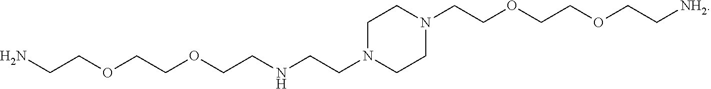 [SMILES: NCCOCCOCCNCCN1CCN(CCOCCOCCN)CC1]

In some embodiments, the amine core has a structure of:

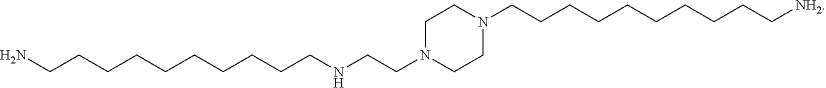 [SMILES: NCCCCCCCCCCNCCN1CCN(CCCCCCCCCCN)CC1]

In some embodiments, the amine core has a structure of:

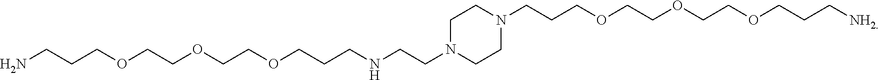 [SMILES: NCCCOCCOCCOCCCNCCN1CCN(CCCOCCOCCOCCCN)CC1]

In some embodiments, the amine core has a structure of:

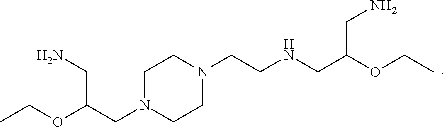 [SMILES: CCOC(CN)CNCCN1CCN(CC(CN)OCC)CC1]

In some embodiments, the amine core has a structure of:

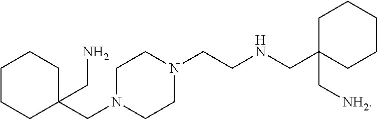 [SMILES: NCC1(CNCCN2CCN(CC3(CN)CCCCC3)CC2)CCCCC1]

In some embodiments, the amine core has a structure of:

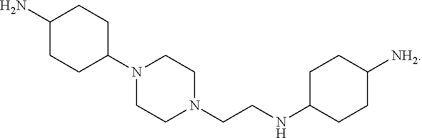 [SMILES: NC1CCC(NCCN2CCN(C3CCC(N)CC3)CC2)CC1]

Other suitable lipids for use in the RNA composition (e.g., the nucleic acid vaccine composition) and methods for making and using thereof include the lipids as described in International Patent Publication WO 2016/118725, which is incorporated herein by reference in its entirety.

In certain embodiments, the RNA composition (e.g., the nucleic acid vaccine composition) and methods for making and using thereof include a lipid having a compound structure of:

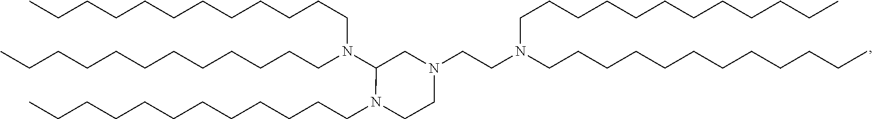 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable lipids for use in the RNA composition (e.g., the nucleic acid vaccine composition) and methods for making and using thereof include the lipids as described in International Patent Publication WO 2016/118724, which is incorporated herein by reference in its entirety.

In certain embodiments, the RNA composition (e.g., the nucleic acid vaccine composition) and methods for making and using thereof include a lipid having a compound structure of:

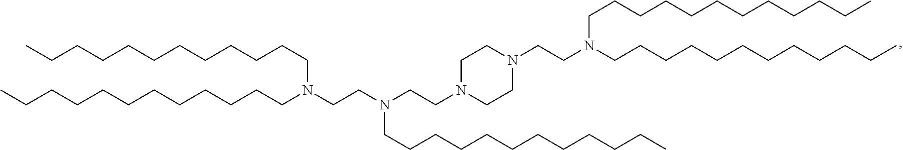 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable lipids for use in the RNA composition (e.g., the nucleic acid vaccine composition) and methods for making and using thereof include a lipid having the formula of 14,25-ditridecyl 15,18,21,24-tetraaza-octatriacontane, and pharmaceutically acceptable salts thereof.

Other suitable lipids for use in the RNA composition (e.g., the nucleic acid vaccine composition) and methods for making and using thereof include the lipids as described in International Patent Publications WO 2013/063468 and WO 2016/205691, each of which is incorporated herein by reference in its entirety.

In some embodiments, the RNA composition (e.g., the nucleic acid vaccine composition) and methods for making and using thereof include a lipid of the following formula:

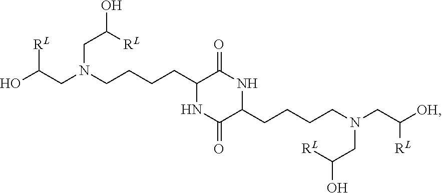

or pharmaceutically acceptable salts thereof, wherein each instance of R^{L} is independently optionally substituted C6-C40 alkenyl.

In certain embodiments, the RNA composition (e.g., the nucleic acid vaccine composition) and methods for making and using thereof include a lipid having a compound structure of:

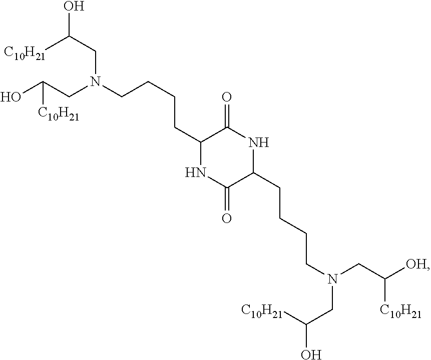

and pharmaceutically acceptable salts thereof.

In certain embodiments, the RNA composition (e.g., the nucleic acid vaccine composition) and methods for making and using thereof include a lipid having a compound structure of:

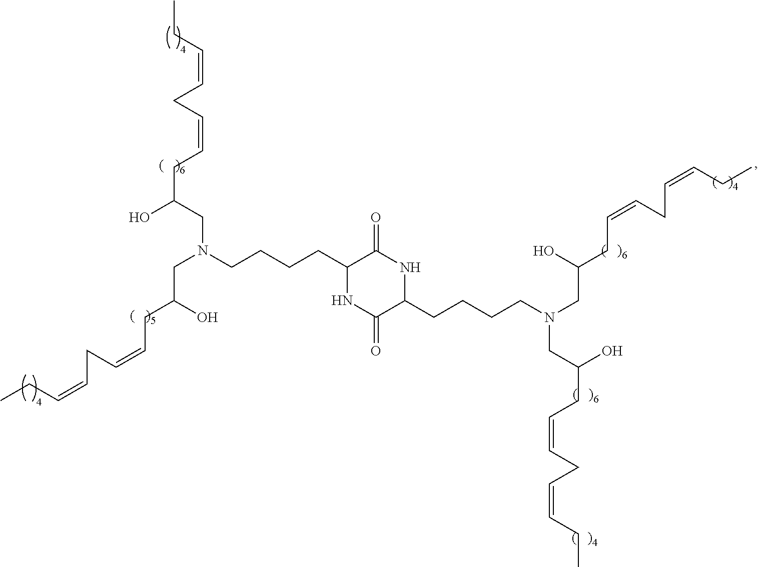

and pharmaceutically acceptable salts thereof.

In certain embodiments, the RNA composition (e.g., the nucleic acid vaccine composition) and methods for making and using thereof include a lipid having a compound structure of:

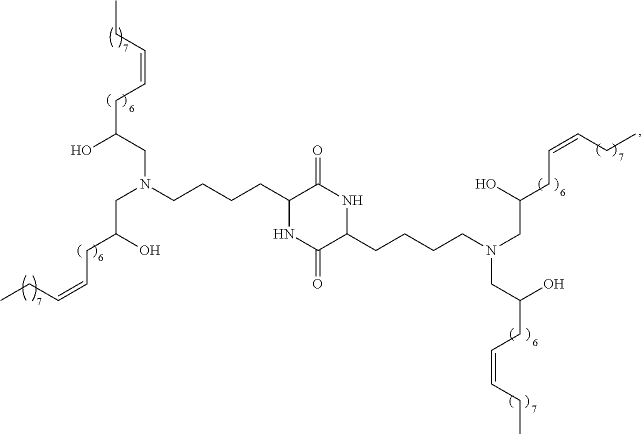

and pharmaceutically acceptable salts thereof.

In certain embodiments, the RNA composition (e.g., the nucleic acid vaccine composition) and methods for making and using thereof include a lipid having a compound structure of:

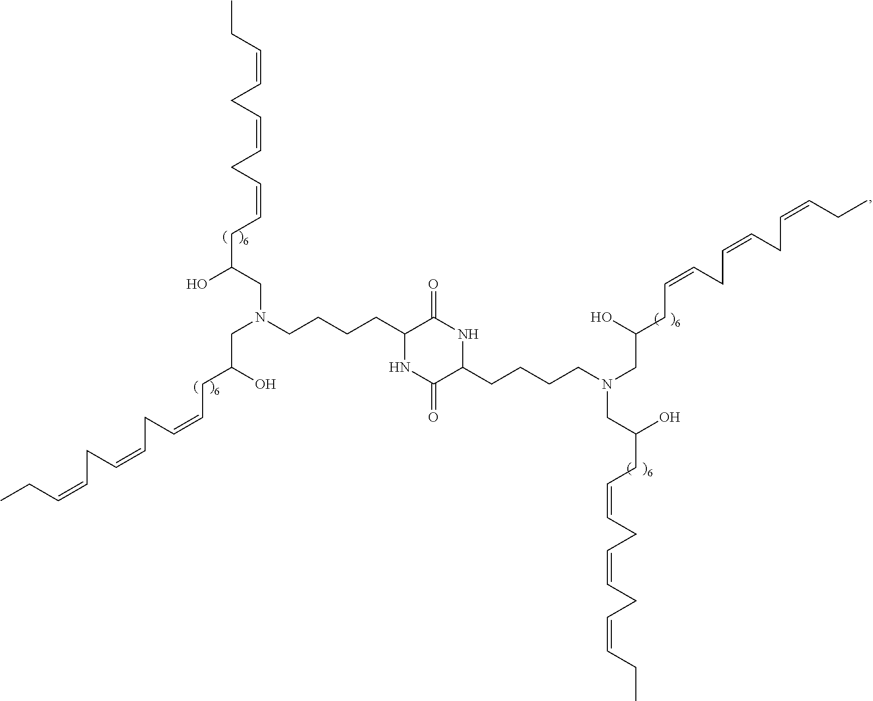

and pharmaceutically acceptable salts thereof.

Other suitable lipids for use in the RNA composition (e.g., the nucleic acid vaccine composition) and methods for making and using thereof include the lipids as described in International Patent Publication WO 2015/184256, which is incorporated herein by reference in its entirety. In some embodiments, the RNA composition (e.g., the nucleic acid vaccine composition) and methods for making and using thereof include a lipid of the following formula:

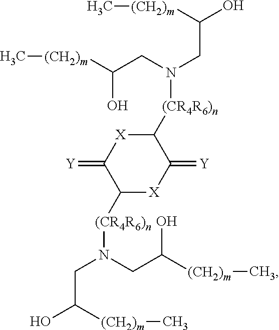

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each RB is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen.

In certain embodiments, the RNA composition (e.g., the nucleic acid vaccine composition) and methods for making and using thereof include a lipid, “Target 23”, having a compound structure of:

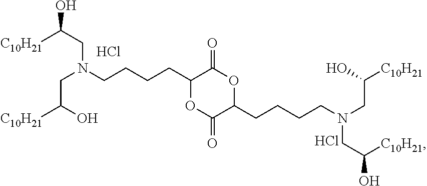

(Target 23) and pharmaceutically acceptable salts thereof.

Other suitable lipids for use in the RNA composition (e.g., the nucleic acid vaccine composition) and methods for making and using thereof include the lipids as described in International Patent Publication WO 2016/004202, which is incorporated herein by reference in its entirety.

In some embodiments, the RNA composition (e.g., the nucleic acid vaccine composition) and methods for making and using thereof include a lipid having the compound structure:

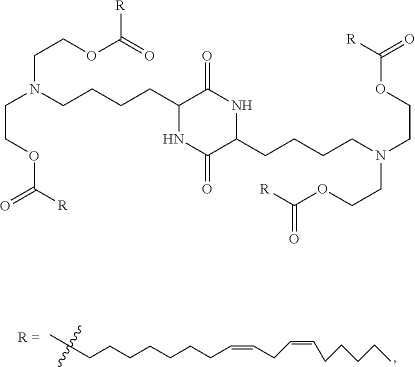

or a pharmaceutically acceptable salt thereof.

In some embodiments, the RNA composition (e.g., the nucleic acid vaccine composition) and methods for making and using thereof include a lipid having the compound structure:

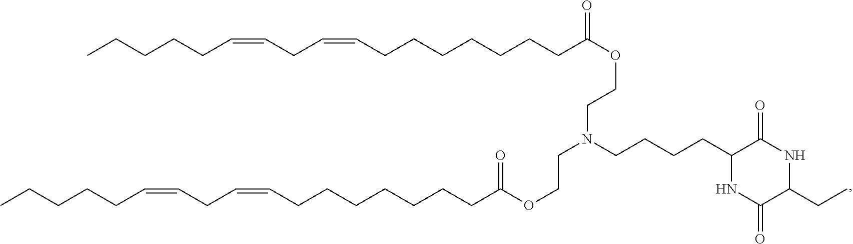 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof.

In some embodiments, the RNA composition (e.g., the nucleic acid vaccine composition) and methods for making and using thereof include a lipid having the compound structure:

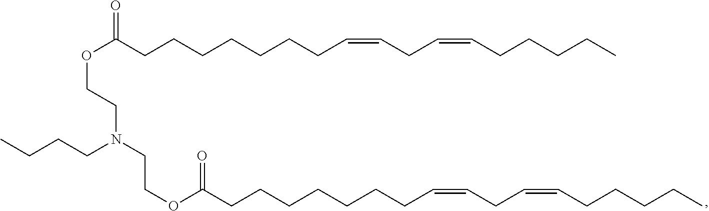 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable lipids for use in the RNA composition (e.g., the nucleic acid vaccine composition) and methods for making and using thereof include lipids as described in U.S. Provisional Patent Application Ser. No. 62/758,179, which is incorporated herein by reference in its entirety.

In some embodiments, the RNA composition (e.g., the nucleic acid vaccine composition) and methods for making and using thereof include a lipid of the following formula:

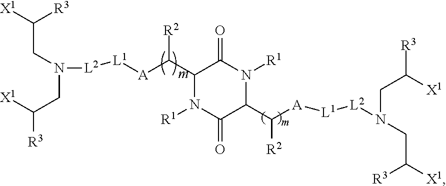

or a pharmaceutically acceptable salt thereof, wherein each R¹ and R² is independently H or C1-C6 aliphatic; each m is independently an integer having a value of 1 to 4; each A is independently a covalent bond or arylene; each L¹ is independently an ester, thioester, disulfide, or anhydride group; each L² is independently C₂-C₁₀ aliphatic; each X¹ is independently H or OH; and each R³ is independently C6-C20 aliphatic.

In some embodiments, the RNA composition (e.g., the nucleic acid vaccine composition) and methods for making and using thereof include a lipid of the following formula:

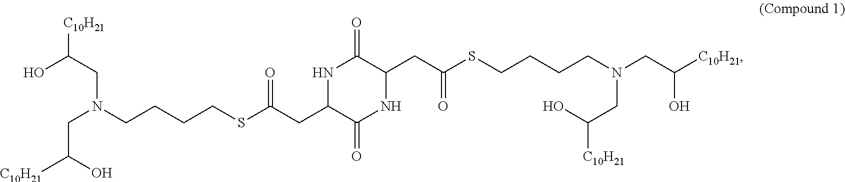

or a pharmaceutically acceptable salt thereof.

In some embodiments, the RNA composition (e.g., the nucleic acid vaccine composition) and methods for making and using thereof include a lipid of the following formula:

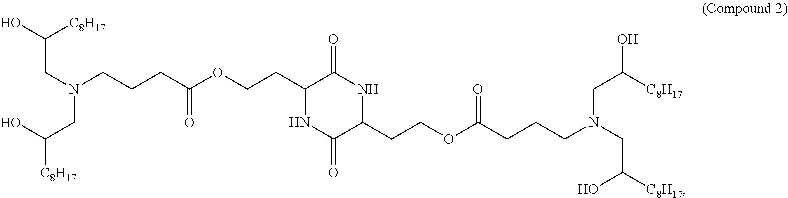

or a pharmaceutically acceptable salt thereof.

In some embodiments, the RNA composition (e.g., the nucleic acid vaccine composition) and methods for making and using thereof include a lipid of the following formula:

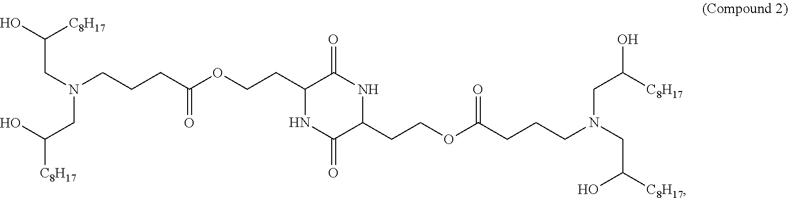

or a pharmaceutically acceptable salt thereof.

Other suitable lipids for use in the RNA composition (e.g., the nucleic acid vaccine composition) and methods for making and using thereof include the lipids as described in J. McClellan, M. C. King, Cell 2010, 141, 210-217 and in Whitehead et al., Nature Communications (2014) 5:4277, which is incorporated herein by reference in its entirety.

In certain embodiments, the lipids of the RNA composition (e.g., the nucleic acid vaccine composition) and methods for making and using thereof include a lipid having a compound structure of:

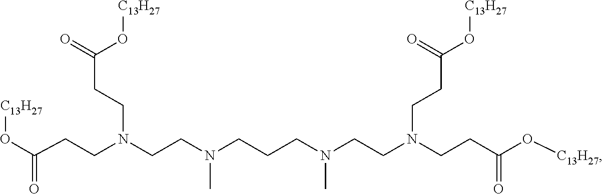

and pharmaceutically acceptable salts thereof.

Other suitable lipids for use in the RNA composition (e.g., the nucleic acid vaccine composition) and methods for making and using thereof include the lipids as described in International Patent Publication WO 2015/199952, which is incorporated herein by reference in its entirety.

In some embodiments, the RNA composition (e.g., the nucleic acid vaccine composition) and methods for making and using thereof include a lipid having the compound structure:

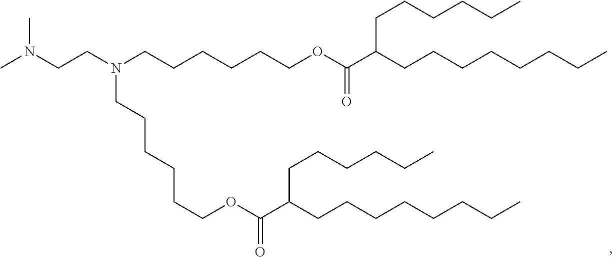 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof.

In some embodiments, the RNA composition (e.g., the nucleic acid vaccine composition) and methods for making and using thereof include a lipid having the compound structure:

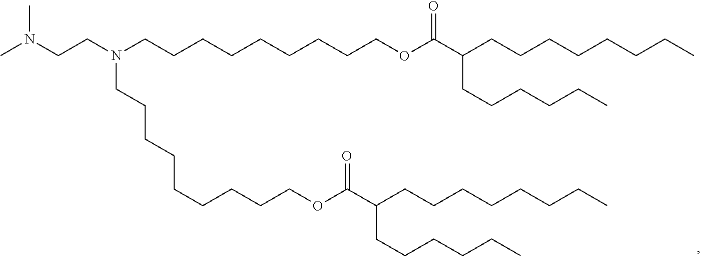 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof.

In some embodiments, the RNA composition (e.g., the nucleic acid vaccine composition) and methods for making and using thereof include a lipid having the compound structure:

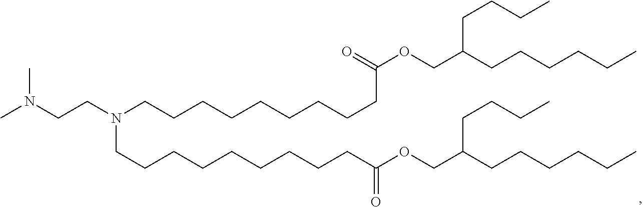 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof.

In some embodiments, the RNA composition (e.g., the nucleic acid vaccine composition) and methods for making and using thereof include a lipid having the compound structure:

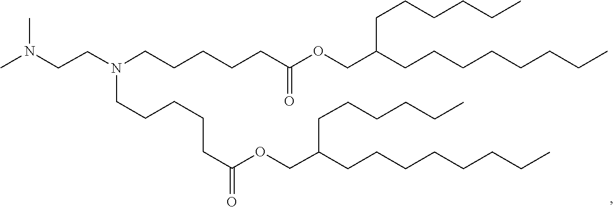 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof.

In some embodiments, the RNA composition (e.g., the nucleic acid vaccine composition) and methods for making and using thereof include a lipid having the compound structure:

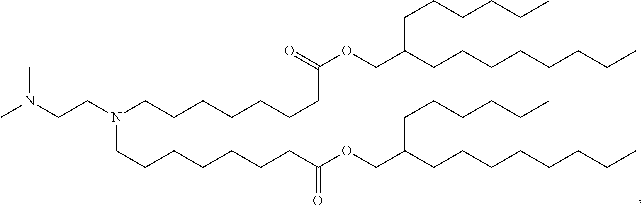 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof.

In some embodiments, the RNA composition (e.g., the nucleic acid vaccine composition) and methods for making and using thereof include a lipid having the compound structure:

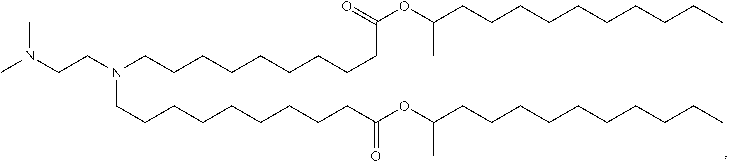 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof.

In some embodiments, the RNA composition (e.g., the nucleic acid vaccine composition) and methods for making and using thereof include a lipid having the compound structure:

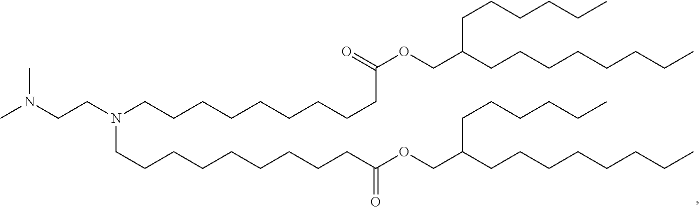 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof.

In some embodiments, the RNA composition (e.g., the nucleic acid vaccine composition) and methods for making and using thereof include a lipid having the compound structure:

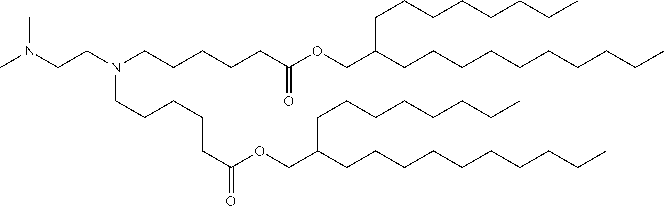 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof.

In some embodiments, the RNA composition (e.g., the nucleic acid vaccine composition) and methods for making and using thereof include a lipid having the compound structure:

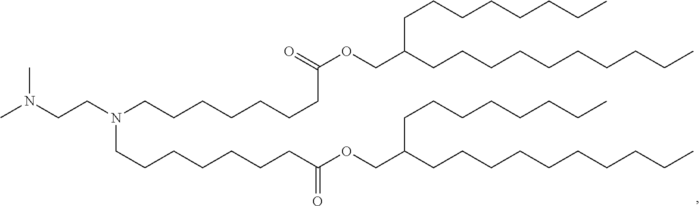 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof.

In some embodiments, the RNA composition (e.g., the nucleic acid vaccine composition) and methods for making and using thereof include a lipid having the compound structure:

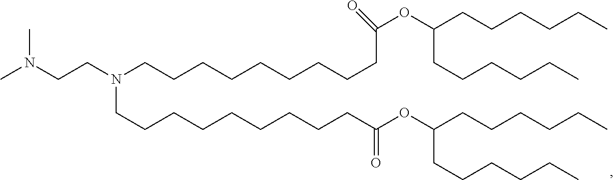 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof.

In some embodiments, the RNA composition (e.g., the nucleic acid vaccine composition) and methods for making and using thereof include a lipid having the compound structure:

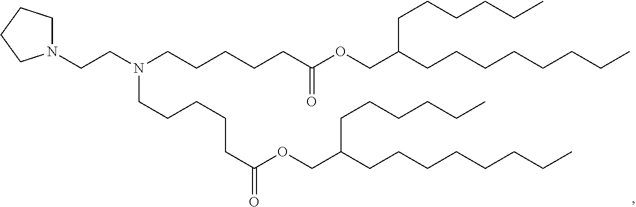 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof.

In some embodiments, the RNA composition (e.g., the nucleic acid vaccine composition) and methods for making and using thereof include a lipid having the compound structure:

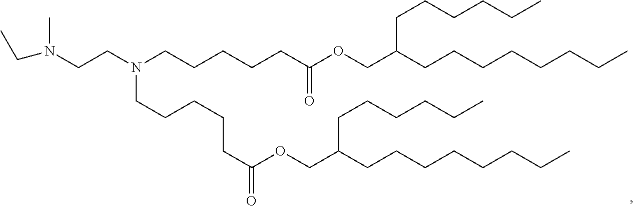 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof.

In some embodiments, the RNA composition (e.g., the nucleic acid vaccine composition) and methods for making and using thereof include a lipid having the compound structure:

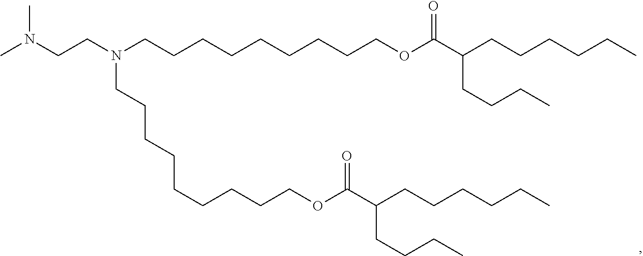 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable lipids for use in the RNA composition (e.g., the nucleic acid vaccine composition) and methods for making and using thereof include the lipids as described in International Patent Publication WO 2017/004143, which is incorporated herein by reference in its entirety.

In some embodiments, the RNA composition (e.g., the nucleic acid vaccine composition) and methods for making and using thereof include a lipid having the compound structure:

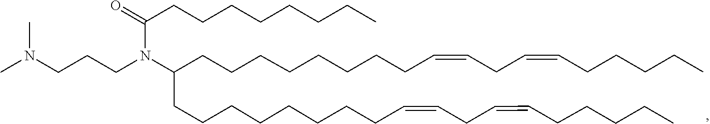 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof.

In some embodiments, the RNA composition (e.g., the nucleic acid vaccine composition) and methods for making and using thereof include a lipid having the compound structure:

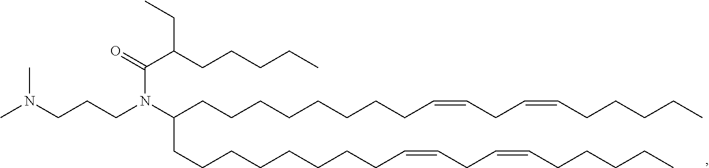 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof.

In some embodiments, the RNA composition (e.g., the nucleic acid vaccine composition) and methods for making and using thereof include a lipid having the compound structure:

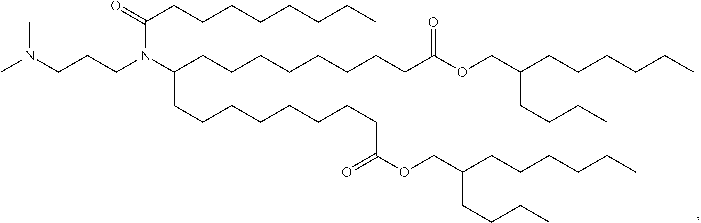 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof.

In some embodiments, the RNA composition (e.g., the nucleic acid vaccine composition) and methods for making and using thereof include a lipid having the compound structure:

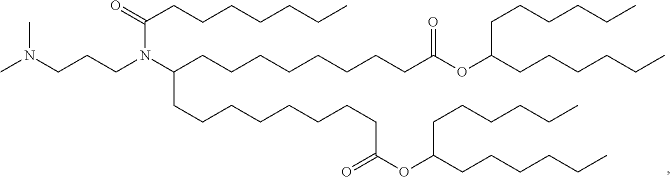 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof.

In some embodiments, the RNA composition (e.g., the nucleic acid vaccine composition) and methods for making and using thereof include a lipid having the compound structure:

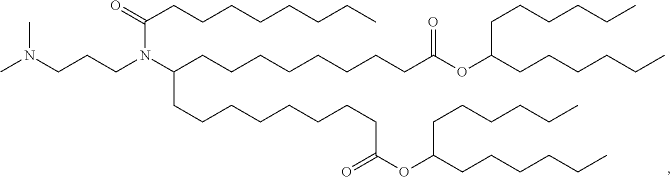 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof.

In some embodiments, the RNA composition (e.g., the nucleic acid vaccine composition) and methods for making and using thereof include a lipid having the compound structure:

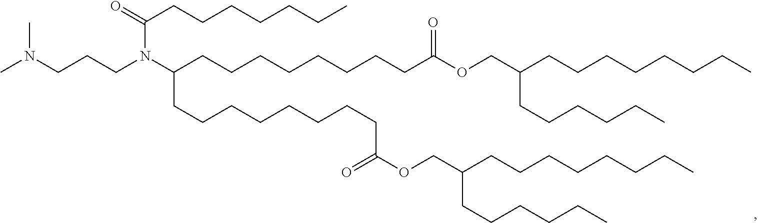 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof.

In some embodiments, the RNA composition (e.g., the nucleic acid vaccine composition) and methods for making and using thereof include a lipid having the compound structure:

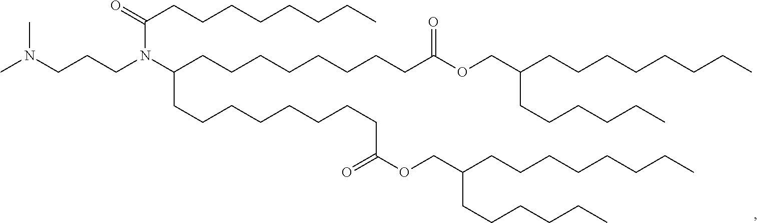 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof.

In some embodiments, the RNA composition (e.g., the nucleic acid vaccine composition) and methods for making and using thereof include a lipid having the compound structure:

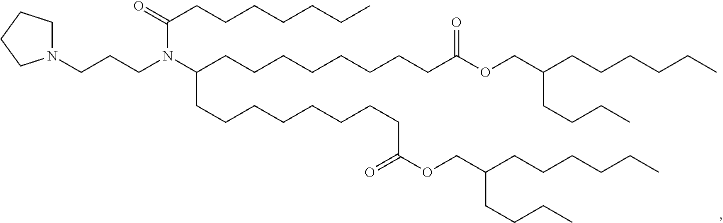 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof.

In some embodiments, the RNA composition (e.g., the nucleic acid vaccine composition) and methods for making and using thereof include a lipid having the compound structure:

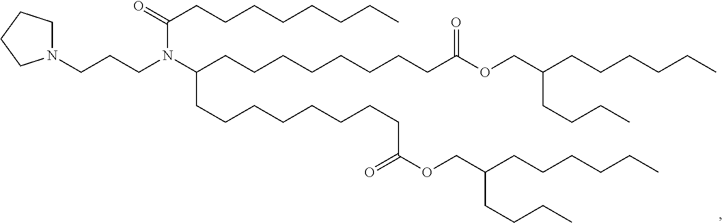 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof.

In some embodiments, the RNA composition (e.g., the nucleic acid vaccine composition) and methods for making and using thereof include a lipid having the compound structure:

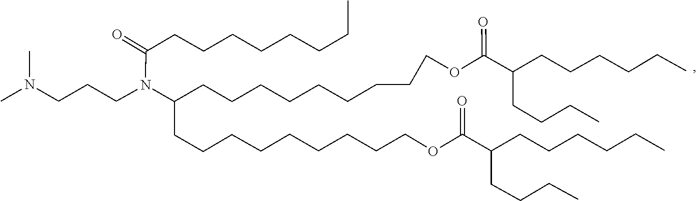 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof.

In some embodiments, the RNA composition (e.g., the nucleic acid vaccine composition) and methods for making and using thereof include a lipid having the compound structure:

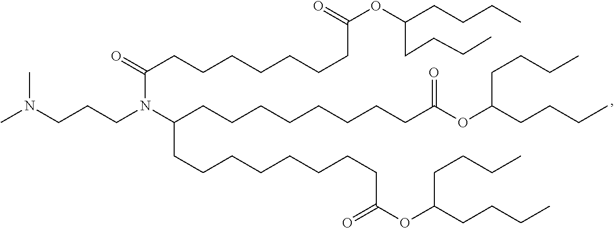 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof.

In some embodiments, the RNA composition (e.g., the nucleic acid vaccine composition) and methods for making and using thereof include a lipid having the compound structure:

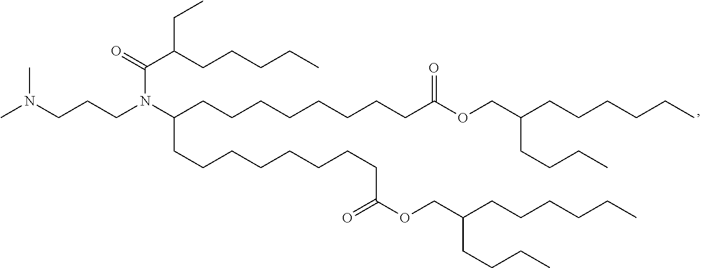 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof.

In some embodiments, the RNA composition (e.g., the nucleic acid vaccine composition) and methods for making and using thereof include a lipid having the compound structure:

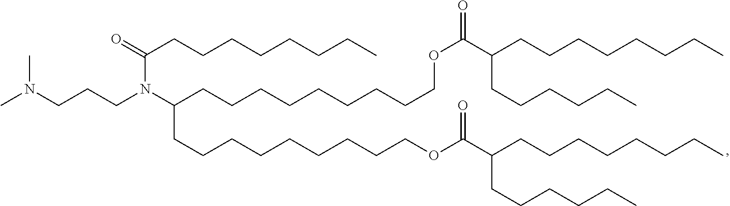 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof.

In some embodiments, the RNA composition (e.g., the nucleic acid vaccine composition) and methods for making and using thereof include a lipid having the compound structure:

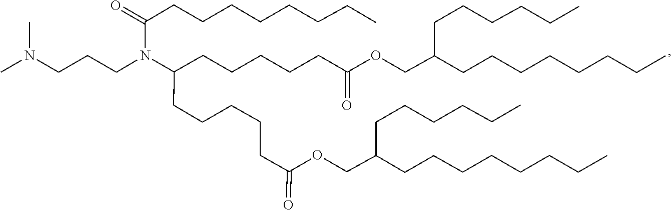 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof.

In some embodiments, the RNA composition (e.g., the nucleic acid vaccine composition) and methods for making and using thereof include a lipid having the compound structure:

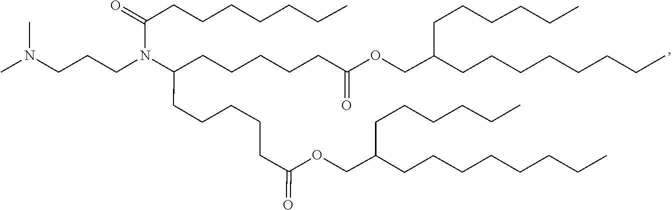 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof.

In some embodiments, the RNA composition (e.g., the nucleic acid vaccine composition) and methods for making and using thereof include a lipid having the compound structure:

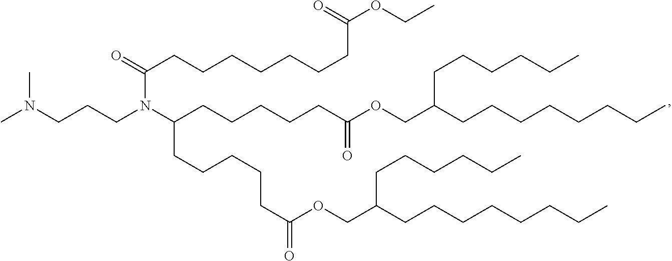 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof.

In some embodiments, the RNA composition (e.g., the nucleic acid vaccine composition) and methods for making and using thereof include a lipid having the compound structure:

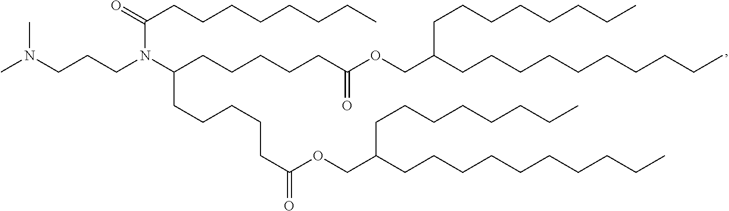 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable lipids for use in the RNA composition (e.g., the nucleic acid vaccine composition) and methods for making and using thereof include the lipids as described in International Patent Publication WO 2017/075531, which is incorporated herein by reference in its entirety.

In some embodiments, the RNA composition (e.g., the nucleic acid vaccine composition) and methods for making and using thereof include a lipid of the following formula:

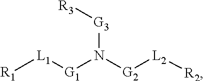

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

Other suitable lipids for use in the RNA composition (e.g., the nucleic acid vaccine composition) and methods for making and using thereof include the lipids as described in International Patent Publication WO 2017/117528, which is incorporated herein by reference in its entirety. In some embodiments, the RNA composition (e.g., the nucleic acid vaccine composition) and methods for making and using thereof include a lipid having the compound structure:

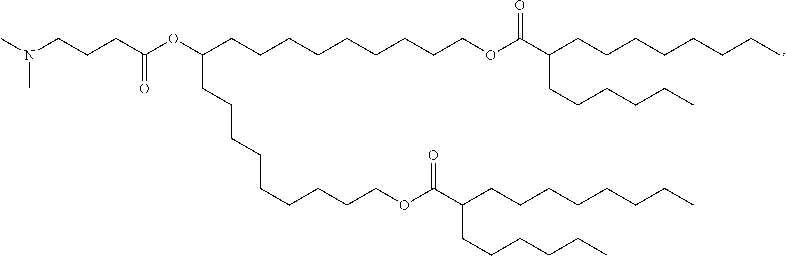 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof.

In some embodiments, the RNA composition (e.g., the nucleic acid vaccine composition) and methods for making and using thereof include a lipid having the compound structure:

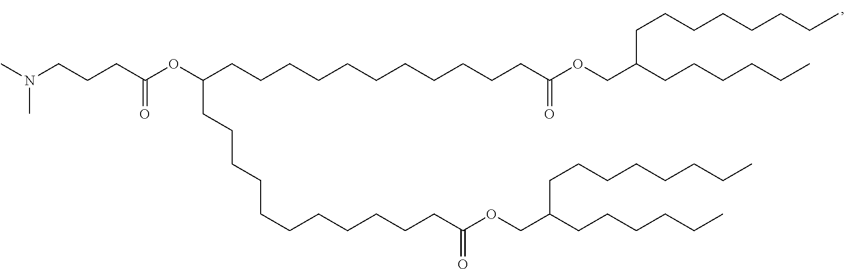 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof.

In some embodiments, the RNA composition (e.g., the nucleic acid vaccine composition) and methods for making and using thereof include a lipid having the compound structure:

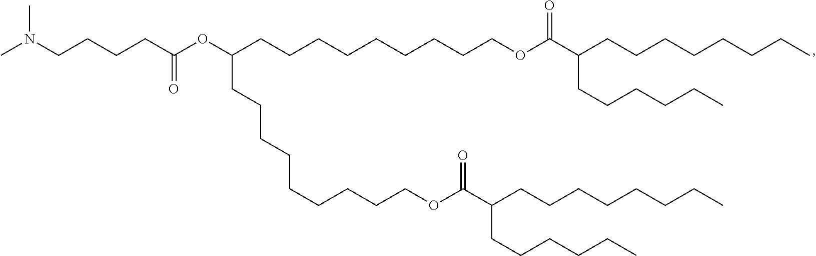 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable lipids for use in the RNA composition (e.g., the nucleic acid vaccine composition) and methods for making and using thereof include the lipids as described in International Patent Publication WO 2017/049245, which is incorporated herein by reference in its entirety.

In some embodiments, the lipids of the RNA composition (e.g., the nucleic acid vaccine composition) and methods for making and using thereof include a compound of the following formulas:

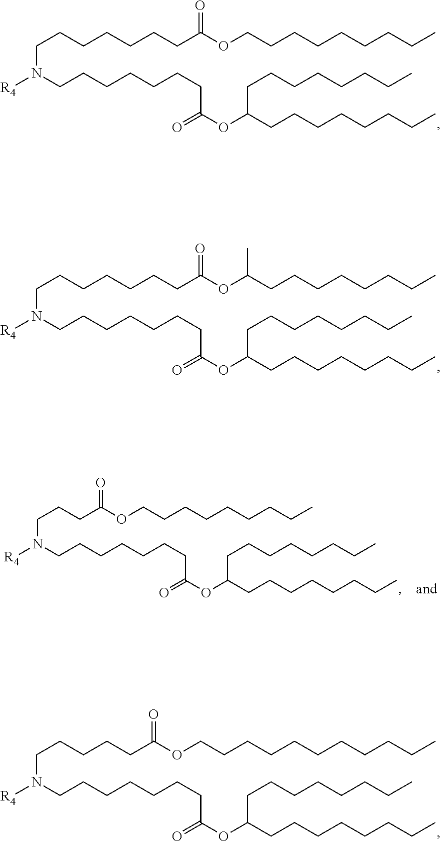

and pharmaceutically acceptable salts thereof. For any one of these four formulas, R₄ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3.

In certain embodiments, the RNA composition (e.g., the nucleic acid vaccine composition) and methods for making and using thereof include a lipid having a compound structure of:

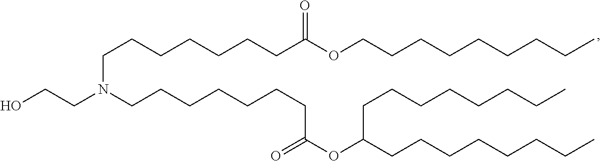 [SMILES: CCCCCCCCCOC(=O)CCCCCCCN(CCO)CCCCCCCC(=O)OC(CCCCCCCC)CCCCCCCC]

and pharmaceutically acceptable salts thereof.

In certain embodiments, the RNA composition (e.g., the nucleic acid vaccine composition) and methods for making and using thereof include a lipid having a compound structure of:

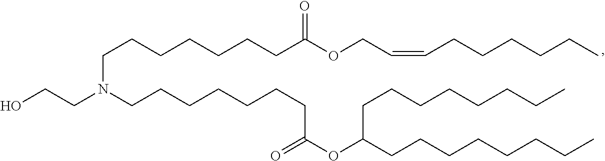 [SMILES: CCCCCC/C=C\COC(=O)CCCCCCCN(CCO)CCCCCCCC(=O)OC(CCCCCCCC)CCCCCCCC]

and pharmaceutically acceptable salts thereof.

In certain embodiments, the RNA composition (e.g., the nucleic acid vaccine composition) and methods for making and using thereof include a lipid having a compound structure of:

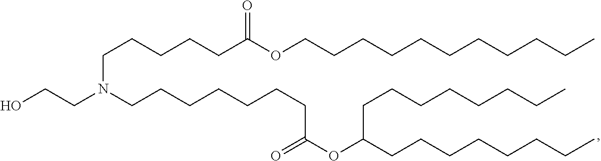 [SMILES: CCCCCCCCCCCOC(=O)CCCCCN(CCO)CCCCCCCC(=O)OC(CCCCCCCC)CCCCCCCC ; ","]

and pharmaceutically acceptable salts thereof.

In certain embodiments, the RNA composition (e.g., the nucleic acid vaccine composition) and methods for making and using thereof include a lipid having a compound structure of:

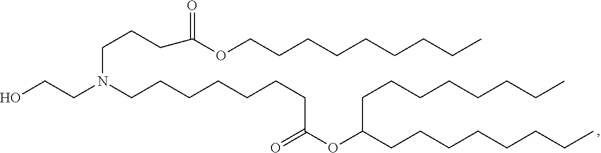 [SMILES: CCCCCCCCCOC(=O)CCCN(CCO)CCCCCCCC(=O)OC(CCCCCCCC)CCCCCCCC]

and pharmaceutically acceptable salts thereof.

Other suitable lipids for use in the RNA composition (e.g., the nucleic acid vaccine composition) and methods for making and using thereof include the lipids as described in International Patent Publication WO 2017/173054 and WO 2015/095340, each of which is incorporated herein by reference in its entirety. In certain embodiments, the RNA composition (e.g., the nucleic acid vaccine composition) and methods for making and using thereof include a lipid having a compound structure of:

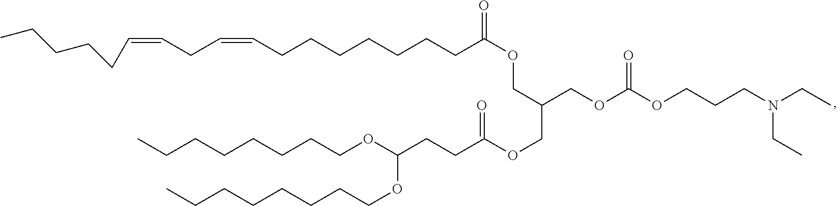 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof.

In certain embodiments, the RNA composition (e.g., the nucleic acid vaccine composition) and methods for making and using thereof include a lipid having a compound structure of:

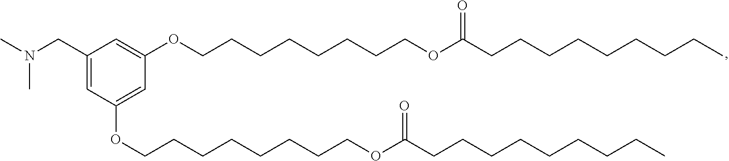 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof.

In certain embodiments, the RNA composition (e.g., the nucleic acid vaccine composition) and methods for making and using thereof include a lipid having a compound structure of:

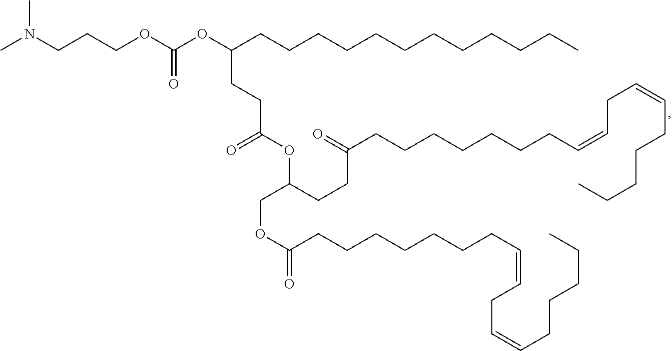 [SMILES: CCCCC/C=C\C/C=C\CCCCCCCC(=O)CCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof.

In certain embodiments, the RNA composition (e.g., the nucleic acid vaccine composition) and methods for making and using thereof include a lipid having a compound structure of:

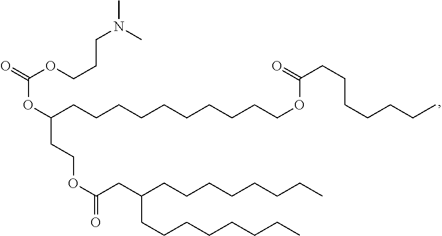 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

In some embodiments, the LNMPs described herein may include a ionizable lipid as described in, may be formulated as described in, or may comprise or be comprised by a composition as described in WO2016118724, WO2016118725, WO2016187531, WO2017176974, WO2018078053, WO2019027999, WO2019036030, WO2019089828, WO2019099501, WO2020072605, WO2020081938, WO2020118041, WO2020146805, or WO2020219876, each of which is incorporated by reference herein in its entirety.

The ionizable lipids disclosed herein may be used to form the LNMP composition together with one or more natural lipids disclosed herein. In some embodiments, the LNMP composition is formulated to further comprise one or more therapeutic agents. In some embodiments, the LNMP composition is a lipid nanoparticle that encapsulates or is associated with the one or more nucleic acid vaccines.

In some embodiments, the nucleic acid vaccine disclosed herein has an N/P ratio of at least 3, for instance, an N/P ratio of 3 to 100, 3 to 50, 3 to 30, 3 to 20, 3 to 15, 3 to 12, 3 to 10, 6 to 30, 6 to 20, 6 to 15, or 6 to 12. For example, the N/P ratio may be 6±1, or the N/P ratio may be 6±0.5. In some embodiments, the N/P ratio is about 6. In some embodiments, the N/P ratio is about 3 (e.g., 3±1 or 3±0.5). In some embodiments, the nucleic acid vaccine composition has an N/P ratio of about 12 to about 17, for example, the N/P ratio is about 15±1, or the N/P ratio is about 15±0.5. In some embodiments, the N/P ratio is about 15. In some embodiments, the N/P ratio is about 12 (e.g., 12±1 or 12±0.5).

In some embodiments, the disclosure relates to a composition comprising (i) one or more compounds chosen from the ionizable lipids of Formula (I)—(III), pharmaceutically acceptable salts thereof, and stereoisomers of any of the foregoing and (ii) a lipid component. In some embodiments, the composition comprises 5%, 10%, 15%, 20%, 25%, 30%, 35%, 40%, 45%, 50%, 55%, 60%, 65%, 70%, 75%, 80%, 85%, 90%, or 95% of the one or more compounds.

In some embodiments, the disclosure relates to a composition comprising (i) one or more lipid nanoparticles and (ii) one or more lipid components.

In some embodiments, one or more lipid components comprise one or more helper lipids and one or more PEG lipids. In some embodiments, the lipid component(s) comprise(s) one or more helper lipids, one or more PEG lipids, and one or more neutral lipids.

Non-limiting examples of neutral lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids may be acyl groups derived from fatty acids having C₁₀-C₂₄ carbon chains, e.g., lauroyl, myristoyl, palmitoyl, stearoyl, or oleoyl.

In some embodiments, the nucleic acid vaccine comprises a phytosterol or a combination of a phytosterol and cholesterol. In some embodiments, the phytosterol is selected from the group consisting of b-sitosterol, stigmasterol, b-sitostanol, campesterol, brassicasterol, and combinations thereof. In some embodiments, the phytosterol is selected from the group consisting of b-sitosterol, b-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof. In some embodiments, the phytosterol is selected from the group consisting of Compound S-140, Compound S-151, Compound S-156, Compound S-157, Compound S-159, Compound S-160, Compound S-164, Compound S-165, Compound S-170, Compound S-173, Compound S-175, and combinations thereof. In some embodiments, the phytosterol is a combination of Compound S-141, Compound S-140, Compound S-143 and Compound S-148. In some embodiments, the phytosterol comprises a sitosterol or a salt or an ester thereof. In some embodiments, the phytosterol comprises a stigmasterol or a salt or an ester thereof.

### Other Lipids and Other Agents

The exogenous lipid may be a cell-penetrating agent, may be capable of increasing delivery of a polypeptide by the LNMP to a cell, and/or may be capable of increasing loading (e.g., loading efficiency or loading capacity) of a polypeptide. Further exemplary exogenous lipids include sterols and PEGylated lipids.

The LNMPs can be modified with other components (e.g., lipids, e.g., sterols, e.g., cholesterol; or small molecules) to further alter the functional and structural characteristics of the LNMP. For example, the LNMPs can be further modified with stabilizing molecules that increase the stability of the LNMPs (e.g., for at least one day at room temperature, and/or stable for at least one week at 4° C.).

In some embodiments, the LNMP is modified with a sterol, e.g., sitosterol, sitostanol, β-sitosterol, 7α-hydroxycholesterol, pregnenolone, cholesterol (e.g., ovine cholesterol or cholesterol isolated from plants), stigmasterol, campesterol, fucosterol, or an analog (e.g., a glycoside, ester, or peptide) of any sterol. In some examples, the exogenous sterol is added to the preparation prior to step (b), e.g., mixed with extracted NMP lipids prior to step (b). The exogenous sterol may be added to amount to, e.g., 1%, 5%, 10%, 20%, 30%, 40%, 50%, 60%, 70%, 80%, 90%, or more than 90% (w/w) of total lipids and sterols in the preparation.

In some embodiments, the sterol is cholesterol or sitosterol. In some instances, the LNMPs comprise a molar ratio of least 1%, 5%, 10%, 15%, 20%, 25%, 30%, 35%, 40%, 45%, 50%, 55%, 60%, or more than 60% sterol (e.g., cholesterol or sitosterol), e.g., 1%-10%, 10%-20%, 20%-30%, 30%-40%, 40%-50%, or 50%-60% sterol. In some embodiments, the LNMP comprises a molar ratio of about 35%-50% sterol (e.g., cholesterol or sitosterol), e.g., about 36%, 38.5%, 42.5%, or 46.5% sterol. In some embodiments, the LNMP comprises a molar ratio of about 20%-40% sterol.

In some embodiments, a LNMP that has been modified with a sterol has altered stability (e.g., increased stability) relative to a LNMP that has not been modified with a sterol. In some aspects, a LNMP that has been modified with a sterol has a greater rate of fusion with a membrane of a target cell relative to a LNMP that has not been modified with a sterol.

In some instances, the LNMPs comprise an exogenous lipid and an exogenous sterol.

In some embodiments, the LNMP is modified with a PEGylated lipid. Polyethylene glycol (PEG) length can vary from 1 kDa to 10 kDa; in some aspects, PEG having a length of 2 kDa is used. In some embodiments, the PEGylated lipid is C14-PEG2k, C18-PEG2k, or DMPE-PEG2k. In some instances, the LNMPs comprise a molar ratio of at least 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.5%, 4%, 4.5%, 5%, 10%, 20%, 30%, 40%, 50%, or more than 50% PEGylated lipid (e.g., C14-PEG2k, C18-PEG2k, or DMPE-PEG2k), e.g., 0.1%-0.5%, 0.5%-1%, 1%-1.5%, 1.5%-2.5%, 2.5%-3.5%, 3.5%-5%, 5%-10%, 10%-20%, 20%-30%, 30%-40%, or 30%-50% PEGylated lipid. In some embodiments, the LNMP comprises a molar ratio of about 0.1%-10% PEGylated lipid (e.g., C14-PEG2k, C18-PEG2k, or DMPE-PEG2k), e.g., about 1%-3% PEGylated lipid, e.g., about 1.5% or about 2.5% PEGylated lipid. In some embodiments, a LNMP that has been modified with a PEGylated lipid has altered stability (e.g., increased stability) relative to a LNMP that has not been modified with a PEGylated lipid. In some embodiments, a LNMP that has been modified with a PEGylated lipid has altered particle size relative to a LNMP that has not been modified with a PEGylated lipid. In some embodiments, a LNMP that has been modified with a PEGylated lipid is less likely to be phagocytosed than a LNMP that has not been modified with a PEGylated lipid. The addition of PEGylated lipids can also affect stability in GI tract and enhance particle migration through mucus. PEG may be used as a method to attach targeting moieties.

In some embodiments, the LNMPs are modified with an ionizable lipid (e.g., C12-200 or MC3) and one or both of a sterol (e.g., cholesterol or sitosterol) and a PEGylated lipid (e.g., C14-PEG2k, C18-PEG2k, or DMPE-PEG2k).

In some embodiments, the modified LNMPs comprise a molar ratio of about 5%-50% LNMP lipids (e.g., about 10%-20% LNMP lipids, e.g., about 10%, 12.5%, 16%, or 20% LNMP lipids); about 30%-75% ionizable lipids (e.g., about 35% or about 50% ionizable lipids); about 35%-50% sterol (e.g., about 36%, 38.5%, 42.5%, or 46.5% sterol); and about 0.1%-10% PEGylated lipid (e.g., about 1%-3% PEGylated lipid, e.g., about 1.5% or about 2.5% PEGylated lipid).

In some embodiments, the modified LNMPs comprise a molar ratio of about 5%-60% LNMP lipids (e.g., about 10%-20%, 20%-30%, 30%-40%, 40%-50%, or 50%-60% LNMP lipids, e.g., about 10%, 12.5%, 16%, 20%, 30%, 40%, 50%, or 60% LNMP lipids); about 25%-75% ionizable lipids (e.g., about 35% or about 50% ionizable lipids); about 10%-50% sterol (e.g., about 10%, 12.5%, 14%, 16%, 18%, 20%, 36%, 38.5%, 42.5%, or 46.5% sterol); and about 0.1%-10% PEGylated lipid (e.g., about 0.5%-5% PEGylated lipid, e.g., about 1%-3% PEGylated lipid, or about 1.5% or about 2.5% PEGylated lipid).

In some embodiments, the ionizable lipids, LNMP lipids, sterol, and PEGylated lipid comprise about 25%-75%, about 20%-60%, about 10%-45%, and about 0.5%-5%, respectively, of the lipids in the modified LNMP.

In some embodiments, the ionizable lipids, natural lipids, sterol, and PEGylated lipid comprise about 30%-75%, about 20%-50%, about 10%-45%, and about 1%-5%, respectively, of the lipids in the modified LNMP.

In some embodiments, the ionizable lipids, natural lipids, sterol, and PEGylated lipid comprise about 35%-75%, about 20%-50%, about 10%-45%, and about 1%-5%, respectively, of the lipids in the modified LNMP.

In some embodiments, the ionizable lipids, natural lipids, sterol, and PEGylated lipid are formulated at a molar ratio of about 35:50:12.5:2.5.

In some embodiments, the ionizable lipids, natural lipids, sterol, and PEGylated lipid are formulated at a molar ratio of about 35:50:11.5:3.5.

In some embodiments, the ionizable lipids, natural lipids, sterol, and PEGylated lipid are formulated at a molar ratio of about 35:20:42.5:2.5.

In some embodiments, a LNMP has been modified with an ionizable lipid (and/or cationic lipid) and a sterol and/or a PEGylated lipid more efficiently encapsulates a negatively charged cargo (e.g., a nucleic acid) than a LNMP that has not been modified with an ionizable lipid (and/or cationic lipid) and a sterol and/or a PEGylated lipid. The modified LNMP may have an encapsulation efficiency for the cargo (e.g., nucleic acid, e.g., RNA or DNA) that is at least 5%, 10%, 15%, 20%, 30%, 40%, 50%, 60%, 70%, 80%, 90%, 95%, 98%, 99%, or more than 99%, e.g., may have an encapsulation efficiency of 5%-30%, 30%-50%, 50%-70%, 70%-80%, 80%-90%, 90%-95%, or 95%-100%.

Cell uptake of the modified LNMPs can be measured by a variety of methods known in the art. For example, the LNMPs, or a component thereof, can be labelled with a marker (e.g., a fluorescent marker) that can be detected in isolated cells to confirm uptake.

In some embodiments, a LNMP formulation provided herein comprises two or more different modified LNMPs, e.g., comprises modified LNMPs derived from different unmodified LNMPs (e.g., unmodified LNMPs from two or more different natural sources) and/or comprises modified LNMPs comprising different species and/or different ratios of ionizable lipids, sterols, and/or PEGylated lipids.

In some instances, the organic solvent in which the lipid film is dissolved is dimethylformamide:methanol (DMF:MeOH). Alternatively, the organic solvent or solvent combination may be, e.g., acetonitrile, acetone, ethanol, methanol, dimethylformamide, tetrahydrofuran, 1-buthanol, dimethyl sulfoxide, acetonitrile:ethanol, acetonitrile:methanol, acetone:methanol, methyl tert-butyl ether:propanol, tetrahydrofuran:methanol, dimethyl sulfoxide:methanol, or dimethylformamide:methanol.

The aqueous phase may be any suitable solution, e.g., a citrate buffer (e.g., a citrate buffer having a pH of about 3.2), water, or phosphate-buffered saline (PBS). The aqueous phase may further comprise a nucleic acid (e.g., an siRNA or siRNA precursor (e.g., dsRNA), miRNA or miRNA precursor, mRNA, circRNA, or plasmid (pDNA)) or a small molecule.

The lipid solution and the aqueous phase may be mixed in the microfluidics device at any suitable ratio. In some examples, aqueous phase and the lipid solution are mixed at a 3:1 volumetric ratio.

LNMPs may optionally include additional agents, e.g., cell-penetrating agents, therapeutic agents, polynucleotides, polypeptides, or small molecules. The LNMPs can carry or associate with additional agents in a variety of ways to enable delivery of the agent to a target plant or animal, e.g., by encapsulating the agent, incorporation of the agent in the lipid bilayer structure, or association of the agent (e.g., by conjugation) with the surface of the lipid bilayer structure. Nucleic acid molecules can be incorporated into the LNMPs either in vivo or in vitro (e.g., in tissue culture, in cell culture, or synthetically incorporated).

### Zeta Potential

The LNMPs comprising an ionizable lipid and optionally a cationic lipid (e.g., DC-cholesterol or DOTAP) may have, e.g., a zeta potential of greater than −30 mV when in the absence of cargo, greater than −20 mV, greater than −5 mV, greater than 0 mV, or about 30 my when in the absence of cargo. In some examples, the LNMP has a negative zeta potential, e.g., a zeta potential of less than 0 mV, less than −10 mV, less than −20 mV, less than −30 mV, less than −40 mV, or less than −50 mV when in the absence of cargo. In some examples, the LNMP has a positive zeta potential, e.g., a zeta potential of greater than 0 mV, greater than 10 mV, greater than 20 mV, greater than 30 mV, greater than 40 mV, or greater than 50 mV when in the absence of cargo. In some examples, the LNMP has a zeta potential of about 0.

The zeta potential of the LNMP may be measured using any method known in the art. Zeta potentials are generally measured indirectly, e.g., calculated using theoretical models from the data obtained using methods and techniques known in the art, e.g., electrophoretic mobility or dynamic electrophoretic mobility. Electrophoretic mobility is typically measured using microelectrophoresis, electrophoretic light scattering, or tunable resistive pulse sensing. Electrophoretic light scattering is based on dynamic light scattering. Typically, zeta potentials are accessible from dynamic light scattering (DLS) measurements, also known as photon correlation spectroscopy or quasi-elastic light scattering.

### Plant EV-Markers

The LNMPs in the nucleic acid vaccine and methods of making and using thereof may have a range of markers that identify the LPMPs as being produced using a plant EV, and/or including a segment, portion, or extract thereof. As used herein, the term “plant EV-marker” refers to a component that is naturally associated with a plant and incorporated into or onto the plant EV in planta, such as a plant protein, a plant nucleic acid, a plant small molecule, a plant lipid, or a combination thereof. Examples of plant EV-markers can be found, for example, in Rutter and Innes, Plant Physiol. 173(1): 728-741, 2017; Raimondo et al., Oncotarget. 6(23): 19514, 2015; Ju et al., Mol. Therapy. 21(7):1345-1357, 2013; Wang et al., Molecular Therapy. 22(3): 522-534, 2014; and Regente et al, J of Exp. Biol. 68(20): 5485-5496, 2017; each of which is incorporated herein by reference.

Additional examples of the suitable plant EV-markers include those described and listed in International Patent Application Publication No. WO 2021/041301, which is incorporated herein by reference in its entirety.

### Bacterial EV-Markers

The bacterial components (e.g., bacterial lipids) in the bacteria-derived lipid composition and methods of making and using thereof may have a range of markers that identify the bacterial component as being produced. As used herein, the term “bacterial EV-marker” refers to a component that is naturally associated with a bacterium and incorporated into or onto the bacterial EV, such as a bacterial protein, a bacterial nucleic acid, a bacterial small molecule, a bacterial lipid, or a combination thereof.

### Natural Source EV-Markers

The NMPs may have a range of markers that identify the NMP as being produced from a specific source EV, and/or including a segment, portion, or extract thereof. As used herein, the term “EV-marker” refers to a component that is naturally associated with a specific source and incorporated into or onto the EV in vivo, such as a protein, a nucleic acid, a small molecule, a lipid, or a combination thereof. Examples of source EV-markers include but are not limited to peptidoglycan, lipopolysaccharide, ester-linked lipids, ether-linked lipids, circular DNA, chitin, beta-glucan, pekilo, mycoprotein, cerato-platanins, exotoxins, diacylglycerol, triglycerides, phosphatidylcholine, phosphatidylinositol, ornithine lipids, glycolipids, sphingolipids, hopanoids, or ergosterol.

The source EV marker can include a lipid. Examples of lipid markers that may be found in the NMP include lipid A, lipopolysaccharide, ergosterol, ornithine lipids (OLs), sulfolipids, diacylglyceryl-N,N,N-trimethylhomoserine (DGTS), glycolipids (GLs), diacylglycerol (DAG), hopanoids (HOPs), glucosyleramide, sterylglycosides, ether-linked lipids, or a combination thereof.

Other EV markers may include lipids that accumulate in sources in response to abiotic or biotic stressors.

Alternatively, the source EV marker may include a protein. In some instances, the protein EV marker may be an antimicrobial or antiviral protein naturally produced by the source, including proteins that are secreted in response to abiotic or biotic stressors. Some examples of protein EV markers include but are not limited to cecropins, moricins, defensins, proline- and glycine-rich peptides, fungal immunomodulatory proteins, flagellin, encapsulin, streptavidin, internalin, pilin, halocin, or archaeocins. In some instances, the EV marker can include a protein involved in lipid metabolism. In some instances, the protein EV marker is a cellular trafficking protein in the source. In certain instances where the EV marker is a protein, the protein marker may lack a signal peptide that is typically associated with secreted proteins. Unconventional secretory proteins seem to share several common features like (i) lack of a leader sequence, (ii) absence of PTMs specific for ER or Golgi apparatus, and/or (iii) secretion not affected by brefeldin A which blocks the classical ER/Golgi-dependent secretion pathway. One skilled in the art can use a variety of tools freely accessible to the public to evaluate a protein for a signal sequence, or lack thereof.

In instances where the EV marker is a protein, the protein may have an amino acid sequence having at least 35%, 40%, 45%, 50%, 55%, 60%, 65%, 70%, 75%, 80%, 85%, 90%, 95%, 98%, 99%, or 100% sequence identity to a known EV marker.

In some instances, the EV marker includes a nucleic acid encoded in the source, e.g., an Arthropod, Plant, Fungi, Archaea, or Bacteria RNA, DNA, or PNA. For example, the NMP may include dsRNA, mRNA, circRNA, a viral RNA, a microRNA (miRNA), or a small interfering RNA (siRNA) encoded by the source. In some instances, the nucleic acid may be one that is associated with a protein that facilitates the long-distance transport of RNA. In some instances, the nucleic acid EV marker may be one involved in host-induced gene silencing (HIGS), which is the process by which a source silences foreign transcripts of pathogens. In some instances, the nucleic acid may be a microRNA.

In instances where the EV marker is a nucleic acid, the nucleic acid may have a nucleotide sequence having at least 35%, 40%, 45%, 50%, 55%, 60%, 65%, 70%, 75%, 80%, 85%, 90%, 95%, 98%, 99%, or 100% sequence identity to a known EV marker.

In some instances, the EV marker includes a compound produced by the source. For example, the compound may a component of the cell wall (e.g. lipopolysaccharide). For example, the compound may be a defense compound produced in response to abiotic or biotic stressors, such as pathogens or extreme environmental stress.

In some instances, the NMP may also be identified as being produced from a source EV based on the lack of certain markers (e.g., lipids, polypeptides, or polynucleotides) that are not typically produced by these sources, but are generally associated with other organisms (e.g., markers of animal EVs or plant EVs). For example, in some instances, the NMP lacks lipids typically found in animal EVs or plant EVs.

EV markers can be identified using any approaches known in the art that enable identification of small molecules (e.g., mass spectroscopy, mass spectrometry), lipids (e.g., mass spectroscopy, mass spectrometry), proteins (e.g., mass spectroscopy, immunoblotting), or nucleic acids (e.g., PCR analysis). In some instances, a NMP composition described herein includes a detectable amount, e.g., a pre-determined threshold amount, of an EV marker described herein.

### Loading of Agents (e.g., Nucleic Acids)

The LNMPs are modified to include a therapeutic agent (e.g., a nucleic acid molecule) to form the nucleic acid vaccine. The LNMPs can carry or associate with such agents by a variety of means to enable delivery of the agent to a target organism (e.g., a target animal), e.g., by encapsulating the agent, incorporation of the component in the lipid bilayer structure, or association of the component (e.g., by conjugation) with the surface of the lipid bilayer structure of the LNMP. In some instances, the agent is included in the LNMP formulation, as described herein.

The agent can be incorporated or loaded into or onto the LNMPs by any methods known in the art that allow association, directly or indirectly, between the LNMPs and agent. The agents can be incorporated into the LNMPs by an in vivo method (e.g., in planta, e.g., through production of LNMPs from a transgenic plant that comprises the agent), or in vitro (e.g., in tissue culture, or in cell culture), or both in vivo and in vitro methods.

In some instances, the LNMPs are loaded in vitro. The substance may be loaded onto or into (e.g., may be encapsulated by) the LNMPs using, but not limited to, physical, chemical, and/or biological methods (e.g., in tissue culture or in cell culture). For example, the agent may be introduced into LNMPs by one or more of electroporation, sonication, passive diffusion, stirring, lipid extraction, or extrusion. In some instances, the agent is incorporated into the LNMP using a microfluidic device, e.g., using a method in which LNMP lipids are provided in an organic phase, the heterologous functional agent is provided in an aqueous phase, and the organic and aqueous phases are combined in the microfluidics device to produce a LNMP comprising the heterologous functional agent. Loaded LNMPs can be assessed to confirm the presence or level of the loaded agent using a variety of methods, such as HPLC (e.g., to assess small molecules), immunoblotting (e.g., to assess proteins); and/or quantitative PCR (e.g., to assess nucleotides). However, it should be appreciated by those skilled in the art that the loading of a substance of interest into LNMPs is not limited to the above-illustrated methods.

In some instances, the agent can be conjugated to the LNMP, in which the agent is connected or joined, indirectly or directly, to the LNMP. For instance, one or more agents can be chemically linked to a LNMP, such that the one or more agents are joined (e.g., by covalent or ionic bonds) directly to the lipid bilayer of the LNMP. In some instances, the conjugation of various agents to the LNMPs can be achieved by first mixing the one or more agents with an appropriate cross-linking agent (e.g., N-ethylcarbo-diimide (“EDC”), which is generally utilized as a carboxyl activating agent for amide bonding with primary amines and also reacts with phosphate groups) in a suitable solvent. After a period of incubation sufficient to allow the agent to attach to the cross-linking agent, the cross-linking agent/agent mixture can then be combined with the LNMPs and, after another period of incubation, subjected to a sucrose gradient (e.g., and 8, 30, 45, and 60% sucrose gradient) to separate the free agent and free LNMPs from the agent conjugated to the LNMPs. As part of combining the mixture with a sucrose gradient, and an accompanying centrifugation step, the LNMPs conjugated to the agent are then seen as a band in the sucrose gradient, such that the conjugated LNMPs can then be collected, washed, and dissolved in a suitable solution for use as described herein.

In some instances, the LNMPs are stably associated with the agent prior to and following delivery of the LNMP, e.g., to a plant or animal. In other instances, the LNMPs are associated with the agent such that the agent becomes dissociated from the LNMPs following delivery of the LNMP, e.g., to a plant or animal.

The LNMPs can be loaded or the LNMP can be formulated with various concentrations of the agent, depending on the particular agent or use. For example, in some instances, the LNMPs are loaded or the LNMP is formulated such that the LNMP formulation disclosed herein includes about 0.001, 0.01, 0.1, 1.0, 2, 3, 4, 5, 6, 7, 8, 9, 10, 15, 20, 30, 40, 50, 60, 70, 80, 90, or 95 (or any range between about 0.001 and 95) or more wt % of an agent. In some instances, the LNMPs are loaded or the LNMP is formulated such that the LNMP formulation includes about 95, 90, 80, 70, 60, 50, 40, 30, 20, 15, 10, 9, 8, 7, 6, 5, 4, 3, 2, 1.0, 0.1, 0.01, 0.001 (or any range between about 95 and 0.001) or less wt % of an agent. For example, the LNMP formulation can include about 0.001 to about 0.01 wt %, about 0.01 to about 0.1 wt %, about 0.1 to about 1 wt %, about 1 to about 5 wt %, or about 5 to about 10 wt %, about 10 to about 20 wt % of the agent. In some instances, the LNMP can be loaded or the LNMP is formulated with about 1, 5, 10, 50, 100, 200, or 500, 1,000, 2,000 (or any range between about 1 and 2,000) or more μg/ml of an agent. A LNMP of the invention can be loaded or a LNMP can be formulated with about 2,000, 1,000, 500, 200, 100, 50, 10, 5, 1 (or any range between about 2,000 and 1) or less μg/ml of an agent.

In some instances, the LNMPs are loaded or the LNMP is formulated such that the LNMP formulation disclosed herein includes at least 0.001 wt %, at least 0.01 wt %, at least 0.1 wt %, at least 1.0 wt %, at least 2 wt %, at least 3 wt %, at least 4 wt %, at least 5 wt %, at least 6 wt %, at least 7 wt %, at least 8 wt %, at least 9 wt %, at least 10 wt %, at least 15 wt %, at least 20 wt %, at least 30 wt %, at least 40 wt %, at least 50 wt %, at least 60 wt %, at least 70 wt %, at least 80 wt %, at least 90 wt %, or at least 95 wt % of an agent. In some instances, the LNMP can be loaded or the LNMP can be formulated with at least 1 μg/ml, at least 5 μg/ml, at least 10 μg/ml, at least 50 μg/ml, at least 100 μg/ml, at least 200 μg/ml, at least 500 μg/ml, at least 1,000 μg/ml, at least 2,000 μg/ml of an agent.

In some instances, the LNMP is formulated with the agent by suspending the LNMPs in a solution comprising or consisting of the agent, e.g., suspending or resuspending the LNMPs by vigorous mixing. The agent (e.g., cell-penetrating agent, e.g., nucleic acids, enzyme, detergent, ionic, fluorous, or zwitterionic liquid, or ionizable lipid may comprise, e.g., less than 1% or at least 1%, 5%, 10%, 20%, 30%, 40%, 50%, 60%, 70%, 80%, 90%, or 100% of the solution.

### Pharmaceutical Formulations

The modified LNMPs are formulated into pharmaceutical compositions (i.e., a nucleic acid vaccine composition), e.g., for administration to an animal (e.g., a human). The pharmaceutical composition may be administered to an animal (e.g., human) with a pharmaceutically acceptable diluent, carrier, and/or excipient. Depending on the mode of administration and the dosage, the pharmaceutical composition of the methods described herein will be formulated into suitable pharmaceutical compositions to permit facile delivery. The single dose may be in a unit dose form as needed.

In some embodiments, the dose is 0.005 mg/kg, 0.01 mg/kg, 0.05 mg/kg, 0.1 mg/kg, 0.2 mg/kg, 0.3 mg/kg, 0.4 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, or more.

In some embodiments, the vaccine is administered once, twice, three times, four times, or more.

The LNMP/nucleic acid vaccine may be formulated for e.g., oral administration, intranasal, intravenous administration (e.g., injection or infusion), intramuscular, or subcutaneous administration to an animal. For injectable formulations, various effective pharmaceutical carriers are known in the art (See, e.g., Remington: The Science and Practice of Pharmacy, 22^{nd} ed., (2012) and ASHP Handbook on Injectable Drugs, 18^{th} ed., (2014)).

Suitable pharmaceutically acceptable carriers and excipients are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. The LNMP/nucleic acid vaccine may be formulated according to conventional pharmaceutical practice. The concentration of the compound in the formulation will vary depending upon a number of factors, including the dosage of the active agent (e.g., LNMPs and nucleic acids) to be administered, and the route of administration.

For oral administration to an animal, the LNMP/nucleic acid vaccine can be prepared in the form of an oral formulation. Formulations for oral use can include tablets, caplets, capsules, syrups, or oral liquid dosage forms containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Formulations for oral use may also be provided in unit dosage form as chewable tablets, non-chewable tablets, caplets, capsules (e.g., as hard gelatin capsules wherein the active ingredient is mixed with an inert solid diluent, or as soft gelatin capsules wherein the active ingredient is mixed with water or an oil medium). The compositions disclosed herein may also further include an immediate-release, extended release or delayed-release formulation.

For parenteral administration to an animal, the LNMP/nucleic acid vaccines may be formulated in the form of liquid solutions or suspensions and administered by a parenteral route (e.g., subcutaneous, intravenous, or intramuscular). The pharmaceutical composition can be formulated for injection or infusion. Pharmaceutical compositions for parenteral administration can be formulated using a sterile solution or any pharmaceutically acceptable liquid as a vehicle. Pharmaceutically acceptable vehicles include, but are not limited to, sterile water, physiological saline, or cell culture media (e.g., Dulbecco's Modified Eagle Medium (DMEM), α-Modified Eagles Medium (α-MEM), and F-12 medium). Formulation methods are known in the art, see e.g., Gibson (ed.) Pharmaceutical Preformulation and Formulation (2nd ed.) Taylor & Francis Group, CRC Press (2009).

### Polynucleotides

The LNMP/nucleic acid vaccine includes one or more nucleic acid molecules, e.g., polynucleotides, which encode one or more wild type or engineered proteins, peptides, or polypeptides. Exemplary polynucleotides, e.g., polynucleotide constructs, include antigen-encoding RNA polynucleotides, e.g., mRNAs, linear polyribonucleotides, or circRNAs.

Examples of polypeptides that can be used herein can include an enzyme (e.g., a metabolic recombinase, a helicase, an integrase, a RNAse, a DNAse, or an ubiquitination protein), a pore-forming protein, a signaling ligand, a cell penetrating peptide, a transcription factor, a receptor, an antibody, a nanobody, a gene editing protein (e.g., CRISPR-Cas system, TALEN, or zinc finger), riboprotein, a protein aptamer, or a chaperone.

Polypeptides included herein may include naturally occurring polypeptides or recombinantly produced variants. In some instances, the polypeptide may be a functional fragments or variants thereof (e.g., an enzymatically active fragment or variant thereof). For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide may have at least 50% (e.g., at least 50%, 60%, 70%, 80%, 90%, 95%, 97%, 99%, or greater) identity to a protein of interest.

The polypeptide may have a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less may be useful.

The LNMP/nucleic acid vaccine may include any number or type (e.g., classes) of polypeptides, such as at least about any one of 1 polypeptide, 2, 3, 4, 5, 10, 15, 20, or more polypeptides. A suitable concentration of each polypeptide in the LNMP/nucleic acid vaccine depends on factors such as efficacy, stability of the polypeptide, number of distinct polypeptides in the formulation, and methods of application of the formulation. In some instances, each polypeptide in a liquid formulation is from about 0.1 ng/mL to about 100 mg/mL. In some instances, each polypeptide in a solid formulation is from about 0.1 ng/g to about 100 mg/g.

### Nucleic Acids Encoding Peptides

In some instances, the LNMP/nucleic acid vaccine include a heterologous nucleic acid encoding a polypeptide. Nucleic acids encoding a polypeptide may have a length from about 10 to about 50,000 nucleotides (nts), about 25 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, about 5000 to about 6000 nts, about 6000 to about 7000 nts, about 7000 to about 8000 nts, about 8000 to about 9000 nts, about 9000 to about 10,000 nts, about 10,000 to about 15,000 nts, about 10,000 to about 20,000 nts, about 10,000 to about 25,000 nts, about 10,000 to about 30,000 nts, about 10,000 to about 40,000 nts, about 10,000 to about 45,000 nts, about 10,000 to about 50,000 nts, or any range therebetween.

The LNMP/nucleic acid vaccine may also include active variants of a nucleic acid sequence of interest. In some instances, the variant of the nucleic acids has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a nucleic acid of interest. In some instances, the invention includes an active polypeptide encoded by a nucleic acid variant as described herein. In some instances, the active polypeptide encoded by the nucleic acid variant has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire amino acid sequence, to a sequence of a polypeptide of interest or the naturally derived polypeptide sequence.

Certain methods for expressing a nucleic acid encoding a protein may involve expression in cells, including insect, yeast, plant, bacteria, or other cells under the control of appropriate promoters. Expression vectors may include nontranscribed elements, such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green et al., Molecular Cloning: A Laboratory Manual, Fourth Edition, Cold Spring Harbor Laboratory Press, 2012.

Genetic modification using recombinant methods is generally known in the art. A nucleic acid sequence coding for a desired gene can be obtained using recombinant methods known in the art, such as, for example by screening libraries from cells expressing the gene, by deriving the gene from a vector known to include the same, or by isolating directly from cells and tissues containing the same, using standard techniques. Alternatively, a gene of interest can be produced synthetically, rather than cloned.

Expression of natural or synthetic nucleic acids is typically achieved by operably linking a nucleic acid encoding the gene of interest to a promoter, and incorporating the construct into an expression vector. Expression vectors can be suitable for replication and expression in bacteria. Expression vectors can also be suitable for replication and integration in eukaryotes. Typical cloning vectors contain transcription and translation terminators, initiation sequences, and promoters useful for expression of the desired nucleic acid sequence.

Additional promoter elements, e.g., enhancers, regulate the frequency of transcriptional initiation. Typically, these are located in the region 30-110 basepairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Alternatively, the promoter may be an inducible promoter. The use of an inducible promoter provides a molecular switch capable of turning on expression of the polynucleotide sequence to which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes may be used for identifying potentially transformed cells and for evaluating the functionality of regulatory sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., FEBS Letters 479:79-82, 2000). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some instances, an organism may be genetically modified to alter expression of one or more proteins. Expression of the one or more proteins may be modified for a specific time, e.g., development or differentiation state of the organism. In one instance, provided is a composition to alter expression of one or more proteins, e.g., proteins that affect activity, structure, or function. Expression of the one or more proteins may be restricted to a specific location(s) or widespread throughout the organism.

mRNA

The LNMP/nucleic acid vaccine may include a mRNA molecule, e.g., a mRNA molecule encoding a polypeptide. The mRNA molecule can be synthetic and modified (e.g., chemically). The mRNA molecule can be chemically synthesized or transcribed in vitro. The mRNA molecule can be disposed on a plasmid, e.g., a viral vector, bacterial vector, or eukaryotic expression vector. In some examples, the mRNA molecule can be delivered to cells by transfection, electroporation, or transduction (e.g., adenoviral or lentiviral transduction).

In some instances, the modified RNA agent of interest described herein has modified nucleosides or nucleotides. Such modifications are known and are described, e.g., in WO 2012/019168. Additional modifications are described, e.g., in WO 2015/038892; WO 2015/038892; WO 2015/089511; WO 2015/196130; WO 2015/196118 and WO 2015/196128 A2, which are herein incorporated by reference in their entirety.

In some instances, the modified RNA encoding a polypeptide of interest has one or more terminal modification, e.g., a 5′ cap structure and/or a poly-A tail (e.g., of between 100-200 nucleotides in length). The 5′ cap structure may be selected from the group consisting of CapO, Capl, ARCA, inosine, NI-methyl-guanosine, 2′fluoro-guanosine, 7-deaza-guanosine, 8-oxo-guanosine, 2-amino-guanosine, LNA-guanosine, and 2-azido-guanosine. In some cases, the modified RNAs also contain a 5′ UTR including at least one Kozak sequence, and a 3′ UTR. Such modifications are known and are described, e.g., in WO 2012/135805 and WO 2013/052523, which are incorporated herein by reference in their entirety. Additional terminal modifications are described, e.g., in WO 2014/164253 and WO 2016/011306, WO 2012/045075, and WO 2014/093924, which are incorporated herein by reference in their entirety. Chimeric enzymes for synthesizing capped RNA molecules (e.g., modified mRNA) which may include at least one chemical modification are described in WO 2014/028429, which is incorporated herein by reference in its entirety.

In some instances, a modified mRNA may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, and 3) ribozyme catalyzed. The newly formed 5′-/3′-linkage may be intramolecular or intermolecular. Such modifications are described, e.g., in WO 2013/151736.

Methods of making and purifying modified RNAs are known and disclosed in the art. For example, modified RNAs are made using only in vitro transcription (IVT) enzymatic synthesis. Methods of making IVT polynucleotides are known in the art and are described in WO 2013/151666, WO 2013/151668, WO 2013/151663, WO 2013/151669, WO 2013/151670, WO 2013/151664, WO 2013/151665, WO 2013/151671, WO 2013/151672, WO 2013/151667 and WO 2013/151736, which are incorporated herein by reference in their entirety. Methods of purification include purifying an RNA transcript including a polyA tail by contacting the sample with a surface linked to a plurality of thymidines or derivatives thereof and/or a plurality of uracils or derivatives thereof (polyT/U) under conditions such that the RNA transcript binds to the surface and eluting the purified RNA transcript from the surface (WO 2014/152031); using ion (e.g., anion) exchange chromatography that allows for separation of longer RNAs up to 10,000 nucleotides in length via a scalable method (WO 2014/144767); and subjecting a modified mRNA sample to DNAse treatment (WO 2014/152030).

Formulations of modified RNAs are known and are described, e.g., in WO 2013/090648. For example, the formulation may be, but is not limited to, nanoparticles, poly(lactic-co-glycolic acid)(PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs) and combinations thereof.

Modified RNAs encoding polypeptides in the fields of human disease, antibodies, viruses, and a variety of in vivo settings are known and are disclosed in for example, Table 6 of International Publication Nos. WO 2013/151666, WO 2013/151668, WO 2013/151663, WO 2013/151669, WO 2013/151670, WO 2013/151664, WO 2013/151665, WO 2013/151736; Tables 6 and 7 International Publication No. WO 2013/151672; Tables 6, 178 and 179 of International Publication No. WO 2013/151671; Tables 6, 185 and 186 of International Publication No WO 2013/151667; which are incorporated herein by reference in their entirety. Any of the foregoing may be synthesized as an IVT polynucleotide, chimeric polynucleotide or a circular polynucleotide, and each may include one or more modified nucleotides or terminal modifications.

### Inhibitory RNA

In some instances, the LNMP/nucleic acid vaccine includes an inhibitory RNA molecule, e.g., that acts via the RNA interference (RNAi) pathway. In some instances, the inhibitory RNA molecule decreases the level of gene expression in a target organism and/or decreases the level of a protein in the target organism. In some instances, the inhibitory RNA molecule inhibits expression of a target gene. For example, an inhibitory RNA molecule may include a short interfering RNA or its precursor, short hairpin RNA, and/or a microRNA or its precursor that targets a gene in the target organism. Certain RNA molecules can inhibit gene expression through the biological process of RNA interference (RNAi). RNAi molecules include RNA or RNA-like structures typically containing 15-50 base pairs (such as about 18-25 base pairs) and having a nucleobase sequence identical (or complementary) or nearly identical (or substantially complementary) to a coding sequence in an expressed target gene within the cell. RNAi molecules include, but are not limited to short interfering RNAs (siRNAs), double-strand RNAs (dsRNA), short hairpin RNAs (shRNA), meroduplexes, dicer substrates, and multivalent RNA interference (U.S. Pat. Nos. 8,084,599 8,349,809, 8,513,207 and 9,200,276, which are incorporated herein by reference in their entirety). The inhibitory RNA molecule can be chemically synthesized or transcribed in vitro.

Additional examples of the inhibitory RNA molecules include those described in details in International Patent Application Publication No. WO 2021/041301, which is incorporated herein by reference in its entirety.

### Circular Polyribonucleotides

The LNMP/nucleic acid vaccine includes a circular polyribonucleotide molecule, e.g., a circRNA encoding a polypeptide. The circular polyribonucleotide comprises the elements as described below as well as an expression sequence encoding the polypeptide. In some embodiments, the circular polyribonucleotide includes any feature, or any combination of features as disclosed in International Patent Publication No. WO2019/118919, which is hereby incorporated by reference in its entirety.

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

In some embodiments, the circular polyribonucleotide is between 500 nucleotides and 20,000 nucleotides, between 1,000 and 20,000 nucleotides, between 2,000 and 20,000 nucleotides, or between 5,000 and 20,000 nucleotides. In some embodiments, the circular polyribonucleotide is between 500 nucleotides and 10,000 nucleotides, between 1,000 and 10,000 nucleotides, between 2,000 and 10,000 nucleotides, or between 5,000 and 10,000 nucleotides.

### Internal Ribosome Entry Sites

In some embodiments, a circular polyribonucleotide described herein includes one or more internal ribosome entry site (IRES) elements. In some embodiments, the IRES is operably linked to one or more expression sequences (e.g., each IRES is operably linked to one or more expression sequences, where each expression sequence optionally encodes a polypeptide). In embodiments, the IRES is located between a heterologous promoter and the 5′ end of a coding sequence (e.g., a coding sequence encoding a polypeptide)

A suitable IRES element to include in a polyribonucleotide (e.g., the polyribonucleotide cargo of the polyribonucleotide) includes an RNA sequence capable of engaging a eukaryotic ribosome. In some embodiments, the IRES element is at least about 5 nt, at least about 8 nt, at least about 9 nt, at least about 10 nt, at least about 15 nt, at least about 20 nt, at least about 25 nt, at least about 30 nt, at least about 40 nt, at least about 50 nt, at least about 100 nt, at least about 200 nt, at least about 250 nt, at least about 350 nt, or at least about 500 nt.

In some embodiments, the IRES element is from the DNA of an organism including, but not limited to, a virus, a mammal, and a Drosophila. Such viral DNA may be from, but is not limited to, picornavirus complementary DNA (cDNA), with encephalomyocarditis virus (EMCV) cDNA and poliovirus cDNA. In one embodiment, Drosophila DNA from which an IRES element is from includes, but is not limited to, an Antennapedia gene from Drosophila melanogaster.

In some embodiments, the IRES sequence is an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stall intestine virus, Kashmir bee virus, Human rhinovirus 2 (HRV-2), Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picorna-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus (AEV), Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-I, Human BCL2, Human BiP, Human c-IAPI, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Salivirus, Cosavirus, Parechovirus, Drosophila hairless, S. cerevisiae TFIID, S. cerevisiae YAP1, Human c-src, Human FGF-I, Simian picomavirus, Turnip crinkle virus, Aichivirus, Crohivirus, Echovirus 11, an aptamer to eIF4G, Coxsackievirus B3 (CVB3) or Coxsackievirus A (CVB1/2). In yet another embodiment, the IRES is an IRES sequence of Coxsackievirus B3 (CVB3). In a further embodiment, the IRES is an IRES sequence of Encephalomyocarditis virus. In a further embodiment, the IRES is an IRES sequence of Theiler's encephalomyelitis virus.

The IRES sequence may have a modified sequence in comparison to the wild-type IRES sequence. In some embodiments, when the last nucleotide of the wild-type IRES is not a cytosine nucleic acid residue, the last nucleotide of the wild-type IRES sequence may be modified such that it is a cytosine residue. For example, the IRES sequence may be a CVB3 IRES sequence wherein the terminal adenosine residue is modified to cytosine residue. In some embodiments, the modified CVB3 IRES may have the nucleic acid sequence of:

(SEQ ID NO: 1)
TTAAAACAGCCTGTGGGTTGATCCCACCCACAGGCCCATTGGGCGCTAGC
ACTCTGGTATCACGGTACCTTTGTGCGCCTGTTTTATACCCCCTCCCCCA
ACTGTAACTTAGAAGTAACACACACCGATCAACAGTCAGCGTGGCACACC
AGCCACGTTTTGATCAAGCACTTCTGTTACCCCGGACTGAGTATCAATAG
ACTGCTCACGCGGTTGAAGGAGAAAGCGTTCGTTATCCGGCCAACTACTT
CGAAAAACCTAGTAACACCGTGGAAGTTGCAGAGTGTTTCGCTCAGCACT
ACCCCAGTGTAGATCAGGTCGATGAGTCACCGCATTCCCCACGGGCGACC
GTGGCGGTGGCTGCGTTGGCGGCCTGCCCATGGGGAAACCCATGGGACGC
TCTAATACAGACATGGTGCGAAGAGTCTATTGAGCTAGTTGGTAGTCCTC
CGGCCCCTGAATGCGGCTAATCCTAACTGCGGAGCACACACCCTCAAGCC
AGAGGGCAGTGTGTCGTAACGGGCAACTCTGCAGCGGAACCGACTACTTT
GGGTGTCCGTGTTTCATTTTATTCCTATACTGGCTGCTTATGGTGACAAT
TGAGAGATCGTTACCATATAGCTATTGGATTGGCCATCCGGTGACTAATA
GAGCTATTATATATCCCTTTGTTGGGTTTATACCACTTAGCTTGAAAGAG
GTTAAAACATTACAATTCATTGTTAAGTTGAATACAGCAAC

In some embodiments, the IRES sequence is an Enterovirus 71 (EV71) IRES. In some embodiments, the terminal guanosine residue of the EV71 IRES sequence is modified to a cytosine residue. In some embodiments, the modified EV71 IRES may have the nucleic acid sequence of:

(SEQ ID NO: 2)
TTAAAACAGCTGTGGGTTGTCACCCACCCACAGGGTCCACTGGGCGCTAG
TACACTGGTATCTCGGTACCTTTGTACGCCTGTTTTATACCCCCTCCCTG
ATTTGCAACTTAGAAGCAACGCAAACCAGATCAATAGTAGGTGTGACATA
CCAGTCGCATCTTGATCAAGCACTTCTGTATCCCCGGACCGAGTATCAAT
AGACTGTGCACACGGTTGAAGGAGAAAACGTCCGTTACCCGGCTAACTAC
TTCGAGAAGCCTAGTAACGCCATTGAAGTTGCAGAGTGTTTCGCTCAGCA
CTCCCCCCGTGTAGATCAGGTCGATGAGTCACCGCATTCCCCACGGGCGA
CCGTGGCGGTGGCTGCGTTGGCGGCCTGCCTATGGGGTAACCCATAGGAC
GCTCTAATACGGACATGGCGTGAAGAGTCTATTGAGCTAGTTAGTAGTCC
TCCGGCCCCTGAATGCGGCTAATCCTAACTGCGGAGCACATACCCTTAAT
CCAAAGGGCAGTGTGTCGTAACGGGCAACTCTGCAGCGGAACCGACTACT
TTGGGTGTCCGTGTTTCTTTTTATTCTTGTATTGGCTGCTTATGGTGACA
ATTAAAGAATTGTTACCATATAGCTATTGGATTGGCCATCCAGTGTCAAA
CAGAGCTATTGTATATCTCTTTGTTGGATTCACACCTCTCACTCTTGAAA
CGTTACACACCCTCAATTACATTATACTGCTGAACACGAAGCGGCCACC

In some embodiments, the polyribonucleotide (e.g., the polyribonucleotide cargo of the polyribonucleotide) includes at least one IRES flanking at least one (e.g., 2, 3, 4, 5 or more) expression sequence. In some embodiments, the IRES flanks both sides of at least one (e.g., 2, 3, 4, 5 or more) expression sequence. In some embodiments, the polyribonucleotide (e.g., the polyribonucleotide cargo of the polyribonucleotide) includes one or more IRES sequences on one or both sides of each expression sequence, leading to separation of the resulting peptide(s) and or polypeptide(s). For example, a polyribonucleotide (e.g., the polyribonucleotide cargo of the polyribonucleotide) described herein may include a first IRES operably linked to a first expression sequence (e.g., encoding a first polypeptide) and a second IRES operably linked to a second expression sequence (e.g., encoding a second polypeptide).

In some embodiments, a polyribonucleotide (e.g., the polyribonucleotide cargo of the polyribonucleotide) described herein includes an IRES (e.g., an IRES operably linked to a coding region). For example, the polyribonucleotide (e.g., the polyribonucleotide cargo of the polyribonucleotide) may include any IRES as described in Chen et al. Mol. Cell 81(20):4300-4318, 2021; Jopling et al. Oncogene 20:2664-2670, 2001; Baranick et al. PNAS 105(12):4733-4738, 2008; Lang et al. Molecular Biology of the Cell 13(5):1792-1801, 2002; Dorokhov et al. PNAS 99(8):5301-5306, 2002; Wang et al. Nucleic Acids Research 33(7):2248-2258, 2005; Petz et al. Nucleic Acids Research 35(8):2473-2482, 2007, Chen et al. SCIENCE 268:415-417, 1995; Fan et al. NATURE COMMUNICATION 13(1):3751-3765, 2022, and International Publication No. WO2021/263124 each of which is hereby incorporated by reference in their entirety.

### Regulatory Elements

In some embodiments, the circular polyribonucleotide includes a regulatory element, e.g., a sequence that modifies expression of an expression sequence within the circular polyribonucleotide. In some embodiments, the polyribonucleotide described herein (e.g., the polyribonucleotide cargo of the polyribonucleotide) includes one or more regulatory elements.

A regulatory element may include a sequence that is located adjacent to an expression sequence that encodes an expression product (e.g., polypeptide). A regulatory element may be operatively linked to the adjacent sequence. A regulatory element may increase an amount of product expressed as compared to an amount of the expressed product when no regulatory element exists. A regulatory element may be used to increase the expression of one or more polypeptide(s) encoded by a polyribonucleotide (e.g., the polyribonucleotide cargo of the polyribonucleotide). Likewise, a regulatory element may be used to decrease the expression of one or more polypeptide(s) encoded by a polyribonucleotide (e.g., the polyribonucleotide cargo of the polyribonucleotide). In some embodiments, a regulatory element may be used to increase expression of a polypeptide and another regulatory element may be used to decrease expression of another polypeptide on the same polyribonucleotide (e.g., the polyribonucleotide cargo of the polyribonucleotide). In addition, one regulatory element can increase amount(s) of product(s) (e.g., polypeptide(s)) expressed for multiple expression sequences attached in tandem. Hence, one regulatory element can enhance the expression of one or more expression sequences. Multiple regulatory elements can also be used, for example, to differentially regulate expression of different expression sequences.

In some embodiments, the regulatory element is a translation modulator. A translation modulator can modulate translation of the expression sequence in the circular polyribonucleotide. A translation modulator can be a translation enhancer or suppressor. In some embodiments, the circular polyribonucleotide includes at least one translation modulator adjacent to at least one expression sequence. In some embodiments, the circular polyribonucleotide includes a translation modulator adjacent each expression sequence. In some embodiments, the translation modulator is present on one or both sides of each expression sequence, leading to separation of the expression products, e.g., polypeptide(s).

In some embodiments, the regulatory element is a microRNA (miRNA) or a miRNA binding site.

Further examples of regulatory elements are described, e.g., in paragraphs [0154]-[0161] of International Patent Publication No. WO2019/118919, which is hereby incorporated by reference in its entirety.

### Signal Sequences

In some embodiments, a polypeptide expressed from a circular or linear polyribonucleotide disclosed herein includes a secreted protein, for example, a protein that naturally includes a signal sequence, or one that does not usually encode a signal sequence but is modified to contain one. In some embodiments, the polypeptide encoded by the circular or linear polyribonucleotide includes a secretion signal. For example, the secretion signal may be the naturally encoded secretion signal for a secreted protein. In another example, the secretion signal may be a modified secretion signal for a secreted protein. In other embodiments, the polypeptide encoded by the circular or linear polyribonucleotide does not include a secretion signal.

In some embodiments, a polyribonucleotide (e.g., circular polyribonucleotide, linear polyribonucleotide, polyribonucleotide cargo of the polyribonucleotide) encodes multiple copies of the same polypeptide (e.g., one, two, three, four, five, six, seven, eight, nine, ten, or more). In some embodiments, at least one copy of the polypeptide includes a signal sequence and at least one copy of the polypeptide does not include a signal sequence. In some embodiments, a polyribonucleotide (e.g., circular polyribonucleotide, linear polyribonucleotide, polyribonucleotide cargo of the polyribonucleotide) encodes plurality of polypeptides (e.g., a plurality of different polypeptides or a plurality of polypeptides having less than 100% sequence identity), where at least one of the plurality of polypeptides includes a signal sequence and at least one copy of the plurality of polypeptides does not include a signal sequence.

In some embodiments, the signal sequence is a wild-type signal sequence that is present on the N-terminus of the corresponding wild-type polypeptide, e.g., when expressed endogenously. In some embodiments, the signal sequence is heterologous to the polypeptide, e.g., is not present when the wild-type polypeptide is expressed endogenously. A polyribonucleotide sequence encoding a polypeptide may be modified to remove the nucleotide sequence encoding a wild-type signal sequence and/or add a sequence encoding a heterologous signal sequence.

A polypeptide encoded by a polyribonucleotide may include a signal sequence that directs the polypeptide to the secretory pathway. In some embodiments, the signal sequence may direct the polypeptide to reside in certain organelles (e.g., the endoplasmic reticulum, Golgi apparatus, or endosomes). In some embodiments, the signal sequence directs the polypeptide to be secreted from the cell. For secreted proteins, the signal sequence may be cleaved after secretion, resulting in a mature protein. In other embodiments, the signal sequence may become embedded in the membrane of the cell or certain organelles, creating a transmembrane segment that anchors the protein to the membrane of the cell, endoplasmic reticulum, or Golgi apparatus. In certain embodiments, the signal sequence of a transmembrane protein is a short sequence at the N-terminal of the polypeptide. In other embodiments, the first transmembrane domain acts as the first signal sequence, which targets the protein to the membrane. In some embodiments, a polypeptide encoded by a polyribonucleotide includes either a secretion signal sequence, a transmembrane insertion signal sequence, or does not include a signal sequence.

### Cleavage Domains

A circular polyribonucleotide of the disclosure can include a cleavage domain (e.g., a stagger element or a cleavage sequence).

The term “stagger element” refers to a moiety, such as a nucleotide sequence, that induces ribosomal pausing during translation. In some embodiments, the stagger element is a non-conserved sequence of amino-acids with a strong alpha-helical propensity followed by the consensus sequence -D(V/I)ExNPGP, where x=any amino acid (SEQ ID NO: 3). In some embodiments, the stagger element may include a chemical moiety, such as glycerol, a non-nucleic acid linking moiety, a chemical modification, a modified nucleic acid, or any combination thereof.

In some embodiments, the circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, e.g., peptide(s) and or polypeptide(s). In some embodiments, the stagger element is a portion of the one or more expression sequences. In some embodiments, the circular polyribonucleotide includes one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element includes a portion of an expression sequence of the one or more expression sequences.

In some embodiments, the circular polyribonucleotide includes a stagger element. To avoid production of a continuous expression product, e.g., peptide or polypeptide, while maintaining rolling circle translation, a stagger element may be included to induce ribosomal pausing during translation. In some embodiments, the stagger element is at 3′ end of at least one of the one or more expression sequences. The stagger element can be configured to stall a ribosome during rolling circle translation of the circular polyribonucleotide. The stagger element may include, but is not limited to a 2A-like, or CHYSEL (SEQ ID NO: 4) (cis-acting hydrolase element) sequence. In some embodiments, the stagger element encodes a sequence with a C-terminal consensus sequence that is X1X2X3EX5NPGP (SEQ ID NO: 5), where X1 is absent or G or H, X2 is absent or D or G, X3 is D or V or I or S or M, and X5 is any amino acid. In some embodiments, this sequence includes a non-conserved sequence of amino-acids with a strong alpha-helical propensity followed by the consensus sequence -D(V/I)EXNPGP (SEQ ID NO: 6), where x=any amino acid. Some nonlimiting examples of stagger elements includes GDVESNPGP (SEQ ID NO: 7), GDIEENPGP (SEQ ID NO: 8), VEPNPGP (SEQ ID NO: 9), IETNPGP (SEQ ID NO: 10), GDIESNPGP (SEQ ID NO: 11), GDVELNPGP (SEQ ID NO: 12), GDIETNPGP (SEQ ID NO: 13), GDVENPGP (SEQ ID NO: 14), GDVEENPGP (SEQ ID NO: 15), GDVEQNPGP (SEQ ID NO: 16), IESNPGP (SEQ ID NO: 17), GDIELNPGP (SEQ ID NO: 18), HDIETNPGP (SEQ ID NO: 19), HDVETNPGP (SEQ ID NO: 20), HDVEMNPGP (SEQ ID NO: 21), GDMESNPGP (SEQ ID NO: 22), GDVETNPGP (SEQ ID NO: 23), GDIEQNPGP (SEQ ID NO: 24), and DSEFNPGP (SEQ ID NO: 25).

In some embodiments, the stagger element described herein cleaves an expression product, such as between G and P of the consensus sequence described herein. As one non-limiting example, the circular polyribonucleotide includes at least one stagger element to cleave the expression product. In some embodiments, the circular polyribonucleotide includes a stagger element adjacent to at least one expression sequence. In some embodiments, the circular polyribonucleotide includes a stagger element after each expression sequence. In some embodiments, the circular polyribonucleotide includes a stagger element is present on one or both sides of each expression sequence, leading to translation of individual peptide(s) and or polypeptide(s) from each expression sequence.

In some embodiments, a stagger element includes one or more modified nucleotides or unnatural nucleotides that induce ribosomal pausing during translation. Unnatural nucleotides may include peptide nucleic acid (PNA), Morpholino and locked nucleic acid (LNA), as well as glycol nucleic acid (GNA) and threose nucleic acid (TNA). Examples such as these are distinguished from naturally occurring DNA or RNA by changes to the backbone of the molecule. Exemplary modifications can include any modification to the sugar, the nucleobase, the internucleoside linkage (e.g., to a linking phosphate/to a phosphodiester linkage/to the phosphodiester backbone), and any combination thereof that can induce ribosomal pausing during translation. Some of the exemplary modifications provided herein are described elsewhere herein.

In some embodiments, the stagger element is present in the circular polyribonucleotide in other forms. For example, in some exemplary circular polyribonucleotides, a stagger element includes a termination element of a first expression sequence in the circular polyribonucleotide, and a nucleotide spacer sequence that separates the termination element from a first translation initiation sequence of an expression succeeding the first expression sequence. In some examples, the first stagger element of the first expression sequence is upstream of (5′ to) a first translation initiation sequence of the expression succeeding the first expression sequence in the circular polyribonucleotide. In some cases, the first expression sequence and the expression sequence succeeding the first expression sequence are two separate expression sequences in the circular polyribonucleotide. The distance between the first stagger element and the first translation initiation sequence can enable continuous translation of the first expression sequence and its succeeding expression sequence.

In some embodiments, the first stagger element includes a termination element and separates an expression product of the first expression sequence from an expression product of its succeeding expression sequences, thereby creating discrete expression products. In some cases, the circular polyribonucleotide including the first stagger element upstream of the first translation initiation sequence of the succeeding sequence in the circular polyribonucleotide is continuously translated, while a corresponding circular polyribonucleotide including a stagger element of a second expression sequence that is upstream of a second translation initiation sequence of an expression sequence succeeding the second expression sequence is not continuously translated. In some cases, there is only one expression sequence in the circular polyribonucleotide, and the first expression sequence and its succeeding expression sequence are the same expression sequence. In some exemplary circular polyribonucleotides, a stagger element includes a first termination element of a first expression sequence in the circular polyribonucleotide, and a nucleotide spacer sequence that separates the termination element from a downstream translation initiation sequence. In some such examples, the first stagger element is upstream of (5′ to) a first translation initiation sequence of the first expression sequence in the circular polyribonucleotide. In some cases, the distance between the first stagger element and the first translation initiation sequence enables continuous translation of the first expression sequence and any succeeding expression sequences.

In some embodiments, the first stagger element separates one round expression product of the first expression sequence from the next round expression product of the first expression sequences, thereby creating discrete expression products. In some cases, the circular polyribonucleotide including the first stagger element upstream of the first translation initiation sequence of the first expression sequence in the circular polyribonucleotide is continuously translated, while a corresponding circular polyribonucleotide including a stagger element upstream of a second translation initiation sequence of a second expression sequence in the corresponding circular polyribonucleotide is not continuously translated. In some cases, the distance between the second stagger element and the second translation initiation sequence is at least 2×, 3×, 4×, 5×, 6×, 7×, 8×, 9×, or 10× greater in the corresponding circular polyribonucleotide than a distance between the first stagger element and the first translation initiation in the circular polyribonucleotide. In some cases, the distance between the first stagger element and the first translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater. In some embodiments, the distance between the second stagger element and the second translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater than the distance between the first stagger element and the first translation initiation. In some embodiments, the circular polyribonucleotide includes more than one expression sequence.

Examples of stagger elements are described in paragraphs [0172]-[0175] of International Patent Publication No. WO2019/118919, which is hereby incorporated by reference in its entirety.

In some embodiments, a plurality of polypeptides encoded by a circular ribonucleotide may be separated by an IRES between each polypeptide (e.g., each polypeptide is operably linked to a separate IRES). For example, a circular polyribonucleotide may include a first IRES operably linked to a first expression sequence and a second IRES operably linked to a second expression sequence. The IRES may be the same IRES between all polypeptides. The IRES may be different between different polypeptides.

In some embodiments, the plurality of polypeptides may be separated by a 2A self-cleaving peptide. For example, a circular polyribonucleotide may encode an IRES operably linked to an open reading frame encoding a first polypeptide, a 2A, and a second polypeptide.

In some embodiments, the plurality of polypeptides may be separated by a protease cleavage site (e.g., a furin cleavage site). For example, a circular polyribonucleotide may encode an IRES operably linked to an open reading frame encoding a first polypeptide, a protease cleavage site (e.g., a furin cleavage site), and a second polypeptide.

In some embodiments, the plurality of polypeptides may be separated by a 2A self-cleaving peptide and a protease cleavage site (e.g., a furin cleavage site). For example, a circular polyribonucleotide may encode an IRES operably linked to an open reading frame encoding a first polypeptide, a 2A, a protease cleavage site (e.g., a furin cleavage site), and a second polypeptide. A circular polyribonucleotide may also encode an IRES operably linked to an open reading frame encoding a first polypeptide, a protease cleavage site (e.g., a furin cleavage site), a 2A, and a second polypeptide. A tandem 2A and furin cleavage site may be referred to as a furin-2A (which includes furin-2A or 2A-furin, arranged in either orientation).

Furthermore, the plurality of polypeptides encoded by the circular ribonucleotide may be separated by both IRES and 2A sequences. For example, an IRES may be between one polypeptide and a second polypeptide while a 2A peptide may be between the second polypeptide and the third polypeptide. The selection of a particular IRES or 2A self-cleaving peptide may be used to control the expression level of a polypeptide under control of the IRES or 2A sequence. For example, depending on the IRES and or 2A peptide selected, expression on the polypeptide may be higher or lower.

In some embodiments, a circular polyribonucleotide includes at least one cleavage sequence. In some embodiments, the cleavage sequence is adjacent to an expression sequence. In some embodiments, the cleavage sequence is between two expression sequences. In some embodiments, cleavage sequence is included in an expression sequence. In some embodiments, the circular polyribonucleotide includes from 2 to 10 cleavage sequences. In some embodiments, the circular polyribonucleotide includes from 2 to 5 cleavage sequences. In some embodiments, the multiple cleavage sequences are between multiple expression sequences; for example, a circular polyribonucleotide may include three expression sequences two cleavage sequences such that there is a cleavage sequence in between each expression sequence. In some embodiments, the circular polyribonucleotide includes a cleavage sequence, such as in an immolating circRNA or cleavable circRNA or self-cleaving circRNA. In some embodiments, the circular polyribonucleotide includes two or more cleavage sequences, leading to separation of the circular polyribonucleotide into multiple products, e.g., miRNAs, linear RNAs, smaller circular polyribonucleotide, etc.

In some embodiments, a cleavage sequence includes a ribozyme RNA sequence. A ribozyme (from ribonucleic acid enzyme, also called RNA enzyme or catalytic RNA) is an RNA molecule that catalyzes a chemical reaction. Many natural ribozymes catalyze either the hydrolysis of one of their own phosphodiester bonds, or the hydrolysis of bonds in other RNA, but they have also been found to catalyze the aminotransferase activity of the ribosome. Catalytic RNA can be “evolved” by in vitro methods. Similar to riboswitch activity discussed above, ribozymes and their reaction products can regulate gene expression. In some embodiments, a catalytic RNA or ribozyme can be placed within a larger non-coding RNA such that the ribozyme is present at many copies within the cell for the purposes of chemical transformation of a molecule from a bulk volume. In some embodiments, aptamers and ribozymes can both be encoded in the same non-coding RNA.

In some embodiments, the cleavage sequence encodes a cleavable polypeptide linker. For example, a circular polyribonucleotide may encode two or more polypeptides, e.g., where the two or more polypeptides are encoded by a single open-reading frame (ORF). For example, two or more polypeptides may be encoded by a single open-reading frame, the expression of which is controlled by an IRES. In some embodiments, the ORF further encodes a polypeptide linker, e.g., such that the expression product of the ORF encodes two or more polypeptides each separated by a sequence encoding a polypeptide linker (e.g., a linker of 5-200, 5 to 100, 5 to 50, 5 to 20, 50 to 100, or 50 to 200 amino acids). The polypeptide linker may include a cleavage site, for example, a cleavage site recognized and cleaved by a protease (e.g., an endogenous protease in a subject following administration of the circular polyribonucleotide to that subject). In such embodiments, a single expression product including the amino acid sequence of two or more polypeptides is cleaved upon expression, such that the two or more polypeptides are separated following expression. Exemplary protease cleavage sites are known to those of skill in the art, for example, amino acid sequences that act as protease cleavage sites recognized by a metalloproteinase (e.g., a matrix metalloproteinase (MMP), such as any one or more of MMPs 1-28), a disintegrin and metalloproteinase (ADAM, such as any one or more of ADAMs 2, 7-12, 15, 17-23, 28-30 and 33), a serine protease (e.g., furin), urokinase-type plasminogen activator, matriptase, a cysteine protease, an aspartic protease, or a cathepsin protease. In some embodiments, the protease is MMP9 or MMP2. In some embodiments, the protease is matriptase.

In some embodiments, a circular polyribonucleotide described herein is an immolating circular polyribonucleotide, a cleavable circular polyribonucleotide, or a self-cleaving circular polyribonucleotide. A circular polyribonucleotide can deliver cellular components including, for example, RNA, long non-coding RNA (lncRNA), long intergenic non-coding RNA (lincRNA), microRNA (miRNA), transfer RNA (tRNA), ribosomal RNA (rRNA), small nucleolar RNA (snoRNA), non-coding RNA (ncRNA), small interfering RNA (siRNA), or small hairpin RNA (shRNA). In some embodiments, a circular polyribonucleotide includes miRNA separated by (i) self-cleavable elements; (ii) cleavage recruitment sites; (iii) degradable linkers; (iv) chemical linkers; and/or (v) spacer sequences. In some embodiments, circRNA includes siRNA separated by (i) self-cleavable elements; (ii) cleavage recruitment sites (e.g., ADAR); (iii) degradable linkers (e.g., glycerol); (iv) chemical linkers; and/or (v) spacer sequences. Non-limiting examples of self-cleavable elements include hammerhead, splicing element, hairpin, hepatitis delta virus (HDV), Varkud Satellite (VS), and glmS ribozymes.

### Translation Initiation Sequences

In some embodiments, the circular polyribonucleotide described herein includes at least one translation initiation sequence. In some embodiments, the polyribonucleotide (e.g., the polyribonucleotide cargo of the polyribonucleotide) includes a translation initiation sequence operably linked to an expression sequence.

In some embodiments, the polyribonucleotide encodes a polypeptide and may include a translation initiation sequence, e.g., a start codon. In some embodiments, the translation initiation sequence includes a Kozak or Shine-Dalgarno sequence. In some embodiments, the polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the polyribonucleotide includes at least one translation initiation sequence adjacent to an expression sequence. In some embodiments, the translation initiation sequence provides conformational flexibility to the polyribonucleotide. In some embodiments, the translation initiation sequence is within a substantially single stranded region of the polyribonucleotide. Further examples of translation initiation sequences are described in paragraphs [0163]-[0165] of International Patent Publication No. WO2019/118919, which is hereby incorporated by reference in its entirety.

The polyribonucleotide may include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation may initiate on the first start codon or may initiate downstream of the first start codon.

In some embodiments, the polyribonucleotide may initiate at a codon which is not the first start codon, e.g., AUG. Translation of the polyribonucleotide may initiate at an alternative translation initiation sequence, such as, but not limited to, ACG, AGG, AAG, CTG/CUG, GTG/GUG, ATA/AUA, ATT/AUU, TTG/UUG. In some embodiments, translation begins at an alternative translation initiation sequence under selective conditions, e.g., stress induced conditions. As a non-limiting example, the translation of the polyribonucleotide may begin at alternative translation initiation sequence, such as ACG. As another non-limiting example, the polyribonucleotide translation may begin at alternative translation initiation sequence, CTG/CUG. As another non-limiting example, the polyribonucleotide translation may begin at alternative translation initiation sequence, GTG/GUG. As another non-limiting example, the polyribonucleotide may begin translation at a repeat-associated non-AUG (RAN) sequence, such as an alternative translation initiation sequence that includes short stretches of repetitive RNA e.g., CGG, GGGGCC (SEQ DI NO: 93), CAG, CTG.

### Termination Elements

In some embodiments, the polyribonucleotide described herein (e.g., the polyribonucleotide cargo of the polyribonucleotide) includes least one termination element. In some embodiments, the polyribonucleotide includes a termination element operably linked to an expression sequence. In some embodiments, the polynucleotide lacks a termination element.

In some embodiments, the polyribonucleotide includes one or more expression sequences, and each expression sequence may or may not have a termination element. In some embodiments, the polyribonucleotide includes one or more expression sequences, and the expression sequences lack a termination element, such that the polyribonucleotide is continuously translated. Exclusion of a termination element may result in rolling circle translation or continuous expression of expression product.

In some embodiments, the circular polyribonucleotide includes one or more expression sequences, and each expression sequence may or may not have a termination element. In some embodiments, the circular polyribonucleotide includes one or more expression sequences, and the expression sequences lack a termination element, such that the circular polyribonucleotide is continuously translated. Exclusion of a termination element may result in rolling circle translation or continuous expression of expression product, e.g., peptides or polypeptides, due to lack of ribosome stalling or fall-off. In such an embodiment, rolling circle translation expresses a continuous expression product through each expression sequence. In some other embodiments, a termination element of an expression sequence can be part of a stagger element. In some embodiments, one or more expression sequences in the circular polyribonucleotide includes a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the circular polyribonucleotide.

In some embodiments, the circular polyribonucleotide includes a termination element at the end of one or more expression sequences. In some embodiments, one or more expression sequences includes two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Generally, termination elements include an in-frame nucleotide triplet that signals termination of translation, e.g., UAA, UGA, UAG. In some embodiments, one or more termination elements in the circular polyribonucleotide are frame-shifted termination elements, such as but not limited to, off-frame or −1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell. In some embodiments, the termination element is a stop codon.

Further examples of termination elements are described in paragraphs [0169]-[0170] of International Patent Publication No. WO2019/118919, which is hereby incorporated by reference in its entirety.

### Spacer Sequences

In some embodiments, the polyribonucleotide (e.g., circular polyribonucleotide, linear polyribonucleotide) described herein includes one or more spacer sequences. A spacer or spacer sequence refers to any contiguous nucleotide sequence (e.g., of one or more nucleotides) that provides distance or flexibility between two adjacent polynucleotide regions. Spacers may be present in between any of the nucleic acid elements described herein. Spacer may also be present within a nucleic acid element as described herein.

For example, wherein a nucleic acid includes any two or more of the following elements: (A) a 3′ catalytic intron fragment; (B) a 3′ splice site; (C) a 3′ exon fragment; (D) a polyribonucleotide cargo; (E) a 5′ exon fragment; (F) a 5′ splice site; and (G) a 5′ catalytic intron fragment; a spacer region may be present between any one or more of the elements. Any of elements (A), (B), (C), (D), (E), (F), or (G) may be separated by a spacer sequence, as described herein. For example, there may be a spacer between (A) and (B), between (B) and (C), between (C) and (D), between (D) and (E), between (E) and (F), or between (F) and (G).

In some embodiments, the polyribonucleotide (e.g., circular polyribonucleotide, linear polyribonucleotide) further includes a first spacer sequence between the 5′ exon fragment of (C) and the polyribonucleotide cargo of (D). The spacer may be, e.g., at least 5 (e.g., at least 10, at least 15, at least 20) ribonucleotides in length. In some embodiments, the polyribonucleotide further includes a second spacer sequence between the polyribonucleotide cargo of (D) and the 5′ exon fragment of (E).

A spacer sequence may be used to separate an IRES from adjacent structural elements to maintain the structure and function of the IRES or the adjacent element. A spacer can be specifically engineered depending on the IRES. In some embodiments, an RNA folding computer software, such as RNAFold, can be utilized to guide designs of the various elements of the vector, including the spacers. Thus, in one embodiment, the spacer sequence is between the IRES and the 3′ exon fragment or the 5′ exon fragment. In other embodiments, the spacer sequence is between the expression sequence and the 3′ exon fragment. In other embodiments, the spacer sequence is adjacent to the 5′ exon fragment or the 3′ exon fragment.

The spacer may be, e.g., at least 3 (e.g., at least 10, at least 15, at least 20) ribonucleotides in length. In some embodiments, each spacer sequence is at least 3 (e.g., at least 10, at least 15, at least 20) ribonucleotides in length. Each spacer region may be, e.g., from 5 to 800 (e.g., 10, 20, 30, 40, 50, 60, 70, 80, 90, 100, 150, 200, 250, 300, 350, 400, 450, 500, 550, 600, 650, 700, 750 or 800) ribonucleotides in length. In some embodiments, the spacer sequence is at least about 60 ribonucleotides in length. In some embodiments, the spacer sequence is from about 60 to about 650 ribonucleotides in length.

In some embodiments, the first spacer sequence, the second spacer sequence, or the first spacer sequence and the second spacer sequence may include a poly(X) sequence. In some embodiments the first spacer sequence and the second spacer sequence, or the first spacer sequence and the second spacer sequence, may include a poly(A) sequence. The first spacer sequence, the second spacer sequence, or the first spacer sequence and the second spacer sequence, may include a poly(A-C) sequence. In some embodiments, the first spacer sequence, the second spacer sequence, or the first spacer sequence and the second spacer sequence includes a poly(A-G) sequence. In some embodiments, the first spacer sequence, the second spacer sequence, or the first spacer sequence and the second spacer sequence includes a poly(A-T) sequence. In some embodiments, the first spacer sequence, the second spacer sequence, or the first spacer sequence and the second spacer sequence includes a random sequence.

Spacers may also be present within a nucleic acid region described herein. For example, a polynucleotide cargo region may include one or multiple spacers. Spacers may separate regions within the polynucleotide cargo.

In some embodiments, the spacer sequence can be, for example, at least 10 nucleotides in length, at least 15 nucleotides in length, or at least 70 nucleotides in length. In some embodiments, the spacer sequence is at least 4, 5, 6, 7, 8, 9, 10, 11, 12, 13, 14, 15, 16, 17, 18, 19, 20, 25, 30, 35, 40, 45, 50, 55, 60, 65, or 70 nucleotides in length. In some embodiments, the spacer sequence is no more than 800, 700, 600, 500, 400, 300, 300, 100, 90, 80, 70, 60, 50, 45, 40, 35 or 30 nucleotides in length. In some embodiments the spacer sequence is from 20 to 70 nucleotides in length. In certain embodiments, the spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides in length.

The spacer sequences may be poly(X) sequences, poly(A) sequences, poly(A-T) sequences, poly(A-U) sequences, poly(A-C) sequences, poly(A-G) sequences, poly(G), poly(C) sequences, poly(U) sequences, or random sequences.

Exemplary spacer sequences are described in paragraphs [0293]-[0302] of International Patent Publication No. WO2019/118919, which is hereby incorporated by reference in its entirety.

In some embodiments, the circular polyribonucleotide includes a 5′ spacer sequence (e.g., between the 5′ annealing region and the polyribonucleotide cargo). In some embodiments, the 5′ spacer sequence is at least about 60 nucleotides in length. In another embodiment, the 5′ spacer sequence is at least 100 nucleotides in length. In a further embodiment, the 5′ spacer sequence is at least about 200 nucleotides in length. In other embodiment, the 5′ spacer sequence is at least 300 nucleotides in length. In another embodiment, the 5′ spacer sequence is at least about 400 nucleotides in length. In another embodiment, the 5′ spacer sequence is at least about 500 nucleotides in length. In other embodiment, the 5′ spacer sequence is at least about 600 nucleotides in length. In other embodiment, the 5′ spacer sequence is at least 700 nucleotides in length. In one embodiment, the 5′ space is a poly(X) sequence. In one embodiment, the 5′ spacer sequence is a poly(A) sequence. In another embodiment, the 5′ spacer sequence is a poly(A-C) sequence. In some embodiments, the 5′ spacer sequence includes a poly(A-G) sequence. In some embodiments, the 5′ spacer sequence includes a poly(A-U) sequence. In some embodiments, the 5′ spacer sequence includes a random sequence.

In some embodiments, the polyribonucleotide (e.g., circular polyribonucleotide, linear polyribonucleotide) includes a 3′ spacer sequence (e.g., between the 3′ annealing region and the polyribonucleotide cargo). In some embodiments, the polyribonucleotide includes a 3′ spacer sequence (e.g., between the 3′ annealing region and the polyribonucleotide cargo). In some embodiments, the 3′ spacer sequence is at least about 60 nucleotides in length. In another embodiment, the 3′ spacer sequence is at least 100 nucleotides in length. In a further embodiment, the 3′ spacer sequence is at least about 200 nucleotides in length. In other embodiment, the 3′ spacer sequence is at least 300 nucleotides in length. In another embodiment, the 3′ spacer sequence is at least about 400 nucleotides in length. In another embodiment, the 3′ spacer sequence is at least about 500 nucleotides in length. In other embodiment, the 3′ spacer sequence is at least about 600 nucleotides in length. In other embodiment, the 3′ spacer sequence is at least 700 nucleotides in length. In one embodiment, the 3′ spacer sequence is a poly(X) sequence. In one embodiment, the 3′ spacer sequence is a poly(A) sequence. In another embodiment, the 3′ spacer sequence is a poly(A-C) sequence. In some embodiments, the 3′ spacer sequence includes a poly(A-G) sequence. In some embodiments, the 3′ spacer sequence includes a poly(A-U) sequence. In some embodiments, the 3′ spacer sequence includes a random sequence.

In one embodiment, the polyribonucleotide (e.g., circular polyribonucleotide, linear polyribonucleotide) includes a 5′ spacer sequence, but not a 3′ spacer sequence. In another embodiment, the circular polyribonucleotide includes a 3′ spacer sequence, but not a 5′ spacer sequence. In another embodiment, the polyribonucleotide includes neither a 5′ spacer sequence, nor a 3′ spacer sequence. In another embodiment, the polyribonucleotide does not include an IRES sequence. In a further embodiment, the polyribonucleotide does not include an IRES sequence, a 5′ spacer sequence or a 3′ spacer sequence.

In some embodiments, the spacer sequence includes at least 3 ribonucleotides, at least 4 ribonucleotides, at least 5 ribonucleotides, at least about 8 ribonucleotides, at least about 10 ribonucleotides, at least about 12 ribonucleotides, at least about 15 ribonucleotides, at least about 20 ribonucleotides, at least about 25 ribonucleotides, at least about 30 ribonucleotides, at least about 40 ribonucleotides, at least about 50 ribonucleotides, at least about 60 ribonucleotides, at least about 70 ribonucleotides, at least about 80 ribonucleotides, at least about 90 ribonucleotides, at least about 100 ribonucleotides, at least about 120 ribonucleotides, at least about 150 ribonucleotides, at least about 200 ribonucleotides, at least about 250 ribonucleotides, at least about 300 ribonucleotides, at least about 400 ribonucleotides, at least about 500 ribonucleotides, at least about 600 ribonucleotides, at least about 700 ribonucleotides, at least about 800 ribonucleotides, at least about 900 ribonucleotides, or at least about 1000 ribonucleotides.

In some embodiments, a circular polyribonucleotide includes a spacer sequence. In some embodiments, a linear polyribonucleotide includes a spacer sequence. In some embodiments, a spacer sequence may be included upstream of the translation initiation sequence of an expression sequence. In some embodiments, a spacer sequence may be included downstream of an expression sequence. In some instances, one spacer sequence for a first expression sequence is the same as or continuous with or overlapping with another spacer sequence for a second expression sequence. In some embodiments, the intron is a human intron. In some embodiments, the intron is a full-length human intron, e.g., ZKSCAN1.

Exemplary spacer sequences are described in paragraphs [0197]-[201] of International Patent Publication No. WO2019/118919, which is hereby incorporated by reference in its entirety.

In some embodiments, a circular polyribonucleotide includes a poly(A) sequence. In some embodiments, a linear polyribonucleotide includes a poly(A) sequence. Exemplary poly(A) sequences are described in paragraphs [0202]-[0205] of International Patent Publication No. WO2019/118919, which is hereby incorporated by reference in its entirety. In some embodiments, a circular polyribonucleotide lacks a poly(A) sequence. In some embodiments, a linear polyribonucleotide lacks a poly(A) sequence.

In some embodiments, a polyribonucleotide (e.g., circular polyribonucleotide, linear polyribonucleotide) includes a spacer sequence with one or more stretches of Adenosines and Uridines embedded within. These AU rich signatures may increase turnover rates of the expression product.

Introduction, removal, or modification of the spacer sequence AU rich elements (AREs) may be useful to modulate the stability, or immunogenicity (e.g., the level of one or more marker of an immune or inflammatory response) of a circular polyribonucleotide. When engineering specific polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

It should be understood that any spacer sequence from any gene may be incorporated into the respective flanking regions of a circular polyribonucleotide.

In some embodiments, a circular polyribonucleotide lacks a 5′ spacer sequence and is competent for protein expression from its one or more expression sequences. In some embodiments, the circular polyribonucleotide lacks a 3′ spacer sequence and is competent for protein expression from its one or more expression sequences. In some embodiments, the circular polyribonucleotide lacks a poly(A) sequence and is competent for protein expression from its one or more expression sequences. In some embodiments, the circular polyribonucleotide lacks a termination element and is competent for protein expression from its one or more expression sequences. In some embodiments, the circular polyribonucleotide lacks an internal ribosomal entry site and is competent for protein expression from its one or more expression sequences. In some embodiments, the circular polyribonucleotide lacks a cap and is competent for protein expression from its one or more expression sequences. In some embodiments, the circular polyribonucleotide lacks a 5′ spacer sequence, a 3′ spacer sequence, and an IRES, and is competent for protein expression from its one or more expression sequences. In some embodiments, the circular polyribonucleotide includes one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

In some embodiments, a circular polyribonucleotide lacks a 5′ spacer sequence. In some embodiments, the circular polyribonucleotide lacks a spacer sequence. In some embodiments, the circular polyribonucleotide lacks a poly(X) sequence. In some embodiments, the circular polyribonucleotide lacks a termination element. In some embodiments, the circular polyribonucleotide lacks an internal ribosomal entry site. In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein. In some embodiments, the circular polyribonucleotide lacks a 5′ cap.

### Production of Circular Polyribonucleotides

Circular polyribonucleotides may be prepared according to any available technique, including, but not limited to, recombinant technology and chemical synthesis. For example, a DNA molecule used to produce a circular RNA molecule can include a DNA sequence of a naturally occurring original nucleic acid sequence, a modified version thereof, or a DNA sequence encoding a synthetic polypeptide not normally found in nature (e.g., chimeric molecules or fusion proteins). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification or mutagenesis of selected regions of a nucleic acid sequence, synthesis of oligonucleotide mixtures and ligation of mixture groups to “build” a mixture of nucleic acid molecules and combinations thereof.

In some embodiments, a linear polyribonucleotide for circularization may be cyclized, or concatemerized. In some embodiments, the linear polyribonucleotide for circularization may be cyclized in vitro prior to formulation and/or delivery. In some embodiments, the circular polyribonucleotide may be in a mixture with linear polyribonucleotides. In some embodiments, the linear polyribonucleotides have the same nucleic acid sequence as the circular polyribonucleotides.

In some embodiments, a linear polyribonucleotide for circularization is cyclized, or concatemerized using a chemical method to form a circular polyribonucleotide. In some chemical methods, the 5′-end and the 3-end of the nucleic acid (e.g., a linear polyribonucleotide for circularization) includes chemically reactive groups that, when close together, may form a new covalent linkage between the 5′-end and the 3-end of the molecule. The 5′-end may contain an NHS-ester reactive group and the 3-end may contain a 3-amino-terminated nucleotide such that in an organic solvent the 3-amino-terminated nucleotide on the 3-end of a linear RNA molecule will undergo a nucleophilic attack on the 5′-NHS-ester moiety forming a new 5′-/3′-amide bond. Other chemical methods of circularization include, but are not limited to click chemistry (e.g., alkyne and azide-based methods, or clickable bases), olefin metathesis, phosphoramidate ligation, hemiaminal-imine crosslinking, base modification, and any combination thereof.

In some embodiments, a linear primary construct or linear polyribonucleotide may be cyclized or concatenated to create a circular polyribonucleotide through methods such as, e.g., chemical, enzymatic, splint ligation, or ribozyme-catalyzed methods. The newly formed 5′-3′ linkage may be an intramolecular linkage or an intermolecular linkage. For example, a splint ligase, such as a SplintR® ligase, RNA ligase II, T4 RNA ligase, or T4 DNA ligase, can be used for splint ligation. According to this method, a single stranded polynucleotide (splint), such as a single-stranded DNA or RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular RNA. In some embodiments, a DNA or RNA ligase may be used in the synthesis of the circular polynucleotides. As a non-limiting example, the ligase may be a circ ligase or circular ligase.

In another example, either the 5′ or 3′ end of the linear polyribonucleotide can encode a ligase ribozyme sequence such that during in vitro transcription, the resultant linear circRNA includes an active ribozyme sequence capable of ligating the 5′ end of the linear polyribonucleotide to the 3′ end of the linear polyribonucleotide. The ligase ribozyme may be derived from the Group I Intron, Hepatitis Delta Virus, Hairpin ribozyme or may be selected by SELEX (systematic evolution of ligands by exponential enrichment).

In another example, a linear polyribonucleotide may be cyclized or concatenated by using at least one non-nucleic acid moiety. For example, the at least one non-nucleic acid moiety may react with regions or features near the 5′ terminus or near the 3′ terminus of the linear polyribonucleotide in order to cyclize or concatenate the linear polyribonucleotide. In another example, the at least one non-nucleic acid moiety may be located in or linked to or near the 5′ terminus or the 3′ terminus of the linear polyribonucleotide. The non-nucleic acid moieties may be homologous or heterologous. As a non-limiting example, the non-nucleic acid moiety may be a linkage such as a hydrophobic linkage, ionic linkage, a biodegradable linkage or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In another example, linear polyribonucleotides may be cyclized or concatenated by self-splicing. In some embodiments, the linear polyribonucleotides may include loop E sequence to self-ligate. In another embodiment, the linear polyribonucleotides may include a self-circularizing intron, e.g., a 5′ and 3′ slice junction, or a self-circularizing catalytic intron such as a Group I, Group II or Group III Introns. Nonlimiting examples of group I intron self-splicing sequences may include self-splicing permuted intron-exon sequences derived from T4 bacteriophage gene td, and the intervening sequence (IVS) rRNA of Tetrahymena, cyanobacterium Anabaena pre-tRNA-Leu gene, or a Tetrahymena pre-rRNA.

In some embodiments, the polyribonucleotide includes catalytic intron fragments, such as a 3′ half of Group I catalytic intron fragment and a 5′ half of Group I catalytic intron fragment. The first and second annealing regions may be positioned within the catalytic intron fragments. Group I catalytic introns are self-splicing ribozymes that catalyze their own excision from mRNA, tRNA, and rRNA precursors via two-metal ion phorphoryl transfer mechanism. Importantly, the RNA itself self-catalyzes the intron removal without the requirement of an exogenous enzyme, such as a ligase.

In some embodiments, the 3′ half of Group I catalytic intron fragment and the 5′ half of Group I catalytic intron fragment are from a cyanobacterium Anabaena pre-tRNA-Leu gene, or a Tetrahymena pre-rRNA.

In some embodiments, the 3′ half of Group I catalytic intron fragment and the 5′ half of Group I catalytic intron fragment are from a Cyanobacterium Anabaena pre-tRNA-Leu gene, and the 3′ exon fragment includes the first annealing region and the 5′ exon fragment includes the second annealing region. The first annealing region may include, e.g., from 5 to 50, e.g., from 10 to 15 (e.g., 10, 11, 12, 13, 14, or 15) ribonucleotides and the second annealing region may include, e.g., from 5 to 50, e.g., from 10 to 15 (e.g., 10, 11, 12, 13, 14, or 15) ribonucleotides.

In some embodiments, the 3′ half of Group I catalytic intron fragment and the 5′ half of Group I catalytic intron fragment are from a Tetrahymena pre-rRNA, and the 3′ half of Group I catalytic intron fragment includes the first annealing region and the 5′ exon fragment includes the second annealing region. In some embodiments, the 3′ exon includes the first annealing region and the 5′ half of Group I catalytic intron fragment includes the second annealing region. The first annealing region may include, e.g., from 6 to 50, e.g., from 10 to 16 (e.g., 10, 11, 12, 13, 14, 15, or 16) ribonucleotides, and the second annealing region may include, e.g., from 6 to 50, e.g., from 10 to 16 (e.g., 10, 11, 12, 13, 14, 15, or 16) ribonucleotides.

In some embodiments, the 3′ half of Group I catalytic intron fragment and the 5′ half of Group I catalytic intron fragment are from a cyanobacterium Anabaena pre-tRNA-Leu gene, a Tetrahymena pre-rRNA, or a T4 phage td gene.

In some embodiments, the 3′ half of Group I catalytic intron fragment and the 5′ Group I catalytic intron fragment are from a T4 phage td gene. The 3′ exon fragment may include the first annealing region and the 5′ half of Group I catalytic intron fragment may include the second annealing region. The first annealing region may include, e.g., from 2 to 16, e.g., 10 to 16 (e.g., 2, 3, 4, 5, 6, 7, 8, 9, 10, 11, 12, 13, 14, 15, or 16) ribonucleotides, and the second annealing region may include, e.g., from 2 to 16, e.g., 10 to 16 (e.g., 2, 3, 4, 5, 6, 7, 8, 9, 10, 11, 12, 13, 14, 15, or 16) ribonucleotides.

In some embodiments, the Group I catalytic intron fragment is from the T4 phage nrdB gene or nrdD gene. In some embodiments, the 5′ half of Group I catalytic intron fragment is from the T4 phage nrdB gene. In some embodiments, the 3′ half of Group I catalytic intron fragment is from the T4 phage nrdB gene and the 5′ half of Group I catalytic intron fragment is from the T4 phage nrdB gene. In some embodiments, the 5′ half of Group I catalytic intron fragment is from the T4 phage nrdD gene. In some embodiments, the 3′ half of Group I catalytic intron fragment is from the T4 phage nrdD gene and the 5′ half of Group I catalytic intron fragment is from the T4 phage nrdD gene.

The 3′ exon fragment may include the first annealing region and the 5′ half of Group I catalytic intron fragment may include the second annealing region. The first annealing region may include, e.g., from 2 to 16, e.g., 10 to 16 (e.g., 2, 3, 4, 5, 6, 7, 8, 9, 10, 11, 12, 13, 14, 15, or 16) ribonucleotides, and the second annealing region may include, e.g., from 2 to 16, e.g., 10 to 16 (e.g., 2, 3, 4, 5, 6, 7, 8, 9, 10, 11, 12, 13, 14, 15, or 16) ribonucleotides.

In some embodiments, the 3′ half of Group I catalytic intron fragment is the 5′ terminus of the linear polynucleotide.

In some embodiments, the 5′ half of Group I catalytic intron fragment is the 3′ terminus of the linear polyribonucleotide.

In another example, a linear polyribonucleotide may be cyclized or concatenated by a non-nucleic acid moiety that causes an attraction between atoms, molecular surfaces at, near, or linked to the 5′ and 3′ ends of the linear polyribonucleotide. The one or more linear polyribonucleotides may be cyclized or concatenated by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In another example, the linear polyribonucleotide may include a ribozyme RNA sequence near the 5′ terminus and near the 3′ terminus. The ribozyme RNA sequence may covalently link to a peptide when the sequence is exposed to the remainder of the ribozyme. The peptides covalently linked to the ribozyme RNA sequence near the 5′ terminus and the 3′terminus may associate with each other, thereby causing a linear polyribonucleotide to cyclize or concatenate. In another example, the peptides covalently linked to the ribozyme RNA near the 5′ terminus and the 3′ terminus may cause the linear primary construct or linear mRNA to cyclize or concatenate after being subjected to ligated using various methods known in the art such as, but not limited to, protein ligation. Non-limiting examples of ribozymes for use in the linear primary constructs or linear polyribonucleotides of the present invention or a non-exhaustive listing of methods to incorporate or covalently link peptides are described in US Patent Publication No. US20030082768, the contents of which is here in incorporated by reference in its entirety.

Methods of making the circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); Muller and Appel, from RNA Biol, 2017, 14(8):1018-1027; and Egli & Herdewijn, Chemistry and Biology of Artificial Nucleic Acids, (First Edition), Wiley-VCH (2012). Other methods of making circular polyribonucleotides are described, for example, in International Publication No. WO2023/044006, International Publication No. WO2022/247943, U.S. Pat. No. 11,000,547, International Publication No. WO2018/191722, International Publication No. WO2019/236673, International Publication No. WO2020/023595, International Publication No. WO2022/204460, International Publication No. WO2022/204464, International Publication No. WO2022/204466, and International Publication No. 2022/261490, the contents of each of which are herein incorporated by reference in their entirety).

Additional methods of synthesizing circular polyribonucleotides are also described elsewhere (see, e.g., U.S. Pat. Nos. 6,210,931, 5,773,244, 5,766,903, 5,712,128, 5,426,180, US Publication No. US20100137407, International Publication No. WO1992001813, International Publication No. WO2010084371, and Petkovic et al., Nucleic Acids Res. 43:2454-65 (2015); the contents of each of which are herein incorporated by reference in their entirety).

In some embodiments, the circular polyribonucleotide is purified, e.g., free ribonucleic acids, linear or nicked RNA, DNA, proteins, etc. are removed. In some embodiments, the circular polyribonucleotides may be purified by any known method commonly used in the art. Examples of nonlimiting purification methods include, column chromatography, gel excision, size exclusion, etc.

### Methods of Production in a Cell-Free System

In some embodiments, circular polyribonucleotides described herein may be produced by transcribing a deoxyribonucleotide template in a cell-free system (e.g., by in vitro transcription) to produce a linear polyribonucleotide. The linear polyribonucleotide produces a splicing-compatible polyribonucleotide, which may be self-spliced to produce a circular polyribonucleotide.

In some embodiments, a circular polyribonucleotide (e.g., in a cell-free system) is produced by providing a linear polyribonucleotide; and self-splicing linear polyribonucleotide under conditions suitable for splicing of the 3′ and 5′ splice sites of the linear polyribonucleotide; thereby producing a circular polyribonucleotide.

In some embodiments, a circular polyribonucleotide is produced by providing a deoxyribonucleotide encoding the linear polyribonucleotide; transcribing the deoxyribonucleotide in a cell-free system to produce the linear polyribonucleotide; optionally purifying the splicing-compatible linear polyribonucleotide; and self-splicing the linear polyribonucleotide under conditions suitable for splicing of the 3′ and 5′ splice sites of the linear polyribonucleotide, thereby producing a circular polyribonucleotide.

In some embodiments, a circular polyribonucleotide is produced by providing a deoxyribonucleotide encoding a linear polyribonucleotide; transcribing the deoxyribonucleotide in a cell-free system to produce the linear polyribonucleotide, wherein the transcribing occurs in a solution under conditions suitable for splicing of the 3′ and 5′ splice sites of the linear polyribonucleotide, thereby producing a circular polyribonucleotide. In some embodiments, the linear polyribonucleotide comprises a 5′ split-intron and a 3′ split-intron (e.g., a self-splicing construct for producing a circular polyribonucleotide). In some embodiments, the linear polyribonucleotide comprises a 5′ annealing region and a 3′ annealing region.

Suitable conditions for in vitro transcriptions and or self-splicing may include any conditions (e.g., a solution or a buffer, such as an aqueous buffer or solution) that mimic physiological conditions in one or more respects. In some embodiments, suitable conditions include between 0.1-100 mM Mg2+ ions or a salt thereof (e.g., 1-100 mM, 1-50 mM, 1-20 mM, 5-50 mM, 5-20 mM, or 5-15 mM). In some embodiments, suitable conditions include between 1-1000 mM K+ ions or a salt thereof such as KCl (e.g., 1-1000 mM, 1-500 mM, 1-200 mM, 50-500 mM, 100-500 mM, or 100-300 mM). In some embodiments, suitable conditions include between 1-1000 mM Cl− ions or a salt thereof such as KCl (e.g., 1-1000 mM, 1-500 mM, 1-200 mM, 50-500 mM, 100-500 mM, or 100-300 mM). In some embodiments, suitable conditions include between 0.1-100 mM Mn2+ ions or a salt thereof such as MnCl2 (e.g., 0.1-100 mM, 0.1-50 mM, 0.1-20 mM, 0.1-10 mM, 0.1-5 mM, 0.1-2 mM, 0.5-50 mM, 0.5-20 mM, 0.5-15 mM, 0.5-5 mM, 0.5-2 mM, or 0.1-10 mM). In some embodiments, suitable conditions include dithiothreitol (DTT) (e.g., 1-1000 μM, 1-500 μM, 1-200 μM, 50-500 μM, 100-500 μM, 100-300 μM, 0.1-100 mM, 0.1-50 mM, 0.1-20 mM, 0.1-10 mM, 0.1-5 mM, 0.1-2 mM, 0.5-50 mM, 0.5-20 mM, 0.5-15 mM, 0.5-5 mM, 0.5-2 mM, or 0.1-10 mM). In some embodiments, suitable conditions include between 0.1 mM and 100 mM ribonucleoside triphosphate (NTP) (e.g., 0.1-100 mM, 0.1-50 mM, 0.1-10 mM, 1-100 mM, 1-50 mM, or 1-10 mM). In some embodiments, suitable conditions include a pH of 4 to 10 (e.g., pH of 5 to 9, pH of 6 to 9, or pH of 6.5 to 8.5). In some embodiments, suitable conditions include a temperature of 4° C. to 50° C. (e.g., 10° C. to 40° C., 15° C. to 40° C., 20° C. to 40° C., or 30° C. to 40° C.),

In some embodiments the linear polyribonucleotide is produced from a deoxyribonucleic acid, e.g., a deoxyribonucleic acid described herein, such as a DNA vector, a linearized DNA vector, or a cDNA. In some embodiments, the linear polyribonucleotide is transcribed from the deoxyribonucleic acid by transcription in a cell-free system (e.g., in vitro transcription).

### Methods of Production in a Cell

In some embodiments circular polyribonucleotides are produced in a cell, e.g., a prokaryotic cell or a eukaryotic cell. In some embodiments, an exogenous polyribonucleotide is provided to a cell (e.g., a linear polyribonucleotide or a DNA molecule encoding for the transcription of a linear polyribonucleotide). The linear polyribonucleotides may be transcribed in the cell from an exogenous DNA molecule provided to the cell. The linear polyribonucleotide may be transcribed in the cell from an exogenous recombinant DNA molecule transiently provided to the cell. In one embodiment, the linear polyribonucleotide may be transcribed in the cell from an exogenous DNA molecule provided to the cell, such as for example, with a plasmid. In some embodiments, the exogenous DNA molecule does not integrate into the cell's genome. In some embodiments, the linear polyribonucleotide is transcribed in the cell from a recombinant DNA molecule that is incorporated into the cell's genome.

In some embodiments, the cell is a prokaryotic cell. In some embodiments, the prokaryotic cell including the polyribonucleotides described herein may be a bacterial cell or an archaeal cell. For example, the prokaryotic cell including the polyribonucleotides described herein may be E. coli, halophilic archaea (e.g., Haloferax volcanii), Sphingomonas, cyanobacteria (e.g., Synechococcus elongatus, Spirulina (Arthrospira) spp., and Synechocystis spp.), Streptomyces, actinomycetes (e.g., Nonomuraea, Kitasatospora, or Thermobifida), Bacillus spp. (e.g., Bacillus subtilis, Bacillus anthracis, Bacillus cereus), betaproteobacteria (e.g., Burkholderia), alphaproteobacterial (e.g., Agrobacterium), Pseudomonas (e.g., Pseudomonas putida), and enterobacteria. The prokaryotic cells may be grown in a culture medium. The prokaryotic cells may be contained in a bioreactor.

In some embodiments, the cell is a eukaryotic cell. In some embodiments, the eukaryotic cell including the polyribonucleotides described herein is a unicellular eukaryotic cell. In some embodiments, the unicellular eukaryotic is a unicellular fungal cell such as a yeast cell (e.g., Saccharomyces cerevisiae and other Saccharomyces spp., Brettanomyces spp., Schizosaccharomyces spp., Torulaspora spp, and Pichia spp.). In some embodiments, the unicellular eukaryotic cell is a unicellular animal cell. A unicellular animal cell may be a cell isolated from a multicellular animal and grown in culture, or the daughter cells thereof. In some embodiments, the unicellular animal cell may be dedifferentiated. In some embodiments, the unicellular eukaryotic cell is a unicellular plant cell. A unicellular plant cell may be a cell isolated from a multicellular plant and grown in culture, or the daughter cells thereof. In some embodiments, the unicellular plant cell may be dedifferentiated. In some embodiments, the unicellular plant cell is from a plant callus. In embodiments, the unicellular cell is a plant cell protoplast. In some embodiments, the unicellular eukaryotic cell is a unicellular eukaryotic algal cell, such as a unicellular green alga, a diatom, a euglenid, or a dinoflagellate. Non-limiting examples of unicellular eukaryotic algae of interest include Dunaliella salina, Chlorella vulgaris, Chlorella zofingiensis, Haematococcus pluvialis, Neochloris oleoabundans and other Neochloris spp., Protosiphon botryoides, Botryococcus braunii, Cryptococcus spp., Chlamydomonas reinhardtii and other Chlamydomonas spp. In some embodiments, the unicellular eukaryotic cell is a protist cell. In some embodiments, the unicellular eukaryotic cell is a protozoan cell.

In some embodiments, the eukaryotic cell is a cell of a multicellular eukaryote. For example, the multicellular eukaryote may be selected from the group consisting of a vertebrate animal, an invertebrate animal, a multicellular fungus, a multicellular alga, and a multicellular plant. In some embodiments, the eukaryotic organism is a human. In some embodiments, the eukaryotic organism is a non-human vertebrate animal. In some embodiments, the eukaryotic organism is an invertebrate animal. In some embodiments, the eukaryotic organism is a multicellular fungus. In some embodiments, the eukaryotic organism is a multicellular plant. In embodiments, the eukaryotic cell is a cell of a human or a cell of a non-human mammal such as a non-human primate (e.g., monkeys, apes), ungulate (e.g., bovids including cattle, buffalo, bison, sheep, goat, and musk ox; pig; camelids including camel, llama, and alpaca; deer, antelope; and equids including horse and donkey), carnivore (e.g., dog, cat), rodent (e.g., rat, mouse, guinea pig, hamster, squirrel), or lagomorph (e.g., rabbit, hare). In embodiments, the eukaryotic cell is a cell of a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the eukaryotic cell is a cell of an arthropod (e.g., insects, arachnids, crustaceans), a nematode, an annelid, a helminth, or a mollusk. In embodiments, the eukaryotic cell is a cell of a multicellular plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the eukaryotic cell is a cell of a eukaryotic multicellular alga.

The eukaryotic cells may be grown in a culture medium. The eukaryotic cells may be contained in a bioreactor.

### Methods of Purification

One or more purification steps may be included in the methods described herein. For example, in some embodiments, the linear polyribonucleotide is substantively enriched or pure (e.g., purified) prior to self-splicing the linear polyribonucleotide. In other embodiments, the linear polyribonucleotide is not purified prior to self-splicing the linear polyribonucleotide. In some embodiments, the resulting circular polyribonucleotide is purified.

Purification may include separating or enriching the desired reaction product from one or more undesired components, such as any unreacted stating material, byproducts, enzymes, or other reaction components. For example, purification of linear polyribonucleotide following transcription in a cell-free system (e.g., in vitro transcription) may include separation or enrichment from the DNA template prior to self-splicing the linear polyribonucleotide. Purification of the circular polyribonucleotide product following splicing may be used to separate or enrich the circular polyribonucleotide from its corresponding linear polyribonucleotide. Methods of purification of RNA are known to those of skill in the art and include enzymatic purification or by chromatography.

In some embodiments, the methods of purification result in a circular polyribonucleotide that has less than 50% (e.g., less than 40%, 30%, 20%, 10%, 5%, 4%, 3%, 2%, or 1%) linear polyribonucleotides.

### Bioreactors

In some embodiments, any method of producing a circular polyribonucleotide described herein may be performed in a bioreactor. A bioreactor refers to any vessel in which a chemical or biological process is carried out which involves organisms or biochemically active substances derived from such organisms. Bioreactors may be compatible with the cell-free methods for production of circular polyribonucleotides described herein. A vessel for a bioreactor may include a culture flask, a dish, or a bag that may be single use (disposable), autoclavable, or sterilizable. A bioreactor may be made of glass, or it may be polymer-based, or it may be made of other materials.

Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor may be a batch or continuous processes. A bioreactor is continuous when the reagent and product streams are continuously being fed and withdrawn from the system. A batch bioreactor may have a continuous recirculating flow, but no continuous feeding of reagents or product harvest.

Some methods of the present disclosure are directed to large-scale production of circular polyribonucleotides. For large-scale production methods, the method may be performed in a volume of 1 liter (L) to 50 L, or more (e.g., 5 L, 10 L, 15 L, 20 L, 25 L, 30 L, 35 L, 40 L, 45 L, 50 L, or more). In some embodiments, the method may be performed in a volume of 5 L to 10 L, 5 L to 15 L, 5 L to 20 L, 5 L to 25 L, 5 L to 30 L, 5 L to 35 L, 5 L to 40 L, 5 L to 45 L, 10 L to 15 L, 10 L to 20 L, 10 L to 25 L, 20 L to 30 L, 10 L to 35 L, 10 L to 40 L, 10 L to 45 L, 10 L to 50 L, 15 L to 20 L, 15 L to 25 L, 15 L to 30 L, 15 L to 35 L, 15 L to 40 L, 15 L to 45 L, or 15 to 50 L.

In some embodiments, a bioreactor may produce at least 1 g of circular polyribonucleotide. In some embodiments, a bioreactor may produce 1-200 g of circular polyribonucleotide (e.g., 1-10 g, 1-20 g, 1-50 g, 10-50 g, 10-100 g, 50-100 g, or 50-200 g of circular RNA). In some embodiments, the amount produced is measured per liter (e.g., 1-200 g per liter), per batch or reaction (e.g., 1-200 g per batch or reaction), or per unit time (e.g., 1-200 g per hour or per day).

In some embodiments, more than one bioreactor may be utilized in series to increase the production capacity (e.g., one, two, three, four, five, six, seven, eight, or nine bioreactors may be used in series).

### Gene Editing

In some instances, the LNMP/nucleic acid vaccines may include a component of a gene editing system. For example, the agent may introduce an alteration (e.g., insertion, deletion (e.g., knockout), translocation, inversion, single point mutation, or other mutation) in a gene in the target organism. Exemplary gene editing systems include the zinc finger nucleases (ZFNs), Transcription Activator-Like Effector-based Nucleases (TALEN), and the clustered regulatory interspaced short palindromic repeat (CRISPR) system. ZFNs, TALENs, and CRISPR-based methods are described, e.g., in Gaj et al., Trends Biotechnol. 31(7):397-405, 2013.

Additional descriptions about the component and process of the gene editing system may be found in International Patent Application Publication No. WO 2021/041301, which is incorporated herein by reference in its entirety.

### Nucleic Acid Vaccine for Viral Infection

The LNMP/nucleic acid vaccine comprises one or more polynucleotides (e.g., mRNA or circRNA) encoding one or more antigenic or signaling polypeptides for therapeutic purpose, such as to combat various viral infections. The one or more polynucleotides (e.g., mRNA or circRNA) encode one or more antigenic polypeptides derived from an infectious agent that causes an infectious disease, disorder, or condition, e.g., a viral infection caused by an RNA virus. In some embodiments, the LNMP/nucleic acid vaccine comprises one or more circular polyribonucleotides. In some embodiments, the circular polyribonucleotide includes one or more polyribonucleotide (e.g., one or more polyribonucleotide cargo of the polyribonucleotide) encoding one or more antigenic or signaling polypeptides for therapeutic purpose, such as to combat various viral infections. In some embodiments, the polyribonucleotide includes one or more expression sequences encoding one or more antigenic or signaling polypeptides derived from an infectious agent that causes an infectious disease, disorder, or condition, e.g., a viral infection caused by an RNA virus

In some embodiments, the polynucleotide (e.g., mRNA or circRNA) in the LNMP/nucleic acid vaccine encodes one or more wild type or engineered antigens (or an antibody to an antigen) of the various types and strains of virus as described below.

### Viral Infection.

The LNMP/nucleic acid vaccine may be suitable to combat the infectious diseases, disorders, or conditions associated with viral infections including, but not limited to, acute febrile pharyngitis, pharyngoconjunctival fever, epidemic keratoconjunctivitis, infantile gastroenteritis, coxsackie infections, infectious mononucleosis, burkitt lymphoma, acute hepatitis, chronic hepatitis, hepatic cirrhosis, hepatocellular carcinoma, primary HSV-1 infection (e.g., gingivostomatitis in children, tonsillitis and pharyngitis in adults, keratoconjunctivitis), latent HSV-1 infection (e.g., herpes labialis and cold sores), primary HSV-2 infection, latent HSV-2 infection, aseptic meningitis, infectious mononucleosis, cytomegalic inclusion disease, kaposi sarcoma, multicentric castleman disease, primary effusion lymphoma, AIDS, influenza, reye syndrome, measles, postinfectious encephalomyelitis, Mumps, hyperplastic epithelial lesions (e.g., common, flat, plantar and anogenital warts, laryngeal papillomas, epidermodysplasia verruciformis), cervical carcinoma, squamous cell carcinomas, croup, pneumonia, bronchiolitis, common cold, poliomyelitis, rabies, bronchiolitis, pneumonia, influenza-like syndrome, severe bronchiolitis with pneumonia, german measles, congenital rubella, varicella, and herpes zoster.

Exemplary viral infectious agents include, but are not limited to, a strain of virus selected from the group consisting of: adenovirus; Herpes simplex, type 1; Herpes simplex, type 2; encephalitis virus, papillomavirus, Varicella-zoster virus; Epstein-barr virus; Human cytomegalovirus; Human herpesvirus, type 8; Human papillomavirus; BK virus; JC virus; Smallpox; polio virus, Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; yellow fever virus; dengue virus; West Nile virus; Rubella virus; Hepatitis E virus; Human immunodeficiency virus (HIV); Influenza virus, type A or B; Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytial virus; Human metapneumovirus; Hendra virus; Nipah virus; Rabies virus; Hepatitis D; Rotavirus; Orbivirus; Coltivirus; Hantavirus, Middle East Respiratory Coronavirus; Chikungunya virus or Banna virus.

The infectious agent may be a strain of virus selected from the group consisting of the virus from the following table.

Adenovirus
Banna virus
BK virus
Chikungunya Virus
Coltivirus
coxsackievirus
Crimean-Congo hemorrhagic fever virus
Dengue virus
Ebola virus
Encephalitis virus
Japanese encephalitis virus
Eastern equine encephalitis
Epstein-barr virus
Guanarito virus
Hanta virus
Herpes simplex, type 1
Herpes simplex, type 2
Human herpes virus, type 8
Herpes zoster (Varicella-zoster; Shingles) virus
Hepatitis A virus
Hepatitis B virus
Hepatitis C virus
Hepatitis D
Hepatitis E virus
Hendra virus
Human astrovirus
Human bocavirus
Human cytomegalovirus
Human Enterovirus
Human Enterovirus 68
Human Enterovirus 71
Human metapneumovirus
Human Immunodeficiency virus (HIV)
Influenza virus
Parainfluenza virus
JC virus
Junin virus
Lassa virus
Machupo virus
Marburg virus
Measles virus
Mumps virus
Nipah virus
Norwalk virus
Orbivirus
papillomavirus
Human papillomavirus (HPV)
Parvovirus B19
polio virus
Rabies virus
Rhinovirus
Rotavirus
Rubella virus
Respiratory syncytial virus (RSV)
Severe acute respiratory syndrome (SARS) virus
Middle East Respiratory Syndrome Corona (MERS) Virus
SARS-COV-2
Smallpox
Sabiá virus
Vesicular exanthernavirus
West Nile virus
Yellow Fever virus
Zika virus

Other suitable viral infections and viral infectious agents are described in U.S. Patent Application Publication No. US2019/0015501 and U.S. Pat. No. 11,007,260, both of which are incorporated by reference in their entirety.

### a Mosquito-Borne Virus

Dengue. In some embodiments, the polynucleotide (e.g., mRNA or circRNA) in the LNMP/nucleic acid vaccine encodes a strain of dengue virus (a flavi virus). In some embodiments, the polynucleotide (e.g., mRNA or circRNA) encodes the E protein domain III (DENV1-4 tandem mRNA), the E protein domain I/II hinge region (DENV1-4 individual mRNAs or circRNAs), the prM protein (DENV1-4 tandem or individual mRNAs or circRNAs) and the C protein (DENV1-4 tandem or single mRNAs or circRNAs).

Chikungunya virus. In some embodiments, the polynucleotide (e.g., mRNA or circRNA) in the LNMP/nucleic acid vaccine encodes a strain of Chikungunya virus. In some embodiments, the antigenic polypeptide encodes Chikungunya envelope and/or capsid antigenic polypeptide selected from the group consisting of C, E1, E2, E3, 6K, and C-E3-E2-6K-E1.

In some embodiments, the polynucleotide (e.g., mRNA or circRNA) in the LNMP/nucleic acid vaccine encodes a Chikungunya polypeptide selected from the following strains and isolates: TA53, SA76, UG82, 37997, IND-06, Ross, S27, M-713424, E1-A226V, E1-T98, IND-63-WB1, Gibbs 63-263, TH35, 1-634029, AF15561, IND-73-MH5, 653496, C0392-95, P0731460, MY0211MR/06/BP, SV0444-95, K0146-95, TSI-GSD-218-VR1, TSI-GSD-218, M127, M125, 6441-88, MY0031MR/06/BP, MY0021MR/06/BP, TR206/H804187, MY/06/37348, MY/06/37350, NC/2011-568, 1455-75, RSU1, 0706aTw, InDRE51CHIK, PR—S4, AMA2798/H804298, Hu/85/NR/001, PhH15483, 0706aTw, 0802aTw, MY0191MR/06/BP, PR—S6, PER160/1H803609, 99659, JKT23574, 0811 aTw, CHIK/SBY6/10, 2001908323-BDG E1, 2001907981-BDG E1, 2004904899-BDG E1, 2004904879-BDG E1, 2003902452-BDG E1, DH 130003, 0804aTw, 2002918310-BDG E1, JC2012, chik-sy, 3807, 3462, Yap 13-2148, PR—S5, 0802aTw, MY0191MR/06/Bp, 0706aTw, PhH15483, Hu/85/NR/001, CHIKV-13-112A, InDRE 4CHIK, 0806aTw, 0712aTw, 3412-78, Yap 13-2039, LEIV-CHIKV/Moscow/1, DH130003, and 20039.

Zika virus. In some embodiments, the polynucleotide (e.g., mRNA or circRNA) in the LNMP/nucleic acid vaccine encodes a strain of Zika virus (a flavi virus). In some embodiments, the polynucleotide (e.g., mRNA or circRNA) in the LNMP/nucleic acid vaccine encodes a ZIKV polypeptide from a ZIKV serotype selected from the group consisting of MR 766, SPH2015, and ACD75819.

Venezuelan equine encephalitis (VEE) virus. In some embodiments, the polynucleotide (e.g., mRNA or circRNA) in the LNMP/nucleic acid vaccine encodes a strain of VEE virus.

The polynucleotide (e.g., mRNA or circRNA) in the LNMP/nucleic acid vaccine can encode additional types of virus and strains of a mosquito-borne virus, or fragments thereof, as those described in U.S. Pat. No. 11,007,260, which is incorporated herein by reference in its entirety.

### Influenza

In some embodiments, the polynucleotide (e.g., mRNA or circRNA) in the LNMP/nucleic acid vaccine encodes a strain of an influenza virus. In some embodiments, the polynucleotide (e.g., mRNA or circRNA) in the LNMP/nucleic acid vaccine encodes a strain of Influenza A, Influenza B, or combinations thereof. In some embodiments, the strain of Influenza A or Influenza B is associated with birds, pigs, horses, dogs, humans or non-human primates.

In some embodiments, the polynucleotide (e.g., mRNA or circRNA) in the LNMP/nucleic acid vaccine encodes a hemagglutinin protein or fragment thereof. In some embodiments, the hemagglutinin protein is H1, H2, H3, H4, H5, H6, H7, H8, H9, H10, H11, H12, H13, H14, H15, H16, H17, H18, or a fragment thereof. In some embodiments, the hemagglutinin protein does not comprise a head domain (HA1). In some embodiments, the hemagglutinin protein comprises a portion of the head domain (HA1). In some embodiments, the hemagglutinin protein does not comprise a cytoplasmic domain. In some embodiments, the hemagglutinin protein comprises a portion of the cytoplasmic domain.

In some embodiments, the polynucleotide (e.g., mRNA or circRNA) in the LNMP/nucleic acid vaccine encodes a truncated hemagglutinin protein. In some embodiments, the truncated hemagglutinin protein comprises a portion of the transmembrane domain. In some embodiments, the virus is selected from the group consisting of H1N1, H3N2, H5N1, H7N9, and H10N8.

The polynucleotide (e.g., mRNA or circRNA) in the LNMP/nucleic acid vaccine can encode additional types of virus and strains of an influenza virus, or fragments thereof, as those described in U.S. Patent Application Publication No. 2019/0015501, which is incorporated herein by reference in its entirety.

### Coronavirus

Betacoronavirus. In some embodiments, the polynucleotide (e.g., mRNA or circRNA) in the LNMP/nucleic acid vaccine encodes a peptide/protein comprising a spike protein (S) of a betacoronavirus (BetaCoV), or a fragment or subunit thereof.

In some embodiments, the polynucleotide (e.g., mRNA or circRNA) in the LNMP/nucleic acid vaccine comprises an open reading frame encoding a peptide/protein comprising a spike protein (S) of a betacoronavirus (BetaCoV), or a fragment or subunit thereof.

The polynucleotide (e.g., mRNA or circRNA) in the LNMP/nucleic acid vaccine can encode different types of betacoronavirus and strains of a betacoronavirus, or fragments thereof, as those described in U.S. Pat. No. 10,933,127, which is incorporated herein by reference in its entirety.

Middle East respiratory syndrome coronavirus. In some embodiments, the polynucleotide (e.g., mRNA or circRNA) in the LNMP/nucleic acid vaccine encodes a peptide/protein comprising a spike protein (S), a spike S1 fragment (S1), an envelope protein (E), a membrane protein (M), and/or a nucleocapsid protein (N) of a MERS coronavirus, or a fragment or variant of any one of these proteins.

The polynucleotide (e.g., mRNA or circRNA) in the LNMP/nucleic acid vaccine can encode different strains of a MERS coronavirus, or fragments thereof, as those described in U.S. Patent Application Publication No. US2019/0351048, which is incorporated herein by reference in its entirety.

### SARS-CoV-2

In some embodiments, the polynucleotide (e.g., mRNA or circRNA) in the LNMP/nucleic acid vaccine encodes one or more wild type or engineered antigens (or an antibody to an antigen) of SARS-CoV-2.

In some embodiments, the open reading frame of the polynucleotide (e.g., mRNA or circRNA) in the LNMP/nucleic acid vaccine encodes one or more wild type or engineered antigens (or an antibody to an antigen) of SARS-CoV-2. In some embodiments, the open reading frame is codon-optimized.

In some embodiments, the polynucleotide (e.g., mRNA or circRNA) in the LNMP/nucleic acid vaccine encodes a peptide/protein comprising a spike protein (S), a membrane (M) protein, an envelope (E) protein, and/or a nucleocapsid (NC) protein of a SARS-CoV-2 virus, or a fragment or variant thereof.

In some embodiments, the polynucleotide (e.g., mRNA or circRNA) in the LNMP/nucleic acid vaccine encodes a peptide/protein comprising a spike protein (S) of a SARS-CoV-2 virus, or a fragment or variant thereof. In some embodiments, the polynucleotide (e.g., mRNA or circRNA) in the LNMP/nucleic acid vaccine codes a peptide/protein comprising at least one or two domains a spike protein (S) of a SARS-CoV-2 virus, and less than the full-length spike protein.

In some embodiments, the polynucleotide (e.g., mRNA or circRNA) in the LNMP/nucleic acid vaccine comprises an open reading frame (ORF) that encodes a SARS-CoV-2 spike (S) protein having a double proline stabilizing mutation.

In some embodiments, the polynucleotide (e.g., mRNA or circRNA) in the LNMP/nucleic acid vaccine (or its open reading frame) encodes a strain or variant of SARS-CoV-2 virus selected from the group consisting of:
alpha (lineage B.1.1.7, Q.1-Q.8),
beta (lineage B.1.351, B.1.351.2, B.1.351.3),
delta
gamma (lineage P.1, P.1.1, P.1.2)
epsilon (lineage B.1.427, B.1.429)
eta (lineage B.1.525)
iota (lineage B.1.526)
kappa (lineage B.1.617.1)
B.1.617.3
Lambda (lineage C.37)
mu (lineage B.1.621, B.1.621.1)
zeta (lineage P.2).
omicron

In some embodiments, the polynucleotide (e.g., mRNA or circRNA) in the LNMP/nucleic acid vaccine (or its open reading frame) encodes a SARS-CoV-2 spike (S) protein and/or RBD, or fragments thereof. In some embodiments, the S antigen and/or RBD antigen fragment thereof comprise one or more mutations within the RBD selected from the group consisting of: K417N or K417T, N439N, N440K, G446V, L452R, Y453F, S477G or S477N, E484Q or E484K, F490S, N501S or N501Y, D614G, Q677P or Q677H, P681H or P681R.

In some embodiments, the polynucleotide (e.g., mRNA or circRNA) in the LNMP/nucleic acid vaccine (or its open reading frame) encodes a SARS-CoV-2 spike (S) protein and/or RBD, or fragments thereof. In some embodiments, the S antigen comprises a mutation which stabilizes the Spike trimer, including e.g., the K986P and V987P mutations (S-2P variant) and other proline substitutions, in particular F817P, A892P, A899P and A942P, which can be combined together to obtain a multiple proline variant, in particular hexaproline variant (HexaPro).

In some embodiments, the polynucleotide (e.g., mRNA or circRNA) in the LNMP/nucleic acid vaccine (or its open reading frame) encodes a SARS-CoV-2 spike (S) protein and/or RBD, or fragments thereof. In some embodiments, the S antigen comprises one or more mutations selected from the group consisting of: the substitutions L18F, T20N, P26S, D80A, D138Y, R190S, D215G, A570D, D614G, H655Y, P681H, A701V, T7161, S982A, T10271, D1118H and V1176F; and the deletions delta 69-70, delta 144, delta 242-244, and delta 246-252.

In some embodiments, the polynucleotide (e.g., mRNA or circRNA) in the LNMP/nucleic acid vaccine (or its open reading frame) encodes a SARS-CoV-2 spike (S) protein and/or RBD, or fragments thereof. In some embodiments, the S antigen or RBD antigen fragment thereof comprises the following mutations: N501Y; E484K and N501Y; K417T or K417N, E484K and N501Y; K417N, N439N, Y453F, S477N, E484K, F490S, and N501Y; K417N, N439N, L452R, S477N, E484K, F490S, and N501Y.

Additional mutations may be found in WO 2021/154763A1, which is incorporated by reference in its entirety.

In some embodiments, the polynucleotide in the LNMP/nucleic acid vaccine (or its open reading frame) is a circRNA comprising the following sequence or a fragment thereof:

(SEQ ID NO: 26)
AAAATCCGTAGCGTCTAAACGGTCGTGTGGGTTCAAGTCCCTCCACCCCCAAAAAAAATAAAAAA
AAATTAAAAAAAAAATTTAAAAAAAAATTAAAAAAATTTAAAAAAAATTAAAAAAAAAATAAAAAATT
AAAAAAATAAAAAAAAAATTTAAAAAAAAATAAAAAAACACATTAAAACAGCCTGTGGGTTGATCCC
ACCCACAGGCCCATTGGGCGCTAGCACTCTGGTATCACGGTACCTTTGTGCGCCTGTTTTATAC
CCCCTCCCCCAACTGTAACTTAGAAGTAACACACACCGATCAACAGTCAGCGTGGCACACCAGC
CACGTTTTGATCAAGCACTTCTGTTACCCCGGACTGAGTATCAATAGACTGCTCACGCGGTTGAA
GGAGAAAGCGTTCGTTATCCGGCCAACTACTTCGAAAAACCTAGTAACACCGTGGAAGTTGCAG
AGTGTTTCGCTCAGCACTACCCCAGTGTAGATCAGGTCGATGAGTCACCGCATTCCCCACGGGC
GACCGTGGCGGTGGCTGCGTTGGCGGCCTGCCCATGGGGAAACCCATGGGACGCTCTAATACA
GACATGGTGCGAAGAGTCTATTGAGCTAGTTGGTAGTCCTCCGGCCCCTGAATGCGGCTAATCC
TAACTGCGGAGCACACACCCTCAAGCCAGAGGGCAGTGTGTCGTAACGGGCAACTCTGCAGCG
GAACCGACTACTTTGGGTGTCCGTGTTTCATTTTATTCCTATACTGGCTGCTTATGGTGACAATTG
AGAGATCGTTACCATATAGCTATTGGATTGGCCATCCGGTGACTAATAGAGCTATTATATATCCCT
TTGTTGGGTTTATACCACTTAGCTTGAAAGAGGTTAAAACATTACAATTCATTGTTAAGTTGAATAC
AGCAACATGTTTGTTTTTCTTGTTTTATTGCCACTAGTCTCTAGTCAGTGTGTTAATCTTACAACCA
GAACTCAATTACCCCCTGCATACACTAATTCTTTCACACGTGGTGTTTATTACCCTGACAAAGTTT
TCAGATCCTCAGTTTTACATTCAACTCAGGATTTGTTCTTACCTTTCTTTTCCAATGTTACTTGGTT
CCATGCTATACATGTCTCTGGGACCAATGGTACTAAGAGGTTTGATAACCCTGTCCTACCATTTAA
TGATGGTGTTTATTTTGCTTCCACTGAGAAGTCTAACATAATAAGAGGCTGGATCTTTGGTACTAC
TTTAGATTCGAAGACCCAGTCCCTACTTATTGTTAATAACGCTACTAATGTTGTTATTAAAGTCTGT
GAATTTCAATTTTGTAATGATCCATTTTTGGGTGTTTATTACCACAAAAACAACAAAAGTTGGATGG
AAAGTGAGTTCAGAGTTTATTCTAGTGCGAATAATTGCACTTTTGAATATGTCTCTCAGCCTTTTCT
TATGGACCTTGAAGGAAAACAGGGTAATTTCAAAAATCTTAGGGAATTTGTGTTTAAGAATATTGA
TGGTTATTTTAAAATATATTCTAAGCACACGCCTATCAACCTCGTCAGGGACCTGCCGCAGGGCT
TCAGCGCTCTGGAGCCTCTGGTGGATCTCCCCATCGGGATCAACATCACGAGGTTCCAGACGCT
GCTGGCCCTGCACAGGTCCTACCTGACCCCGGGCGACAGCAGCTCGGGGTGGACCGCGGGGG
CGGCGGCCTACTACGTGGGCTACCTGCAGCCCAGGACGTTCTTGTTGAAGTACAACGAGAACG
GCACAATCACCGACGCTGTGGACTGCGCTCTGGATCCTCTCTCCGAGACTAAGTGCACCCTGAA
GTCTTTCACCGTGGAGAAGGGGATCTACCAGACCTCCAACTTCAGAGTGCAGCCCACAGAGTCG
ATTGTGCGCTTCCCCAATATCACAAACCTGTGCCCCTTTGGGGAGGTGTTCAACGCCACGCGGT
TCGCGTCCGTGTACGCCTGGAATCGCAAGCGCATTTCCAACTGCGTCGCGGACTACTCGGTGCT
GTACAACTCCGCCTCGTTCTCCACCTTCAAGTGCTACGGAGTGTCTCCGACCAAGCTGAACGAT
CTGTGTTTCACCAACGTGTACGCAGACTCGTTCGTGATCAGGGGGGACGAGGTGAGGCAGATA
GCACCGGGCCAGACGGGGAAGATCGCTGATTACAACTACAAGCTCCCCGACGATTTCACCGGC
TGTGTGATCGCTTGGAACTCCAACAACCTGGATTCCAAGGTCGGGGGGAACTACAACTATCTCTA
TCGACTCTTCAGAAAGAGCAATCTGAAGCCCTTCGAGAGAGATATCAGCACTGAGATCTATCAGG
CTGGGAGCACCCCGTGCAACGGGGGGAGGGGTTCAACTGCTACTTCCCACTGCAATCCTACG
GCTTCCAGCCCACGAACGGCGTGGGCTATCAGCCTTATAGGGTTGTGGTGCTGAGCTTTGAGCT
CCTCCACGCCCCAGCCACCGTCTGTGGACCAAAGAAGTCCACAAATCTGGTGAAAAATAAGTGC
GTGAACTTTAATTTCAACGGACTGACGGGGACTGGGGTGCTGACGGAGAGCAACAAGAAGTTCC
TGCCCTTCCAGCAGTTCGGTCGTGACATCGCCGACACGACCGACGCTGTGAGGGACCCCCAGA
CGCTGGAGATCCTGGACATCACTCCCTGCAGCTTTGGGGGTGTCTCCGTCATCACGCCTGGTAC
CAACACTTCCAACCAGGTGGCAGTGTTGTACCAGGACGTGAACTGCACGGAGGTGCCCGTGGC
AATCCACGCCGACCAGTTGACCCCAACCTGGCGCGTGTATTCCACAGGTAGCAACGTGTTTCAG
ACCCGGGCCGGCTGTCTGATTGGCGCCGAGCACGTGAACAATAGCTACGAGTGCGACATCCCC
ATCGGGGCCGGAATCTGTGCGTCTTACCAGACGCAGACCAATTCCCCCCGGCGGGCTCGCAGC
GTAGCTAGCCAGTCAATCATAGCCTACACGATGTCCCTGGGCGCCGAGAACTCGGTGGCCTATA
GCAATAACTCTATTGCTATCCCTACTAACTTCACGATCTCGGTCACCACCGAGATCCTGCCAGTT
AGTATGACCAAGACCTCCGTGGATTGCACCATGTACATCTGTGGTGATTCCACGGAGTGCAGCA
ACCTGCTGCTGCAGTACGGTTCATTCTGCACCCAGCTGAACCGTGCCCTGACAGGCATCGCCGT
GGAGCAGGACAAGAACACCCAGGAGGTGTTTGCCCAGGTCAAGCAGATCTACAAGACACCTCCT
ATCAAGGACTTCGGCGGATTCAACTTCTCCCAGATCCTCCCGGATCCGTCGAAGCCTTCCAAGC
GGTCCTTCATCGAGGACCTCCTGTTCAACAAGGTGACCCTGGCCGACGCCGGCTTCATCAAGCA
GTACGGCGACTGCCTGGGGGACATCGCGGCCCGGGATCTGATTTGTGCCCAGAAGTTCAACGG
GCTGACTGTGCTTCCGCCTCTGCTGACCGACGAGATGATCGCTCAGTACACCTCCGCCCTGCTG
GCTGGGACCATCACCAGCGGGTGGACGTTCGGGGCTGGAGCCGCGCTCCAGATCCCATTCGC
GATGCAGATGGCATACCGCTTCAACGGAATTGGAGTGACACAGAACGTGCTGTACGAGAACCAG
AAGCTGATCGCGAACCAGTTCAACTCCGCGATCGGCAAGATTCAGGACTCCCTGAGCTCTACCG
CCAGCGCCCTGGGCAAGCTGCAGGACGTGGTCAACCAGAACGCCCAGGCGCTGAACACCCTG
GTGAAGCAGCTGTCCTCCAATTTCGGTGCCATCTCATCCGTGCTGAACGACATCTTGTCTCGGCT
GGACCCTCCCGAGGCGGAGGTGCAGATCGACCGCCTCATCACGGGGCGGCTGCAGTCCCTGC
AGACCTACGTGACTCAGCAGCTCATCAGGGCTGCTGAGATCAGGGCCAGCGCCAATCTGGCAG
CTACCAAGATGTCAGAGTGCGTGCTGGGCCAGAGCAAGAGGGTGGACTTCTGTGGCAAGGGAT
ATCACCTGATGTCCTTTCCACAGAGTGCACCCCATGGTGTGGTGTTCTTGCACGTGACCTACGTG
CCCGCCCAGGAGAAGAACTTCACAACCGCCCCAGCCATCTGCCACGACGGGAAGGCCCACTTC
CCTCGGGAGGGGGTCTTCGTCAGTAACGGTACTCACTGGTTCGTGACCCAGAGGAATTTCTACG
AGCCCCAGATCATCACCACTGACAATACCTTTGTGAGTGGGAACTGCGACGTCGTGATCGGTAT
TGTGAACAATACCGTGTACGACCCGCTGCAGCCTGAGCTGGATTCTTTCAAGGAGGAGCTGGAC
AAGTACTTCAAGAATCACACGTCCCCCGACGTGGATCTGGGGGATATCTCTGGAATCAACGCCT
CTGTGGTCAACATCCAGAAGGAGATCGACAGGCTCAACGAGGTGGCCAAGAACCTCAACGAGA
GCCTGATCGATCTCCAGGAGCTCGGGAAGTACGAGCAGTACATCAAGTGGCCGTGGTACATCTG
GCTGGGGTTCATTGCCGGACTCATCGCCATCGTGATGGTCACGATCATGCTGTGCTGCATGACT
TCCTGCTGCAGCTGCCTCAAGGGCTGCTGCTCTTGCGGCAGCTGCTGCAAGTTCGACGAGGAC
GACTCCGAGCCCGTCCTCAAGGGAGTCAAGCTGCACTACACCTAAATAAAAAAAAATTAAAAAAA
AAATTTAAAAAAAAATTAAAAAAATTTAAAAAAAATTAAAAAAAAAATAAAAAATTAAAAAAATAAAA
AAAAAATTTAAAAAAAAATAAAAAAGCAACCCACACGACCGTTTAGACGCTACGGACTT.

In some embodiments, the polynucleotide in the LNMP/nucleic acid vaccine (or its open reading frame) comprises a linear RNA (a linear version of the circRNA represented by SEQ ID NO: 26) comprising the following sequence or a fragment thereof:

(SEQ ID NO: 27)
GGGAACAACAGATAACTTACAGCTAGTCGGAAGGTGCAGAGACTCGACGGGAGCTACCCTAACG
TCAAGACGAGGGTAAAGAGAGAGTCCAATTCTCAAAGCCAATAGGCAGTAGCGAAAGCTGCGGG
AGAATGAAAATCCGTAGCGTCTAAACGGTCGTGTGGGTTCAAGTCCCTCCACCCCCAAAAAAAAT
AAAAAAAAATTAAAAAAAAAATTTAAAAAAAAATTAAAAAAATTTAAAAAAAATTAAAAAAAAAATAA
AAAATTAAAAAAATAAAAAAAAAATTTAAAAAAAAATAAAAAAACACATTAAAACAGCCTGTGGGTT
GATCCCACCCACAGGCCCATTGGGCGCTAGCACTCTGGTATCACGGTACCTTTGTGCGCCTGTT
TTATACCCCCTCCCCCAACTGTAACTTAGAAGTAACACACACCGATCAACAGTCAGCGTGGCACA
CCAGCCACGTTTTGATCAAGCACTTCTGTTACCCCGGACTGAGTATCAATAGACTGCTCACGCGG
TTGAAGGAGAAAGCGTTCGTTATCCGGCCAACTACTTCGAAAAACCTAGTAACACCGTGGAAGTT
GCAGAGTGTTTCGCTCAGCACTACCCCAGTGTAGATCAGGTCGATGAGTCACCGCATTCCCCAC
GGGCGACCGTGGCGGTGGCTGCGTTGGCGGCCTGCCCATGGGGAAACCCATGGGACGCTCTA
ATACAGACATGGTGCGAAGAGTCTATTGAGCTAGTTGGTAGTCCTCCGGCCCCTGAATGCGGCT
AATCCTAACTGCGGAGCACACACCCTCAAGCCAGAGGGCAGTGTGTCGTAACGGGCAACTCTGC
AGCGGAACCGACTACTTTGGGTGTCCGTGTTTCATTTTATTCCTATACTGGCTGCTTATGGTGAC
AATTGAGAGATCGTTACCATATAGCTATTGGATTGGCCATCCGGTGACTAATAGAGCTATTATATA
TCCCTTTGTTGGGTTTATACCACTTAGCTTGAAAGAGGTTAAAACATTACAATTCATTGTTAAGTTG
AATACAGCAACATGTTTGTTTTTCTTGTTTTATTGCCACTAGTCTCTAGTCAGTGTGTTAATCTTAC
AACCAGAACTCAATTACCCCCTGCATACACTAATTCTTTCACACGTGGTGTTTATTACCCTGACAA
AGTTTTCAGATCCTCAGTTTTACATTCAACTCAGGATTTGTTCTTACCTTTCTTTTCCAATGTTACT
TGGTTCCATGCTATACATGTCTCTGGGACCAATGGTACTAAGAGGTTTGATAACCCTGTCCTACC
ATTTAATGATGGTGTTTATTTTGCTTCCACTGAGAAGTCTAACATAATAAGAGGCTGGATCTTTGG
TACTACTTTAGATTCGAAGACCCAGTCCCTACTTATTGTTAATAACGCTACTAATGTTGTTATTAAA
GTCTGTGAATTTCAATTTTGTAATGATCCATTTTTGGGTGTTTATTACCACAAAAACAACAAAAGTT
GGATGGAAAGTGAGTTCAGAGTTTATTCTAGTGCGAATAATTGCACTTTTGAATATGTCTCTCAGC
CTTTTCTTATGGACCTTGAAGGAAAACAGGGTAATTTCAAAAATCTTAGGGAATTTGTGTTTAAGA
ATATTGATGGTTATTTTAAAATATATTCTAAGCACACGCCTATCAACCTCGTCAGGGACCTGCCGC
AGGGCTTCAGCGCTCTGGAGCCTCTGGTGGATCTCCCCATCGGGATCAACATCACGAGGTTCCA
GACGCTGCTGGCCCTGCACAGGTCCTACCTGACCCCGGGCGACAGCAGCTCGGGGTGGACCG
CGGGGGCGGCGGCCTACTACGTGGGCTACCTGCAGCCCAGGACGTTCTTGTTGAAGTACAACG
AGAACGGCACAATCACCGACGCTGTGGACTGCGCTCTGGATCCTCTCTCCGAGACTAAGTGCAC
CCTGAAGTCTTTCACCGTGGAGAAGGGGATCTACCAGACCTCCAACTTCAGAGTGCAGCCCACA
GAGTCGATTGTGCGCTTCCCCAATATCACAAACCTGTGCCCCTTTGGGGAGGTGTTCAACGCCA
CGCGGTTCGCGTCCGTGTACGCCTGGAATCGCAAGCGCATTTCCAACTGCGTCGCGGACTACTC
GGTGCTGTACAACTCCGCCTCGTTCTCCACCTTCAAGTGCTACGGAGTGTCTCCGACCAAGCTG
AACGATCTGTGTTTCACCAACGTGTACGCAGACTCGTTCGTGATCAGGGGGGACGAGGTGAGGC
AGATAGCACCGGGCCAGACGGGGAAGATCGCTGATTACAACTACAAGCTCCCCGACGATTTCAC
CGGCTGTGTGATCGCTTGGAACTCCAACAACCTGGATTCCAAGGTCGGGGGGAACTACAACTAT
CTCTATCGACTCTTCAGAAAGAGCAATCTGAAGCCCTTCGAGAGAGATATCAGCACTGAGATCTA
TCAGGCTGGGAGCACCCCGTGCAACGGGGTGGAGGGGTTCAACTGCTACTTCCCACTGCAATC
CTACGGCTTCCAGCCCACGAACGGCGTGGGCTATCAGCCTTATAGGGTTGTGGTGCTGAGCTTT
GAGCTCCTCCACGCCCCAGCCACCGTCTGTGGACCAAAGAAGTCCACAAATCTGGTGAAAAATA
AGTGCGTGAACTTTAATTTCAACGGACTGACGGGGACTGGGGTGCTGACGGAGAGCAACAAGAA
GTTCCTGCCCTTCCAGCAGTTCGGTCGTGACATCGCCGACACGACCGACGCTGTGAGGGACCC
CCAGACGCTGGAGATCCTGGACATCACTCCCTGCAGCTTTGGGGGTGTCTCCGTCATCACGCCT
GGTACCAACACTTCCAACCAGGTGGCAGTGTTGTACCAGGACGTGAACTGCACGGAGGTGCCC
GTGGCAATCCACGCCGACCAGTTGACCCCAACCTGGCGCGTGTATTCCACAGGTAGCAACGTGT
TTCAGACCCGGGCCGGCTGTCTGATTGGCGCCGAGCACGTGAACAATAGCTACGAGTGCGACA
TCCCCATCGGGGCCGGAATCTGTGCGTCTTACCAGACGCAGACCAATTCCCCCCGGCGGGCTC
GCAGCGTAGCTAGCCAGTCAATCATAGCCTACACGATGTCCCTGGGCGCCGAGAACTCGGTGG
CCTATAGCAATAACTCTATTGCTATCCCTACTAACTTCACGATCTCGGTCACCACCGAGATCCTG
CCAGTTAGTATGACCAAGACCTCCGTGGATTGCACCATGTACATCTGTGGTGATTCCACGGAGT
GCAGCAACCTGCTGCTGCAGTACGGTTCATTCTGCACCCAGCTGAACCGTGCCCTGACAGGCAT
CGCCGTGGAGCAGGACAAGAACACCCAGGAGGTGTTTGCCCAGGTCAAGCAGATCTACAAGAC
ACCTCCTATCAAGGACTTCGGCGGATTCAACTTCTCCCAGATCCTCCCGGATCCGTCGAAGCCTT
CCAAGCGGTCCTTCATCGAGGACCTCCTGTTCAACAAGGTGACCCTGGCCGACGCCGGCTTCAT
CAAGCAGTACGGCGACTGCCTGGGGGACATCGCGGCCCGGGATCTGATTTGTGCCCAGAAGTT
CAACGGGCTGACTGTGCTTCCGCCTCTGCTGACCGACGAGATGATCGCTCAGTACACCTCCGCC
CTGCTGGCTGGGACCATCACCAGCGGGTGGACGTTCGGGGCTGGAGCCGCGCTCCAGATCCC
ATTCGCGATGCAGATGGCATACCGCTTCAACGGAATTGGAGTGACACAGAACGTGCTGTACGAG
AACCAGAAGCTGATCGCGAACCAGTTCAACTCCGCGATCGGCAAGATTCAGGACTCCCTGAGCT
CTACCGCCAGCGCCCTGGGCAAGCTGCAGGACGTGGTCAACCAGAACGCCCAGGCGCTGAACA
CCCTGGTGAAGCAGCTGTCCTCCAATTTCGGTGCCATCTCATCCGTGCTGAACGACATCTTGTCT
CGGCTGGACCCTCCCGAGGCGGAGGTGCAGATCGACCGCCTCATCACGGGGCGGCTGCAGTC
CCTGCAGACCTACGTGACTCAGCAGCTCATCAGGGCTGCTGAGATCAGGGCCAGCGCCAATCT
GGCAGCTACCAAGATGTCAGAGTGCGTGCTGGGCCAGAGCAAGAGGGTGGACTTCTGTGGCAA
GGGATATCACCTGATGTCCTTTCCACAGAGTGCACCCCATGGTGTGGTGTTCTTGCACGTGACC
TACGTGCCCGCCCAGGAGAAGAACTTCACAACCGCCCCAGCCATCTGCCACGACGGGAAGGCC
CACTTCCCTCGGGAGGGGGTCTTCGTCAGTAACGGTACTCACTGGTTCGTGACCCAGAGGAATT
TCTACGAGCCCCAGATCATCACCACTGACAATACCTTTGTGAGTGGGAACTGCGACGTCGTGAT
CGGTATTGTGAACAATACCGTGTACGACCCGCTGCAGCCTGAGCTGGATTCTTTCAAGGAGGAG
CTGGACAAGTACTTCAAGAATCACACGTCCCCCGACGTGGATCTGGGGGATATCTCTGGAATCA
ACGCCTCTGTGGTCAACATCCAGAAGGAGATCGACAGGCTCAACGAGGTGGCCAAGAACCTCAA
CGAGAGCCTGATCGATCTCCAGGAGCTCGGGAAGTACGAGCAGTACATCAAGTGGCCGTGGTA
CATCTGGCTGGGGTTCATTGCCGGACTCATCGCCATCGTGATGGTCACGATCATGCTGTGCTGC
ATGACTTCCTGCTGCAGCTGCCTCAAGGGCTGCTGCTCTTGCGGCAGCTGCTGCAAGTTCGACG
AGGACGACTCCGAGCCCGTCCTCAAGGGAGTCAAGCTGCACTACACCTAAATAAAAAAAAATTAA
AAAAAAAATTTAAAAAAAAATTAAAAAAATTTAAAAAAAATTAAAAAAAAAATAAAAAATTAAAAAAA
TAAAAAAAAAATTTAAAAAAAAATAAAAAAGCAACCCACACGACCGTTTAGACGCTACGGACTTAA
ATAATTGAGCCTTAGAGAAGAAATTCTTTAAGTGGATGCTCTCAAACTCAGGGAAACCTAAATCTA
GCTATAGACAAGGCAATCCTGAGCCAAGCCGAAGTAGTAATTAGTAAGTT.

### Nucleic Acid Sequences

In some embodiments, the antigenic polypeptide encoded by the polynucleotide is a corona virus, or a fragment or subunit thereof. In some embodiments, the antigenic polypeptide is spike protein (S) of a MERS virus (MERS-CoV), a SARS virus (SARS-CoV), or a fragment or subunit thereof.

In some embodiments, the antigenic polypeptide is a SARS virus, or a fragment or subunit thereof. The antigenic polypeptide may be a SARS-CoV-2 spike protein or a SARS-CoV-2 spike glycoprotein.

In some embodiments, the polynucleotide may be a mRNA, an siRNA or siRNA precursor, a microRNA (miRNA) or miRNA precursor, a plasmid, a Dicer substrate small interfering RNA (dsiRNA), a short hairpin RNA (shRNA), an asymmetric interfering RNA (aiRNA), a peptide nucleic acid (PNA), a morpholino, a locked nucleic acid (LNA), a piwi-interacting RNA (piRNA), a ribozyme, a deoxyribozyme (DNAzyme), an aptamer, a circular RNA (circRNA), a guide RNA (gRNA), or a DNA molecule encoding any of these RNAs. In one embodiment, the polynucleotide is an mRNA. In one embodiment, the polynucleotide is a circRNA.

In some embodiments, the polynucleotide encodes an infectious agent, disease, or disorder antigen variant (e.g., variant trimeric spike protein, such as a stabilized prefusion spike protein). Antigen variants or other polypeptide variants refer to molecules that differ in their amino acid sequence from a wild-type, native, or reference sequence. The antigen/polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native, or reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently, a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence that is soluble or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an RNA (e.g., mRNA or circRNA) vaccine.

As recognized by those skilled in the art, protein fragments, functional protein domains, and homologous proteins are also considered to be within the scope of antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the infectious agent. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full-length proteins.

In some embodiments, the antigenic polypeptide is a structural protein. In some embodiments, the antigenic polypeptide is a spike protein, an envelope protein, a nucleocapsid protein, or a membrane protein. In some embodiments, the antigenic polypeptide is a stabilized prefusion spike protein. In some embodiments, the RNA (e.g., mRNA or circRNA) comprises an open reading frame that encodes a variant trimeric spike protein. The trimeric spike protein, for example, may comprise a stabilized prefusion spike protein. In some embodiments, the stabilized prefusion spike protein a double proline (S2P) mutation.

In some embodiments, the polynucleotide (e.g., mRNA or circRNA) having an open reading frame (ORF) encoding an antigen (e.g., variant trimeric spike protein, such as a stabilized prefusion spike protein). In some embodiments, the RNA (e.g., mRNA or circRNA) further comprises a 5′ UTR, 3′ UTR, a poly(A) tail and/or a 5′ cap analog.

In some embodiments, the RNA (e.g., mRNA or circRNA) comprises a 5′ untranslated region (UTR) and/or a 3′ UTR.

Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA or circRNA), the “T”s would be substituted for “U”s. Thus, any of the DNAs disclosed and identified by a particular sequence identification number herein also disclose the corresponding RNA (e.g., mRNA or circRNA) sequence complementary to the DNA, where each “T” of the DNA sequence is substituted with “U”

In some embodiments, the RNA (e.g., mRNA or circRNA) is derived from (a) a DNA molecule; or (b) an RNA molecule. In the RNA (e.g., mRNA or circRNA), T is optionally substituted with U.

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. The sequences disclosed herein may further comprise additional elements, e.g., 5′ and 3′ UTRs, but that those elements, unlike the ORF, need not necessarily be present in a polynucleotide of the present disclosure.

### Stabilizing Elements

Naturally occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, the polynucleotide has an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Vims Capping Enzyme to generate the “Cap 0” structure: m7G(5′)ppp(5′)G (New England BioLabs, Ipswich, MA). Cap 1 structure may be generated using both Vaccinia Vims Capping Enzyme and a 2′-0 methyl-transferase to generate m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, the polynucleotide includes a stabilizing element. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the polynucleotide (e.g., mRNA) includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, b-Galactosidase, EGFP), or a marker or selection protein (e.g. alpha-Globin, Galactokinase and Xanthine:guanine phosphoribosyl transferase (GPT)).

In some embodiments, the polynucleotide (e.g., mRNA or circRNA) includes the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence. In some embodiments, an RNA (e.g., mRNA or circRNA) does not include a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

The polynucleotide (e.g., mRNA or circRNA) may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, the polynucleotide (e.g., mRNA or circRNA) has one or more AU-rich sequences removed. These sequences, sometimes referred to as AURES are destabilizing sequences found in the 3′UTR. The AURES may be removed from the RNA vaccines. Alternatively, the AURES may remain in the RNA vaccine.

### Signal Peptides

In some embodiments, the polynucleotide (e.g., mRNA or circRNA) has an ORF that encodes a signal peptide fused to the antigen. Signal peptides, comprising the N-terminal 15-60 amino acids of proteins, are typically needed for the translocation across the membrane on the secretory pathway and, thus, universally control the entry of most proteins both in eukaryotes and in prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane. A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than antigens in nature) are known in the art and can be tested for desired properties and then incorporated into a nucleic acid of the disclosure. In some embodiments, the signal peptide may comprise those described in WO 2021/154763, which is incorporated by reference in its entirety.

### Fusion Proteins

In some embodiments, the polynucleotide (e.g., mRNA or circRNA) encodes an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the antigen. Antigenic fusion proteins, in some embodiments, retain the functional property from each original protein.

### Scaffold Moieties

The polynucleotide (e.g., mRNA or circRNA), in some embodiments, encodes fusion proteins that comprise antigens linked to scaffold moieties. In some embodiments, such scaffold moieties impart desired properties to an antigen encoded by a nucleic acid of the disclosure. For example, scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of −22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300 A and 360 A diameter, corresponding to 180 or 240 protomers. In some embodiments, the coronavirus antigen is fused to HBsAG or HBcAG to facilitate self-assembly of nanoparticles displaying the coronavirus antigen.

In some embodiments, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Fawson D. M. et al. Nature. 1991; 349:541-544). Ferritin self-assembles into nanoparticles with robust thermal and chemical stability. Thus, the ferritin nanoparticle is well suited to carry and expose antigens.

Fumazine synthase (FS) is also well suited as a nanoparticle platform for antigen display. FS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The FS monomer is 150 amino acids long, and consists of beta-sheets along with tandem alpha-helices flanking its sides. A number of different quaternary structures have been reported for FS, illustrating its morphological versatility: from homopentamers up to symmetrical assemblies of 12 pentamers forming capsids of 150 A diameter. Even FS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

In some embodiments, the polynucleotide encodes a coronavirus antigen (e.g., SARS-CoV-2 S protein) fused to a foldon domain. The foldon domain may be, for example, obtained from bacteriophage T4 fibritin (see, e.g., Tao Y, et al. Structure. 1997 Jun. 15; 5(6):789-98).

### Linkers and Cleavable Peptides

In some embodiments, the polynucleotide (e.g., mRNA or circRNA) encodes more than one polypeptide, referred to herein as fusion proteins. In some embodiments, the polynucleotide (e.g., mRNA or circRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2 A peptides, has been described in the art (see for example, Kim, J. H. et al. (2011) PLoS ONE 6:el8556). In some embodiments, the linker is an F2A linker. In some embodiments, the fusion protein contains three domains with intervening linkers, having the structure: domain-linker-domain-linker-domain.

Cleavable linkers known in the art may be used in connection with the disclosure. Exemplary such linkers include F2A linkers, T2A linkers, P2A linkers, E2A linkers (See, e.g., WO2017/127750, which is incorporated herein by reference in its entirety). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA or circRNA encoding more than one antigen/polypeptide separately within the same molecule) may be suitable for use as provided herein.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a coronavirus antigen). In some embodiments, a codon optimized sequence shares less than 90% sequence identity to a naturally occurring or wild-type sequence (e.g., a naturally occurring or wild-type mRNA sequence encoding a coronavirus antigen). In some embodiments, a codon optimized sequence shares less than 85% sequence identity to a naturally occurring or wild-type sequence (e.g., a naturally occurring or wild-type mRNA sequence encoding a coronavirus antigen). In some embodiments, a codon optimized sequence shares less than 80% sequence identity to a naturally occurring or wild-type sequence (e.g., a naturally occurring or wild-type mRNA sequence encoding a coronavirus antigen). In some embodiments, a codon optimized sequence shares less than 75% sequence identity to a naturally occurring or wild-type sequence (e.g., a naturally occurring or wild-type mRNA sequence encoding a coronavirus antigen).

In some embodiments, a codon optimized sequence shares between 65% and 85% (e.g., between about 67% and about 85% or between about 67% and about 80%) sequence identity to a naturally occurring or wild-type sequence (e.g., a naturally occurring or wild-type mRNA sequence encoding a coronavirus antigen). In some embodiments, a codon optimized sequence shares between 65% and 75% or about 80% sequence identity to a naturally occurring or wild-type sequence (e.g., a naturally occurring or wild-type mRNA sequence encoding a coronavirus antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a coronavirus antigen encoded by a non-codon-optimized sequence. When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA or circRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA or circRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, the polynucleotide (e.g., mRNA or circRNA) is not chemically modified and comprises the standard ribonucleotides consisting of adenosine, guanosine, cytosine and uridine. In some embodiments, nucleotides and nucleosides of the polynucleotide (e.g., mRNA or circRNA) comprise standard nucleoside residues such as those present in transcribed RNA (e.g. A, G, C, or U). In some embodiments, nucleotides and nucleosides of the polynucleotide (e.g., mRNA or circRNA) comprise standard deoxyribonucleosides such as those present in DNA (e.g. dA, dG, dC, or dT).

### Chemical Modifications

The polynucleotide (e.g., mRNA or circRNA) comprises, in some embodiments, an RNA having an open reading frame encoding an antigen, wherein the nucleic acid comprises nucleotides and/or nucleosides that can be standard (unmodified) or modified as is known in the art. In some embodiments, nucleotides and nucleosides of the polynucleotide (e.g., mRNA or circRNA) comprise modified nucleotides or nucleosides. Such modified nucleotides and nucleosides can be naturally occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

The nucleic acids of the polynucleotide (e.g., mRNA or circRNA) can comprise standard nucleotides and nucleosides, naturally-occurring nucleotides and nucleosides, non-naturally-occurring nucleotides and nucleosides, or any combination thereof.

Nucleic acids of the polynucleotide (e.g., DNA nucleic acids and RNA nucleic acids, such as mRNA or circRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA or circRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA or circRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA or circRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA or circRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, the polynucleotide (e.g., mRNA or circRNA) comprises uridine at one or more or all uridine positions of the nucleic acid. In some embodiments, mRNAs or circRNAs are uniformly modified (e.g., fully modified, modified throughout the entire sequence) for a particular modification. For example, a nucleic acid can be uniformly modified with 1-methyl-pseudouridine, meaning that all uridine residues in the mRNA or circRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the polynucleotide (e.g., mRNA or circRNA) may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA or circRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C orA+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs or circRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Untranslated Regions (UTRs)

The polynucleotide (e.g., mRNA or circRNA) may comprise one or more regions or parts that act or function as an untranslated region. Where mRNAs or circRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′ UTR and 3′ UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′ UTRs have features that play roles in translation initiation. They harbor signatures like Kozak sequences, which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′ UTR also have been known to form secondary structures that are involved in elongation factor binding.

In some embodiments, the 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219, which are incorporated herein by reference in their entirety). CMV immediate-early 1 (IE1) gene (US2014/0206753, WO2013/185069, which are incorporated herein by reference in their entirety), the sequence GGGAUCCUACC (WO2014/144196) (SEQ ID NO: 31) may also be used. In another embodiment, 5′ UTR of a TOP gene is a 5′ UTR of a TOP gene lacking the 5′ TOP motif (the oligopyrimidine tract) (e.g., WO/2015/101414, WO2015/101415, WO/2015/062738, WO2015/024667, WO2015/024667; 5′ UTR element derived from ribosomal protein Large 32 (L32) gene (WO/2015/101414, WO2015/101415, WO/2015/062738), 5′ UTR element derived from the 5′ UTR of an hydroxysteroid (17-b) dehydrogenase 4 gene (HSD17B4) (WO2015/024667), or a 5′ UTR element derived from the 5′ UTR of ATP5A1 (WO2015/024667) can be used. In some embodiments, an internal ribosome entry site (IRES) is used instead of a 5′ UTR.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of the polynucleotide (e.g., mRNA or circRNA). When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

3′ UTRs may be heterologous or synthetic. With respect to 3′ UTRs, globin UTRs, including Xenopus b-globin UTRs and human b-globin UTRs are known in the art (U.S. Pat. Nos. 8,278,063, 9,012,219, US2011/0086907). A modified b-globin construct with enhanced stability in some cell types by cloning two sequential human b-globin 3′UTRs head to tail has been developed and is well known in the art (US2012/0195936, WO2014/071963). In addition, a2-globin, al-globin, UTRs and mutants thereof are also known in the art (WO2015/101415, WO2015/024667). Other 3′ UTRs described in the mRNA constructs in the non-patent literature include CYBA (Ferizi et al., 2015) and albumin (Thess et al., 2015). Other exemplary 3′ UTRs include that of bovine or human growth hormone (wild type or modified) (WO2013/185069, US2014/0206753, WO2014152774), rabbit b globin and hepatitis B virus (HBV), a-globin 3′ UTR and Viral VEEV 3′ UTR sequences are also known in the art. In some embodiments, the sequence UUUGAAUU (WO2014/144196) is used. In some embodiments, 3′ UTRs of human and mouse ribosomal protein are used. Other examples include rps93′UTR (WO2015/101414), FIG. 4 (WO2015/101415), and human albumin 7 (WO2015/101415).

Those of ordinary skill in the art will understand that 5′ UTRs that are heterologous or synthetic may be used with any desired 3′ UTR sequence. For example, a heterologous 5′ UTR may be used with a synthetic 3′ UTR with a heterologous 3′ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR, which may contain a strong Kozak translational initiation signal and/or a 3′ UTR, which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 2010/0293625 and PCT/US2014/069155, which are herein incorporated by reference in their entirety. It should be understood that any UTR from any gene may be incorporated into the regions of a nucleic acid. Furthermore, multiple wild-type UTRs of any known gene may be utilized. It is also within the scope of the present disclosure to provide artificial UTRs that are not variants of wild type regions. These UTRs or portions thereof may be placed in the same orientation as in the transcript from which they were selected or may be altered in orientation or location. Hence, a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 2010/0129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins that are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest that share at least one function, structure, feature, localization, origin, or expression pattern.

The untranslated region may also include translation enhancer elements (TEE). As a non-limiting example, the TEE may include those described in US Application No. 2009/0226470, herein incorporated by reference in its entirety, and those known in the art. In vitro Transcription of RNA cDNA encoding the polynucleotides described herein may be transcribed using an in vitro transcription (IVT) system. In vitro transcription of RNA is known in the art and is described in International Publication WO 2014/152027, which is incorporated by reference herein in its entirety. In some embodiments, the RNA of the present disclosure is prepared in accordance with any one or more of the methods described in WO 2018/053209 and WO 2019/036682, each of which is incorporated by reference herein.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to the infectious agent mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA, which is then isolated and purified. In some embodiments, the DNA template includes an RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA or circRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the polynucleotide (e.g., mRNA or circRNA) comprises 5′ terminal cap, for example, 7mG(5′)ppp(5′)NlmpNp.

### Chemical Synthesis

Solid-phase chemical synthesis. The polynucleotide (e.g., mRNA or circRNA) may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of the polynucleotide (e.g., mRNA or circRNA) by the sequential addition of monomer building blocks may be carried out in a liquid phase.

Combination of Synthetic Methods. The synthetic methods discussed above each have its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase-catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the polynucleotide (e.g., mRNA or circRNA) may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheo alveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC—HPLC), liquid chromatography-mass spectrometry (LCMS), capillary electrophoresis (CE) and capillary gel electrophoresis (CGE).

### Multivalent Vaccines

The LNMP/nucleic acid vaccines, as provided herein, may include RNA or multiple RNAs encoding two or more antigens of the same or different species. In some embodiments, the LNMP/nucleic acid vaccine includes an RNA or multiple RNAs encoding two or more coronavirus antigens. In some embodiments, the RNA may encode 1, 2, 3, 4, 5, 6, 7, 8, 9, 10, 11, 12, or more coronavirus antigens.

In some embodiments, two or more different RNA (e.g., mRNA or circRNA) encoding antigens may be formulated in the same lipid nanoparticle. In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately.

### Combination Vaccines

The LNMP/nucleic acid vaccines, as provided herein, may include an RNA or multiple RNAs encoding two or more antigens of the same or different viral strains. Also provided herein are combination vaccines that include RNA encoding one or more coronavirus and one or more antigen(s) of a different organism. Thus, the vaccines of the present disclosure may be combination vaccines that target one or more antigens of the same strain/species, or one or more antigens of different strains/species, e.g., antigens which induce immunity to organisms which are found in the same geographic areas where the risk of coronavirus infection is high or organisms to which an individual is likely to be exposed to when exposed to a coronavirus.

### Vaccine Administration

In some embodiments, the LNMP/nucleic acid vaccines can be administered to a subject (e.g., a mammalian subject, such as a human subject), and the RNA polynucleotides are translated in vivo to produce an antigenic polypeptide (antigen). An “effective amount” of a composition (e.g., comprising RNA) is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of the LNMP/nucleic acid vaccine provides an induced or boosted immune response as a function of antigen production in the cells of the subject. In some embodiments, an effective amount of the composition containing RNA polynucleotides having at least one chemical modifications are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the LNMP/nucleic acid vaccines may be used for treatment or prevention of a coronavirus infection. The LNMP/nucleic acid vaccines may be administered prophylactically or therapeutically as part of an active immunization scheme to healthy individuals or early in infection during the incubation phase or during active infection after onset of symptoms. In some embodiments, the amount of RNA provided to a cell, a tissue or a subject may be an amount effective for immune prophylaxis.

The LNMP/nucleic acid vaccines may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, the LNMP/nucleic acid vaccines may be administered intramuscularly, intranasally or intradermally, similarly to the administration of inactivated vaccines known in the art.

The LNMP/nucleic acid vaccines may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

In some embodiments, the nucleic acid vaccine induces germinal center formation. In some embodiments, the nucleic acid vaccine induces B cell class switching.

In some embodiments, the nucleic acid vaccine has lower systemic exposure. In certain embodiments, the vaccinated host exhibits lower liver expression of the encoded mRNA or circRNA. In some embodiments, the nucleic acid vaccine induces at least 2%, 5%, 10%, 15%, 20%, 25%, 30%, 35%, or 40% lower liver expression than a control lipid nanoparticle. In other embodiments, the vaccinated host maintains spleen expression. In some embodiments, the nucleic acid vaccine maintains spleen expression compared to a control lipid nanoparticle.

In some embodiments, the nucleic acid vaccine reduces detectable infectious particles in the nares compared to an unvaccinated control. In other embodiments, the nucleic acid vaccine reduces detectable infectious particles in the lungs compared to an unvaccinated control. In certain embodiments, the detectable infectious particles are reduced 2-fold, 3-fold, 5-fold, 10-fold, 20-fold, 30-fold, or more compared to an unvaccinated control.

### Kits

The present invention also provides a kit including a container having a LNMP composition described herein. The kit may further include instructional material for applying or delivering the LNMP composition to a target organism in accordance with a method of the present invention. The skilled artisan will appreciate that the instructions for applying the LNMP composition in the methods of the present invention can be any form of instruction. Such instructions include, but are not limited to, written instruction material (such as, a label, a booklet, a pamphlet), oral instructional material (such as on an audio cassette or CD) or video instructions (such as on a video tape or DVD).

The LNMP/nucleic acid vaccine may include material for a single administration (e.g., single dosage form), or may include material for multiple administrations (e.g., a “multidose” kit).

The informational material of the kits is not limited in its form. In one embodiment, the informational material may include information about production of the LNMP/nucleic acid vaccine described herein, the drug substance, or the drug product, molecular weight of the composition, the drug substance, or the drug product, concentration, date of expiration, batch or production site information, and so forth. In one embodiment, the informational material relates to methods for administering a dosage form of the LNMP/nucleic acid vaccine.

In addition to a dosage form of the LNMP/nucleic acid vaccine described herein, the kit may include other ingredients, such as a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, and/or a second agent for treating a condition or disorder described herein. Alternatively, the other ingredients may be included in the kit, but in different compositions or containers than a LNMP/nucleic acid vaccine described herein. In such embodiments, the kit may include instructions for admixing a LNMP/nucleic acid vaccine described herein and the other ingredients, or for using a LNMP/nucleic acid vaccine described herein together with the other ingredients.

In some embodiments, the components of the kit are stored under inert conditions (e.g., under Nitrogen or another inert gas such as Argon). In some embodiments, the components of the kit are stored under anhydrous conditions (e.g., with a desiccant). In some embodiments, the components are stored in a light blocking container such as an amber vial.

A dosage form of a LNMP/nucleic acid vaccine described herein may be provided in any form, e.g., liquid, dried or lyophilized form. It is preferred that a LNMP/nucleic acid vaccine described herein be substantially pure and/or sterile. When a LNMP/nucleic acid vaccine described herein is provided in a liquid solution, the liquid solution preferably is an aqueous solution, with a sterile aqueous solution being preferred. When a LNMP/nucleic acid vaccine described herein is provided as a dried form, reconstitution generally is by the addition of a suitable solvent. The solvent, e.g., sterile water or buffer, can optionally be provided in the kit.

The kit may include one or more containers for the LNMP/nucleic acid vaccine containing a dosage form described herein. In some embodiments, the kit contains separate containers, dividers or compartments for the composition and informational material. For example, the therapeutic composition or nucleic acid molecule may be contained in a bottle, vial, or syringe, and the informational material may be contained in a plastic sleeve or packet. In other embodiments, the separate elements of the kit are contained within a single, undivided container. For example, the dosage form of a LNMP/nucleic acid vaccine described herein is contained in a bottle, vial or syringe that has attached thereto the informational material in the form of a label. In some embodiments, the kit includes a plurality (e.g., a pack) of individual containers, each containing one or more unit dosage forms of a LNMP/nucleic acid vaccine described herein. For example, the kit includes a plurality of syringes, ampules, foil packets, or blister packs, each containing a single unit dose of a dosage form described herein.

The containers of the kits can be airtight, waterproof (e.g., impermeable to changes in moisture or evaporation), and/or light-tight.

The kit optionally includes a device suitable for use of the dosage form, e.g., a syringe, pipette, forceps, measured spoon, swab (e.g., a cotton swab or wooden swab), or any such device.

The kits of the invention may include dosage forms of varying strengths to provide a subject with doses suitable for one or more of the initiation phase regimens, induction phase regimens, or maintenance phase regimens described herein. Alternatively, the kit may include a scored tablet to allow the user to administered divided doses, as needed.

The invention may be further represented by the following embodiments.

Embodiment 1. A nucleic acid vaccine, comprising:
one or more polynucleotides encoding one or more antigenic polypeptides derived from an infectious agent that causes an infectious disease, disorder, or condition,
formulated within a lipid reconstructed natural messenger packs (LNMPs) comprising natural lipids and an ionizable lipid, wherein the ionizable lipid has two or more of the characteristics listed below:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heterorganic group separated by a chain of at least two atoms; and
(v) an N:P ratio of at least 3.
   Embodiment 2. A nucleic acid vaccine, comprising:
one or more polynucleotides encoding one or more antigenic polypeptides derived from an infectious agent that causes an infectious disease, disorder, or condition,
formulated within a lipid reconstructed natural messenger packs (LNMPs) comprising natural lipids and an ionizable lipid, wherein the ionizable lipid is selected from one of the following groups of compounds:
i) a compound of formula

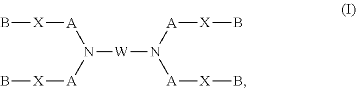

a pharmaceutically acceptable salt thereof, or a stereoisomer of any of the foregoing, wherein:
each A is independently C₁-C₁₆ branched or unbranched alkyl or C₁-C₁₆ branched or unbranched alkenyl, optionally substituted with heteroatom or substituted with OH, SH, or halogen;
each B is independently C₁-C₁₆ branched or unbranched alkyl or C₁-C₁₆ branched or unbranched alkenyl, optionally substituted with heteroatom or substituted with OH, SH, or halogen;
each X is independently a biodegradable moiety; and
W is

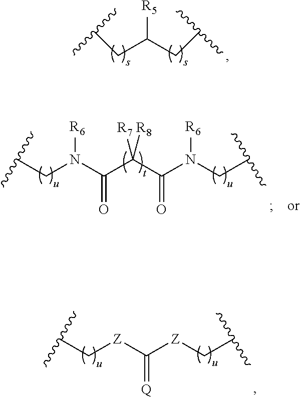

wherein:
   R₅ is OH, SH, or NR₁₀R₁₁;
   each R₆ is independently H, C₁-C₃ branched or unbranched alkyl, C₂-C₃ branched or unbranched alkenyl, or cycloalkyl;
   each R₇ and each R₈ is independently H, C₁-C₃ branched or unbranched alkyl, C₂-C₃ branched or unbranched alkenyl, halogen, OH, SH, or NR₁₀R₁₁, wherein each R₁₀ and R₁₁ is independently H, C₁-C₃ alkyl, or R₁₀ and R₁₁ are taken together to form a heterocyclic ring; R₇ and R^{a} are taken together to form a ring;
   each s is independently 1, 2, 3, 4, or 5;
   each u is independently 1, 2, 3, 4, or 5;
   t is 1, 2, 3, 4 or 5;
   each Z is independently absent, O, S, or NR₁₂, wherein R₁₂ is H, C₁-C₇ branched or unbranched alkyl, or C₂-C₇ branched or unbranched alkenyl, and
   Q is O, S, or NR₁₃, wherein each R₁₃ is H, or C₁-C₅ alkyl;
ii) a compound of formula

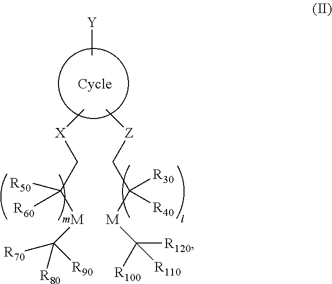

a pharmaceutically acceptable salt thereof, or a stereoisomer of any of the foregoing, wherein:

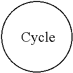

is a cyclic or heterocyclic moiety;
Y is alkyl, hydroxy, hydroxyalkyl or

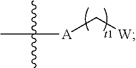

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each of X and Z is independently absent, —O—, —CO—, —N(R⁷)—, —O-alkylene-, -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, hydroxyalkyl, or aminoalkyl;
each M is independently a biodegradable moiety;
each of R₃₀, R₄₀, R₅₀, R₆₀, R₇₀, R₈₀, R₉₀, R₁₀₀, R₁₁₀, and R₁₂₀ is independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
each of l and m is an integer from 1 to 10;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl; and
iii) a compound of formula

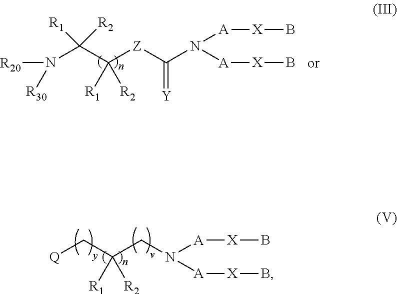

pharmaceutically acceptable salts thereof, and stereoisomers of any of the foregoing, wherein:
R₂₀ and R₃₀ are each independently H, C₁-C₅ branched or unbranched alkyl, or C₂-C₅ branched or unbranched alkenyl, or R₂₀ and R₃₀ together with the adjacent N atom form a 3 to 7 membered cyclic ring, optionally substituted with R^{a};
R^{a} is H, C₁-C₃ branched or unbranched alkyl, C₂-C₃ branched or unbranched alkenyl, halogen, OH, or SH;
each R₁ and each R₂ is independently H, C₁-C₃ branched or unbranched alkyl, C₂-C₃ branched or unbranched alkenyl, OH, halogen, SH, or NR₁₀R₁₁, or
R₁ and R₂ are taken together to form a cyclic ring;
each R₁₀ and R₁₁ is independently H, C₁-C₃ branched or unbranched alkyl, C₂-C₃ branched or unbranched alkenyl, or R₁₀ and R₁₁ are taken together to form a heterocyclic ring;
n is 0, 1, 2, 3 or 4;
Y is O or S;
Z is absent, O, S, or N(R₁₂), wherein each R₁₂ is independently H, C₁-C₇ branched or unbranched alkyl, or C₂-C₇ branched or unbranched alkenyl, provided that when Z is not absent, the adjacent R₁ and R₂ cannot be OH, NR₁₀R₁₁, or SH;
v is 0, 1, 2, 3, or 4;
y is 0, 1, 2, 3, or 4;
each A is each independently C₁-C₁₆ branched or unbranched alkyl, or C₂-C₁₆ branched or unbranched alkenyl, optionally interrupted with one or more heteroatoms or optionally substituted with OH, SH, or halogen;
each B is each independently C₁-C₁₆ branched or unbranched alkyl, or C₂-C₁₆ branched or unbranched alkenyl, optionally interrupted with one or more heteroatoms or optionally substituted with OH, SH, or halogen; and
each X is independently a biodegradable moiety; and
iv) a lipid comprising at least one head group and at least one tail group of formula (TI) or (TI′)

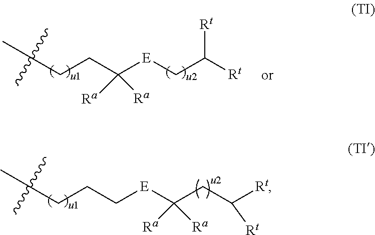

pharmaceutically acceptable salt thereof, or a stereoisomer of any of the foregoing,
wherein:
E is each independently —OC(O)—, —C(O)O—, —N(R⁷)C(O)—, —C(O)N(R⁷)—, —C(O—R₁₃)—O—, —C(O)O(CH₂)ᵣ—, —C(O)N(R⁷)(CH₂)ᵣ—, —S—S—, or —C(O—R₁₃)—O—(CH₂)ᵣ—, wherein each R⁷ is independently H, alkyl, alkenyl, cycloalkyl, hydroxyalkyl, or aminoalkyl;
R₁₃ is branched or unbranched C₃-C₁₀ alkyl;
r is 1, 2, 3, 4, or 5;
R^{a} is each independently C₁-C₅ alkyl, C₂-C₅ alkenyl, or C₂-C₅ alkynyl;
u1 and u2 are each independently 0, 1, 2, 3, 4, 5, 6, or 7;
R^{t} is each independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
[symbol] represents the bond connecting the tail group to the head group; and
wherein the lipid has a pKa from about 4 to about 8.
   Embodiment 3. The nucleic acid vaccine of Embodiment 2, wherein the ionizable lipid is a compound of group i), represented by a formula of

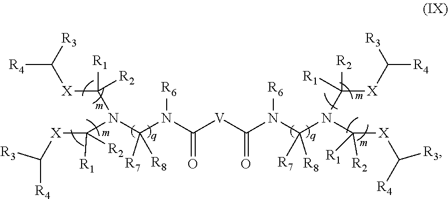

pharmaceutically acceptable salts thereof, and stereoisomers of any of the foregoing, wherein:
each R₁ and each R₂ is independently H, C₁-C₃ branched or unbranched alkyl, OH, halogen, SH, or NR₁₀R₁₁, or
each R₁ and each R₂ are independently taken together with the carbon atom(s) to which they are attached to form a cyclic ring;
each R₁₀ and R₁₁ is independently H, C₁-C₃ branched or unbranched alkyl, or R₁₀ and R₁₁ are taken together to form a heterocyclic ring;
each R₃ and each R₄ is independently H, C₂-C₁₄ branched or unbranched alkyl (e.g., C₃-C₁₀ branched or unbranched alkyl), or C₃-C₁₀ branched or unbranched alkenyl, provided that at least one of R₃ and R₄ is not H;
each X is independently a biodegradable moiety;
each q is independently 2, 3, 4, or 5;
V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups;
each R₆ is independently H, C₁-C₃ branched or unbranched alkyl, C₂-C₃ branched or unbranched alkenyl, or cycloalkyl;
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein each v is independently 0, 1, 2, 3, 4, or 5, and R₁₇ is OH, SH, or N(CH₃)₂; and
each m is independently 1, 2, 3, 4, 5, 6, 7, 8, 9 or 10.
   Embodiment 4. The nucleic acid vaccine of Embodiment 3, wherein V is a branched or unbranched C₂-C₃ alkylene, and each R₆ is independently H or methyl.
   Embodiment 5. The nucleic acid vaccine of Embodiment 2, wherein the ionizable lipid is a compound of group i), represented by a formula of

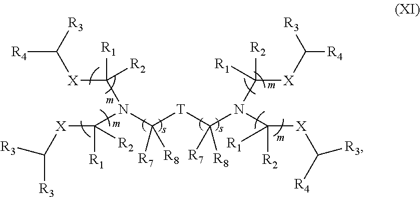

pharmaceutically acceptable salts thereof, and stereoisomers of any of the foregoing, wherein:
each R₁ and each R₂ is independently H, C₁-C₃ branched or unbranched alkyl, OH, halogen, SH, or NR₁₀R₁₁, or
each R₁ and each R₂ are independently taken together with the carbon atom(s) to which they are attached to form a cyclic ring;
each R₁₀ and R₁₁ is independently H, C₁-C₃ branched or unbranched alkyl, or R₁₀ and R₁₁ are taken together to form a heterocyclic ring;
each R₃ and each R₄ is independently H, C₂-C₁₄ branched or unbranched alkyl (e.g., C₃-C₁₀ branched or unbranched alkyl), or C₃-C₁₀ branched or unbranched alkenyl, provided that at least one of R₃ and R₄ is not H;
each X is independently a biodegradable moiety;
each s is independently 1, 2, 3, 4, or 5;
T is —NHC(O)O—, —OC(O)NH—, or a divalent heterocyclic optionally substituted with one or more —(CH₂)ᵥOH, —(CH₂)ᵥSH, or —(CH₂)ᵥ-halogen groups,
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein R₁₇ is OH, SH, or N(CH₃)₂;
each v is independently 0, 1, 2, 3, 4, or 5; and
each m is independently 1, 2, 3, 4, 5, 6, 7, 8, 9 or 10.
   Embodiment 6. The nucleic acid vaccine of Embodiment 5, wherein T is a divalent piperazine or a divalent dioxopiperazine.
   Embodiment 7. The nucleic acid vaccine of any one of Embodiments 3-6, wherein X is —OCO—, —COO—, —NHCO—, or —CONH—.
   Embodiment 8. The nucleic acid vaccine of Embodiment 2, wherein the ionizable lipid is a compound of group ii), represented by one of the following formulas:

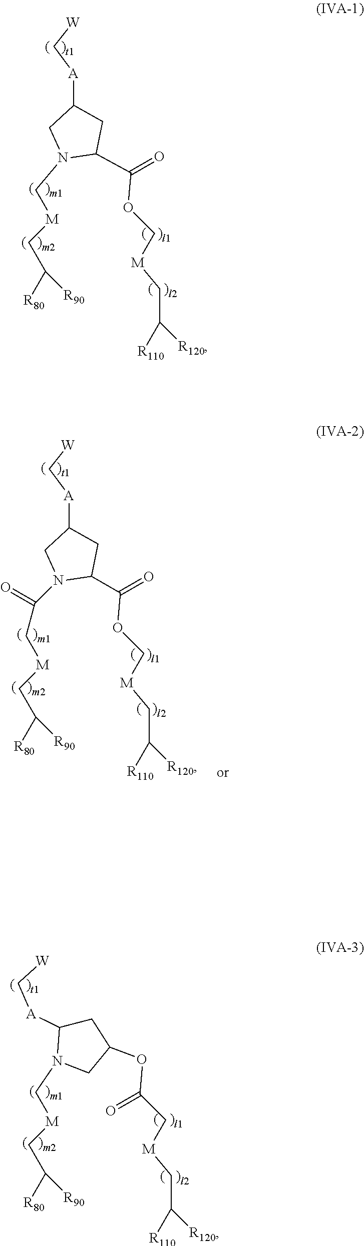

wherein:
A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each R⁷ is independently H, alkyl, alkenyl, cycloalkyl, hydroxy, hydroxyalkyl, or aminoalkyl;
t1 is an integer from 0 to 10;
W is hydroxyl, substituted or unsubstituted hydroxyalkyl, substituted or unsubstituted amino, substituted or unsubstituted aminocarbonyl, or substituted or unsubstituted heterocyclyl or heteroaryl;
each M is independently a biodegradable moiety;
each m1 is independently an integer from 3 to 6,
each l1 is independently an integer from 4 to 8,
m2 and l2 are each independently an integer from 0 to 3,
R₈₀ and R₉₀ are each independently unsubstituted C₅-C₈ alkyl or alkenyl; or R₈₀ is H or unsubstituted C₁-C₄ alkyl or alkenyl, and R₉₀ is unsubstituted C₅-C₁₁ alkyl or alkenyl; and
R₁₁₀ and R₁₂₀ are each independently unsubstituted C₅-C₈ alkyl or alkenyl; or R₁₁₀ is H or unsubstituted C₁-C₄ alkyl or alkenyl, and R₁₂₀ is unsubstituted C₅-C₁₁ alkyl or alkenyl.
   Embodiment 9. The nucleic acid vaccine of Embodiment 8, wherein:
M is —OC(O)— or —C(O)O—;

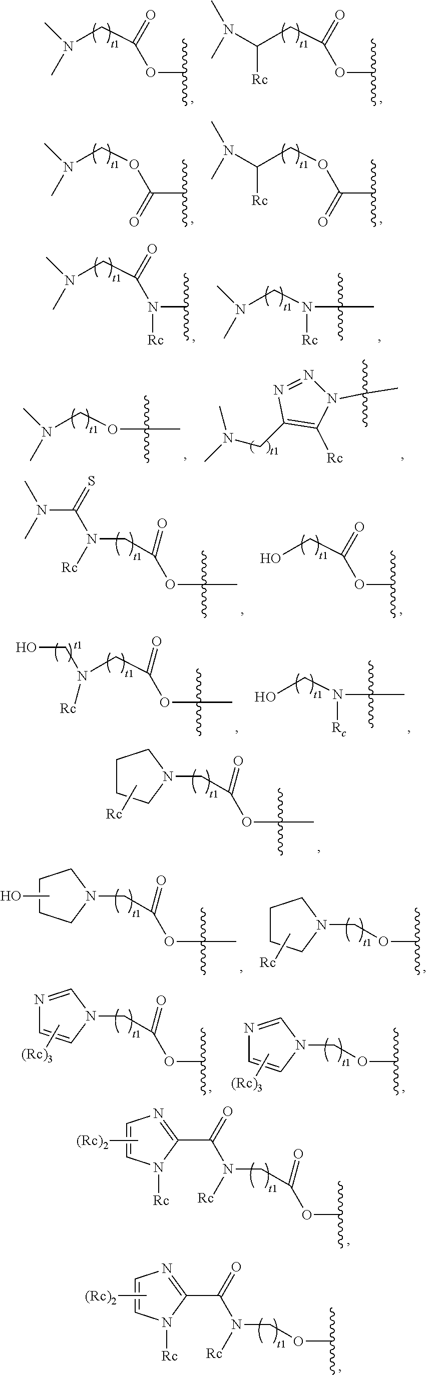

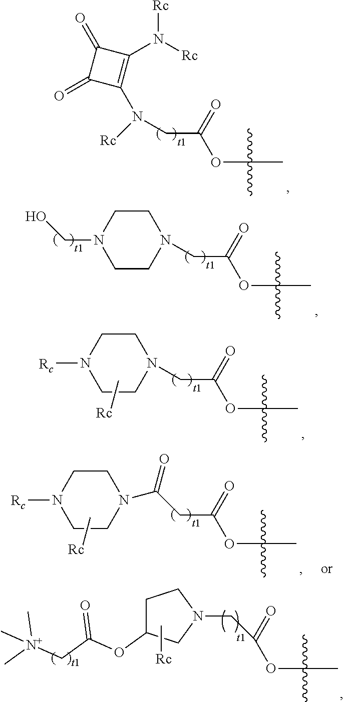

is: OH,

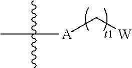

each R^{c} is independently H or C₁₋₃ alkyl; and
each t1 is independently 1, 2, 3, or 4.
   Embodiment 10. The nucleic acid vaccine of Embodiment 2, wherein the ionizable lipid is a compound of group iii), wherein R₁ and R₂ are each H, or each R₁ is H, and one of the R₂ variables is OH; and X is —OC(O)— or —C(O)O—.
   Embodiment 11. The nucleic acid vaccine of Embodiment 10, wherein the ionizable lipid is a compound of group iii), represented by formula III), wherein:
R₂₀ and R₃₀ are each independently H or C₁-C₃ branched or unbranched alkyl; or R₂₀ and R₃₀ together with the adjacent N atom form a 3 to 7 membered cyclic ring, optionally substituted with R^{a};
R^{a} is H or OH;
Z is absent, S, O, or NH; and
n is 0, 1, or 2.
   Embodiment 12. The nucleic acid vaccine of Embodiment 10, wherein the ionizable lipid is a compound of group iii), represented by formula V),
   Embodiment 13. The nucleic acid vaccine of Embodiment 2, wherein the ionizable lipid is a compound of group iv), comprising at least one head group and at least one tail group, wherein: the tail group has a structure of formula (TI) (or TI′); and
the head group has a structure of one of the following formulas:

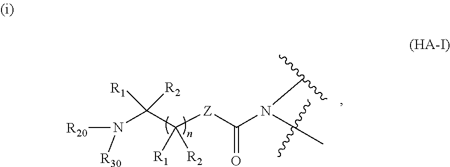

wherein:
   R₂₀ and R₃₀ are each independently H, C₁-C₅ branched or unbranched alkyl, or C₂-C₅ branched or unbranched alkenyl, optionally interrupted with one or more heteroatoms or substituted with OH, SH, halogen, or cycloalkyl groups; or
   R₂₀ and R₃₀, together with the adjacent N atom, form a 3 to 7 membered heterocyclic or heteroaromatic ring containing one or more heteroatoms, optionally substituted with one or more OH, SH, halogen, alkyl, or cycloalkyl groups;
   each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ together form a cyclic ring;
   each of R₁₀ and R₁₁ is independently H, C₁-C₃ branched or unbranched alkyl, C₂-C₃ branched or unbranched alkenyl; or R₁₀ and R₁₁ together form a heterocyclic ring;
   n is 0, 1, 2, 3 or 4; and
   Z is absent, O, S, or NR₁₂, wherein R₁₂ is H or C₁-C₇ branched or unbranched alkyl; provided that when Z is not absent, the adjacent R₁ and R₂ cannot be OH, NR₁₀R₁₁, or SH;

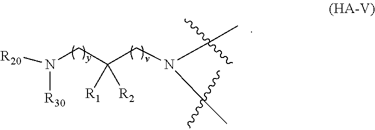

wherein:
   R₁ is H, C₁-C₃ alkyl, OH, halogen, SH, or NR₁₀R₁₁;
   R₂ is OH, halogen, SH, or NR₁₀R₁₁; or R₁ and R₂ can be taken together to form a cyclic ring;
   R₁₀ and R₁₁ are each independently H or C₁-C₃ alkyl; or R₁₀ and R₁₁ can be taken together to form a heterocyclic ring;
   R₂₀ and R₃₀ are each independently H, C₁-C₅ branched or unbranched alkyl, C₂-C₅ branched or unbranched alkenyl; or R₂₀ and R₃₀ can be taken together to form a cyclic ring; and
   each of v and y is independently 1, 2, 3, or 4;

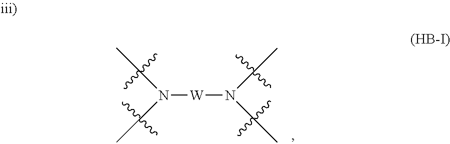

wherein W is

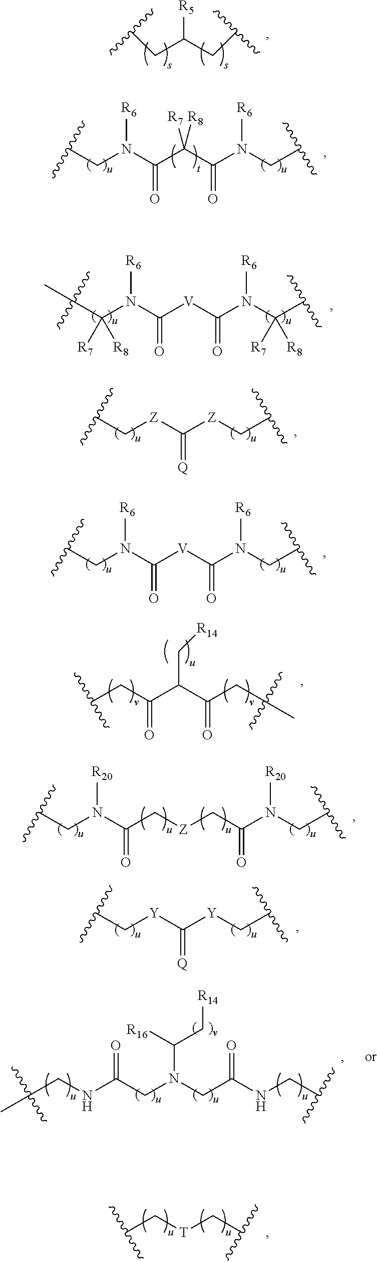

wherein
   R₅ is OH, SH, (CH₂)ₛOH, or NR₁₀R₁₁;
   each R₆ is independently H, C₁-C₃ branched or unbranched alkyl, C₂-C₃ branched or unbranched alkenyl, or cycloalkyl;
   each R₇ and R₈ are independently H, C₁-C₃ branched or unbranched alkyl, C₂-C₃ branched or unbranched alkenyl, halogen, (CH₂)ᵥOH, (CH₂)ᵥSH, (CH₂)ₛN(CH₃)₂, or NR₁₀R₁₁, wherein each R₁₀ and R₁₁ is independently H or C₁-C₃ alkyl, or R₁₀ and R₁₁ are taken together to form a heterocyclic ring; or R₇ and R₈ are taken together to form a ring;
   each R₂₀ is independently H, or C₁-C₃ branched or unbranched alkyl;
   R₁₄ is a heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H, C₁-C₃ alkyl, C₃-C₇ cycloalkyl, C₃-C₇ cycloalkenyl, optionally substituted with one or more NH and/or oxo groups, or R₁₀ and R₁₁ are taken together to form a heterocyclic ring;
   R₁₆ is H, ═O, ═S, or CN;
   each of s, u, and t is independently 1, 2, 3, 4, or 5;
   each v is independently 0, 1, 2, 3, 4, or 5;
   each Y is a divalent heterocyclic;
   each Z is independently absent, O, S, or NR₁₂, wherein R₁₂ is H, C₁-C₇ branched or unbranched alkyl, or C₂-C₇ branched or unbranched alkenyl;
   Q is O, S, CH₂, or NR₁₃, wherein each R₁₃ is H, or C₁-C₅ alkyl;
   V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups; and
   T is —NHC(O)O—, —OC(O)NH—, or a divalent heterocyclic; and
iv)

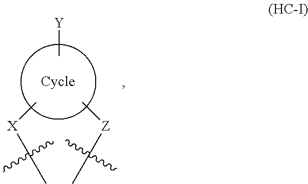

wherein:

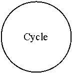

is cyclic or heterocyclic moiety;
   Y is alkyl, hydroxy, hydroxyalkyl,

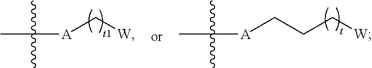

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, or —S—S—;
each of X and Z is independently absent, —O—, —C(O)—, —N(R⁷)—, —O-alkylene-, -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, alkoxy, hydroxyalkyl, alkylamino, alkylaminoalkyl, or aminoalkyl;
t is 0, 1, 2, or 3;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl; and
wherein the lipid has a pKa from about 4 to about 8.
   Embodiment 14. The nucleic acid vaccine of Embodiment 13, wherein the ionizable lipid is a compound of group iv), and wherein at least one tail group of the lipid has one of the following formulas:

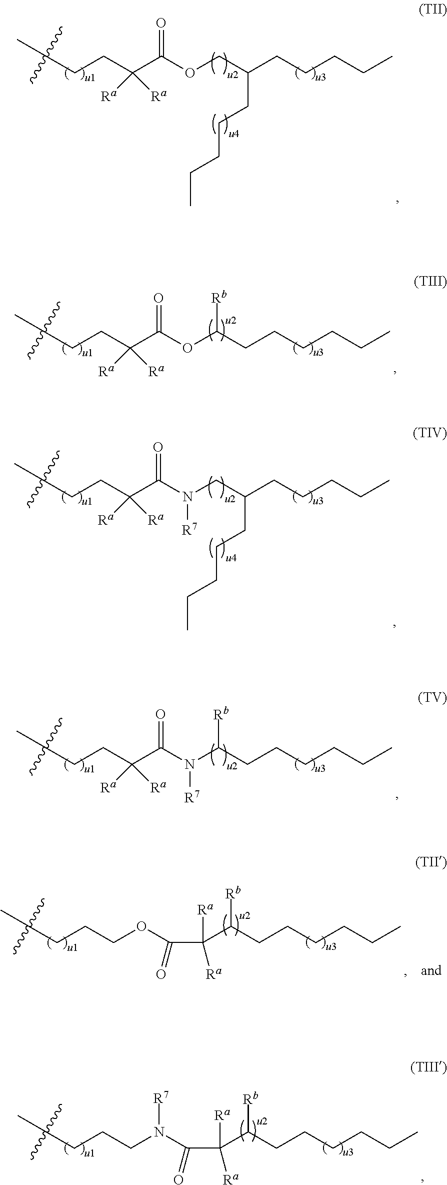

wherein:
R⁷ is each independently H or methyl;
R^{b} is in each occasion independently H or C₁-C₄ alkyl;
u1 and u2 are each independently 0, 1, 2, 3, 4, 5, 6, or 7; and
u3 and u4 are each independently 0, 1, 2, 3, 4, 5, 6, or 7; and
   the head group has a structure of one of the following formulas:

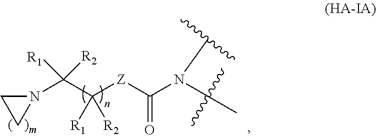

wherein m is 1, 2, 3, 4, 5, 6, 7 or 8;

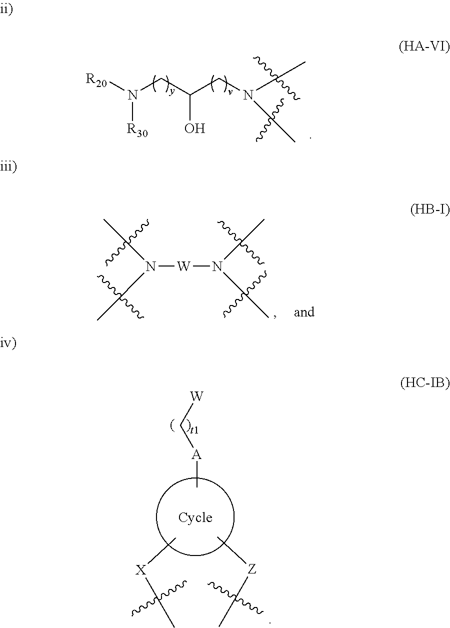

Embodiment 15. The nucleic acid vaccine of Embodiment 14, wherein at least one tail group has the structure of formula (TII), (TIII), (TIV), (TV), (TII′), and/or (TIII′), wherein u1 is 3-5, u2 is 0-3, u3 and u4 are each independently 1-7, and R^{a} is each independently methyl.
Embodiment 16. The nucleic acid vaccine of Embodiment 2, wherein the ionizable lipid is a compound in Table I, Table II, Table III, or Table IV.
Embodiment 17. The nucleic acid vaccine of Embodiment 16, wherein the ionizable lipid is

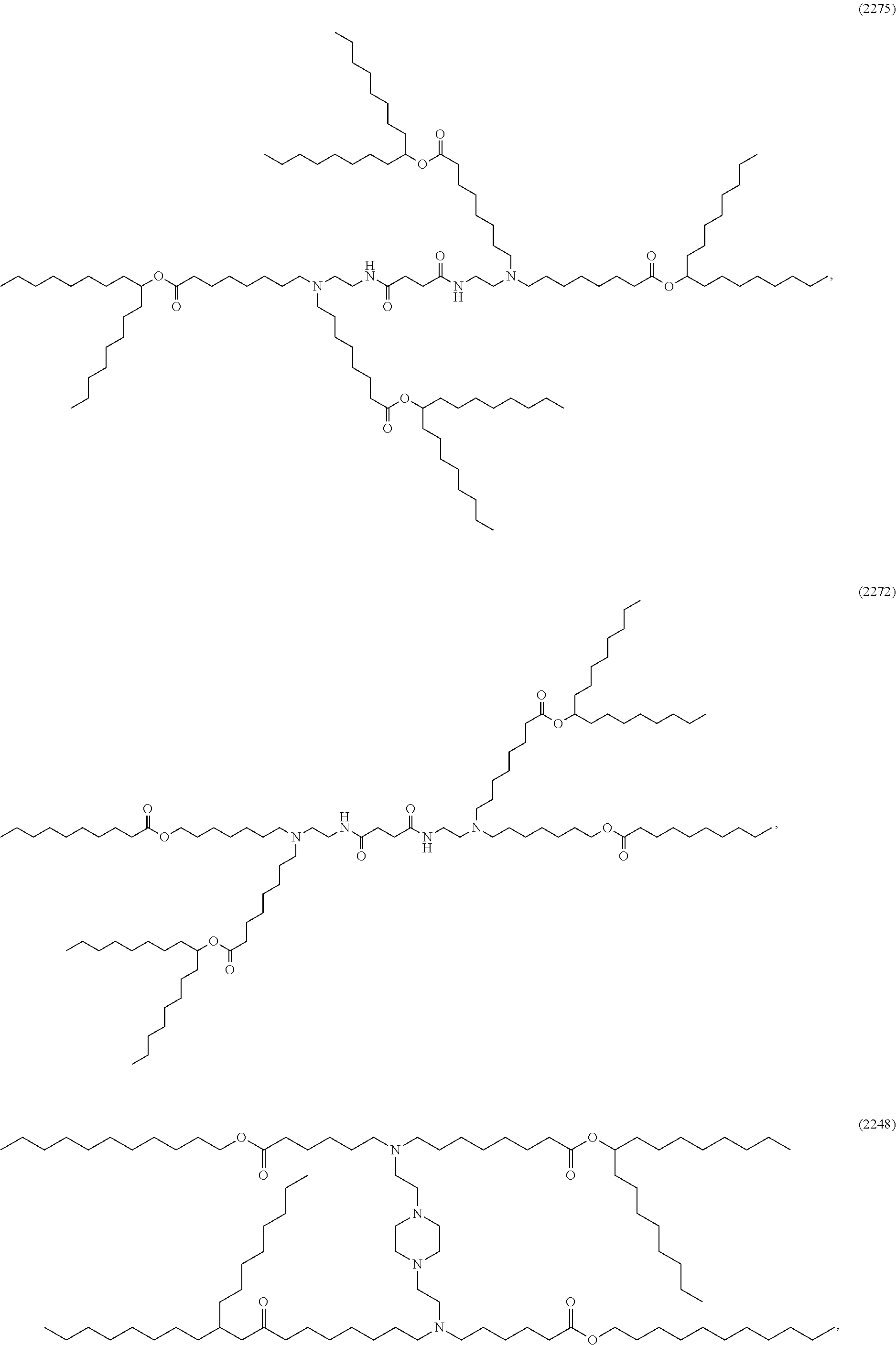

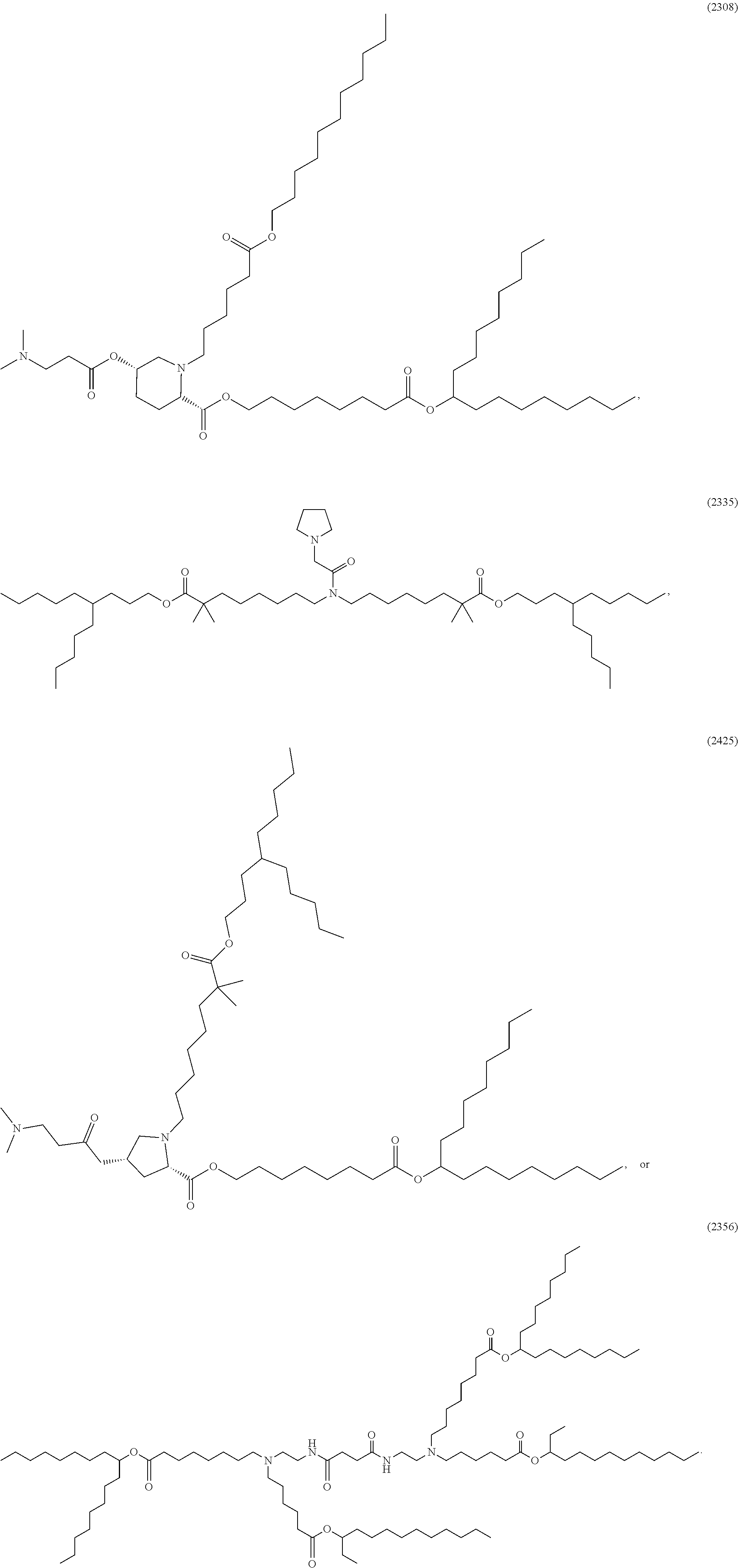

Embodiment 18. The nucleic acid vaccine of Embodiment 1 or 2, wherein the natural lipids are extracted from lemon or algae.
Embodiment 19. The nucleic acid vaccine of Embodiment 1 or 2, wherein the natural lipids are soy-derived lipids.
Embodiment 20. The nucleic acid vaccine of Embodiment 19, wherein the soy-derived lipids comprise soy PC, soy PE, soy PI, soy PA, lyso PC (soy LPC), lyso PI (soy LPI), soy PG, soyl PL (phospholipid) mixture, soy PS, soy LPS, soy polar, or a combination thereof.
Embodiment 21. The nucleic acid vaccine of Embodiment 1 or 2, wherein the natural lipids are extracted from E. coli or Salmonella typhimurium.
Embodiment 22. The nucleic acid vaccine of Embodiment 1 or 2, wherein the LNMPs further comprise a sterol and a polyethylene glycol (PEG)-lipid conjugate.
Embodiment 23. The nucleic acid vaccine of Embodiment 22, wherein the PEG-lipid conjugate is PEG-DMG or PEG-PE.
Embodiment 24. The nucleic acid vaccine of Embodiment 23, wherein the PEG-lipid conjugate is PEG-DMG and the PEG-DMG is PEG2000-DMG or PEG2000-PE.
Embodiment 25. The nucleic acid vaccine of Embodiment 22, wherein the LNMP comprises:
about 20 mol % to about 50 mol % of the ionizable lipid,
about 5 mol % to about 60 mol % of the natural lipids,
about 7 mol % to about 50 mol % of the sterol, and
about 0.5 mol % to about 3 mol % of the polyethylene glycol (PEG)-lipid conjugate.
   Embodiment 26. The nucleic acid vaccine of Embodiment 22, wherein the LNMPs comprise ionizable lipid:natural lipids:sterol:PEG-lipid at a molar ratio of about 35:50:12.5:2.5, about 35:20:42.5:2.5, about 50:20:28.5:1.5, about 45:20:33.5:2.5, or about 45:10:43.5:1.5.
   Embodiment 27. The nucleic acid vaccine of Embodiment 22, wherein the LNMPs further comprise a neutral lipid.
   Embodiment 28. The nucleic acid vaccine of Embodiment 27, wherein the neutral lipid is a phospholipid selected from the group consisting of lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof.
   Embodiment 29. The nucleic acid vaccine of Embodiment 27 or 28, wherein the LNMP comprises:
about 20 mol % to about 50 mol % of the ionizable lipid,
about 5 mol % to about 60 mol % of the natural lipids and the neutral lipid,
about 7 mol % to about 50 mol % of the sterol, and
about 0.5 mol % to about 3 mol % of the polyethylene glycol (PEG)-lipid conjugate.
   Embodiment 30. The nucleic acid vaccine of Embodiment 29, wherein the LNMPs comprise ionizable lipid:(natural lipids+ neutral lipid):sterol:PEG-lipid at a molar ratio of about 35:50:12.5:2.5.
   Embodiment 31. The nucleic acid vaccine of Embodiment 20, wherein the LNMPs comprise ionizable lipid:(natural lipids+ neutral lipid):sterol:PEG-lipid at a molar ratio of about 35:20:42.5:2.5.
   Embodiment 32. The nucleic acid vaccine of Embodiment 29, wherein the LNMPs comprise ionizable lipid:(natural lipids+ neutral lipid):sterol:PEG-lipid at a molar ratio of about 50:20:28.5:1.5.
   Embodiment 33. The nucleic acid vaccine of Embodiment 1 or 2, wherein the antigenic polypeptide is a corona virus, or a fragment or subunit thereof.
   Embodiment 34. The nucleic acid vaccine of Embodiment 33, wherein the antigenic polypeptide is spike protein (S) of a MERS virus (MERS-CoV), a SARS virus (SARS-CoV), or a fragment or subunit thereof.
   Embodiment 35. The nucleic acid vaccine of Embodiment 1 or 2, wherein the antigenic polypeptide is a SARS virus, or a fragment or subunit thereof.
   Embodiment 36. The nucleic acid vaccine of Embodiment 35, wherein the antigenic polypeptide is a SARS-CoV-2 spike protein or a SARS-CoV-2 spike glycoprotein.
   Embodiment 37. The nucleic acid vaccine of Embodiment 36, wherein the antigenic polypeptide is a wild-type SARS-CoV-2 spike glycoprotein.
   Embodiment 38. The nucleic acid vaccine of Embodiment 1 or 2, wherein the one or more polynucleotides comprise an mRNA or circRNA.
   Embodiment 39. The nucleic acid vaccine of Embodiment 38, wherein the mRNA or circRNA is derived from
(a) a DNA molecule; or
(b) an RNA molecule, wherein T is substituted with U.
   Embodiment 40. The nucleic acid vaccine of Embodiment 39, wherein the RNA molecule is a self-replicating RNA molecule.
   Embodiment 41. The nucleic acid vaccine of Embodiment 38, wherein the mRNA or circRNA comprises an open reading frame (ORF) that encodes a SARS-CoV-2 spike (S) glycoprotein having a double proline stabilizing mutation.
   Embodiment 42. The nucleic acid vaccine of Embodiment 41, wherein the double proline stabilizing mutation is at positions corresponding to K986 and V987 of a wild-type SARS-CoV-2 S glycoprotein.
   Embodiment 43. The nucleic acid vaccine of Embodiment 1 or 2, wherein the LNMP is a lipophilic moiety selected from the group consisting of a lipoplex, a liposome, a lipid nanoparticle, a polymer-based carrier, an exosome, a lamellar body, a micelle, and an emulsion.
   Embodiment 44. The nucleic acid vaccine of Embodiment 1 or 2, wherein the LNMP is a liposome selected from the group consisting of a cationic liposome, a nanoliposome, a proteoliposome, a unilamellar liposome, a multilamellar liposome, a ceramide-containing nanoliposome, and a multivesicular liposome.
   Embodiment 45. The nucleic acid vaccine of Embodiment 1 or 2, wherein the LNMP has a particle size of less than about 200 nm.
   Embodiment 46. The nucleic acid vaccine of Embodiment 45, wherein the LNMP has a particle size of less than about 100 nm.
   Embodiment 47. The nucleic acid vaccine of Embodiment 1 or 2, wherein the LNMP has an N:P ratio of 3 to 15.
   Embodiment 48. The nucleic acid vaccine of any one of the proceeding Embodiment s, wherein the nucleic acid vaccine has a total lipid:polynucleotide weight ratio of about 50:1 to about 10:1.
   Embodiment 49. The nucleic acid vaccine of Embodiment 48, wherein the nucleic acid vaccine has a total lipid:polynucleotide weight ratio of about 40:1 to about 28:1.
   Embodiment 50. The nucleic acid vaccine of Embodiment 48, wherein the nucleic acid vaccine has a total lipid:polynucleotide weight ratio of about 37:1 to about 33:1.
   Embodiment 51. The nucleic acid vaccine of Embodiment 1 or 2, wherein the infectious agent is a virus selected from the group consisting of a respiratory syncytial virus, a rhinovirus, an adenovirus, and a parainfluenza virus.
   Embodiment 52. The nucleic acid vaccine of embodiment 1 or 2, wherein the nucleic acid vaccine induces germinal center formation.
   Embodiment 53. The nucleic acid vaccine of embodiment 52, wherein the nucleic acid vaccine induces B cell class switching.
   Embodiment 54. The nucleic acid vaccine of embodiment 1 or 2, wherein the nucleic acid vaccine has at least 2%, 5%, 10%, 15%, 20%, 25%, 30%, 35%, or 40% lower liver expression than a control lipid nanoparticle without natural lipids.
   Embodiment 55. The nucleic acid vaccine of embodiment 1 or 2, wherein the nucleic acid vaccine induces a number of antigen-specific T cells in spleen cells at least comparable to, or 2%, 5%, 10%, 15%, 20%, 25%, 30%, 35%, 40%, 45%, 50%, 55%, 60%, 65%, 70%, 75%, 80%, 85%, 90%, 95%, or 100% higher than a control lipid nanoparticle without natural lipids.
   Embodiment 56. The nucleic acid vaccine of embodiment 1 or 2, wherein the nucleic acid vaccine reduces detectable infectious particles in the nares compared to an unvaccinated control.
   Embodiment 57. The nucleic acid vaccine of embodiment 1 or 2, wherein the nucleic acid vaccine reduces detectable infectious particles in the lungs compared to an unvaccinated control.
   Embodiment 58. The nucleic acid vaccine of embodiment 56 or 57, wherein the detectable infectious particles are reduced 2-fold, 3-fold, 5-fold, 10-fold, 20-fold, 30-fold, or more compared to an unvaccinated control.
   Embodiment 59. A method of preventing or reducing the transmission of an infectious disease, disorder, or condition, comprising:
administering to a subject the nucleic acid vaccine of Embodiment 1 or 2, thereby preventing or reducing the transmission of an infectious disease, disorder, or condition.
   Embodiment 60. The method of Embodiment 59, wherein the method prevents or reduces the transmission of the infectious agent from a vaccinated host to an unvaccinated host.
   Embodiment 61. The method of Embodiment 59, wherein the method prevents or reduces the transmission of the infectious agent from a vaccinated host to a vaccinated host.
   Embodiment 62. A nucleic acid vaccine, comprising:
one or more polynucleotides encoding one or more antigenic polypeptides derived from an infectious agent that causes an infectious disease, disorder, or condition,
formulated within a lipid nanoparticle (LNPs) comprising structural lipids and an ionizable lipid, wherein the ionizable lipid is selected from one of the following groups of compounds:
i) a compound of formula

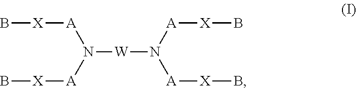

a pharmaceutically acceptable salt thereof, or a stereoisomer of any of the foregoing, wherein:
each A is independently C₁-C₁₆ branched or unbranched alkyl or C₁-C₁₆ branched or unbranched alkenyl, optionally substituted with heteroatom or substituted with OH, SH, or halogen;
each B is independently C₁-C₁₆ branched or unbranched alkyl or C₁-C₁₆ branched or unbranched alkenyl, optionally substituted with heteroatom or substituted with OH, SH, or halogen;
each X is independently a biodegradable moiety; and
W is

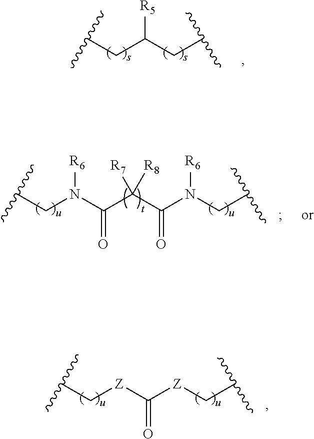

wherein:
   R₅ is OH, SH, NR₁₀R₁₁;
   each R₆ is independently H, C₁-C₃ branched or unbranched alkyl, C₂-C₃ branched or unbranched alkenyl, or cycloalkyl;
   each R₇ and each R₈ is independently H, C₁-C₃ branched or unbranched alkyl, C₂-C₃ branched or unbranched alkenyl, halogen, OH, SH, NR₁₀R₁₁, wherein each R₁₀ and R₁₁ is independently H, C₁-C₃ alkyl, or R₁₀ and R₁₁ are taken together to form a heterocyclic ring; R₇ and R₈ are taken together to form a ring;
   each s is independently 1, 2, 3, 4, or 5;
   each u is independently 1, 2, 3, 4, or 5;
   t is 1, 2, 3, 4 or 5;
   each Z is independently absent, O, S, or NR₁₂, wherein R₁₂ is H, C₁-C₇ branched or unbranched alkyl, or C₂-C₇ branched or unbranched alkenyl, and
   Q is O, S, or NR₁₃, wherein each R₁₃ is H, C₁-C₅ alkyl;
ii) a compound of formula

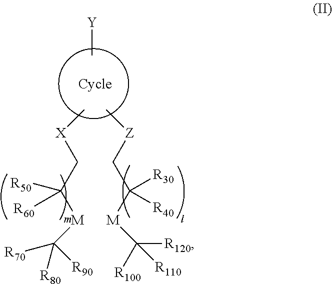

a pharmaceutically acceptable salt thereof, or a stereoisomer of any of the foregoing, wherein:

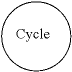

is a cyclic or heterocyclic moiety;
Y is alkyl, hydroxy, hydroxyalkyl or

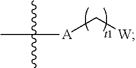

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each of X and Z is independently absent, —O—, —CO—, —N(R⁷)—, —O-alkylene-; -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, hydroxyalkyl, or aminoalkyl;
each M is independently a biodegradable moiety;
each of R₃₀, R₄₀, R₅₀, R₆₀, R₇₀, R₈₀, R₉₀, R₁₀₀, R₁₁₀, and R₁₂₀ is independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
each of l and m is an integer from 1 to 10;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl; and
iii) a compound of formula

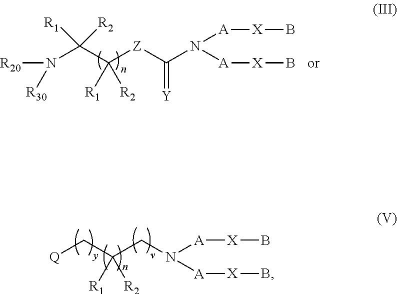

pharmaceutically acceptable salts thereof, and stereoisomers of any of the foregoing, wherein:
R₂₀ and R₃₀ are each independently H, C₁-C₅ branched or unbranched alkyl, or C₂-C₅ branched or unbranched alkenyl, or R₂₀ and R₃₀ together with the adjacent N atom form a 3 to 7 membered cyclic ring, optionally substituted with R^{a};
R^{a} is H, C₁-C₃ branched or unbranched alkyl, C₂-C₃ branched or unbranched alkenyl, halogen, OH, or SH;
each R₁ and each R₂ is independently H, C₁-C₃ branched or unbranched alkyl, C₂-C₃ branched or unbranched alkenyl, OH, halogen, SH, or NR₁₀R₁₁, or
R₁ and R₂ are taken together to form a cyclic ring;
each R₁₀ and R₁₁ is independently H, C₁-C₃ branched or unbranched alkyl, C₂-C₃ branched or unbranched alkenyl, or R₁₀ and R₁₁ are taken together to form a heterocyclic ring;
n is 0, 1, 2, 3 or 4;
Y is O or S;
Z is absent, O, S, or N(R₁₂)(R₁₂), wherein each R₁₂ is independently H, C₁-C₇ branched or unbranched alkyl, or C₂-C₇ branched or unbranched alkenyl, provided that when Z is not absent, the adjacent R₁ and R₂ cannot be OH, NR₁₀R₁₁, or SH;
u is 0, 1, 2, 3, 4, 5, 6, 7, or 8;
v is 0, 1, 2, 3, or 4;
y is 0, 1, 2, 3, or 4;
each A is each independently C₁-C₁₆ branched or unbranched alkyl, or C₂-C₁₆ branched or unbranched alkenyl, optionally interrupted with one or more heteroatoms or optionally substituted with OH, SH, or halogen;
each B is each independently C₁-C₁₆ branched or unbranched alkyl, or C₂-C₁₆ branched or unbranched alkenyl, optionally interrupted with one or more heteroatoms or optionally substituted with OH, SH, or halogen; and
each X is independently a biodegradable moiety; and
iv) a lipid comprising at least one head group and at least one tail group of formula (TI) or (TI′)

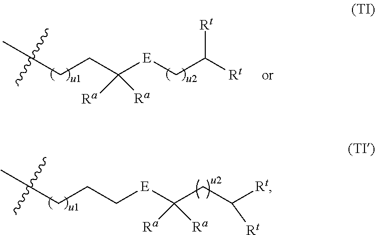

a pharmaceutically acceptable salt thereof, or a stereoisomer of any of the foregoing,
wherein:
E is each independently —OC(O)—, —C(O)O—, —N(R⁷)C(O)—, —C(O)N(R⁷)—, —C(O—R₁₃)—O—, —C(O)O(CH₂)ᵣ—, —C(O)N(R⁷)(CH₂)ᵣ—, —S—S—, or —C(O—R₁₃)—O—(CH₂)ᵣ—, wherein each R⁷ is independently H, alkyl, alkenyl, cycloalkyl, hydroxyalkyl, or aminoalkyl;
R₁₃ is branched or unbranched C₃-C₁₀ alkyl;
r is 1, 2, 3, 4, or 5;
R^{a} is each independently C₁-C₅ alkyl, C₂-C₅ alkenyl, or C₂-C₅ alkynyl;
u1 and u2 are each independently 0, 1, 2, 3, 4, 5, 6, or 7;
R^{t} is each independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
[symbol] represents the bond connecting the tail group to the head group; and wherein the lipid has a pKa from about 4 to about 8.

### EXAMPLES

The invention now being generally described, it will be more readily understood by reference to the following examples, which are included merely for purposes of illustration of certain aspects and embodiments of the present invention, and are not intended to limit the invention.

### Example 1: Preparation and Modification of LNMP, and Formulation LNMP with mRNAs

This example describes the preparation of a structural component comprising natural lipids (e.g., lipids extracted from a natural source), and the modification of the structural component to form a lipid reconstructed natural messenger pack (LNMP). An example of an LNMP is an LPMP. The preparation of natural messenger packs (NMPs), modification of NMP to prepare lipid reconstructed natural messenger packs (LNMP), and formulation of NMP and LNMP with mRNAs are illustrated by the preparation of PMP and preparation and formulation of LPMP, which may be accomplished utilizing the methods disclosed in International Patent Application Publication No. WO 2021/041301, which is incorporated herein by reference in its entirety.

In particular, all the experimental protocols disclosed in Examples 1-17 of International Patent Application Publication No. WO 2021/041301, are incorporated herein by reference in their entirety, including: Example 1. Isolation of Plant Messenger Packs from plants; Example 2. Production of purified Plant Messenger Packs (PMPs); Example 3. Plant Messenger Pack characterization; Example 4. Characterization of Plant Messenger Pack stability; Example 5. Loading PMPs with cargo; Example 6. Increasing PMP cellular uptake by formulation of PMPs with ionic liquids; Example 7. Modification of PMPs using ionizable lipids; Example 8. Formulation of LPMPs with microfluidics; Example 9. mRNA loading and delivery into lipid-reconstructed PMPs using ionizable lipids; Example 10. Cellular uptake of natural and reconstructed PMPs, with and without ionizable lipid modifications; Example 11. Increasing PMP cellular uptake by formulation of PMPs with cationic lipids; Example 12. Modification of PMPs using cationic lipids; Example 13. mRNA loading and delivery into lipid-reconstructed PMPs using cationic lipids; Example 14. Cellular uptake of natural and reconstructed PMPs, with and without cationic lipid modifications; Example 15. Improved loading using the cationic lipids GL67 and Ethyl PC; Example 16. Optimization of lipid ratios for mRNA loading; and Example 17. Optimization of lipid ratios for plasmid loading.

The preparation of NMP (and preparation and modification of LNMP to prepare LNMP) are also illustrated by the scenario where the natural lipids in the LNMP are extracted from a bacteria source. The extraction and isolation of a bacterial component comprising isolated bacterial extracellular vesicles (EVs) may be accomplished utilizing the methods disclosed in U.S. Patent Application Publication No. 2020/0254028, which is incorporated herein by reference in its entirety. The extraction and isolation of lipids from a bacterial source may be accomplished utilizing the methods disclosed in International Application Publication No. WO 2010/120939, International Application No. PCT/US22/50571, U.S. Pat. Nos. 7,847,113, and 8,592,188, which are incorporated herein by reference in their entirety.

Naturally or synthetically derived lipid may also be obtained from commercial sources. For instance, polar lipid extracts from E. coli can be obtained from Avanti Polar Lipids, Inc. (Alabaster, AL).

### Example 2: Preparation of Vaccine

### Manufacture and Characterization of Polynucleotides

The manufacture of polynucleotides and/or parts or regions thereof may be accomplished utilizing the methods taught in International Patent Application Publication No. WO 2014/152027, which is incorporated herein by reference in its entirety. Purification methods may include those taught in International Patent Application Publication Nos. WO2014/152030 and WO2014/152031, which are incorporated herein by reference in their entirety. Detection and characterization methods of the polynucleotides may be performed as taught in International Patent Application Publication No. WO2014/144039, which is incorporated herein by reference in its entirety. Characterization of the polynucleotides may be accomplished using polynucleotide mapping, reverse transcriptase sequencing, charge distribution analysis, detection of RNA impurities, or any combination of two or more of the foregoing. “Characterizing” comprises determining the RNA transcript sequence, determining the purity of the RNA transcript, or determining the charge heterogeneity of the RNA transcript, for example. Such methods are taught in, for example, International Patent Application Publication Nos. WO2014/144711 and WO2014/144767, which are incorporated herein by reference in their entirety.

Additional details of the mRNA design and modifications may be found in WO 2021/154763 and WO 2021/188969, which are incorporated herein by reference in their entirety.

Formulation of LNMP and LNP with SARS-CoV-2 S mRNA

### General Protocols and Experimental Designs

The protocols and detailed experimental procedures of the preparation of natural messenger packs (NMP), and modification of NMP to prepare lipid reconstructed natural messenger packs (LNMP), and formulations of NMP and LNMP with mRNAs have been discussed in Example 1, which lists all the experimental protocols disclosed in Examples 1-17 of International Patent Application Publication No. WO 2021/041301, all of which are incorporated herein by reference in their entirety. Additionally, the protocols and detailed experimental procedures of the preparation and formulations of LNMP (with or without mRNA) where the natural lipids in the LNMP are extracted from a bacteria source have been discussed in International Application No. PCT/US22/50571, which is incorporated herein by reference in its entirety. These protocols and detailed experimental procedures were followed when preparing NMP (e.g., with bacteria lipids), LNMP (e.g., with bacteria lipids), PMP, LPMP, LNP, and NMP (e.g., with bacteria lipids), LNMP (e.g., with bacteria lipids), PMP, LPMP, or LNP formulated with mRNAs in this example.

Briefly, the isolation and purification of crude plant messenger packs (PMPs) from lemon and algae, and characterization of these PMPs followed the experimental designs and protocols for plants sources described in Example 1. Isolation of Plant Messenger Packs from plants; Example 2. Production of purified Plant Messenger Packs (PMPs); Example 3. Plant Messenger Pack characterization; and Example 4. Characterization of Plant Messenger Pack stability, International Patent Application Publication No. WO 2021/041301, which is incorporated herein by reference in its entirety.

The modifications of natural PMP or reconstructed lemon LPMPs with cholesterol and PEG-lipid followed the experimental designs and protocols for plants sources described in Example 6. Increasing PMP cellular uptake by formulation of PMPs with ionic liquids; Example 7. Modification of PMPs using ionizable lipids; Example 10. Cellular uptake of natural and reconstructed PMPs, with and without ionizable lipid modifications; Example 11. Increasing PMP cellular uptake by formulation of PMPs with cationic lipids; Example 12. Modification of PMPs using cationic lipids; and Example 14. Cellular uptake of natural and reconstructed PMPs, with and without cationic lipid modifications, International Patent Application Publication No. WO 2021/041301, which is incorporated herein by reference in its entirety.

Formulations of the PMPs and lemon-lipid- or bacterial-lipid-reconstructed LPMPs with mRNAs followed the experimental designs and protocols for nucleic acids loading described in Example 5. Loading PMPs with cargo; Example 8. Formulation of LPMPs with microfluidics; Example 9. mRNA loading and delivery into lipid-reconstructed PMPs using ionizable lipids; Example 13. mRNA loading and delivery into lipid-reconstructed PMPs using cationic lipids; Example 15. Improved loading using the cationic lipids GL67 and Ethyl PC; Example 16. Optimization of lipid ratios for mRNA loading; and Example 17. Optimization of lipid ratios for plasmid loading, International Patent Application Publication No. WO 2021/041301, which is incorporated herein by reference in its entirety.

The preparation and formulations of LNMP (with or without mRNA) where the natural lipids in the LNMP is extracted from a bacteria source followed the experimental designs and protocols for bacterial derived composition described in Examples 1-5 in International Application No. PCT/US22/50571, which is incorporated herein by reference in its entirety.

### Example 3: Testing of LNMP/SARS-CoV-2 Formulations in Mice

If not specified, the LNP/mRNA or LNMP/mRNA formulations were prepared according to those described in Example 1-2. The experiment was designed to test the immunogenicity in mice of the candidate coronavirus vaccines comprising an mRNA of Table 1 encoding a coronavirus antigen (e.g., the spike (S) protein), such as a SARS-CoV-2 antigen.

TABLE 1
SARS-COV-2 spike (S) mRNA sequence (with modified cap)

| Element | Description | Position |
|---|---|---|
| cap | A modified 5′-cap1 structure (m⁷G+m³′-5′-ppp-5′-Am) | 1-2 |
| 5′-UTR | 5′-untranslated region derived from human alpha-globin RNA | 3-54 |
| | with an optimized Kozak sequence | |
| sig | S glycoprotein signal peptide (extended leader sequence), | 55-102 |
| | which guides translocation of the nascent polypeptide chain into | |
| | the endoplasmic reticulum. | |
| S protein_mut | Codon-optimized sequence encoding full-length SARS-COV-2 | 103-3879 |
| | spike (S) glycoprotein containing mutations K986P and V987P | |
| | to ensure the S glycoprotein remains in an antigenically optimal | |
| | pre-fusion conformation; stop codons: 3874-3879 (underlined) | |
| 3′-UTR | The 3′ untranslated region comprises two sequence elements | 3880-4174 |
| | derived from the amino-terminal enhancer of split (AES) mRNA | |
| | and the mitochondrial encoded 12S ribosomal RNA to confer | |
| | RNA stability and high total protein expression. | |
| poly(A) | A 110-nucleotide poly(A)-tail consisting of a stretch of 30 | 4175-4284 |
| | adenosine residues, followed by a 10-nucleotide linker | |
| | sequence and another 70 adenosine residues. | |

An example of the coding portion of the delivered cargo S protein mRNA is as follows:

(SEQ ID NO: 28)
uaauacgacucacuauaagGAGAAUAAACUAGUAUUCUUCUGGUCCCCACAGACUCAGAGAGAACCCG
CCACCAUGUUCGUGUUCCUGGUGCUGCUGCCUCUGGUGUCCAGCCAGUGUGUGAACCUGACC
ACCAGAACACAGCUGCCUCCAGCCUACACCAACAGCUUUACCAGAGGCGUGUACUACCCCGACA
AGGUGUUCAGAUCCAGCGUGCUGCACUCUACCCAGGACCUGUUCCUGCCUUUCUUCAGCAACG
UGACCUGGUUCCACGCCAUCCACGUGUCCGGCACCAAUGGCACCAAGAGAUUCGACAACCCCG
UGCUGCCCUUCAACGACGGGGUGUACUUUGCCAGCACCGAGAAGUCCAACAUCAUCAGAGGCU
GGAUCUUCGGCACCACACUGGACAGCAAGACCCAGAGCCUGCUGAUCGUGAACAACGCCACCA
ACGUGGUCAUCAAAGUGUGCGAGUUCCAGUUCUGCAACGACCCCUUCCUGGGCGUCUACUACC
ACAAGAACAACAAGAGCUGGAUGGAAAGCGAGUUCCGGGUGUACAGCAGCGCCAACAACUGCA
CCUUCGAGUACGUGUCCCAGCCUUUCCUGAUGGACCUGGAAGGCAAGCAGGGCAACUUCAAGA
ACCUGCGCGAGUUCGUGUUUAAGAACAUCGACGGCUACUUCAAGAUCUACAGCAAGCACACCC
CUAUCAACCUCGUGCGGGAUCUGCCUCAGGGCUUCUCUGCUCUGGAACCCCUGGUGGAUCUG
CCCAUCGGCAUCAACAUCACCCGGUUUCAGACACUGCUGGCCCUGCACAGAAGCUACCUGACA
CCUGGCGAUAGCAGCAGCGGAUGGACAGCUGGUGCCGCCGCUUACUAUGUGGGCUACCUGCA
GCCUAGAACCUUCCUGCUGAAGUACAACGAGAACGGCACCAUCACCGACGCCGUGGAUUGUGC
UCUGGAUCCUCUGAGCGAGACAAAGUGCACCCUGAAGUCCUUCACCGUGGAAAAGGGCAUCUA
CCAGACCAGCAACUUCCGGGUGCAGCCCACCGAAUCCAUCGUGCGGUUCCCCAAUAUCACCAA
UCUGUGCCCCUUCGGCGAGGUGUUCAAUGCCACCAGAUUCGCCUCUGUGUACGCCUGGAACC
GGAAGCGGAUCAGCAAUUGCGUGGCCGACUACUCCGUGCUGUACAACUCCGCCAGCUUCAGCA
CCUUCAAGUGCUACGGCGUGUCCCCUACCAAGCUGAACGACCUGUGCUUCACAAACGUGUACG
CCGACAGCUUCGUGAUCCGGGGAGAUGAAGUGCGGCAGAUUGCCCCUGGACAGACAGGCAAG
AUCGCCGACUACAACUACAAGCUGCCCGACGACUUCACCGGCUGUGUGAUUGCCUGGAACAGC
AACAACCUGGACUCCAAAGUCGGCGGCAACUACAAUUACCUGUACCGGCUGUUCCGGAAGUCC
AAUCUGAAGCCCUUCGAGCGGGACAUCUCCACCGAGAUCUAUCAGGCCGGCAGCACCCCUUGU
AACGGCGUGGAAGGCUUCAACUGCUACUUCCCACUGCAGUCCUACGGCUUUCAGCCCACAAAU
GGCGUGGGCUAUCAGCCCUACAGAGUGGUGGUGCUGAGCUUCGAACUGCUGCAUGCCCCUGC
CACAGUGUGCGGCCCUAAGAAAAGCACCAAUCUCGUGAAGAACAAAUGCGUGAACUUCAACUUC
AACGGCCUGACCGGCACCGGCGUGCUGACAGAGAGCAACAAGAAGUUCCUGCCAUUCCAGCAG
UUUGGCCGGGAUAUCGCCGAUACCACAGACGCCGUUAGAGAUCCCCAGACACUGGAAAUCCUG
GACAUCACCCCUUGCAGCUUCGGCGGAGUGUCUGUGAUCACCCCUGGCACCAACACCAGCAAU
CAGGUGGCAGUGCUGUACCAGGACGUGAACUGUACCGAAGUGCCCGUGGCCAUUCACGCCGA
UCAGCUGACACCUACAUGGCGGGUGUACUCCACCGGCAGCAAUGUGUUUCAGACCAGAGCCGG
CUGUCUGAUCGGAGCCGAGCACGUGAACAAUAGCUACGAGUGCGACAUCCCCAUCGGCGCUG
GAAUCUGCGCCAGCUACCAGACACAGACAAACAGCCCUCGGAGAGCCAGAAGCGUGGCCAGCC
AGAGCAUCAUUGCCUACACAAUGUCUCUGGGCGCCGAGAACAGCGUGGCCUACUCCAACAACU
CUAUCGCUAUCCCCACCAACUUCACCAUCAGCGUGACCACAGAGAUCCUGCCUGUGUCCAUGA
CCAAGACCAGCGUGGACUGCACCAUGUACAUCUGCGGCGAUUCCACCGAGUGCUCCAACCUGC
UGCUGCAGUACGGCAGCUUCUGCACCCAGCUGAAUAGAGCCCUGACAGGGAUCGCCGUGGAA
CAGGACAAGAACACCCAAGAGGUGUUCGCCCAAGUGAAGCAGAUCUACAAGACCCCUCCUAUCA
AGGACUUCGGCGGCUUCAAUUUCAGCCAGAUUCUGCCCGAUCCUAGCAAGCCCAGCAAGCGGA
GCUUCAUCGAGGACCUGCUGUUCAACAAAGUGACACUGGCCGACGCCGGCUUCAUCAAGCAGU
AUGGCGAUUGUCUGGGCGACAUUGCCGCCAGGGAUCUGAUUUGCGCCCAGAAGUUUAACGGA
CUGACAGUGCUGCCUCCUCUGCUGACCGAUGAGAUGAUCGCCCAGUACACAUCUGCCCUGCUG
GCCGGCACAAUCACAAGCGGCUGGACAUUUGGAGCAGGCGCCGCUCUGCAGAUCCCCUUUGC
UAUGCAGAUGGCCUACCGGUUCAACGGCAUCGGAGUGACCCAGAAUGUGCUGUACGAGAACCA
GAAGCUGAUCGCCAACCAGUUCAACAGCGCCAUCGGCAAGAUCCAGGACAGCCUGAGCAGCAC
AGCAAGCGCCCUGGGAAAGCUGCAGGACGUGGUCAACCAGAAUGCCCAGGCACUGAACACCCU
GGUCAAGCAGCUGUCCUCCAACUUCGGCGCCAUCAGCUCUGUGCUGAACGAUAUCCUGAGCAG
ACUGGACCCUCCUGAGGCCGAGGUGCAGAUCGACAGACUGAUCACAGGCAGACUGCAGAGCCU
CCAGACAUACGUGACCCAGCAGCUGAUCAGAGCCGCCGAGAUUAGAGCCUCUGCCAAUCUGGC
CGCCACCAAGAUGUCUGAGUGUGUGCUGGGCCAGAGCAAGAGAGUGGACUUUUGCGGCAAGG
GCUACCACCUGAUGAGCUUCCCUCAGUCUGCCCCUCACGGCGUGGUGUUUCUGCACGUGACA
UAUGUGCCCGCUCAAGAGAAGAAUUUCACCACCGCUCCAGCCAUCUGCCACGACGGCAAAGCC
CACUUUCCUAGAGAAGGCGUGUUCGUGUCCAACGGCACCCAUUGGUUCGUGACACAGCGGAAC
UUCUACGAGCCCCAGAUCAUCACCACCGACAACACCUUCGUGUCUGGCAACUGCGACGUCGUG
AUCGGCAUUGUGAACAAUACCGUGUACGACCCUCUGCAGCCCGAGCUGGACAGCUUCAAAGAG
GAACUGGACAAGUACUUUAAGAACCACACAAGCCCCGACGUGGACCUGGGCGAUAUCAGCGGA
AUCAAUGCCAGCGUCGUGAACAUCCAGAAAGAGAUCGACCGGCUGAACGAGGUGGCCAAGAAU
CUGAACGAGAGCCUGAUCGACCUGCAAGAACUGGGGAAGUACGAGCAGUACAUCAAGUGGCCC
UGGUACAUCUGGCUGGGCUUUAUCGCCGGACUGAUUGCCAUCGUGAUGGUCACAAUCAUGCU
GUGUUGCAUGACCAGCUGCUGUAGCUGCCUGAAGGGCUGUUGUAGCUGUGGCAGCUGCUGCA
AGUUCGACGAGGACGAUUCUGAGCCCGUGCUGAAGGGCGUGAAACUGCACUACACAUGAUGAC
UCGAGCUGGUACUGCAUGCACGCAAUGCUAGCUGCCCCUUUCCCGUCCUGGGUACCCCGAGU
CUCCCCCGACCUCGGGUCCCAGGUAUGCUCCCACCUCCACCUGCCCCACUCACCACCUCUGCU
AGUUCCAGACACCUCCCAAGCACGCAGCAAUGCAGCUCAAAACGCUUAGCCUAGCCACACCCCC
ACGGGAAACAGCAGUGAUUAACCUUUAGCAAUAAACGAAAGUUUAACUAAGCUAUACUAACCCC
AGGGUUGGUCAAUUUCGUGCCAGCCACACCCUGGAGCUAGCAAAAAAAAAAAAAAAAAAAAAAA
AAAAAAAGCAUAUGACUAAAAAAAAAAAAAAAAAAAAAAAAAAAAAAAAAAAAAAAAAAAAAAAAAA
AAAAAAAAAAAAAAAAAAAAcggcu

### Formulation and Characterization of LNMP/mRNA

This example further describes the formulation of several LNMPs formulated with ionizable lipids, structural lipids (natural or natural combined with synthetic), sterols, and PEG lipids, to encapsulate mRNA (e.g., S mRNA) for LNMP/mRNA. LNMP compositions where the natural lipids were extracted from a plant source, i.e., lemon in this example, were labeled as LPMP.

Lipid nanoparticle (LNP) formulation was prepared according to Example 1-2, composed of ionizable lipid:structural lipid:sterol:PEG-lipid at given molar ratios outlined in Table 2. Lipids were solubilized in ethanol. These lipids were mixed at the indicated molar ratios and diluted in ethanol (organic phase) to 5.5 mM total lipid concentration and the mRNA solution (aqueous phase) was prepared with RNAse-free water and 100 mM citrate buffer pH 3 for a final concentration of 50 mM citrate buffer. Formulations were maintained at ionizable lipid to mRNA N:P ratio of 3:1-15:1 (Table 2).

In some formulations, a lemon LPMP composition was formulated according to Example 1-2, composed of ionizable lipid:lemon lipid:sterol:PEG-lipid at given molar ratios outlined in Table 2. Lipids were solubilized in 4:1 DMF:ethanol. These lipids were mixed at the indicated molar ratios and diluted in ethanol (organic phase) to 5.5 mM total lipid concentration and the mRNA solution (aqueous phase) was prepared with RNAse-free water and 100 mM citrate buffer pH 3 for a final concentration of 50 mM citrate buffer. Formulations were maintained at ionizable lipid to mRNA N:P ratio of 3:1-15:1 (Table 2).

In some formulations, an E. coli LNMP composition was formulated according to Example 2, comprising ionizable lipid: E. coli polar lipids:sterol:PEG-lipid or ionizable lipid:(E. coli polar lipids+a neutral lipid):sterol:PEG-lipid at given molar ratios outlined in Table 2. The E. coli polar lipids were purchased from Avanti. As shown in Table 2, the neutral lipid used was DOPE or DSPC, and when used, was in a molar ratio of E. coli polar lipids:neutral lipid=10:10. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 3:1-15:1 (Table 2).

The lipid mix and mRNA solution were mixed at a 1:3 ratio by volume, respectively, on the NANOASSEMBLR® IGNITE™ (Precision Nanosystems) at a total flow rate of 14 mL/minute. The resulting formulations were then loaded into Slide-A-Lyzer G2 dialysis cassettes (10 k MWCO) and dialyzed against 1×PBS for 2 hours at room temperature. The PBS was refreshed, and the formulations were further dialyzed for at least 14 hours at 4° C. with gentle stirring. The dialyzed formulations were then collected and concentrated by centrifugation at 2000×g using AMICON® Ultra centrifugation filters (100 k MWCO). The concentrated formulations were characterized for size, polydispersity, and particle concentration using a Zetasizer Ultra (Malvern Panalytical) and for mRNA encapsulation efficiency using QUANT-IT™ RIBOGREEN® RNA Assay Kit (ThermoFisher Scientific).

TABLE 2
Characterizations of LNMP formulations

| | Ionizable:Structural: | | | | | | |
|---|---|---|---|---|---|---|---|
| | Sterol:PEG-lipid | | | | | N:P | |
| Formulation | Molar ratio | Ionizable | Structural | Sterol | PEG-lipid | Ratio | Cargo |
| C12-200 LNP | 35:16:46.5:2.5 | C12-200 | DOPE | Chol | DMG-PEG | 15:1 | SARS-COV-2 |
| | | | | | | | mRNA |
| 2275 LPMP | 35:20:42.5:2.5 | 2275 | Naturally derived | Chol | DMG-PEG | 6:1 | SARS-COV-2 |
| | | | lemon lipid | | | | mRNA |
| 2272 LPMP | 35:20:42.5:2.5 | 2272 | Naturally derived | Chol | DMG-PEG | 6:1 | SARS-COV-2 |
| | | | lemon lipid | | | | mRNA |
| 2248 LPMP | 35:20:42.5:2.5 | 2248 | Naturally derived | Chol | DMG-PEG | 12:1 | SARS-COV-2 |
| | | | lemon lipid | | | | mRNA |
| 2275 LNMP | 35:10 + 10:42.5:2.5 | 2275 | E. coli lipid (Avanti) + | Chol | DMG-PEG | 6:1 | SARS-COV-2 |
| | | | DOPE | | | | mRNA |
| 2272 LNMP | 35::10 + 10:42.5:2.5 | 2272 | E. coli lipid (Avanti) + | Chol | DMG-PEG | 6:1 | SARS-COV-2 |
| | | | DOPE | | | | mRNA |
| 2308 LNMP | 50:10 + 10:28.5:1.5 | 2308 | E. coli lipid (Avanti) + | Chol | DMG-PEG | 6:1 | SARS-COV-2 |
| | | | DSPC | | | | mRNA |
| 2335 LPMP | 50:20:28.5:1.5 | 2335 | Naturally derived | Chol | DMG-PEG | 3:1 | SARS-COV-2 |
| | | | lemon lipid | | | | mRNA |
| 2248 LNMP | 35:10 + 10:42.5:2.5 | 2248 | E. coli lipid (Avanti) + | Chol | DMG-PEG | 12:1 | SARS-COV-2 |
| | | | DOPE | | | | mRNA |

### Vaccination and Collection in Mice

8-9 week old C57BL/6 mice were utilized for vaccination screening efforts of the formulations provided in Table 2, with PBS and C12-200 LNP acting as controls. Mice were obtained from Jackson Laboratories and allowed to acclimate for one week prior to manipulations. Five C57BL/6 mice were evaluated for each formulation and buffer. The test samples were injected into mice on DO using the intramuscular (IM) route, in one upper thigh in the hind limb. On D14, terminal bleeds were collected via retro-orbital puncture into a heparin blood collection tube. Samples were centrifuged for 2000 g for 10 minutes, and plasma was transferred and stored at −80° C. Spleens were collected as well and placed in a RPMI solution. Splenocytes were isolated on a cell strainer and filtered on 70 μm filters before and after red blood cell lysis. Antigen specific T cells were evaluated via ELISPOT as described below. Samples were transiently stored at −80° C. before being transferred to liquid nitrogen.

Blood plasma collection. Blood samples were collected on D14, as described above.

Binding antibody titers evaluation by ELISA. The analysis of the antibody responses (IgG) against SARS-CoV-2 was performed to quantify the responses for mice antibodies targeting antigens of SARS-CoV-2 using an ELISA. IgG antibody titers against SARS-CoV-2 were determined by diluting plasma in 1/10 fold dilutions. Briefly, ELISA plates were coated with recombinant Spike RBD overnight. The following day, plates were blocked, samples were diluted, and all dilutions were added overnight. On day 3, detection antibodies and ELISA development were completed. Antibody titers were selected by choosing the last dilution where samples were no longer diluting.

ELISPOT cytokine evaluation of anti-SARS-CoV-2/S T cell responses. T cell responses against SARS-CoV-2 antigens were evaluated on D14 in mice using an IFNγ immunospot color ELISPOT assay. Splenocytes were isolated on a cell strainer and filtered on before and after red blood cell lysis. Live splenocytes were counted and their viability was assessed by acridine orange exclusion assay on a Luna cell counter. ELISPOT assays were run by stimulating the cells with SARS-CoV-2/S peptides pools overnight. The following day, the IFNγ producing cells were evaluated using the IFNγ color immunospot assay. The plates were analyzed using an Immunospot CTL-S6 Fluor analyzer.

TABLE 3
Treatment details

| | Number | Dose | | |
|---|---|---|---|---|
| Particle ID | of animals | Route | Dose Conc/Vol | Payload |
| C12-200 LNP | 5 | IM | 10ug/40uL | S mRNA |
| 2275 LPMP | 5 | | | |
| 2272 LPMP | 5 | | | |
| 2248 LPMP | 5 | | | |
| 2275 LNMP | 5 | | | |
| 2272 LNMP | 5 | | | |
| 2308 LNMP | 5 | | | |
| 2335 LPMP | 5 | | | |
| 2248 LNMP | 5 | | | |

In sum, five mice per experimental group were intramuscularly administered with a single dosage of LNMP/SARS-CoV-2 (containing S mRNA 10 μg), or LPMP/SARS-CoV-2 (containing S mRNA 10 μg) prepared according to Examples 1-3 and Table 2. Antibody response (IgG) was evaluated by indirect ELISA at D14.

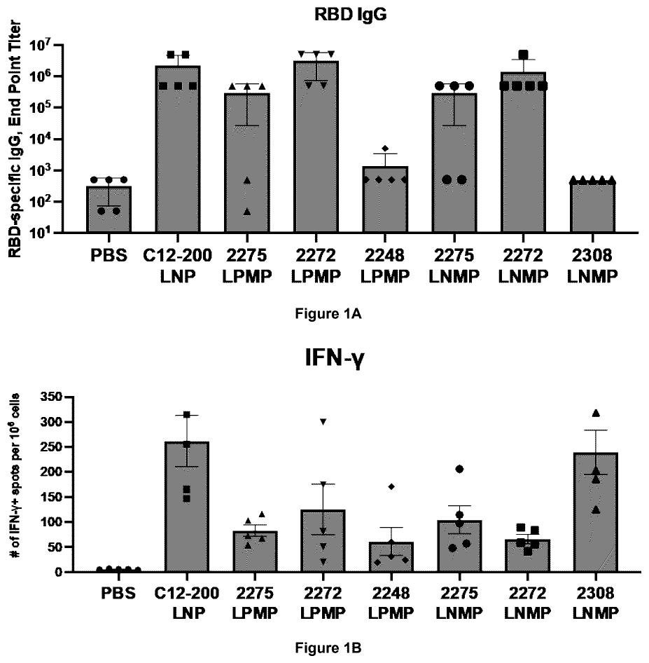

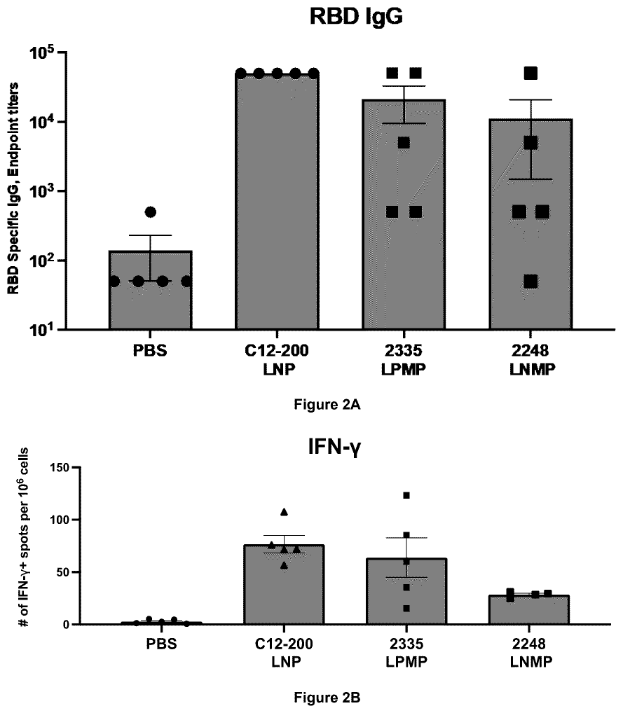

FIGS. 1A and 2A shows the levels of antibody (IgG) specific to the receptor binding domain (RBD) of SARS-CoV-2 in the blood of mice after 14 days, after a single dose intramuscular delivery of the LNMP or LPMP formulations (S mRNA 10 μg, Table 2) employing the novel ionizable lipids disclosed herein. N=5/group. Controls were PBS and C12-200 LNP (i.e., the LNP composition employing C12-200 as the ionizable lipid). N=5/group. The results indicate that a single dosage intramuscular vaccination of the majority of tested LNMP or LPMP formulations employing the novel ionizable lipids disclosed herein described in Table 2 were able to induce a high level of RBD-antigen specific IgG at 14 days after vaccination in the mice.

Anti-SARS-CoV-2/S T cell responses were evaluated in corresponding ELISPOT assay (IFNγ) at D14. FIGS. 1B and 2B show the number of SARS-CoV-2 S-specific T cells producing cytokine IFNγ per 10⁶ splenocytes in mice at 14 days, after a single dose intramuscular delivery of the LNMP or LPMP formulations (S mRNA 10 μg, Table 2) employing the novel ionizable lipids disclosed herein. N=5/group. Controls were PBS and C12-200 LNP (i.e., the LNP composition employing C12-200 as the ionizable lipid). N=5/group. The results indicate that a single dosage intramuscular vaccination of the majority of tested LNMP or LPMP formulations employing the novel ionizable lipids disclosed herein described in Table 2 induced high levels of IFNγ production in splenocytes, with the results of several LNMP or LPMP formulations comparable to that of C12-200 LNP.

### Example 4: Testing of LNMP/SARS-CoV-2 Formulations in Hamsters

If not specified, the LNP/mRNA or LNMP/mRNA formulations were prepared according to those described in Example 1-3. The experiment was designed to test the immunogenicity in hamsters of the candidate coronavirus vaccines comprising an mRNA of Table 1 encoding a coronavirus antigen (e.g., the spike (S) protein), such as a SARS-CoV-2 antigen. An example of the coding portion of the delivered cargo S protein mRNA is further described in Example 3. In this example, controls include commercial monovalent vaccines or bivalent vaccines that contain the S mRNA formulated within a conventional lipid nanoparticle composition (without natural lipids) containing either ALC-0315 ([(4-hydroxybutyl)azanediyl]di(hexane-6,1-diyl) bis(2-hexyldecanoate)) or SM-102 (heptadecan-9-yl 8-[2-hydroxyethyl-(6-oxo-6-undecoxyhexyl)amino]octanoate); a phospholipid; cholesterol; and a PEG-lipid. A commercial monovalent vaccine refers to the vaccine that contains the S mRNA targeting SARS-CoV-2 original strain, and a commercial bivalent vaccine refers to the vaccine that contains the S mRNA targeting SARS-CoV-2 original strain and Omicron BA.4/BA.5 strain.

### Formulation and Characterization of LNMP/mRNA

This example further describes the formulation of several LNMPs formulated with ionizable lipids, structural lipids (natural or natural combined with synthetic), sterols, and PEG lipids, to encapsulate mRNA (e.g., S mRNA) for LNMP/mRNA. LNMP compositions contained natural lipids extracted from a variety of natural sources, e.g., a plant source (such as lemon or soy), or a bacteria source (such as E. Coli), and were labeled accordingly.

In some formulations, a lemon LPMP composition was formulated according to Example 1-3, composed of ionizable lipid:lemon lipid:sterol:PEG-lipid at given molar ratios outlined in Table 4. These lipids were mixed at the indicated molar ratios and diluted in ethanol (organic phase) to 5.5 mM total lipid concentration and the mRNA solution (aqueous phase) was prepared with RNAse-free water and 100 mM citrate buffer pH 3 for a final concentration of 50 mM citrate buffer. Formulations were maintained at ionizable lipid to mRNA N:P ratio of 3:1-15:1 (Table 4).

In some formulations, an E. coli LNMP composition was formulated according to Example 2, comprising ionizable lipid: E. coli polar lipids:sterol:PEG-lipid at given molar ratios outlined in Table 4. In some cases, the E. coli polar lipids were purchased from Avanti. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 3:1-15:1 (Table 4).

In some formulations, a soy LPMP composition was formulated according to Example 2, comprising ionizable lipid:soy lipids:sterol:PEG-lipid at given molar ratios outlined in Table 4. In some cases, the soy lipids were purchased from Avanti. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 3:1-15:1 (Table 4).

The lipid mix and mRNA solution were mixed at a 1:3 ratio by volume, respectively, on the NANOASSEMBLR® IGNITE™ (Precision Nanosystems) at a total flow rate of 14 mL/minute. The resulting formulations were then loaded into Slide-A-Lyzer G2 dialysis cassettes (10 k MWCO) and dialyzed against 1×PBS for 2 hours at room temperature. The PBS was refreshed, and the formulations were further dialyzed for at least 14 hours at 4° C. with gentle stirring. The dialyzed formulations were then collected and concentrated by centrifugation at 2000×g using AMICON® Ultra centrifugation filters (100 k MWCO). The concentrated formulations were characterized for size, polydispersity, and particle concentration using a Zetasizer Ultra (Malvern Panalytical) and for mRNA encapsulation efficiency using QUANT-IT™ RIBOGREEN® RNA Assay Kit (ThermoFisher Scientific).

TABLE 4
Characterizations of LNMP formulations

| | Ionizable:Structural: | | | | | | |
|---|---|---|---|---|---|---|---|
| Formulation | Sterol:PEG-lipid | | | | | N:P | |
| ID | Molar ratio | Ionizable | Structural | Sterol | PEG-lipid | Ratio | Cargo |
| C12 LPMP | 35:50:12.5:2.5 | C12-200 | Naturally derived | Chol | DMG- | 15:1 | SARS-COV-2 |
| | | | lemon lipids | | PEG2k | | mRNA |
| 2425 LPMP 1 | 45:20:33.5:1.5 | 2425 | Naturally derived | Chol | DMG- | 12:1 | SARS-COV-2 |
| | | | lemon lipids | | PEG2k | | mRNA |
| 2356 LNMP | 35:20:42.5:2.5 | 2356 | E. coli polar lipids | Chol | DMG- | 6:1 | SARS-COV-2 |
| | | | | | PEG2k | | mRNA |
| 2425 LNMP | 45:20:33.5:1.5 | 2425 | E. coli polar lipids | Chol | DMG- | 12:1 | SARS-COV-2 |
| | | | | | PEG2k | | mRNA |
| 2425 LPMP 2 | 45:10:43.5:1.5 | 2425 | Soy PC | Chol | DMG- | 12:1 | SARS-COV-2 |
| | | | | | PEG2k | | mRNA |

### Vaccination and Collection in Hamsters Experiment 1

In one experiment, 6 experimental groups (n=12 hamsters/group) were intramuscularly dosed with the LNMPs, buffer, or vaccine control as shown in Table 5 on Day 0 and Day 23 (20 ug/dose). Blood was drawn on Day 23 and Day 42 prior to the respective boost and challenge. On Day 43, an intranasal SARS-CoV-2 challenge (6×10³ PFU original wuhan variant) was introduced to all hamsters. Half of each group was euthanized on D47 (4 days post infection) and the remaining hamsters of each group were euthanized on D50 (7 dpi). At terminal endpoints (4 dpi or 7 dpi), fluid and tissue samples were taken.

Blood collection. Whole blood was collected in serum separator tubes via retro-orbital bleed or a cardiac puncture. Blood samples were processed for serum. Briefly, blood was allowed to clot for 30 minutes to 1 hour at room temperature. Samples were then centrifuged for 5-10 minutes at 1000-1300×g. Serum was collected and stored at −20° C.

Nasal Lavage Collection. Nasal lavages were collected to measure IgA levels via ELISA. Animals will be sedated by inhalation of isoflurane as per BIOQUAL standard procedures. 0.3 mL of PBS was introduced into the upper nostril of the animals via catheter. A cryovial was placed under the animals' nose to collect drops of lavage solution.

Tissue Collection. Following the terminal bleeds, animals were necropsied for lungs and nasal turbinates, which were weighed whole and snap frozen in small sections for TCID50 and qRT-PCR. Alternatively, left lung lobes were collected from animals in 10% NBF for histopathological evaluation.

Spleen Sample Processing. Following the terminal bleeds, animals were necropsied for spleens. Spleens were stored in 5 mL of complete RPMI 1640 media and processed. Briefly, spleen samples were strained through a primed 70 μm cell strainer. Following cell isolation, samples were centrifuged at 500×g for 10 minutes and the supernatant was carefully removed. Single-cell suspensions of splenocytes were subjected to ACK lysis of erythrocytes and washed. The cell suspension was then filtered through a 100 μm MACs SmartStrainer (Miltenyi Biotec Cat. #: 130-110-917) into a new, sterile tube and centrifuged at 4 degrees Celsius at 500×g for 8 minutes. After the supernatant from the centrifuged cell suspension was removed, the cell pellet was resuspended in 1-2 mL of L-Glutamine CTL Media. The cells were kept briefly on ice before cell counting and resuspension in L-Glutamine CTL Media at 2.5×106 cells/mL.

IgA and IgG ELISAs. A standard indirect ELISA was performed to analyze serum samples from D23, 42, and 49 for binding antibodies to the SARS-CoV-2 Spike (S) protein. Briefly, ELISA plates were coated with recombinant Spike RBD overnight. The following day, plates were blocked, samples were diluted, and all dilutions were added and incubated. After incubation, detection antibodies and ELISA development were completed. IgA antibody titers were defined as reciprocal of the dilutions that generate a specific cut-off value for OD450 on the linear part of the titration curve. IgG antibody titers were selected by choosing the last dilution where samples were no longer diluting.

Plaque Reduction Neutralization Test (PRNT). The SARS-CoV-2 PRNT assays were conducted on serum samples from D23, 42, 46, and 49. Vero E6 cells were plated in growth media (DMEM+10% FBS+gentamicin). The cells were incubated at 37° C., 5.0% CO₂ until the cells reached 80-100% confluency the following day. Heat-inactivated serum samples or positive control virus in diluent media (DMEM+2% FBS+gentamicin) was added to the cells and mixed via pipetting at varying dilutions, then virus dilution was added to the sample and positive control wells. Plates were sealed and incubated for 1 h. Media was removed, titrated samples/virus dilutions were added in duplicate, and the plates were then incubated for 1 h again. Overlay methylcellulose media was then added and plates were incubated for 3 days. After infection, the methylcellulose overlay medium was removed, and the plates were washed and fixed. After fixation, the cell monolayers were stained with 0.2% crystal violet (30% MeOH, 80% dH2O) for 30 minutes at room temperature. The plates were washed and dried. Plaques in each well were recorded and the IC50 titers were calculated based on the average number of plaques detected in the virus control wells.

TCID50 Assay. TCID50 assay was performed on processed lung and nasal turbinates samples to determine infectious viral titers. Plates were seeded with TMPRSS2 cells and incubated until 80-100% confluent. Growth media was aspirated and replaced with 180 μL of diluent media (DMEM+2% FBS+gentamicin) per well. 20 μL of processed tissue sample (or positive/negative control) was added, mixed via pipetting, and then serially diluted down the rows in a 10 fold dilution. Plates were incubated for 4 days. After incubation, the presence of cytopathic effects (CPE) were denoted in the TCID Assay forms as a plus (+) and the absence of CPE as a minus (−); wherein non-infected cells were defined as having a clear confluent cell layer. The TCID50 value was calculated using the Read-Muench formula.

qRT-PCR. Quantitative RT-PCR analysis for viral load was performed on lung and nasal turbinate samples. The qRT-PCR assay for SARS-CoV-2 genomic RNA (gRNA) utilized primers and a probe specifically designed to amplify and bind to a conserved region of the Nucleocapsid gene of coronavirus; the signal was compared to a known standard curve and calculated to give copies per gram of tissue. The qRT-PCR assay for SARS-CoV-2 subgenomic RNA (sgRNA) utilized primers and a probe specifically designed to amplify and bind to a region of the E gene of messenger RNA from SARS-COV-2, which is not packaged into the virion. The signal was compared to a known standard curve of plasmid containing the sequence of part of the messenger RNA including the part that is not in the virus and calculated to give copies per gram of tissue.

ELISPOT cytokine evaluation of anti-SARS-CoV-2/S T cell responses. T cell responses against SARS-CoV-2 antigens were evaluated in hamsters using an IFNγ immunospot color ELISPOT assay. Splenocytes were isolated on a cell strainer and filtered on before and after red blood cell lysis. Live splenocytes were counted and their viability was assessed by acridine orange exclusion assay on a Luna cell counter. ELISPOT assays were run by stimulating the cells with SARS-CoV-2/S peptides pools overnight. The following day, the IFNγ producing cells were evaluated using the IFNγ color immunospot assay. The plates were analyzed using an Immunospot CTL-S6 Fluor analyzer.

TABLE 5
Treatment details of Hamster Experiment 1

| | Number | Dose | | |
|---|---|---|---|---|
| Particle ID | of animals | Route | Dose Conc/Vol | Payload |
| C12 LPMP | 12 | IM | 20ug/200uL | S |
| 2425 LPMP 1 | 12 | | | mRNA |
| 2356 LNMP | 12 | | | |
| 2425 LNMP | 12 | | | |
| Commercial | 12 | | | |
| Vaccine | | | | |
| Control | | | | |
| Buffer | 12 | | | |

In sum, 12 hamsters per experimental group were intramuscularly administered with a dosage of various LNMPs/SARS-CoV-2 (containing S mRNA 20 μg) prepared according to Examples 1-4 and Table 4, buffer, or a commercially available vaccine control at DO and D23. Antibody responses (IgA and IgG) in the hamsters administered various LNMP formulations (S mRNA 20 μg, see the LNMP formulations in Table 4) were evaluated by ELISA at D23 after one dose and D42 after two doses (prime and booster doses). For the commercial vaccine control in hamsters Experiment 1, a commercially available bivalent vaccine was used in the first dose and in the second dose based on the dose level listed in Table 5.

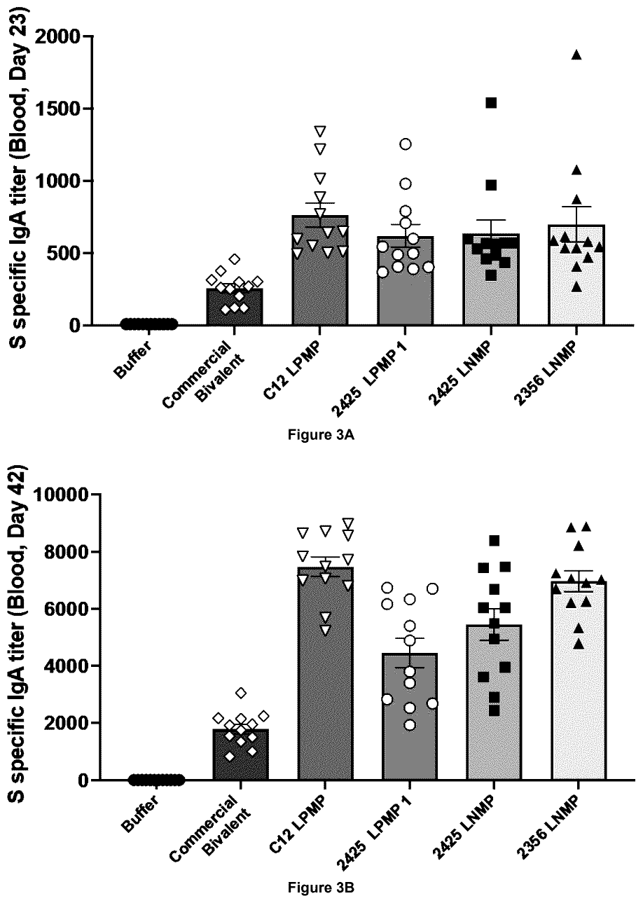

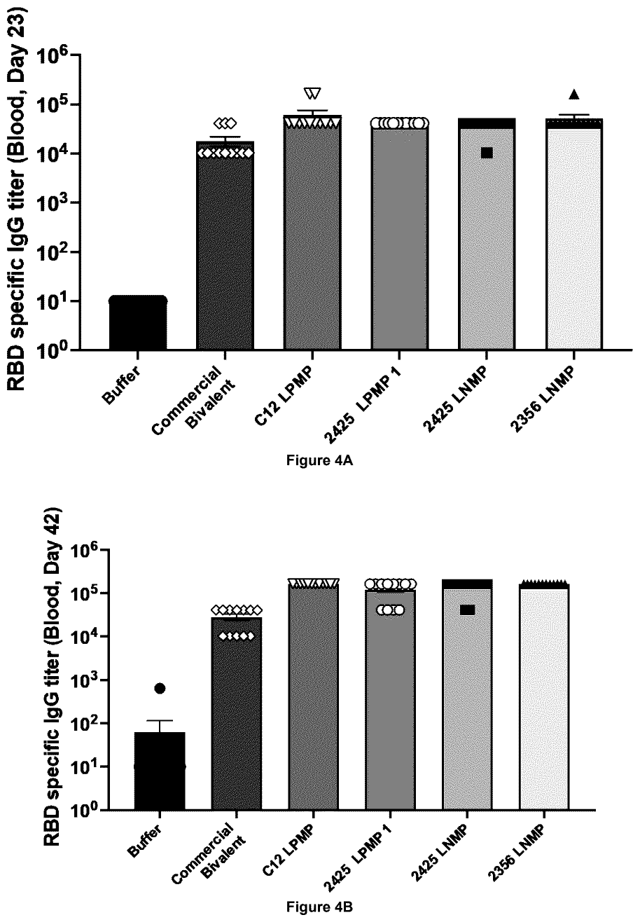

FIGS. 3A and 3B show the levels of S-specific IgA in the blood of hamsters after one or two intramuscular doses of the LNMP formulations (S mRNA 20 μg, Table 4) on D23 (after one dose) and D42 (after two doses). The results indicate that a single dosage intramuscular vaccination of all experimental LNMP formulations described in Table 4 (including the formulation employing C12-200 as the ionizable lipid, as well as the formulations employing various novel ionizable lipids listed in Table 4) were able to induce a high level of S-antigen specific IgA at 23 days after vaccination in the hamsters, and were much higher than the commercially available bivalent vaccine. The LNMP formulations employing the novel ionizable lipids produced results comparable to the formulation employing C12-200 as the ionizable lipid. These results held true after two doses at D42 as well.

FIGS. 4A and 4B show the levels of RBD-specific IgG in the blood of hamsters after one or two intramuscular doses of the LNMP formulations (S mRNA 20 μg, Table 4) on D23 (after one dose) and D42 (after two doses). The results indicate that a single dosage intramuscular vaccination of all experimental LNMP formulations described in Table 4 (including the formulation employing C12-200 as the ionizable lipid, as well as the formulations employing various novel ionizable lipids listed in Table 4) were able to induce a high level of RBD-antigen specific IgG at 23 days after vaccination in the hamsters and were higher than the commercially available bivalent vaccine. The LNMP formulations employing the novel ionizable lipids produced results comparable to the formulation employing C12-200 as the ionizable lipid. These results held true after two doses at D42 as well.

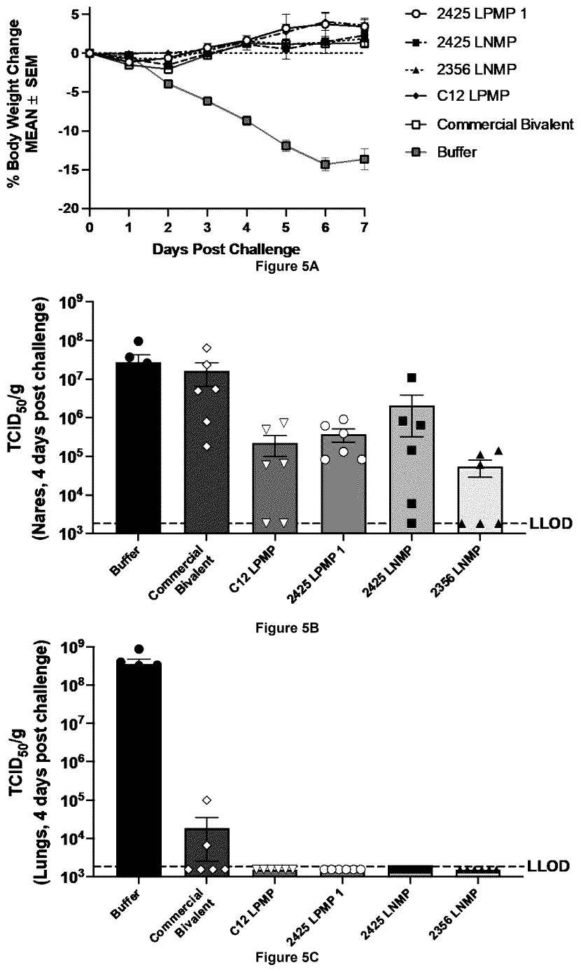

FIG. 5A shows the percentage body weight change as mean across all groups of hamsters after two intramuscular doses of various LNMP formulations (S mRNA 20 μg, Table 4) over days after a SARS-CoV-2 challenge. The results show that the LNMPs formulations (including the formulation employing C12-200 as the ionizable lipid, as well as the formulations employing various novel ionizable lipids listed in Table 4) provided protection against a SARS-CoV-2 challenge, and were comparable to or better than the commercial vaccine, as measured by body weight. FIG. 5B shows the infectious viral titers in nares across all groups of hamsters four days post-challenge. The results indicate that the LNMPs formulations (including the formulation employing C12-200 as the ionizable lipid, as well as the formulations employing various novel ionizable lipids listed in Table 4) provided protection against a SARS-CoV-2 challenge, and were comparable to or better than the commercial vaccine. FIG. 5C shows the infectious viral titers in lungs across all groups of hamsters four days post-challenge. Measured values that produced less TCID50 than the lower limit of detection (LLOD) were assigned the LLOD value. The results indicate that the LNMPs formulations (including the formulation employing C12-200 as the ionizable lipid, as well as the formulations employing various novel ionizable lipids listed in Table 4) provided protection against a SARS-CoV-2 challenge that was comparable to or better than the commercial vaccine, with undetectable lung viral load in several experimental groups.

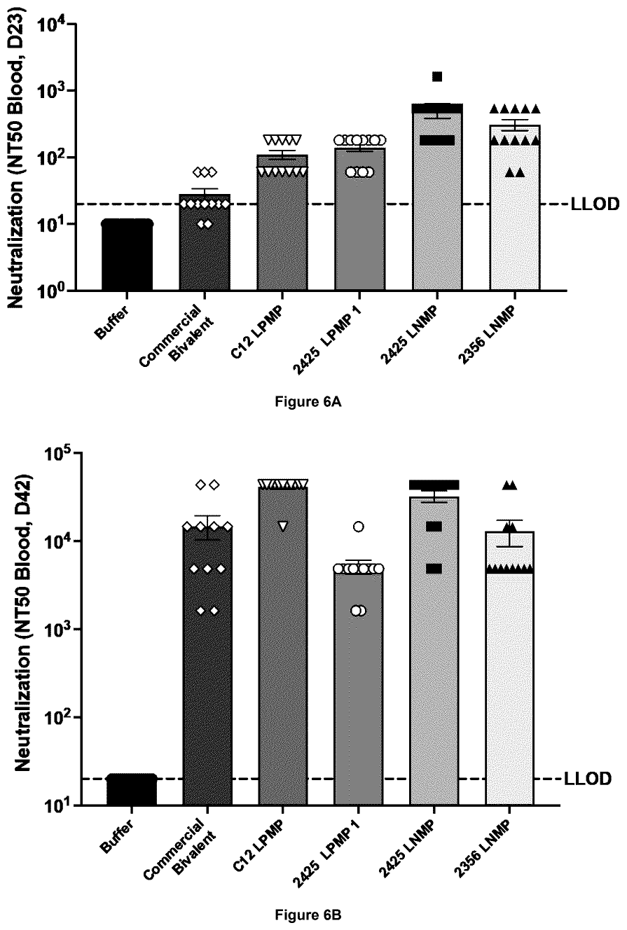

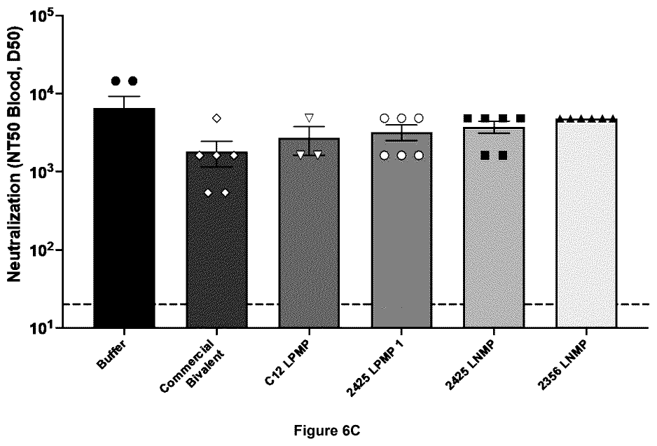

FIG. 6A-C show the levels of neutralizing antibody titer against the SARS-CoV-2 original strain in the blood of the hamsters after a single intramuscular vaccination of the LNMP formulations (S mRNA 20 μg, Table 4) at 23 days (FIG. 6A), after a prime and boost vaccination at 42 days (FIG. 6B), and post-boost and 7 days post-challenge at 50 days (FIG. 6C). The results show that all the LNMP formulations (including the formulation employing C12-200 as the ionizable lipid, as well as the formulations employing various novel ionizable lipids listed in Table 4) had a neutralization comparable to or higher than the commercially available vaccine after one or two intramuscular dose. The LNMP formulations employing the novel ionizable lipids had a higher neutralization than the formulation employing C12-200 as the ionizable lipid after a single intramuscular vaccination at D23 and post-boost and 7 days post-challenge at D50.

### Vaccination and Dosing in Hamsters Experiment 2

In another experiment, 3 experimental groups (n=14 hamsters/group) were intramuscularly dosed with a commercial monovalent vaccine (1 ug) or either 2425 LPMP 2/SARS-CoV-2 (soy LPMP, containing S mRNA, 10 ug) or 2425 LPMP 2/SARS-CoV-2 (soy LPMP, containing S mRNA, 1 ug) on Day 0 and Day 21. The 2425 LPMP 2 formulation is shown in Table 4. A control group of 18 hamsters was not dosed (pooled naïve hamsters). Blood was drawn on Day 21, prior to their vaccination, and Day 42, prior to when an intranasal SARS-CoV-2 challenge was introduced to all hamsters. Processing of samples and assay procedures were followed as described above in Example 4.

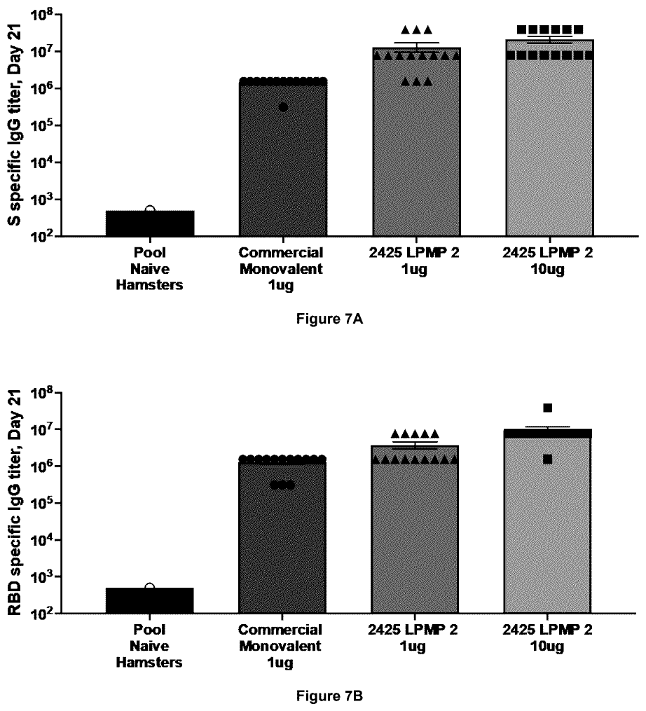

FIG. 7A shows the level of S-specific IgG in the blood of hamsters after one intramuscular dose of 2425 LPMP 2/SARS-CoV-2 (soy LPMP, containing S mRNA, 10 ug) or 2425 LPMP 2/SARS-CoV-2 (soy LPMP, containing S mRNA, 1 ug) on Day 21 post-dose. FIG. 7B shows the level of RBD-specific IgG in the blood of hamsters after one intramuscular dose of 2425 LPMP 2/SARS-CoV-2 (soy LPMP, containing S mRNA, 10 ug) or 2425 LPMP 2/SARS-CoV-2 (soy LPMP, containing S mRNA, 1 ug) on Day 21 post-dose. The results indicate that both doses of the LPMP formulations employing the novel ionizable lipid (Lipid No. 2425) induced high IgG binding titers and were higher than titers induced by the commercial monovalent vaccine.

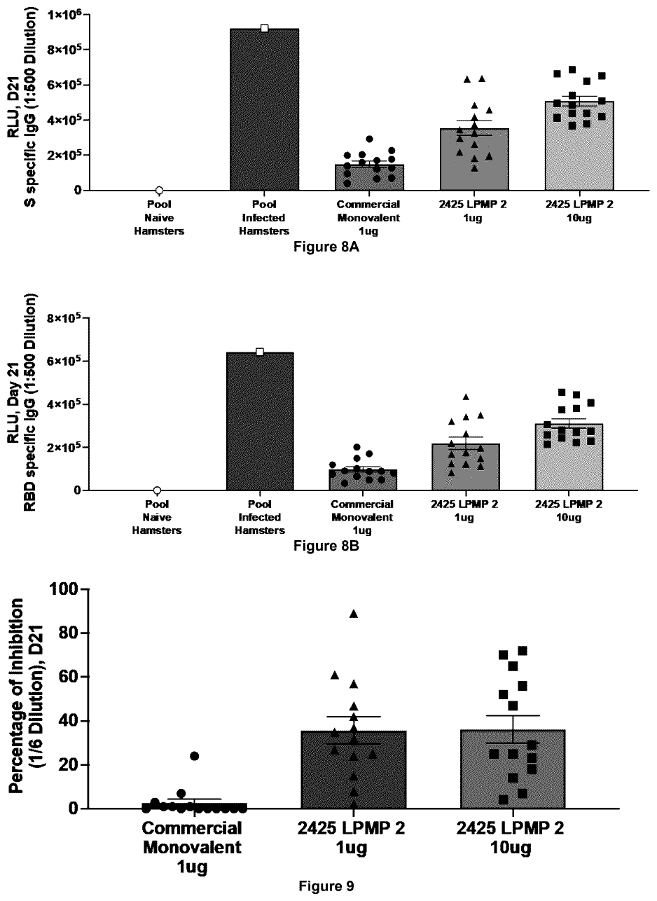

FIG. 8A shows the amount of S-specific IgG in the blood of hamsters after one intramuscular dose of 2425 LPMP 2/SARS-CoV-2 (soy LPMP, containing S mRNA, 10 ug) or 2425 LPMP 2/SARS-CoV-2 (soy LPMP, containing S mRNA, 1 ug) on Day 21 post-dose. FIG. 8B shows the amount of RBD-specific IgG in the blood of hamsters after one intramuscular dose of 2425 LPMP 2/SARS-CoV-2 (soy LPMP, containing S mRNA, 10 ug) or 2425 LPMP 2/SARS-CoV-2 (soy LPMP, containing S mRNA, 1 ug) on Day 21 post-dose. The results show that both doses of the LPMP formulations employing the novel ionizable lipid (Lipid No. 2425) induced higher amounts of SARS-CoV-2 specific IgG antibodies than the commercial monovalent vaccine.

FIG. 9 shows the percentage of inhibition in hamsters after one intramuscular dose of 2425 LPMP 2/SARS-CoV-2 (soy LPMP, containing S mRNA, 10 ug) or 2425 LPMP 2/SARS-CoV-2 (soy LPMP, containing S mRNA, 1 ug) on Day 21 post-dose. The results indicate that a single dose of LPMP formulations employing the novel ionizable lipid (Lipid No. 2425) at either dosage (1 ug or 10 ug) induced a higher increase in the percentage of inhibition, compared to the commercial monovalent vaccine (1 ug).

### Example 5: Immunogenicity of LNMP/SARS-CoV-2 Formulations in Mice

If not specified, the LNP/mRNA or LNMP/mRNA formulations were prepared according to those described in Example 1-3. The experiment was designed to test the immunogenicity in mice of the candidate coronavirus vaccines comprising an mRNA of Table 1 encoding a coronavirus antigen (e.g., the spike (S) protein), such as a SARS-CoV-2 antigen. An example of the coding portion of the delivered cargo S protein mRNA is further described in Example 3.

### Formulation and Characterization of LNMP/mRNA

This example further describes the formulation of several LNMPs formulated with ionizable lipids, structural lipids (natural or natural combined with synthetic), sterols, and PEG lipids, to encapsulate mRNA (e.g., S mRNA) for LNMP/mRNA. LNMP compositions contained natural lipids extracted from a variety of natural sources, e.g., a plant source (such as lemon or soy), or a bacteria source (such as E. Coli), and were labeled accordingly.

Lipid nanoparticle (LNP) formulation was prepared according to Example 1-3, composed of ionizable lipid:structural lipid:sterol:PEG-lipid at given molar ratios outlined in Table 6. Lipids were solubilized in ethanol. These lipids were mixed at the indicated molar ratios and diluted in ethanol (organic phase) to 5.5 mM total lipid concentration and the mRNA solution (aqueous phase) was prepared with RNAse-free water and 100 mM citrate buffer pH 3 for a final concentration of 50 mM citrate buffer. Formulations were maintained at ionizable lipid to mRNA N:P ratio of 3:1-15:1 (Table 6).

In some formulations, a lemon LPMP composition was formulated according to Example 1-3, composed of ionizable lipid:lemon lipid:sterol:PEG-lipid at given molar ratios outlined in Table 6. These lipids were mixed at the indicated molar ratios and diluted in ethanol (organic phase) to 5.5 mM total lipid concentration and the mRNA solution (aqueous phase) was prepared with RNAse-free water and 100 mM citrate buffer pH 3 for a final concentration of 50 mM citrate buffer. Formulations were maintained at ionizable lipid to mRNA N:P ratio of 3:1-15:1 (Table 6).

In some formulations, an E. coli LNMP composition was formulated according to Example 2, comprising ionizable lipid: E. coli polar lipids:sterol:PEG-lipid at given molar ratios outlined in Table 6. In some cases, the E. coli polar lipids were purchased from Avanti. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 3:1-15:1 (Table 6).

In some formulations, a soy LPMP composition was formulated according to Example 2, comprising ionizable lipid:soy lipids:sterol:PEG-lipid at given molar ratios outlined in Table 6. In some cases, the soy lipids were purchased from Avanti. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 3:1-15:1 (Table 6).

The lipid mix and mRNA solution were mixed at a 1:3 ratio by volume, respectively, on the NANOASSEMBLR® IGNITE™ (Precision Nanosystems) at a total flow rate of 14 mL/minute. The resulting formulations were then loaded into Slide-A-Lyzer G2 dialysis cassettes (10 k MWCO) and dialyzed against 1×PBS for 2 hours at room temperature. The PBS was refreshed, and the formulations were further dialyzed for at least 14 hours at 4° C. with gentle stirring. The dialyzed formulations were then collected and concentrated by centrifugation at 2000×g using AMICON® Ultra centrifugation filters (100 k MWCO). The concentrated formulations were characterized for size, polydispersity, and particle concentration using a Zetasizer Ultra (Malvern Panalytical) and for mRNA encapsulation efficiency using QUANT-IT™ RIBOGREEN® RNA Assay Kit (ThermoFisher Scientific).

TABLE 6
Characterizations of LNMP formulations

| | Ionizable:Structural: | | | | | | |
|---|---|---|---|---|---|---|---|
| Formulation | Sterol:PEG-lipid | | | | | N:P | |
| ID | Molar ratio | Ionizable | Structural | Sterol | PEG-lipid | Ratio | Cargo |
| LNP 1 | 50:10:38.5:1.5 | Lipid 8 | DSPC | Chol | DMG- | 6:1 | SARS-COV-2 |
| | | | | | PEG2k | | mRNA |
| 2356 LPMP | 35:20:42.5:2.5 | 2356 | Soy PC | Chol | DMG- | 6:1 | SARS-COV-2 |
| | | | | | PEG2k | | mRNA |
| 2356 LNMP | 35:20:42.5:2.5 | 2356 | E. coli polar | Chol | DMG- | 6:1 | SARS-COV-2 |
| | | | lipids | | PEG2k | | mRNA |
| 2425 LNMP | 45:20:33.5:1.5 | 2425 | E. coli polar | Chol | DMG- | 12:1 | SARS-COV-2 |
| | | | lipids | | PEG2k | | mRNA |
| 2425 LPMP 2 | 45:10:43.5:1.5 | 2425 | Soy PC | Chol | DMG- | 12:1 | SARS-COV-2 |
| | | | | | PEG2k | | mRNA |

### Vaccination and Collection in Mice

In this experiment, experimental groups (n=6 mice/group) were intramuscularly dosed with the LNMPs or LNP control on Day 0 (10 ug/40 uL, Table 6). On Day 7 and on Day 10, spleens and ipsilateral inguinal and ipsilateral popliteal lymph nodes were collected and processed for flow cytometry. Briefly, lymph nodes were pooled and kept on ice. LNs were digested using a collagenase solution at 37° C. for 10 minutes. Then the samples were washed and strained through a 70 um cell strainer. The samples were centrifuged, incubated with FC block and then stained. Germinal B cells were identified as CD19+/GL7+/CD95+/B220+. Class switched B cells were identified as CD19+/GL7+/CD95+/B220+/IgD−/IgG+. Follicular T cells were identified as CD4+/CXCR5+/PD-1+.

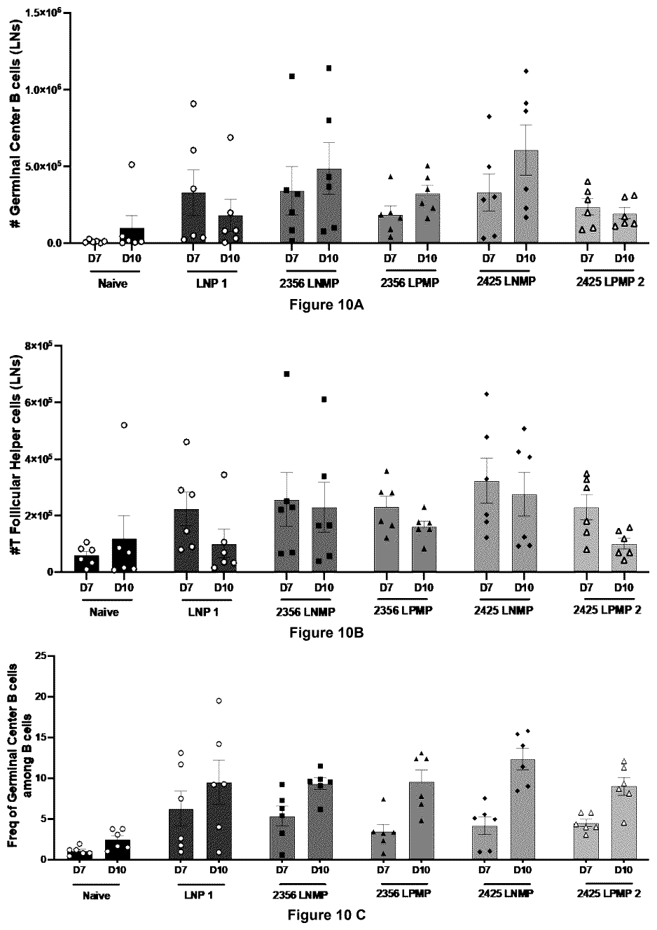

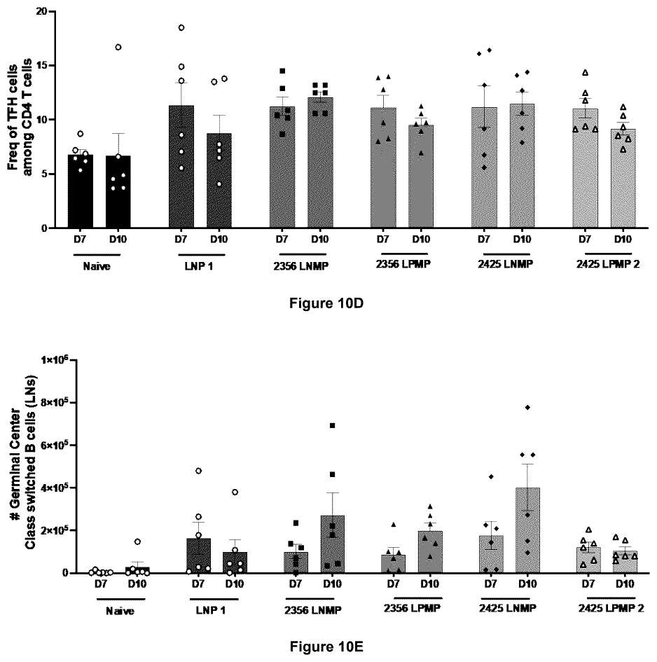

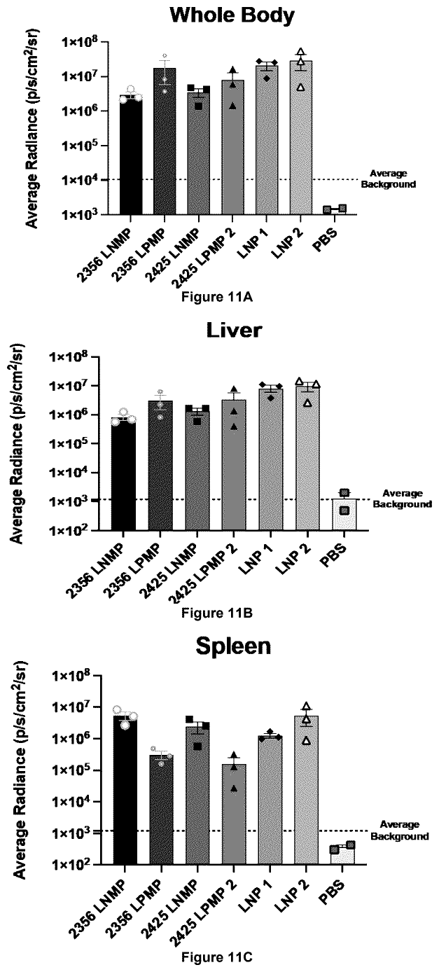

Each experimental group (2356 LPMP, 2356 LNMP, 2425 LNMP, and 2425 LPMP 2) was intramuscularly administered with a dosage of LNMPs/SARS-CoV-2 (containing S mRNA 10 μg/40 uL) prepared according to Examples 1-5 and Table 6. LNP 1 shown in Table 6 or naïve mice were used as controls. One cohort for each group was euthanized on Day 7 or Day 10, and spleens and lymph nodes were collected. The results are shown in FIGS. 10A-10E.

FIGS. 10A and 10B show the absolute number of germinal center B cells and of T follicular helper cells, respectively, in pooled lymph nodes per group after one intramuscular dose of various LNMP formulations (S mRNA 10 μg/40 μL) on D7 and D10, and FIGS. 10C and D show the frequency of germinal center B cells among B cells and the frequency of T follicular helper cells among CD4 T cells, respectively, in pooled lymph nodes per group after one intramuscular dose of various LNMP formulations (S mRNA 10 μg/40 μL) on D7 and D10. The results indicate that a single intramuscular dose of LNMP formulations employing various novel ionizable lipids as described herein induced germinal centers 7 and 10 days post-dose, comparable to or better than the LNP 1 control. In general, the results of LNMP formulations employing various novel ionizable lipids were better than the LNP 1 control on D10. FIG. 10E shows the absolute number of class switched B cells in pooled lymph nodes per group on D7 and D10 after one intramuscular dose of various LNMP formulations (S mRNA 10 μg/40 μL). The results indicate that a single intramuscular dose of LNMP formulations employing various novel ionizable lipids as described herein induced robust class switching of B cells, comparable to or better than the LNP 1 control. In general, the results of LNMP formulations employing various novel ionizable lipids were better than the LNP 1 control on D10.

### Example 6: Biodistribution of LNMP Formulations

If not specified, the LNP/mRNA or LNMP/mRNA formulations were prepared according to those described in Example 1-3. The experiment was designed to test the biodistribution in mice of the candidate vaccine formulations comprising an mRNA of 1:1 fLuc:EPO. An example of the coding portion of the EPO mRNA is as follows:

(SEQ ID NO: 29)
AUGGGCGUGCACGAGUGCCCCGCCUGGCUGUGGCUGCUGCUGAGCCUGCU
GAGCCUGCCCCUGGGCCUGCCCGUGCUGGGCGCCCCCCCCCGGCUGAUCU
GCGACAGCCGGGUGCUGGAGCGGUACCUGCUGGAGGCCAAGGAGGCCGAG
AACAUCACCACCGGCUGCGCCGAGCACUGCAGCCUGAACGAGAACAUCAC
CGUGCCCGACACCAAGGUGAACUUCUACGCCUGGAAGCGGAUGGAGGUGG
GCCAGCAGGCCGUGGAGGUGUGGCAGGGCCUGGCCCUGCUGAGCGAGGCC
GUGCUGCGGGGCCAGGCCCUGCUGGUGAACAGCAGCCAGCCCUGGGAGCC
CCUGCAGCUGCACGUGGACAAGGCCGUGAGCGGCCUGCGGAGCCUGACCA
CCCUGCUGCGGGCCCUGGGCGCCCAGAAGGAGGCCAUCAGCCCCCCCGAC
GCCGCCAGCGCCGCCCCCCUGCGGACCAUCACCGCCGACACCUUCCGGAA
GCUGUUCCGGGUGUACAGCAACUUCCUGCGGGGCAAGCUGAAGCUGUACA
CCGGCGAGGCCUGCCGGACCGGCGACCGGUGA

An example of the coding portion of the Fluc mRNA (TriLink, CleanCap Fluc mRNA) is as follows:

(SEQ ID NO: 30)
AUGGAGGACGCCAAGAACAUCAAGAAGGGCCCCGCCCCCUUCUACCCCCU
GGAGGACGGCACCGCCGGCGAGCAGCUGCACAAGGCCAUGAAGCGGUACG
CCCUGGUGCCCGGCACCAUCGCCUUCACCGACGCCCACAUCGAGGUGGAC
AUCACCUACGCCGAGUACUUCGAGAUGAGCGUGCGGCUGGCCGAGGCCAU
GAAGCGGUACGGCCUGAACACCAACCACCGGAUCGUGGUGUGCAGCGAGA
ACAGCCUGCAGUUCUUCAUGCCCGUGCUGGGCGCCCUGUUCAUCGGCGUG
GCCGUGGCCCCCGCCAACGACAUCUACAACGAGCGGGAGCUGCUGAACAG
CAUGGGCAUCAGCCAGCCCACCGUGGUGUUCGUGAGCAAGAAGGGCCUGC
AGAAGAUCCUGAACGUGCAGAAGAAGCUGCCCAUCAUCCAGAAGAUCAUC
AUCAUGGACAGCAAGACCGACUACCAGGGCUUCCAGAGCAUGUACACCUU
CGUGACCAGCCACCUGCCCCCCGGCUUCAACGAGUACGACUUCGUGCCCG
AGAGCUUCGACCGGGACAAGACCAUCGCCCUGAUCAUGAACAGCAGCGGC
AGCACCGGCCUGCCCAAGGGCGUGGCCCUGCCCCACCGGACCGCCUGCGU
GCGGUUCAGCCACGCCCGGGACCCCAUCUUCGGCAACCAGAUCAUCCCCG
ACACCGCCAUCCUGAGCGUGGUGCCCUUCCACCACGGCUUCGGCAUGUUC
ACCACCCUGGGCUACCUGAUCUGCGGCUUCCGGGUGGUGCUGAUGUACCG
GUUCGAGGAGGAGCUGUUCCUGCGGAGCCUGCAGGACUACAAGAUCCAGA
GCGCCCUGCUGGUGCCCACCCUGUUCAGCUUCUUCGCCAAGAGCACCCUG
AUCGACAAGUACGACCUGAGCAACCUGCACGAGAUCGCCAGCGGCGGCGC
CCCCCUGAGCAAGGAGGUGGGCGAGGCCGUGGCCAAGCGGUUCCACCUGC
CCGGCAUCCGGCAGGGCUACGGCCUGACCGAGACCACCAGCGCCAUCCUG
AUCACCCCCGAGGGCGACGACAAGCCCGGCGCCGUGGGCAAGGUGGUGCC
CUUCUUCGAGGCCAAGGUGGUGGACCUGGACACCGGCAAGACCCUGGGCG
UGAACCAGCGGGGCGAGCUGUGCGUGCGGGGCCCCAUGAUCAUGAGCGGC
UACGUGAACAACCCCGAGGCCACCAACGCCCUGAUCGACAAGGACGGCUG
GCUGCACAGCGGCGACAUCGCCUACUGGGACGAGGACGAGCACUUCUUCA
UCGUGGACCGGCUGAAGAGCCUGAUCAAGUACAAGGGCUACCAGGUGGCC
CCCGCCGAGCUGGAGAGCAUCCUGCUGCAGCACCCCAACAUCUUCGACGC
CGGCGUGGCCGGCCUGCCCGACGACGACGCCGGCGAGCUGCCCGCCGCCG
UGGUGGUGCUGGAGCACGGCAAGACCAUGACCGAGAAGGAGAUCGUGGAC
UACGUGGCCAGCCAGGUGACCACCGCCAAGAAGCUGCGGGGCGGCGUGGU
GUUCGUGGACGAGGUGCCCAAGGGCCUGACCGGCAAGCUGGACGCCCGGA
AGAUCCGGGAGAUCCUGAUCAAGGCCAAGAAGGGCGGCAAGAUCGCCGUG
UGA

### Formulation and Characterization of LNMP/mRNA

This example further describes the formulation of several LNMPs formulated with ionizable lipids, structural lipids (natural or natural combined with synthetic), sterols, and PEG lipids, to encapsulate mRNA (e.g., fLuc:EPO mRNA) for LNMP/mRNA. LNMP compositions contained natural lipids extracted from a variety of natural sources, e.g., a plant source (such as lemon or soy), or a bacteria source (such as E. Coli), and were labeled accordingly.

Lipid nanoparticle (LNP) formulation was prepared according to Example 1-3, composed of ionizable lipid:structural lipid:sterol:PEG-lipid at given molar ratios outlined in Table 7. Lipids were solubilized in ethanol. These lipids were mixed at the indicated molar ratios and diluted in ethanol (organic phase) to 5.5 mM total lipid concentration and the mRNA solution (aqueous phase) was prepared with RNAse-free water and 100 mM citrate buffer pH 3 for a final concentration of 50 mM citrate buffer. Formulations were maintained at ionizable lipid to mRNA N:P ratio of 3:1-15:1 (Table 7).

In some formulations, a lemon LPMP composition was formulated according to Example 1-3, composed of ionizable lipid:lemon lipid:sterol:PEG-lipid at given molar ratios outlined in Table 7. These lipids were mixed at the indicated molar ratios and diluted in ethanol (organic phase) to 5.5 mM total lipid concentration and the mRNA solution (aqueous phase) was prepared with RNAse-free water and 100 mM citrate buffer pH 3 for a final concentration of 50 mM citrate buffer. Formulations were maintained at ionizable lipid to mRNA N:P ratio of 3:1-15:1 (Table 7).

In some formulations, an E. coli LNMP composition was formulated according to Example 2, comprising ionizable lipid: E. coli polar lipids:sterol:PEG-lipid at given molar ratios outlined in Table 7. In some cases, the E. coli polar lipids were purchased from Avanti. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 3:1-15:1 (Table 7).

In some formulations, a soy LPMP composition was formulated according to Example 2, comprising ionizable lipid:soy lipids:sterol:PEG-lipid at given molar ratios outlined in Table 7. In some cases, the soy lipids were purchased from Avanti. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 3:1-15:1 (Table 7).

The lipid mix and mRNA solution were mixed at a 1:3 ratio by volume, respectively, on the NANOASSEMBLR® IGNITE™ (Precision Nanosystems) at a total flow rate of 14 mL/minute. The resulting formulations were then loaded into Slide-A-Lyzer G2 dialysis cassettes (10 k MWCO) and dialyzed against 1×PBS for 2 hours at room temperature. The PBS was refreshed, and the formulations were further dialyzed for at least 14 hours at 4° C. with gentle stirring. The dialyzed formulations were then collected and concentrated by centrifugation at 2000×g using AMICON® Ultra centrifugation filters (100 k MWCO). The concentrated formulations were characterized for size, polydispersity, and particle concentration using a Zetasizer Ultra (Malvern Panalytical) and for mRNA encapsulation efficiency using QUANT-IT™ RIBOGREEN® RNA Assay Kit (ThermoFisher Scientific).

TABLE 7
Characterizations of LNMP formulations

| | Ionizable:Structural: | | | | | | |
|---|---|---|---|---|---|---|---|
| Formulation | Sterol:PEG-lipid | | | | PEG- | N:P | |
| ID | Molar ratio | Ionizable | Structural | Sterol | lipid | Ratio | Cargo |
| LNP 1 | 50:10:38.5:1.5 | Lipid 8 | DSPC | Chol | DMG- | 6:1 | 1:1 fLuc: EPO |
| | | | | | PEG2k | | mRNA |
| LNP 2 | 47.3:9.6:41.4:1.7 | ALC-315 | DSPC | Chol | DMG- | 6:1 | 1:1 fLuc:EPO |
| | | | | | PEG2k | | mRNA |
| 2356 LPMP | 35:20:42.5:2.5 | 2356 | Soy PC | Chol | DMG- | 6:1 | 1:1 fLuc:EPO |
| | | | | | PEG2k | | mRNA |
| 2356 LNMP | 35:20:42.5:2.5 | 2356 | E. coli polar | Chol | DMG- | 6:1 | 1:1 fLuc:EPO |
| | | | lipids | | PEG2k | | mRNA |
| 2425 LNMP | 45:20:33.5:1.5 | 2425 | E. coli polar | Chol | DMG- | 12:1 | 1:1 fLuc:EPO |
| | | | lipids | | PEG2k | | mRNA |
| 2425 LPMP 2 | 45:10:43.5:1.5 | 2425 | Soy PC | Chol | DMG- | 12:1 | 1:1 fLuc:EPO |
| | | | | | PEG2k | | mRNA |

### Bioluminescence Screening

8-9 week old Balb/c mice were utilized for bioluminescence-based screening efforts of the formulations provided in Table 7, with LNP 1 and LNP 2 used as controls. Mice were obtained from Jackson Laboratories and allowed to acclimate for one week prior to manipulations. For each LNMP, LPMP or LNP composition described in Table 7, mice were intramuscularly dosed with an experimental or control composition containing 1:1 Fluc:hEPO (5 ug each, 10 ug/40 uL dose, n=3/group). 4-6 hours post-dose, animals were injected with 200 μL of 15 mg/mL D-Luciferin (GoldBio), and placed in set nose cones inside the IVIS Lumina LT imager (PerkinElmer). LivingImage software was utilized for imaging. Whole body bio-luminescence was captured at auto-exposure after which animals were removed from the IVIS. This is displayed as average radiance. Mice were placed into a CO₂ chamber for euthanasia after. Cardiac puncture was performed on each animal after placing it in dorsal recumbency. Blood collection was performed using a 25G insulin syringe (BD). Once all blood samples were collected, tubes are spun at 2000G for 10 minutes using a tabletop centrifuge and plasma was aliquoted into individual Eppendorf tubes (Fisher Scientific) and stored at −80° C. for subsequent EPO quantification. EPO levels (pg/mL) in plasma were determined using EPO MSD kit (Meso Scale Diagnostics). Organs such as muscle, liver, spleen, popliteal lymph nodes (LNs), inguinal LNs, axillary LNs, brachial LNs, lungs, and marrow were collected and imaged as described above. Organs were imaged at 1 sec, 1 min, and 5 min exposures.

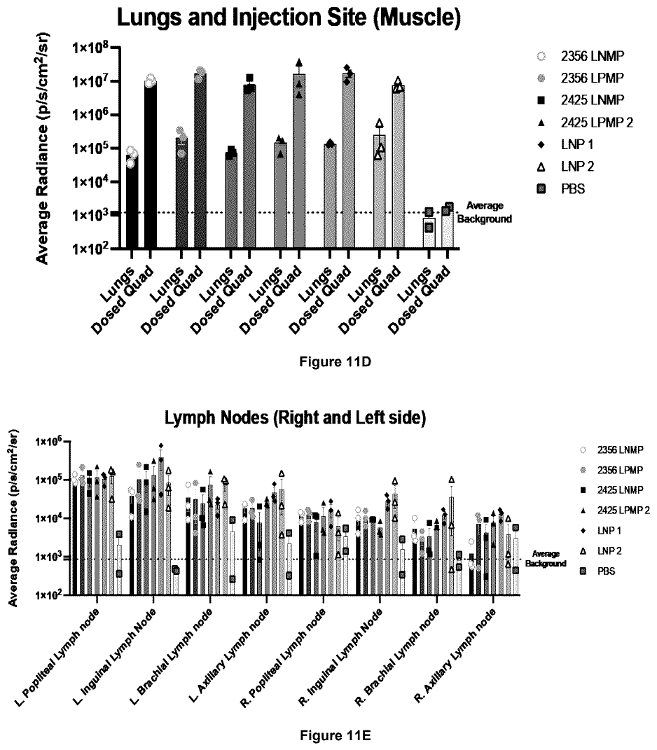

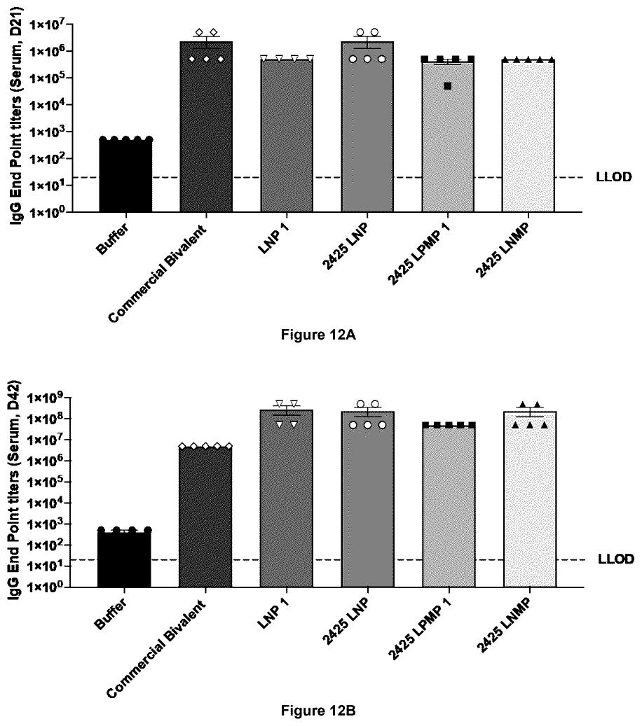

FIGS. 11A-11E show the whole body radiance (FIG. 11A), liver radiance (FIG. 11B), spleen radiance (FIG. 11C), lungs and quad muscle (injection site) radiance (FIG. 11D), and the right and left side lymph nodes radiance (FIG. 11E) at 4 hours post-dose of intramuscular administration of various exemplary LNMPs and LPMPs formulations employing the novel ionizable lipids (see Table 7) encapsulating 1:1 mRNA FLuc:hEPO, dosed at 10 ug/40 u. As shown in FIGS. 11A-11E, average radiance of the experimental groups, dosed with intramuscular administration of various exemplary LNMPs and LPMPs formulations employing the novel ionizable lipids disclosed herein exhibited lower liver expression than the control LNPs, indicating less systemic exposure, while exhibiting comparable expression levels at injection site (quad muscle) and in lymph nodes. The left side lymph nodes are on the same side as the injection site.

### Example 7: Testing of LNMP/SARS-CoV-2 Formulations in Mice

If not specified, the LNP/mRNA or LNMP/mRNA formulations were prepared according to those described in Example 1-3. The experiment was designed to test the immunogenicity of exemplary LNMP formulations in mice of the candidate coronavirus vaccines comprising an mRNA of Table 1 encoding a coronavirus antigen (e.g., the spike (S) protein), such as a SARS-CoV-2 antigen, as compared to a commercial vaccine. In this example, controls include commercial monovalent vaccines or bivalent vaccines that contain the S mRNA formulated within a conventional lipid nanoparticle composition (without natural lipids) containing either ALC-0315 ([(4-hydroxybutyl)azanediyl]di(hexane-6,1-diyl) bis(2-hexyldecanoate)) or SM-102 (heptadecan-9-yl 8-[2-hydroxyethyl-(6-oxo-6-undecoxyhexyl)amino]octanoate); a phospholipid; cholesterol; and a PEG-lipid. A commercial monovalent vaccine refers to the vaccine that contains the S mRNA targeting SARS-CoV-2 original strain; and a commercial bivalent vaccine refers to the vaccine that contains the S mRNA targeting SARS-CoV-2 original strain and Omicron BA.4/BA.5 strain.

### Formulation and Characterization of LNMP/mRNA

This example further describes the formulation of several LNMPs formulated with ionizable lipids, structural lipids (natural or natural combined with synthetic), sterols, and PEG lipids, to encapsulate mRNA (e.g., S mRNA) for LNMP/mRNA. LNMP compositions where the natural lipids were extracted from a plant source, i.e., lemon or soy in this example, were labeled as LPMP.

Lipid nanoparticle (LNP) formulation was prepared according to Example 1-3, composed of ionizable lipid:structural lipid:sterol:PEG-lipid at given molar ratios outlined in Table 8 or 10. Lipids were solubilized in ethanol. These lipids were mixed at the indicated molar ratios and diluted in ethanol (organic phase) to 5.5 mM total lipid concentration and the mRNA solution (aqueous phase) was prepared with RNAse-free water and 100 mM citrate buffer pH 3 for a final concentration of 50 mM citrate buffer. Formulations were maintained at ionizable lipid to mRNA N:P ratio of 3:1-15:1 (Table 8 or 10).

In some formulations, a lemon LPMP composition was formulated according to Example 1-3, composed of ionizable lipid:lemon lipid:sterol:PEG-lipid at given molar ratios outlined in Table 8 or 10. Lipids were solubilized in 4:1 DMF:ethanol. These lipids were mixed at the indicated molar ratios and diluted in ethanol (organic phase) to 5.5 mM total lipid concentration and the mRNA solution (aqueous phase) was prepared with RNAse-free water and 100 mM citrate buffer pH 3 for a final concentration of 50 mM citrate buffer. Formulations were maintained at ionizable lipid to mRNA N:P ratio of 3:1-15:1 (Table 8 or 10).

In some formulations, an E. coli LNMP composition was formulated according to Example 2, comprising ionizable lipid: E. coli polar lipids:sterol:PEG-lipid or ionizable lipid:(E. coli polar lipids+a neutral lipid):sterol:PEG-lipid at given molar ratios outlined in Table 8 or 10. The E. coli polar lipids were purchased from Avanti. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 3:1-15:1 (Table 8 or 10).

In some formulations, a soy LPMP composition was formulated according to Example 2, comprising ionizable lipid:soy lipids:sterol:PEG-lipid at given molar ratios outlined in Table 10. In some cases, the soy lipids were purchased from Avanti. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 3:1-15:1 (Table 10).

The lipid mix and mRNA solution were mixed at a 1:3 ratio by volume, respectively, on the NANOASSEMBLR® IGNITE™ (Precision Nanosystems) at a total flow rate of 14 mL/minute. The resulting formulations were then loaded into Slide-A-Lyzer G2 dialysis cassettes (10 k MWCO) and dialyzed against 1×PBS for 2 hours at room temperature. The PBS was refreshed, and the formulations were further dialyzed for at least 14 hours at 4° C. with gentle stirring. The dialyzed formulations were then collected and concentrated by centrifugation at 2000×g using AMICON® Ultra centrifugation filters (100 k MWCO). The concentrated formulations were characterized for size, polydispersity, and particle concentration using a Zetasizer Ultra (Malvern Panalytical) and for mRNA encapsulation efficiency using QUANT-IT™ RIBOGREEN® RNA Assay Kit (ThermoFisher Scientific).

TABLE 8
Characterizations of formulations

| | Ionizable:Structural: | | | | | | |
|---|---|---|---|---|---|---|---|
| | Sterol:PEG-lipid | | | | PEG- | N:P | |
| Formulation | Molar ratio | Ionizable | Structural | Sterol | lipid | Ratio | Cargo |
| LNP 1 | 50:10:38.5:1.5 | Lipid 8 | DSPC | Chol | DMG- | 6:1 | SARS-COV-2 |
| | | | | | PEG2k | | mRNA |
| 2425 LNP | 50:10:38.5:1.5 | 2425 | DSPC | Chol | DMG- | 6:1 | SARS-COV-2 |
| | | | | | PEG2k | | mRNA |
| 2425 LPMP 1 | 45:20:33.5:1.5 | 2425 | Naturally derived | Chol | DMG- | 12:1 | SARS-COV-2 |
| | | | lemon lipid | | PEG2k | | mRNA |
| 2425 LNMP | 45:20:33.5:1.5 | 2425 | E. coli polar lipids | Chol | DMG- | 12:1 | SARS-COV-2 |
| | | | | | PEG2k | | mRNA |

### Vaccination and Collection in Mice

8-9 week old C57BL/6 mice were utilized for vaccination efforts of the formulations provided in Table 8, with PBS, LNP 1, and a bivalent commercial vaccine acting as controls. Mice were obtained from Jackson Laboratories and allowed to acclimate for one week prior to manipulations. Five C57BL/6 mice were evaluated for each formulation, control, and buffer. The test samples were injected into mice on DO and D21 using the intramuscular (IM) route, in one upper thigh in the hind limb. On D21, nonterminal bleeds were collected. On D42, terminal bleeds were collected via retro-orbital puncture into a heparin blood collection tube. Samples were centrifuged for 2000 g for 10 minutes, and plasma was transferred and stored at −80° C. Spleens were collected as well and placed in a RPMI solution. Splenocytes were isolated on a cell strainer and filtered on 70 μm filters before and after red blood cell lysis. Antigen specific T cells were evaluated via ELISPOT as described below. Samples were transiently stored at −80° C. before being transferred to liquid nitrogen.

Blood plasma or serum collection. Blood samples were collected on D21 and D42, as described above and Examples 3-6.

Binding antibody titers evaluation by ELISA. The analysis of the antibody responses (IgG) against SARS-CoV-2 was performed to quantify the responses for mice antibodies targeting antigens of SARS-CoV-2 using an ELISA. IgG antibody titers against SARS-CoV-2 were determined by diluting plasma in 1/10 fold dilutions. Briefly, ELISA plates were coated with recombinant Spike RBD overnight. The following day, plates were blocked, samples were diluted, and all dilutions were added overnight. On day 3, detection antibodies and ELISA development were completed. Antibody titers were selected by choosing the last dilution where samples were no longer diluting.

ELISPOT cytokine evaluation of anti-SARS-CoV-2/S T cell responses. T cell responses against SARS-CoV-2 antigens were evaluated on D42 in mice using an IFNγ immunospot color ELISPOT assay. Splenocytes were isolated on a cell strainer and filtered on before and after red blood cell lysis. Live splenocytes were counted and their viability was assessed by acridine orange exclusion assay on a Luna cell counter. ELISPOT assays were run by stimulating the cells with SARS-CoV-2/S peptides pools overnight. The following day, the IFNγ producing cells were evaluated using the IFNγ color immunospot assay. The plates were analyzed using an Immunospot CTL-S6 Fluor analyzer.

Plaque Reduction Neutralization Test (PRNT). The SARS-CoV-2 PRNT assays were conducted on blood samples from D42. Vero E6 cells were plated in growth media (DMEM+10% FBS+gentamicin). The cells were incubated at 37° C., 5.0% CO2 until the cells reached 80-100% confluency the following day. Heat-inactivated serum samples or positive control virus in diluent media (DMEM+2% FBS+gentamicin) was added to the cells and mixed via pipetting at varying dilutions, then virus dilution was added to the sample and positive control wells. Plates were sealed and incubated for 1 h. Media was removed, titrated samples/virus dilutions were added in duplicate, and the plates were then incubated for 1 h again. Overlay methylcellulose media was then added and plates were incubated for 3 days. After infection, the methylcellulose overlay medium was removed, and the plates were washed and fixed. After fixation, the cell monolayers were stained with 0.2% crystal violet (30% MeOH, 80% dH2O) for 30 minutes at room temperature. The plates were washed and dried. Plaques in each well were recorded and the IC50 titers were calculated based on the average number of plaques detected in the virus control wells.

TABLE 9
Treatment details

| | Number | Dose | | |
|---|---|---|---|---|
| Particle ID | of animals | Route | Dose Conc/Vol | Payload |
| LNP 1 | 5 | IM | 5ug/40uL | S mRNA |
| 2425 LNP | 5 | | | |
| 2425 LPMP 1 | 5 | | | |
| 2425 LNMP | 5 | | | |
| Commercial | 5 | | | |
| Vaccine | | | | |
| Control | | | | |
| Buffer | 5 | | | |

In sum, five mice per experimental group were intramuscularly dosed with the LNMPs, buffer, or vaccine control as shown in Table 9 on Day 0 and Day 21 (5 ug/dose). Blood was drawn on Day 21, and blood and tissue was collected on Day 42. For this experiment, a commercially available bivalent vaccine was used in the first dose and in the second dose based on the dose level listed in Table 9.

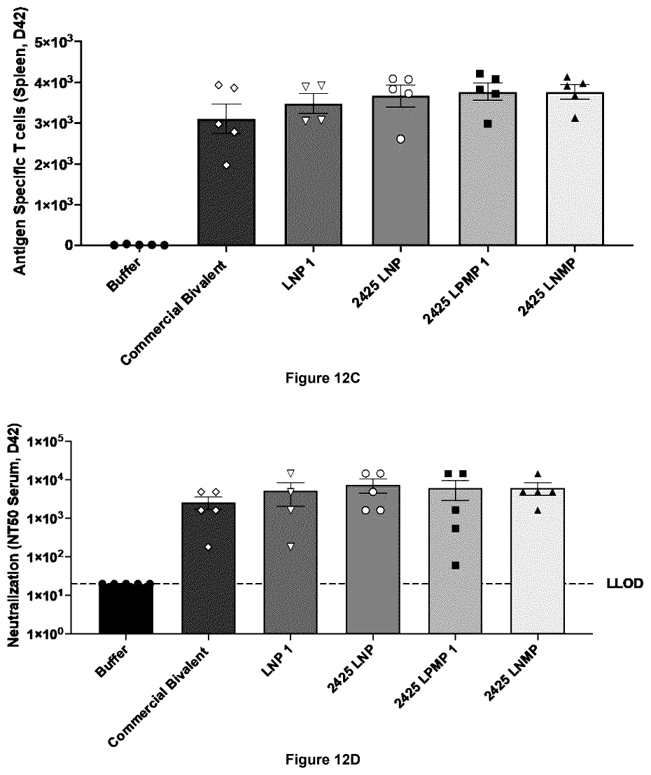

Antibody response (IgG) was evaluated by indirect ELISA at D21 and D42. FIGS. 12A and 12B show the endpoint titers of antibody (IgG) specific to the receptor binding domain (RBD) of SARS-CoV-2 in the blood of mice on D21 (FIG. 12A), after a single dose intramuscular delivery of the LNMP or LPMP formulations employing an exemplary novel ionizable lipid 2425 (S mRNA 5 μg, Table 8), and D42 (FIG. 12B), after a second dose intramuscular delivery of the LNMP or LPMP formulations employing an exemplary novel ionizable lipid 2425 (S mRNA 5 ug, Table 8). N=5/group. Controls were PBS, LNP 1, 2425 LNP, and a commercial bivalent vaccine. N=5/group. The results indicate comparable or better antibody responses in the LNMP or LPMP formulations employing an exemplary novel ionizable lipid 2425 as compared to the commercially available vaccine control, and as compared to the LNP1 and 2425 LNP controls.

Anti-SARS-CoV-2/S T cell responses were evaluated in corresponding ELISPOT assay (IFNγ) at D42. FIG. 12C shows the number of SARS-CoV-2 S-specific T cells producing cytokine IFNγ per 10⁶ splenocytes in mice at 42 days, after a prime and boost dose intramuscular delivery of the LNMP or LPMP formulations employing an exemplary novel ionizable lipid 2425 (S mRNA 5 μg, Table 8). N=5/group. Controls were PBS, LNP 1, 2425 LNP, and a commercial bivalent vaccine. N=5/group. The results indicate that the LNMP or LPMP formulations employing an exemplary novel ionizable lipid 2425 described in Table 8 induced high numbers of antigen-specific T cells in splenocytes than the commercially available vaccine control. Additionally, the LNMP and LPMP formulations employing an exemplary novel ionizable lipid 2425 also induced high numbers of antigen-specific T cells in splenocytes than the LNP1 and 2425 LNP controls.

Neutralization was analyzed at 42 days. FIG. 12D shows the levels of neutralizing antibody titer against the SARS-CoV-2 original strain in the blood of the mice at 42 days, after a prime and boost vaccination of the LNMP formulations employing an exemplary novel ionizable lipid 2425 (S mRNA 5 μg, Table 8). The results show that the LNMP and LPMP formulations employing an exemplary novel ionizable lipid 2425 had a neutralization higher than the commercially available vaccine after two intramuscular doses; and the LNMP and LPMP formulations employing an exemplary novel ionizable lipid 2425 also had a neutralization comparable to or higher than the LNP1 and 2425 LNP controls.

TABLE 10
Characterizations of formulations

| | Ionizable:Structural: | | | | | | |
|---|---|---|---|---|---|---|---|
| | Sterol:PEG-lipid | | | | PEG- | N:P | |
| Formulation | Molar ratio | Ionizable | Structural | Sterol | lipid | Ratio | Cargo |
| 2356 LNP | 35:16:46.5:2.5 | 2356 | DSPC | Chol | DMG- | 6:1 | SARS-COV-2 |
| | | | | | PEG2k | | mRNA |
| 2356 LPMP | 35:20:42.5:2.5 | 2356 | Soy PC | Chol | DMG- | 6:1 | SARS-COV-2 |
| | | | | | PEG2k | | mRNA |
| 2356 LNMP | 35:20:42.5:2.5 | 2356 | E. coli polar lipids | Chol | DMG- | 6:1 | SARS-COV-2 |
| | | | | | PEG2k | | mRNA |
| 2356 LPMP 1 | 35:20:42.5:2.5 | 2356 | Naturally derived | Chol | DMG- | 6:1 | SARS-COV-2 |
| | | | lemon lipid | | PEG2k | | mRNA |

### Vaccination and Collection in Mice

8-9 week old C57BL/6 mice were utilized for vaccination efforts of the formulations provided in Table 10, with PBS, LNP, and a bivalent commercial vaccine acting as controls. Mice were obtained from Jackson Laboratories and allowed to acclimate for one week prior to manipulations. Five C57BL/6 mice were evaluated for each formulation, control, and buffer. The test samples were injected into mice on DO and D21 using the intramuscular (IM) route, in one upper thigh in the hind limb. On D21, nonterminal bleeds were collected. On D42, terminal bleeds were collected via retro-orbital puncture into a heparin blood collection tube. Samples were centrifuged for 2000 g for 10 minutes, and plasma was transferred and stored at −80° C. Spleens were collected as well and placed in a RPMI solution. Splenocytes were isolated on a cell strainer and filtered on 70 μm filters before and after red blood cell lysis. Antigen specific T cells were evaluated via ELISPOT as described below. Samples were transiently stored at −80° C. before being transferred to liquid nitrogen.

Blood plasma or serum collection. Blood samples were collected on D21 and D42, as described above and Examples 3-6.

Binding antibody titers evaluation by ELISA. The analysis of the antibody responses (IgG) against SARS-CoV-2 was performed to quantify the responses for mice antibodies targeting antigens of SARS-CoV-2 using an ELISA. IgG antibody titers against SARS-CoV-2 were determined by diluting plasma in 1/10 fold dilutions. Briefly, ELISA plates were coated with recombinant Spike RBD overnight. The following day, plates were blocked, samples were diluted, and all dilutions were added overnight. On day 3, detection antibodies and ELISA development were completed. Antibody titers were selected by choosing the last dilution where samples were no longer diluting.

ELISPOT cytokine evaluation of anti-SARS-CoV-2/S T cell responses. T cell responses against SARS-CoV-2 antigens were evaluated on D42 in mice using an IFNγ immunospot color ELISPOT assay. Splenocytes were isolated on a cell strainer and filtered on before and after red blood cell lysis. Live splenocytes were counted and their viability was assessed by acridine orange exclusion assay on a Luna cell counter. ELISPOT assays were run by stimulating the cells with SARS-CoV-2/S peptides pools overnight. The following day, the IFNγ producing cells were evaluated using the IFNγ color immunospot assay. The plates were analyzed using an Immunospot CTL-S6 Fluor analyzer.

Plaque Reduction Neutralization Test (PRNT). The SARS-CoV-2 PRNT assays were conducted on blood samples from D42. Vero E6 cells were plated in growth media (DMEM+10% FBS+gentamicin). The cells were incubated at 37° C., 5.0% CO2 until the cells reached 80-100% confluency the following day. Heat-inactivated serum samples or positive control virus in diluent media (DMEM+2% FBS+gentamicin) was added to the cells and mixed via pipetting at varying dilutions, then virus dilution was added to the sample and positive control wells. Plates were sealed and incubated for 1 h. Media was removed, titrated samples/virus dilutions were added in duplicate, and the plates were then incubated for 1 h again. Overlay methylcellulose media was then added and plates were incubated for 3 days. After infection, the methylcellulose overlay medium was removed, and the plates were washed and fixed. After fixation, the cell monolayers were stained with 0.2% crystal violet (30% MeOH, 80% dH2O) for 30 minutes at room temperature. The plates were washed and dried. Plaques in each well were recorded and the IC50 titers were calculated based on the average number of plaques detected in the virus control wells.

TABLE 11
Treatment details

| | Number | Dose | | |
|---|---|---|---|---|
| Particle ID | of animals | Route | Dose Conc/Vol | Payload |
| 2356 LNP | 5 | IM | 5ug/40uL | S mRNA |
| 2356 LPMP | 5 | | | |
| 2356 LNMP | 5 | | | |
| 2356 LPMP 1 | 5 | | | |
| Commercial | 5 | | | |
| Vaccine | | | | |
| Control | | | | |
| Buffer | 5 | | | |

In sum, five mice per experimental group were intramuscularly dosed with the LNMPs, buffer, or vaccine control as shown in Table 11 on Day 0 and Day 21 (5 ug/dose). Blood was drawn on Day 21, and blood and tissue was collected on Day 42. For this experiment, a commercially available bivalent vaccine was used in the first dose and in the second dose based on the dose level listed in Table 11.

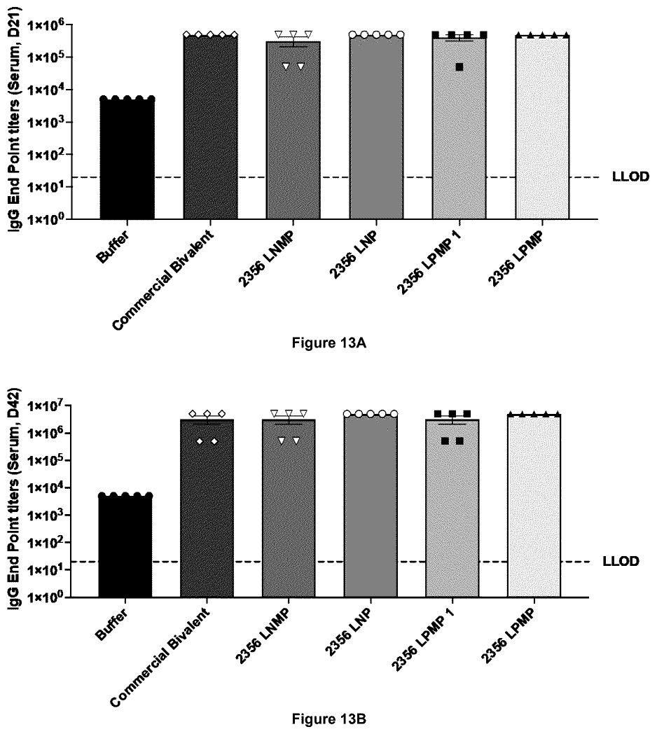

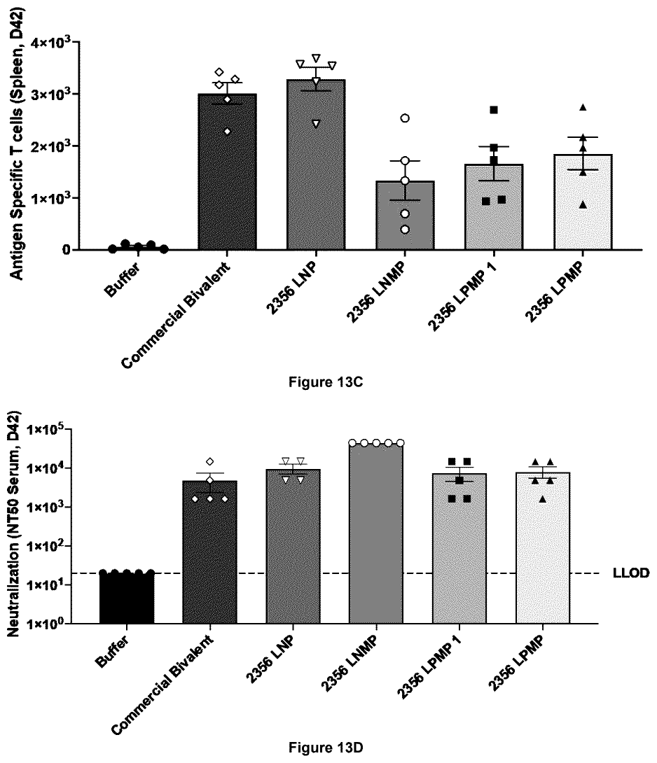

Antibody response (IgG) was evaluated by indirect ELISA at D21 and D42. FIGS. 13A and 13B shows the endpoint titers of antibody (IgG) specific to the receptor binding domain (RBD) of SARS-CoV-2 in the blood of mice on D21 (FIG. 13A), after a single dose intramuscular delivery of the LNMP or LPMP formulations (S mRNA 5 μg, Table 10), and D42 (FIG. 13B), after a second dose intramuscular delivery of the LNMP or LPMP formulations employing an exemplary novel ionizable lipid 2356 (S mRNA 5 ug, Table 10). N=5/group. Controls were PBS, 2356 LNP, and a commercial bivalent vaccine. N=5/group. The results indicate comparable or better antibody responses in the LNMP and LPMP formulations employing an exemplary novel ionizable lipid 2356 as compared to the commercially available vaccine control, and as compared to the 2356 LNP control.

Anti-SARS-CoV-2/S T cell responses were evaluated in corresponding ELISPOT assay (IFNγ) at D42. FIG. 13C shows the number of SARS-CoV-2 S-specific T cells producing cytokine IFNγ per 10⁶ splenocytes in mice at 42 days, after a prime and boost dose intramuscular delivery of the LNMP or LPMP formulations employing an exemplary novel ionizable lipid 2356 (S mRNA 5 μg, Table 10). N=5/group. Controls were PBS, 2356 LNP, and a commercial bivalent vaccine. N=5/group. The results indicate that the LNMP and LPMP formulations employing an exemplary novel ionizable lipid 2356 described in Table 10 induced high numbers of antigen-specific T cells in splenocytes, though lower than the commercially available vaccine control.

Neutralization was analyzed at 42 days. FIG. 13D shows the levels of neutralizing antibody titer against the SARS-CoV-2 original strain in the blood of the mice at 42 days, after a prime and boost vaccination of the LNMP formulations employing an exemplary novel ionizable lipid 2356 (S mRNA 5 μg, Table 10). The results show that all the LNMP and LPMP formulations employing an exemplary novel ionizable lipid 2356 had a neutralization higher than the commercially available vaccine after two intramuscular doses; and higher than or comparable to the 2356 LNP control.

### Example 8: Dosing Variations in Mice

If not specified, the LNP/mRNA or LNMP/mRNA formulations were prepared according to those described in Example 1-3. The experiment was designed to test dosing of exemplary LNMP formulations in mice of the candidate coronavirus vaccines comprising an mRNA of Table 1 encoding a coronavirus antigen (e.g., the spike (S) protein), such as a SARS-CoV-2 antigen. In this example, controls include commercial monovalent vaccines or bivalent vaccines that contain the S mRNA formulated within a conventional lipid nanoparticle composition (without natural lipids) containing either ALC-0315 ([(4-hydroxybutyl)azanediyl]di(hexane-6,1-diyl) bis(2-hexyldecanoate)) or SM-102 (heptadecan-9-yl 8-[2-hydroxyethyl-(6-oxo-6-undecoxyhexyl)amino]octanoate); a phospholipid; cholesterol; and a PEG-lipid. A commercial monovalent vaccine refers to the vaccine that contains the S mRNA targeting SARS-CoV-2 original strain; and a commercial bivalent vaccine refers to the vaccine that contains the S mRNA targeting SARS-CoV-2 original strain and Omicron BA.4/BA.5 strain.

### Formulation and Characterization of LNMP/mRNA

This example further describes the formulation of several LNMPs formulated with ionizable lipids, structural lipids (natural or natural combined with synthetic), sterols, and PEG lipids, to encapsulate mRNA (e.g., S mRNA) for LNMP/mRNA. LNMP compositions where the natural lipids were extracted from a plant source, i.e., lemon or soy in this example, were labeled as LPMP.

Lipid nanoparticle (LNP) formulation was prepared according to Example 1-3, composed of ionizable lipid:structural lipid:sterol:PEG-lipid at given molar ratios outlined in Table 12. Lipids were solubilized in ethanol. These lipids were mixed at the indicated molar ratios and diluted in ethanol (organic phase) to 5.5 mM total lipid concentration and the mRNA solution (aqueous phase) was prepared with RNAse-free water and 100 mM citrate buffer pH 3 for a final concentration of 50 mM citrate buffer. Formulations were maintained at ionizable lipid to mRNA N:P ratio of 3:1-15:1 (Table 12).

In some formulations, a lemon LPMP composition was formulated according to Example 1-3, composed of ionizable lipid:lemon lipid:sterol:PEG-lipid at given molar ratios outlined in Table 12. Lipids were solubilized in 4:1 DMF:ethanol. These lipids were mixed at the indicated molar ratios and diluted in ethanol (organic phase) to 5.5 mM total lipid concentration and the mRNA solution (aqueous phase) was prepared with RNAse-free water and 100 mM citrate buffer pH 3 for a final concentration of 50 mM citrate buffer. Formulations were maintained at ionizable lipid to mRNA N:P ratio of 3:1-15:1 (Table 12).

In some formulations, an E. coli LNMP composition was formulated according to Example 2, comprising ionizable lipid: E. coli polar lipids:sterol:PEG-lipid or ionizable lipid:(E. coli polar lipids+a neutral lipid):sterol:PEG-lipid at given molar ratios outlined in Table 12. The E. coli polar lipids were purchased from Avanti. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 3:1-15:1 (Table 12).

In some formulations, a soy LPMP composition was formulated according to Example 2, comprising ionizable lipid:soy lipids:sterol:PEG-lipid at given molar ratios outlined in Table 12. In some cases, the soy lipids were purchased from Avanti. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 3:1-15:1 (Table 12).

The lipid mix and mRNA solution were mixed at a 1:3 ratio by volume, respectively, on the NANOASSEMBLR® IGNITE™ (Precision Nanosystems) at a total flow rate of 14 mL/minute. The resulting formulations were then loaded into Slide-A-Lyzer G2 dialysis cassettes (10 k MWCO) and dialyzed against 1×PBS for 2 hours at room temperature. The PBS was refreshed, and the formulations were further dialyzed for at least 14 hours at 4° C. with gentle stirring. The dialyzed formulations were then collected and concentrated by centrifugation at 2000×g using AMICON® Ultra centrifugation filters (100 k MWCO). The concentrated formulations were characterized for size, polydispersity, and particle concentration using a Zetasizer Ultra (Malvern Panalytical) and for mRNA encapsulation efficiency using QUANT-IT™ RIBOGREEN® RNA Assay Kit (ThermoFisher Scientific).

TABLE 12
Characterizations of formulations

| | Ionizable:Structural: | | | | | | |
|---|---|---|---|---|---|---|---|
| | Sterol:PEG-lipid | | | | PEG- | N:P | |
| Formulation | Molar ratio | Ionizable | Structural | Sterol | lipid | Ratio | Cargo |
| LNP 1 | 50:10:38.5:1.5 | Lipid 8 | DSPC | Chol | DMG- | 6:1 | SARS-COV-2 |
| | | | | | PEG2k | | mRNA |
| 2425 LNP | 50:10:38.5:1.5 | 2425 | DSPC | Chol | DMG- | 12:1 | SARS-COV-2 |
| | | | | | PEG2k | | mRNA |
| 2425 LPMP 1 | 45:20:33.5:1.5 | 2425 | Naturally derived | Chol | DMG- | 12:1 | SARS-COV-2 |
| | | | lemon lipid | | PEG2k | | mRNA |
| 2425 LPMP 2 | 45:20:33.5:1.5 | 2425 | Soy PC | Chol | DMG- | 12:1 | SARS-COV-2 |
| | | | | | PEG2k | | mRNA |
| 2425 LNMP | 45:20:33.5:1.5 | 2425 | E. coli polar lipids | Chol | DMG- | 12:1 | SARS-COV-2 |
| | | | | | PEG2k | | mRNA |

### Vaccination and Collection in Mice

8-9 week old C57BL/6 mice were utilized for vaccination efforts of the formulations provided in Table 12, with PBS, LNP 1, and a bivalent commercial vaccine acting as controls. Mice were obtained from Jackson Laboratories and allowed to acclimate for one week prior to manipulations. Five C57BL/6 mice were evaluated for each formulation at three different doses. The test samples were injected into mice on DO using the intramuscular (IM) route, in one upper thigh in the hind limb. On D14, nonterminal bleeds were collected. On D28, terminal bleeds were collected via retro-orbital puncture into a heparin blood collection tube. Samples were centrifuged for 2000 g for 10 minutes, and plasma was transferred and stored at −80° C. Spleens were collected as well and placed in a RPMI solution. Splenocytes were isolated on a cell strainer and filtered on 70 μm filters before and after red blood cell lysis. Antigen specific T cells were evaluated via ELISPOT as described below. Samples were transiently stored at −80° C. before being transferred to liquid nitrogen.

Blood plasma or serum collection. Blood samples were collected, as described above and Examples 3-6.

Binding antibody titers evaluation by ELISA. The analysis of the antibody responses (IgG) against SARS-CoV-2 was performed to quantify the responses for mice antibodies targeting antigens of SARS-CoV-2 using an ELISA. IgG antibody titers against SARS-CoV-2 were determined by diluting plasma in 1/10 fold dilutions. Briefly, ELISA plates were coated with recombinant Spike RBD overnight. The following day, plates were blocked, samples were diluted, and all dilutions were added overnight. On day 3, detection antibodies and ELISA development were completed. Antibody titers were selected by choosing the last dilution where samples were no longer diluting.

ELISPOT cytokine evaluation of anti-SARS-CoV-2/S T cell responses. T cell responses against SARS-CoV-2 antigens were evaluated in mice using an IFNγ immunospot color ELISPOT assay. Splenocytes were isolated on a cell strainer and filtered on before and after red blood cell lysis. Live splenocytes were counted and their viability was assessed by acridine orange exclusion assay on a Luna cell counter. ELISPOT assays were run by stimulating the cells with SARS-CoV-2/S peptides pools overnight. The following day, the IFNγ producing cells were evaluated using the IFNγ color immunospot assay. The plates were analyzed using an Immunospot CTL-S6 Fluor analyzer.

TABLE 13
Treatment details

| | Number | | | |
|---|---|---|---|---|
| Particle ID | of animals | Dose Route | Dose Conc/Vol | Payload |
| LNP 1 | 5 | IM | 0.4ug/40uL, | S mRNA |
| 2425 LNP | 5 | | 1ug/40uL, | |
| 2425 LPMP 1 | 5 | | 10ug/40uL | |
| 2425 LPMP 2 | 5 | | | |
| 2425 LNMP | 5 | | | |
| Commercial | 5 | | | |
| Vaccine | | | | |
| Buffer | 5 | | | |

In sum, five mice per experimental group were intramuscularly dosed with the LNMPs, LNPs, buffer, or a commercial bivalent vaccine as shown in Table 13 on Day 0 (0.4 ug, 1 ug, or 10 ug/dose). Blood was drawn on Day 14, and blood and tissue was collected on Day 28. For this experiment, a commercially available bivalent vaccine was used based on the dose level listed in Table 13.

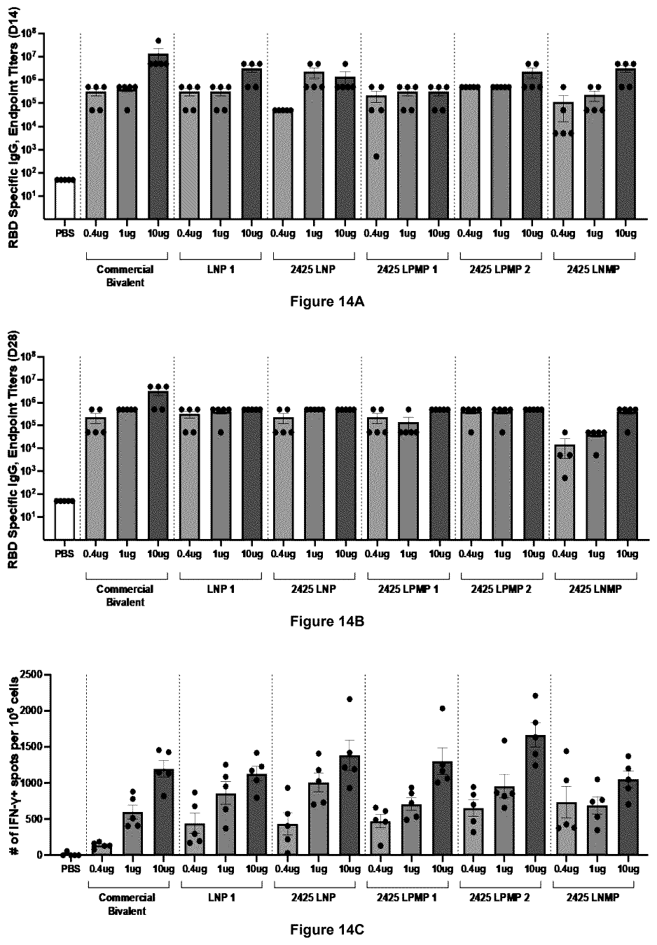

Antibody response (IgG) was evaluated by indirect ELISA at D14 and D28. FIGS. 14A and 14B show the endpoint titers of antibody (IgG) specific to the receptor binding domain (RBD) of SARS-CoV-2 in the blood of mice on D14 (FIG. 14A) and D28 (FIG. 14B), after a single dose intramuscular delivery of the LNMP or LPMP formulations employing an exemplary novel ionizable lipid 2425 (S mRNA 0.4, 1, or 10 μg, Table 12). N=5/group. Controls were PBS, LNP 1, 2425 LNP, and a commercial bivalent vaccine. N=5/group. The results show dose dependency and indicate comparable or higher than antibody responses in the LNMP or LPMP formulations employing an exemplary novel ionizable lipid 2425 as compared to the commercially available vaccine control, and compared to the LNP1 and 2425 LNP controls, particularly at 1 ug and 10 ug.

Anti-SARS-CoV-2/S T cell responses were evaluated in corresponding ELISPOT assay (IFNγ) at D28. FIG. 14C shows the number of SARS-CoV-2 S-specific T cells producing cytokine IFNγ per 10⁶ splenocytes in mice at 28 days, after a single dose intramuscular delivery of the LNMP or LPMP formulations employing an exemplary novel ionizable lipid 2425 (S mRNA 0.4, 1, or 10 μg, Table 12). N=5/group. Controls were PBS, LNP 1, 2425 LNP, and a commercial bivalent vaccine. N=5/group. The results indicate that the LNMP or LPMP formulations employing an exemplary novel ionizable lipid 2425 described in Table 12 had a dose dependent T cell response in the spleen. In general, at each dose level, the LNMP or LPMP formulations employing an exemplary novel ionizable lipid 2425 induced a number of antigen-specific T cells in splenocytes higher than or comparable to the commercially available vaccine control. At each dose level, the LNMP and LPMP formulations employing an exemplary novel ionizable lipid 2425 also induced a number of antigen-specific T cells in splenocytes higher than or comparable to the LNP1 and 2425 LNP controls.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference. Other embodiments are within the claims.

1. A nucleic acid vaccine, comprising:
one or more polynucleotides encoding one or more antigenic polypeptides derived from an infectious agent that causes an infectious disease, disorder, or condition,
formulated within a lipid reconstructed natural messenger packs (LNMPs) comprising natural lipids and an ionizable lipid, wherein the ionizable lipid has two or more of the characteristics listed below:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heterorganic group separated by a chain of at least two atoms; and
(v) an N:P ratio of at least 3.

2. A nucleic acid vaccine, comprising:
one or more polynucleotides encoding one or more antigenic polypeptides derived from an infectious agent that causes an infectious disease, disorder, or condition,
formulated within a lipid reconstructed natural messenger packs (LNMPs) comprising natural lipids and an ionizable lipid, wherein the ionizable lipid is selected from one of the following groups of compounds:
i) a compound of formula 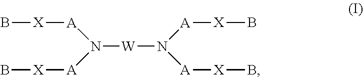
a pharmaceutically acceptable salt thereof, or a stereoisomer of any of the foregoing, wherein:
each A is independently C₁-C₁₆ branched or unbranched alkyl or C₁-C₁₆ branched or unbranched alkenyl, optionally substituted with heteroatom or substituted with OH, SH, or halogen;
each B is independently C₁-C₁₆ branched or unbranched alkyl or C₁-C₁₆ branched or unbranched alkenyl, optionally substituted with heteroatom or substituted with OH, SH, or halogen;
each X is independently a biodegradable moiety; and
W is 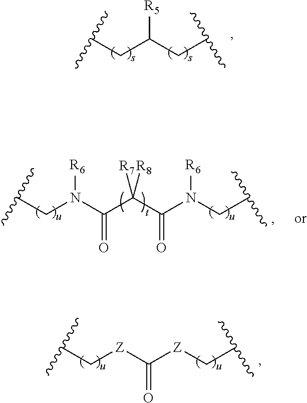
wherein:
R₅ is OH, SH, NR₁₀R₁₁;
each R₆ is independently H, C₁-C₃ branched or unbranched alkyl, C₂-C₃ branched or unbranched alkenyl, or cycloalkyl;
each R₇ and each R₈ is independently H, C₁-C₃ branched or unbranched alkyl, C₂-C₃ branched or unbranched alkenyl, halogen, OH, SH, or NR₁₀R₁₁, wherein each R₁₀ and R₁₁ is independently H, C₁-C₃ alkyl, or R₁₀ and Rn are taken together to form a heterocyclic ring; R₇ and R₈ are taken together to form a ring;
each s is independently 1, 2, 3, 4, or 5;
each u is independently 1, 2, 3, 4, or 5;
t is 1, 2, 3, 4 or 5;
each Z is independently absent, O, S, or NR₁₂, wherein R₁₂ is H, C₁-C₇ branched or unbranched alkyl, or C₂-C₇ branched or unbranched alkenyl, and
Q is O, S, or NR₁₃, wherein each R₁₃ is H, or C₁-C₅ alkyl;
ii) a compound of formula 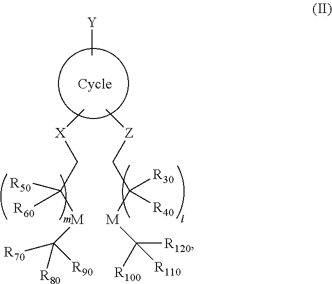
a pharmaceutically acceptable salt thereof, or a stereoisomer of any of the foregoing, wherein: 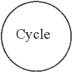
is a cyclic or heterocyclic moiety;
Y is alkyl, hydroxy, hydroxyalkyl or 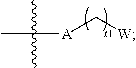
A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each of X and Z is independently absent, —O—, —CO—, —N(R⁷)—, —O-alkylene-, -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, hydroxyalkyl, or aminoalkyl;
each M is independently a biodegradable moiety;
each of R₃, R₄₀, R₅₀, R₆₀, R₇₀, R₈₀, R₉₀, R₁₀₀, R₁₁₀, and R₁₂₀ is independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
each of l and m is an integer from 1 to 10;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl; and
iii) a compound of formula 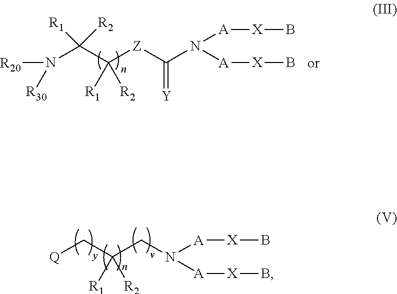
pharmaceutically acceptable salts thereof, and stereoisomers of any of the foregoing, wherein:
R₂₀ and R₃₀ are each independently H, C₁-C₅ branched or unbranched alkyl, or C₂-C₅ branched or unbranched alkenyl, or R₂₀ and R₃₀ together with the adjacent N atom form a 3 to 7 membered cyclic ring, optionally substituted with R^{a};
R^{a} is H, C₁-C₃ branched or unbranched alkyl, C₂-C₃ branched or unbranched alkenyl, halogen, OH, or SH;
each R₁ and each R₂ is independently H, C₁-C₃ branched or unbranched alkyl, C₂-C₃ branched or unbranched alkenyl, OH, halogen, SH, or NR₁₀R₁₁, or
R₁ and R₂ are taken together to form a cyclic ring;
each R₁₀ and R₁₁ is independently H, C₁-C₃ branched or unbranched alkyl, C₂-C₃ branched or unbranched alkenyl, or R₁₀ and R₁₁ are taken together to form a heterocyclic ring;
n is 0, 1, 2, 3 or 4;
Y is O or S;
Z is absent, O, S, or N(R₁₂), wherein each R₁₂ is independently H, C₁-C₇ branched or unbranched alkyl, or C₂-C₇ branched or unbranched alkenyl, provided that when Z is not absent, the adjacent R₁ and R₂ cannot be OH, NR₁₀R₁₁, or SH;
v is 0, 1, 2, 3, or 4;
y is 0, 1, 2, 3, or 4;
each A is each independently C₁-C₁₆ branched or unbranched alkyl, or C₂-C₁₆ branched or unbranched alkenyl, optionally interrupted with one or more heteroatoms or optionally substituted with OH, SH, or halogen;
each B is each independently C₁-C₁₆ branched or unbranched alkyl, or C₂-C₁₆ branched or unbranched alkenyl, optionally interrupted with one or more heteroatoms or optionally substituted with OH, SH, or halogen; and
each X is independently a biodegradable moiety; and
iv) a lipid comprising at least one head group and at least one tail group of formula (TI) or (TI′) 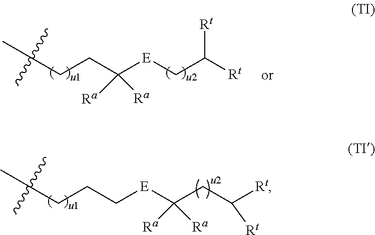
a pharmaceutically acceptable salt thereof, or a stereoisomer of any of the foregoing,
wherein:
E is each independently —OC(O)—, —C(O)O—, —N(R⁷)C(O)—, —C(O)N(R⁷)—, —C(O—R₁₃)—O—, —C(O)O(CH₂)ᵣ—, —C(O)N(R⁷)(CH₂)ᵣ—, —S—S—, or —C(O—R₁₃)—O—(CH₂)ᵣ—, wherein each R⁷ is independently H, alkyl, alkenyl, cycloalkyl, hydroxyalkyl, or aminoalkyl;
R₁₃ is branched or unbranched C₃-C₁₀ alkyl;
r is 1, 2, 3, 4, or 5;
R^{a} is each independently C₁-C₅ alkyl, C₂-C₅ alkenyl, or C₂-C₅ alkynyl;
u1 and u2 are each independently 0, 1, 2, 3, 4, 5, 6, or 7;
R^{t} is each independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl; [symbol]
represents the bond connecting the tail group to the head group; and
wherein the lipid has a pKa from about 4 to about 8.

3. The nucleic acid vaccine of claim 2, wherein the ionizable lipid is a compound of group i), represented by a formula of 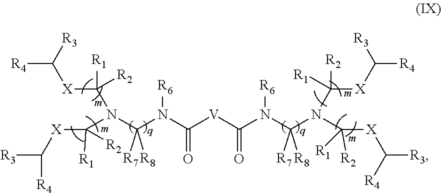
pharmaceutically acceptable salts thereof, and stereoisomers of any of the foregoing, wherein:
each R₁ and each R₂ is independently H, C₁-C₃ branched or unbranched alkyl, OH, halogen, SH, or NR₁₀R₁₁, or
each R₁ and each R₂ are independently taken together with the carbon atom(s) to which they are attached to form a cyclic ring;
each R₁₀ and R₁₁ is independently H, C₁-C₃ branched or unbranched alkyl, or R₁₀ and Rn are taken together to form a heterocyclic ring;
each R₃ and each R₄ is independently H, C₂-C₁₄ branched or unbranched alkyl (e.g., C₃-C₁₀ branched or unbranched alkyl), or C₃-C₁₀ branched or unbranched alkenyl, provided that at least one of R₃ and R₄ is not H;
each X is independently a biodegradable moiety;
each q is independently 2, 3, 4, or 5;
V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups;
each R₆ is independently H, C₁-C₃ branched or unbranched alkyl, C₂-C₃ branched or unbranched alkenyl, or cycloalkyl;
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein each v is independently 0, 1, 2, 3, 4, or 5, and R₁₇ is OH, SH, or N(CH₃)₂, and
each m is independently 1, 2, 3, 4, 5, 6, 7, 8, 9 or 10.

4. (canceled)

5. The nucleic acid vaccine of claim 2, wherein the ionizable lipid is a compound of group i), represented by a formula of 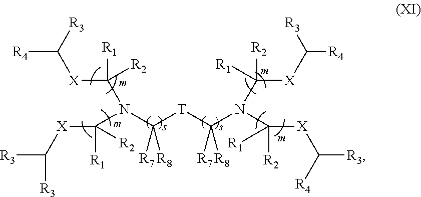
pharmaceutically acceptable salts thereof, and stereoisomers of any of the foregoing, wherein:
each R₁ and each R₂ is independently H, C₁-C₃ branched or unbranched alkyl, OH, halogen, SH, or NR₁₀R₁₁, or
each R₁ and each R₂ are independently taken together with the carbon atom(s) to which they are attached to form a cyclic ring;
each R₁₀ and R₁₁ is independently H, C₁-C₃ branched or unbranched alkyl, or R₁₀ and R₁₁ are taken together to form a heterocyclic ring;
each R₃ and each R₄ is independently H, C₂-C₁₄ branched or unbranched alkyl (e.g., C₃-C₁₀ branched or unbranched alkyl), or C₃-C₁₀ branched or unbranched alkenyl, provided that at least one of R₃ and R₄ is not H;
each X is independently a biodegradable moiety;
each s is independently 1, 2, 3, 4, or 5;
T is —NHC(O)O—, —OC(O)NH—, or a divalent heterocyclic optionally substituted with one or more —(CH₂)ᵥOH, —(CH₂)ᵥSH, or —(CH₂)ᵥ-halogen groups,
each R₇ and each R₈ is independently H, C₁-C₃ branched or unbranched alkyl, C₂-C₃ branched or unbranched alkenyl, halogen, OH, SH, (CH₂)ᵥR₁₇, or NR₁₀R₁₁, wherein R₁₇ is OH, SH, or N(CH₃)₂;
each v is independently 0, 1, 2, 3, 4, or 5; and
each m is independently 1, 2, 3, 4, 5, 6, 7, 8, 9 or 10.

6-7. (canceled)

8. The nucleic acid vaccine of claim 2, wherein the ionizable lipid is a compound of group ii), represented by one of the following formulas: 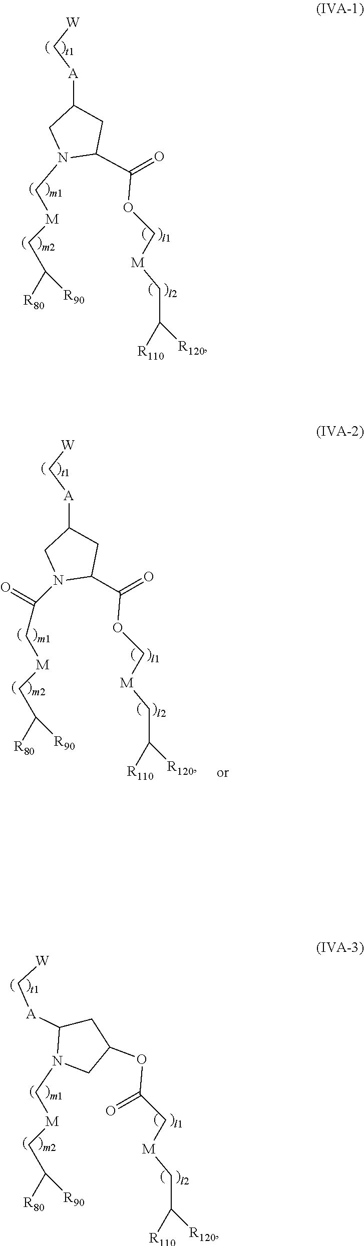
wherein:
A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each R⁷ is independently H, alkyl, alkenyl, cycloalkyl, hydroxy, hydroxyalkyl, or aminoalkyl;
t1 is an integer from 0 to 10;
W is hydroxyl, substituted or unsubstituted hydroxyalkyl, substituted or unsubstituted amino, substituted or unsubstituted aminocarbonyl, or substituted or unsubstituted heterocyclyl or heteroaryl;
each M is independently a biodegradable moiety;
each m1 is independently an integer from 3 to 6,
each l1 is independently an integer from 4 to 8,
m2 and l2 are each independently an integer from 0 to 3,
R₈₀ and R₉₀ are each independently unsubstituted C₅-C₈ alkyl or alkenyl; or R₈₀ is H or unsubstituted C₁-C₄ alkyl or alkenyl, and R₉₀ is unsubstituted C₅-C₁₁ alkyl or alkenyl; and
R₁₁₀ and R₁₂₀ are each independently unsubstituted C₅-C₈ alkyl or alkenyl; or R₁₁₀ is H or unsubstituted C₁-C₄ alkyl or alkenyl, and R₁₂₀ is unsubstituted C₅-C₁₁ alkyl or alkenyl.

9. The nucleic acid vaccine of claim 8, wherein:
M is —OC(O)— or —C(O)O—; 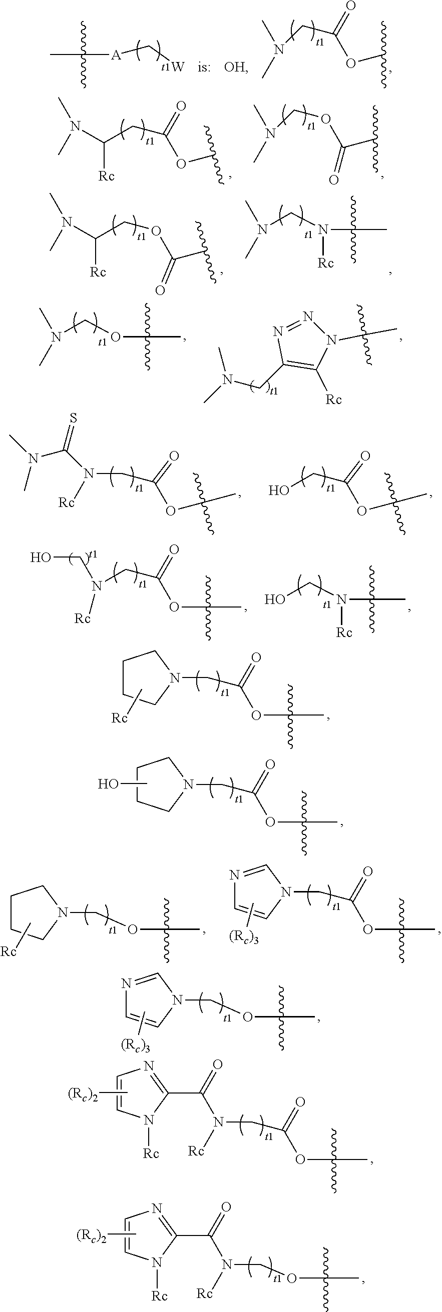 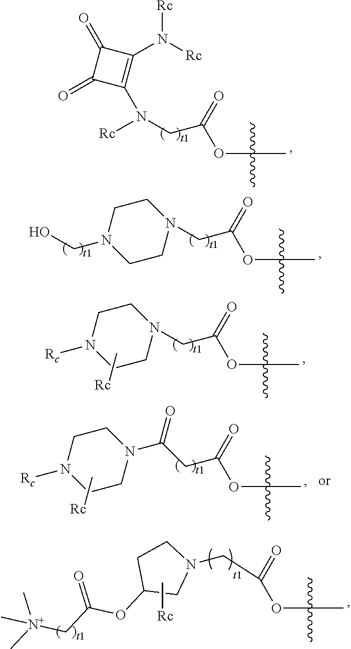
each R^{c} is independently H or C₁-C₃ alkyl; and
each t1 is independently 1, 2, 3, or 4.

10. The nucleic acid vaccine of claim 2, wherein the ionizable lipid is a compound of group iii), wherein
R₁ and R₂ are each H, or each R₁ is H, and one of the R₂ variables is OH;
X is —OC(O)— or —C(O)O—;
R₂₀ and R₃₀ are each independently H or C₁-C₃ branched or unbranched alkyl; or R₂₀ and R₃₀ together with the adjacent N atom form a 3 to 7 membered cyclic ring, optionally substituted with R^{a};
R^{a} is H or OH;
Z is absent, S, O, or NH; and
n is 0, 1, or 2.

11-12. (canceled)

13. The nucleic acid vaccine of claim 2, wherein the ionizable lipid is a compound of group iv), comprising at least one head group and at least one tail group, wherein:
the tail group has a structure of formula (TI) (or TI′); and
the head group has a structure of one of the following formulas: 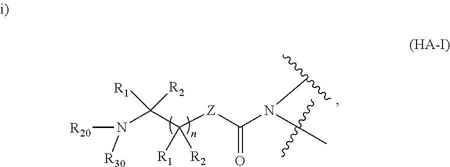
wherein:
R₂₀ and R₃₀ are each independently H, C₁-C₅ branched or unbranched alkyl, or C₂-C₅ branched or unbranched alkenyl, optionally interrupted with one or more heteroatoms or substituted with OH, SH, halogen, or cycloalkyl groups; or
R₂₀ and R₃₀, together with the adjacent N atom, form a 3 to 7 membered heterocyclic or heteroaromatic ring containing one or more heteroatoms, optionally substituted with one or more OH, SH, halogen, alkyl, or cycloalkyl groups;
each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ together form a cyclic ring;
each of R₁₀ and R₁₁ is independently H, C₁-C₃ branched or unbranched alkyl, C₂-C₃ branched or unbranched alkenyl; or R₁₀ and R₁₁ together form a heterocyclic ring;
n is 0, 1, 2, 3 or 4; and
Z is absent, O, S, or NR₁₂, wherein R₁₂ is H or C₁-C₇ branched or unbranched alkyl; provided that when Z is not absent, the adjacent R₁ and R₂ cannot be OH, NR₁₀R₁₁, or SH; 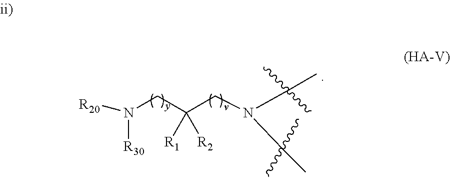
wherein:
R₁ is H, C₁-C₃ alkyl, OH, halogen, SH, or NR₁₀R₁₁;
R₂ is OH, halogen, SH, or NR₁₀R₁₁; or R₁ and R₂ can be taken together to form a cyclic ring;
R₁₀ and R₁₁ are each independently H or C₁-C₃ alkyl; or R₁₀ and R₁₁ can be taken together to form a heterocyclic ring;
R₂₀ and R₃₀ are each independently H, C₁-C₅ branched or unbranched alkyl, C₂-C₅ branched or unbranched alkenyl; or R₂₀ and R₃₀ can be taken together to form a cyclic ring; and
each of v and y is independently 1, 2, 3, or 4; 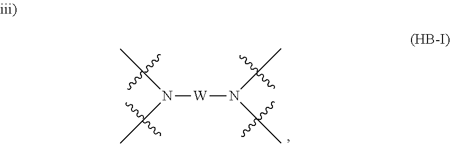
wherein W is 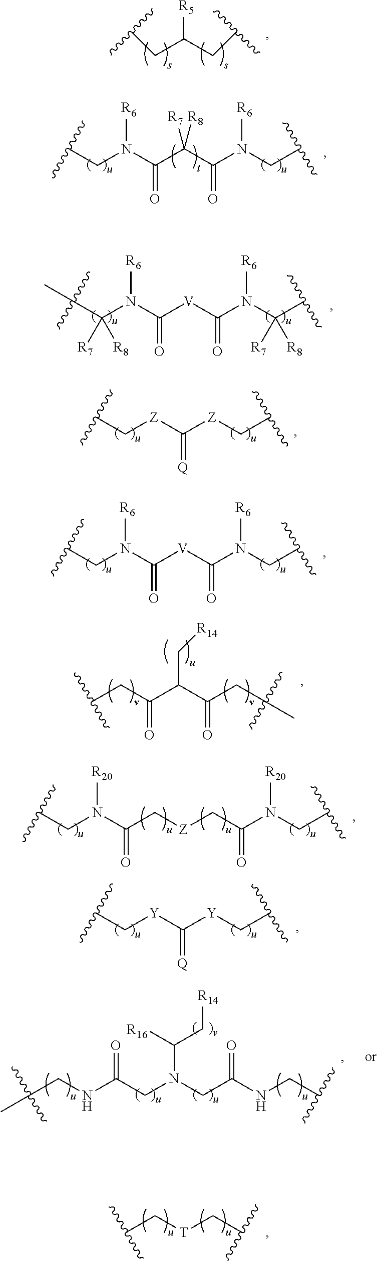
wherein
R₅ is OH, SH, (CH₂)ₛOH, or NR₁₀R₁₁;
each R₆ is independently H, C₁-C₃ branched or unbranched alkyl, C₂-C₃ branched or unbranched alkenyl, or cycloalkyl;
each R₇ and R₈ are independently H, C₁-C₃ branched or unbranched alkyl, C₂-C₃ branched or unbranched alkenyl, halogen, (CH₂)ᵥOH, (CH₂)ᵥSH, (CH₂)ₛN(CH₃)₂, or NR₁₀R₁₁, wherein each R₁₀ and R₁₁ is independently H or C₁-C₃ alkyl, or R₁₀ and R₁₁ are taken together to form a heterocyclic ring; or R₇ and R₈ are taken together to form a ring;
each R₂₀ is independently H, or C₁-C₃ branched or unbranched alkyl;
R₁₄ is a heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H, C₁-C₃ alkyl, C₃-C₇ cycloalkyl, C₃-C₇ cycloalkenyl, optionally substituted with one or more NH and/or oxo groups, or R₁₀ and R₁₁ are taken together to form a heterocyclic ring;
R₁₆ is H, ═O, ═S, or CN;
each of s, u, and t is independently 1, 2, 3, 4, or 5;
each v is independently 0, 1, 2, 3, 4, or 5;
each Y is a divalent heterocyclic;
each Z is independently absent, O, S, or NR₁₂, wherein R₁₂ is H, C₁-C₇ branched or unbranched alkyl, or C₂-C₇ branched or unbranched alkenyl;
Q is O, S, CH₂, or NR₁₃, wherein each R₁₃ is H, or C₁-C₅ alkyl;
V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups; and
T is —NHC(O)O—, —OC(O)NH—, or a divalent heterocyclic; and 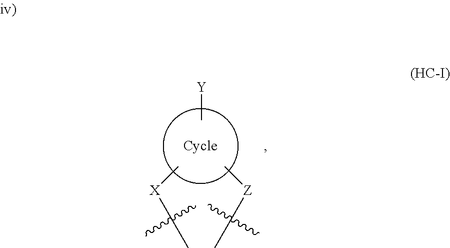
wherein: 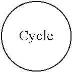
is cyclic or heterocyclic moiety;
Y is alkyl, hydroxy, hydroxyalkyl, 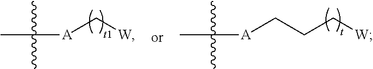
A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, or —S—S—;
each of X and Z is independently absent, —O—, —C(O)—, —N(R⁷)—, —O— alkylene-, -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, alkoxy, hydroxyalkyl, alkylamino, alkylaminoalkyl, or aminoalkyl;
t is 0, 1, 2, or 3;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl; and
wherein the lipid has a pKa from about 4 to about 8.

14. The nucleic acid vaccine of claim 13, wherein the ionizable lipid is a compound of group iv), and wherein at least one tail group of the lipid has one of the following formulas: 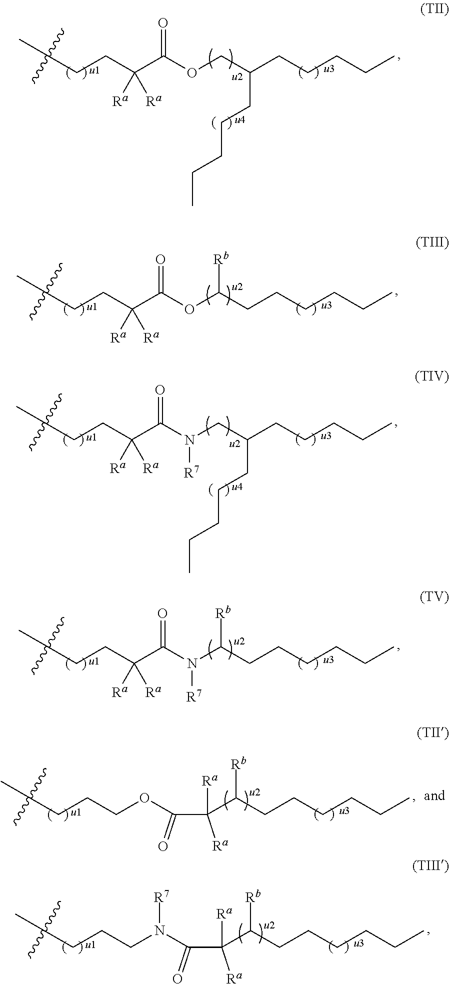
wherein:
R⁷ is each independently H or methyl;
R^{b} is in each occasion independently H or C₁-C₄ alkyl;
u1 and u2 are each independently 0, 1, 2, 3, 4, 5, 6, or 7; and
u3 and u4 are each independently 0, 1, 2, 3, 4, 5, 6, or 7; and
the head group has a structure of one of the following formulas: 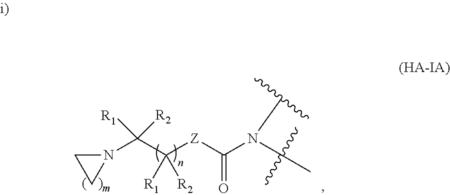
wherein m is 1, 2, 3, 4, 5, 6, 7 or 8;

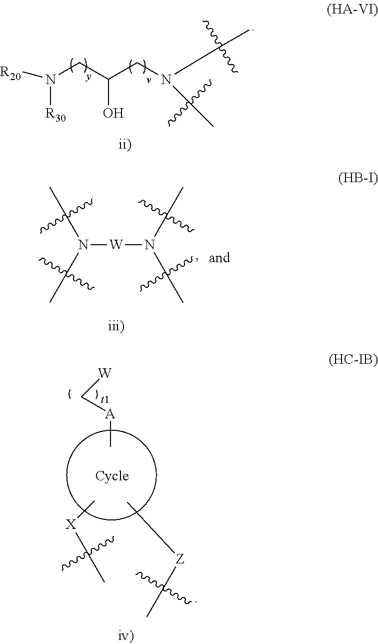

15-16. (canceled)

17. The nucleic acid vaccine of claim 2, wherein the ionizable lipid is

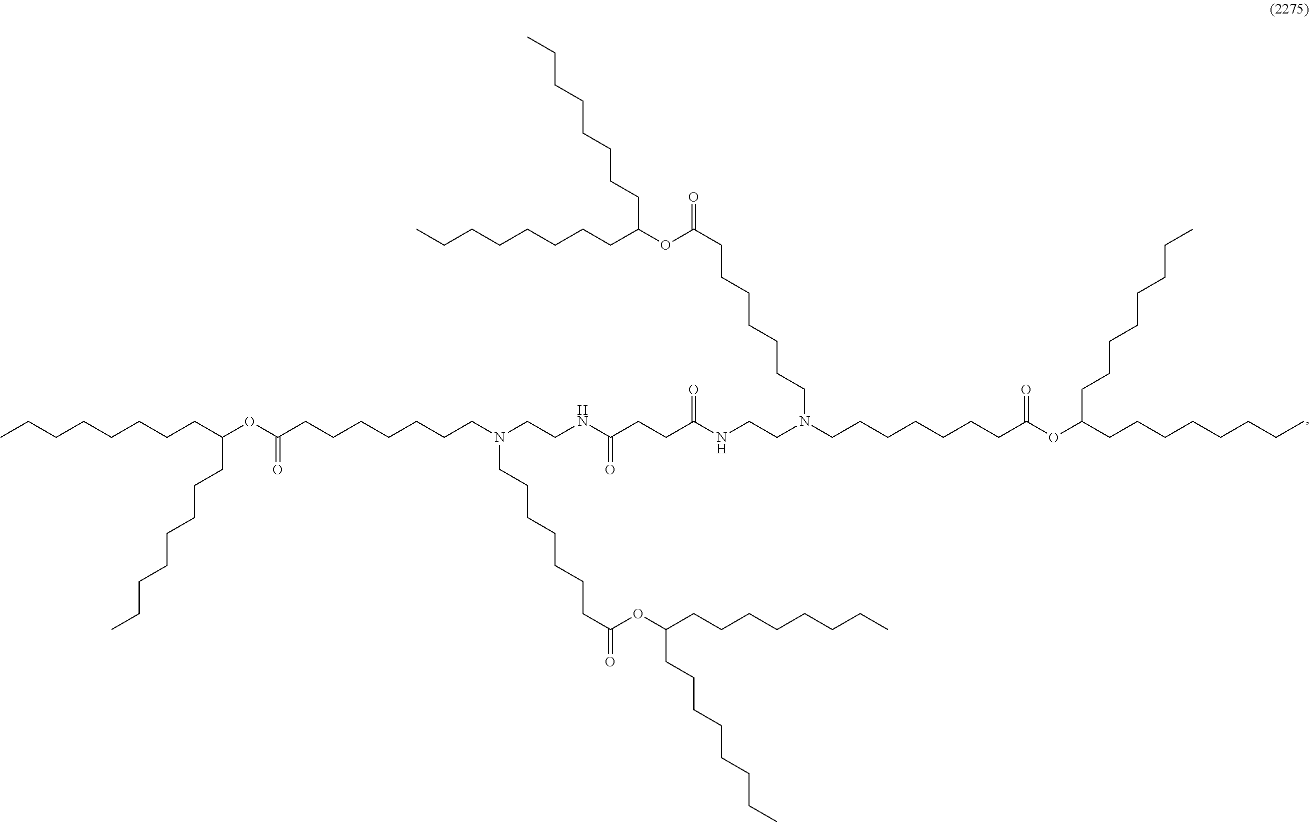 [SMILES: CCCCCCCCC(CCCCCCCC)OC(=O)CCCCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCCCC(=O)OC(CCCCCCCC)CCCCCCCC]

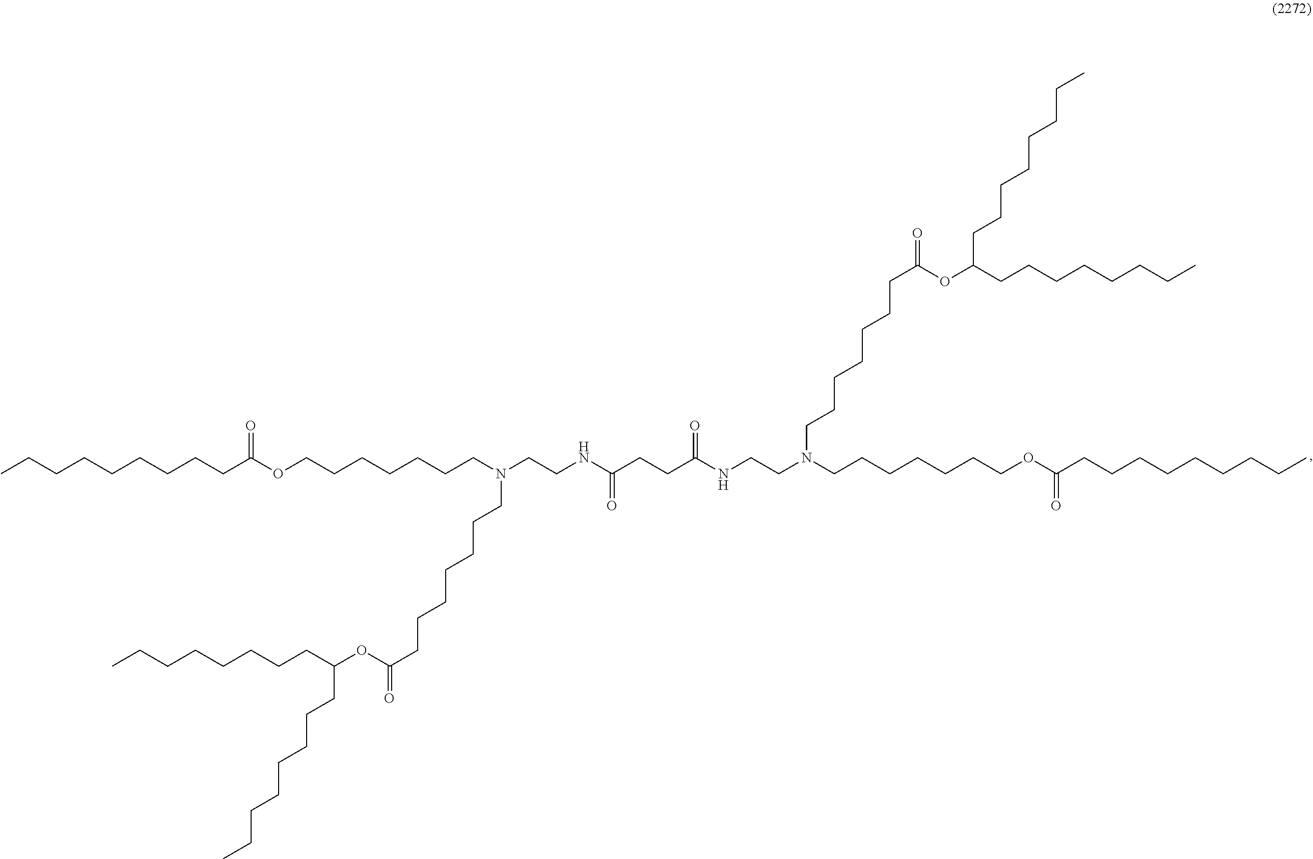 [SMILES: CCCCCCCCCC(=O)OCCCCCCCN(CCCCCCCC(=O)OC(CCCCCCCC)CCCCCCCC)CCNC(=O)CCC(=O)NCCN(CCCCCCCOC(=O)CCCCCCCCC)CCCCCCCC(=O)OC(CCCCCCCC)CCCCCCCC]

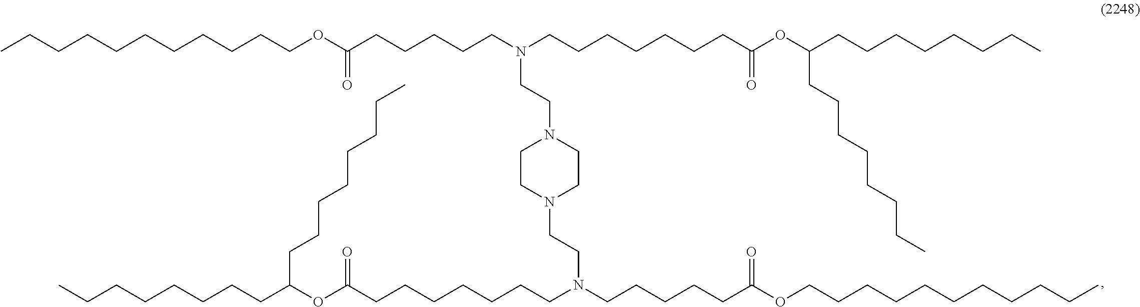 [SMILES: CCCCCCCCCCCOC(=O)CCCCCN(CCCCCCCC(=O)OC(CCCCCCCC)CCCCCCCC)CCN1CCN(CCN(CCCCCCCC(=O)OC(CCCCCCCC)CCCCCCCC)CCCCCC(=O)OCCCCCCCCCCC)CC1]

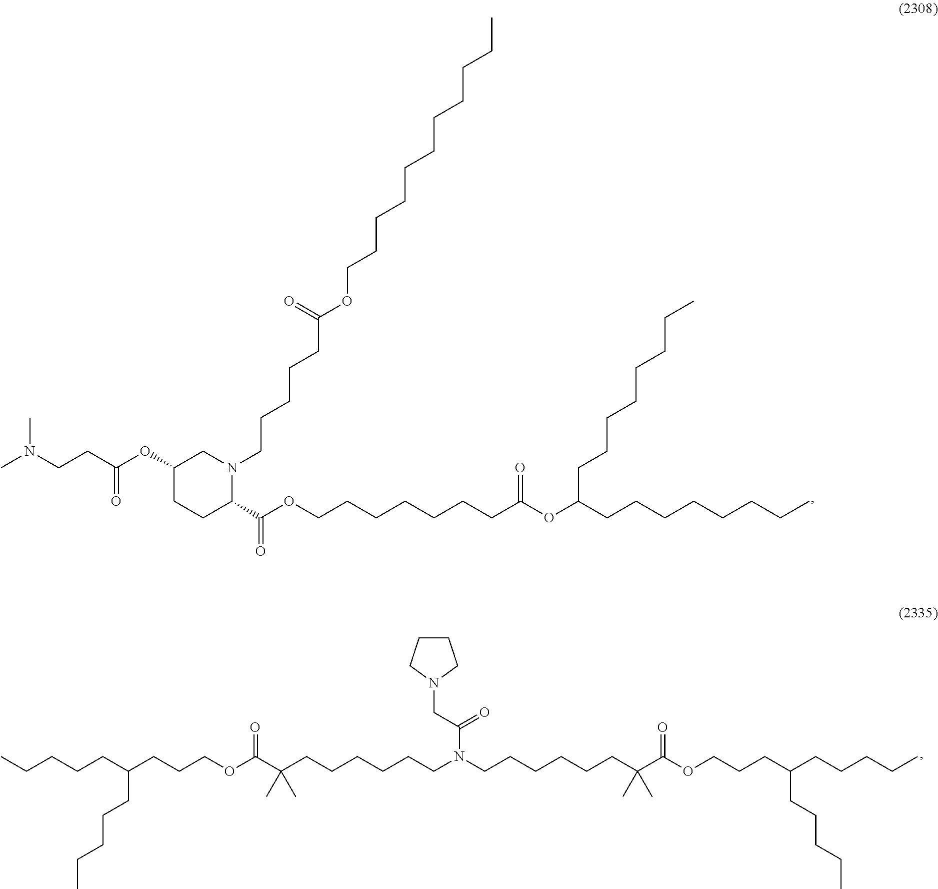

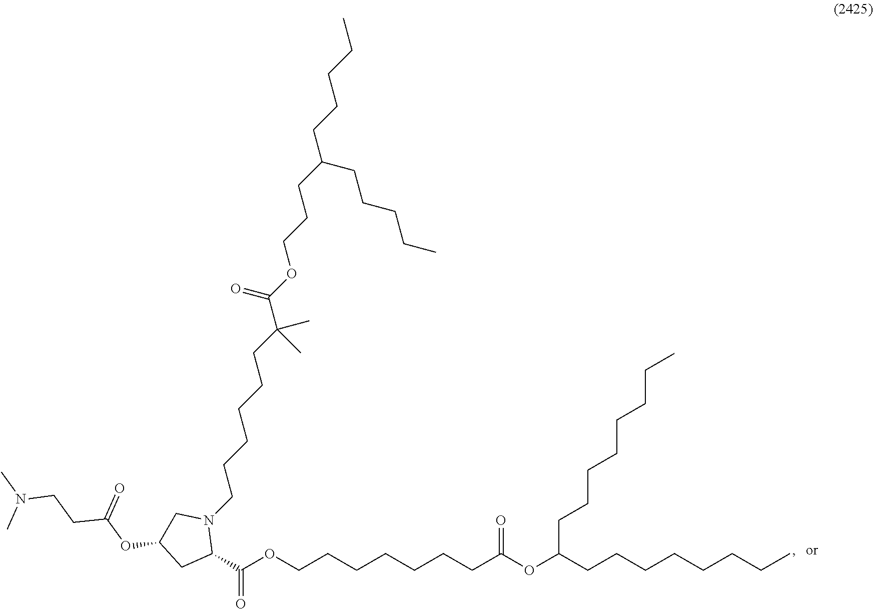 [SMILES: CCCCCCCCC(CCCCCCCC)OC(=O)CCCCCCCOC(=O)[C@@H]1C[C@H](OC(=O)CCN(C)C)CN1CCCCCCC(C)(C)C(=O)OCCCC(CCCCC)CCCCC]

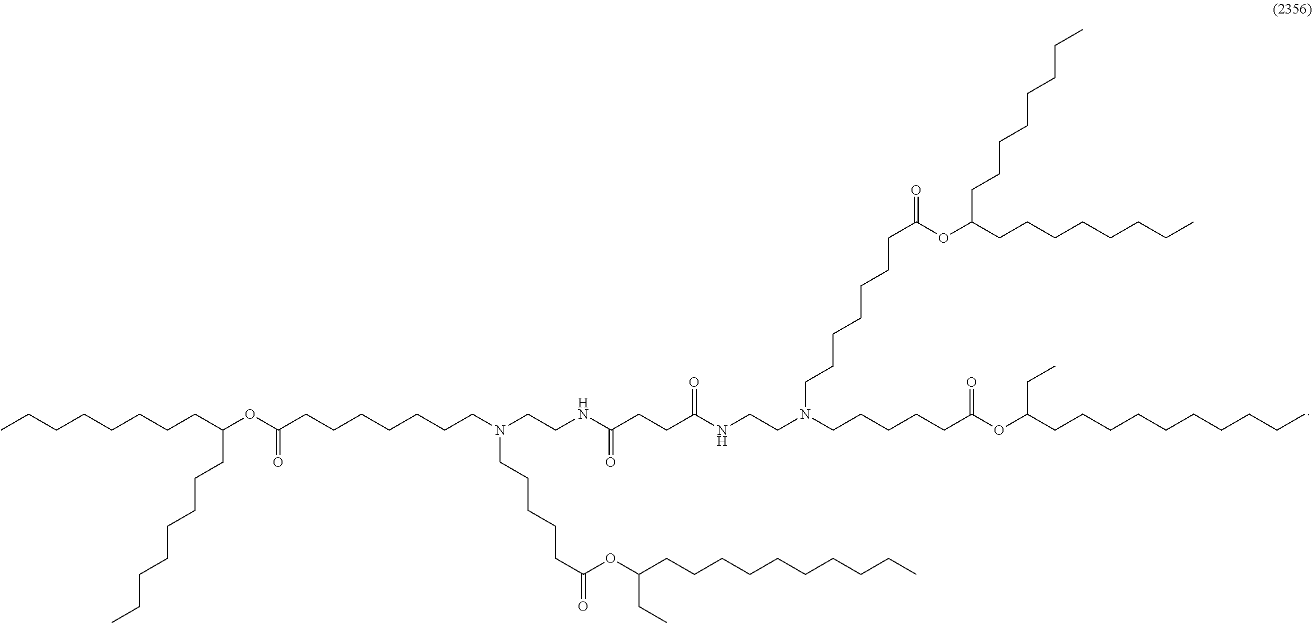 [SMILES: CCCCCCCCCCC(CC)OC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCC(=O)OC(CC)CCCCCCCCCC]

18-21. (canceled)

22. The nucleic acid vaccine of claim 2, wherein the LNMPs further comprise a sterol and a polyethylene glycol (PEG)-lipid conjugate, and wherein the LNMP comprises:
about 20 mol % to about 50 mol % of the ionizable lipid,
about 5 mol % to about 60 mol % of the natural lipids,
about 7 mol % to about 50 mol % of the sterol, and
about 0.5 mol % to about 3 mol % of the polyethylene glycol (PEG)-lipid conjugate.

23-25. (canceled)

26. The nucleic acid vaccine of claim 22, wherein the LNMPs comprise ionizable lipid:natural lipids:sterol:PEG-lipid at a molar ratio of about 35:50:12.5:2.5, about 35:20:42.5:2.5, about 50:20:28.5:1.5, about 45:20:33.5:2.5, or about 45:10:43.5:1.5.

27. The nucleic acid vaccine of claim 22, wherein the LNMPs further comprise a neutral lipid, and wherein the neutral lipid is a phospholipid selected from the group consisting of lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC, dioleoylphosphatidylcholine (DOPC, dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof.

28. (canceled)

29. The nucleic acid vaccine of claim 27, wherein the LNMP comprises:
about 20 mol % to about 50 mol % of the ionizable lipid,
about 5 mol % to about 60 mol % of the natural lipids and the neutral lipid,
about 7 mol % to about 50 mol % of the sterol, and
about 0.5 mol % to about 3 mol % of the polyethylene glycol (PEG)-lipid conjugate.

30. The nucleic acid vaccine of claim 29, wherein the LNMPs comprise ionizable lipid:(natural lipids+ neutral lipid):sterol:PEG-lipid at a molar ratio of about 35:50:12.5:2.5, of about 35:20:42.5:2.5, or of about 50:20:28.5:1.5.

31-53. (canceled)

54. The nucleic acid vaccine of claim 2, wherein the nucleic acid vaccine has at least 10%, lower liver expression than a lipid nanoparticle without natural lipids.

55. The nucleic acid vaccine of claim 2, wherein the nucleic acid vaccine induces a number of antigen-specific T cells in spleen cells at least higher than a lipid nanoparticle without natural lipids.

56. The nucleic acid vaccine of claim 2, wherein the nucleic acid vaccine reduces detectable infectious particles in the nares or in the lungs, wherein the detectable infectious particles are reduced 2-fold, or more compared to an unvaccinated control.

57-58. (canceled)

59. A method of preventing or reducing the transmission of an infectious disease, disorder, or condition, comprising:
administering to a subject the nucleic acid vaccine of claim 2, thereby preventing or reducing the transmission of an infectious disease, disorder, or condition.

60-61. (canceled)


SEQUENCE LISTING (35 sequences)

SEQ ID NO: 1 - DNA, 741 bp, synthetic construct
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaac

SEQ ID NO: 2 - DNA, 749 bp, synthetic construct
  source 1..749: mol_type = other DNA
ttaaaacagctgtgggttgtcacccacccacagggtccactgggcgctagtacactggtatctcggtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcggccacc

SEQ ID NO: 3 - protein, 8 aa, synthetic construct
  VARIANT 2: note = V or I
  VARIANT 4: note = Any amino acid
DXEXNPGP

SEQ ID NO: 4 - protein, 6 aa, synthetic construct
CHYSEL

SEQ ID NO: 5 - protein, 9 aa, synthetic construct
  VARIANT 1: note = G, H, or absent
  VARIANT 2: note = D, G, or absent
  VARIANT 3: note = D, V, I, S, or M
  VARIANT 5: note = Any amino acid
XXXEXNPGP

SEQ ID NO: 6 - protein, 8 aa, synthetic construct
  VARIANT 2: note = V or I
  VARIANT 4: note = Any amino acid
DXEXNPGP

SEQ ID NO: 7 - protein, 9 aa, synthetic construct
GDVESNPGP

SEQ ID NO: 8 - protein, 9 aa, synthetic construct
GDIEENPGP

SEQ ID NO: 9 - protein, 7 aa, synthetic construct
VEPNPGP

SEQ ID NO: 10 - protein, 7 aa, synthetic construct
IETNPGP

SEQ ID NO: 11 - protein, 9 aa, synthetic construct
GDIESNPGP

SEQ ID NO: 12 - protein, 9 aa, synthetic construct
GDVELNPGP

SEQ ID NO: 13 - protein, 9 aa, synthetic construct
GDIETNPGP

SEQ ID NO: 14 - protein, 8 aa, synthetic construct
GDVENPGP

SEQ ID NO: 15 - protein, 9 aa, synthetic construct
GDVEENPGP

SEQ ID NO: 16 - protein, 9 aa, synthetic construct
GDVEQNPGP

SEQ ID NO: 17 - protein, 7 aa, synthetic construct
IESNPGP

SEQ ID NO: 18 - protein, 9 aa, synthetic construct
GDIELNPGP

SEQ ID NO: 19 - protein, 9 aa, synthetic construct
HDIETNPGP

SEQ ID NO: 20 - protein, 9 aa, synthetic construct
HDVETNPGP

SEQ ID NO: 21 - protein, 9 aa, synthetic construct
HDVEMNPGP

SEQ ID NO: 22 - protein, 9 aa, synthetic construct
GDMESNPGP

SEQ ID NO: 23 - protein, 9 aa, synthetic construct
GDVETNPGP

SEQ ID NO: 24 - protein, 9 aa, synthetic construct
GDIEQNPGP

SEQ ID NO: 25 - protein, 8 aa, synthetic construct
DSEFNPGP

SEQ ID NO: 26 - DNA, 4885 bp, synthetic construct
  source 1..4885: mol_type = other DNA
aaaatccgtagcgtctaaacggtcgtgtgggttcaagtccctccacccccaaaaaaaataaaaaaaaattaaaaaaaaaatttaaaaaaaaattaaaaaaatttaaaaaaaattaaaaaaaaaataaaaaattaaaaaaataaaaaaaaaatttaaaaaaaaataaaaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaacatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggatttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatctttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctatcaacctcgtcagggacctgccgcagggcttcagcgctctggagcctctggtggatctccccatcgggatcaacatcacgaggttccagacgctgctggccctgcacaggtcctacctgaccccgggcgacagcagctcggggtggaccgcgggggcggcggcctactacgtgggctacctgcagcccaggacgttcttgttgaagtacaacgagaacggcacaatcaccgacgctgtggactgcgctctggatcctctctccgagactaagtgcaccctgaagtctttcaccgtggagaaggggatctaccagacctccaacttcagagtgcagcccacagagtcgattgtgcgcttccccaatatcacaaacctgtgcccctttggggaggtgttcaacgccacgcggttcgcgtccgtgtacgcctggaatcgcaagcgcatttccaactgcgtcgcggactactcggtgctgtacaactccgcctcgttctccaccttcaagtgctacggagtgtctccgaccaagctgaacgatctgtgtttcaccaacgtgtacgcagactcgttcgtgatcaggggggacgaggtgaggcagatagcaccgggccagacggggaagatcgctgattacaactacaagctccccgacgatttcaccggctgtgtgatcgcttggaactccaacaacctggattccaaggtcggggggaactacaactatctctatcgactcttcagaaagagcaatctgaagcccttcgagagagatatcagcactgagatctatcaggctgggagcaccccgtgcaacggggtggaggggttcaactgctacttcccactgcaatcctacggcttccagcccacgaacggcgtgggctatcagccttatagggttgtggtgctgagctttgagctcctccacgccccagccaccgtctgtggaccaaagaagtccacaaatctggtgaaaaataagtgcgtgaactttaatttcaacggactgacggggactggggtgctgacggagagcaacaagaagttcctgcccttccagcagttcggtcgtgacatcgccgacacgaccgacgctgtgagggacccccagacgctggagatcctggacatcactccctgcagctttgggggtgtctccgtcatcacgcctggtaccaacacttccaaccaggtggcagtgttgtaccaggacgtgaactgcacggaggtgcccgtggcaatccacgccgaccagttgaccccaacctggcgcgtgtattccacaggtagcaacgtgtttcagacccgggccggctgtctgattggcgccgagcacgtgaacaatagctacgagtgcgacatccccatcggggccggaatctgtgcgtcttaccagacgcagaccaattccccccggcgggctcgcagcgtagctagccagtcaatcatagcctacacgatgtccctgggcgccgagaactcggtggcctatagcaataactctattgctatccctactaacttcacgatctcggtcaccaccgagatcctgccagttagtatgaccaagacctccgtggattgcaccatgtacatctgtggtgattccacggagtgcagcaacctgctgctgcagtacggttcattctgcacccagctgaaccgtgccctgacaggcatcgccgtggagcaggacaagaacacccaggaggtgtttgcccaggtcaagcagatctacaagacacctcctatcaaggacttcggcggattcaacttctcccagatcctcccggatccgtcgaagccttccaagcggtccttcatcgaggacctcctgttcaacaaggtgaccctggccgacgccggcttcatcaagcagtacggcgactgcctgggggacatcgcggcccgggatctgatttgtgcccagaagttcaacgggctgactgtgcttccgcctctgctgaccgacgagatgatcgctcagtacacctccgccctgctggctgggaccatcaccagcgggtggacgttcggggctggagccgcgctccagatcccattcgcgatgcagatggcataccgcttcaacggaattggagtgacacagaacgtgctgtacgagaaccagaagctgatcgcgaaccagttcaactccgcgatcggcaagattcaggactccctgagctctaccgccagcgccctgggcaagctgcaggacgtggtcaaccagaacgcccaggcgctgaacaccctggtgaagcagctgtcctccaatttcggtgccatctcatccgtgctgaacgacatcttgtctcggctggaccctcccgaggcggaggtgcagatcgaccgcctcatcacggggcggctgcagtccctgcagacctacgtgactcagcagctcatcagggctgctgagatcagggccagcgccaatctggcagctaccaagatgtcagagtgcgtgctgggccagagcaagagggtggacttctgtggcaagggatatcacctgatgtcctttccacagagtgcaccccatggtgtggtgttcttgcacgtgacctacgtgcccgcccaggagaagaacttcacaaccgccccagccatctgccacgacgggaaggcccacttccctcgggagggggtcttcgtcagtaacggtactcactggttcgtgacccagaggaatttctacgagccccagatcatcaccactgacaatacctttgtgagtgggaactgcgacgtcgtgatcggtattgtgaacaataccgtgtacgacccgctgcagcctgagctggattctttcaaggaggagctggacaagtacttcaagaatcacacgtcccccgacgtggatctgggggatatctctggaatcaacgcctctgtggtcaacatccagaaggagatcgacaggctcaacgaggtggccaagaacctcaacgagagcctgatcgatctccaggagctcgggaagtacgagcagtacatcaagtggccgtggtacatctggctggggttcattgccggactcatcgccatcgtgatggtcacgatcatgctgtgctgcatgacttcctgctgcagctgcctcaagggctgctgctcttgcggcagctgctgcaagttcgacgaggacgactccgagcccgtcctcaagggagtcaagctgcactacacctaaataaaaaaaaattaaaaaaaaaatttaaaaaaaaattaaaaaaatttaaaaaaaattaaaaaaaaaataaaaaattaaaaaaataaaaaaaaaatttaaaaaaaaataaaaaagcaacccacacgaccgtttagacgctacggactt

SEQ ID NO: 27 - DNA, 5137 bp, synthetic construct
  source 1..5137: mol_type = other DNA
gggaacaacagataacttacagctagtcggaaggtgcagagactcgacgggagctaccctaacgtcaagacgagggtaaagagagagtccaattctcaaagccaataggcagtagcgaaagctgcgggagaatgaaaatccgtagcgtctaaacggtcgtgtgggttcaagtccctccacccccaaaaaaaataaaaaaaaattaaaaaaaaaatttaaaaaaaaattaaaaaaatttaaaaaaaattaaaaaaaaaataaaaaattaaaaaaataaaaaaaaaatttaaaaaaaaataaaaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaacatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggatttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatctttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctatcaacctcgtcagggacctgccgcagggcttcagcgctctggagcctctggtggatctccccatcgggatcaacatcacgaggttccagacgctgctggccctgcacaggtcctacctgaccccgggcgacagcagctcggggtggaccgcgggggcggcggcctactacgtgggctacctgcagcccaggacgttcttgttgaagtacaacgagaacggcacaatcaccgacgctgtggactgcgctctggatcctctctccgagactaagtgcaccctgaagtctttcaccgtggagaaggggatctaccagacctccaacttcagagtgcagcccacagagtcgattgtgcgcttccccaatatcacaaacctgtgcccctttggggaggtgttcaacgccacgcggttcgcgtccgtgtacgcctggaatcgcaagcgcatttccaactgcgtcgcggactactcggtgctgtacaactccgcctcgttctccaccttcaagtgctacggagtgtctccgaccaagctgaacgatctgtgtttcaccaacgtgtacgcagactcgttcgtgatcaggggggacgaggtgaggcagatagcaccgggccagacggggaagatcgctgattacaactacaagctccccgacgatttcaccggctgtgtgatcgcttggaactccaacaacctggattccaaggtcggggggaactacaactatctctatcgactcttcagaaagagcaatctgaagcccttcgagagagatatcagcactgagatctatcaggctgggagcaccccgtgcaacggggtggaggggttcaactgctacttcccactgcaatcctacggcttccagcccacgaacggcgtgggctatcagccttatagggttgtggtgctgagctttgagctcctccacgccccagccaccgtctgtggaccaaagaagtccacaaatctggtgaaaaataagtgcgtgaactttaatttcaacggactgacggggactggggtgctgacggagagcaacaagaagttcctgcccttccagcagttcggtcgtgacatcgccgacacgaccgacgctgtgagggacccccagacgctggagatcctggacatcactccctgcagctttgggggtgtctccgtcatcacgcctggtaccaacacttccaaccaggtggcagtgttgtaccaggacgtgaactgcacggaggtgcccgtggcaatccacgccgaccagttgaccccaacctggcgcgtgtattccacaggtagcaacgtgtttcagacccgggccggctgtctgattggcgccgagcacgtgaacaatagctacgagtgcgacatccccatcggggccggaatctgtgcgtcttaccagacgcagaccaattccccccggcgggctcgcagcgtagctagccagtcaatcatagcctacacgatgtccctgggcgccgagaactcggtggcctatagcaataactctattgctatccctactaacttcacgatctcggtcaccaccgagatcctgccagttagtatgaccaagacctccgtggattgcaccatgtacatctgtggtgattccacggagtgcagcaacctgctgctgcagtacggttcattctgcacccagctgaaccgtgccctgacaggcatcgccgtggagcaggacaagaacacccaggaggtgtttgcccaggtcaagcagatctacaagacacctcctatcaaggacttcggcggattcaacttctcccagatcctcccggatccgtcgaagccttccaagcggtccttcatcgaggacctcctgttcaacaaggtgaccctggccgacgccggcttcatcaagcagtacggcgactgcctgggggacatcgcggcccgggatctgatttgtgcccagaagttcaacgggctgactgtgcttccgcctctgctgaccgacgagatgatcgctcagtacacctccgccctgctggctgggaccatcaccagcgggtggacgttcggggctggagccgcgctccagatcccattcgcgatgcagatggcataccgcttcaacggaattggagtgacacagaacgtgctgtacgagaaccagaagctgatcgcgaaccagttcaactccgcgatcggcaagattcaggactccctgagctctaccgccagcgccctgggcaagctgcaggacgtggtcaaccagaacgcccaggcgctgaacaccctggtgaagcagctgtcctccaatttcggtgccatctcatccgtgctgaacgacatcttgtctcggctggaccctcccgaggcggaggtgcagatcgaccgcctcatcacggggcggctgcagtccctgcagacctacgtgactcagcagctcatcagggctgctgagatcagggccagcgccaatctggcagctaccaagatgtcagagtgcgtgctgggccagagcaagagggtggacttctgtggcaagggatatcacctgatgtcctttccacagagtgcaccccatggtgtggtgttcttgcacgtgacctacgtgcccgcccaggagaagaacttcacaaccgccccagccatctgccacgacgggaaggcccacttccctcgggagggggtcttcgtcagtaacggtactcactggttcgtgacccagaggaatttctacgagccccagatcatcaccactgacaatacctttgtgagtgggaactgcgacgtcgtgatcggtattgtgaacaataccgtgtacgacccgctgcagcctgagctggattctttcaaggaggagctggacaagtacttcaagaatcacacgtcccccgacgtggatctgggggatatctctggaatcaacgcctctgtggtcaacatccagaaggagatcgacaggctcaacgaggtggccaagaacctcaacgagagcctgatcgatctccaggagctcgggaagtacgagcagtacatcaagtggccgtggtacatctggctggggttcattgccggactcatcgccatcgtgatggtcacgatcatgctgtgctgcatgacttcctgctgcagctgcctcaagggctgctgctcttgcggcagctgctgcaagttcgacgaggacgactccgagcccgtcctcaagggagtcaagctgcactacacctaaataaaaaaaaattaaaaaaaaaatttaaaaaaaaattaaaaaaatttaaaaaaaattaaaaaaaaaataaaaaattaaaaaaataaaaaaaaaatttaaaaaaaaataaaaaagcaacccacacgaccgtttagacgctacggacttaaataattgagccttagagaagaaattctttaagtggatgctctcaaactcagggaaacctaaatctagctatagacaaggcaatcctgagccaagccgaagtagtaattagtaagtt

SEQ ID NO: 28 - RNA, 4308 nt, synthetic construct
  source 1..4308: mol_type = other RNA
taatacgactcactataaggagaataaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaacggct

SEQ ID NO: 29 - RNA, 582 nt, synthetic construct
  source 1..582: mol_type = other RNA
atgggcgtgcacgagtgccccgcctggctgtggctgctgctgagcctgctgagcctgcccctgggcctgcccgtgctgggcgcccccccccggctgatctgcgacagccgggtgctggagcggtacctgctggaggccaaggaggccgagaacatcaccaccggctgcgccgagcactgcagcctgaacgagaacatcaccgtgcccgacaccaaggtgaacttctacgcctggaagcggatggaggtgggccagcaggccgtggaggtgtggcagggcctggccctgctgagcgaggccgtgctgcggggccaggccctgctggtgaacagcagccagccctgggagcccctgcagctgcacgtggacaaggccgtgagcggcctgcggagcctgaccaccctgctgcgggccctgggcgcccagaaggaggccatcagcccccccgacgccgccagcgccgcccccctgcggaccatcaccgccgacaccttccggaagctgttccgggtgtacagcaacttcctgcggggcaagctgaagctgtacaccggcgaggcctgccggaccggcgaccggtga

SEQ ID NO: 30 - RNA, 1653 nt, synthetic construct
  source 1..1653: mol_type = other RNA
atggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtga

SEQ ID NO: 31 - RNA, 11 nt, synthetic construct
  source 1..11: mol_type = other RNA
gggatcctacc

SEQ ID NO: 32 - RNA, 30 nt, synthetic construct
  source 1..30: mol_type = other RNA
aaaaaaaaaaaaaaaaaaaaaaaaaaaaaa

SEQ ID NO: 33 - RNA, 70 nt, synthetic construct
  source 1..70: mol_type = other RNA
aaaaaaaaaaaaaaaaaaaaaaaaaaaaaaaaaaaaaaaaaaaaaaaaaaaaaaaaaaaaaaaaaaaaaa

SEQ ID NO: 34 - RNA, 200 nt, synthetic construct
  source 1..200: mol_type = other RNA
  misc_difference 1..200: note = This sequence may encompass 100-200 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35 - RNA, 250 nt, synthetic construct
  source 1..250: mol_type = other RNA
  misc_difference 1..250: note = This sequence may encompass 50-250 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa